SEQUENCE LISTING (320 sequences)

SEQ ID NO: 1 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 2 - protein, 778 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAEPVEDNVINFVAMKFIDNTLYFIAEDDENDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 3 - protein, 778 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAEPVEDNVINFVAMKFIDNTLYFIAEDDENDDDDKYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 4 - protein, 748 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 5 - protein, 748 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGDDDDKYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 6 - protein, 425 aa, synthetic construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 7 - protein, 394 aa, synthetic construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 8 - protein, 422 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAAEPVEDNVINFVAMKFIDNTLYFIAEDDENDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 9 - protein, 391 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 10 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 11 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 12 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 13 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKAAEPVEDNVINFVAMKFIDNTLYFIAEDDENIGNRSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 14 - protein, 439 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKAAEPVEDNVINFVAMKFIDNTLYFIAEDDENGGGGKPLGLQARVVGGGGTYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 15 - protein, 455 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKAAEPVEDNVINFVAMKFIDNTLYFIAEDDENIEQDGGSIGNRSGGGGKPLGLQARVVGGGGTIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 16 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 664 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGGSGGGGSGGGGSGGGGSGGGGSAAEPVEDNSINFVAMKFIDNTLYFIAEDDENIEQDYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 18 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMSSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 19 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 20 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNSINFVAMKFIDNTLYFIAEDDENDDDDKYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 21 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNAINFVAMKFIDNTLYFIAEDDENDDDDKYFGKLESKLSVIRNLNDQVLFIDQGNRPLFEDMTDSDARDNAPRTIFIISKYGDSQPRGLAVTISVKAEKISTLSAENKIISFKEMNPPDNIKDTKSDIIFFQRSVPGHDNKVQFESSSYEGYFLAAEKERDLFKLILKKEDELGDRSIMFTVQNED

SEQ ID NO: 22 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENDDDDKYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 23 - protein, 395 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGDDDDKYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 24 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 25 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 26 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNSINFVAMKFIDNTLYFIAEDDENDDDDKYFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 27 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNAINFVAMKFIDNTLYFIAEDDENDDDDKYFGKLESKLSVIRNLNDQVLFIDQGNRPLFEDMTDSDARDNAPRTIFIISMYKDSQPRGMAVTISVKAEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 425 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENDDDDKYFGKLESKLSVIRNLNDQVLFIDQGNRPLFEDMTDSDVRDNAPRTIFIISKYGDSGARGLAVTISVKVEKISTLSVENKIISFKEMNPPDNIKDTKSDIIFFERDVPGHSGKVQFESSSYEGYFLAVEKERDLFKLILKKEDELGDRSIMFTVQNED

SEQ ID NO: 29 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 30 - protein, 428 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENGPLGVRGGYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 31 - protein, 432 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENVHMPLGFLGPGGYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 32 - protein, 424 aa, synthetic construct
AAEPVEDNVINFVAMKFIDNTLYFIAEDDENLESDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 389 aa, synthetic construct
YFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 394 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKDDDDKYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 35 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 36 - protein, 641 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 37 - protein, 389 aa, synthetic construct
YFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 389 aa, synthetic construct
YFGKLKSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 389 aa, synthetic construct
YFGKLRSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 661 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 41 - protein, 359 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 389 aa, synthetic construct
YFGKLASKLSVIRNLNDQVLFIDQGNRPLFEDMTDSDVRDNAPRTIFIISMYADSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 389 aa, synthetic construct
YFGKLHSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 389 aa, synthetic construct
YFGKLRSKLSVIRNLNDQVLFIDQGNRPLFEDMTDSDVRDNAPRTIFIISMYEDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 389 aa, synthetic construct
YFGKLQSKLSVIRNLNDQVLFIDQGNRPLFEDMTDSDVRDNAPRTIFIISMYADSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 443 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENGGGGKPLGLQARVVGGGGTIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 47 - protein, 389 aa, synthetic construct
YFGKLKSKLSVIRNLNDQVLFIDQGNRPLFEDMTDSDVRDNAPRTIFIISHYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 389 aa, synthetic construct; identical to SEQ ID NO: 47

SEQ ID NO: 49 - protein, 389 aa, synthetic construct
YFGKLESKLSVIRNLNDQVLFIDQGNRPLFEDMTDSDVRDNAPRTIFIISHYADSQPRGIAVTISVKVEKISTLSVENKIISFKEMNPPDNIKDTKSDIIFFQRSVPGHDNKIQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 389 aa, synthetic construct
YFGKLESKLSVIRNLNDQVLFIDQGNRPLFEDMTDSDVRDNAPRTIFIISKYADSQPRGIAVTISVKVEKISTLSVENKIISFKEMNPPDNIKDTKSDIIFFQRSVPGHDNKIQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 680 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAAEPVEDNVINFVAMKFIDNTLYFIAEDDENGGGGKPLGLQARVVGGGGTIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 52 - protein, 681 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAAEPVEDNVINFVAMKFIDNTLYFIAEDDENGGGGKPLGLQARVVGGGGTIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 53 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 54 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNSINFVAMKFIDNTLYFIAEDDENIEQDYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 55 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNSINFVAMKFIDNTLYFIAENDENQDIEYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 56 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 424 aa, synthetic construct
GGGGSGGGGSGGGGSGGGGSGGGGSGGGGSGIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 404 aa, synthetic construct
GGGGSGGGGSGIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 404 aa, synthetic construct
LQARVVGGGGTIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMSSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAENDENGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 61 - protein, 654 aa, synthetic construct
QVQLVESGGGVVQPGRSLRLDCKASGITFSNSGMHWVRQAPGKGLEWVAVIWYDGSKRYYADSVKGRFTISRDNSKNTLFLQMSSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAAEPVEDNSINFVAMKFIDNTLYFIAENDENGGGSYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 62 - protein, 654 aa, synthetic construct
QVQLVESGGGVVQPGRSLRLDCKASGITFSNSGMHWVRQAPGKGLEWVAVIWYDGSKRYYADSVKGRFTISRDNSKNTLFLQMSSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAAEPVEDNVINFVAMKFIDNTLYFIAENDENGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 63 - protein, 428 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGSGGGGAAEPVEDNSINFVAMKFIDNTLYFIAENDENGGGSYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 64 - protein, 390 aa, synthetic construct
SYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 394 aa, synthetic construct
GGGGSYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 399 aa, synthetic construct
GGGGSGGGGSYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNTINFVAMKFIDNTLYFIAEDDENLESDYFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 68 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNQINFVAMKFIDNTLYFIAEDDENLESDYFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 69 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNDINFVAMKFIDNTLYFIAEDDENLESDYFGKLESKLSVIRNLNDQVLFIDQGNRPLFEDMTDSDARDNAPRTIFIISMYKDSQPRGMAVTISVKAEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 70 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNFINFVAMKFIDNTLYFIAEDDENLESDYFGKLESKLSVIRNLNDQVLFIDQGNRPLFEDMTDSDARDNAPRTIFIISMYKDSQPRGMAVTISVKAEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 71 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNLINFVAMKFIDNTLYFIAEDDENLESDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 72 - protein, 424 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNHINFVAMKFIDNTLYFIAEDDENLESDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 73 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 165 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTE

SEQ ID NO: 75 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 31 aa, synthetic construct
AAEPVEDNSINFVAMKFIDNTLYFIAEDDEN

SEQ ID NO: 77 - protein, 31 aa, synthetic construct
AAEPVEDNAINFVAMKFIDNTLYFIAEDDEN

SEQ ID NO: 78 - protein, 31 aa, synthetic construct
AAEPVEDNVINFVAMKFIDNTLYFIAEDDEN

SEQ ID NO: 79 - protein, 31 aa, synthetic construct
AAEPVEDNSINFVAMKFIDNTLYFIAENDEN

SEQ ID NO: 80 - protein, 31 aa, synthetic construct
AAEPVEDNVINFVAMKFIDNTLYFIAENDEN

SEQ ID NO: 81 - protein, 35 aa, synthetic construct
AAEPVEDNTINFVAMKFIDNTLYFIAEDDENLESD

SEQ ID NO: 82 - protein, 35 aa, synthetic construct
AAEPVEDNQINFVAMKFIDNTLYFIAEDDENLESD

SEQ ID NO: 83 - protein, 35 aa, synthetic construct
AAEPVEDNDINFVAMKFIDNTLYFIAEDDENLESD

SEQ ID NO: 84 - protein, 35 aa, synthetic construct
AAEPVEDNFINFVAMKFIDNTLYFIAEDDENLESD

SEQ ID NO: 85 - protein, 35 aa, synthetic construct
AAEPVEDNLINFVAMKFIDNTLYFIAEDDENLESD

SEQ ID NO: 86 - protein, 35 aa, synthetic construct
AAEPVEDNHINFVAMKFIDNTLYFIAEDDENLESD

SEQ ID NO: 87 - protein, 5 aa, synthetic construct
DDDDK

SEQ ID NO: 88 - protein, 4 aa, synthetic construct
IEQD

SEQ ID NO: 89 - protein, 6 aa, synthetic construct
GPLGVR

SEQ ID NO: 90 - protein, 10 aa, synthetic construct
VHMPLGFLGP

SEQ ID NO: 91 - protein, 4 aa, synthetic construct
LESD

SEQ ID NO: 92 - protein, 19 aa, synthetic construct
KPLGLQARVVGGGGTIEQD

SEQ ID NO: 93 - protein, 5 aa, synthetic construct
IGNRS

SEQ ID NO: 94 - protein, 10 aa, synthetic construct
KPLGLQARVV

SEQ ID NO: 95 - protein, 35 aa, synthetic construct
IEQDGGSIGNRSGGGGKPLGLQARVVGGGGTIEQD

SEQ ID NO: 96 - protein, 4 aa, synthetic construct
QDIE

SEQ ID NO: 97 - protein, 157 aa, synthetic construct
YFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 98 - protein, 157 aa, synthetic construct
YFGKLESKLSVIRNLNDQVLFIDQGNRPLFEDMTDSDARDNAPRTIFIISKYGDSQPRGLAVTISVKAEKISTLSAENKIISFKEMNPPDNIKDTKSDIIFFQRSVPGHDNKVQFESSSYEGYFLAAEKERDLFKLILKKEDELGDRSIMFTVQNED

SEQ ID NO: 99 - protein, 157 aa, synthetic construct
YFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 100 - protein, 157 aa, synthetic construct
YFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 101 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 102 - protein, 157 aa, synthetic construct
YFGKLESKLSVIRNLNDQVLFIDQGNRPLFEDMTDSDARDNAPRTIFIISMYKDSQPRGMAVTISVKAEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 103 - protein, 157 aa, synthetic construct
YFGKLESKLSVIRNLNDQVLFIDQGNRPLFEDMTDSDVRDNAPRTIFIISKYGDSGARGLAVTISVKVEKISTLSVENKIISFKEMNPPDNIKDTKSDIIFFERDVPGHSGKVQFESSSYEGYFLAVEKERDLFKLILKKEDELGDRSIMFTVQNED

SEQ ID NO: 104 - protein, 157 aa, synthetic construct
YFGKLKSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 105 - protein, 157 aa, synthetic construct
YFGKLRSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 106 - protein, 157 aa, synthetic construct
YFGKLASKLSVIRNLNDQVLFIDQGNRPLFEDMTDSDVRDNAPRTIFIISMYADSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 107 - protein, 157 aa, synthetic construct
YFGKLHSKLSVIRNLNDQVLFIDQGNRPLFEDMTDSDVRDNAPRTIFIISMYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 108 - protein, 157 aa, synthetic construct
YFGKLRSKLSVIRNLNDQVLFIDQGNRPLFEDMTDSDVRDNAPRTIFIISMYEDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 109 - protein, 157 aa, synthetic construct
YFGKLQSKLSVIRNLNDQVLFIDQGNRPLFEDMTDSDVRDNAPRTIFIISMYADSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 110 - protein, 157 aa, synthetic construct
YFGKLKSKLSVIRNLNDQVLFIDQGNRPLFEDMTDSDVRDNAPRTIFIISHYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 111 - protein, 157 aa, synthetic construct
YFGKLKSKLSVIRNLNDQVLFIDQGNRPLFEDMTDSDVRDNAPRTIFIISKYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 112 - protein, 157 aa, synthetic construct
YFGKLESKLSVIRNLNDQVLFIDQGNRPLFEDMTDSDVRDNAPRTIFIISHYADSQPRGIAVTISVKVEKISTLSVENKIISFKEMNPPDNIKDTKSDIIFFQRSVPGHDNKIQFESSSYEGYFLAVEKERDLFKLILKKEDELGDRSIMFTVQNED

SEQ ID NO: 113 - protein, 157 aa, synthetic construct
YFGKLESKLSVIRNLNDQVLFIDQGNRPLFEDMTDSDVRDNAPRTIFIISKYADSQPRGIAVTISVKVEKISTLSVENKIISFKEMNPPDNIKDTKSDIIFFQRSVPGHDNKIQFESSSYEGYFLAVEKERDLFKLILKKEDELGDRSIMFTVQNED

SEQ ID NO: 114 - DNA, 696 bp, synthetic construct
  source 1..696: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 115 - DNA, 2334 bp, synthetic construct
  source 1..2334: mol_type = other DNA
gacgctcacaaatccgaagtggcacacaggtttaaggacctgggagaagagaacttcaaagccctggtgctgattgcatttgcccagtatctgcagcagtgtccctttgaggaccacgtgaagctggtgaacgaggtgaccgaatttgccaagacttgcgtggccgacgagtccgcagagaactgcgacaagtccctgcataccctgtttggcgacaagctgtgtacagtggccaccctgcgggagacatacggtgagatggccgactgctgcgccaagcaggaacccgagagaaacgaatgcttcctgcagcacaaggacgacaaccccaacctgccccggctggtgaggcccgaagtggacgtgatgtgtaccgcctttcacgacaacgaagagacatttctgaagaagtacctgtacgagatcgccagaaggcacccttacttttacgcccccgagctgctgttctttgccaagaggtataaggccgcctttactgagtgctgccaggcagccgacaaagccgcctgcctgctgcccaagctggacgagctgcgcgacgagggcaaggcctcctccgctaagcagaggctgaagtgcgcatccctgcagaaatttggcgagagagccttcaaggcatgggccgtggctaggctgtcccagaggtttccaaaggccgaatttgccgaggtgtccaagctggtgacagacctgaccaaggtgcacaccgagtgttgtcacggagacttgctggagtgcgccgacgacagggccgacctggctaagtacatctgtgagaaccaggactctatctcctccaagctgaaggagtgctgcgagaagcccctgctggagaaaagccactgtattgctgaggtggagaacgacgagatgcccgccgacctgccctctctggccgctgatttcgtggagtccaaggacgtgtgtaagaactacgccgaggccaaagacgtgttcctggggatgtttctgtatgagtacgctaggaggcaccccgattactccgtggtgctgctgctgaggctggcaaaaacatacgaaaccaccctggagaagtgctgtgccgctgctgacccccacgagtgctatgccaaggtgttcgacgagttcaaacccctggtggaggaaccccagaacctgatcaagcagaactgcgagctgttcgaacagctgggcgagtacaaattccagaacgcactgctggtgaggtacaccaagaaagtgccccaggtgagcacccccaccctggtggaggtgtccaggaacctgggcaaagtgggcagcaaatgctgcaagcaccccgaggccaaaagaatgccctgtgcagaggactacctgagtgtggtgctgaaccagctgtgcgtgctgcacgagaagacacccgtgagcgacagggtgaccaagtgttgtaccgagtccctggtgaacagaaggccctgttttagtgccctggaagtggatgagacttacgtgcctaaggagttcaacgcagagacattcaccttccatgccgatatttgcaccctgtccgaaaaggaaagacagatcaagaaacagaccgccctggtggagctggtgaaacacaagcccaaggccaccaaggaacagctgaaagccgtgatggacgacttcgccgccttcgtggagaaatgctgcaaggccgacgacaaagaaacctgcttcgcagaggaaggaaagaaactggtggcagccagccaggccgccctgggactggctgctgaacctgtggaagataatgtgatcaatttcgtggccatgaagttcattgacaacaccctgtacttcatcgctgaagatgatgaaaacgacgatgatgataagtacttcggaaagctggagtccaagctgagcgtgattaggaacctgaatgaccaggtgctgttcattgaccagggcaacaggcccctctttgaggatatgaccgactccgatgtgagggacaacgcccctagaacaatcttcatcatctccatgtacaaggactcccagccaaggggaatggccgtgaccatttccgtgaaagtcgagaaaatttccaccctgtccgtggagaacaagatcatctccttcaaagaaatgaacccccccgacaacatcaaggacaccaaatccgacatcatcttcttccagcgctccgtgccaggccatgataacaaaatgcagttcgagagcagctcctacgagggctacttcctggcagtcgaaaaggaaagggacctgttcaagctgatcctgaagaaggaggacgagctgggggacagaagcatcatgttcaccgtgcagaacgaagac

SEQ ID NO: 116 - DNA, 2334 bp, synthetic construct
  source 1..2334: mol_type = other DNA
gacgctcacaaatccgaagtggcacacaggtttaaggacctgggagaagagaacttcaaagccctggtgctgattgcatttgcccagtatctgcagcagtgtccctttgaggaccacgtgaagctggtgaacgaggtgaccgaatttgccaagacttgcgtggccgacgagtccgcagagaactgcgacaagtccctgcataccctgtttggcgacaagctgtgtacagtggccaccctgcgggagacatacggtgagatggccgactgctgcgccaagcaggaacccgagagaaacgaatgcttcctgcagcacaaggacgacaaccccaacctgccccggctggtgaggcccgaagtggacgtgatgtgtaccgcctttcacgacaacgaagagacatttctgaagaagtacctgtacgagatcgccagaaggcacccttacttttacgcccccgagctgctgttctttgccaagaggtataaggccgcctttactgagtgctgccaggcagccgacaaagccgcctgcctgctgcccaagctggacgagctgcgcgacgagggcaaggcctcctccgctaagcagaggctgaagtgcgcatccctgcagaaatttggcgagagagccttcaaggcatgggccgtggctaggctgtcccagaggtttccaaaggccgaatttgccgaggtgtccaagctggtgacagacctgaccaaggtgcacaccgagtgttgtcacggagacttgctggagtgcgccgacgacagggccgacctggctaagtacatctgtgagaaccaggactctatctcctccaagctgaaggagtgctgcgagaagcccctgctggagaaaagccactgtattgctgaggtggagaacgacgagatgcccgccgacctgccctctctggccgctgatttcgtggagtccaaggacgtgtgtaagaactacgccgaggccaaagacgtgttcctggggatgtttctgtatgagtacgctaggaggcaccccgattactccgtggtgctgctgctgaggctggcaaaaacatacgaaaccaccctggagaagtgctgtgccgctgctgacccccacgagtgctatgccaaggtgttcgacgagttcaaacccctggtggaggaaccccagaacctgatcaagcagaactgcgagctgttcgaacagctgggcgagtacaaattccagaacgcactgctggtgaggtacaccaagaaagtgccccaggtgagcacccccaccctggtggaggtgtccaggaacctgggcaaagtgggcagcaaatgctgcaagcaccccgaggccaaaagaatgccctgtgcagaggactacctgagtgtggtgctgaaccagctgtgcgtgctgcacgagaagacacccgtgagcgacagggtgaccaagtgttgtaccgagtccctggtgaacagaaggccctgttttagtgccctggaagtggatgagacttacgtgcctaaggagttcaacgcagagacattcaccttccatgccgatatttgcaccctgtccgaaaaggaaagacagatcaagaaacagaccgccctggtggagctggtgaaacacaagcccaaggccaccaaggaacagctgaaagccgtgatggacgacttcgccgccttcgtggagaaatgctgcaaggccgacgacaaagaaacctgcttcgcagaggaaggaaagaaactggtggcagccagccaggccgccctgggactggctgctgaacctgtggaagataatgtgatcaatttcgtggccatgaagttcattgacaacaccctgtacttcatcgctgaagatgatgaaaacgacgatgatgataagtacttcggaaagctggagtccaagctgagcgtgattaggaacctgaatgaccaggtgctgttcattgaccagggcaacaggcccctctttgaggatatgaccgactccgatgtgagggacaacgcccctagaacaatcttcatcatctccaagtacggcgactcccagccaagaggactggctgtgacaattagcgtgaaagtcgagaagatttccaccctgtccgtggagaacaaaatcatctccttcaaggagatgaacccccccgacaacatcaaggacaccaaaagcgatatcatcttcttccagaggtccgtccccggtcacgataacaaagtgcagttcgagtcctcctcctacgagggttactttctggccgtcgaaaaggagcgggacctgtttaagctgatcctgaagaaggaggacgagctgggtgacaggtccatcatgttcaccgtccagaatgaggac

SEQ ID NO: 117 - DNA, 2244 bp, synthetic construct
  source 1..2244: mol_type = other DNA
gacgctcacaaatccgaagtggcacacaggtttaaggacctgggagaagagaacttcaaagccctggtgctgattgcatttgcccagtatctgcagcagtgtccctttgaggaccacgtgaagctggtgaacgaggtgaccgaatttgccaagacttgcgtggccgacgagtccgcagagaactgcgacaagtccctgcataccctgtttggcgacaagctgtgtacagtggccaccctgcgggagacatacggtgagatggccgactgctgcgccaagcaggaacccgagagaaacgaatgcttcctgcagcacaaggacgacaaccccaacctgccccggctggtgaggcccgaagtggacgtgatgtgtaccgcctttcacgacaacgaagagacatttctgaagaagtacctgtacgagatcgccagaaggcacccttacttttacgcccccgagctgctgttctttgccaagaggtataaggccgcctttactgagtgctgccaggcagccgacaaagccgcctgcctgctgcccaagctggacgagctgcgcgacgagggcaaggcctcctccgctaagcagaggctgaagtgcgcatccctgcagaaatttggcgagagagccttcaaggcatgggccgtggctaggctgtcccagaggtttccaaaggccgaatttgccgaggtgtccaagctggtgacagacctgaccaaggtgcacaccgagtgttgtcacggagacttgctggagtgcgccgacgacagggccgacctggctaagtacatctgtgagaaccaggactctatctcctccaagctgaaggagtgctgcgagaagcccctgctggagaaaagccactgtattgctgaggtggagaacgacgagatgcccgccgacctgccctctctggccgctgatttcgtggagtccaaggacgtgtgtaagaactacgccgaggccaaagacgtgttcctggggatgtttctgtatgagtacgctaggaggcaccccgattactccgtggtgctgctgctgaggctggcaaaaacatacgaaaccaccctggagaagtgctgtgccgctgctgacccccacgagtgctatgccaaggtgttcgacgagttcaaacccctggtggaggaaccccagaacctgatcaagcagaactgcgagctgttcgaacagctgggcgagtacaaattccagaacgcactgctggtgaggtacaccaagaaagtgccccaggtgagcacccccaccctggtggaggtgtccaggaacctgggcaaagtgggcagcaaatgctgcaagcaccccgaggccaaaagaatgccctgtgcagaggactacctgagtgtggtgctgaaccagctgtgcgtgctgcacgagaagacacccgtgagcgacagggtgaccaagtgttgtaccgagtccctggtgaacagaaggccctgttttagtgccctggaagtggatgagacttacgtgcctaaggagttcaacgcagagacattcaccttccatgccgatatttgcaccctgtccgaaaaggaaagacagatcaagaaacagaccgccctggtggagctggtgaaacacaagcccaaggccaccaaggaacagctgaaagccgtgatggacgacttcgccgccttcgtggagaaatgctgcaaggccgacgacaaagaaacctgcttcgcagaggaaggaaagaaactggtggcagccagccaggccgccctgggactgggagatgacgacgacaagtactttggaaagctggagagcaagctgagtgtgatcaggaacctgaacgaccaggtgctgtttatcgaccagggcaataggccactgtttgaggatatgacagattccgacgtgagggacaacgctcccagaacaattttcatcatctctatgtacaaggactcccagccaaggggcatggctgtgaccatttccgtgaaggtggagaagatttccaccctgtctgtggagaacaagatcatctccttcaaagagatgaacccccccgacaacatcaaagacaccaaaagcgatatcatcttcttccagaggtccgtgcccggtcacgataacaaaatgcagttcgaaagctcctcctacgagggatactttctggccgtcgaaaaggaacgcgacctgttcaagctgatcctgaagaaggaggacgagctgggggacagaagcatcatgttcaccgtgcagaacgaagac

SEQ ID NO: 118 - DNA, 2244 bp, synthetic construct
  source 1..2244: mol_type = other DNA
gacgctcacaaatccgaagtggcacacaggtttaaggacctgggagaagagaacttcaaagccctggtgctgattgcatttgcccagtatctgcagcagtgtccctttgaggaccacgtgaagctggtgaacgaggtgaccgaatttgccaagacttgcgtggccgacgagtccgcagagaactgcgacaagtccctgcataccctgtttggcgacaagctgtgtacagtggccaccctgcgggagacatacggtgagatggccgactgctgcgccaagcaggaacccgagagaaacgaatgcttcctgcagcacaaggacgacaaccccaacctgccccggctggtgaggcccgaagtggacgtgatgtgtaccgcctttcacgacaacgaagagacatttctgaagaagtacctgtacgagatcgccagaaggcacccttacttttacgcccccgagctgctgttctttgccaagaggtataaggccgcctttactgagtgctgccaggcagccgacaaagccgcctgcctgctgcccaagctggacgagctgcgcgacgagggcaaggcctcctccgctaagcagaggctgaagtgcgcatccctgcagaaatttggcgagagagccttcaaggcatgggccgtggctaggctgtcccagaggtttccaaaggccgaatttgccgaggtgtccaagctggtgacagacctgaccaaggtgcacaccgagtgttgtcacggagacttgctggagtgcgccgacgacagggccgacctggctaagtacatctgtgagaaccaggactctatctcctccaagctgaaggagtgctgcgagaagcccctgctggagaaaagccactgtattgctgaggtggagaacgacgagatgcccgccgacctgccctctctggccgctgatttcgtggagtccaaggacgtgtgtaagaactacgccgaggccaaagacgtgttcctggggatgtttctgtatgagtacgctaggaggcaccccgattactccgtggtgctgctgctgaggctggcaaaaacatacgaaaccaccctggagaagtgctgtgccgctgctgacccccacgagtgctatgccaaggtgttcgacgagttcaaacccctggtggaggaaccccagaacctgatcaagcagaactgcgagctgttcgaacagctgggcgagtacaaattccagaacgcactgctggtgaggtacaccaagaaagtgccccaggtgagcacccccaccctggtggaggtgtccaggaacctgggcaaagtgggcagcaaatgctgcaagcaccccgaggccaaaagaatgccctgtgcagaggactacctgagtgtggtgctgaaccagctgtgcgtgctgcacgagaagacacccgtgagcgacagggtgaccaagtgttgtaccgagtccctggtgaacagaaggccctgttttagtgccctggaagtggatgagacttacgtgcctaaggagttcaacgcagagacattcaccttccatgccgatatttgcaccctgtccgaaaaggaaagacagatcaagaaacagaccgccctggtggagctggtgaaacacaagcccaaggccaccaaggaacagctgaaagccgtgatggacgacttcgccgccttcgtggagaaatgctgcaaggccgacgacaaagaaacctgcttcgcagaggaaggaaagaaactggtggcagccagccaggccgccctgggactgggagatgacgacgacaagtactttggaaagctggagagcaagctgagtgtgatcaggaacctgaacgaccaggtgctgtttatcgaccagggcaataggccactgtttgaggatatgacagattccgacgtgagggacaacgctcccagaacaattttcatcatctctaagtacggagactcccagccaaggggcctggctgtgaccatctccgtgaaagtggagaaaatctccacactgtccgtggagaacaagatcatctccttcaaggaaatgaacccccccgacaacatcaaggacaccaaatccgacatcatcttcttccagcgctccgtgcccggacatgataacaaggtccagttcgagtccagcagctacgagggatactttctggccgtcgaaaaggaaagggacctcttcaaactgatcctgaagaaggaggacgagctgggggacagaagcattatgttcaccgtgcagaacgaggac

SEQ ID NO: 119 - DNA, 1275 bp, synthetic construct
  source 1..1275: mol_type = other DNA
gagcctaaatccgcagacaaaacacacacatgccccccctgccccgcccctgaactgttgggaggacccagcgtgtttctgttcccccccaaacccaaggacaccctgatgatctccaggacccccgaggtgacctgcgtggtcgtggacgtctcccacgaggaccccgaggtgaagtttaattggtacgtggacggcgttgaggtgcacaacgccaagaccaaacccagggaggagcagtacaacagtacatacagggtggtgtccgtgctgaccgtgctgcaccaggactggctgaacggcaaggagtacaagtgcaaggtgtccaacaaagcactccccgcaccaattgaaaaaaccatctccaaagccaaaggacagcccagggaaccacaggtctacaccctgcccccctccagggaggaaatgaccaagaaccaggtcagcttaacttgcctggttaaaggcttctacccctccgacatcgctgtggagtgggagtccaacggccagcccgagaacaactacaaaaccacaccacccgtgctcgactccgacggctccttcttcctctactccaaactgacagtggacaagtccaggtggcagcagggcaacgtcttttcctgctccgtgatgcacgaggccctgcacaaccactatacccagaagtccctgtccctgtcccccggcaaagccgccgagcctgtggaggacaacgtgatcaactttgtggccatgaagttcatcgacaacacactgtacttcatcgcagaggacgacgagaacgacgacgacgacaagtacttcggaaaactggagtccaagctgtccgtgatcaggaacctgaatgatcaggtgctcttcatcgaccagggcaacaggcccctgtttgaggatatgaccgacagcgacgtgagggacaacgcacccaggacaattttcatcatttctatgtacaaggactcccagcccaggggcatggccgtgaccatcagcgtgaaggtggagaagatcagcacactgagcgtggagaacaagatcatctccttcaaggagatgaacccccccgacaacatcaaagacaccaaaagcgatatcatcttctttcagaggagcgtgcccggccacgacaacaaaatgcagttcgagtcctcctcctacgagggctacttcctcgccgtcgaaaaggaaagggacctgttcaagctgatcctgaagaaggaggacgagctgggggacagaagcatcatgttcaccgtgcagaacgaggac

SEQ ID NO: 120 - DNA, 1182 bp, synthetic construct
  source 1..1182: mol_type = other DNA
gagcctaaatccgcagacaaaacacacacatgccccccctgccccgcccctgaactgttgggaggacccagcgtgtttctgttcccccccaaacccaaggacaccctgatgatctccaggacccccgaggtgacctgcgtggtcgtggacgtctcccacgaggaccccgaggtgaagtttaattggtacgtggacggcgttgaggtgcacaacgccaagaccaaacccagggaggagcagtacaacagtacatacagggtggtgtccgtgctgaccgtgctgcaccaggactggctgaacggcaaggagtacaagtgcaaggtgtccaacaaagcactccccgcaccaattgaaaaaaccatctccaaagccaaaggacagcccagggaaccacaggtctacaccctgcccccctccagggaggaaatgaccaagaaccaggtcagcttaacttgcctggttaaaggcttctacccctccgacatcgctgtggagtgggagtccaacggccagcccgagaacaactacaaaaccacaccacccgtgctcgactccgacggctccttcttcctctactccaaactgacagtggacaagtccaggtggcagcagggcaacgtcttttcctgctccgtgatgcacgaggccctgcacaaccactatacccagaagtccctgtccctgtcccccggcaaagacgacgacgataagtacttcggcaaactcgagagcaagttgtccgtgattcgcaacctgaacgaccaggtcctcttcatcgaccagggcaaccgccccctgttcgaggacatgaccgactccgacgtgagggacaacgctccccgcaccatcttcattatcagtatgtataaggattcccagccccgcggcatggccgtgaccatctccgtgaaggtggagaagatttccacattgtccgtggagaacaagatcatctccttcaaagagatgaacccccccgacaacatcaaggacacaaaatccgacatcatcttcttccagaggagtgtgcccggccacgacaacaaaatgcagttcgaatccagctcctacgagggctacttcctcgccgtcgaaaaggaaagggacctgttcaagctgatcctgaagaaggaggatgagctgggggacagatccatcatgttcaccgtgcagaacgaggac

SEQ ID NO: 121 - DNA, 1266 bp, synthetic construct
  source 1..1266: mol_type = other DNA
gagtctaagtacgggcccccatgcccctcctgccccgctcctgaatttttgggcggcccctccgtttttttgtttccccccaagcccaaggacaccctgatgatttcccgcacacccgaggtcacctgcgtcgtcgtcgacgtctcccaggaagatcccgaggtccagtttaattggtacgtggacggcgtggaagtccacaacgccaagaccaagccccgcgaggaacagttcaattcaacctacagggtggtgtccgtcctcaccgtgctgcatcaggattggctgaacggcaaggaatacaagtgcaaggtgtccaacaaaggcctcccctcctctatcgagaaaaccatctccaaagccaaaggccagcccagggaaccccaggtgtacaccctgcccccctcccaggaagaaatgaccaagaaccaggtgagcctgacatgcctcgtgaagggcttctacccctccgacatcgccgtggaatgggagtccaacggccagcccgaaaacaactacaaaaccacaccccccgtgctcgactccgacggctccttcttcctctactctaggttgaccgtggacaagtccaggtggcaggagggcaacgtcttttcctgctccgtgatgcacgaggccctccacaaccactatacccagaagtccctgtccctgtccctcggcaaggcagccgaacctgtggaggacaacgtgatcaacttcgtggccatgaagttcatcgataacaccctgtacttcatcgcagaagatgatgaaaacgacgacgacgacaaatacttcggcaaactggagtccaagctgagcgtgattaggaacctgaatgatcaggtgctcttcattgaccagggcaacaggcccctctttgaggacatgaccgactccgacgtgagggacaacgccccccgcaccatcttcatcatctccatgtacaaggactcccagcccaggggcatggccgtgaccatttccgtgaaggtggagaaaatctccacactgtctgtggagaacaaaatcatctccttcaaagagatgaacccccccgacaacatcaaagacacaaaaagcgatatcattttcttccagaggtccgtgcccggccacgacaacaaaatgcagttcgaatcctccagctacgagggctacttcctggccgtcgaaaaggaaagggacctgttcaagctcattctgaagaaggaggacgagctcggtgacaggtccatcatgttcaccgtccagaacgaggac

SEQ ID NO: 122 - DNA, 1173 bp, synthetic construct
  source 1..1173: mol_type = other DNA
gagtctaagtacgggcccccatgcccctcctgccccgctcctgaatttttgggcggcccctccgtttttttgtttccccccaagcccaaggacaccctgatgatttcccgcacacccgaggtcacctgcgtcgtcgtcgacgtctcccaggaagatcccgaggtccagtttaattggtacgtggacggcgtggaagtccacaacgccaagaccaagccccgcgaggaacagttcaattcaacctacagggtggtgtccgtcctcaccgtgctgcatcaggattggctgaacggcaaggaatacaagtgcaaggtgtccaacaaaggcctcccctcctctatcgagaaaaccatctccaaagccaaaggccagcccagggaaccccaggtgtacaccctgcccccctcccaggaagaaatgaccaagaaccaggtgagcctgacatgcctcgtgaagggcttctacccctccgacatcgccgtggaatgggagtccaacggccagcccgaaaacaactacaaaaccacaccccccgtgctcgactccgacggctccttcttcctctactctaggttgaccgtggacaagtccaggtggcaggagggcaacgtcttttcctgctccgtgatgcacgaggccctccacaaccactatacccagaagtccctgtccctgtccctcggcaaggacgacgacgataagtacttcggcaaactggagagcaagctgtccgtgatccgcaatctgaacgatcaggtcctcttcatcgaccagggcaaccgccccctgttcgaggacatgaccgacagcgacgtgagggacaacgcccccaggacaatcttcatcatttccatgtacaaggatagccagcccaggggcatggcagtgaccatctccgtgaaagtggagaagatcagcaccttgagcgtggagaacaagatcatcagcttcaaggagatgaacccccccgacaacatcaaggacaccaaatccgacatcatcttcttccagcgctccgtgcctggccacgacaacaaaatgcagttcgagtccagcagctacgaaggctacttcctggccgtcgaaaaggaaagggacctgttcaagctgatcctgaagaaggaggacgagctgggggacaggagcatcatgttcaccgtccagaacgaagac

SEQ ID NO: 123 - DNA, 1347 bp, synthetic construct
  source 1..1347: mol_type = other DNA
caggtgcagctgaagcagtctggaccaggactcgtgcagccaagtcagtctctctccatcacatgcacagtctccggcttctccctcacaaactacggcgtccactgggtgcgacagtcacctggaaaaggcctcgaatggctcggcgtgatctggtccggaggaaataccgactacaacaccccctttacctccaggctgtccatcaacaaggataattccaagtcccaggtgttcttcaagatgaatagcctgcagtccaacgataccgccatctactactgcgccagggctctgacatactacgactacgagttcgcctactggggccagggaacactggtgaccgtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccgcttcacgcagaagagcctctccctgtctccgggtaaa

SEQ ID NO: 124 - DNA, 1350 bp, synthetic construct
  source 1..1350: mol_type = other DNA
gaagtgcagctggtggagagcggaggaggattggtgcagcctggaggaagcctgaggctgagttgtgccgctagtgggtttaatatcaaggacacatacatccactgggtgaggcaggcccccggaaaaggattggagtgggtggcacggatttaccccaccaatggatacacaaggtacgccgactccgtgaagggcaggttcaccatctccgctgacacaagcaagaacacagcctacctgcagatgaactccctgagggcagaagatactgctgtgtactattgctccaggtggggaggagacggattctatgcaatggactactggggccagggaacactggtgaccgtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccgcttcacgcagaagagcctctccctgtctccgggtaaa

SEQ ID NO: 125 - DNA, 696 bp, synthetic construct
  source 1..696: mol_type = other DNA
gaacctaaatcctccgacaaaacccacacctgccccccttgccctgctcctgaagctgctggaggaccttccgtgtttctgtttccccccaagcccaaggacaccctcatgatctccaggacccccgaggtcacctgcgttgttgtggacgtgtcccacgaagatcctgaagttaaatttaactggtacgtggacggcgtggaagtgcacaacgccaagacaaaaccccgcgaggaacagtataactccacctacagggtggtgagcgtgctgacagtgctgcaccaggactggctgaacggcaaggaatacaagtgcaaggtgtccaacaaggccctgggcgctcctattgagaaaactatttctaaagccaagggacagccccgggagccacaggtttacacactgcctccctccagggaagagatgaccaagaaccaggtcagcctgtcctgcgctgttaagggcttctacccctccgacatcgccgtggaatgggaatccaatggccagcccgagaacaactacaaaaccaccccccccgtgctggactccgatggatctttcttcctggtgtccaagctgaccgtggacaagtccagatggcagcagggcaatgtcttttcctgctccgtgatgcacgaggccctccataacagattcacccagaagtccctgtccctgtctcccggaaag

SEQ ID NO: 126 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaagccgccgagcccgtcgaggacaacgtgattaatttcgtggctatgaagttcatcgataacacactctattttatcgctgaggacgatgagaatattggcaacaggtcctatttcggcaagttggaaagcaagctgtcagtgatccgcaatctcaatgatcaggtgctgtttattgatcagggcaataggccactctttgaagatatgaccgattctgatgtccgcgacaatgcacctagaactatctttattatttccaagtatggggacagtcagcctcggggactggcagtcactatttccgtcaaggtggagaagattagcactctcagcgttgagaacaaaattatctctttcaaggagatgaacccaccagataacattaaggacacaaagtccgatatcatcttcttccagcgaagcgttccaggccatgataataaggtgcagttcgagtctagcagctatgagggctattttctggccgttgagaaggagcgagatttgtttaagctcatcctgaagaaggaggatgagttgggggatagaagtatcatgtttactgtccagaacgaggat

SEQ ID NO: 127 - DNA, 1317 bp, synthetic construct
  source 1..1317: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaagctgccgaacccgtggaggacaatgtgatcaattttgtggctatgaagtttattgataacaccctctactttatcgccgaagatgacgaaaatggaggcggcggcaagcctctgggcctgcaggctagagtcgttggcggaggaggtacatactttggcaaactggagtccaagctctctgtgattcggaacctgaatgatcaggtcttgttcatcgatcagggaaatcggcctttgtttgaagatatgaccgactccgacgtgcgagataacgccccaaggaccattttcatcatctccaaatatggtgactcccagcctcggggactcgcagtcaccatttcagtgaaggtggagaagatttctaccttgtctgttgagaataaaattatctcattcaaagagatgaatcctccagataatattaaagacacaaaatcagacatcattttcttccagcggtctgtgcctggccacgacaataaagtgcagttcgaatcctcttcttatgaggggtacttcctggctgtcgagaaggaaagggacctgtttaagctgattctgaagaaagaggatgagttgggtgaccgaagcattatgtttaccgtccagaatgaagat

SEQ ID NO: 128 - DNA, 1365 bp, synthetic construct
  source 1..1365: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaagccgccgagcccgtcgaagataacgtgatcaacttcgtggccatgaaattcatcgataatacactctactttattgccgaggatgacgagaacatcgagcaggatggcggctccatcggcaacagaagtggcggcggaggcaagcccttgggactgcaggccagggttgtcggaggtgggggtactattgagcaggattactttgggaagttggaatccaagctcagtgtgatccgcaatctgaatgatcaggtgctcttcatcgaccaggggaatcggcccctgttcgaagatatgaccgactccgatgttagggataacgctcctagaactatctttatcattagcaagtatggggactctcagccaagagggctggccgtgaccatctccgtcaaagtcgaaaagatttccactctctctgtggaaaataagatcatcagctttaaagagatgaatccccctgacaacatcaaggatacaaaatctgatatcatctttttccagcgctctgttcccggacatgacaacaaggtgcagtttgagtctagcagctacgaggggtatttcctcgctgttgaaaaggaacgagatctctttaaattgattttgaagaaggaagatgagctgggagacaggtcaatcatgttcaccgttcagaacgaagat

SEQ ID NO: 129 - DNA, 1329 bp, synthetic construct
  source 1..1329: mol_type = other DNA
caggttcagctcgtcgagtcaggcggaggagtcgttcagccaggaaggagtctcaggctggactgcaaggcatcaggcatcacattctccaactccggcatgcactgggtgaggcaggcacctggaaaaggattggagtgggtggcagtgatctggtacgatggaagcaaaagatactacgccgacagcgtcaagggcaggttcaccatcagcagggacaatagcaagaacaccctgttcttgcagatgaatagcttgagggccgaggacaccgccgtgtattactgcgctaccaacgacgactactggggccagggaaccctggttaccgtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 130 - DNA, 1992 bp, synthetic construct
  source 1..1992: mol_type = other DNA
caggttcagctcgtcgaaagcggaggaggagtcgttcagccaggaagatccctcaggctcgactgtaaggcctccggaatcaccttctccaactccggaatgcactgggtcaggcaggctcctggaaaaggactggagtgggtggctgtgatttggtacgacggctccaagagatactacgccgactctgtgaagggccggttcactatctccagggacaactccaagaacacactgttcctgcagatgaactccctgcgggctgaggatactgccgtttattactgcgccaccaacgatgactactggggacagggaaccttggtgaccgtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaacaggttcacacagaagagcttgagcttgagccccggcggaggcgggggatctgggggaggaggtagtggaggcggaggatccggcggtggaggttctggtggtgggggcagtgggggtggcggatcagctgctgaacctgtggaagataattctattaacttcgtggccatgaagttcatcgataatactctgtacttcatcgccgaagatgatgaaaatatcgagcaggattacttcggcaaactggaaagcaaactgagcgtgatcaggaacctcaacgaccaggtgctgttcatcgatcagggcaacaggcccctctttgaggacatgaccgacagcgatagcagggacaacgcacctaggacaatcttcatcatctccaagtacggcgactcccagcccagaggattggctgtgacaatttccgtgaaatccgagaagatctccaccctgtccagcgagaacaagatcatctccttcaaggagatgaacccccccgacaacatcaaggacacaaagtccgacatcatcttcttccagcgctccgtgcccggtcatgataacaaggtccagttcgagtccagctcctacgagggatactttctggcctcagagaaggaaagggacctgttcaaactcatcctgaagaaggaggacgagctgggggacagaagcatcatgttcaccgtccagaatgaggac

SEQ ID NO: 131 - DNA, 1329 bp, synthetic construct
  source 1..1329: mol_type = other DNA
caggttcagctcgtcgaaagcggaggaggagtcgttcagccaggaagatccctcaggctcgactgtaaggcctccggaatcaccttctccaactccggaatgcactgggtcaggcaggctcctggaaaaggactggagtgggtggctgtgatttggtacgacggctccaagagatactacgccgactctgtgaagggccggttcactatctccagggacaactccaagaacacactgttcctgcagatgagcagtttgagggccgaggacaccgctgtttactactgcgcaaccaacgatgactactgggggcagggaaccctggttacagtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaacaggttcacgcagaagagcctctccctgtctccgggtaaa

SEQ ID NO: 132 - DNA, 696 bp, synthetic construct
  source 1..696: mol_type = other DNA
gagcccaaatctagcgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 133 - DNA, 1302 bp, synthetic construct
  source 1..1302: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataattctattaattttgtggccatgaagttcattgataatactctgtatttcatcgccgaagatgatgagaacgacgacgacgataaatatttcggaaaactggagtccaagctgagcgtgatcaggaacctgaatgatcaggtgctgttcattgaccagggcaacaggcccctgtttgaggatatgaccgacagcgacagcagggataacgctccccggacaatcttcatcatctccaagtacggcgattcccagcccaggggactggccgtgaccatcagcgtgaaatctgagaagatctccaccctgagctccgaaaacaagatcatcagcttcaaggagatgaacccccccgacaacattaaggacaccaaatccgacatcatcttcttccagagatccgtgcccggccacgacaacaaagtgcagttcgagtcctcctcctacgagggctactttctggcctccgaaaaagaaagggacctgttcaagctgatcctgaagaaggaggacgaactgggggacaggagcatcatgttcaccgtgcagaacgaggacatcatgttcaccgtgcagaacgaggac

SEQ ID NO: 134 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgccattaattttgtggccatgaaattcatcgataacaccctgtactttattgccgaggatgatgaaaatgatgatgatgacaaatacttcggcaaactggaatccaagctgtccgtgatcaggaacctgaatgatcaggtgctgttcattgaccagggcaaccgccccctgttcgaggacatgaccgattccgatgccagggacaacgcaccccggaccatcttcatcatctctaagtacggagactcccagccaaggggcctggccgtgaccatctccgtgaaggccgagaaaatttccaccctgtccgccgaaaacaaaatcatctccttcaaagagatgaacccccccgacaacatcaaggacaccaaatccgacatcatcttcttccagcgctccgtgcccggccacgacaacaaagtgcagttcgagtccagctcctacgagggctacttcctggccgccgaaaaggaaagggacctgttcaagctgatcctgaagaaggaggacgagctgggtgacaggagcatcatgttcaccgtgcagaacgaggac

SEQ ID NO: 135 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaattttgtggccatgaaatttattgacaacaccctgtatttcatcgccgaagatgatgaaaacgatgatgatgataagtactttggaaaactggaatcaaagctgagcgtgatccggaacctgaatgatcaggtgctgttcatcgaccagggcaacaggcccctgtttgaggacatgaccgactccgacgtgagggacaacgctccccggaccattttcatcatctctaagtacggcgacagccagcccaggggactggccgtgaccatttccgtgaaggtggagaagatttccacactgtccgtggagaacaaaatcatctccttcaaagagatgaacccccccgacaacatcaaagacacaaaaagcgatattatcttcttccagcgctctgtgcccggccacgacaacaaggtgcagttcgagagctccagctacgagggctactttctggccgtggagaaggaaagggacctgttcaaactgattctgaagaaggaggacgaactgggggacaggtccatcatgttcaccgtgcagaatgaagac

SEQ ID NO: 136 - DNA, 1185 bp, synthetic construct
  source 1..1185: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggagatgacgacgacaagtactttggaaagctggagagcaagctgagtgtgatcaggaacctgaacgaccaggtgctgtttatcgaccagggcaataggccactgtttgaggatatgacagattccgacgtgagggacaacgctccccggaccatcttcattatcagtaagtatggcgactcccagccaaggggcctggctgtgaccatcagcgtgaaagtggagaagattagcacactgtccgtggagaacaagatcatctccttcaaggagatgaacccccccgacaacatcaaagacaccaagtccgacatcattttcttccagaggtccgtgcctggacacgacaacaaagtgcagttcgagtcctcctcctacgagggctacttcctggccgtggaaaaagagcgcgacctgttcaagctgatcctgaagaaggaggacgagctgggggacaggagcatcatgttcaccgtgcagaacgaggac

SEQ ID NO: 137 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaattttgtggccatgaaatttattgacaacaccctgtatttcatcgccgaagatgatgaaaacgatgatgatgataagtactttggaaaactggaatcaaagctgagcgtgatccggaacctgaatgatcaggtgctgttcatcgaccagggcaacaggcccctgtttgaggacatgaccgactccgacgtgagggacaacgctccccggaccattttcatcatctctatgtacaaggacagccagcccaggggtatggccgtgaccattagcgtgaaggtggagaagattagcacactgtccgtggagaacaaaatcatctccttcaaggaaatgaacccccccgacaacatcaaagacaccaagagcgacatcatcttcttccagaggagcgtgcccgggcacgacaacaaaatgcagttcgaatccagctcctacgagggctacttcctggccgtggagaaagagagggacctgttcaagctgatcctgaagaaggaggacgagctgggggacaggagcatcatgttcaccgtgcagaacgaggac

SEQ ID NO: 138 - DNA, 1272 bp, synthetic construct
  source 1..1272: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagctcctcctacgagggatactttctggccgtcgaaaaggagagggacctgttcaagctgatcctgaagaaggaggacgagctgggggacagatcaattatgttcaccgtgcagaatgaggac

SEQ ID NO: 139 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataattctattaacttcgtggccatgaagttcatcgataatactctgtacttcatcgccgaagatgatgaaaatgacgacgatgacaagtacttcggtaaactggagtccaagctgtccgtgatcaggaacctgaatgatcaggtgctgttcattgaccagggcaacaggcccctgtttgaggatatgaccgactccgactctagagataacgccccccgaacaatcttcatcatcagtatgtacaaggactcccagcccaggggaatggctgtgaccattagcgtgaagtccgagaagattagcaccctgtcctccgagaacaagatcatctccttcaaagagatgaacccccccgacaacatcaaagacaccaagagcgacatcatcttcttccagaggagcgtgcccggccatgataacaaaatgcagttcgaaagctcctcctacgagggctacttcctggcatccgaaaaggaaagggacctgttcaaactgatcctgaagaaggaggatgagctgggggacaggtccatcatgttcaccgtccagaacgaggac

SEQ ID NO: 140 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgccattaatttcgtggccatgaagttcattgacaacaccctgtacttcatcgccgaggatgatgagaacgacgacgacgataaatatttcggaaagctggagtccaagctgagcgtgatcaggaacctgaatgatcaggtgctgttcattgaccagggcaacaggcccctgtttgaggatatgaccgacagcgacgccagggataacgctcctagaacaatcttcatcatctctatgtacaaggactcccagcccaggggaatggctgtgaccatttccgtgaaggccgagaagatttccaccctgtcagccgaaaacaagatcatctccttcaaagagatgaacccccccgacaacatcaaagacaccaaatccgacatcatcttcttccagcgcagcgtgcccggacatgataacaaaatgcagttcgaaagctcctcctacgagggctacttcctcgcagcagaaaaagaaagggacctgttcaagctgatcctgaagaaggaggacgagctgggggacagaagcatcatgttcaccgtgcagaacgaagac

SEQ ID NO: 141 - DNA, 1275 bp, synthetic construct
  source 1..1275: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgtgatcaatttcgtggccatgaagttcattgacaacaccctgtacttcatcgctgaagatgatgaaaacgacgatgatgataagtacttcggaaagctggagtccaagctgagcgtgattaggaacctgaatgaccaggtgctgttcattgaccagggcaacaggcccctctttgaggatatgaccgactccgatgtgagggacaacgcccctagaacaatcttcatcatctccaagtacggcgactccggagcaaggggactggctgtgaccatttccgtgaaagtggagaagatttccaccctgtccgtggagaacaagatcatttccttcaaagagatgaaccccccagacaacattaaggacacaaagagcgatatcatcttcttcgagcgcgatgtgcccgggcattctggaaaagtgcagttcgagtcctcctcctacgagggttactttctggccgtcgaaaaggaaagggacctgttcaagctgatcctgaagaaggaggacgagctgggggacagatccatcatgttcaccgtgcagaatgaggac

SEQ ID NO: 142 - DNA, 1272 bp, synthetic construct
  source 1..1272: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaatttcgtggccatgaagttcattgacaacaccctgtacttcatcgccgaagatgatgaaaacatcgaacaggactacttcggaaaactggaatccaaactgagcgtgatcaggaacctgaacgaccaggtgctgttcatcgaccagggcaatcgccccctctttgaggacatgaccgactccgacgtccgggacaacgctcccaggaccatcttcatcatctccatgtacaaagactcccagccaaggggcatggccgtgaccatctccgtcaaggtggagaagatctccaccctgtccgtggaaaacaagatcatctccttcaaagagatgaacccccccgacaacatcaaagacacaaaatccgacatcatcttcttccagcgctccgtgcctgggcacgacaacaaaatgcagttcgaatcctcctcctacgagggctacttcctcgccgtcgaaaaagaaagagacttgttcaagctcatcctcaagaaggaggacgagctcggggacaggtcaatcatgttcaccgtgcagaacgaggac

SEQ ID NO: 143 - DNA, 1284 bp, synthetic construct
  source 1..1284: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaatttcgtggccatgaagttcattgacaacaccctgtacttcatcgccgaagatgatgaaaacggacccctgggagtgaggggaggatacttcggaaagctggagagcaagctgagcgtgatcaggaatctgaacgaccaggtgctgttcatcgaccagggcaacaggcccctgttcgaggacatgacagacagtgacgtgagggacaacgccccccggaccatcttcatcatcagtatgtacaaggactcccagccaaggggcatggccgtgaccatctccgtgaaagtggagaaaatcagcacactgagcgtggagaacaagatcatctccttcaaggagatgaacccccccgacaacatcaaagacacaaaatccgacatcatcttcttccagaggagcgtgcccggccacgacaacaaaatgcagttcgaatccagctcctacgagggctacttcctcgccgtcgaaaaggaaagggacctgttcaagctcatcctgaagaaggaggacgagctgggggacaggagcatcatgttcaccgtccagaacgaggac

SEQ ID NO: 144 - DNA, 1296 bp, synthetic construct
  source 1..1296: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaatttcgtggccatgaagttcattgacaacaccctgtacttcatcgccgaagatgatgaaaacgtgcatatgcctctgggctttctgggacccggaggctattttggaaagctggagagcaagctgtccgtgatccgcaacctgaacgaccaggtgctgttcatcgaccagggaaacaggcccctgttcgaggacatgaccgactctgacgtgagggacaacgcccccaggacaatcttcatcatctccatgtacaaggactcccagccaaggggcatggccgtgaccatctccgtgaaggtggagaagatctccaccctgagcgtggagaacaagatcatcagcttcaaggagatgaacccccccgacaacatcaaagacacaaaatccgacatcatcttcttccagaggagcgtgcctggccacgacaacaaaatgcagtttgagtcctcctcctacgagggctacttcctggccgtcgaaaaggaaagggacctgttcaagctgatcctgaagaaggaggacgagctgggcgacaggtcaattatgttcaccgtgcagaacgaggac

SEQ ID NO: 145 - DNA, 1272 bp, synthetic construct
  source 1..1272: mol_type = other DNA
gctgctgaacctgtggaagataatgtgatcaatttcgtggccatgaagttcattgacaacaccctgtacttcatcgctgaagatgatgaaaacctggagtccgactacttcggaaaactggaatccaaactgagcgtgatccggaacctgaacgatcaggtgctgttcatcgaccagggcaatcggcctctgtttgaggacatgaccgacagcgacgtgagggataacgctcctaggaccatcttcatcatctccatgtataaggactcccagccaaggggcatggccgtgaccatctccgtgaaagtggagaagatctccacactgtccgtggaaaacaagatcatctccttcaaagagatgaacccccccgacaacatcaaagacacaaaaagcgatattatcttcttccagcggagcgtgcccgggcacgacaacaagatgcagttcgaaagctcctcctacgagggatactttctggccgtcgaaaaggaaagggacctgttcaaactgattctgaagaaggaggacgagctgggggacagaagcatcatgttcaccgtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - DNA, 1167 bp, synthetic construct
  source 1..1167: mol_type = other DNA
tacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctccatgtacaaggactcccagcccaggggaatggctgtgaccatttccgtgaaagtggagaagattagcaccctgagcgtggagaacaagatcatctccttcaaggaaatgaacccccccgacaacatcaaagacaccaaatccgacatcatcttcttccagaggtccgtgcctggccacgataacaaaatgcagttcgagtcctcctcctacgagggctacttcctggcagtcgaaaaggagagggacctgttcaagctcatcctgaagaaggaggacgagctgggggacagatccatcatgttcaccgtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - DNA, 1182 bp, synthetic construct
  source 1..1182: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagacgatgacgacaagtatttcggaaagctggaaagcaagctgagcgtgatcaggaacctgaacgaccaggtgctgttcatcgaccagggaaacaggcccctgtttgaggacatgaccgacagcgacgtgagggataacgcccctagaacaatcttcattatctccatgtataaagactcccagccaaggggaatggccgtgaccatttccgtgaaggtcgagaaaatctccacactgagcgtggagaacaagatcatctccttcaaagagatgaacccccccgacaacatcaaagacacaaagagcgacatcatcttcttccagaggagcgtgcccggccatgataacaagatgcagttcgaaagctcctcctacgaaggctacttcctggccgtcgaaaaggaaagggacctgttcaaactgatcctcaagaaggaggacgagctgggggacagaagcatcatgttcaccgtgcagaacgaggac

SEQ ID NO: 148 - DNA, 1653 bp, synthetic construct
  source 1..1653: mol_type = other DNA
gaggtgcagctggtggagtccggaggaggactggtgcaggctggaggaagcctgaggctgtcctgcgctgcttccggaaggacctttagttcctatgctatgggttggtttaggcaggcccctggaaaggagagggagtttgtggtggccattaactggagtagcggaagcacctactacgccgacagtgtgaagggacggttcacaatctccagggacaatgctaagaacacaatgtatctgcagatgaacagcctgaagcctgaggacaccgccgtgtattactgcgcagctggataccagatcaatagcggaaactacaacttcaaggactacgaatacgactactggggccagggcacacaggtgaccgtgtcctccgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 149 - DNA, 1923 bp, synthetic construct
  source 1..1923: mol_type = other DNA
caggtgcagctgaagcagtctggaccaggactcgtgcagccaagtcagtctctctccatcacatgcacagtctccggcttctccctcacaaactacggcgtccactgggtgcgacagtcacctggaaaaggcctcgaatggctcggcgtgatctggtccggaggaaataccgactacaacaccccctttacctccaggctgtccatcaacaaggataattccaagtcccaggtgttcttcaagatgaatagcctgcagtccaacgataccgccatctactactgcgccagggctctgacatactacgactacgagttcgcctactggggccagggaacactggtgaccgtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 150 - DNA, 1167 bp, synthetic construct
  source 1..1167: mol_type = other DNA
tacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctccaagtatggagactcccagccaaggggactcgctgtgaccatttccgtgaaggtggagaagatcagcacactctccgtggaaaacaagatcatcagcttcaaagagatgaacccccccgacaacatcaaggacaccaaatccgacatcatcttcttccagcgctccgtgcctggccatgataacaaagtgcagttcgagtccagctcctacgagggatactttctcgccgtcgaaaaggaaagggacctgttcaagctcatcctgaagaaggaggacgaactgggcgacaggtccattatgttcaccgtccagaatgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 151 - DNA, 1167 bp, synthetic construct
  source 1..1167: mol_type = other DNA
tacttcggcaagctgaagtccaagctgtccgtgatccggaacctgaacgaccaggtgctgttcatcgaccagggcaacaggccactcttcgaggatatgaccgactccgacgtgagggacaacgctcctagaaccatcttcatcatctccatgtacggagactcccagccaaggggaatggctgtgacaatttccgtgaaggtggagaaaatttccacactgtccgtggaaaacaagatcatttccttcaaggagatgaacccccccgacaacattaaagacaccaagagcgacatcatcttcttccagcgctctgtgcccggccatgataacaagatgcagttcgagtccagctcctacgagggctactttctggccgtcgaaaaggaaagggacctgttcaaactgatcctgaagaaggaggacgaactgggggacaggagcatcatgttcaccgtgcagaatgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - DNA, 1167 bp, synthetic construct
  source 1..1167: mol_type = other DNA
tacttcggcaagctgcgttccaagctgtccgtgatccggaacctgaacgaccaggtgctgttcatcgaccagggcaacaggcctctcttcgaggatatgaccgactccgacgtgagggacaacgctcctagaaccatcttcatcatctccatgtacggagactcccagccaaggggaatggctgtgacaatttccgtgaaggtggagaaaatttccacactgtccgtggaaaacaagatcatttccttcaaggagatgaacccccccgacaacattaaagacaccaagagcgacatcatcttcttccagcgctctgtgcccggccatgataacaagatgcagttcgagtccagctcctacgagggctactttctggccgtcgaaaaggaaagggacctgttcaaactgatcctgaagaaggaggacgaactgggggacaggagcatcatgttcaccgtgcagaatgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 153 - DNA, 1983 bp, synthetic construct
  source 1..1983: mol_type = other DNA
caggtgcagctgaagcagtctggaccaggactcgtgcagccaagtcagtctctctccatcacatgcacagtctccggcttctccctcacaaactacggcgtccactgggtgcgacagtcacctggaaaaggcctcgaatggctcggcgtgatctggtccggaggaaataccgactacaacaccccctttacctccaggctgtccatcaacaaggataattccaagtcccaggtgttcttcaagatgaatagcctgcagtccaacgataccgccatctactactgcgccagggctctgacatactacgactacgagttcgcctactggggccagggaacactggtgaccgtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggagggtctggtggtggaggatccgggggaggaggttcaggaggcgggggaagtgctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 154 - DNA, 1077 bp, synthetic construct
  source 1..1077: mol_type = other DNA
gaagtgcagctggtggaaagcggaggaggattggtgcaggcaggaggaagtctgaggctgagttgtgccgctagcggtagaacatttagtagttatgctatggggtggtttaggcaggctcctggaaaggagagggagttcgtggtggctatcaactggagcagcggctctacatactacgctgattccgtgaagggaaggttcacaatctccagggacaatgctaagaacacaatgtatctgcagatgaacagcctcaagcctgaggacaccgccgtttactattgcgccgctggataccaaatcaacagcggcaactacaacttcaaggactatgagtatgactactggggccagggcacacaggtgacagtttcatccgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - DNA, 1167 bp, synthetic construct
  source 1..1167: mol_type = other DNA
tacttcggcaagctggcttccaagctgtccgtgatcaggaacctgaacgatcaggtgctgttcatcgatcagggcaacaggcccctgttcgaggatatgaccgactccgatgtgcgcgacaacgcacctagaaccatcttcatcatctctatgtacgccgattcccagccaaggggcatggctgtgacaatttccgtgaaggtggagaagatctccaccctgtccgtggaaaacaaaatcatttccttcaaggagatgaacccccccgacaacatcaaggacactaaaagcgacatcatcttcttccagaggagcgtgcccggccatgataacaaaatgcagttcgaatccagctcctacgaaggctacttcctcgccgtcgaaaaggaaagggacctgttcaaactgatcctcaagaaggaggacgagttgggggacagatccattatgttcaccgttcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 156 - DNA, 1167 bp, synthetic construct
  source 1..1167: mol_type = other DNA
tacttcggcaagctgcactccaagctgtccgtgatccggaacctgaacgaccaggtgctcttcatcgaccagggcaacaggcctctcttcgaggatatgaccgactccgacgtgcgcgataacgctcctagaaccatcttcatcatctccatgtacggcgactcccagcccagaggaatggctgtgacaatttccgtgaaggtggagaaaatctccaccctgtccgtggagaacaagatcatctccttcaaagagatgaacccccccgacaacatcaaagacaccaagagcgacatcatctttttccagcgctctgtccccggccacgataataaaatgcagttcgaatccagctcctacgaaggctacttcctcgccgtcgaaaaggaaagggacctcttcaagttgatcctgaagaaggaggacgaactgggggacaggagcatcatgttcaccgtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 157 - DNA, 1167 bp, synthetic construct
  source 1..1167: mol_type = other DNA
tacttcggcaagctgcgttccaagctgtccgtgatccggaacctgaacgaccaggtgctgttcatcgaccagggcaacaggcctctcttcgaggatatgaccgactccgacgtgagggacaacgctcctagaaccatcttcatcatctccatgtacgaagattctcaacccaggggcatggccgtgacaatttccgttaaggtggagaaaatctccacactgtccgtggaaaacaagatcatctccttcaaagagatgaacccccccgacaacattaaagacacaaaaagcgatatcatcttcttccagaggagcgtgcccgggcatgataacaagatgcagttcgaaagctcctcctacgaaggctacttcctggccgtcgaaaaggaaagggacctgttcaaactgatcctgaagaaggaggatgaattgggcgacaggtccattatgttcacagtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 158 - DNA, 1167 bp, synthetic construct
  source 1..1167: mol_type = other DNA
tacttcggcaagctgcagtccaagctgtccgtgatccggaacctgaacgaccaggtgctgttcatcgaccagggcaacaggcctctcttcgaggatatgaccgactccgacgtgagggataacgctccaaggaccatcttcatcatctccatgtacgccgactcccagccaaggggaatggctgtgacaatctccgtgaaagtggagaagattagcaccctgtccgtggaaaacaagatcatctccttcaaagagatgaacccccccgacaacatcaaagacaccaaatccgacatcatcttcttccagcgctctgtgcctgggcacgataacaaaatgcagttcgaaagctcctcctacgagggatacttcctcgccgtcgaaaaggaaagggacctgttcaagctgatcctgaagaaggaggacgagctgggggacagaagcatcatgttcaccgttcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 159 - DNA, 1329 bp, synthetic construct
  source 1..1329: mol_type = other DNA
gagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacccgtggaagataatgtgatcaatttcgtggccatgaagttcattgacaacaccctgtacttcatcgccgaagatgatgaaaacggaggaggaggaaagccactgggattgcaggccagggtggtgggaggaggaggcacaatcgaacaggactacttcggaaaactggaatccaaactgagcgtgatcaggaacctgaacgaccaggtgctgttcatcgaccagggcaatcgccccctctttgaggacatgaccgactccgacgtccgggacaacgctcccaggaccatcttcatcatctccatgtacaaagactcccagccaaggggcatggccgtgaccatctccgtcaaggtggagaagatctccaccctgtccgtggaaaacaagatcatctccttcaaagagatgaacccccccgacaacatcaaagacacaaaatccgacatcatcttcttccagcgctccgtgcctgggcacgacaacaaaatgcagttcgaatcctcctcctacgagggctacttcctcgccgtcgaaaaagaaagagacttgttcaagctcatcctcaagaaggaggacgagctcggggacaggtcaatcatgttcaccgtgcagaacgaggac

SEQ ID NO: 160 - DNA, 1167 bp, synthetic construct
  source 1..1167: mol_type = other DNA
tacttcggcaagctgaagtccaagctgtccgtgatccggaacctgaacgaccaggtgctgttcatcgaccagggcaacaggccactcttcgaggatatgaccgactccgacgtgagggacaacgctcctagaaccatcttcatcatctcccactacggcgactcccagccaagaggaatggctgttacaatttccgtgaaagtggagaaaatctccaccctgtccgttgagaacaagatcatctccttcaaggagatgaacccccccgacaacatcaaggacaccaagagcgacatcatcttcttccagcgctctgtgcccggccatgataacaaaatgcagttcgaaagctcctcctacgagggctacttcctcgcagtcgaaaaagaaagggacctgttcaagttgatcctgaagaaggaggacgaactgggggacaggagcatcatgttcaccgtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - DNA, 1167 bp, synthetic construct
  source 1..1167: mol_type = other DNA
tacttcggcaagctgaagtccaagctgtccgtgatccggaacctgaacgaccaggtgctgttcatcgaccagggcaacaggccactcttcgaggatatgaccgactccgacgtgagggacaacgctcctagaaccatcttcatcatctccaagtacggagactcccagccaaggggaatggctgttacaatttccgtgaaggtggagaaaatctccaccctgtccgtggagaacaagatcatcagcttcaaagagatgaacccccccgacaacatcaaagacaccaagagcgacatcatcttcttccagcgctctgtgcccggtcacgataacaaaatgcagttcgaatccagctcctacgagggctacttcctcgcagtcgaaaaggaaagggacctcttcaagttgatcctgaagaaggaggacgagctgggggacagatccatcatgttcaccgttcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 162 - DNA, 1167 bp, synthetic construct
  source 1..1167: mol_type = other DNA
tacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctcccactacgccgactcccagccaagaggaatcgctgttaccatctccgtgaaggtggagaaaattagcacactgagcgtggagaacaagatcatctccttcaaggagatgaacccccccgacaacatcaaggacaccaaaagcgacatcatcttcttccagcgcagcgtgcccggacatgataacaaaatccagttcgaatcctccagctacgagggctactttctggccgtcgaaaaggaaagagacttgttcaagttgatcctgaagaaggaggacgagctgggggacagaagcatcatgttcaccgttcagaacgaagacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 163 - DNA, 1167 bp, synthetic construct
  source 1..1167: mol_type = other DNA
tacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctccaagtatgccgactcccagccaaggggaatcgctgtgacaatttccgtgaaagtggagaagatctccaccctgtccgtggaaaacaagatcatctccttcaaagagatgaacccccccgacaacatcaaggacaccaaatccgacatcatcttcttccagcgctctgtgcctgggcacgataacaaaatccagttcgaatccagctcctacgagggctactttctcgccgtcgaaaaggaaagggacctgttcaaactcatcctgaagaaggaggacgaactgggagacagatccatcatgttcacagtgcagaacgaggacgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 164 - DNA, 2040 bp, synthetic construct
  source 1..2040: mol_type = other DNA
caggtgcagctgaagcagtctggaccaggactcgtgcagccaagtcagtctctctccatcacatgcacagtctccggcttctccctcacaaactacggcgtccactgggtgcgacagtcacctggaaaaggcctcgaatggctcggcgtgatctggtccggaggaaataccgactacaacaccccctttacctccaggctgtccatcaacaaggataattccaagtcccaggtgttcttcaagatgaatagcctgcagtccaacgataccgccatctactactgcgccagggctctgacatactacgactacgagttcgcctactggggccagggaacactggtgaccgtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggagggtctggtggtggaggatccgggggaggaggttcaggaggcgggggaagtgctgctgaacctgtggaagataatgtgatcaattttgtggccatgaaatttattgacaacaccctgtatttcatcgccgaagatgatgaaaacggcggaggaggaaaacccctgggactgcaggctagagtggtgggaggaggaggaaccatcgagcaggactacttcgggaaactggaatctaagctgtcagttatcaggaacctgaacgatcaggtgctgttcattgaccagggcaacaggcccttgtttgaggatatgacagacagcgacgtgagggacaacgctcccagaaccatcttcattatctccaagtacggagacagccagcccaggggattggctgttaccatctccgtgaaagtggagaagatttccaccttgtcagtggagaacaaaatcatctccttcaaagagatgaacccccccgacaacatcaaggacaccaagtccgacatcatcttcttccagcgctctgtgcctgggcacgacaacaaagtccagttcgagagctccagctacgagggctactttctggccgtcgagaaggaacgcgacctgttcaaactcatcctgaaaaaggaggacgaactgggagacaggtccatcatgttcaccgtgcagaatgaagac

SEQ ID NO: 165 - DNA, 2043 bp, synthetic construct
  source 1..2043: mol_type = other DNA
gaagtgcagctggtggagagcggaggaggattggtgcagcctggaggaagcctgaggctgagttgtgccgctagtgggtttaatatcaaggacacatacatccactgggtgaggcaggcccccggaaaaggattggagtgggtggcacggatttaccccaccaatggatacacaaggtacgccgactccgtgaagggcaggttcaccatctccgctgacacaagcaagaacacagcctacctgcagatgaactccctgagggcagaagatactgctgtgtactattgctccaggtggggaggagacggattctatgcaatggactactggggccagggaacactggtgaccgtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggagggtctggtggtggaggatccgggggaggaggttcaggaggcgggggaagtgctgctgaacctgtggaagataatgtgatcaattttgtggccatgaaatttattgacaacaccctgtatttcatcgccgaagatgatgaaaacggcggaggaggaaaacccctgggactgcaggctagagtggtgggaggaggaggaaccatcgagcaggactacttcgggaaactggaatctaagctgtcagttatcaggaacctgaacgatcaggtgctgttcattgaccagggcaacaggcccttgtttgaggatatgacagacagcgacgtgagggacaacgctcccagaaccatcttcattatctccaagtacggagacagccagcccaggggattggctgttaccatctccgtgaaagtggagaagatttccaccttgtcagtggagaacaaaatcatctccttcaaagagatgaacccccccgacaacatcaaggacaccaagtccgacatcatcttcttccagcgctctgtgcctgggcacgacaacaaagtccagttcgagagctccagctacgagggctactttctggccgtcgagaaggaacgcgacctgttcaaactcatcctgaaaaaggaggacgaactgggagacaggtccatcatgttcaccgtgcagaatgaagac

SEQ ID NO: 166 - DNA, 1653 bp, synthetic construct
  source 1..1653: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgaacagcctgaagcctgaggacaccgccgtgtactattgcgccgctggatatcagatcaattccggcaactacaacttcaaggactacgagtatgactactggggccagggcacacaggtgacagtgtcatccgaacctaaatcctccgacaaaacccacacctgccccccttgccctgctcctgaagctgctggaggaccttccgtgtttctgtttccccccaagcccaaggacaccctcatgatctccaggacccccgaggtcacctgcgttgttgtggacgtgtcccacgaagatcctgaagttaaatttaactggtacgtggacggcgtggaagtgcacaacgccaagacaaaaccccgcgaggaacagtataactccacctacagggtggtgagcgtgctgacagtgctgcaccaggactggctgaacggcaaggaatacaagtgcaaggtgtccaacaaggccctgggcgctcctattgagaaaactatttctaaagccaagggacagccccgggagccacaggtttacacactgcctccctccagggaagagatgaccaagaaccaggtcagcctgtggtgcctggtgaagggattctacccctccgacatcgccgtggaatgggaaagcaacggccagcctgagaacaactacaaaaccacccccccagtgctggactccgacggatctttcttcctgtactctaagttgaccgtggacaagtccaggtggcagcagggaaacgtgttctcctgctctgtgatgcacgaggccctgcacaaccactatacccagaagagtttgtccctgtcccccggcaaggctgctgaacctgtggaagataatgtgatcaattttgtggccatgaaatttattgacaacaccctgtatttcatcgccgaagatgatgaaaacattgaacaggactatttcggaaagctggaatccaaattgagcgtgatccggaacctgaacgatcaggtgctgttcatcgaccagggcaacaggcctctgttcgaggacatgaccgactccgacgtgagggacaacgcccctagaacaatcttcatcatctccaagtacggagactcccagccaaggggcctggctgttacaatctccgtgaaggtggagaagatctccaccctgtccgtggagaacaagatcatctccttcaaggagatgaacccccccgacaacatcaaggacaccaaatccgacatcatcttcttccagcgctccgtgcctgggcacgataacaaagtccagttcgagtccagctcctacgagggctacttcctggccgtcgaaaaggaaagggacctgttcaagctgatcctgaagaaggaggacgagctgggagacaggagcatcatgttcaccgtccagaacgaggac

SEQ ID NO: 167 - DNA, 1653 bp, synthetic construct
  source 1..1653: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgaacagcctgaagcctgaggacaccgccgtgtactattgcgccgctggatatcagatcaattccggcaactacaacttcaaggactacgagtatgactactggggccagggcacacaggtgacagtgtcatccgaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataattctattaatttcgtggccatgaaattcatcgataatactctgtacttcatcgccgaagatgatgaaaatattgaacaggactatttcggaaagctggaaagcaaactgagcgtgatcaggaacctgaacgatcaggtgctgttcatcgaccagggcaaccgccctctgttcgaggatatgaccgactccgacagcagggacaacgcaccaaggaccatcttcatcatctccaagtacggcgactcccagccaaggggactggctgttaccatctccgtgaagtccgagaagatttccaccctgagctccgagaacaagatcatttccttcaaggagatgaacccccccgacaacatcaaagacaccaaatccgacatcatcttcttccagcgcagcgtgcccggacacgataacaaagtgcagtttgagtccagctcctacgagggctacttcctggcctccgaaaaggaaagggacctgttcaagctgatcctgaagaaggaggacgagttgggggacaggtcaattatgttcaccgttcagaacgaggac

SEQ ID NO: 168 - DNA, 1653 bp, synthetic construct
  source 1..1653: mol_type = other DNA
gaagtgcagctggtggaaagcggaggaggattggtgcaggcaggaggaagtctgaggctgagttgtgccgctagcggtagaacatttagtagttatgctatggggtggtttaggcaggctcctggaaaggagagggagttcgtggtggctatcaactggagcagcggctctacatactacgctgattccgtgaagggaaggttcacaatctccagggacaatgctaagaacacaatgtatctgcagatgaacagcctcaagcctgaggacaccgccgtttactattgcgccgctggataccaaatcaacagcggcaactacaacttcaaggactatgagtatgactactggggccagggcacacaggtgacagtttcatccgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataattctattaatttcgtggccatgaaattcatcgataatactctgtacttcatcgccgaaaatgatgaaaaccaggacattgaatactttggaaaactggaatccaaattgagtgtgatccgcaacctgaacgatcaggtgctgttcatcgaccagggcaatcgccccctgttcgaggatatgaccgactccgactccagggacaacgcacctaggaccatcttcatcatctctaagtacggcgattcccagcccaggggcttggctgttaccatctccgttaagtccgagaagatttccaccctgtcctccgagaacaaaatcatctccttcaaagaaatgaacccccccgacaacatcaaggacaccaaaagcgatatcatcttcttccagaggtccgtgcccggccacgataacaaagtgcagttcgagtccagctcctacgagggctacttcctggcctccgaaaaggaacgcgacctgttcaagctgatcctgaagaaggaggacgagctgggggacagatccattatgttcaccgttcagaacgaggac

SEQ ID NO: 169 - DNA, 1329 bp, synthetic construct
  source 1..1329: mol_type = other DNA
caggttcagctcgtcgaaagcggaggaggagtcgttcagccaggaagatccctcaggctcgactgtaaggcctccggaatcaccttctccaactccggaatgcactgggtcaggcaggctcctggaaaaggactggagtgggtggctgtgatttggtacgacggctccaagagatactacgccgactctgtgaagggccggttcactatctccagggacaactccaagaacacactgttcctgcagatgaactccctgcgggctgaggatactgccgtttattactgcgccaccaacgatgactactggggacagggaaccttggtgaccgtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 170 - DNA, 1272 bp, synthetic construct
  source 1..1272: mol_type = other DNA
ggaggcgggggatctgggggaggaggtagtggaggcggaggatccggcggtggaggttctggtggtgggggcagtgggggtggcggatcaggaattgagcaggactactttggaaagctggagagcaagctgagcgtgatcaggaacctgaacgatcaggtgctgttcatcgatcagggaaacaggcctctgtttgaggacatgacagacagcgacgtcagggacaacgcacccagaaccatcttcatcatctccatgtacaaggactcccagcccaggggaatggccgttacaatttccgtgaaagtggagaagatctccacactgagcgtggagaacaagatcatctccttcaaggagatgaacccccccgacaacatcaaggacaccaaatccgacatcatcttcttccagcgctccgtgcccgggcatgataacaagatgcagttcgaaagctcctcctacgaaggctacttcctcgccgtcgaaaaggaaagggacctgttcaagctcatcctcaagaaggaggacgagctgggggacagaagcatcatgttcaccgtgcagaacgaagacgaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 171 - DNA, 1212 bp, synthetic construct
  source 1..1212: mol_type = other DNA
ggtggaggaggttctggaggtggtggatcaggaattgagcaggactactttggaaagctggagagcaagctgagcgtgatcaggaacctgaacgatcaggtgctgttcatcgatcagggaaacaggcctctgtttgaggacatgacagacagcgacgtcagggacaacgcacccagaaccatcttcatcatctccatgtacaaggactcccagcccaggggaatggccgttacaatttccgtgaaagtggagaagatctccacactgagcgtggagaacaagatcatctccttcaaggagatgaacccccccgacaacatcaaggacaccaaatccgacatcatcttcttccagcgctccgtgcccgggcatgataacaagatgcagttcgaaagctcctcctacgaaggctacttcctcgccgtcgaaaaggaaagggacctgttcaagctcatcctcaagaaggaggacgagctgggggacagaagcatcatgttcaccgtgcagaacgaagacgaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 172 - DNA, 1212 bp, synthetic construct
  source 1..1212: mol_type = other DNA
ttgcaggctcgtgttgtggggggtggtggtactattgagcaggactactttgggaagctggagagcaagttgagcgttattaggaatctgaacgaccaggttttgtttatcgaccaggggaaccgtcctttgtttgaggacatgacagacagcgacgtcagggacaacgctccaaggacaatcttcatcatctccatgtacaaggactcccagcccaggggaatggccgttacaatcagcgtgaaggtggagaaaatcagcacactgagcgtggagaacaagatcatcagcttcaaggaaatgaacccccccgacaacatcaaggacaccaaatccgacatcatctttttccagaggagtgtgcccggacacgacaacaaaatgcagttcgaatccagctcctacgagggctacttcctggccgtcgaaaaggaaagggacctgttcaagctgatcctgaagaaggaggatgagctgggggacaggtccatcatgttcaccgtgcagaacgaggacgaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 173 - DNA, 1653 bp, synthetic construct
  source 1..1653: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgtcctccctgaagcctgaagatactgctgtgtactactgcgccgccggctatcagatcaattccggaaactacaacttcaaagattacgagtatgactactggggccagggtacccaggtgacagtgagttccgaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgtgatcaattttgtggccatgaaatttattgacaacaccctgtatttcatcgccgaaaacgatgaaaacggcggaggatcttattttggaaagctggaatccaaattgagcgtgatcaggaacctgaacgatcaggtgctgtttatcgaccagggcaacaggcccctgttcgaggatatgaccgacagtgacgtgagggacaacgctccccgaaccatcttcatcatcagtaagtatggagactcccagccaaggggactggccgttacaattagcgtgaaagtcgagaagatttccacattgagcgtggagaacaagatcatctccttcaaggagatgaacccccccgacaacattaaggacaccaaaagcgatatcatcttcttccagaggtccgtgcctgggcacgacaataaagtgcagttcgagtccagctcctacgagggctactttctggcagtggagaaggaaagggacctgttcaagctgatcctgaagaaggaggacgagctgggggacaggtccatcatgttcaccgtccagaatgaggac

SEQ ID NO: 174 - DNA, 1962 bp, synthetic construct
  source 1..1962: mol_type = other DNA
caggttcaactcgtcgagtcaggcggaggagtcgttcaaccaggaagaagcctcagactcgactgcaaagcaagcggcatcaccttcagcaacagcggaatgcactgggtgagacaagcccctggaaaaggcctggagtgggttgctgtgatttggtacgacggcagcaaaagatattacgccgacagtgtgaaaggcagatttacaatctcaagggacaacagcaagaacacactgttcctgcagatgagcagcctgagagccgaggatactgcagtgtattactgcgccaccaacgacgactactggggacaaggaaccctggtgaccgtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggagggtctggtggtggaggatccgggggaggaggttcaggaggcgggggaagtgctgctgaacctgtggaagataattctattaacttcgtggccatgaagttcatcgataatactctgtacttcatcgccgaaaacgatgagaacggaggaggatcttactttggaaaactggaaagcaagctgagcgtgatccggaacctgaacgatcaagtgctgtttatcgaccaaggcaacaggcccctgttcgaggatatgaccgacagcgacagcagggacaacgctcctagaacaatcttcatcatctccaagtacggcgacagccagcccagaggactggctgtgaccataagcgtgaaaagcgagaaaattagcaccctgagcagcgagaacaagatcatatccttcaaagagatgaacccccccgacaacattaaagacaccaaaagcgatatcatcttcttccagagaagcgtgcccggccacgataacaaagtgcaattcgaaagcagcagctacgagggctacttcctggcttccgaaaaggagagggacctgttcaagctgatcctgaagaaggaggacgagctgggggacagatcaataatgttcaccgtgcagaacgaggac

SEQ ID NO: 175 - DNA, 1962 bp, synthetic construct
  source 1..1962: mol_type = other DNA
caggttcaactcgtcgagtcaggcggaggagtcgttcaaccaggaagaagcctcagactcgactgcaaagcaagcggcatcaccttcagcaacagcggaatgcactgggtgagacaagcccctggaaaaggcctggagtgggttgctgtgatttggtacgacggcagcaaaagatattacgccgacagtgtgaaaggcagatttacaatctcaagggacaacagcaagaacacactgttcctgcagatgagcagcctgagagccgaggatactgcagtgtattactgcgccaccaacgacgactactggggacaaggaaccctggtgaccgtgagtagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcggagggtctggtggtggaggatccgggggaggaggttcaggaggcgggggaagtgctgctgaacctgtggaagataatgtgataaacttcgtggccatgaagttcattgacaataccctgtatttcatcgccgagaatgatgaaaacggaggaggatcttacttcggaaaactggaaagcaaactgagcgtgataagaaacctgaacgatcaagtgctgttcatcgaccagggcaaccgccctctgttcgaagatatgaccgacagcgacgtgagggacaacgctcctagaacaatcttcatcatcagcaaatacggcgactcccagcccaggggactggctgtgaccatcagcgtgaaagtggagaagataagcacactgagcgtggagaacaagatcatatcctttaaggagatgaacccccccgacaacatcaaagacaccaaaagcgatatcatcttcttccagagaagcgtgcctgggcacgacaacaaagtgcagttcgagtcaagcagctacgagggctacttcctcgccgttgaaaaggaaagagacttgttcaagctgatcctgaagaaggaggacgagctgggggacagaagcataatgttcaccgttcagaacgaggac

SEQ ID NO: 176 - DNA, 1284 bp, synthetic construct
  source 1..1284: mol_type = other DNA
gagcctaaaagcagcgacaagacccacacctgccccccttgccccgctcctgaagctgctggaggcccttcagtgtttctgtttcctcccaagcccaaggacacactgatgatttccagaacccccgaggtgacatgcgtggtggtggacgtgagccacgaagatcccgaggtgaagtttaattggtacgtggacggcgtggaagtgcacaacgccaagaccaaaccccgcgaggagcagtataatagcacctacagagtggtgagcgtgctgaccgtgctgcatcaggactggctgaacggcaaggaatacaaatgcaaggtgagcaacaaggccctgcctgcccctatcgagaagaccatcagcaaagccaagggacagccaagagaaccccaggtgtacaccctgccccccagcagagaagaaatgaccaagaaccaggtgagcctgtggtgcctggtgaaaggcttctaccccagcgacatcgccgtggagtgggagagcaacggccagccagagaacaactacaagacaacaccccccgtgctggacagcgacggctctttcttcctgtacagtaaactgaccgtggacaaatcaaggtggcagcagggcaacgtgttcagctgctccgtgatgcacgaggccctgcacaaccactatacccagaagagcctgtccctgagccccggcagcggaggaggaggagctgctgaacctgtggaagataattctattaacttcgtggccatgaagttcatcgataatactctgtacttcatcgccgaaaacgatgagaacggaggaggatcttactttggaaaactggaaagcaagctgagcgtgatccggaacctgaacgatcaagtgctgtttatcgaccaaggcaacaggcccctgttcgaggatatgaccgacagcgacagcagggacaacgctcctagaacaatcttcatcatctccaagtacggcgacagccagcccagaggactggctgtgaccataagcgtgaaaagcgagaaaattagcaccctgagcagcgagaacaagatcatatccttcaaagagatgaacccccccgacaacattaaagacaccaaaagcgatatcatcttcttccagagaagcgtgcccggccacgataacaaagtgcaattcgaaagcagcagctacgagggctacttcctggcttccgaaaaggagagggacctgttcaagctgatcctgaagaaggaggacgagctgggggacagatcaataatgttcaccgtgcagaacgaggac

SEQ ID NO: 177 - DNA, 1170 bp, synthetic construct
  source 1..1170: mol_type = other DNA
tcctacttcggcaagctcgaatccaagctctccgtcatccgcaacctcaacgaccaggtcctctttatcgaccagggcaaccgccctctcttcgaagatatgaccgactccgacgtccgcgacaacgctcctaggaccattttcatcatctccatgtacaaggactcccagccccgtggaatggctgttacaatttccgtcaaggtggagaaaatctccaccctgtccgtggagaacaagatcatttctttcaaggagatgaacccccccgacaacatcaaggacaccaagagcgacatcatcttcttccagcgctccgtgcccggacatgataacaaaatgcagttcgagtcctcctcctacgagggctacttcctcgcagtcgaaaaggaaagggacctgttcaagttgatcctgaagaaggaggacgagctcggtgacaggtccatcatgttcaccgtccagaacgaggacgagcctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 178 - DNA, 1182 bp, synthetic construct
  source 1..1182: mol_type = other DNA
ggaggaggaggaagttacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctccatgtacaaggactcccagcccaggggaatggctgttacaatttccgtgaaagtggagaaaatctccaccctgtccgtggaaaacaagatcatcagcttcaaagagatgaacccccccgacaacatcaaagacaccaaatccgacatcatcttcttccagcgctctgtgcccggccatgataacaaaatgcagttcgaaagctcctcctacgaaggctacttcctggccgtcgaaaaggaacgcgacctcttcaagctcatcctgaagaaggaggacgaactgggggacaggagcatcatgttcaccgttcagaacgaagacgagcctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 179 - DNA, 1197 bp, synthetic construct
  source 1..1197: mol_type = other DNA
ggtggaggaggttctggaggtggtggatcatacttcggcaagctggagtccaagctgtccgtgatccggaacctgaacgaccaggtgctgttcatcgaccagggcaacaggcctctcttcgaggatatgaccgactccgacgtgagggataacgctccaaggacaatcttcatcatctccatgtacaaggactcccagcccaggggaatggctgttacaatttccgtgaaagtggagaaaatctccaccctgtccgtggaaaacaagatcatcagcttcaaagagatgaacccccccgacaacatcaaagacaccaaatccgacatcatcttcttccagcgctctgtgcccggccatgataacaaaatgcagttcgaaagctcctcctacgaaggctacttcctggccgtcgaaaaggaacgcgacctcttcaagctcatcctgaagaaggaggacgaactgggggacaggagcatcatgttcaccgttcagaacgaagacgagcctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatactattaatttcgtggccatgaaattcatcgataatactctgtacttcatcgccgaagatgatgaaaatctggaaagtgattactttggaaagctggaaagcaaactgagcgtgatccgcaacctcaacgatcaggtgctgttcatcgaccagggcaataggcccctgttcgaggacatgaccgactctgactccagggacaacgcccctcgaaccattttcatcatctccatgtacaaggactcccagccaaggggcatggccgttacaatttccgtgaagtccgagaaaatctccaccctgagcagcgaaaacaaaatcatctccttcaaagagatgaacccccccgacaacatcaaggacaccaaatccgacatcatcttcttccagaggtccgtgcccggccacgataacaaaatgcagttcgagagcagctcctacgagggctacttcctggcctccgaaaaggaaagggacctgttcaagctgatcctgaagaaggaggacgagctgggggacagaagcatcatgttcaccgtgcagaacgaggac

SEQ ID NO: 181 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatcagattaattttgtggccatgaaattcattgacaataccctgtatttcatcgccgaagatgatgaaaacctggaatctgattactttggaaaactggaaagcaaactgagcgtgatcaggaacctgaacgatcaggtgctgtttatcgaccagggcaacaggcccctctttgaggacatgaccgactctgactctagggacaacgcccccaggaccatcttcatcatctccatgtacaaggactcccagccaaggggaatggccgtgacaatttccgtgaaatccgagaagatctccaccctgtcctccgagaacaaaatcatctccttcaaagagatgaacccccccgacaacatcaaagacacaaaatccgacatcatcttcttccagaggtccgtgcccggtcacgacaacaaaatgcagttcgagagcagcagctacgagggctacttcctcgcctccgaaaaggaaagggacctgttcaagctgatcctgaagaaggaggacgagctgggggacaggtctatcatgttcaccgtccagaacgaggac

SEQ ID NO: 182 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatgatattaatttcgtggccatgaaattcatcgataatactctgtacttcatcgccgaagatgatgaaaacctggaatccgattattttggaaaactggaatccaaactgagcgtgatcaggaacctcaacgaccaggtgctgttcattgaccagggcaacaggcccctgttcgaggatatgacagactccgacgcccgcgacaacgctcctagaacaatcttcatcatctctatgtacaaggactcccagcccaggggaatggccgttacaatttccgtgaaggccgagaagatctccaccctgtccgccgaaaacaagatcatctccttcaaagagatgaacccccccgacaacatcaaggacaccaaatccgacatcatcttcttccagcgctccgtgcccggccatgataacaaaatgcagttcgaaagctcctcctacgagggatactttctggccgccgaaaaggaaagggacctgttcaagctgatcctgaagaaggaggacgagctgggggacagatccatcatgttcaccgttcagaacgaggac

SEQ ID NO: 183 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatttcatcaattttgtggccatgaaattcattgataacaccctgtatttcatcgccgaagatgatgaaaacctggagtctgattactttggaaagctggaaagcaaactgagcgtgatccgcaacctcaacgatcaggtgctgtttatcgaccagggcaacaggccactgttcgaggacatgaccgacagcgacgccagggataacgcccctagaacaatcttcattatctccatgtacaaggactcccagcccaggggaatggcagtgacaatctccgtgaaggccgagaagatcagcaccctgagcgccgaaaacaagatcatctccttcaaggagatgaacccccccgacaacatcaaggacaccaaatccgacatcatcttcttccagaggtccgtgcccggccacgataacaaaatgcagtttgaatcctcctcctacgaaggctactttctggccgccgaaaaggaaagagacttgttcaagctgatcctgaagaaggaggacgagctgggggacagatccatcatgttcaccgttcagaacgaggac

SEQ ID NO: 184 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatctgattaattttgtggccatgaaattcatcgataacaccttgtactttatcgctgaagatgatgaaaatctggaaagtgactacttcggaaaactggaaagcaaattgagcgtgatcaggaatctgaacgatcaggtgctgttcatcgaccagggcaaccgccctctgttcgaggatatgaccgactctgacgtgagggacaacgccccaaggacaatcttcatcatctctatgtacaaggattcccagcccaggggcatggccgttacaatttccgtgaaggtggagaagatctccacactgagcgtggagaacaagatcatctccttcaaggagatgaacccccccgacaacatcaaggacaccaaatccgacatcatctttttccagaggagtgtgcccggtcacgacaacaagatgcagttcgaatccagctcctacgaaggctacttcctcgccgtcgaaaaggaaagggacctgttcaagctgatcctgaagaaggaggacgagttgggggacaggagcatcatgttcaccgtccagaacgaggac

SEQ ID NO: 185 - DNA, 1272 bp, synthetic construct
  source 1..1272: mol_type = other DNA
gaacctaaatcctccgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataatcatattaatttcgtggccatgaaattcatcgataatactctgtacttcatcgccgaagatgatgaaaatctggaaagtgattactttggaaagctggaaagcaaactgagcgtgatcaggaatctcaacgatcaggtgctgttcatcgaccagggcaataggcccctgttcgaggacatgaccgactccgacgtgcgggataacgcccctagaacaatcttcatcatctccatgtacaaggactcccagccaaggggcatggcagtgacaatcagcgtgaaagtggagaagatctccacactgtccgtggagaacaagatcatctccttcaaggagatgaacccccccgacaacatcaaggacacaaagagcgatatcattttcttccagaggagcgtgcccgggcacgataacaaaatgcagttcgaatccagctcctacgaaggctacttcctcgccgtcgaaaaggaaagggacctgttcaagttgatcctgaagaaggaggacgaattgggggacaggtccatcatgttcaccgtccagaacgaggac

SEQ ID NO: 186 - DNA, 642 bp, synthetic construct
  source 1..642: mol_type = other DNA
gacattctgctgacccagagccccgtgatcctgagcgtgagccccggagagagagtgagctttagctgcagagccagccagagcatcggcaccaacatccactggtatcagcagcggaccaacggcagccccagactgctgatcaagtacgccagtgagagcatcagcggaatcccttctaggttcagcggatcaggcagcggcaccgacttcaccctgagtatcaacagcgtggaaagcgaagacatcgccgactattactgccagcagaacaataactggcccaccaccttcggcgccggcaccaagctggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 187 - DNA, 642 bp, synthetic construct
  source 1..642: mol_type = other DNA
gacatccagatgacccagtccccatcctccctgtctgcttctgtgggagacagggtgaccatcacctgcagagcttcccaggacgtgaacacagccgtggcttggtaccagcagaagcctggaaaagcccccaagctgctgatctactccgcctcctttctgtattccggcgtgccttcccggttttccggaagcaggtctggtacagacttcaccctgaccatctcctccctgcagcctgaagattttgctacctactactgccagcagcactacaccacccctcctacattcggccagggtacaaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 188 - DNA, 642 bp, synthetic construct
  source 1..642: mol_type = other DNA
gaaatcgtgctgacccagtcccccgctaccttgagtctgtcccctggagagagagccaccctgtcttgtagggctagtcagtccgtgagcagctatctggcctggtatcagcagaagcccggccaggctcctaggttgttgatttacgatgcctccaacagagccaccggaatccctgcacggttctccggttccggttctggtacagacttcaccctgactatcagcagtctggagcccgaggatttcgccgtttactactgccagcagtcctccaactggcccaggacattcggtcagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 189 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 190 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSRDELTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 191 - protein, 107 aa, synthetic construct
GQPREPQVYTLPPSRDELTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 192 - protein, 107 aa, synthetic construct
GQPRRPRVYTLPPSRDELTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 193 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 194 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDNLTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 195 - protein, 107 aa, synthetic construct
GQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 196 - protein, 107 aa, synthetic construct
GQPREPQVYTLPPSRDNLTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 197 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 198 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 199 - protein, 107 aa, synthetic construct
GQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 200 - protein, 107 aa, synthetic construct
GQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 201 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 202 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 203 - protein, 107 aa, synthetic construct
GQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 204 - protein, 107 aa, synthetic construct
GQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 205 - protein, 107 aa, synthetic construct
GQPREPQVYTLPPSRDELTKNQVSLTCLVKGFYPSDIAVEWESNGQPENNYKTTPPVLDSDGSFFLYSNLTVDKSRWQQGNVFSCSVMHEALHNHYTQKSLSLSPGK

SEQ ID NO: 206 - protein, 107 aa, synthetic construct
GQPREPQVYTLPPSRDELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 207 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSNLTVDKSRWQQGNVFSCSVMHEALHNHYTQKSLSLSPGK

SEQ ID NO: 208 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 209 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 210 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 211 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 212 - protein, 107 aa, synthetic construct
GQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 213 - protein, 107 aa, synthetic construct
GQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 214 - protein, 107 aa, synthetic construct
GQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 215 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVHLTCLVKGFYPSDIAVEWESNGQPENNYKTTPPVLDSDGSFALYSKLTVDKSRWQQGNVFSCSVMHEALHNHYTQKSLSLSPGK

SEQ ID NO: 216 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSRDELTKNQVSLTCLVKGFYPSDIAVEWESNGQPENNYKTFPPVLDSDGSFFLYSKLTVDKSRWQQGNVFSCSVMHEALHNHYTQKSLSLSPGK

SEQ ID NO: 217 - protein, 107 aa, synthetic construct
GQPREPQVYTLPPSRDELTKNQVHLTCLVKGFYPSDIAVEWESNGQPENNYKTTPPVLDSDGSFALYSKLTVDKSRWQQGNVFSCSVMHEALHNHYTQKSLSLSPGK

SEQ ID NO: 218 - protein, 107 aa, synthetic construct
GQPREPQVTTLPPSRDELTKNQVSLTCLVKGFYPSDIAVEWESNGQPENNYKTFPPVLDSDGSFFLYSKLTVDKSRWQQGNVFSCSVMHEALHNHYTQKSLSLSPGK

SEQ ID NO: 219 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 221 - protein, 107 aa, synthetic construct
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 107 aa, synthetic construct
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 223 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 225 - protein, 107 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 227 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCDVSGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 107 aa, synthetic construct
GQPREPQVYTLPPSRDELTKNQVSLTCDVSGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 107 aa, synthetic construct
GQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 232 - protein, 210 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 233 - protein, 107 aa, synthetic construct
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 234 - protein, 107 aa, synthetic construct
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 235 - protein, 4 aa, synthetic construct
GGGS

SEQ ID NO: 236 - protein, 5 aa, synthetic construct
  VARIANT 5: note = X is Q or A or E or S.
GGGGX

SEQ ID NO: 237 - protein, 5 aa, synthetic construct
GGGGS

SEQ ID NO: 238 - protein, 31 aa, synthetic construct
  VARIANT 9: note = X is any amino acid except cysteine; for example X is A or R or N or D or Q or E or G or H or I or L or K or M or F or P or O or S or U or T or W or Y or V.
AAEPVEDNXINFVAMKFIDNTLYFIAEDDEN

SEQ ID NO: 239 - protein, 31 aa, synthetic construct; identical to SEQ ID NO: 238
  VARIANT 9: note = X is A, V, I, L, M, F, Y, or W.

SEQ ID NO: 240 - protein, 31 aa, synthetic construct; identical to SEQ ID NO: 238
  VARIANT 9: note = X is S or T or N or Q.

SEQ ID NO: 241 - protein, 5 aa, synthetic construct
EPKSC

SEQ ID NO: 242 - protein, 5 aa, synthetic construct
EPKSS

SEQ ID NO: 243 - protein, 5 aa, synthetic construct
EPKSA

SEQ ID NO: 244 - protein, 5 aa, synthetic construct
CPPCP

SEQ ID NO: 245 - protein, 8 aa, synthetic construct
APELLGGP

SEQ ID NO: 246 - protein, 8 aa, synthetic construct
APEAAGGP

SEQ ID NO: 247 - protein, 20 aa, synthetic construct
METDTLLLWVLLLWVPGSTG

SEQ ID NO: 248 - protein, 8 aa, synthetic construct
DYKDDDDK

SEQ ID NO: 249 - protein, 6 aa, synthetic construct
  VARIANT 3: note = X is any amino acid
  VARIANT 5: note = X is any amino acid.
IEXDXG

SEQ ID NO: 250 - protein, 193 aa, synthetic construct
MAAEPVEDNCINFVAMKFIDNTLYFIAEDDENIEQDYFGKLESKLSVIRNLNDQVLFIDQGNRPLFEDMTDSDVRDNAPRTIFIISMYKDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 251 - protein, 193 aa, synthetic construct
MAAEPVEDNCINFVAMKFIDNTLYFIAEDDENIEQDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 252 - protein, 14 aa, synthetic construct
KPLGLQARVVGGGG

SEQ ID NO: 253 - protein, 4 aa, synthetic construct
LESD

SEQ ID NO: 254 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 671 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 256 - protein, 359 aa, synthetic construct
EVQLVESGGGLVQAGGSLRLSCAASGRTFSSYAMGWFRQAPGKEREFVVAINWSSGSTYYADSVKGRFTISRDNAKNTMYLQMSSLKPEDTAVYYCAAGYQINSGNYNFKDYEYDYWGQGTQVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNVINFVAMKFIDNTLYFIAEDDENGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 258 - protein, 359 aa, synthetic construct
EVQLVESGGGLVQAGGSLRLSCAASGRTFSSYAMGWFRQAPGKEREFVVAINWSSGSTYYADSVKGRFTISRDNAKNTMYLQMSSLKPEDTAVYYCAAGYQINSGNYNFKDYEYDYWGQGTQVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 259 - protein, 454 aa, synthetic construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 260 - protein, 232 aa, synthetic construct; identical to SEQ ID NO: 19

SEQ ID NO: 261 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMSSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 262 - protein, 424 aa, synthetic construct
AAEPVEDNVINFVAMKFIDNTLYFIAENDENGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 263 - protein, 424 aa, synthetic construct
AAEPVEDNVINFVAMKFIDNTLYFIAENDENGGGSYFGKLKSKLSVIRNLNDQVLFIDQGNRPLFEDMTDSDVRDNAPRTIFIISHYGDSQPRGMAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 264 - protein, 388 aa, synthetic construct
YFGKLQSKLSVIRNLNDQVLFIDQGNRPLFEDMTDSDVRDNAPRTIFIISMYDDSQPRGKAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 265 - protein, 388 aa, synthetic construct
YFGKLQSKLSVIRNLNDQVLKIDQGNRPLFEDMTDSDVRDNAPRTIFIISMYDDSQPRGKAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 266 - protein, 388 aa, synthetic construct
YFGKLQSKLSVIRNLNDQVLFIDQGNRPLFEDMTDSDVRDNAPRTIFIISMYDDSQPRGKAVTISVKVEKISTLSVENKIISFKEMNPPDNIKDTKSDIIFFQRSVPGHDNEMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 267 - protein, 581 aa, synthetic construct
EVQLVESGGGLVQAGGSLRLSCAASGRTFSSYAMGWFRQAPGKEREFVVAINWSSGSTYYADSVKGRFTISRDNAKNTMYLQMSSLKPEDTAVYYCAAGYQINSGNYNFKDYEYDYWGQGTQVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 268 - protein, 394 aa, synthetic construct
GGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 269 - protein, 389 aa, synthetic construct
YFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHRNKVQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 270 - protein, 450 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 393 aa, synthetic construct
GGGGSYFGKLQSKLSVIRNLNDQVLFIDQGNRPLFEDMTDSDVRDNAPRTIFIISMYDDSQPRGKAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 272 - protein, 581 aa, synthetic construct
EVQLVESGGGLVQAGGSLRLSCAASGRTFSSYAMGWFRQAPGKEREFVVAINWSSGSTYYADSVKGRFTISRDNAKNTMYLQMSSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 273 - protein, 671 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSAAEPVEDNVINFVAMKFIDNTLYFIAEDDENIEQDYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNED

SEQ ID NO: 274 - protein, 388 aa, synthetic construct
YFGKLKSKLSVIRNLNDQVLFIDQGNRPLFEDMTDSDVRDNAPRTIFIISMYDDSQPRGRAVTISVKVEKISTLSVENKIISFKEMNPPDNIKDTKSDIIFFQRSVPGHDNKMQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 275 - protein, 551 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAAEPVEDNSINFVAMKFIDNTLYFIAEDDENQDIEYFGKLESKLSVIRNLNDQVLFIDQGNRPLFEDMTDSDSRDNAPRTIFIISKYGDSQPRGLAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 276 - protein, 389 aa, synthetic construct
YFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 429 aa, synthetic construct
AAEPVEDNVINFVAMKFIDNTLYFIAENDENGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 429 aa, synthetic construct; identical to SEQ ID NO: 277

SEQ ID NO: 279 - protein, 450 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 400 aa, synthetic construct
GGGGSYFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHDNKVQFESSSYEGYFLAVEKERDLFKLILKKEDELGDRSIMFTVQNEDGGGGSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 157 aa, synthetic construct
YFGKLQSKLSVIRNLNDQVLFIDQGNRPLFEDMTDSDVRDNAPRTIFIISMYDDSQPRGKAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 284 - protein, 157 aa, synthetic construct
YFGKLQSKLSVIRNLNDQVLKIDQGNRPLFEDMTDSDVRDNAPRTIFIISMYDDSQPRGKAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 285 - protein, 157 aa, synthetic construct
YFGKLQSKLSVIRNLNDQVLFIDQGNRPLFEDMTDSDVRDNAPRTIFIISMYDDSQPRGKAVTISVKVEKISTLSVENKIISFKEMNPPDNIKDTKSDIIFFQRSVPGHDNEMQFESSSYEGYFLAVEKERDLFKLILKKEDELGDRSIMFTVQNED

SEQ ID NO: 286 - protein, 157 aa, synthetic construct
YFGKLKSKLSVIRNLNDQVLFIDQGNRPLFEDMTDSDVRDNAPRTIFIISMYDDSQPRGRAVTISVKVEKISTLSVENKIISFKEMNPPDNIKDTKSDIIFFQRSVPGHDNKMQFESSSYEGYFLAVEKERDLFKLILKKEDELGDRSIMFTVQNED

SEQ ID NO: 287 - protein, 157 aa, synthetic construct
YFGKLESKLSVIRNLNDQVLFIDQGNRPLFEDMTDSDVRDNAPRTIFIISKYGDSQPRGLAVTISVKVEKISTLSVENKIISFKEMNPPDNIKDTKSDIIFFQRSVPGHRNKVQFESSSYEGYFLAVEKERDLFKLILKKEDELGDRSIMFTVQNED

SEQ ID NO: 288 - DNA, 696 bp, synthetic construct; identical to SEQ ID NO: 114
  source 1..696: mol_type = other DNA

SEQ ID NO: 289 - DNA, 1653 bp, synthetic construct
  source 1..1653: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgaacagcctgaagcctgaggacaccgccgtgtactattgcgccgctggatatcagatcaattccggcaactacaacttcaaggactacgagtatgactactggggccagggcacacaggtgacagtgtcatccgaacctaaatcctccgacaaaacccacacctgccccccttgccctgctcctgaattgctgggaggaccctccgtgtttctgtttccccccaaacccaaggacaccctgatgatctccaggacccccgaggtgacctgcgttgttgtggacgtgtcccacgaggaccctgaggttaaatttaactggtacgtcgacggcgtcgaggtccacaacgcaaaaaccaaaccccgcgaggaacagtataacagtacctacagagtggtgtccgttctcaccgtgctgcaccaggattggctgaacggcaaggagtataagtgtaaagtgtccaacaaagccctgcccgcccccattgagaaaactatttctaaagccaaaggccagcccagggaaccacaggtttacacactgcccccctccagagaagaaatgaccaagaaccaggtgagcctgtggtgcctcgtcaaaggattctacccctccgacatcgccgtggagtgggaatcaaatggccagcccgagaacaactacaaaaccaccccccccgtgctcgactccgatggatctttcttcctgtactccaagttgaccgtggacaagtccaggtggcagcagggaaacgtgttcagctgctccgtgatgcacgaggccctgcataatcactatacacagaagagtttgtccctgtctcctggcaaggctgctgaacctgtggaagataatgtgatcaattttgtggccatgaaatttattgacaacaccctgtatttcatcgccgaagatgatgaaaacggcggaggatcttattttggaaagctggaatccaaattgagcgtgatcaggaacctgaacgatcaggtgctgtttatcgaccagggcaacaggcccctctttgaggacatgaccgacagtgacgtgagggacaacgcccctaggaccatcttcatcatctccaagtacggcgattcccagcccaggggactggctgttacaatttccgtgaaggtggagaagatttccaccctgagcgtggagaacaagatcatttccttcaaagagatgaacccccccgacaacatcaaggacaccaaaagcgatatcattttcttccagcgtagcgtgcccggccacgataacaaagtgcagttcgaatcctcctcctacgagggctacttcctggccgtcgaaaaggaaagggacctgttcaagttgatcctgaagaaggaggacgaattgggggacagaagcatcatgttcaccgtgcagaacgaggac

SEQ ID NO: 290 - DNA, 2013 bp, synthetic construct
  source 1..2013: mol_type = other DNA
caggttcagctcaaacagagcggccccggactcgttcagccatctcagtctctctccatcacatgcacagtctccggcttctccctcacaaactacggcgtccactgggtcaggcagtcccctggaaagggactcgaatggctgggagtgatttggtccggcggcaacaccgactacaacacccctttcacctctcggctgtccatcaacaaggacaattccaagtcccaggtctttttcaagatgaatagcctgcagtccaacgacaccgccatctactactgcgcccgggctttgacatactacgactacgagttcgcctactggggccagggaaccttggtgacagtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcgggggatctgggggaggaggtagtggaggcggaggatccggcggtggaggttctggtggtgggggcagtgggggtggcggatcagctgctgaacctgtggaagataatgtgatcaattttgtggccatgaaatttattgacaacaccctgtatttcatcgccgaagatgatgaaaacattgaacaggactatttcggaaagctggaatccaaattgagcgtgatccggaacctgaacgatcaggtgctgttcatcgaccagggcaacaggcctctgttcgaggacatgaccgactccgacgtgagggacaacgcccctagaacaatcttcatcatctccaagtacggagactcccagccaaggggcctggctgttacaatctccgtgaaggtggagaagatctccaccctgtccgtggagaacaagatcatctccttcaaggagatgaacccccccgacaacatcaaggacaccaaatccgacatcatcttcttccagcgctccgtgcctgggcacgataacaaagtccagttcgagtccagctcctacgagggctacttcctggccgtcgaaaaggaaagggacctgttcaagctgatcctgaagaaggaggacgagctgggagacaggagcatcatgttcaccgtccagaacgaggac

SEQ ID NO: 291 - DNA, 1077 bp, synthetic construct
  source 1..1077: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgtcctccctgaagcctgaagatactgctgtgtactactgcgccgccggctatcagatcaattccggaaactacaacttcaaagattacgagtatgactactggggccagggtacccaggtgacagtgagttccgagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 292 - DNA, 1743 bp, synthetic construct
  source 1..1743: mol_type = other DNA
gaagtgcagctggtggagagcggaggaggattggtgcaggcaggaggaagcctgaggctgagttgtgccgctagtggaaggacatttagttcctatgcaatggggtggttcaggcaggccccaggaaaggaaagggagtttgtggtggccatcaactggagtagcggatctacatactacgctgattctgtgaagggcaggttcaccatcagccgggacaatgctaagaacaccatgtacctgcagatgtcctccctgaagcctgaagatactgctgtgtactactgcgccgccggctatcagatcaattccggaaactacaacttcaaagattacgagtatgactactggggccagggtacccaggtgacagtgagttccgagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcgggggatctgggggaggaggtagtggaggcggaggatccggcggtggaggttctggtggtgggggcagtgggggtggcggatcagctgctgaacctgtggaagataatgtgatcaattttgtggccatgaaatttattgacaacaccctgtatttcatcgccgaagatgatgaaaacattgaacaggactatttcggaaagctggaatccaaattgagcgtgatccggaacctgaacgatcaggtgctgttcatcgaccagggcaacaggcctctgttcgaggacatgaccgactccgacgtgagggacaacgcccctagaacaatcttcatcatctccaagtacggagactcccagccaaggggcctggctgttacaatctccgtgaaggtggagaagatctccaccctgtccgtggagaacaagatcatctccttcaaggagatgaacccccccgacaacatcaaggacaccaaatccgacatcatcttcttccagcgctccgtgcctgggcacgataacaaagtccagttcgagtccagctcctacgagggctacttcctggccgtcgaaaaggaaagggacctgttcaagctgatcctgaagaaggaggacgagctgggagacaggagcatcatgttcaccgtccagaacgaggac

SEQ ID NO: 293 - DNA, 1077 bp, synthetic construct
  source 1..1077: mol_type = other DNA
gaagtgcagctggtggagagcggcggcggactggtccaggctggaggcagcctgcggctgtcctgtgccgcttctggcaggaccttctccagctatgccatgggatggtttcggcaggcccctggaaaagagcgcgagttcgtggtggccatcaactggagtagcggttctacatattacgcagatagcgtcaagggtcgttttactatttcccgagataacgccaagaacaccatgtacctgcagatgtcatctctgaagccagaggatactgctgtgtactattgtgccgccggctatcagatcaactccggcaactacaactttaaggattacgagtacgattactggggacagggtacacaggtcaccgtgtcaagtgagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 294 - DNA, 1362 bp, synthetic construct
  source 1..1362: mol_type = other DNA
gagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccggttcacgcagaagagcctctccctgtctccgggtaaaggaggcgggggatctgggggaggaggtagtggaggcggaggatccggcggtggaggttctggtggtgggggcagtgggggtggcggatcagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 295 - DNA, 696 bp, synthetic construct; identical to SEQ ID NO: 132
  source 1..696: mol_type = other DNA

SEQ ID NO: 296 - DNA, 1344 bp, synthetic construct
  source 1..1344: mol_type = other DNA
gaagtgcagctggtggagagcggaggaggattggtgcagcctggaggaagcctgaggctgagttgtgccgctagtgggtttaccttttccgactcctggattcattgggtgaggcaggcccccggaaagggattggaatgggtggcatggatttccccctatgggggaagcacctactacgctgattccgtgaagggcaggtttacaatctccgccgacacctccaagaacaccgcctacctgcagatgagctccctgagggctgaggacaccgctgtttactactgcgcccgccgacattggcccggaggattcgattactggggccagggaaccctcgtgaccgtttcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaacagattcacgcagaagagcctctccctgtctccgggtaaa

SEQ ID NO: 297 - DNA, 1272 bp, synthetic construct
  source 1..1272: mol_type = other DNA
gctgctgaacctgtggaagataatgtgatcaatttcgtggccatgaaattcatcgataacaccctgtacttcatcgctgaaaatgatgaaaacggaggaggatcttactttggaaagctggaatccaaactgagcgtgatcaggaacctcaacgaccaggtgctgttcatcgaccagggcaacaggccactcttcgaggatatgacagacagcgacgtgagggacaatgcacccagaaccatcttcatcatctccaagtacggcgacagccagcccagaggactggctgtgacaatctccgtgaaagtggagaagatttccacactgagcgtggagaacaagatcatctccttcaaggagatgaacccccccgacaacattaaagacaccaaatccgacatcatcttcttccagaggtccgtgcccggccatgataacaaagtgcagttcgagtccagctcctacgagggatactttctggccgtcgaaaaggagagggacctgttcaagctcattctcaagaaggaggacgagctgggggacagatccattatgttcaccgtgcagaatgaggacgaacctaagagctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgagcctgagccccggcaag

SEQ ID NO: 298 - DNA, 1272 bp, synthetic construct
  source 1..1272: mol_type = other DNA
gctgctgaacctgtggaagataatgtgatcaatttcgtggccatgaagttcattgacaacaccctgtacttcatcgctgaaaacgatgagaacggcggaggatcttacttcggaaaactgaaatccaagctgagcgtgatcaggaacctgaacgatcaggtgctgttcatcgaccagggcaataggcccctgttcgaggatatgacagactccgacgtgagggacaacgctccaagaaccatcttcatcatctctcactacggcgactcccagccaaggggaatggctgtgacaatctccgtgaaggtggagaagatttccaccctgtccgttgagaacaagatcatctccttcaaagagatgaacccccccgacaacatcaaagacaccaaatccgacatcatcttcttccagcgcagcgtgcctgggcatgataacaaaatgcagttcgaaagctcctcctacgagggatacttcctcgccgtcgaaaaggaaagggacctgttcaagctgatcctgaagaaggaggatgaattgggggataggagcatcatgttcaccgtccagaacgaagacgagcctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacaggttcacccagaagagcctgagcctgagccccggcaag

SEQ ID NO: 299 - DNA, 1164 bp, synthetic construct
  source 1..1164: mol_type = other DNA
tacttcggcaagctgcagtccaagctgtccgtgatcaggaatctcaacgaccaggtgttgttcatcgaccaggggaacaggcccctcttcgaggatatgaccgactccgacgtgagggataacgctccccgtacaatcttcatcatctccatgtacgacgactcccagcccaggggtaaggctgttaccattagcgtgaaagtggagaaaatttccacactgagcgtggagaacaagatcatctcctttaaggagatgaacccccccgacaacattaaagacacaaagagcgacatcatctttttccagaggagtgtgcccggccacgacaacaagatgcagttcgagtccagctcctacgaaggatacttcctggccgtcgaaaaggaaagggacctgttcaagttgatcctgaagaaggaggatgaattgggggacaggagcatcatgttcaccgtccagaacgaggacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgtccctgtcccccgga

SEQ ID NO: 300 - DNA, 1164 bp, synthetic construct
  source 1..1164: mol_type = other DNA
tacttcggcaagctgcagtccaagctgtccgtgatcaggaatctcaacgaccaggtgttgaagatcgaccaggggaacaggcccctcttcgaggatatgaccgactccgacgtgagggataacgctccccgtacaatcttcattatctccatgtacgacgactcccagcccaggggtaaagccgttacaatttccgtgaaggtggagaagatttccacattgtccgtggagaacaagatcatctccttcaaagagatgaacccccccgacaacatcaaggacaccaaaagcgacatcatcttcttccagcgcagtgtgcctgggcacgacaacaaaatgcagttcgaatccagctcctacgagggctactttctggccgtcgaaaaggaaagggacctgttcaaactgatcctgaagaaggaggacgagctgggcgacaggtccatcatgttcaccgtccagaacgaggacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgtccctgtcccccgga

SEQ ID NO: 301 - DNA, 1164 bp, synthetic construct
  source 1..1164: mol_type = other DNA
tacttcggcaagctgcagtccaagctgtccgtgatcaggaatctcaacgaccaggtgttgttcatcgaccaggggaacaggcccctcttcgaggatatgaccgactccgacgtgagggataacgctccccgtacaatcttcatcatctccatgtacgacgactcccagcccaggggtaaggctgttaccattagcgtgaaagtggagaaaatttccacactgagcgtggagaacaagatcatctcctttaaggagatgaacccccccgacaacattaaagacacaaagagcgacatcatctttttccagaggagtgtgcccggccacgacaacgaaatgcagttcgagtccagctcctacgaaggatacttcctggccgtcgaaaaggaaagggacctcttcaagctgatcctgaagaaggaggacgagttgggggacagaagcatcatgtttaccgtgcagaacgaggacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgtccctgtcccccgga

SEQ ID NO: 302 - DNA, 1164 bp, synthetic construct
  source 1..1164: mol_type = other DNA
tacttcggcaagctgaagtccaagctgtccgtgatcaggaatctgaacgaccaggtgctgtttatcgaccagggcaacaggcccctgttcgaggatatgaccgactccgacgtgagggacaacgcaccaaggaccatcttcatcatctccatgtacgacgactcccagccaaggggcagggctgttacaatctccgtgaaggtggagaagatttccacactgagcgtggaaaacaagatcatctccttcaaagagatgaacccccccgacaacatcaaggacaccaaatccgacatcatcttcttccagcgcagcgtgcccggccatgataacaagatgcagttcgaaagctcctcctacgagggatactttctggccgtcgaaaaggaaagggacctgttcaagctgatcctgaagaaggaggacgagttgggggacagaagcatcatgttcaccgtccagaacgaagacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgtccctgtcccccgga

SEQ ID NO: 303 - DNA, 1182 bp, synthetic construct
  source 1..1182: mol_type = other DNA
ggaggaggaggaagttactttggaaagctggagagcaagctgagtgtgatcaggaacctgaatgaccaggtgttgtttattgatcaggggaataggcccttgtttgaggatatgaccgacagcgatgtgagggacaacgccccaaggacaatcttcatcatctccaagtacggcgatagccagcccaggggtttggcagttaccatcagcgtgaaggtggagaagattagcacactgagcgtggagaacaagatcatctccttcaaggagatgaacccccctgacaacatcaaggacaccaagagcgatattatctttttccagaggagtgtgcccggccacgacaacaaagtgcagttcgaaagctcctcctacgagggctacttcctggccgtcgagaaggagagggacctgttcaagctgatcctgaagaaggaggacgagttgggggacaggagcatcatgttcaccgtccagaacgaagacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgagcctgagccccggcaag

SEQ ID NO: 304 - DNA, 1167 bp, synthetic construct
  source 1..1167: mol_type = other DNA
tactttggaaagctggagagcaagctgagtgtgatcaggaacctgaatgaccaggtgttgtttattgatcaggggaataggcccttgtttgaggatatgaccgacagcgatgtgagggacaacgccccaaggacaatcttcatcatctccaagtacggcgatagccagcccaggggtttggcagttaccatcagcgtgaaggtggagaagattagcacactgagcgtggagaacaagatcatctccttcaaggagatgaacccccctgacaacatcaaggacaccaagagcgatattatctttttccagaggagtgtgcccggccaccgtaacaaagtgcagttcgaaagctcctcctacgagggctacttcctggccgtcgagaaggagagggacctgttcaagctgatcctgaagaaggaggacgagttgggggacaggagcatcatgttcaccgtccagaacgaagacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgagcctgagccccggcaag

SEQ ID NO: 305 - DNA, 1350 bp, synthetic construct
  source 1..1350: mol_type = other DNA
gaagtgcagctgcaggagtccggtcctggattggtgaagcccagccagagcctgtccctgacatgctctgtgacaggatactccatcacaagctcctacaggtggaactggattaggaagttccccggcaacaggctggagtggatgggatacatcaacagcgccggaatctccaactacaacccctccctgaaaaggcgcatcagcatcactagggacacctccaagaaccagttcttcctccaggtgaacagcgtgaccaccgaggacgcagcaacatactactgcgccaggtccgacaacatgggcacaacccctttcacatactggggacagggcaccctcgtgaccgtttcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 306 - DNA, 1179 bp, synthetic construct
  source 1..1179: mol_type = other DNA
ggaggaggaggaagttacttcggcaagctgcagtccaagctgtccgtgatcaggaatctcaacgaccaggtgttgttcatcgaccaggggaacaggcccctcttcgaggatatgaccgactccgacgtgagggataacgctccccgtacaatcttcatcatctccatgtacgacgactcccagcccaggggtaaggctgttaccattagcgtgaaagtggagaaaatttccacactgagcgtggagaacaagatcatctcctttaaggagatgaacccccccgacaacattaaagacacaaagagcgacatcatctttttccagaggagtgtgcccggccacgacaacaagatgcagttcgagtccagctcctacgaaggatacttcctggccgtcgaaaaggaaagggacctgttcaagttgatcctgaagaaggaggatgaattgggggacaggagcatcatgttcaccgtccagaacgaggacgaacctaaatcctccgacaagacacacacctgccccccctgccccgcacctgaggctgctggaggacctagtgtgttcctgttcccccccaaacccaaggacaccctgatgattagcagaacccccgaggtgacctgcgtggtcgtggacgtctcccacgaagaccccgaagtgaaatttaactggtacgtggacggcgtcgaggtgcacaacgcaaagaccaagcccagagaagagcaatataatagcacctatagagtggtgagcgtgctcaccgtgctgcaccaggactggctgaacgggaaggagtataagtgcaaggtgagcaacaaggcactgcctgcccccatagaaaaaaccatcagcaaggccaagggccagcccagagagccccaggtgtacaccctgccccccagcagagaggagatgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaacagattcacccagaagagcctgtccctgtcccccgga

SEQ ID NO: 307 - DNA, 1743 bp, synthetic construct
  source 1..1743: mol_type = other DNA
gaagtgcagctggtggagagcggcggcggactggtccaggctggaggcagcctgcggctgtcctgtgccgcttctggcaggaccttctccagctatgccatgggatggtttcggcaggcccctggaaaagagcgcgagttcgtggtggccatcaactggagtagcggttctacatattacgcagatagcgtcaagggtcgttttactatttcccgagataacgccaagaacaccatgtacctgcagatgtcatctctgaagccagaggatactgctgtgtactattgtgccgccggctatcagatcaactccggcaactacaactttaaggattacgagtacgattactggggacagggtacacaggtcaccgtgtcaagtgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcgggggatctgggggaggaggtagtggaggcggaggatccggcggtggaggttctggtggtgggggcagtgggggtggcggatcagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 308 - DNA, 2013 bp, synthetic construct
  source 1..2013: mol_type = other DNA
caggtgcagctgaagcagtctggaccaggactcgtgcagccaagtcagtctctctccatcacatgcacagtctccggcttctccctcacaaactacggcgtccactgggtgcgacagtcacctggaaaaggcctcgaatggctcggcgtgatctggtccggaggaaataccgactacaacaccccctttacctccaggctgtccatcaacaaggataattccaagtcccaggtgttcttcaagatgaatagcctgcagtccaacgataccgccatctactactgcgccagggctctgacatactacgactacgagttcgcctactggggccagggaacactggtgaccgtgtccgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcgggggatctgggggaggaggtagtggaggcggaggatccggcggtggaggttctggtggtgggggcagtgggggtggcggatcagctgctgaacctgtggaagataatgtgatcaatttcgtggccatgaagttcattgacaacaccctgtacttcatcgctgaagatgatgaaaacatcgagcaggactatttcggaaaactggaaagcaaactgagcgtgatcaggaacctgaacgaccaggtgctgtttatcgaccagggcaacaggcccctgttcgaagatatgaccgacagcgacgtcagggacaacgctcctagaacaatcttcatcatctccaagtacggcgactcccagccaaggggactggctgtgaccatttccgtgaaagtggagaagatctccacactgagcgtggaaaacaaaatcatctccttcaaggagatgaacccccccgacaacattaaagacaccaaaagcgatatcatcttcttccagaggagtgtgcccggccacgataacaaagtgcagttcgagagcagctcctacgagggatactttctggccgtcgaaaaggagagggacctgttcaagctgatcctgaagaaggaggacgagctgggggacagatcaatcatgttcaccgtgcagaacgaagac

SEQ ID NO: 309 - DNA, 1653 bp, synthetic construct
  source 1..1653: mol_type = other DNA
gaagtgcagctggtggaaagcggaggaggattggtgcaggcaggaggaagtctgaggctgagttgtgccgctagcggtagaacatttagtagttatgctatggggtggtttaggcaggctcctggaaaggagagggagttcgtggtggctatcaactggagcagcggctctacatactacgctgattccgtgaagggaaggttcacaatctccagggacaatgctaagaacacaatgtatctgcagatgaacagcctcaagcctgaggacaccgccgtttactattgcgccgctggataccaaatcaacagcggcaactacaacttcaaggactatgagtatgactactggggccagggcacacaggtgacagtttcatccgagcccaaatcttctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgctgaacctgtggaagataattctattaatttcgtggccatgaaattcatcgataatactctgtacttcatcgccgaagatgatgaaaatcaggatattgaatacttcggaaaactggaatccaaattgagcgtgatccggaacctgaacgatcaggtgctgttcatcgaccagggcaatcggccactcttcgaggacatgaccgactcagactccagggacaacgctccccgaaccatcttcatcatctccaagtacggcgactcccagcccaggggattggctgttaccatttctgtgaagtctgagaagatctccaccctgtcctccgagaacaaaatcatctccttcaaggagatgaacccccccgacaacatcaaggacaccaagagcgacatcatttttttccagcgctccgtgcccggtcacgacaataaggtccagttcgaatcctccagctacgagggctactttctggcctccgaaaaggaaagagacttgttcaagctgattctgaaaaaggaggacgagctgggcgacaggtccatcatgttcaccgtgcagaacgaggac

SEQ ID NO: 310 - DNA, 1947 bp, synthetic construct
  source 1..1947: mol_type = other DNA
gaagtgcagctggtggagagcggaggaggattggtgcagcctggaggaagcctgaggctgagttgtgccgctagtgggtttaccttttccgactcctggattcattgggtgaggcaggcccccggaaagggattggaatgggtggcatggatttccccctatgggggaagcacctactacgctgattccgtgaagggcaggtttacaatctccgccgacacctccaagaacaccgcctacctgcagatgagctccctgagggctgaggacaccgctgtttactactgcgcccgccgacattggcccggaggattcgattactggggccagggaaccctcgtgaccgtttcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggaggaggttctggaggtggtggatcagctgctgaacctgtggaagataatgtgatcaattttgtggccatgaaatttattgacaacaccctgtatttcatcgccgaaaacgatgaaaacggcggaggatcttattttggaaagctggaatccaaattgagcgtgatcaggaacctgaacgatcaggtgctgtttatcgaccagggcaacaggcccctgttcgaggatatgaccgacagtgacgtgagggacaacgctccccgaaccatcttcatcatcagtaagtatggagactcccagccaaggggactggccgttacaattagcgtgaaagtcgagaagatttccacattgagcgtggagaacaagatcatctccttcaaggagatgaacccccccgacaacattaaggacaccaaaagcgatatcatcttcttccagaggtccgtgcctgggcacgacaataaagtgcagttcgagtccagctcctacgagggctactttctggcagtggagaaggaaagggacctgttcaagctgatcctgaagaaggaggacgagctgggggacaggtccatcatgttcaccgtccagaatgaggac

SEQ ID NO: 311 - DNA, 642 bp, synthetic construct
  source 1..642: mol_type = other DNA
gatatccagatgacccagtccccctcctccctgtctgcttctgtgggagacagagtgaccattacctgtagagcctcccaggacgtgtccaccgctgttgcttggtaccagcagaagcccggcaaggctccaaaactgctcatctactccgcctcctttctgtactccggcgtgccatcccgcttctcaggatctggatctggaaccgacttcaccttgaccatctcctccctgcagcccgaggacttcgctacttactactgccagcagtacctctaccaccccgctaccttcggccagggaacaaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 312 - DNA, 654 bp, synthetic construct
  source 1..654: mol_type = other DNA
gacatcgtgatgacccagggaaccctcccaaacccagtgccttctggagaatccgtgagcattacctgcaggtcctccaaatccctgctgtactccgacggaaaaacctacctgaactggtacctgcagaggcccggccagtccccccagctgctgatctactggatgtccaccagggcctccggcgtgtccgacaggttctccggctccggctccggcaccgacttcaccctgaagatctccggcgtggaggccgaggacgtgggcatctactactgccagcagggcctggagttccccaccttcggcggcggcaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 313 - DNA, 1200 bp, synthetic construct
  source 1..1200: mol_type = other DNA
ggaggaggaggaagttactttggaaagctggagagcaagctgagtgtgatcaggaacctgaatgaccaggtgttgtttattgatcaggggaataggcccttgtttgaggatatgaccgacagcgatgtgagggacaacgccccaaggacaatcttcatcatctccaagtacggcgatagccagcccaggggtttggcagttaccatcagcgtgaaggtggagaagattagcacactgagcgtggagaacaagatcatctccttcaaggagatgaacccccctgacaacatcaaggacaccaagagcgatattatctttttccagaggagtgtgcccggccacgacaacaaagtgcagttcgaaagctcctcctacgagggctacttcctggccgtcgagaaggagagggacctgttcaagctgatcctgaagaaggaggacgagttgggggacaggagcatcatgttcaccgtccagaacgaagacggtggcggagggtctggtggtggaggatccgggggaggaggttcaggaggcgggggaagtgacatcgtgatgacccagggaaccctcccaaacccagtgccttctggagaatccgtgagcattacctgcaggtcctccaaatccctgctgtactccgacggaaaaacctacctgaactggtacctgcagaggcccggccagtccccccagctgctgatctactggatgtccaccagggcctccggcgtgtccgacaggttctccggctccggctccggcaccgacttcaccctgaagatctccggcgtggaggccgaggacgtgggcatctactactgccagcagggcctggagttccccaccttcggcggcggcaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 314 - DNA, 1167 bp, synthetic construct
  source 1..1167: mol_type = other DNA
tacttcggcaagctggagtccaagctgtccgtgatcaggaatctcaacgaccaggtgctgttcatcgaccaggggaacaggcccctcttcgaggatatgaccgactccgacgtgagggataacgctcccaggacaatcttcatcatttccaagtacggggattcccagccaaggggactggctgttacaatttccgtgaaggtggagaagatcagcacactctccgtggagaacaagatcatcagcttcaaagagatgaacccccccgacaacattaaggacaccaaaagcgatatcatcttcttccagcgctccgtgcctggccacgataacaaggtgcagttcgagagctcctcctacgaaggctactttctggccgtcgaaaaggagagggacctgttcaagttgatcctgaagaaggaggacgagctgggcgacaggagcatcatgttcaccgtccagaatgaggacgaacctaaatcctccgacaagacccacacctgccccccttgccccgctcctgaagctgctggaggcccttcagtgtttctgtttcctcccaagcccaaggacacactgatgatttccagaacccccgaggtgacatgcgtggtggtggacgtgagccacgaagatcccgaggtgaagtttaattggtacgtggacggcgtggaagtgcacaacgccaagaccaaaccccgcgaggagcagtataatagcacctacagagtggtgagcgtgctgaccgtgctgcatcaggactggctgaacggcaaggaatacaaatgcaaggtgagcaacaaggccctgcctgcccctatcgagaagaccatcagcaaagccaagggacagccaagagaaccccaggtgtacaccctgccccccagcagagaagaaatgaccaagaaccaggtgagcctgtggtgcctggtgaaaggcttctaccccagcgacatcgccgtggagtgggagagcaacggccagccagagaacaactacaagacaacaccccccgtgctggacagcgacggctctttcttcctgtacagtaaactgaccgtggacaaatcaaggtggcagcagggcaacgtgttcagctgctccgtgatgcacgaggccctgcacaaccactatacccagaagagcctgtccctgagccccggcaag

SEQ ID NO: 315 - DNA, 1287 bp, synthetic construct
  source 1..1287: mol_type = other DNA
gctgctgaacctgtggaagataatgtgatcaattttgtggccatgaaatttattgacaacaccctgtatttcatcgccgaaaacgatgaaaacggcggaggatcttattttggaaagctggaatccaaattgagcgtgatcaggaacctgaacgatcaggtgctgtttatcgaccagggcaacaggcccctgttcgaggatatgaccgacagtgacgtgagggacaacgctccccgaaccatcttcatcatcagtaagtatggagactcccagccaaggggactggccgttacaattagcgtgaaagtcgagaagatttccacattgagcgtggagaacaagatcatctccttcaaggagatgaacccccccgacaacattaaggacaccaaaagcgatatcatcttcttccagaggtccgtgcctgggcacgacaataaagtgcagttcgagtccagctcctacgagggctactttctggcagtggagaaggaaagggacctgttcaagctgatcctgaagaaggaggacgagctgggggacaggtccatcatgttcaccgtccagaatgaggacggtggaggaggttctggaggtggtggatcagacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 316 - DNA, 1350 bp, synthetic construct
  source 1..1350: mol_type = other DNA
gaggttcagttgcaggagagcggaccaggactggttaagccaagccagagcctgagcttgacatgtagcgtgacaggatacagcattacaagcagctataggtggaactggattaggaagttccccgggaacaggttggagtggatgggatatatcaacagcgcaggaatttccaactacaaccccagcctcaagaggaggatcagcatcacccgggacacctcaaagaaccagttcttcctgcaggtcaacagcgtgaccaccgaggatgctgcaacctactactgcgcccgatccgataacatgggcaccacaccattcacctactgggggcagggaaccttggtgacagtctcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 317 - protein, 5 aa, synthetic construct
  VARIANT 1: note = X is Q, A, E or S
XGGGG

SEQ ID NO: 318 - protein, 5 aa, synthetic construct
  VARIANT 2: note = X is Q, A, E or S
GXGGG

SEQ ID NO: 319 - protein, 5 aa, synthetic construct
  VARIANT 3: note = X is Q, A, E or S
GGXGG

SEQ ID NO: 320 - protein, 5 aa, synthetic construct
  VARIANT 4: note = X is Q, A, E or S
GGGXG

(19) United States
(12) Patent Application Publication
(10) Pub. No.: US 2026/0274968 A1
(43) Pub. Date: Sep. 17, 2026

(54) METHODS OF USING IL-18 FUSION PROTEINS

(71) Applicant: FUSE BIOTHERAPEUTICS INC., Wolburn, MA (US)

(72) Inventors: Brian RABINOVICH, Winchester, MA (US); Jeffrey TAKIMOTO, Cerritos, CA (US); Xueyuan ZHOU, Belmont, MA (US)

(21) Appl. No.: 19/480,303

(22) PCT Filed: May 1, 2024
(86) PCT No.: PCT/US2024/027248

§ 371 Date: Oct. 30, 2025

Related U.S. Application Data
(60) Provisional application No. 63/463,507, filed on May 2, 2023.
(60) Provisional application No. 63/530,780, filed on Aug. 4, 2023.
(60) Provisional application No. 63/573,711, filed on Apr. 3, 2024.

(51) Int. Cl. C07K 16/28; A61K 38/00; A61P 35/00; C07K 14/54; C07K 14/715

(52) U.S. Cl. CPC C07K 16/2863; A61P 35/00; C07K 14/54; C07K 14/7155; A61K 38/00; C07K 2317/569; C07K 2317/732; C07K 2319/01; C07K 2319/30; C07K 2319/50; C07K 2319/70

(57) ABSTRACT

Described herein are method of using fusion proteins composed of elements such as one or more proteins/polypeptides capable of translocating into an endoplasmic reticulum (ER) and an interleukin 18 (IL-18) or a fragment or variant of IL-18. The proteins/polypeptides capable of translocating into the ER include but are not limited to an immunoglobular protein such as Fc region, VHH antibody, and scFv, and a globular protein such as serum albumin. The IL-18 may be a precursor IL-18 that resembles the natural pro-cytokine in which IL-18 is fused to the C-terminus of its propeptide or a propeptide variant. These fusion proteins can be used in methods of treating cancer or fibrosis by activating an IL-18 receptor complex (RC) signal on a cell, releasing or exposing mature IL-18 in a tumor microenviromnent or adjacent to a tumor and/or increasing memory CD8⁺ T cell proliferation.

### CROSS-REFERENCE TO RELATED APPLICATIONS

This application includes a claim of priority under 35 U.S.C. § 119(e) to U.S. provisional patent application No. 63/463,507, filed May 2, 2023, U.S. provisional patent application No. 63/530,780, filed Aug. 4, 2023, and U.S. provisional patent application No. 63/573,711, filed Apr. 3, 2024, the entirety of all which is hereby incorporated by reference.

### REFERENCE TO SEQUENCE LISTING

This application contains a Sequence Listing submitted as a computer readable form named “096034_000004WOPT_SequenceListing.xml”, having a size in bytes of 476,891 bytes, and created on Apr. 30, 2024. The information contained in this computer readable form is hereby incorporated by reference in its entirety.

### FIELD OF INVENTION

This invention relates to methods of using recombinantly produced IL-18 cytokine and fusion proteins thereof, for example, in the treatment of cancer and fibrosis.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

IL-18 is a danger induced cytokine that serves to promote the activity and survival of antigen presenting cells, NK cells and subsets of T cells. The cytokine is strongly regulated by a negative feedback loop that includes the induction of IL-18BP and IL-37, which act to bind IL-18 and IL-18Rα, respectively. In the tumor microenvironment (TME), the IL-18Rα/β complex is expressed on PD-1⁺ progenitor exhausted T cells that are responsive to PD-1 antagonism.

There is a need in the art for targeting an IL-18BP resistant variant of IL-18, which is attenuated for safety, but retains activity when brought into close proximity of the IL-18 receptor complex to maintain survival of the αPD-1 responsive T cell pool. Additionally, many tumors, such as pancreatic cancer, are highly fibrotic. It is very difficult for lymphocytes to enter these tumors. Therefore, there is a need in the art for a therapeutic that can suspend or reduce tumor fibrosis can aid in the infiltration of lymphocytes into the tumor microenvironment.

Furthermore, there remains a need in the art for methods of treating cancer or fibrosis.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of activating an IL-18 receptor complex (RC) signal on a cell, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER.

Various embodiments of the invention provide for a method of releasing or exposing mature IL-18 in a tumor microenvironment or adjacent to a tumor, comprising: administering a fusion protein to a subject in need thereof, wherein the fusion protein comprises a first polypeptide or protein capable of translocating into an endoplasmic reticulum (ER) or a fragment thereof and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER.

Various embodiments of the invention provide for a method of increasing activity, persistence and immune memory of CD8⁺ T cells. CD4⁺ T cells, or γδ T cells, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER.

Various embodiments of the invention provide for a method of treating cancer in a subject in need thereof, comprising: administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant or the fragment of the IL-18 variant is on the C-terminus end of the fusion protein relative to the first polypeptide or protein capable of translocating into the ER. In various embodiments, the subject may have or may be suspected to have cancer, or the subject has one or more symptoms of cancer.

Various embodiments of the invention provide for a method of reducing collagen deposition into extracellular space or reducing fibrosis, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant. In various embodiments, the subject may have or may be suspected to have a fibrotic tissue environment.

In various embodiments, the fusion protein may further comprise a targeting polypeptide. In various embodiments, the targeting polypeptide targets a protein on a cell surface, wherein the cell surface also has an IL-18 RC or the cell is capable of expressing the IL-18 RC. In various embodiments, the targeting polypeptide targets a protein on a cell surface that does not have an IL-18 RC or the cell is not capable of expressing the IL-18 RC.

In various embodiments, the fusion protein can bind to a cell having an IL-18 RC or capable of expressing the IL-18 RC upon activation of the cell, and activates the IL-18 RC signal.

In various embodiments, the targeting polypeptide can comprise a tumor associated antigen binding domain.

In various embodiments, the fusion protein can further comprise an antibody or antibody fragment, and the fusion protein binds to a tumor cell, or to an immune cell or stromal cell in a tumor tissue, or to a tumor draining lymph node, or other secondary lymphoid organ. In various embodiments, antibody fragment can be an Fc fragment.

In various embodiments, the fusion protein can further comprise a masking domain. In various embodiments, a mature IL-18 or mature IL-18 variant can be released from a masking domain by a protease. In various embodiments, the protease can be granzyme or a metalloprotease. In various embodiments, the granzyme can be released from an immune cell. In various embodiments, the immune cell can be an NK cell, a T cell, a neutrophil, or a mast cell. In various embodiments, the antibody or antibody fragment can recruit the immune cell to the tumor cell. In various embodiments, the metalloprotease can be expressed in a tumor microenvironment or tumor draining lymph node.

In various embodiments, the fusion protein can further comprise half-life extending molecule. In various embodiments, the half-life extending molecule can be a half-life extending polypeptide. In various embodiments, the half-life extending polypeptide can be a human serum albumin (HSA) or an HSA-binding fragment.

In various embodiments, the fusion protein can have reduced activity as compared to wild-type IL-18 when not bound to a cell having the IL-18 RC. In various embodiments, the reduced activity can be at least a 75% reduction in activity as compared to wild-type IL-18.

In various embodiments, the mature IL-18 can increase the activity of NK cells or T cells, and optionally the activity being one or more of proliferation, survival, and cytotoxicity.

In various embodiments, the method can further comprise administering an immunotherapeutic agent. In various embodiments, the immunotherapeutic agent can comprise an immune check point inhibitor.

In various embodiments, the fusion protein, the IL-18, the fragment of IL-18, the IL-18 variant, or the fragment of the IL-18 variant can be fused to the N-terminus of a knob of a knob-into-hole heterodimeric IgG1 protein with or without a propeptide (PP) or a PP variant.

In various embodiments, the fusion protein, the IL-18, the fragment of IL-18, the IL-18 variant, or the fragment of the IL-18 variant can be fused to the C-terminus of a knob of a knob-into-hole heterodimeric IgG1 protein with or without a propeptide (PP) or a PP variant.

In various embodiments, the fusion protein, the IL-18, a fragment of IL-18, an IL-18 variant, or a fragment of the IL-18 variant can further comprise its propeptide (PP) or a PP variant.

In various embodiments, the IL-18 propeptide variant can comprise a polypeptide having AAEPVEDNX₁INFVAMKFIDNTLYFIAEDDEN (SEQ ID NO:238), wherein X₁ can be any amino acid except cysteine. In various embodiments, X₁ can be alanine, valine, isoleucine, leucin, methionine, phenylalanine, tyrosine or tryptophan. In various embodiments, X₁ can be valine. In various embodiments. X₁ can be serine, threonine, asparagine, or glutamine. In various embodiments, X₁ can be serine.

In various embodiments, the fusion protein does not comprise a polypeptide consisting of the sequence X₁-X₂-X₃-X₄ between the propeptide or propeptide variant, and the mature IL-18 or mature IL-18 variant, wherein X₁ can be L or absent, X₂ can be E or absent, X₃ can be S or absent, and X₄ can be D or absent.

In various embodiments, the PP or the PP variant can be on the N-terminus end relative to the IL-18, the fragment of IL-18, the IL-18 variant, or the fragment of the IL-18 variant. In various embodiments, the propeptide or propeptide variant can serve as a masking domain.

In various embodiments, the fusion protein can further comprise one or more cleavage sites and the fusion protein can be cleaved at the one or more cleavage sites by one or more proteases. In various embodiments, the one or more cleavage sites can be between the IL-18, a fragment of IL-18, an IL-18 variant, or a fragment of the IL-18 variant and the first protein capable of translocating into an endoplasmic reticulum (ER) or fragment thereof, or within the PP, between PP or the PP variant and the IL-18, a fragment of IL-18, an IL-18 variant, or a fragment of the IL-18 variant, or within the PP, between the PP or the PP variant and the first protein capable of translocating into an endoplasmic reticulum (ER) or fragment thereof, or within the IL-18, a fragment of IL-18, an IL-18 variant, or a fragment of the IL-18 variant, or within the PP, or a combination thereof.

In various embodiments, the fusion protein can further comprise a second protein capable of translocating into the ER or a fragment thereof, wherein the second protein capable of translocating into the ER can be on the C-terminus end relative to the interleukin 18 (IL-18), the fragment of the IL-18, the IL-18 variant, or the fragment of the IL-18 variant.

In various embodiments, the fusion protein can further comprise a cleavage site between the IL-18, a fragment of IL-18, an IL-18 variant, or a fragment of the IL-18 variant and the second protein capable of translocating into an endoplasmic reticulum (ER) or fragment thereof, wherein the cleavage site can be cleaved by a protease.

In various embodiments, the fusion protein can comprise the interleukin 18 (IL-18), the fragment of the IL-18, the IL-18 variant, or the fragment of the IL-18 variant fused to the C-terminus of the first protein capable of translocating into the ER.

In various embodiments, the fusion protein can comprise the second protein capable of translocating into the ER or a fragment thereof fused to the C-terminus of the interleukin 18 (IL-18), the fragment of the IL-18, the IL-18 variant, or the fragment of the IL-18 variant.

In various embodiments, the fusion protein can comprise the IL-18 variant having diminished binding to IL-18 binding protein (IL-18BP), as compared to a wild type (wt) IL-18. In various embodiments, the fusion protein can comprise the IL-18 variant having a binding affinity to the human IL-18 receptor (IL-18R) within 30-fold of the wild-type IL-18. In various embodiments, the ratio of binding affinity of the fusion protein comprising the IL-18 variant to IL-18BP: binding affinity of the fusion protein comprising the IL-18 variant to IL-18R can be no higher than 3:1.

In various embodiments, the first protein capable of translocating into the ER or a fragment thereof of the fusion protein can be a globular protein, immunoglobular protein, or a fragment thereof, or can be a short polypeptide or protein engineered with a signal peptide for translocating into the ER, optionally the short polypeptide or protein can be about 2 kDa or no greater than 250 kDa, or optionally, the short polypeptide can be 1, 2, 3, or 4 amino acids, or being a flexible linker or amino acid chain of 5, 6, 7, 8, 9, 10, 11, 12, 13, 14, 15, 16, 17, 18, 19, 20, 25, 30, or 35 amino acids.

In various embodiments, the first protein capable of translocating into the ER or a fragment thereof of the fusion protein can be selected from the group consisting of a fragment crystallizable (Fc) region, human serum albumin (HSA), beta2microglobulin, transferrin, fragment antigen-binding region (Fab region), VHH antibody, single-chain variable fragment (scFv), anticalin, designed ankyrin repeat protein (DARPin), a binding domain thereof, and a fragment thereof.

In various embodiments, the first protein capable of translocating into the ER or a fragment thereof of the fusion protein can be a type I transmembrane protein or a fragment thereof. In various embodiments, the first protein capable of translocating into the ER or a fragment thereof of the fusion protein can be a type II transmembrane protein or a fragment thereof.

In various embodiments, the second protein capable of translocating into the ER or a fragment thereof of the fusion protein can be a globular protein, immunoglobular protein, or a fragment thereof, or can be a short polypeptide or protein engineered with a signal peptide for translocating into the ER, optionally the short polypeptide or protein can be about 2 kDa or no greater than 250 kDa.

In various embodiments, the second protein capable of translocating into the ER or a fragment thereof of the fusion protein can be selected from the group consisting of a fragment crystallizable (Fc) region, human serum albumin (HSA), beta2microglobulin, transferrin, fragment antigen-binding region (Fab region), VHH antibody, single-chain variable fragment (scFv), anticalin, designed ankyrin repeat protein (DARPin), a binding domain thereof, and a fragment thereof.

In various embodiments, the second protein capable of translocating into the ER or a fragment thereof of the fusion protein can be a type I transmembrane protein or a fragment thereof.

In various embodiments, the second protein capable of translocating into the ER or a fragment thereof of the fusion protein can be a type II transmembrane protein or a fragment thereof.

In various embodiments, the Fc region of the fusion protein can be an Fc region from IgA, IgM, IgG, or IgE. In various embodiments, the Fc region of the fusion protein can be an Fc region from IgG4, KiH, or IgG1. In various embodiments, the Fc region of the fusion protein can be an Fc region from Knob-in-hole, HA-TF, Xmab, ZW1, 7.8.60. Electrostatic Steering, DD-KK. EW-RVT, A107, or Duobody.

In various embodiments, one or more cysteines in the fusion protein can be modified. In various embodiments, one or more cysteines in the fusion protein can be replaced with a natural or non-natural amino acid (other than cysteine). In various embodiments, one or more cysteines in the IL-18, the fragment of IL-18, the IL-18 variant, or the fragment of the IL-18 variant of the fusion protein can be modified or can be replaced with a natural or non-natural amino acid (other than cysteine). In various embodiments, the one or more cysteines in the PP or PP variant of the fusion protein can be modified or can be replaced with a natural or non-natural amino acid (other than cysteine). In various embodiments, the natural amino acid can be charged, polar uncharged, or hydrophobic. In various embodiments, the natural amino acid can be each independently selected from serine and valine. In various embodiments, the natural amino acid can be each independently selected from threonine, asparagine, and glutamine. In various embodiments, the natural amino acid can be each independently selected from alanine, isoleucine, leucine methionine, phenylalanine, tyrosine, and tryptophan. In various embodiments, the natural amino acid the natural amino acid can be each independently selected from phenylalanine, alanine, aspartic acid, and asparagine. In various embodiments, the natural amino acid can be valine. In various embodiments, the natural amino acid can be each independently selected from threonine, glutamine, aspartic acid, phenylalanine, isoleucine and histidine.

In various embodiments, the protease can be selected from the group consisting of EK, TEV, Adam17, cathepsin, MMP2, MMP9, MMP14. Granzyme A, Granzyme B, Granzyme M, Granzyme K, and combinations thereof.

In various embodiments, the fusion protein can further comprise a tumor associated antigen binding domain. In various embodiments, the fusion protein can further comprise a binding domain for a protein expressed on immune cells. In various embodiments, the fusion protein can further comprise a binding domain for a protein expressed on immune cells that express IL-18 receptor complex or on immune cells that upon activation express the IL-18 receptor complex.

In various embodiments, the fusion protein can comprise one or more sequences as set forth in any one in Tables 1A, 1B, and 4.

Various embodiments provide for a fusion protein comprising a mature IL-18 variant selected from Table 1B; and a short polypeptide. In various embodiments, the short polypeptide can be any one of the short polypeptides described herein.

In various embodiments, the IL-18 variant can be from FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116. FUSE-1176, or FUSE-1177.

Various embodiments provide for an IL-18 variant selected from Table 1B corresponding to FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177.

Various embodiments provide for a fusion protein selected from the group consisting of FUSE-557. FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1M depicts illustrations of formats for pro-IL-18 fusion proteins. All proteins incorporate a human IgG scaffold that is either wild type homodimeric IgG1 or comprising a knob-in-hole (KIH) heterodimeric IgG1 Fc. Pro-IL-18, when included in the fusion protein is fused as (a) a singular cassette to either the C-terminal end of the knob (or hole) chain or (b) as singular cassettes to both the C-terminal ends of the knob chain and the hole chain. Cleavage sites, when present in the pro-IL-18 cassette, are indicated as a star shape. A tumor associated antigen binding domain (TAA), when present, is fused as a singular cassette to the N-terminal end of a knob (or hole) chain, or both chains of the KIH heterodimer IgG Fc. The TAA binding domain may be a VHH (e.g. EGFR specific clone 9G8) or a fab (e.g. EGFR specific clone C225 from cetuximab).

FIG. 2A-2B depicts the functional activity as it relates to Antibody Dependent Cellular Cytotoxicity (FIG. 2A) and IFNγ release (FIG. 2B) induced by FUSE556 (single armed anti-EGFR VHH 9G8-IgG1), and FUSE686 (single armed anti-EGFR VHH 9G8-IgG1-pro-IL-18).

FIG. 3A-3B depicts the functional activity as it relates to Antibody Dependent Cellular Cytotoxicity (FIG. 2A) and IFNγ release (FIG. 2B) induced by of FUSE556 (single armed anti-EGFR VHH 9G8-IgG1; targets both EGFR and FcγR), or the combination of FUSE556 and FUSE422 (IgG1-pro-IL-18; only targets FcγR).

FIG. 4 depicts the functional activity as it relates IFNγ release induced by of FUSE691 (nivolumab), FUSE645 (EGFRtargeted IgG1-pro-IL-18; non-targeted in this system), FUSE694 (nivolumab-pro-IL-18) and human recombinant IL-18 on T cell/allogeneic DC co-culture.

FIG. 5A-5B depicts the functional activity as it relates to Antibody Dependent Cellular Cytotoxicity of FUSE556 (single armed anti-EGFR VHH 9G8-IgG1), and FUSE516 (single armed anti-EGFR VHH 9G8-IgG1-pro-IL-18). Graphs illustrating killing of EGFR+ MDA-MB-231 tumor cells by PBMC from each of four normal human donors are shown in FIGS. 5A-5D.

FIG. 6A-6B depicts the functional activity as it relates to pro-IL-18 fusion protein induced Antibody Dependent Cellular Cytotoxicity (FIG. 6A, left panel), IFNγ release (FIG. 6A, right panel), Granzyme B release (FIG. 6B, left panel), and IL-18 release/consumption (FIG. 6B, right panel) after 48 hours of co-culture of PBMC and EGFR+ MDA-MB-231 tumor cells. The pro-IL-18 fusion proteins tested were FUSE556 (single armed anti-EGFR VHH 9G8-IgG1), and FUSE516 (single armed anti-EGFR VHH 9G8-IgG1-pro-IL-18).

FIG. 7A-7B depicts the functional activity as it relates to Antibody Dependent Cellular Cytotoxicity induced by FUSE556 (single armed anti-EGFR VHH 9G8-IgG1), variants of pro-IL-18 fused to the C-terminus of FUSE566 with different protease cleavage sites between the Fc domain and pro-IL-18 (FIG. 7A), and a variant that contains the PGLALA mutation in its Fc domain to abolish targeting to FcγRs (FUSE627; FIG. 7B). The protease sites tested were Granzyme A (FUSE658), Granzyme B (FUSE516), MMP2/9 (FUSE659), and the combination of Granzyme A, Granzyme B and MMP2/9 (FUSE660).

FIG. 8A-8D depicts the functional activity as it relates to pro-IL-18 fusion protein induced Antibody Dependent Cellular Cytotoxicity (FIGS. 8A, 8C) and IFNγ release (FIGS. 8B-8D) after 48 hours of co-culture of expanded NK cells and EGFR+ MDA-MB-231 tumor cells at E:T ratios of 1:1 (FIGS. 8A and 8C) and 1:5 (FIGS. 8C and 8D The pro-IL-18 fusion proteins tested were FUSE556 (single armed anti-EGFR VHH 9G8-IgG1), and FUSE516 (single armed anti-EGFR VHH 9G8-IgG1-pro-IL-18).

FIG. 9A-9B depicts the functional activity as it relates to pro-IL-18 fusion protein induced Antibody Dependent Cellular Cytotoxicity (FIG. 9A) and IFNγ release (FIG. 9B) after 48 hours of co-culture of expanded NK cells and EGFR+ MDA-MB-231 tumor cells at E:T ratios of 2:1. The pro-IL-18 fusion protein tested was FUSE555 (dual armed anti-EGFR Fab C225-IgG1-pro-IL-18), which was compared to cetuximab.

FIG. 10A-10B depicts the functional activity as it relates to pro-IL-18 fusion protein induced Antibody Dependent Cellular Cytotoxicity (FIG. 10A) and IFNγ release (FIG. 10B) after 48 hours of co-culture of expanded NK cells and HER2+ T47D tumor cells at E:T ratios of 5:1. The pro-IL-18 fusion proteins tested were FUSE624 (dual armed anti-HER2 Fab IgG1-pro-IL-18) and trastuzumab (Herceptin).

FIG. 11 (panels A-E) depicts exemplary fusion proteins in which the N-terminus of pro-IL-18 is fused to the C-terminus of the knob of a knob-into-hole heterodimeric IgG1 protein, which has a structure from N- to C-terminus comprising knobs-in-hole (KiH) Fc-propeptide (PP)-enterokinase-cleavable site (EK)-IL-18 wild type or its variants. This depicts exemplary fusion proteins such as IDs: FUSE-480, FUSE-481, and FUSE-442 in Table 1A. Further modification was made to pro-IL-18 to reduce aggregation of the molecule, wherein each cysteine residue in both the pro-peptide and mature IL-18 was replaced with serine (as in FUSE-480, denoted as “IL-18AS”), with alanine (as in FUSE-481, denoted as “IL-18AA”), or with valine (as in FUSE-442, denoted as “IL-18AV”). Alternatively, the N-terminus of pro-IL-18 can be fused to the C-terminus of the hole chain of a KiH heterodimeric IgG1 proteins. The biological activity defined as the EC50-SEAP for each compound is shown in panel E.

FIG. 12 (panels A-E) depicts exemplary fusion proteins in which the N-terminal of pro IL-18 was fused to the C-terminal of an IgG1 CH3 domain (which is also a knob chain of a knob-into-hole heterodimeric IgG1 protein as in FIG. 1), and the pro IL-18 incorporated four amino acid substitutions hypothesized to reduce binding to IL-18BP while maintaining wild type binding to the IL-18 receptor complex, denoted as “pro-IL-18mut2”. These fusion proteins have a structure from N- to C-terminus comprising knobs-in-hole (KiH) Fc-propeptide (PP)-enterokinase-cleavable site (EK)-IL-18mut2. Further modification was made to the pro-IL-18mut2 to reduce aggregation of the molecule, wherein each cysteine residue in both the pro-peptide and mature IL-18mut2 was substituted with serine (denoted as “IL-18mut2AS”, as in FUSE-422; panel B), with alanine (denoted as “IL-18mut2AA”, as in FUSE-423; panel C), or with valine (denoted as “IL-18mut2AV”, as in FUSE-424: panel D). The biological activity defined as the EC50-SEAP for each compound is shown in panel E.

FIG. 13 (panels A-D) depicts exemplary fusion proteins with (panel A) or without (panel B) the propeptide to examine the impact on masking of “IL-18AV” biological activity (panel C), wherein Fc fusion variants were generated incorporating “IL-18AV” with the propeptide (panel A; FUSE-442) or without (panel B: FUSE-505) the propeptide. The biological activity defined as the EC50-SEAP for each compound is shown in panel D.

FIG. 14 (panels A-D) depicts exemplary fusion proteins with (panel A) or without (panel B) the propeptide to examine the impact of propeptide on masking of “IL-18mut2AV” biological activity, wherein Fc fusion variants were generated incorporating “IL-18mut2AV” without the propeptide (hence, a mature IL-18 with mutation, denoted as “matIL-18mut2-AV”, see panel B; FUSE-441) or with the propeptide (panel A; FUSE-424). For a fusion protein devoid of the propeptide, the EK cleavage site that replaced the Caspase 1 site was moved to a position directly in between the CH3 domain of the knob and the mature IL-18AV without the addition of a flexible linker. Panel C depicts the activation readout using the HEK-Blue IL-18AV reporter cell assay following exposure to a titration of FUSE-441 (Fc-EK-IL-18AV) or FUSE-424 (Fc-EKpp-IL-18AV) with or without treatment with EK. The biological activity defined as the EC50-SEAP for each compound is shown in panel D.

FIG. 15 (panels A-D) depicts exemplary fusion proteins in which the N-terminus of pro-IL-18 is fused to the C-terminus of IgG1 Fc protein or IgG4 Fc; which has a structure from N- to C-terminus comprising IgG1 Fc-propeptide (PP)-IL-18AV (FUSE-507; panel A) and IgG4 Fc-propeptide (PP)-IL-18AV (FUSE-509; panel B). Panel C depicts the activation readout using the HEK-Blue IL-18AV reporter cell assay of exposing the cells to a titration of FUSE-507 or FUSE-509 with or without treatment with EK. The biological activity defined as the EC50-SEAP for each compound is shown in panel D.

FIG. 16 (panels A-E) depicts exemplary fusion proteins in which the N-terminus of pro-IL-18 is fused to the C-terminus of HSA with or without the propeptide (PP): which has a structure from N- to C-terminus comprising HSA-propeptide (PP)-IL-18AV (FUSE-501: panel A) and HSA-IL-18AV (FUSE-503: panel B). Panels C and D depict the activation readout. The biological activity defined as the EC50-SEAP for each compound is shown in panel E.

FIG. 17 (panels A-E) depicts exemplary fusion proteins in which the N-terminus of pro-IL-18mut2 is fused to the C-terminus of HSA with or without the propeptide (PP); which has a structure from N- to C-terminus comprising HSA-propeptide (PP)-IL-18mut2AV (FUSE-502; panel A) and HSA-IL-18mut2AV (FUSE-504; panel B). Panels C and D depict the activation readout. The biological activity defined as the EC50-SEAP for each compound is shown in panel E.

FIG. 18 (panels A-D) depicts exemplary fusion proteins in which the C-terminus of pro-IL-18 is fused to the N-terminus of the knob of a knob-into-hole heterodimeric IgG1 protein with or without the propeptide (PP); which has a structure from N- to C-terminus comprising propeptide (PP)-IL-18AV-knobs-in-hole (KiH) Fc (FUSE-499; panel A) and IL-18AV-knobs-in-hole (KiH) Fc (FUSE-500; panel B). Panel C depicts the activation readout. The biological activity defined as the EC50-SEAP with or without exposure to Caspase 1 for each compound is shown in panel D.

FIG. 19A-19I depicts exemplary fusion proteins with a structure from N- to C-terminus: Fc-ppMMP2/9-cleavage sites-IL-18-AV (FUSE-486) or Fc-ppMMP9/2-cleavage sites-IL-18-AV (FUSE-487), in which the cleavage sites are specific for the metalloproteases, MMP2 and MMP9, with preferred enzyme to the left of the forward-slash, or FUSE-485 (Fc-GzmBpp-IL-18AV) and FUSE-462 (Fc-GzmBpp-IL-18mut2AV). FIG. 19B depicts the activation readout relating to FUSE-486 and FUSE-487, with or without MMP2 treatment. The biological activity defined as the EC50-SEAP for each of FUSE-486 and FUSE-487, with or without MMP2 treatment, is shown in FIG. 19B. FIG. 19C shows that FUSE587 was about 3,000-fold attenuated relative to recombinant human IL-18. Interestingly, we observed that cleavage of FUSE587 with MMP2 released and IL-18AV variant that was still about 100-fold attenuated relative to recombinant IL-18. In contrast, cleavage with Granzyme B released an IL-18AV variant with activity similar activity as recombinant IL-18. Cleavage with Granzyme B results in release of mature IL-18AV without any N-terminal residues constituting an overhang, whereas 11 and 15 amino acid N-terminal polypeptide overhangs remain after cleavage of FUSE486 and FUSE587, respectively, with MMP2. We speculated that these overhangs might be attenuating IL-18AV activity, albeit to a lesser degree than the full size variant propeptide. This phenomenon was further investigated in FIG. 11 and FIG. 13. FIGS. 19D and 19G also depicts exemplary fusion proteins with a structure from N- to C-terminus: Fc-ppGb-cleavage sites-IL-18-AV (FUSE-485; 19D) or Fc-ppGb-cleavage sites-IL-18mut2-AV (FUSE-462; 19D) or Fab-Cetuximab-Fc-ppGb-cleavage sites-IL-18-AV (FUSE-517; 19G), in which the cleavage site is specific for granzyme B (Gb). FIG. 19 E depicts the activation readout relating to FUSE-462 and FUSE-485, with or without granzyme B treatment. FIG. 19F shows the biological activity defined as the EC50-SEAP for each of FUSE-462 and FUSE-485, with or without granzyme B treatment. FIG. 19H depicts the activation readout relating to FUSE-517, with or without enzyme treatment. FIG. 19I shows the biological activity defined as the EC50-SEAP for FUSE-517, with or without granzyme B treatment.

FIG. 20 (panels A-G) depicts the impact of IL-18BP on the biological activity of recombinant human IL-18 (rhIL-18) and the EK cleavage products of the exemplary fusion proteins, Fc-ppEK-IL-18-AV (FUSE-442) and Fc-ppEK-IL-18mut2AV (FUSE-424). The biological activity defined as the EC50-SEAP for each compound with and without the addition of IL-18BP (competition assay) is shown in panels B, D and F, with corresponding biological activity defined as the EC50-SEAP shown in panels C, E, and G, respectively.

FIG. 21A depicts diagrams of exemplary fusion proteins in which the C-terminus of pro-IL-18 is fused to the N-terminus of the knob of a knob-into-hole heterodimeric IgG1 protein with different size polypeptides fused to the N-terminus of mature IL-18. FIG. 21B depicts the biological activity of each fusion protein using the IL-18 reporter cell line, HEK-Blue IL-18.

FIGS. 22A, 22B(i), 22B(ii), 22C, 22D(i), 22D(ii) and 22E depict human IL-18 engineered mutant fusion proteins in accordance with various embodiments of the invention.

FIG. 23 shows the impact of the size of polypeptides fused to the N-terminus of mature IL-18 on the biological activity of a single IL-18AV fused to the N-terminus of IgG1 Fc.

FIG. 24A-24H shows the impact of the substituting the cysteine residue in the pro-peptide and cysteine residues in the mature IL18, which were fused together to form the pro-IL-18 variant cassette, on the biological activity of each variant using the HEK Blue IL18 assay system.

FIG. 25A-25B shows the impact of targeting pro-IL18 to within close proximity of its receptor complex (i.e., “cis activity”).

FIG. 26 shows the assessment of the capacity of an anti-PDL1 antibody incorporating a pro-IL-18amut2 variant, designed so that it cannot be cleaved by Granzyme B, to induce T cell mediated IFNγ release. A summary table of EC50, Emax and AUC (area under the curve) is shown beneath of x-y plot.

FIG. 27A-27B shows the assessment of the capacity of an anti-PD1 antibody, incorporating one of two pro-IL-18 variants (pro-IL18amut2 or pro-IL18amut9), designed so that it cannot be cleaved by Granzyme B, to induce T cell mediated tumor cell killing (FIG. 27A) and IFNγ release (FIG. 27B). A summary table of EC50. Emax and AUC (area under the curve) is shown beneath each x-y plot.

FIG. 28 shows the capacity of several IL-18 variants to induce non-targeted trans based release of SEAP from HEK-Blue-IL-18.

FIG. 29A-29B shows the capacity of several IL-18 variants targeted to human PD-1 to induce cis based release of IFNγ from human PBMC derived from a healthy human donor (FIG. 29A) or human T cells (FIG. 29B).

FIG. 30A-30B shows the capacity of an exemplary IL-18 variant targeted to mouse PD-1 to mediate tumor growth inhibition of an aggressive syngeneic melanoma tumor in a fully immunocompetent mouse tumor model.

FIG. 30C show the number of different lymphocyte subsets per gram of tumor was assessed from tumor bearing mice treated with PBS (light grey), anti-PD1 (black), and Fuse 1113 (horizontal lines).

FIG. 30D depicts the frequency of total myeloid cells (CD11b+ cells), M2 macrophages (CD11b+ F4-80+CD11c-CD206+ cells) and the ratios of CD8+ T cells to total Myeloid Derived Suppressor Cells (MDSC: CD11b+Ly6C+Ly6G-[M-MDSC] and CD11b+Ly6C-mid Ly6G+[G-MDSC]), CD8⁺ T cells to M2 macrophages, anti-PD-1 responsive CD8+ T cells to MDSC, and CD8+ T cells to CD4+ T cells.

FIG. 31A-31B shows the capacity of an exemplary IL-18 variant targeted to mouse PD-1 to mediate tumor growth inhibition in a syngeneic colorectal cancer tumor in a fully immunocompetent mouse tumor model.

FIG. 32A-32B shows the capacity of an exemplary IL-18 variant targeted to mouse PD-1 to mediate tumor growth inhibition in a syngeneic rapidly growing colorectal cancer tumor in a fully immunocompetent mouse tumor model.

FIG. 33A shows C57BL/6 mice injected subcutaneously with MC38i. Serum was collected at 0, 24, 72 and 144 hours after the Day 0 treatment and mouse IFNγ measured.

FIG. 33B shows C57BL/6 mice that harbored MC38i tumors and experienced a CR following treatment with αPD1-HT18cis or αPD-1-IL-18 were re-challenged with MC38i after 90 days.

FIG. 34 shows BALB/c mice that harbored CT26.C tumors and experienced a CR following treatment with αPD1-HT18cis were rechallenged with CT26.C after 65 days on the left flank and parental CT26 after 70 days on the right flank. As a means of comparison, a group of 5 naïve mice were subjected to the equivalent tumor inoculation.

FIG. 35 shows C57BL/6 mice were injected subcutaneously with B16-F10R. When mean tumor volume ranged between 75-100 mm3, mice were randomized into groups of 5 and treated with PBS, αPD-1, αPD1-HT18cis on Days 0, 3 and 6 at 15 mg/kg. Mice that experienced a CR following treatment with αPD1-HT18cis were rechallenged with B16-F10 after 60 days.

FIG. 36 shows C57BL/6 mice harboring B16-F10R tumors were treated on Days 0 and 3. Mice were euthanized on Day 5, tumors collected and dispersed into singe cell suspensions for flow cytometric assessment of TIL (CD45+ cells). TEM and TCM were defined CD44+/CD62L− and CD44+/CD62L−, respectively within the CD8+ pool. αPD-1 responsive CD8+ T cells were defined as PD1+/TCF1+ cells within the CD8+ pool.

FIG. 37 shows C57BL/6 mice harboring B16-F10R tumors were treated on Days 0 and 3. Mice were euthanized on Day 5, tumors collected and dispersed into singe cell suspensions for flow cytometric assessment of TIL (CD45+ cells). All myeloid subsets were gated on CD11b. Within that pool, MDSC were defined as Ly6G+/Ly6Cmid+Ly6G-/Ly6Chigh and M2 macrophages as F480+/CD206+.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed, J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed (Cold Spring Harbor Press, Cold Spring Harbor NY, 2013); Kohler and Milstein, (1976) Eur. J. Immunol. 6: 511; Queen et al. U.S. Pat. No. 5,585,089; and Ricchmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” or “approximately” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “immunoglobulin heavy chain constant region” is used interchangeably with the term “Fc region” and is understood to mean the carboxyl-terminal portion of an immunoglobulin heavy chain constant region, or an analog or portion thereof capable of binding an Fc receptor. Each immunoglobulin heavy chain constant region comprises four or five domains. The domains are named sequentially as follows: CH1-hinge-CH2-CH3(-CH4). CH4 is present in IgM, which has no hinge region. The immunoglobulin heavy chain constant region suitable for the invention preferably comprises an immunoglobulin hinge region, and preferably also includes a CH3 domain. The immunoglobulin heavy chain constant region most preferably comprises an immunoglobulin hinge region, a CH2 domain and a CH3 domain.

As used herein, the term immunoglobulin “hinge region” is understood to mean an entire immunoglobulin hinge region or at least a portion of the immunoglobulin hinge region sufficient to form one or more disulfide bonds with a second immunoglobulin hinge region.

As used herein, the term “vector” is understood to mean any nucleic acid comprising a nucleotide sequence competent to be incorporated into a host cell and to be recombined with and integrated into the host cell genome, or to replicate autonomously as an episome. Such vectors include linear nucleic acids, plasmids, phagemids, cosmids, RNA vectors, viral vectors and the like. Non-limiting examples of a viral vector include a retrovirus, an adenovirus and an adeno-associated virus.

As used herein, the term “gene expression” or “expression” of a fusion protein, is understood to mean the transcription of a DNA sequence, translation of the mRNA transcript, and secretion of a fusion protein product. In some embodiments, the expression process also includes or is followed by purification; for example, protein A affinity chromatography or other means such as size exclusion chromatography can be used for purification.

As used herein, the term “cis” in the context of interactions refers to the interaction between two molecules on the same cell, such as proteins expressed on the same cell. A nonlimiting example is the interaction between B7-1 and PD-L1. “Cis” interaction as used herein also includes multispecific proteins that bind two proteins on the same cell simultaneously.

As used herein, the term “trans” in the context of interactions refers to the interaction between two molecules on different cells, such as proteins on different cells. A nonlimiting example is the interaction between the prototypical interaction of the T cell Receptor (TCR) and its ligand, an MHC-I/peptide complex. “Trans” interaction as used herein also include multispecific proteins that bind one protein on one cell and a second protein on a separate cell; hence a “bridge” would be considered as occurring in trans. Also included as a “trans” interaction is an interaction between a soluble protein and a cell surface protein, although not forming a cellular bridge.

As used herein, “IL-18 fusion protein” refers to a fusion protein that includes wild-type IL-18 or IL-18 variants, unless specifically noted as only including the wild-type IL-18, or only including the IL-18 variant. Thus, in particular embodiments, the “IL-18 fusion protein” only includes any one of the IL-18 variants as described herein.

The term “linker” with respect to amino acid linker in a polypeptide can be a short peptide. Examples of these short peptides include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5 In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5. (GGGX_{λ})ₙ wherein X_{λ} is Q, A, E or S and n=1-5 or in some embodiments, an integer larger than 5. Still additional examples include (X_{λ}GG)ₙ wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5. In some embodiments, the amino acid linker is an IL-18 propeptide or IL-18 propeptide variant. In some embodiments, the amino acid linker is a fragment of an IL-18 propeptide or IL-18 propeptide variant: for example, about 30-36 amino acids in length, about 5-10, 11-20, 21-30, or 31-40 amino acids in length. As further examples, the linker can be a short polypeptide of 2, 3, or 4 amino acids in length, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids.

This invention relates to the applications of IL-18 fusion proteins. This includes antibody and antibody-like fusion proteins that are targeted to proteins expressed on immune cells (e.g., NK cells, T cells, neutrophils, mast cells), tumor cells and/or cells in the tumor microenviromnent. The fusion protein incorporates an IL-18 or IL-18 variant with reduced binding affinity for its natural antagonist. IL-18-Binding-Protein, while maintaining the binding affinity to its receptor. The activity of the IL-18 variant may be attenuated or masked such that it has little to no activity in trans but can still bind to and activate the IL-18 receptor complex (RC) in cis. That is, when the attenuated or masked variant of IL-18 is fused to a protein that delivers the aforementioned protein to another protein on the same cell surface as the IL-18 RC, the context of being in close proximity allows for the cytokine to transmit a signal.

In addition, release of the mask is mediated by proteases for which the cleavage sites are added in a linker between IL-18 and another domain of the fusion protein. Protease sites include metalloproteases expressed in the tumor microenvironment and/or granzymes released by cytotoxic immune cells. Masked IL-18 fusion proteins incorporating a granzyme cleavage site in the linker sequence that are targeted to both tumor(s) and an activation receptor on immune cell(s) can be used to treat cancer. The bridge formed by the IL-18 fusion protein between the tumor and immune activation receptor induces release of granzymes with the capacity to not only kill tumor cells but also release fully active IL-18 or the IL-18 variant that is fully functional in trans. As such, depending on context, masked IL-18 fusion proteins have the capacity to function in cis and trans. Since IL-18 or the IL-18 variant activates the targeted immune cells to proliferate, become more cytotoxic and produce more granzymes (see e.g., El-Darawish et al., J. Leukoc. Biol. 2018), a positive feedback loop can ensue. Thus, a greater number of more cytotoxic immune cells become available for bridging. These immune cells have a greater capacity to kill tumors and release a larger amount of granzymes and IL-18 or IL-18 variant to further amplify the response. Release of IL-18 or the IL-18 variant would be expected to be most efficient within the immune cell/tumor cell synapse because masked IL-18 localizes in this space and degranulation occurs in the same synapse.

Tumor reactive pathways can be directly built into the IL-18 fusion protein and include Antibody Dependent Cellular Cytotoxicity (ADCC), activation receptor mediated redirected cytotoxicity, co-stimulation and immune checkpoint blockade. IL-18 can also support additional innate and adaptive tumor specific immune responses including antigen presentation by dendritic cells, survival of tumor reactive NK cells and T cells (jlb.onlinelibrary.wiley.com/doi/full/10.1002/JLB.IHI1017-396RR), reduction in collagen deposition into the extracellular space (pubmed.ncbi.nlm.nih.gov/19865096) and support of immune memory (www.ncbi.nlm.nih.gov/pmc/articles/PMC2408965).

### Methods of Using the Fusion Protein.

Various embodiments of the invention provide for a method of treating cancer in a subject in need thereof, comprising: administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of treating cancer in a subject in need thereof, comprising: administering a fusion protein to a subject in need thereof wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of activating an IL-18 receptor complex (RC) signal on a cell, comprising: administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the subject has or is suspected to have cancer, or wherein the subject has one or more symptoms of cancer. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of activating an IL-18 receptor complex (RC) signal on a cell, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of releasing or exposing mature IL-18 in a tumor microenvironment or adjacent to a tumor, comprising: administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant. In various embodiments, the first polypeptide is not the wild-type IL-18 propeptide, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the protein capable of translocating into an endoplasmic reticulum (ER) or a fragment thereof is not the wild-type IL-18 propeptide. In various embodiments, the subject has or is suspected to have cancer, or wherein the subject has one or more symptoms of cancer. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of releasing or exposing mature IL-18 in a tumor microenvironment or adjacent to a tumor, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of increasing activity, persistence and immune memory of CD8⁺ T cells, CD4⁺ T cells, or γδ T cells, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the subject has or is suspected to have cancer, or wherein the subject has one or more symptoms of cancer. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of increasing activity, persistence and immune memory of CD8⁺ T cells, CD4⁺ T cells, or γδ T cells, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of reducing collagen deposition into extracellular space, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant. In various embodiments, the first polypeptide is not the wild-type IL-18 propeptide, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the protein capable of translocating into an endoplasmic reticulum (ER) or a fragment thereof is not the wild-type IL-18 propeptide. In various embodiments, the subject has or is suspected to have fibrosis, or the subject has one or more symptoms of fibrosis. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of reducing collagen deposition into extracellular space, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Many tumors, such as pancreatic cancer, are highly fibrotic. It is very difficult for lymphocytes to enter these tumors. Therefore, a therapeutic that can suspend or reduce tumor fibrosis can aid in the infiltration of lymphocytes into the tumor microenvironment. IL-18 has been reported to block collagen formation by fibroblasts and participate in the reduction of fibrosis. TGF beta is the strongest cytokine mediator of fibrosis and IL-18 can antagonize such activity. TGF beta is a key tumor immunosuppressive factor. Interestingly, Discoidin Domain Receptor (DDR1), often expressed on tumor cells recognizes collagen. It is known to contribute to tumor lymphocyte exclusion by collagen fiber alignment. It has also recently been shown to inhibit IL-18 synthesis and upregulate PDL1.

Accordingly, various embodiments of the invention provide for a method of treating tumor related fibrosis, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of treating tumor related fibrosis, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of reducing or inhibiting fibrosis in a tissue, comprising administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Various embodiments of the invention provide for a method of reducing or inhibiting fibrosis in a tissue, comprising: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

In some embodiments, the fibrosis is in a tumor tissue, and hence a method of reducing or inhibiting fibrosis in a tumor tissue is provided, which includes administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

In some embodiments, a method of reducing or inhibiting fibrosis in a tumor tissue comprises: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

In some embodiments, the fibrosis is in a tumor tissue expressing discoidin domain receptor (DDR1), and hence a method of reducing fibrosis in a DDR1+ tumor is provided, which includes administering a fusion protein to a subject in need thereof, wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the fusion protein comprises a first polypeptide or protein capable of translocating into an endoplasmic reticulum (ER) or a fragment thereof; and an IL-18 variant. In various embodiments, the first polypeptide is not the wild-type IL-18 propeptide. In various embodiments, the protein capable of translocating into an endoplasmic reticulum (ER) or a fragment thereof is not the wild-type IL-18 propeptide. In various embodiments, the method further comprises administering an immunotherapeutic agent. In various embodiments, the immunotherapeutic agent is immune check point inhibitor.

In some embodiments, a method of reducing fibrosis in a DDR1+ tumor comprises: administering a fusion protein to a subject in need thereof, wherein the fusion protein comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. In various embodiments, the fusion protein comprises (i) an IL-18 variant and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum. In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). Additional details of the fusion proteins are provided herein. In various embodiments, the method further comprises administering an immunotherapeutic agent. In various embodiments, the immunotherapeutic agent is immune check point inhibitor. In various embodiments, the immunotherapeutic agent comprises an agent selected from Table 5.

Examples of immune check point inhibitors used in accordance with these methods include but are not limited to pembrolizumab, nivohunab, pidilizumab, AMP-514, spartalizumnab, cemiplimab, AK105, BCD-100, BI 754091, JS001, LZM009, MGA012, Svm021, TSR-042, MGD013, AK104, XmAb20717, tislelizumab, and PF-06801591.

In various embodiments, the immunotherapeutic agent comprises an agent selected from Table 5.

The fusion proteins in accordance with various embodiments of methods of using the fusion proteins are further described herein. Thus, in various embodiments, the method of using the fusion proteins described above utilizes the fusion proteins described in more detail herein.

In various embodiments, the fusion proteins according to the invention are in pharmaceutical compositions formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, term alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o, or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Fusion Proteins Used in the Various Methods of the Invention

In accordance with the methods of the present invention for using the fusion proteins, fusion protein used in the methods are described herein.

As discussed above, the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant, wherein the IL-18, the fragment of IL-18, the IL-18 variant, or the fragment of the IL-18 variant is on the C-terminus end of the fusion protein relative to the first polypeptide or protein capable of translocating into the ER. In various embodiments, the first polypeptide is not the wild-type IL-18 propeptide. In various embodiments, the protein capable of translocating into an endoplasmic reticulum (ER) or a fragment thereof is not the wild-type IL-18 propeptide.

In addition to these features, additional features of the fusion protein are discussed herein.

In some embodiments, the fusion protein comprises an IL-18 variant. In various embodiments, the IL-18 variant has an amino acid sequence comprising or consisting of amino acid positions 37-193 of SEQ ID NO:250 with one to five amino acid substitutions at positions E42, M87, K89, M96, and M149 of MAAEPVEDNCINFVAMKFIDNTLYFIAEDDENIEQDYFGKLESKLSVIRNLNDQVLFIDQGNRPLFEDMT DSDVRDNAPRTIFIISMYKDSQPRGMAVTISVKVEKISTLSVENKIISFKEMNPPDNIKDTKSDIIFFQRSVP GHDNKMQFESSSYEGYFLAVEKERDLFKLILKKEDELGDRSIMFTVQNED (SEQ ID NO:250). In various embodiments, the one to five amino acid substitutions is one amino acid substitution. In other embodiments, the one to five amino acid substitutions are two amino acid substitutions. In other embodiments, the one to five amino acid substitutions are three amino acid substitutions. In other embodiments, the one to five amino acid substitutions are four amino acid substitutions. In other embodiments, the one to five amino acid substitutions are five amino acid substitutions. In various embodiments, the IL-18 variant comprises no more than five amino acid substitutions, with the exception of substituting cysteines.

In various embodiments, the IL-18 variant has an amino acid sequence comprising or consisting of amino acid positions 37-193 of SEQ ID NO:251 with one to five amino acid substitutions at positions E42, M87, K89, M96, and M149 of

(SEQ ID NO: 251)
MAAEPVEDNCINFVAMKFIDNTLYFIAEDDENIEQDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNED.

In various embodiments, the IL-18 variant has an amino acid sequence comprising or consisting of amino acid positions 37-193 of SEQ ID NO:251 with one or more amino acid substitutions at positions C74, C104, C112, and C164, and one to five amino acid substitutions at positions E42, M87, K89, M96, and M149, of SEQ ID NO:251. In various embodiments, the amino acid substitutions at one or more of C74, C104, C112, and C164 are each independently substituted to valine, alanine or serine.

In various embodiments, the one to five amino acid substitutions are one or more of E42K, E42R. E42A, E42H, or E42Q; M87K, or M87-I; K89G, K89A, or K89E: M96L, or M96I; or M149V or M149I. In various embodiments, the one to five amino acid substitutions are E42K, E42R, E42A, E42H, or E42Q; M87K, or M87H; K89G, K89A, or K89E: M96L, or M96I; and M149V or M149I.

In various embodiments, the fusion protein comprises an IL-18 variant selected from Table 1B. In various embodiments, the fusion protein comprising the IL-18 variant selected from Table 1B, further comprises a propeptide having an amino acid sequence selected from Propeptide column in Table 1B, and optionally from the same row as the IL-18 variant. In various embodiments, the fusion protein comprising the IL-18 variant selected from Table 1B and propeptide selected from Table 1B, further comprises a cleavage peptide selected from Table 1B, and optionally from the same row as the IL-18 variant and propeptide. As a particular example, IEQD (SEQ ID NO:88) can be used.

In various embodiments, IL-18 variant is an IL-18 variant disclosed in U.S. Pat. No. 7,524,488, U.S. Patent Publication No. 2019/0070262, U.S. Patent Publication No. 2021/0015891, or PCT Publication No. WO 2022/038417, the IL-18 variants and sequences of each of these patent or publications of which are hereby incorporated by reference as though fully set forth.

In various embodiments, the fusion protein further comprises a targeting polypeptide. In some embodiments, the targeting polypeptide targets a protein on a cell surface, wherein the cell surface also has an IL-18 RC or the cell is capable of expressing the IL-18 RC. In various embodiments, the fusion protein binds to a cell having an IL-18 RC or capable of expressing the IL-18 RC upon activation of the cell, and activates the IL-18 RC signal.

In other embodiments, the targeting polypeptide targets a protein on a cell surface that does not have an IL-18 RC or the cell is not capable of expressing the IL-18 RC. The cell not having the IL-18 RC on its surface or not capable of expressing the IL-18 RC is in close proximity to a cell expressing the IL-18 RC or is capable of expressing the IL-18 RC. In other instances, the fusion protein can bring the cell not having the IL-18 RC on its surface or not capable of expressing the IL-18 RC into close proximity to a cell expressing the IL-18 RC or is capable of expressing the IL-18 RC.

In various embodiments, the targeting polypeptide comprises a tumor associated antigen binding domain.

In various embodiments, the fusion protein further comprises a binding domain for a protein expressed on immune cells. In various embodiments, the fusion protein further comprises a binding domain for a protein expressed on immune cells that express IL-18 receptor complex or on immune cells that upon activation express the IL-18 receptor complex.

In various embodiments, the fusion protein further comprises an antibody or antibody fragment. This enables the fusion protein to bind to a tumor cell, or to an immune cell or stromal cell in a tumor tissue, or to a tumor draining lymph node, or other secondary lymphoid organ. Examples of antibody fragments include Fc fragment, Fab fragment, Fv fragment, as well as others discussed herein.

In various embodiments, the fusion protein further comprises a masking domain. In some of these embodiments, the masking domain provides protection for IL-18, and IL-18 cannot be released by a protease. In some of these embodiments, a mature IL-18 or mature IL-18 variant can be released from a masking domain by a protease. In various embodiments, the protease is granzyme, which can be released from an immune cell. Examples of immune cells include but are not limited to an NK cell, a T cell, a neutrophil, or a mast cell. In various embodiments, the protease is a metalloprotease, which the metalloprotease can be expressed in a tumor microenvironment or tumor draining lymph node. Further examples of protease and types of granzymes are described herein. In various embodiments, the mature IL-18 increases the activity of NK cells or T cells, and optionally the activity being one or more of proliferation, survival, and cytotoxicity.

In various embodiments, the fusion protein further comprises half-life extending molecule. A nonlimiting example of a half-life extending molecule is a half-life extending polypeptide; for example, human serum albumin (HSA) or an HSA-binding fragment.

In various embodiments, the fusion protein has reduced activity as compared to wild-type IL-18 when not bound to a cell having the IL-18 RC. In various embodiments, the reduced activity is at least a 75% reduction in activity as compared to wild-type IL-18.

In some embodiments, a mature IL-18 variant on the C-terminus of the mask is attenuated. In various embodiments, the mature IL-18 variant is 10× to 1000× attenuated, 10×-100× attenuated, or 100×-1000× attenuated. For example, mature IL-18-mut13A is 100 fold attenuated.

In various embodiments, the fusion protein comprises polypeptide 1 and polypeptide 2 selected from Table 1A. In various embodiments, the fusion protein further comprises polypeptide 3 selected from Table 1A. In various embodiments, polypeptide 1 and polypeptide 2, and optionally polypeptide 3 is selected from the same row of Table 1A.

In various embodiments, the fusion protein comprises polypeptide 1 selected from Table 1A, wherein polypeptide 1 comprises HSA.

In various embodiments, the fusion protein does not comprise an IL-18 variant disclosed in U.S. Pat. No. 7,524,488, U.S. Patent Publication No. 2019/0070262, U.S. Patent Publication No. 2021/0015891, or PCT Publication No. WO 2022/038417, the IL-18 variants and sequences of each of these patent or publications of which are hereby incorporated by reference as though fully set forth.

In various embodiments, each of the fusion proteins of the present invention comprises (i) an IL-18, a fragment of IL-18, an IL-18 variant, or a fragment of the IL-18 variant, and (ii) a first protein capable of translocating into an endoplasmic reticulum (ER) including a cytosolic or nuclear protein engineered to translocate into an endoplasmic reticulum (ER) by means of addition of signal peptide/leader sequence onto the N-terminus of such an engineered protein, or a fragment of said proteins. Preferably, the protein capable of translocating into an ER has an amino acid sequence which initiates transport of a protein (e.g., the IL-18 or its fragment, variant, or a fragment of its variant) across the membrane of the endoplasmic reticulum.

In some embodiments, suitable proteins capable of translocating into the ER or a fragment thereof of the fusion protein is a globular protein, immunoglobular protein, or a fragment thereof.

In some embodiments, suitable proteins capable of translocating into the ER or a fragment thereof of the fusion protein is a short polypeptide or protein being about 2 kDa or no greater than 250 kDa. As examples, the short polypeptide or protein is about 2-5 kDa, about 6-10 kDa, about 11-20 kDa, about 21-30 kDa, about 31-40 kDa, about 41-50 kDa, about 51-75 kDa, about 76-100 kDa, about 101-125 kDa, about 126-150 kDa, about 151-175 kDa, about 176-200 kDa, about 201-225 kDa, or about 256-250 kDa. As further examples, the short polypeptide is 2, 3, or 4 amino acids, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids. Additional examples of these short polypeptide include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320)), wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein XX is Q, A, E or S and n=1-5 or in some embodiments, an integer larger than 5. Still additional examples include (X_{λ}GG)ₙ wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5.

In various embodiments, the fusion protein comprises further an amino acid linker. For example, the amino acid linker can be between (a) the protein capable of translocating into an endoplasmic reticulum (ER) and (b) the propeptide (or variant) or IL-18 (or variant). As further examples, the short polypeptide is 2, 3, or 4 amino acids, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids. Additional examples of these short polypeptide include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5. (GX_{λ}GG)ₙ wherein XX is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG)ₙ wherein XX is Q, A, E or S and n=1-5 or in some embodiments, an integer larger than 5, (GvX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5. (GX_{λ})ₙ wherein X_{λ} is Q, A, E or S and n=1-5 or in some embodiments, an integer larger than 5.

In various embodiments, the fusion protein comprises further an amino acid linker or amino acid chain on the N terminus of the Il-18 variant. In various embodiments, the fusion protein comprises further an amino acid linker or amino acid chain on the C terminus of the Il-18 variant. Examples of an amino acid linker or amino acid chain (e.g., short polypeptide, short peptide) include but are not limited to 2, 3, or 4 amino acids, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids. Additional examples of these short polypeptide include but are not limited to a dimer of two amino acids, a tri-mer of three amino acids, a 4-mer of four amino acids, a 5-mer of five amino acids, or a peptide selected from the group consisting of T, PT, MPT, S. GS. GGS. GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))n wherein X_{λ} is Q, A, E or S and n=1-5 or in some embodiments, an integer larger than 5, (GGX_{λ}GG (SEQ ID NO:319))ₙ wherein X_{λ} is Q, A, E or Sand n=1-5 or in some embodiments, an integer larger than 5, (GGGX_{λ}G (SEQ ID NO:320))ₙ wherein X is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein XX is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG)ₙ wherein XX is Q, A, E or S and n=1-5 or in some embodiments, an integer larger than 5, (GvX_{λ}G)ₙ wherein X_{λ} is Q, A, E or S and n=1-5 or in some embodiments, an integer larger than 5. (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5.

In some embodiments, the one or more fusion proteins do not comprise an IL-18 propeptide or its variant. An “IL-18 propeptide”, or “propeptide” or “PP” in this invention, may also be used interchangeably, which describes an amino acid sequence linked to IL-18 or IL-18 variant in an IL-18 precursor or IL-18 variant precursor, and which upon removal renders a mature IL-18 or its fragment, mature IL-18 variant or its fragment thereof. For example, an IL-18 propeptide may have a sequence of amino acid residues 1-36 of Uniprot ID Q14116.

In some embodiments, the one or more fusion proteins also include a propeptide (PP) or its variant. Examples of propeptide variant are provided herein, including those in Table 1B. This can inactivate IL-18 or IL-18 variant, and so a propeptide directly or indirectly is linked to the IL-18 or IL-18 variant forms a precursor IL-18 or precursor IL-18 variant. Preferably, the PP or its variant is on the N-terminus end relative to IL-18 (or its fragment, variant, or a fragment of its variant) in the fusion protein. In some embodiments, the one or more fusion proteins also include a cleavage site, which is preferably based on a peptide substrate sensitive to enzymatic/protease cleavage. The cleavage site may be positioned within the PP, between the PP or its variant (if present) and the IL-18 (or its fragment, variant, or a fragment of its variant); or may be positioned between the protein capable of translocating into an ER and the PP (if present); or may be positioned between the protein capable of translocating into an ER and the IL-18 or its fragment, variant, or a fragment of its variant, especially in the absence of a PP. In some embodiments, when PP is present, the cleavage site is positioned within the PP. In further embodiments, the one or more fusion proteins include (i) an IL-18, a fragment of IL-18, an IL-18 variant, or a fragment of the IL-18 variant, (ii), a propeptide (PP) or its variant, which inactivates IL-18, and a cleavage site.

Examples of propeptide variants include a polypeptide having AAEPVEDNX₁INFVAMKFIDNTLYFIAEDDEN, wherein X₁ is any amino acid except cysteine (SEQ ID NO:238). In various embodiments, X₁ is alanine, valine, isoleucine, leucin, methionine, phenylalanine, tyrosine or tryptophan (SEQ ID NO:239). In various embodiments, X₁ is valine (SEQ ID NO:78). In various embodiments, X₁ is serine, threonine, asparagine, or glutamine (SEQ ID NO:240). In various embodiments, X₁ is serine (SEQ ID NO:76).

In various embodiments, the fusion protein does not comprise a polypeptide consisting of the sequence X₁-X₂-X₃-X₄ between (i) the propeptide or propeptide variant, and (ii) the mature IL-18 or mature IL-18 variant, wherein X₁ is L or absent, X₂ is E or absent, X₃ is S or absent, and X₄ is D or absent, when X₁, X₂, X₃, and X₄ are all present the sequence being LESD (SEQ ID NO:253).

In various aspects of the fusion proteins, the IL-18 (or its fragment, variant, or fragment of its variant) is linked by a polypeptide bond to the first protein capable of translocating into the ER. The fusion proteins may have a variety of configurations. Preferably, the N-terminus of the IL-18 (or its fragment, variant, or a fragment of its variant) is linked by a polypeptide bond directly or indirectly to the C-terminus of the first protein capable of translocating in the ER.

Yet in other embodiments, the C-terminus of the IL-18 (or its fragment, variant, or a fragment of its variant) is linked by a polypeptide bond directly or indirectly to the N-terminus of the first protein capable of translocating in the ER. As a nonlimiting example, the IL-18-variant (or IL-18, a fragment of IL-18, a fragment of the IL-18 variant) is fused to the N-terminus of a knob of a knob-into-hole heterodimeric IgG1 protein with or without a propeptide (pp).

In further embodiments, where the C-terminus of the IL-18 is linked to the N-terminus of the first protein capable of translocating in (or through) the ER, a second protein capable of translocating through the ER is often fused to the N-terminus of the IL-18 to mediate masking. It is contemplated that a fusion protein further comprises (iii) a second protein capable of translocating into/through an ER, or a “scaffold” such as heat shock proteins (HSPs) that may not translocate through the ER. In some embodiments, if HSP (nuclear protein) or a cytosolic protein is fused to the N-terminus of the IL-18 to mediate masking, it often requires a signal peptide fused to the N-terminus of the “scaffold” to mediate transport to the ER and, if the scaffold is fused to the C-terminus of IL-18 to serve to stabilize the complex, then a second protein capable of translocating through the ER is often fused to the N-terminus of the IL-18 to mediate masking. Hence, in some embodiments, the IL-18 (or its fragment, variant, or a fragment of its variant) is at the C-terminus of the fusion protein: in some embodiments, the N-terminus of the IL-18 (or its fragment, variant, or a fragment of its variant) is on the C-terminus end relative to the first protein capable of translocating in the ER, and the C-terminus of the IL-18 (or its fragment, variant, or a fragment of its variant) is on the N-terminus end relative to the second protein capable of translocating in the ER. The “first” or “second” protein capable of translocating an ER is used as a relative reference.

One or more exemplary amino acid sequences of each component of the fusion protein are shown in Tables 1 and 4.

Some embodiments provide that the first protein/polypeptide capable of translocating into an ER comprises an immunoglobulin heavy chain constant region. In some embodiments, the immunoglobulin heavy chain constant region comprises an immunoglobulin heavy chain constant region domain selected from the group consisting of a CH2 domain, a CH3 domain, and a CH4 domain, or a combination thereof. In some embodiments, wherein the immunoglobulin heavy chain constant region comprises a CH2 domain and a CH3 domain. In some embodiments, the immunoglobulin heavy chain constant region lacks at least a CH1 domain. In some embodiments, the immunoglobulin heavy chain constant region is a human immunoglobulin heavy chain constant region. In some embodiments, the immunoglobulin heavy chain constant region is an immunoglobulin heavy chain constant region present in the same species as the IL-18. In other embodiments, the immunoglobulin heavy chain constant region is an immunoglobulin heavy chain constant region present in the same species as an organism with which a nucleic acid molecule encoding the fusion protein or a precursor of the fusion protein is transformed or transfected. Further embodiments provide that the fusion protein lacks an immunoglobulin variable domain (V_{H}).

In various embodiments, the IL-18 (or its fragment, variant, or fragment of its variant) is identical (in sequence) to that of a human origin, and the immunoglobulin heavy chain constant region comprises a hinge region, and a CH2 domain or a CH3 domain. In various embodiments, comprises a hinge region and both a CH2 domain and a CH3 domain. More preferably, the IL-18 (or its fragment, variant, or fragment of its variant) is at least 95%, 90%, or 85% identical (in sequence) to that of a human origin, but with amino acid substitutions or other modifications that reduces affinity of the IL-18 (or its fragment, variant, or fragment of its variant) for IL-18BP. It is contemplated that immunoglobulin heavy chain constant regions suitable for the invention may be derived from immunoglobulins belonging to any of the five immunoglobulin classes referred to in the art as IgA (Igα), IgD (Igδ), IgE (Igε), IgG (Igγ), and IgM (Igμ). However, immunoglobulin heavy chain constant regions from the IgG class are preferred. Furthermore, the immunoglobulin heavy chain constant regions may be derived from any of the IgG antibody subclasses referred to in the art as IgG1, IgG2, IgG3, and IgG4. Immunoglobulin heavy chain constant region domains have cross-homology among the immunoglobulin classes. For example, the CH2 domain of IgG is homologous to the CH2 domain of IgA and IgD, and to the CH3 domain of IgM and Igε. Preferred immunoglobulin heavy chain constant regions include protein domains corresponding to a CH2 region and a CH3 region of IgG, or functional portions or derivatives thereof. Further description of immunoglobulin heavy chain constant regions is discussed in detail in U.S. Pat. Nos. 5,541,087, and 5,726,044, which are incorporated by reference herein.

In multiple embodiments, the protein/polypeptide to be fused with the IL-18 (or its fragment, variant, or a fragment of its variant) is a dimer of two immunoglobulin heavy chain constant regions/chains, optionally cross-linked by a pair of disulfide bonds between cysteines on adjacent hinge regions. In some embodiments, a hinge region may have an upper hinge domain, a core hinge domain, and a lower hinge domain. In some embodiments, an upper portion of the hinge domain may include or remove the cysteine that is known to form a disulfide bond with the light chain or a fab, resulting in sequences such as EPKSC (SEQ ID NO:241) or EPKSS (SEQ ID NO:242) or EPKSA (SEQ ID NO:243). For example, fusion proteins including IgG1-based ER translocating protein, except FUSE-501, FUSE-503, and FUSE-509, may have removed cysteine from the hinge region, e.g., EPKSS (SEQ ID NO:242) in IgG1-based ER translocating protein, except for FUSE-507 (FUSE-507 has EPKSA (SEQ ID NO:243) in the hinge region). A hinge region may also contain a core hinge domain, such as comprising a sequence CPPCP (SEQ ID NO:244) or a variant where the cysteine is replaced. A hinge region may further include a lower hinge domain, such as comprising a sequence APELLGGP (SEQ ID NO:245) or APEAAGGP (SEQ ID NO:246). In another example, FUSE-509 has an IgG4-based ER-translocating protein, using a hinge region as depicted in Chiu et al., Antibodies 2019, 8(4), 55, 2019. While constructs including immunoglobulin hinge regions are preferred, as depicted in the drawings, the invention contemplates that crosslinking at other positions may be chosen as desired. Furthermore, in some cases, two or more monomers may associate non-covalently to produce dimers or multimers. In various aspects wherein the protein/polypeptide is a dimer of two immunoglobulin heavy chain constant regions/chains, the IL-18 (or its fragment, variant, or a fragment of its variant) is linked to one, and only one, of the two (or more) immunoglobulin heavy chain constant regions/chains. In the case of a wild type IgG-Fc that forms a homodimer, in various instances, a IL-18 is placed on the C-terminus of each monomer of the Fc, thereby having two IL-18 placed on the C-terminus of the Fc. In some instances, a heterodimer may form (e.g., in purification step) when one wild-type Fc fused to one IL-18 is mixed with another wild-type Fc not fused to IL-18. In other aspects, an IL-18 (or its fragment, variant, or a fragment of its variant) is linked each of the two (or more) immunoglobulin heavy chain constant regions/chains in the fusion protein.

In some embodiments, two arms (or chains) of immunoglobulin heavy chain constant regions (e.g., Fc polypeptides) can be heterodimerized by creating “knobs-in-holes” (KiH) mutations in the CH3 domain. This structural feature in the polypeptide anns allows for assembly of two half antibodies (e.g., Fc heterodimer; and VH-CH and VL-CL domains). For example, a heteromultimer (including a heterodimer) may comprise a first polypeptide and a second polypeptide each comprising a CH3 domain, wherein the polypeptides meet at an engineered interface within the CH3 domain, and the first polypeptide contains an engineered protuberance (“knob”) in the interface with at least one contact residue replaced with an import residue having a larger side chain volume than the original residue, and the second polypeptide contains an engineered cavity (“hole”) in the interface with at least one contact residue replaced with an import residue having a smaller side chain volume than the original residue. In some embodiments, the engineered interface of a heteromultimer includes at least two protuberance-into-cavity mutant pairs. Volumes and accessible surface areas of each amino acid are described in A. A. Zamyatnin, Prog Biophys. Mol. Biol. 24: 107-123, 1972 and C. Chothia, J. Mol. Biol. 105: 1-14, 1975. For example, import residues for the formation of a protuberance can be arginine (R), phenylalanine (F), tyrosine (Y) and tryptophan (W); and preferably the original residue for the formation of the protuberance has a small side chain volume, such as alanine, asparagine, aspartic acid, glycine, serine, threonine or valine. As another example, import residues for the formation of a cavity can be alanine (A), serine (S), threonine (T) and valine (V); and preferably the original residue for the formation of the cavity has a large side chain volume, such as tyrosine, arginine, phenylalanine or tryptophan. For example, a T366W mutation in CH3 domain for the “knob”/protuberance chain, and a T366S/L368A/Y407V mutation in CH3 domain for the “hole”/cavity chain. Additionally, the KiH configuration may be coupled further mutations to permit S—S disulfide linkage between the two chains. In various aspects wherein the protein/polypeptide is a heterodimer of a the KiH configuration, the IL-18 (or its fragment, variant, or a fragment of its variant) is linked to one, and only one, of the two (or more) immunoglobulin heavy chain constant regions/chains (i.e. knob or hole).

In some embodiments, the two or more arms (or chains) of immunoglobulin heavy chain constant regions (e.g., Fc polypeptides) can contain another symmetric-to-asymmetric steric complementarity design (e.g., HA-TF, ZW1), a charge-to-charge swap interaction (DD-KK), a charge-to-steric complementarity swap plus additional long-range electrostatic interaction (e.g., EW-RVT), or an isotype strand swap design (e.g., strand-exchange engineered domain (SEED)), or Xmab, 7.8.60, Electrostatic Steering, A107, or Duobody, so as to form heterodimers/heteromultimers. Further description of these configurations and exemplary mutations/residues are seen in Front Immunol. 2016: 7: 394.

In additional embodiments, suitable proteins capable of translocating into the ER or a fragment thereof of the fusion protein is a globular protein, immunoglobular protein, or a fragment thereof. In various embodiments, suitable proteins capable of translocating into the ER or a fragment thereof of the fusion protein is a short polypeptide or protein engineered with a signal peptide for translocating into the ER. For example, the short polypeptide or protein being about 2 kDa or no greater than 250 kDa. As additional examples, the short polypeptide or protein is about 2-5 kDa, about 6-10 kDa, about 11-20 kDa, about 21-30 kDa, about 31-40 kDa, about 41-50 kDa, about 51-75 kDa, about 76-100 kDa, about 101-125 kDa, about 126-150 kDa, about 151-175 kDa, about 176-200 kDa, about 201-225 kDa, or about 256-250 kDa. As further examples, the short polypeptide is 2, 3, or 4 amino acids, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids. Additional examples of these short polypeptide include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG)ₐ wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5.

In additional embodiments, suitable proteins capable of translocating in an ER can be a globular protein, human serum albumin (HSA), beta2microglobulin, transferrin, fragment antigen-binding region (Fab region), VHH antibody, single-chain variable fragment (scFv), anticalin, designed ankyrin repeat protein (DARPin), a binding domain thereof, and a fragment thereof.

Additional suitable proteins capable of translocating into an ER can include type I transmembrane proteins or a fragment thereof, or type II transmembrane proteins or a fragment thereof.

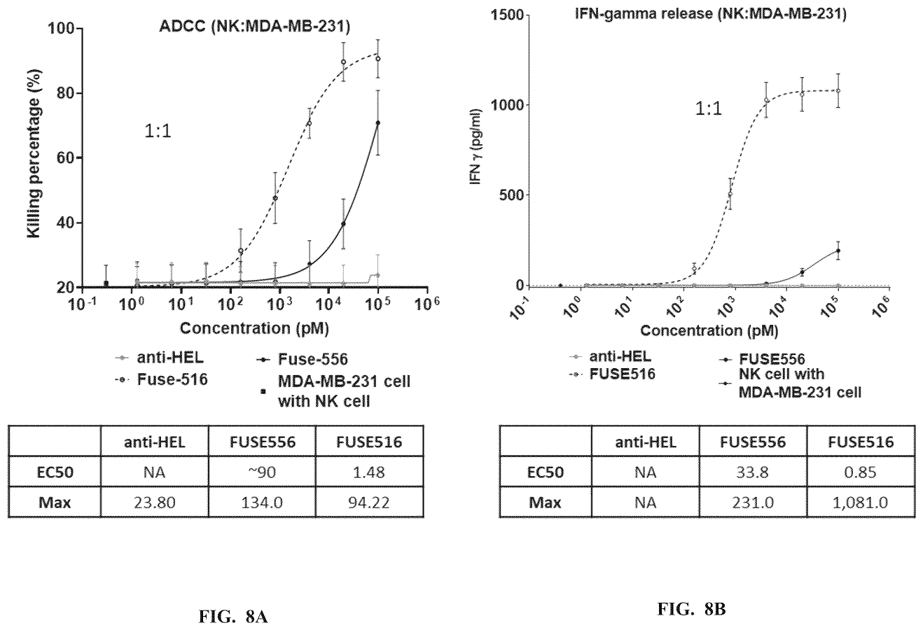

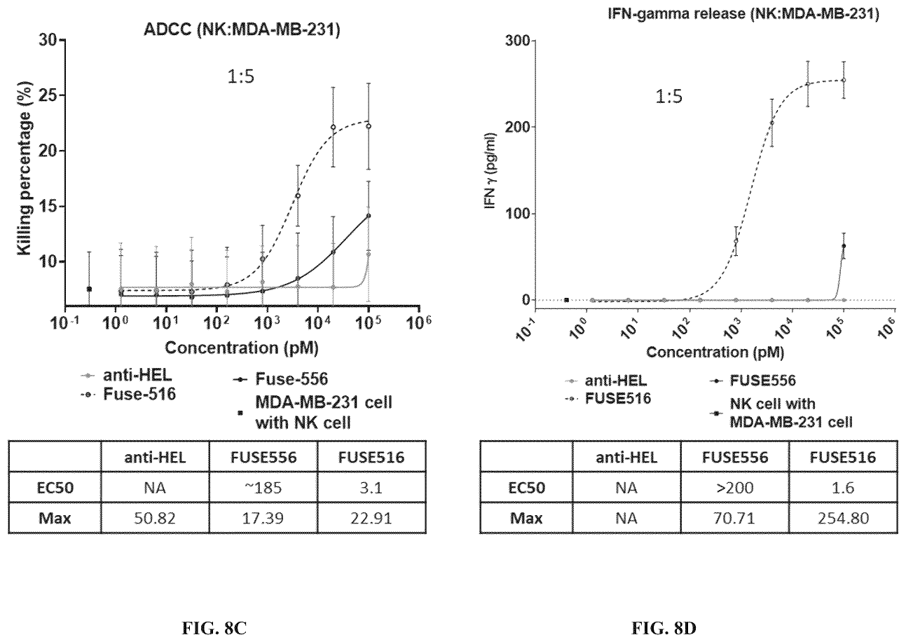

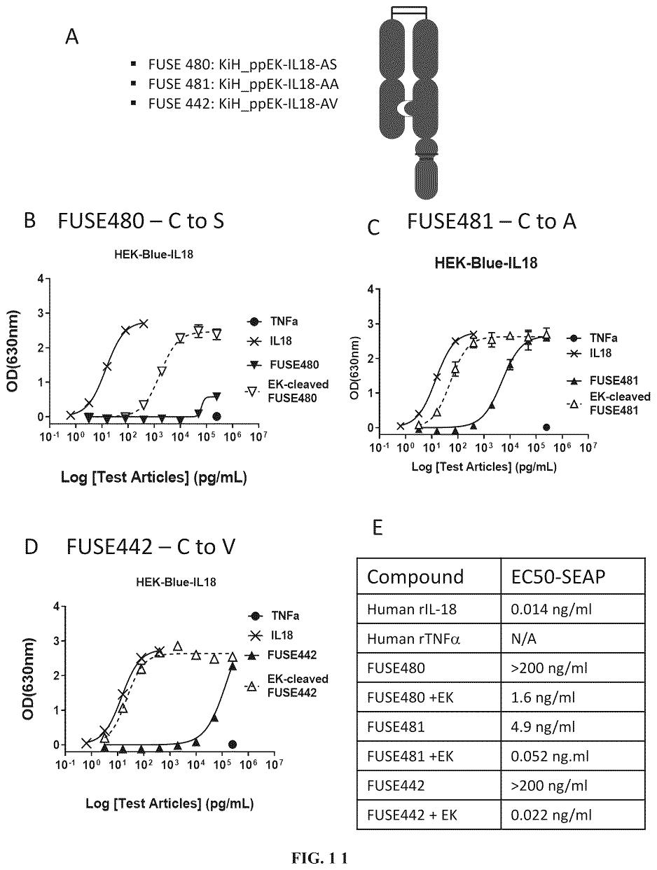

In various embodiments, the fusion protein comprising the short polypeptide or protein and the IL-18 or IL-18 variant, or fragments thereof, further comprises a second proteins capable of translocating into the ER or a fragment thereof. The second protein capable of translocating into the ER or a fragment thereof can be an Fc domain or HSA, beta2microglobulin, transferrin, fragment antigen-binding region (Fab region), VHH antibody, single-chain variable fragment (scFv), anticalin, designed ankyrin repeat protein (DARPin), a binding domain thereof, and a fragment thereof, or type I transmembrane proteins or a fragment thereof, or type II transmembrane proteins or a fragment thereof as described herein. FIGS. 8A and 11A (first three from left to right) are nonlimiting examples of such fusion proteins.

In yet other embodiments, the fusion protein further comprises a protein that cannot naturally translocate into the ER such as nuclear or cytosolic proteins fused to the N-terminus of IL-18. For said proteins, a signal peptide (which may be termed a leader sequence), such as Ig-kappa leader sequence (e.g., METDTLLLWVLLLWVPGSTG (SEQ ID NO:247)) in FUSE-499, or one or more other signal peptides including but not limited to those derived from human albumin and human azurocidin, see Kober et al. Biotechnol Bioeng. 2013 April; 110(4):1164-73 is fused to the N-terminus of the non ER translocating protein. For example, the signal peptide may be on the N-terminus end of the propeptide or of the IL-18 (or its fragment, variant, or a variant of its fragment). A further example of a protein capable of translocating in/into/through ER may be a protein engineered with a signal peptide, e.g., on the N-terminus end. As an example, Hsp70 is a nuclear protein, but can be engineered to be an ER-translocating protein when fused or linked with a signal peptide on the N-terminus of Hsp70. In various embodiments, the addition of an N-terminal signal peptide, such as the Ig-kappa leader sequence, is in place of a Fc, globular protein, or HSS that'd otherwise be present in a fusion protein disclosed herein.

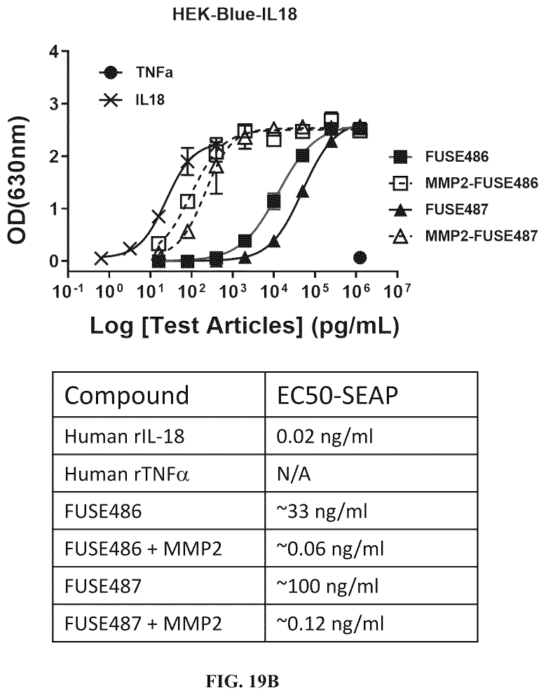

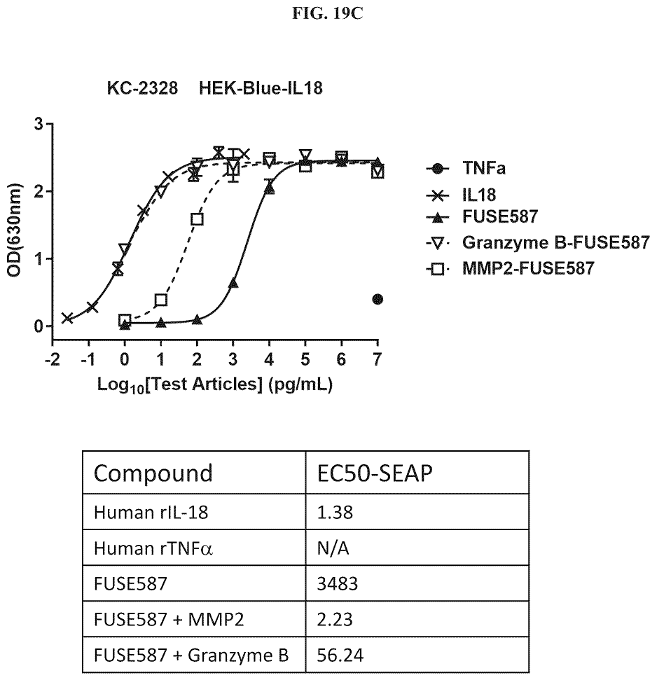

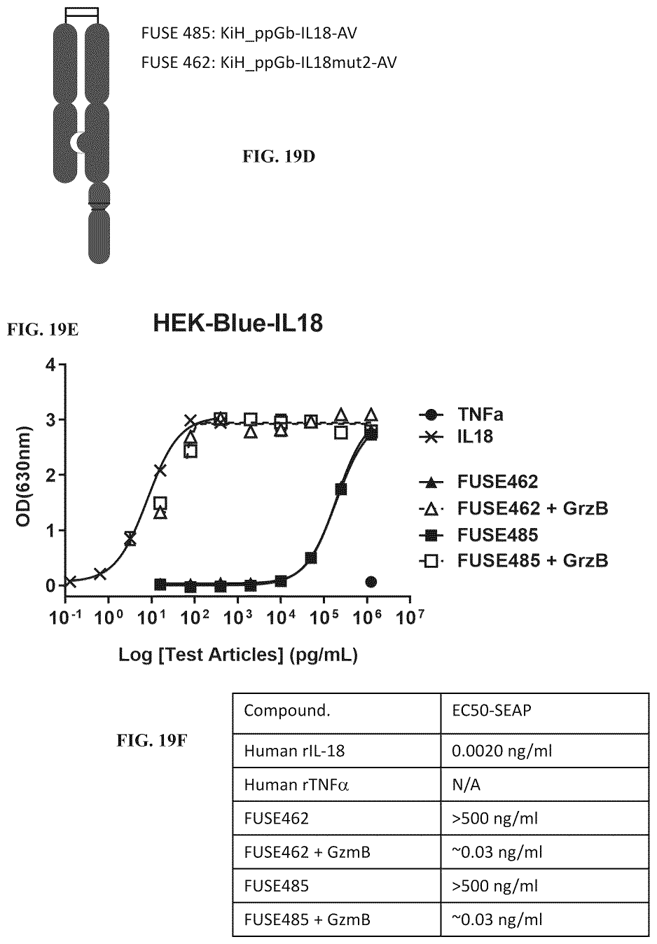

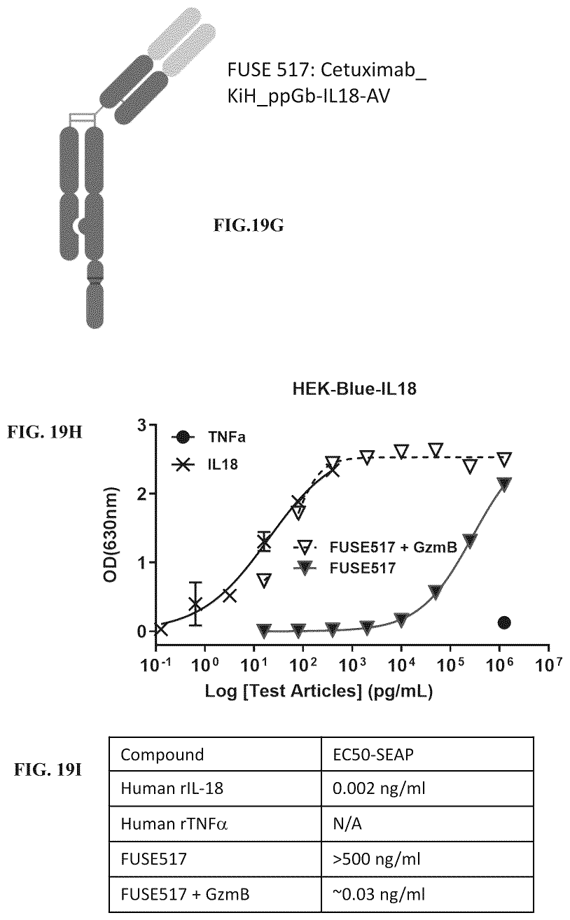

In some embodiments, the fusion protein (e.g., having a masked or unmasked IL-18 variant) further comprises a tumor targeting fragment, e.g., a fragment that targets cell surface proteins including but not limited to a tumor associated antigen (TAA). For example, FUSE-517 as shown in FIG. 19G is a masked IL-18 fusion protein that also comprises an anti-EGFR antibody fragment, e.g., Fab of cetuximab. One or more antigen-targeting (preferably tumor antigen-targeting) fragments of known antibodies are conceived to be compatible with the fusion protein system disclosed herein.

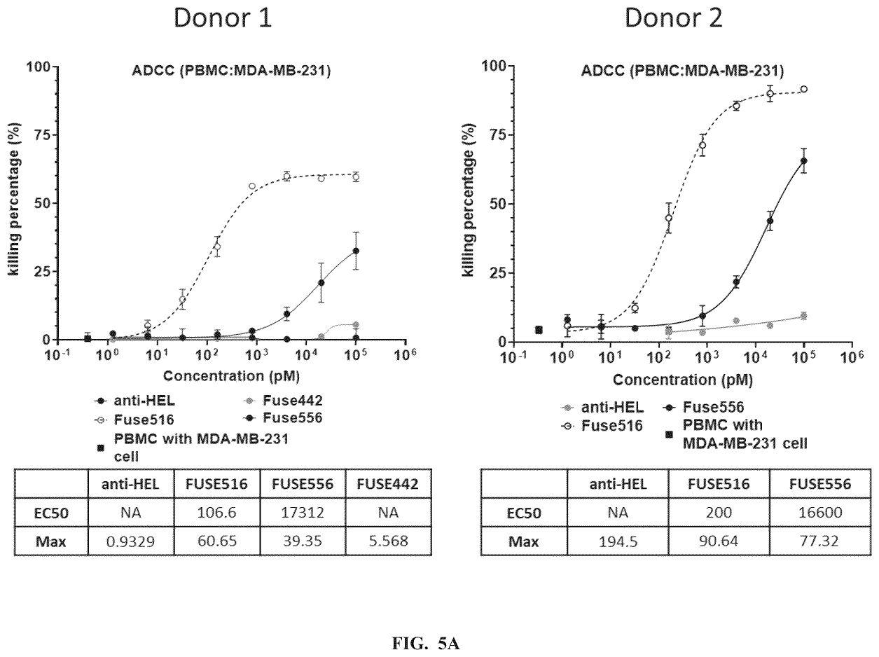

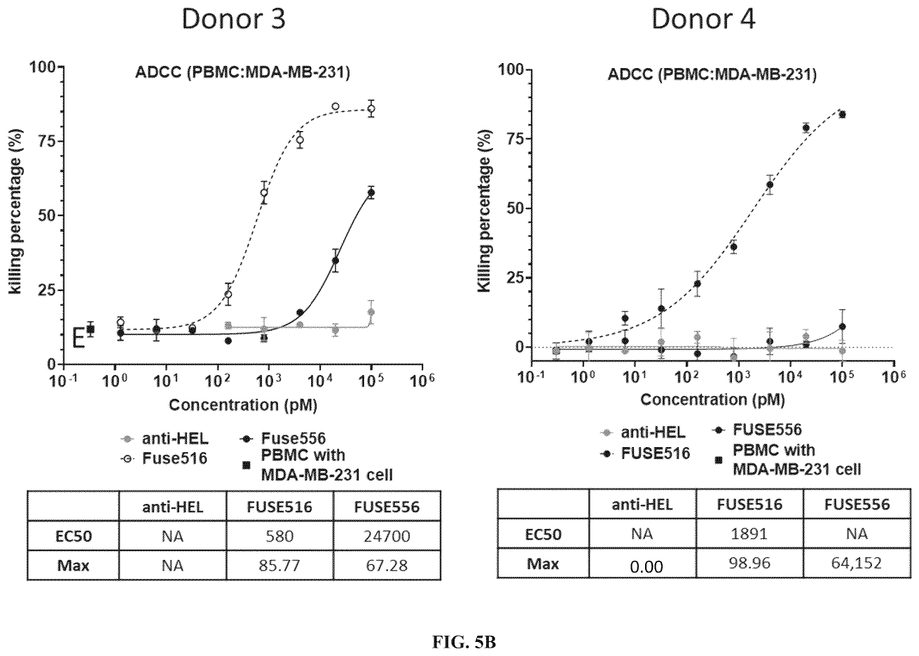

In some embodiments, the fusion protein (e.g., having a masked or unmasked IL-18 variant) comprises an activation receptor targeting fragment, e.g., a fragment that targets activation receptors on cell surface including but not limited to CD16 on natural killer cell surface. Activation receptors include immunoreceptor tyrosine-based activation motif (ITAM)-associated receptors, such as CD16 and NKp46. Activation receptors also include those participating in spontaneous NK cell activation, such as NKp46 (CD335), NKp30 (CD337), NKp44 (CD336), NKG2D (CD314), DNAM-1 (CD226), 2B4 (CD244), LFA-1 (CD11a-CD18), and CD2. In some embodiments, the fusion protein (e.g., a masked IL-18) comprises both an activation receptor targeting fragment and a tumor targeting fragment. For example, FUSE-516 as shown in FIGS. 5A, 5B, 6A, 6B, 7A, 7B, 8A and 8B is a masked IL-18 fusion protein that also comprises an anti-CD16 fragment and an anti-EGFR antibody fragment, e.g., Fab of cetuximab. Hence, FUSE-516 targets both CD16 and EGFR simultaneously. Examples of anti-CD16 fragments include but are not limited to CH2 domains of IgG1, CH2 domain of IgG4.

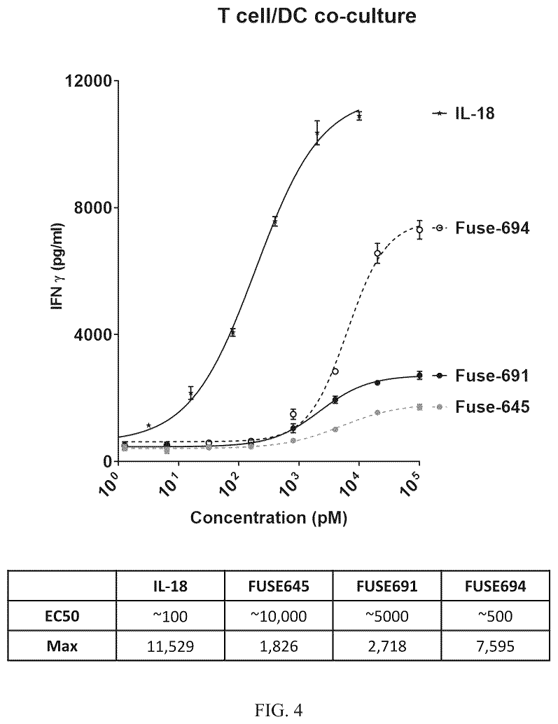

In some embodiments, the fusion protein (e.g., having a masked or unmasked IL-18 variant) comprises a polypeptide fragment that targets an immune checkpoint, e.g., fragment that targets an immune checkpoint expressed on T cell. For example, FUSE-694 as shown in FIG. 4 is a masked IL-18 fusion protein that also comprises an anti-PD1 fragment. Example immune checkpoints include but are not limited to PD-1, PD-L1, CTLA-4, LAG-3. One or more immune checkpoint-targeting fragments of known antibodies are conceived to be compatible with the fusion protein system disclosed herein. Examples of anti-PD1 fragments include fragments (e.g., Fab, Fv) from pembrolizumab, nivolumab, pidilizumab. AMP-514, spartalizumab, cemiplimab. AK105, BCD-100. BI 754091. JS001, LZM009, MGA012, Sym021, TSR-042, MGD013, AK104, XmAb20717, tislelizumab, or PF-06801591. Additional examples of anti-PD1 fragments also include fragment (e.g., Fab, Fv) from vopratelimab, camrelizumab, sintilimab, AMP-224, AMP-514, and Acrixolimab. In various embodiments, the antibody is an anti-PD-1 antibody or anti-PD-L1 antibody. Examples of anti-PD1 fragments include fragment (e.g., Fab, Fv) from anti-PD1 antibodies such as pembrolizumab, nivolumab, pidilizumab, AMP-224, AMP-514, spartalizumab, cemiplimab, penpulimab (AK105), prolgolimab (BCD-100), ezabenlimab (BI 754091), toripalimab (JS001), lipustobart (LZM009), retifanlinab (MGA012), Sym021, dostarlimab (TSR-042), tebotelimab (MGD013), cadonilimab (AK104), vudalimab (XmAb20717), tislelizumab, PF-06801591, and anti-PD1 antibody expressing pluripotent killer T lymphocytes (PIK-PD-1). Examples of anti-PD-L1 fragments include fragment (e.g., Fab, Fv) from anti-PD-L1 antibodies such as garivulimab (BGB-A333), cosibelimab (CK-301), FAZ053, envafolimab (KN035), MDX-1105, betifisolimab (MSB2311), adebrelimab (SHR-1316), atezolizumab, avelumab, durvalumab. BMS-936559, CK-301, and M7824. Thus, for example, the fusion protein comprises an anti-PD1 fragment from an anti-PD1 antibody (e.g., Fab, Fv), an IL-18 variant, and a protein capable of translocating into an ER (e.g., a Fc domain), and optionally, a linker.

In some embodiments, the fusion protein (e.g., having a masked or unmasked IL-18 variant) comprises a targeting polypeptide, wherein the targeting polypeptide targets a protein on the same surface as the IL-18 RC. Examples of such proteins include but are not limited to CD16, γ9 TCR, δ2 TCR or δ1 TCR, NKp46, CD137. CD40 or NKG2D. In some embodiments, fusion protein (e.g., having a masked or unmasked IL-18 variant) comprises a targeting polypeptide, wherein the targeting polypeptide targets a protein on a cell that does not contain an IL-18RC. In these embodiments, the IL-18 fusion protein will need to be delivered close to the IL-18R complex for a cis or density effect to result in the interaction between the IL-18 fusion protein and the IL-18R complex. For example, TAA targeted IL-18 fusion protein could get to interact with the IL-18R complex on T cells if (a) the fusion protein bridged T cells with TAA+ cells or (b) the fusion protein was combined with another protein that bridged T cells with TAA+ cells or (c) the fusion protein bound to a TAA+ cell that naturally interacted with T cell via a secondary means (e.g. TCR/MHC interaction). In other examples, the fusion protein could be delivered to fibroblasts or other accessory cells in the tumor microenvironment and released by proteases such that it can act on IL-18R+ T or NK cells at a distance.

Exemplary targeting polypeptides include those noted in Table 5 or fragments thereof. Of those listed as the antigen-binding antibodies, their VHH, Fab regions, or single-chain variable fragments (scFv) can be used as the antigen-binding site of the multispecific antibodies disclosed herein.

In some embodiments, enterokinase is used for site-specific cleavage of recombinant fusion proteins containing an accessible enterokinase recognition site. For example, enterokinase can specifically cleave after the C-terminal end of the lysine residue at its cleavage site, Asp-Asp-Asp-Asp-Lys (SEQ ID NO:87). Therefore, the fragment produced from this cleavage reaction does not inherit any residues from the DDDDK (SEQ ID NO:87) recognition sequence. Additionally. DDDDK (SEQ ID NO:87) is a part of the octapeptide FLAG tag (DYKDDDDK (SEQ ID NO:248)), which can be utilized as a fusion tag for recognition by antibody, and for detection of fusion protein with Western blot analysis, as well as for purification of the fusion protein by Anti-FLAG affinity chromatography.

Preferably, a cleavage site can be based on peptide substrates sensitive to other enzymes, especially proteases highly expressed in tumor microenviromnent, such as granzyme B, granzyme A, granzyme M, granzyme K, matrix metalloproteinase (MMP) 1/2/9/14 or other MMPs. Of note, granzymes are usually only upregulated in inflamed tumors. For example, a substrate sequence for granzyme B can be Ile-Glu-Xaa-Asp↓Xaa-Gly (SEQ ID NO:249) with the cleavage at the Asp↓Xaa peptide bond. Alternatively, a substrate sequence for granzyme B can also be Ile-Glu-Xaa-Asp↓, with the cleavage at the C-terminus end of Asp, and Xaa can be Gln (SEQ ID NO:88) or another amino acid.

In additional embodiments, the fusion protein comprises a cleavage site recognized by a serine protease, a cysteine protease, an aspartate protease, a threonine protease, a glutamic acidprotease, a metalloproteinase, a gelatinase, or an asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, ahK1, a hK10, a hK15, a plasmin, a collagenase, a Type IV collagenase, a stromelysin, a Factor Xa, a chymotrypsin-like protease, a trypsin-like protease, an elastase-like protease, a subtilisinlike protease, an actinidain, a bromelain, a calpain, a caspase, a caspase-3, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM17, an ADAM12, an urokinase plasminogen activator (uPA), an enterokinase, a prostate-specific target (PSA, hK3), an interleukin-10 converting enzyme, a thrombin, a FAP (FAP-α), a dipeptidyl peptidase, or dipeptidyl peptidase IV (DPPIV/CD26), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26). Nonlimiting examples of cleavage sites are included in Table 1B. As a particular example, IEQD (SEQ ID NO:88) can be used.

It is contemplated that a variant, fragment, or a fragment of a variant of the IL-18 is suitable, and in some embodiments preferred, for the composition of the fusion protein. For example, a variant of mature IL-18 can have one, two, three, four, five, or more amino acid substitutions compared to the wild type mature IL-18. For example, one or more cysteines in the IL-18 or its propeptide are replaced with a natural or non-natural amino acid (other than cysteine), such as from Cys to Ser, Ala, or Val, so as to reduce aggregation of the molecule in the fusion protein. Additional examples include cysteine to threonine, asparagine, or glutamine; cysteine to alanine, isoleucine, leucine methionine, phenylalanine, tyrosine, or tryptophan; cysteine to phenylalanine, alanine, aspartic acid, or asparagine; or cysteine to threonine, glutamine, aspartic acid, phenylalanine, isoleucine or histidine.

A variant of IL-18 may have a 95%, 90%, 85%, 83%, 80%, 75%, 70%, 65% or at least 60% sequence identity to wild type IL-18. In some embodiments, a variant of IL-18 may have at least 60% and at most 83% sequence identity to wild type IL-18. In some embodiments, a variant of IL-18 may have between 60%-64%, 65%-69%, 70%-74%, 75%-89%, or 80%-83% sequence identity to wild type IL-18. In some embodiments, the variant of IL-18 in the fusion protein is released as an about 15 kDa functional fragment (e.g., on the electrophoresis gels as tested) when cleaved at a cleavage site of the fusion protein. (It is conceived that the released protein may be mature IL-18 which would normally run at 18 kDa but may appear as about 15 kDa due to the ladder being used or the specific polyacrylamide percentage in a gel.) A fragment of IL-18 may have a 95%, 90%, 85%. 83%, 80%, 75%, 70%, 65%, or at least 60% sequence identity (and/or length) to its corresponding wild type IL-18 fragment. In some embodiments, an IL-18 fragment produced by the fusion protein disclosed herein, especially after protease cleavage of the fusion protein, is less than 85% (e.g., about 83%, about 83%-80%, about 80%-75%, about 75%-70%, or about 70%-65%) in size compared to natural/wild-type mature IL-18; for example, an IL-18 fragment of about 15 kDa in size, preferably having comparable binding affinity for IL-18Ra/b as the wild type mature IL-18, is fused to a propeptide (or PP variant) and an ER translocating protein (with or without mutations), and the fusion protein also includes a protease cleavage site, such that upon protease cleavage, a small IL-18 fragment (e.g., about 15 kDa in size), is released. Preferably, this small IL-18 fragment maintains the natural binding affinity for IL-18Ra/b and an equal or lower binding affinity relative to IL-18Ra/b for IL-18BP. Preferably a variant, fragment, or a fragment of a variant of the IL-18 is capable of binding IL-18R and forming complex, so as to activate proinflammatory programs and/or NF-κB pathway. In some embodiments, the variant, fragment, or a fragment of a variant of the IL-18 is capable of having an increased binding affinity (e.g., 150%, 140%. 130%, 120%, 110%, or at least 100% relative to wild type IL-18) and/or inducing the biological activity of at least 150%, 140%, 130%, 120%, 110%, 100%, 90%, 80%, 70%, 60%, 50%, 40%, 30%, 20%, or 10%, compared to wild type IL-18. In some embodiments, the variant, fragment, or a fragment of a variant of the IL-18 is capable of having an increased binding affinity at 120%, 110%, or at least 100% relative to wild type IL-18, and/or inducing the biological activity of at 120%, 110%, 100%, 90%, 80%, 70%, 60%, 50%, 40%, 30%, 20%, or 10%, compared to wild type IL-18. In additional embodiments, a variant, fragment, or a fragment of a variant of the IL-18 has diminished binding to IL-18 binding protein (IL-18BP), as compared to a wild type IL-18

In some embodiments, the IL-18 or its fragment or variant cleaved from the fusion protein has at least 1,000, 2,000, 3,000, 5,000, 10,000, 30,000, 50,000, 70,000, 80,000, 90,000, or 100,000-fold increase in biological activity (e.g., binding with IL-18R to form IL-18/IL-18Rα/β complex and induce downstream signaling), compared to an uncleaved form in the fusion protein especially with propeptide. In further embodiments, the IL-18 or its fragment or variant cleaved from the fusion protein has a comparable biological activity, or within about 10, 20, 30, 40, or 50-fold difference in the biological activity, compared to recombinant human mature IL-18.

In some embodiments, the IL-18 or its fragment or variant cleaved from the fusion protein has a binding affinity for its IL-18R complex with an equilibrium dissociation constant (KD) of about 18 nM, (e.g., 18 nM±0.3 nM, 18 nM±0.5 nM, 18 nM±1.0 nM). In some embodiments, the IL-18 or its fragment or variant cleaved from the fusion protein has a binding affinity for its IL-18R complex which is about the same, or at least 100%, 95%, or 90%, compared to that of the wild type IL-18. In some embodiments, the IL-18 or its fragment or variant cleaved from the fusion protein has a binding affinity for its IL-18R complex which is greater than that of the wild type IL-18, e.g., a binding affinity that is at least 105%, 110% compared to that of the wild type IL-18, or having a KD value at least 10% or 20% smaller than that of wild type IL-18. Preferably, the IL-18 or its fragment or variant cleaved from the fusion protein has a reduced binding affinity for IL-18BP, compared to that of the wild type IL-18. For example, in some instances, the IL-18 or its fragment or variant cleaved from the fusion protein has a KD with IL-18BP of 18 nM or greater, such that it has a lower binding affinity to IL-18BP than to IL-18R. In some instances, the IL-18 or its fragment or variant cleaved from the fusion protein has a KD with IL-18BP of 18 nM or greater, whereas the wild type IL-18 has a KD with IL-18BP of about 0.4 nM. It is also conceived that KD may vary depending on instrument and protocol setup.

In accordance with the methods described herein, polynucleotides encoding the fusion proteins are also disclosed herein. For example, the nucleic acid sequences may encode in a 5′ to 3′ direction, a first protein or polypeptide capable of translocating in an ER and an IL-18 (or its fragment, variant, or a fragment of its variant). Nonlimiting examples of such polynucleotides are in Table 2.

Furthermore, the polynucleotides optionally may also include a “leader” or “signal” sequence based upon, for example, (1) a propeptide (PP) linked directly to IL-18 (or IL-18 variant) as in FUSE499 or (2) an immunoglobulin light chain sequence fused directly to a hinge region of the immunoglobulin heavy chain constant region. In some embodiments, when the protein/polypeptide capable of translocating in an ER is based upon IgG sequences, the nucleic acid encodes in a 5′ to 3′ direction, at least an immunoglobulin hinge region (i.e., a hinge region containing at least one cysteine amino acid capable of forming a disulfide bond with a second immunoglobulin hinge region sequence), an immunoglobulin CH2 domain and a CH3 domain, and an IL-18 (or its fragment, variant, or a fragment of its variant).

In various embodiments, a polynucleotide encoding the fusion proteins may also be integrated within a replicable expression vector. Hence, a vector encoding the fusion protein is also provided, which may express the fusion protein in, for example, a bacterial host, an intended recipient, or both.

Additional embodiments provide cells transformed or transfected with one or more nucleic acid molecules (polynucleotide) encoding the fusion protein. The cell can be a prokaryotic cell. Or the cell is a eukaryotic cell, preferably a mammalian cell, and more preferably a human cell. Examples of mammalian cells include Chinese hamster ovary (CHO) cells, NS0 cells (a mouse myeloma cell line). PER.C6® cells, and human embryonic kidney cells (HEK cells).

In some embodiments, a non-human organism transformed or transfected with one or more nucleic acid molecules encoding the fusion protein is also provided.

Further embodiments provide a composition comprising combinations of two or more different fusion proteins, or combinations of the nucleic acid sequences encoding the fusion proteins. For example, a pharmaceutical composition is provided, wherein the fusion protein or a nucleic acid molecule encoding the fusion protein is an active agent.

### Methods of Producing Fusion Proteins

Methods for making the fusion proteins, or nucleic acids encoding the fusion proteins for use in the inventive methods are also described herein. Some embodiments provide that conventional recombinant DNA methodologies are utilized for generating the fusion proteins. The fusion constructs preferably are generated at the DNA level, and the resulting DNAs integrated into expression vectors, and expressed to produce the fusion proteins of the invention. Subsequently, the vector is expressed in a host cell to obtain the fusion protein; and optionally the method further includes a step of recovering the fusion protein from the host cell culture. In some embodiments, a method of producing interleukin 18 (IL-18), a fragment thereof, an IL-18 variant, or a fragment of the IL-18 variant, comprises culturing a cell transfected with an expression vector comprising a nucleic acid encoding a fusion protein, in cell culture medium to allow a fusion protein to be produced and secreted into the extracellular space for purification; said protein, when contacting a protease to the fusion protein to cleave the fusion protein to produce the IL-18, the fragment thereof, the IL-18 variant, or the fragment of the IL-18 variant. Exemplary nucleic acid molecules encoding a fusion protein are seen in Table 2. Other embodiments provide that chemical conjugation using conventional chemical cross-linkers may be used to fuse protein moieties.

In some embodiments, the nucleic acid molecules encoding the fusion proteins is expressed in CHO cells or HEK-293. Preferably, expressing the fusion proteins in the host cells results in a recoverable secreted fusion protein of at least 135 mg/L from supernatant of the host cells. In some embodiments, a yield of the fusion protein of at least 135 mg/L is obtained via transient transfection. In some embodiments, an even higher yield of the fusion protein, e.g., at least 150, 200, 250, or 300 mg/L is obtained via stable producer cell clones, or pools of clones. In some embodiments, from a transient transfection, a yield of the fusion protein is about 130-400 mg/L. In some embodiments, a fusion protein, or the IL-18, the fragment thereof, the IL-18 variant, or the fragment of the IL-18 variant cleaved from the fusion protein, is recovered in more than about 400 mg/L, between 350-400 mg/L, 300-350 mg/L, 200-300 mg/L, 100-200 mg/L, or at least 50 mg/L from supernatant of the host cells.

In some embodiments, using a Chinese hamster ovary (CHO) expression system, the fusion protein is produced via a process including the steps of (1) cell recovery, which may be to recover frozen CHO cells via water bath at 37° C.: (2) cell subculturing, which may be to sub-culture the cells and adjust the cell density to 6×10⁶/ml for transfection; (3) transfection and expression, using a solution 1 (in which a plasmid is diluted with a diluting agent), a solution 2 (in which a transection reagent is diluted with a/the diluting agent), and then mixing the solution 1, the solution 2 and the CHO cells, followed by incubating the mixture at a shaker for expression for 12-14 days at 32° C. to collect the supernatant of the culture after centrifuge.

In some embodiments, a purification process is performed after the expression of the fusion protein. In some embodiments, a purification process includes the steps of: (1) washing a column with a binding buffer (10 times volume) at a flow rate of 1 mL/min; (2) loading a fusion-protein-containing sample into the column at a flow rate of 1 mL/min: (3) washing the column with 10× volumes of PBS buffer with a flow rate of 1 mL/min; (4) eluting the protein from the column with 40 mM sodium citrate (pH3.4); optionally the elution sample may be collected into tubes (1 ml/min) and measured for optical density (OD) using NanoDrop at 280 nm; and (5) performing dialysis, e.g., against PBS buffer in a dialysis bag overnight.

### Fusion Proteins

Various Embodiments provide for fusion proteins as described herein. In particular embodiments, the fusion protein is selected from the group consisting of FUSE-557. FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016. FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177.

Various embodiments provide for fusion proteins as described herein. In particular embodiments, the fusion protein comprises the IL-18 variant from FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016. FUSE-1017, FUSE-1041, FUSE-1042, FUSE-1044, FUSE-1109, FUSE-1110, FUSE-1111. FUSE-1112. FUSE-1113. FUSE 1116, FUSE-1176, or FUSE-1177; and a short polypeptide.

In particular embodiments, the fusion protein comprises a mature IL-18 variant selected from Table 1B; and a short polypeptide.

In various embodiments, the short polypeptide is on the N-terminus end. In other embodiments, the short polypeptide is on the C-terminus end.

In various embodiments, the short polypeptide is 2, 3, or 4 amino acids. In various embodiments, the short polypeptides are flexible linkers or amino acid chain of 2-5, 6-10, 11-15, 16-20, 21-25, 26-30 amino acids.

In various embodiments, the short polypeptide is 1, 2, 3, or 4 amino acids. In various embodiments, the short polypeptides are 5, 6, 7, 8, 9, 10, 11, 12, 13, 14, 15, 16, 17, 18, 19, 20, 25, 30, or 35 amino acids.

Examples of these short polypeptides include but are not limited to a dimer of two amino acids, a tri-mer of three amino acids, a 4-mer of four amino acids, a 5-mer of five amino acids, or a polypeptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q. A, E or S and n=1-5 or in some embodiments, an integer larger than 5, (GX_{λ}GGG (SEQ ID NO:318)), wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG) wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5.

### IL-18 Variants

Various embodiments provide for IL-18 variants described herein. In particular embodiments, the IL-18 variant is the IL-18 variant in FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177. That is, the IL-18 variant is the mature IL-18 variant from Table 1B that corresponds with these designations.

TABLE 1A
Amino acid sequences of respective components in exemplary fusion proteins (“FUSE”). Some of “Polypeptide 1” sequences, i.e., first
polypeptide arm sequences, are identical in FUSE-422, FUSE-423, FUSE-424, FUSE-441, FUSE-442, FUSE-462, FUSE-480, FUSE-481, FUSE-484,
FUSE-485, FUSE-486, FUSE-487, FUSE-499, FUSE-500, FUSE-505, FUSE-516, FUSE-517, FUSE-545, FUSE-546, FUSE547, FUSE5-556, FUSE-583-587,
FUSE-599-602, FUSE-645, FUSE-686, FUSE-756-758, FUSE-775, FUSE-874-876, and FUSE-878-892 . Residues represented by a lower-case letter are
amino acid locations with potential mutations.

| | Polypeptide 1 | Polypeptide 2 | Polypeptide 3 |
|---|---|---|---|
| FUSE-422 | EPKSSDKTHTCPPCPAPELLGGPSVFLFPPKPK | EPKSSDKTHTCPPCPAPELLGGPSVFLFPPKPKDTLMISRTPEVTCVVVDVSHEDPEVK | |
| | DTLMISRTPEVTCVVVDVSHEDPEVKFNWYVDG | FNWYVDGVEVHNAKTKPREEQYNSTYRVVSVLTVLHQDWLNGKEYKCKVSNKALPAPIE | |
| | VEVHNAKTKPREEQYNSTYRVVSVLTVLHQDWL | KTISKAKGQPREPQVYTLPPSREEMTKNQVSLWCLVKGFYPSDIAVEWESNGQPENNYK | |
| | NGKEYKCKVSNKALPAPIEKTISKAKGQPREPQ | TTPPVLDSDGSFFLYSKLTVDKSRWQQGNVFSCSVMHEALHNHYTQKSLSLSPGKAAEP | |
| | VYTLPPSREEMTKNQVSLSCAVKGFYPSDIAVE | VEDNSINFVAMKFIDNTLYFIAEDDENDDDDKYFGKLESKLSVIRNLNDQVLFIDQGNR | |
| | WESNGQPENNYKTTPPVLDSDGSFFLVSKLTVD | PLFEDMTDSDSRDNAPRTIFIISKYGDSQPRGLAVTISVKSEKISTLSSENKIISFKEM | |
| | KSRWQQGNVFSCSVMHEALHNRFTQKSLSLSPG | NPPDNIKDTKSDIIFFQRSVPGHDNKVQFESSSYEGYFLASEKERDLFKLILKKEDELG | |
| | K (SEQ ID NO: 1) | DRSIMFTVQNED (SEQ ID NO: 20) | |
| FUSE-423 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNAINFVAMKFIDNTLYFIAEDDENDDDDKYFGKLESKLSVIRNLNDQVLFIDQGNR | |
| | | PLFEDMTDSDARDNAPRTIFIISKYGDSQPRGLAVTISVKAEKISTLSAENKIISFKEM | |
| | | NPPDNIKDTKSDIIFFQRSVPGHDNKVQFESSSYEGYFLAAEKERDLFKLILKKEDELG | |
| | | DRSIMFTVQNED (SEQ ID NO: 21) | |
| FUSE-424 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENDDDDKYFGKLESKLSVIRNLNDQVLFIDQGNR | |
| | | PLFEDMTDSDVRDNAPRTIFIISKYGDSQPRGLAVTISVKVEKISTLSVENKIISFKEM | |
| | | NPPDNIKDTKSDIIFFQRSVPGHDNKVQFESSSYEGYFLAVEKERDLFKLILKKEDELG | |
| | | DRSIMFTVQNED (SEQ ID NO: 22) | |
| FUSE-441 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKGDDD | |
| | | DKYFGKLESKLSVIRNLNDQVLFIDQGNRPLFEDMTDSDVRDNAPRTIFIISKYGDSQP | |
| | | RGLAVTISVKVEKISTLSVENKIISFKEMNPPDNIKDTKSDIIFFQRSVPGHDNKVQFE | |
| | | SSSYEGYFLAVEKERDLFKLILKKEDELGDRSIMFTVQNED (SEQ ID NO: 23) | |
| FUSE-442 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENDDDDKYFGKLESKLSVIRNLNDQVLFIDQGNR | |
| | | PLFEDMTDSDVRDNAPRTIFIISMYKDSQPRGMAVTISVKVEKISTLSVENKIISFKEM | |
| | | NPPDNIKDTKSDIIFFQRSVPGHDNKMQFESSSYEGYFLAVEKERDLFKLILKKEDELG | |
| | | DRSIMETVQNED (SEQ ID NO: 24) | |
| FUSE-462 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENIEQDYFGKLESKLSVIRNLNDQVLFIDQGNRP | |
| | | LFEDMTDSDVRDNAPRTIFIISKYGDSQPRGLAVTISVKVEKISTLSVENKIISFKEMN | |
| | | PPDNIKDTKSDIIFFQRSVPGHDNKVQFESSSYEGYFLAVEKERDLEKLILKKEDELGD | |
| | | RSIMFTVQNED (SEQ ID NO: 25) | |
| FUSE-480 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNSINFVAMKFIDNTLYFIAEDDENDDDDKYFGKLESKLSVIRNLNDQVLFIDQGNR | |
| | | PLFEDMTDSDSRDNAPRTIFIISMYKDSQPRGMAVTISVKSEKISTLSSENKIISFKEM | |
| | | NPPDNIKDTKSDIIFFQRSVPGHDNKMQFESSSYEGYFLASEKERDLFKLILKKEDELG | |
| | | DRSIMFTVQNED (SEQ ID NO: 26) | |
| FUSE-481 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNAINFVAMKFIDNTLYFIAEDDENDDDDKYFGKLESKLSVIRNLNDQVLFIDQGNR | |
| | | PLFEDMTDSDARDNAPRTIFIISMYKDSQPRGMAVTISVKAEKISTLSAENKIISFKEM | |
| | | NPPDNIKDTKSDIIFFQRSVPGHDNKMQFESSSYEGYFLAAEKERDLFKLILKKEDELG | |
| | | DRSIMFTVQNED (SEQ ID NO: 27) | |
| FUSE-484 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENDDDDKYFGKLESKLSVIRNLNDQVLFIDQGNR | |
| | | PLFEDMTDSDVRDNAPRTIFIISKYGDSGARGLAVTISVKVEKISTLSVENKIISFKEM | |
| | | NPPDNIKDTKSDIIFFERDVPGHSGKVQFESSSYEGYFLAVEKERDLFKLILKKEDELG | |
| | | DRSIMFTVQNED (SEQ ID NO: 28) | |
| FUSE-485 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINEVAMKFIDNTLYFIAEDDENIEQDYFGKLESKLSVIRNLNDQVLFIDQGNRP | |
| | | LFEDMTDSDVRDNAPRTIFIISMYKDSQPRGMAVTISVKVEKISTLSVENKIISFKEMN | |
| | | PPDNIKDTKSDIIFFQRSVPGHDNKMQFESSSYEGYFLAVEKERDLFKLILKKEDELGD | |
| | | RSIMFTVQNED (SEQ ID NO: 29) | |
| FUSE-486 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENGPLGVRGGYFGKLESKLSVIRNLNDQVLFIDQ | |
| | | GNRPLFEDMTDSDVRDNAPRTIFIISMYKDSQPRGMAVTISVKVEKISTLSVENKIISF | |
| | | KEMNPPDNIKDTKSDIIFFQRSVPGHDNKMQFESSSYEGYFLAVEKERDLFKLILKKED | |
| | | ELGDRSIMFTVQNED (SEQ ID NO: 30) | |
| FUSE-487 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENVHMPLGFLGPGGYFGKLESKLSVIRNLNDQVL | |
| | | FIDQGNRPLFEDMTDSDVRDNAPRTIFIISMYKDSQPRGMAVTISVKVEKISTLSVENK | |
| | | IISFKEMNPPDNIKDTKSDIIFFQRSVPGHDNKMQFESSSYEGYFLAVEKERDLFKLIL | |
| | | KKEDELGDRSIMFTVQNED (SEQ ID NO: 31) | |
| FUSE-499 | (SEQ ID NO: 1) | AAEPVEDNVINFVAMKFIDNTLYFIAEDDENLESDYFGKLESKLSVIRNLNDQVLFIDQ | |
| | | GNRPLFEDMTDSDVRDNAPRTIFIISMYKDSQPRGMAVTISVKVEKISTLSVENKIISF | |
| | | KEMNPPDNIKDTKSDIIFFQRSVPGHDNKMQFESSSYEGYFLAVEKERDLFKLILKKED | |
| | | ELGDRSIMFTVQNEDEPKSSDKTHTCPPCPAPELLGGPSVFLFPPKPKDTLMISRTPEV | |
| | | TCVVVDVSHEDPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLWCLVKGFYPSDI | |
| | | AVEWESNGQPENNYKTTPPVLDSDGSFFLYSKLTVDKSRWQQGNVFSCSVMHEALHNHY | |
| | | TQKSLSLSPGK (SEQ ID NO: 32) | |
| FUSE-500 | (SEQ ID NO: 1) | YFGKLESKLSVIRNLNDQVLFIDQGNRPLFEDMTDSDVRDNAPRTIFIISMYKDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 33) | |
| FUSE-501 | DAHKSEVAHRFKDLGEENFKALVLIAFAQYLQQ | | |
| | CPFEDHVKLVNEVTEFAKTCVADESAENCDKSL | | |
| | HTLFGDKLCTVATLRETYGEMADCCAKQEPERN | | |
| | ECFLQHKDDNPNLPRLVRPEVDVMCTAFHDNEE | | |
| | TELKKYLYEIARRHPYFYAPELLFFAKRYKAAF | | |
| | TECCQAADKAACLLPKLDELRDEGKASSAKQRL | | |
| | KCASLQKFGERAFKAWAVARLSQRFPKAEFAEV | | |
| | SKLVTDLTKVHTECCHGDLLECADDRADLAKYI | | |
| | CENQDSISSKLKECCEKPLLEKSHCIAEVENDE | | |
| | MPADLPSLAADEVESKDVCKNYAEAKDVFLGMF | | |
| | LYEYARRHPDYSVVLLLRLAKTYETTLEKCCAA | | |
| | ADPHECYAKVEDEFKPLVEEPQNLIKQNCELFE | | |
| | QLGEYKFQNALLVRYTKKVPQVSTPTLVEVSRN | | |
| | LGKVGSKCCKHPEAKRMPCAEDYLSVVLNQLCV | | |
| | LHEKTPVSDRVTKCCTESLVNRRPCFSALEVDE | | |
| | TYVPKEFNAETFTFHADICTLSEKERQIKKQTA | | |
| | LVELVKHKPKATKEQLKAVMDDFAAFVEKCCKA | | |
| | DDKETCFAEEGKKLVAASQAALGLAAEPVEDNV | | |
| | INFVAMKFIDNTLYFIAEDDENDDDDKYFGKLE | | |
| | SKLSVIRNLNDQVLFIDQGNRPLFEDMTDSDVR | | |
| | DNAPRTIFIISMYKDSQPRGMAVTISVKVEKIS | | |
| | TLSVENKIISFKEMNPPDNIKDTKSDIIFFQRS | | |
| | VPGHDNKMQFESSSYEGYFLAVEKERDLFKLIL | | |
| | KKEDELGDRSIMFTVQNED (SEQ ID NO: 2) | | |
| FUSE-502 | DAHKSEVAHRFKDLGEENFKALVLIAFAQYLQQ | | |
| | CPFEDHVKLVNEVTEFAKTCVADESAENCDKSL | | |
| | HTLFGDKLCTVATLRETYGEMADCCAKQEPERN | | |
| | ECFLQHKDDNPNLPRLVRPEVDVMCTAFHDNEE | | |
| | TFLKKYLYEIARRHPYFYAPELLFFAKRYKAAF | | |
| | TECCQAADKAACLLPKLDELRDEGKASSAKQRL | | |
| | KCASLQKFGERAFKAWAVARLSQREPKAEFAEV | | |
| | SKLVTDLTKVHTECCHGDLLECADDRADLAKYI | | |
| | CENQDSISSKLKECCEKPLLEKSHCIAEVENDE | | |
| | MPADLPSLAADFVESKDVCKNYAEAKDVFLGMF | | |
| | LYEYARRHPDYSVVLLLRLAKTYETTLEKCCAA | | |
| | ADPHECYAKVEDEFKPLVEEPQNLIKQNCELFE | | |
| | QLGEYKFQNALLVRYTKKVPQVSTPTLVEVSRN | | |
| | LGKVGSKCCKHPEAKRMPCAEDYLSVVLNQLCV | | |
| | LHEKTPVSDRVTKCCTESLVNRRPCFSALEVDE | | |
| | TYVPKEFNAETFTFHADICTLSEKERQIKKQTA | | |
| | LVELVKHKPKATKEQLKAVMDDFAAFVEKCCKA | | |
| | DDKETCFAEEGKKLVAASQAALGLAAEPVEDNV | | |
| | INFVAMKFIDNTLYFIAEDDENDDDDKYFGKLE | | |
| | SKLSVIRNLNDQVLFIDQGNRPLFEDMTDSDVR | | |
| | DNAPRTIFIISKYGDSQPRGLAVTISVKVEKIS | | |
| | TLSVENKIISFKEMNPPDNIKDTKSDIIFFQRS | | |
| | VPGHDNKVQFESSSYEGYFLAVEKERDLFKLIL | | |
| | KKEDELGDRSIMFTVQNED (SEQ ID NO: 3) | | |
| FUSE-503 | DAHKSEVAHRFKDLGEENFKALVLIAFAQYLQQ | | |
| | CPFEDHVKLVNEVTEFAKTCVADESAENCDKSL | | |
| | HTLFGDKLCTVATLRETYGEMADCCAKQEPERN | | |
| | ECFLQHKDDNPNLPRLVRPEVDVMCTAFHDNEE | | |
| | TFLKKYLYEIARRHPYFYAPELLFFAKRYKAAF | | |
| | TECCQAADKAACLLPKLDELRDEGKASSAKQRL | | |
| | KCASLQKFGERAFKAWAVARLSQRFPKAEFAEV | | |
| | SKLVTDLTKVHTECCHGDLLECADDRADLAKYI | | |
| | CENQDSISSKLKECCEKPLLEKSHCIAEVENDE | | |
| | MPADLPSLAADEVESKDVCKNYAEAKDVFLGME | | |
| | LYEYARRHPDYSVVLLLRLAKTYETTLEKCCAA | | |
| | ADPHECYAKVEDEFKPLVEEPQNLIKQNCELFE | | |
| | QLGEYKFQNALLVRYTKKVPQVSTPTLVEVSRN | | |
| | LGKVGSKCCKHPEAKRMPCAEDYLSVVLNQLCV | | |
| | LHEKTPVSDRVTKCCTESLVNRRPCFSALEVDE | | |
| | TYVPKEFNAETFTFHADICTLSEKERQIKKQTA | | |
| | LVELVKHKPKATKEQLKAVMDDFAAFVEKCCKA | | |
| | DDKETCFAEEGKKLVAASQAALGLGDDDDKYFG | | |
| | KLESKLSVIRNLNDQVLFIDQGNRPLFEDMTDS | | |
| | DVRDNAPRTIFIISMYKDSQPRGMAVTISVKVE | | |
| | KISTLSVENKIISFKEMNPPDNIKDTKSDIIFF | | |
| | QRSVPGHDNKMQFESSSYEGYFLAVEKERDLFK | | |
| | LILKKEDELGDRSIMFTVQNED (SEQ ID | | |
| | NO: 4) | | |
| FUSE-504 | DAHKSEVAHRFKDLGEENFKALVLIAFAQYLQQ | | |
| | CPFEDHVKLVNEVTEFAKTCVADESAENCDKSL | | |
| | HTLFGDKLCTVATLRETYGEMADCCAKQEPERN | | |
| | ECFLQHKDDNPNLPRLVRPEVDVMCTAFHDNEE | | |
| | TFLKKYLYEIARRHPYFYAPELLFFAKRYKAAF | | |
| | TECCQAADKAACLLPKLDELRDEGKASSAKQRL | | |
| | KCASLQKFGERAFKAWAVARLSQRFPKAEFAEV | | |
| | SKLVTDLTKVHTECCHGDLLECADDRADLAKYI | | |
| | CENQDSISSKLKECCEKPLLEKSHCIAEVENDE | | |
| | MPADLPSLAADFVESKDVCKNYAEAKDVFLGMF | | |
| | LYEYARRHPDYSVVLLLRLAKTYETTLEKCCAA | | |
| | ADPHECYAKVEDEFKPLVEEPQNLIKQNCELFE | | |
| | QLGEYKFQNALLVRYTKKVPQVSTPTLVEVSRN | | |
| | LGKVGSKCCKHPEAKRMPCAEDYLSVVLNQLCV | | |
| | LHEKTPVSDRVTKCCTESLVNRRPCFSALEVDE | | |
| | TYVPKEFNAETFTFHADICTLSEKERQIKKQTA | | |
| | LVELVKHKPKATKEQLKAVMDDFAAFVEKCCKA | | |
| | DDKETCFAEEGKKLVAASQAALGLGDDDDKYFG | | |
| | KLESKLSVIRNLNDQVLFIDQGNRPLFEDMTDS | | |
| | DVRDNAPRTIFIISKYGDSQPRGLAVTISVKVE | | |
| | KISTLSVENKIISFKEMNPPDNIKDTKSDIIFF | | |
| | QRSVPGHDNKVQFESSSYEGYFLAVEKERDLFK | | |
| | LILKKEDELGDRSIMFTVQNED (SEQ ID | | |
| | NO: 5) | | |
| FUSE-505 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKDDDD | |
| | | KYFGKLESKLSVIRNLNDQVLFIDQGNRPLFEDMTDSDVRDNAPRTIFIISMYKDSQPR | |
| | | GMAVTISVKVEKISTLSVENKIISFKEMNPPDNIKDTKSDIIFFQRSVPGHDNKMQFES | |
| | | SSYEGYFLAVEKERDLFKLILKKEDELGDRSIMFTVQNED (SEQ ID NO: 34) | |
| FUSE-507 | EPKSADKTHTCPPCPAPELLGGPSVFLFPPKPK | | |
| | DTLMISRTPEVTCVVVDVSHEDPEVKFNWYVDG | | |
| | VEVHNAKTKPREEQYNSTYRVVSVLTVLHQDWL | | |
| | NGKEYKCKVSNKALPAPIEKTISKAKGQPREPQ | | |
| | VYTLPPSREEMTKNQVSLTCLVKGFYPSDIAVE | | |
| | WESNGQPENNYKTTPPVLDSDGSFFLYSKLTVD | | |
| | KSRWQQGNVFSCSVMHEALHNHYTQKSLSLSPG | | |
| | KAAEPVEDNVINFVAMKFIDNTLYFIAEDDEND | | |
| | DDDKYFGKLESKLSVIRNLNDQVLFIDQGNRPL | | |
| | FEDMTDSDVRDNAPRTIFIISMYKDSQPRGMAV | | |
| | TISVKVEKISTLSVENKIISFKEMNPPDNIKDT | | |
| | KSDIIFFQRSVPGHDNKMQFESSSYEGYFLAVE | | |
| | KERDLFKLILKKEDELGDRSIMETVQNED | | |
| | (SEQ ID NO: 6) | | |
| FUSE-508 | EPKSADKTHTCPPCPAPELLGGPSVFLFPPKPK | | |
| | DTLMISRTPEVTCVVVDVSHEDPEVKFNWYVDG | | |
| | VEVHNAKTKPREEQYNSTYRVVSVLTVLHQDWL | | |
| | NGKEYKCKVSNKALPAPIEKTISKAKGQPREPQ | | |
| | VYTLPPSREEMTKNQVSLTCLVKGFYPSDIAVE | | |
| | WESNGQPENNYKTTPPVLDSDGSFFLYSKLTVD | | |
| | KSRWQQGNVFSCSVMHEALHNHYTQKSLSLSPG | | |
| | KDDDDKYFGKLESKLSVIRNLNDQVLFIDQGNR | | |
| | PLFEDMTDSDVRDNAPRTIFIISMYKDSQPRGM | | |
| | AVTISVKVEKISTLSVENKIISFKEMNPPDNIK | | |
| | DTKSDIIFFQRSVPGHDNKMQFESSSYEGYFLA | | |
| | VEKERDLFKLILKKEDELGDRSIMETVQNED | | |
| | (SEQ ID NO: 7) | | |
| FUSE-509 | ESKYGPPCPSCPAPEFLGGPSVFLFPPKPKDTL | | |
| | MISRTPEVTCVVVDVSQEDPEVQFNWYVDGVEV | | |
| | HNAKTKPREEQFNSTYRVVSVLTVLHQDWLNGK | | |
| | EYKCKVSNKGLPSSIEKTISKAKGQPREPQVYT | | |
| | LPPSQEEMTKNQVSLTCLVKGFYPSDIAVEWES | | |
| | NGQPENNYKTTPPVLDSDGSFFLYSRLTVDKSR | | |
| | WQEGNVFSCSVMHEALHNHYTQKSLSLSLGKAA | | |
| | EPVEDNVINFVAMKFIDNTLYFIAEDDENDDDD | | |
| | KYFGKLESKLSVIRNLNDQVLFIDQGNRPLFED | | |
| | MTDSDVRDNAPRTIFIISMYKDSQPRGMAVTIS | | |
| | VKVEKISTLSVENKIISFKEMNPPDNIKDTKSD | | |
| | IIFFQRSVPGHDNKMQFESSSYEGYFLAVEKER | | |
| | DLFKLILKKEDELGDRSIMFTVQNED (SEQ | | |
| | ID NO: 8) | | |
| FUSE-510 | ESKYGPPCPSCPAPEFLGGPSVFLFPPKPKDTL | | |
| | MISRTPEVTCVVVDVSQEDPEVQFNWYVDGVEV | | |
| | HNAKTKPREEQFNSTYRVVSVLTVLHQDWLNGK | | |
| | EYKCKVSNKGLPSSIEKTISKAKGQPREPQVYT | | |
| | LPPSQEEMTKNQVSLTCLVKGFYPSDIAVEWES | | |
| | NGQPENNYKTTPPVLDSDGSFFLYSRLTVDKSR | | |
| | WQEGNVFSCSVMHEALHNHYTQKSLSLSLGKDD | | |
| | DDKYFGKLESKLSVIRNLNDQVLFIDQGNRPLF | | |
| | EDMTDSDVRDNAPRTIFIISMYKDSQPRGMAVT | | |
| | ISVKVEKISTLSVENKIISFKEMNPPDNIKDTK | | |
| | SDIIFFQRSVPGHDNKMQFESSSYEGYFLAVEK | | |
| | ERDLFKLILKKEDELGDRSIMETVQNED (SEQ | | |
| | ID NO: 9) | | |
| FUSE-516 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMNSLKPEDTAVYYCAAGYQINSGNYNEKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNVINEVAMKFIDNTLYFIAEDDENIEQDYFGKLESKLSVIRNLNDQV | |
| | | LFIDQGNRPLFEDMTDSDVRDNAPRTIFIISKYGDSQPRGLAVTISVKVEKISTLSVEN | |
| | | KIISFKEMNPPDNIKDTKSDIIFFQRSVPGHDNKVQFESSSYEGYFLAVEKERDLFKLI | |
| | | LKKEDELGDRSIMFTVQNED (SEQ ID NO: 35) | |
| FUSE-517 | (SEQ ID NO: 1) | QVQLKQSGPGLVQPSQSLSITCTVSGFSLTNYGVHWVRQSPGKGLEWLGVIWSGGNTDY | SEQ |
| | | NTPFTSRLSINKDNSKSQVFFKMNSLQSNDTAIYYCARALTYYDYEFAYWGQGTLVTVS | ID |
| | | AASTKGPSVFPLAPSSKSTSGGTAALGCLVKDYFPEPVTVSWNSGALTSGVHTFPAVLQ | NO: 73 |
| | | SSGLYSLSSVVTVPSSSLGTQTYICNVNHKPSNTKVDKKVEPKSCDKTHTCPPCPAPEL | |
| | | LGGPSVFLFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPREPQVYTLP | |
| | | PSREEMTKNQVSLWCLVKGFYPSDIAVEWESNGQPENNYKTTPPVLDSDGSFFLYSKLT | |
| | | VDKSRWQQGNVFSCSVMHEALHNHYTQKSLSLSPGKAAEPVEDNVINFVAMKFIDNTLY | |
| | | FIAEDDENIEQDYFGKLESKLSVIRNLNDQVLFIDQGNRPLFEDMTDSDVRDNAPRTIF | |
| | | IISKYGDSQPRGLAVTISVKVEKISTLSVENKIISFKEMNPPDNIKDTKSDIIFFQRSV | |
| | | PGHDNKVQFESSSYEGYFLAVEKERDLFKLILKKEDELGDRSIMETVQNED (SEQ ID | |
| | | NO: 36) | |
| FUSE-545 | (SEQ ID NO: 1) | YFGKLESKLSVIRNLNDQVLFIDQGNRPLFEDMTDSDVRDNAPRTIFIISKYGDSQPRG | |
| | | LAVTISVKVEKISTLSVENKIISFKEMNPPDNIKDTKSDIIFFQRSVPGHDNKVQFESS | |
| | | SYEGYFLAVEKERDLFKLILKKEDELGDRSIMETVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 37) | |
| FUSE-546 | (SEQ ID NO: 1) | YFGKLKSKLSVIRNLNDQVLFIDQGNRPLFEDMTDSDVRDNAPRTIFIISMYgDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 38) | |
| FUSE-547 | (SEQ ID NO: 1) | YFGKLrSKLSVIRNLNDQVLFIDQGNRPLFEDMTDSDVRDNAPRTIFIISMYgDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 39) | |
| FUSE-555 | QVQLKQSGPGLVQPSQSLSITCTVSGFSLTNYG | QVQLKQSGPGLVQPSQSLSITCTVSGFSLTNYGVHWVRQSPGKGLEWLGVIWSGGNTDY | SEQ |
| | VHWVRQSPGKGLEWLGVIWSGGNTDYNTPFTSR | NTPFTSRLSINKDNSKSQVFFKMNSLQSNDTAIYYCARALTYYDYEFAYWGQGTLVTVS | ID |
| | LSINKDNSKSQVFFKMNSLQSNDTAIYYCARAL | AASTKGPSVFPLAPSSKSTSGGTAALGCLVKDYFPEPVTVSWNSGALTSGVHTFPAVLQ | NO: 73 |
| | TYYDYEFAYWGQGTLVTVSAASTKGPSVEPLAP | SSGLYSLSSVVTVPSSSLGTQTYICNVNHKPSNTKVDKKVEPKSCDKTHTCPPCPAPEL | |
| | SSKSTSGGTAALGCLVKDYFPEPVTVSWNSGAL | LGGPSVFLFPPKPKDTLMISRTPEVTCVVVDVSHEDPEVKFNWYVDGVEVHNAKTKPRE | |
| | TSGVHTFPAVLQSSGLYSLSSVVTVPSSSLGTQ | EQYNSTYRVVSVLTVLHQDWLNGKEYKCKVSNKALPAPIEKTISKAKGQPREPQVYTLP | |
| | TYICNVNHKPSNTKVDKKVEPKSCDKTHTCPPC | PSREEMTKNQVSLWCLVKGFYPSDIAVEWESNGQPENNYKTTPPVLDSDGSFFLYSKLT | |
| | PAPELLGGPSVFLFPPKPKDTLMISRTPEVTCV | VDKSRWQQGNVFSCSVMHEALHNHYTQKSLSLSPGKGGGGSGGGGSGGGGSGGGGSAAE | |
| | VVDVSHEDPEVKFNWYVDGVEVHNAKTKPREEQ | PVEDNVINFVAMKFIDNTLYFIAEDDENIEQDYFGKLESKLSVIRNLNDQVLFIDQGNR | |
| | YNSTYRVVSVLTVLHQDWLNGKEYKCKVSNKAL | PLFEDMTDSDVRDNAPRTIFIISKYGDSQPRGLAVTISVKVEKISTLSVENKIISFKEM | |
| | PAPIEKTISKAKGQPREPQVYTLPPSREEMTKN | NPPDNIKDTKSDIIFFQRSVPGHDNKVQFESSSYEGYFLAVEKERDLFKLILKKEDELG | |
| | QVSLSCAVKGFYPSDIAVEWESNGQPENNYKTT | DRSIMETVQNED (SEQ ID NO: 40) | |
| | PPVLDSDGSFFLVSKLTVDKSRWQQGNVFSCSV | | |
| | MHEALHNRFTQKSLSLSPGK (SEQ ID | | |
| | NO: 10) | | |
| FUSE-556 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMNSLKPEDTAVYYCAAGYQINSGNYNFKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGK (SEQ ID NO: 41) | |
| FUSE-557 | (SEQ ID NO: 1 | QVQLKQSGPGLVQPSQSLSITCTVSGFSLTNYGVHWVRQSPGKGLEWLGVIWSGGNTDY | SEQ |
| | | NTPFTSRLSINKDNSKSQVFFKMNSLQSNDTAIYYCARALTYYDYEFAYWGQGTLVTVS | ID |
| | | AASTKGPSVFPLAPSSKSTSGGTAALGCLVKDYFPEPVTVSWNSGALTSGVHTFPAVLQ | NO: 73 |
| | | SSGLYSLSSVVTVPSSSLGTQTYICNVNHKPSNTKVDKKVEPKSCDKTHTCPPCPAPEL | |
| | | LGGPSVFLFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPREPQVYTLP | |
| | | PSREEMTKNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK (SEQ ID NO: 254) | |
| FUSE-583 | (SEQ ID NO: 1) | YFGKLaSKLSVIRNLNDQVLFIDQGNRPLFEDMTDSDVRDNAPRTIFIISMYaDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 42) | |
| FUSE-584 | (SEQ ID NO: 1) | YFGKLhSKLSVIRNLNDQVLFIDQGNRPLFEDMTDSDVRDNAPRTIFIISMYgDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 43) | |
| FUSE-585 | (SEQ ID NO: 1) | YFGKLrSKLSVIRNLNDQVLFIDQGNRPLFEDMTDSDVRDNAPRTIFIISMYeDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 44) | |
| FUSE-586 | (SEQ ID NO: 1) | YFGKLqSKLSVIRNLNDQVLFIDQGNRPLFEDMTDSDVRDNAPRTIFIISMYaDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 45) | |
| FUSE-587 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNVINFVAMKFIDNTLYFIAEDDENGGGGKPLGLQARVVGGGGTIEQDYFGKLESKL | |
| | | SVIRNLNDQVLFIDQGNRPLFEDMTDSDVRDNAPRTIFIISMYKDSQPRGMAVTISVKV | |
| | | EKISTLSVENKIISFKEMNPPDNIKDTKSDIIFFQRSVPGHDNKMQFESSSYEGYFLAV | |
| | | EKERDLFKLILKKEDELGDRSIMETVQNED (SEQ ID NO: 46) | |
| FUSE-599 | (SEQ ID NO: 1) | YFGKLKSKLSVIRNLNDQVLFIDQGNRPLFEDMTDSDVRDNAPRTIFIIShYgDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 47) | |
| FUSE-600 | (SEQ ID NO: 1) | YFGKLKSKLSVIRNLNDQVLFIDQGNRPLFEDMTDSDVRDNAPRTIFIISKYgDSQPRG | |
| | | MAVTISVKVEKISTLSVENKIISFKEMNPPDNIKDTKSDIIFFQRSVPGHDNKMQFESS | |
| | | SYEGYFLAVEKERDLFKLILKKEDELGDRSIMETVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 48) | |
| FUSE-601 | (SEQ ID NO: 1) | YFGKLESKLSVIRNLNDQVLFIDQGNRPLFEDMTDSDVRDNAPRTIFIIShYaDSQPRG | |
| | | iAVTISVKVEKISTLSVENKIISFKEMNPPDNIKDTKSDIIFFQRSVPGHDNKIQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 49) | |
| FUSE-602 | (SEQ ID NO: 1) | YFGKLESKLSVIRNLNDQVLFIDQGNRPLFEDMTDSDVRDNAPRTIFIISKYaDSQPRG | |
| | | iAVTISVKVEKISTLSVENKIISFKEMNPPDNIKDTKSDIIFFQRSVPGHDNKIQFESS | |
| | | SYEGYFLAVEKERDLFKLILKKEDELGDRSIMFTVQNEDEPKSSDKTHTCPPCPAPELL | |
| | | GGPSVFLFPPKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPREPQVYTLPP | |
| | | SREEMTKNQVSLWCLVKGFYPSDIAVEWESNGQPENNYKTTPPVLDSDGSFFLYSKLTV | |
| | | DKSRWQQGNVFSCSVMHEALHNHYTQKSLSLSPGK (SEQ ID NO: 50) | |
| FUSE-624 | (SEQ ID NO: 10) | QVQLKQSGPGLVQPSQSLSITCTVSGFSLTNYGVHWVRQSPGKGLEWLGVIWSGGNTDY | SEQ |
| | | NTPFTSRLSINKDNSKSQVFFKMNSLQSNDTAIYYCARALTYYDYEFAYWGQGTLVTVS | ID |
| | | AASTKGPSVFPLAPSSKSTSGGTAALGCLVKDYFPEPVTVSWNSGALTSGVHTEPAVLQ | NO: 73 |
| | | SSGLYSLSSVVTVPSSSLGTQTYICNVNHKPSNTKVDKKVEPKSCDKTHTCPPCPAPEL | |
| | | LGGPSVFLFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPREPQVYTLP | |
| | | PSREEMTKNQVSLWCLVKGFYPSDIAVEWESNGQPENNYKTTPPVLDSDGSFFLYSKLT | |
| | | VDKSRWQQGNVFSCSVMHEALHNHYTQKSLSLSPGKGGGGSGGGGSGGGGSGGGGSAAE | |
| | | PVEDNVINFVAMKFIDNTLYFIAEDDENGGGGKPLGLQARVVGGGGTIEQDYFGKLESK | |
| | | LSVIRNLNDQVLFIDQGNRPLFEDMTDSDVRDNAPRTIFIISKYGDSQPRGLAVTISVK | |
| | | VEKISTLSVENKIISFKEMNPPDNIKDTKSDIIFFQRSVPGHDNKVQFESSSYEGYFLA | |
| | | VEKERDLFKLILKKEDELGDRSIMETVQNED (SEQ ID NO: 51) | |
| FUSE-625 | EVQLVESGGGLVQPGGSLRLSCAASGFNIKDTY | EVQLVESGGGLVQPGGSLRLSCAASGENIKDTYIHWVRQAPGKGLEWVARIYPTNGYTR | SEQ |
| | IHWVRQAPGKGLEWVARIYPTNGYTRYADSVKG | YADSVKGRFTISADTSKNTAYLQMNSLRAEDTAVYYCSRWGGDGFYAMDYWGQGTLVTV | ID |
| | RFTISADTSKNTAYLQMNSLRAEDTAVYYCSRW | SSASTKGPSVFPLAPSSKSTSGGTAALGCLVKDYFPEPVTVSWNSGALTSGVHTFPAVL | NO: 74 |
| | GGDGFYAMDYWGQGTLVTVSSASTKGPSVEPLA | QSSGLYSLSSVVTVPSSSLGTQTYICNVNHKPSNTKVDKKVEPKSCDKTHTCPPCPAPE | |
| | PSSKSTSGGTAALGCLVKDYFPEPVTVSWNSGA | LLGGPSVFLFPPKPKDTLMISRTPEVTCVVVDVSHEDPEVKFNWYVDGVEVHNAKTKPR | |
| | LTSGVHTFPAVLQSSGLYSLSSVVTVPSSSLGT | EEQYNSTYRVVSVLTVLHQDWLNGKEYKCKVSNKALPAPIEKTISKAKGQPREPQVYTL | |
| | QTYICNVNHKPSNTKVDKKVEPKSCDKTHTCPP | PPSREEMTKNQVSLWCLVKGFYPSDIAVEWESNGQPENNYKTTPPVLDSDGSFFLYSKL | |
| | CPAPELLGGPSVFLFPPKPKDTLMISRTPEVTC | TVDKSRWQQGNVFSCSVMHEALHNHYTQKSLSLSPGKGGGGSGGGGSGGGGSGGGGSAA | |
| | VVVDVSHEDPEVKFNWYVDGVEVHNAKTKPREE | EPVEDNVINFVAMKFIDNTLYFIAEDDENGGGGKPLGLQARVVGGGGTIEQDYFGKLES | |
| | QYNSTYRVVSVLTVLHQDWLNGKEYKCKVSNKA | KLSVIRNLNDQVLFIDQGNRPLFEDMTDSDVRDNAPRTIFIISKYGDSQPRGLAVTISV | |
| | LPAPIEKTISKAKGQPREPQVYTLPPSREEMTK | KVEKISTLSVENKIISFKEMNPPDNIKDTKSDIIFFQRSVPGHDNKVQFESSSYEGYEL | |
| | NQVSLSCAVKGFYPSDIAVEWESNGQPENNYKT | AVEKERDLFKLILKKEDELGDRSIMFTVQNED (SEQ ID NO: 52) | |
| | TPPVLDSDGSFFLVSKLTVDKSRWQQGNVFSCS | | |
| | VMHEALHNRFTQKSLSLSPGK (SEQ ID | | |
| | NO: 11) | | |
| FUSE-626 | (SEQ ID NO: 1) | EVQLVESGGGLVQAGGSLRLSCAASGRTFSSYAMGWFRQAPGKEREFVVAINWSSGSTY | |
| | | YADSVKGRFTISRDNAKNTMYLQMNSLKPEDTAVYYCAAGYQINSGNYNEKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNVINFVAMKFIDNTLYFIAEDDENGGGSYFGKLESKLSVIRNLNDQV | |
| | | LFIDQGNRPLFEDMTDSDVRDNAPRTIFIISKYGDSQPRGLAVTISVKVEKISTLSVEN | |
| | | KIISFKEMNPPDNIKDTKSDIIFFQRSVPGHDNKVQFESSSYEGYFLAVEKERDLFKLI | |
| | | LKKEDELGDRSIMFTVQNED (SEQ ID NO: 257) | |
| FUSE-627 | EPKSSDKTHTCPPCPAPEaaGGPSVFLFPPKPK | EVQLVESGGGLVQAGGSLRLSCAASGRTESSYAMGWFRQAPGKEREFVVAINWSSGSTY | |
| | DTLMISRTPEVTCVVVDVSHEDPEVKFNWYVDG | YADSVKGRFTISRDNAKNTMYLQMNSLKPEDTAVYYCAAGYQINSGNYNEKDYEYDYWG | |
| | VEVHNAKTKPREEQYNSTYRVVSVLTVLHQDWL | QGTQVTVSSEPKSSDKTHTCPPCPAPEaaGGPSVFLFPPKPKDTLMISRTPEVTCVVVD | |
| | NGKEYKCKVSNKALgAPIEKTISKAKGQPREPQ | VSHEDPEVKFNWYVDGVEVHNAKTKPREEQYNSTYRVVSVLTVLHQDWLNGKEYKCKVS | |
| | VYTLPPSREEMTKNQVSLSCAVKGFYPSDIAVE | NKALgAPIEKTISKAKGQPREPQVYTLPPSREEMTKNQVSLWCLVKGFYPSDIAVEWES | |
| | WESNGQPENNYKTTPPVLDSDGSFFLVSKLTVD | NGQPENNYKTTPPVLDSDGSFFLYSKLTVDKSRWQQGNVFSCSVMHEALHNHYTQKSLS | |
| | KSRWQQGNVFSCSVMHEALHNRFTQKSLSLSPG | LSPGKAAEPVEDNVINFVAMKFIDNTLYFIAEDDENIEQDYFGKLESKLSVIRNLNDQV | |
| | K (SEQ ID NO: 12) | LFIDQGNRPLFEDMTDSDVRDNAPRTIFIISKYGDSQPRGLAVTISVKVEKISTLSVEN | |
| | | KIISFKEMNPPDNIKDTKSDIIFFQRSVPGHDNKVQFESSSYEGYFLAVEKERDLEKLI | |
| | | LKKEDELGDRSIMETVQNED (SEQ ID NO: 53) | |
| FUSE-645 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMNSLKPEDTAVYYCAAGYQINSGNYNEKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNSINFVAMKFIDNTLYFIAEDDENIEQDYFGKLESKLSVIRNLNDQV | |
| | | LFIDQGNRPLFEDMTDSDSRDNAPRTIFIISKYGDSQPRGLAVTISVKSEKISTLSSEN | |
| | | KIISFKEMNPPDNIKDTKSDIIFFQRSVPGHDNKVQFESSSYEGYFLASEKERDLEKLI | |
| | | LKKEDELGDRSIMETVQNED (SEQ ID NO: 54) | |
| FUSE-658 | EPKSSDKTHTCPPCPAPELLGGPSVFLFPPKPK | SEQ ID NO: 41 | |
| | DTLMISRTPEVTCVVVDVSHEDPEVKFNWYVDG | | |
| | VEVHNAKTKPREEQYNSTYRVVSVLTVLHQDWL | | |
| | NGKEYKCKVSNKALPAPIEKTISKAKGQPREPQ | | |
| | VYTLPPSREEMTKNQVSLSCAVKGFYPSDIAVE | | |
| | WESNGQPENNYKTTPPVLDSDGSFFLVSKLTVD | | |
| | KSRWQQGNVFSCSVMHEALHNRFTQKSLSLSPG | | |
| | KAAEPVEDNVINFVAMKFIDNTLYFIAEDDENI | | |
| | GNRSYFGKLESKLSVIRNLNDQVLFIDQGNRPL | | |
| | FEDMTDSDVRDNAPRTIFIISKYGDSQPRGLAV | | |
| | TISVKVEKISTLSVENKIISFKEMNPPDNIKDT | | |
| | KSDIIFFQRSVPGHDNKVQFESSSYEGYFLAVE | | |
| | KERDLFKLILKKEDELGDRSIMETVQNED | | |
| | (SEQ ID NO: 13) | | |
| FUSE-659 | EPKSSDKTHTCPPCPAPELLGGPSVFLFPPKPK | SEQ ID NO: 41 | |
| | DTLMISRTPEVTCVVVDVSHEDPEVKFNWYVDG | | |
| | VEVHNAKTKPREEQYNSTYRVVSVLTVLHQDWL | | |
| | NGKEYKCKVSNKALPAPIEKTISKAKGQPREPQ | | |
| | VYTLPPSREEMTKNQVSLSCAVKGFYPSDIAVE | | |
| | WESNGQPENNYKTTPPVLDSDGSFFLVSKLTVD | | |
| | KSRWQQGNVFSCSVMHEALHNRFTQKSLSLSPG | | |
| | KAAEPVEDNVINFVAMKFIDNTLYFIAEDDENG | | |
| | GGGKPLGLQARVVGGGGTYFGKLESKLSVIRNL | | |
| | NDQVLFIDQGNRPLFEDMTDSDVRDNAPRTIFI | | |
| | ISKYGDSQPRGLAVTISVKVEKISTLSVENKII | | |
| | SFKEMNPPDNIKDTKSDIIFFQRSVPGHDNKVQ | | |
| | FESSSYEGYFLAVEKERDLFKLILKKEDELGDR | | |
| | SIMETVQNED (SEQ ID NO: 14) | | |
| FUSE-660 | DTLMISRTPEVTCVVVDVSHEDPEVKFNWYVDG | SEQ ID NO: 41 | |
| | EPKSSDKTHTCPPCPAPELLGGPSVFLFPPKPK | | |
| | VEVHNAKTKPREEQYNSTYRVVSVLTVLHQDWL | | |
| | NGKEYKCKVSNKALPAPIEKTISKAKGQPREPQ | | |
| | VYTLPPSREEMTKNQVSLSCAVKGFYPSDIAVE | | |
| | WESNGQPENNYKTTPPVLDSDGSFFLVSKLTVD | | |
| | KSRWQQGNVFSCSVMHEALHNRFTQKSLSLSPG | | |
| | KAAEPVEDNVINFVAMKFIDNTLYFIAEDDENI | | |
| | EQDGGSIGNRSGGGGKPLGLQARVVGGGGTIEQ | | |
| | DYFGKLESKLSVIRNLNDQVLFIDQGNRPLFED | | |
| | MTDSDVRDNAPRTIFIISKYGDSQPRGLAVTIS | | |
| | VKVEKISTLSVENKIISFKEMNPPDNIKDTKSD | | |
| | IIFFQRSVPGHDNKVQFESSSYEGYFLAVEKER | | |
| | DLFKLILKKEDELGDRSIMFTVQNED (SEQ | | |
| | ID NO: 15) | | |
| FUSE-669 | (SEQ ID NO: 10) | SEQ ID NO: 254 | SEQ |
| | | | ID |
| | | | NO: 73 |
| FUSE-670 | QVQLKQSGPGLVQPSQSLSITCTVSGESLTNYG | | SEQ |
| | VHWVRQSPGKGLEWLGVIWSGGNTDYNTPFTSR | | ID |
| | LSINKDNSKSQVFFKMNSLQSNDTAIYYCARAL | | NO: 73 |
| | TYYDYEFAYWGQGTLVTVSAASTKGPSVEPLAP | | |
| | SSKSTSGGTAALGCLVKDYFPEPVTVSWNSGAL | | |
| | TSGVHTFPAVLQSSGLYSLSSVVTVPSSSLGTQ | | |
| | TYICNVNHKPSNTKVDKKVEPKSCDKTHTCPPC | | |
| | PAPELLGGPSVFLFPPKPKDTLMISRTPEVTCV | | |
| | VVDVSHEDPEVKFNWYVDGVEVHNAKTKPREEQ | | |
| | YNSTYRVVSVLTVLHQDWLNGKEYKCKVSNKAL | | |
| | PAPIEKTISKAKGQPREPQVYTLPPSRDELTKN | | |
| | QVSLTCLVKGFYPSDIAVEWESNGQPENNYKTT | | |
| | PPVLDSDGSFFLYSKLTVDKSRWQQGNVFSCSV | | |
| | MHEALHNHYTQKSLSLSPGKGGGGSGGGGSGGG | | |
| | GSGGGGSGGGGSGGGGSAAEPVEDNVINEVAMK | | |
| | FIDNTLYFIAEDDENIEQDYFGKLESKLSVIRN | | |
| | LNDQVLFIDQGNRPLFEDMTDSDVRDNAPRTIF | | |
| | IISKYGDSQPRGLAVTISVKVEKISTLSVENKI | | |
| | ISFKEMNPPDNIKDTKSDIIFFQRSVPGHDNKV | | |
| | QFESSSYEGYFLAVEKERDLFKLILKKEDELGD | | |
| | RSIMFTVQNED (SEQ ID NO: 255) | | |
| FUSE-675 | EVQLVESGGGLVQAGGSLRLSCAASGRTFSSYA | | |
| | MGWFRQAPGKEREFVVAINWSSGSTYYADSVKG | | |
| | RFTISRDNAKNTMYLQMSSLKPEDTAVYYCAAG | | |
| | YQINSGNYNFKDYEYDYWGQGTQVTVSSEPKSA | | |
| | DKTHTCPPCPAPELLGGPSVFLFPPKPKDTLMI | | |
| | SRTPEVTCVVVDVSHEDPEVKFNWYVDGVEVHN | | |
| | AKTKPREEQYNSTYRVVSVLTVLHQDWLNGKEY | | |
| | KCKVSNKALPAPIEKTISKAKGQPREPQVYTLP | | |
| | PSREEMTKNQVSLTCLVKGFYPSDIAVEWESNG | | |
| | QPENNYKTTPPVLDSDGSFFLYSKLTVDKSRWQ | | |
| | QGNVFSCSVMHEALHNHYTQKSLSLSPGK | | |
| | (SEQ ID NO: 256) | | |
| FUSE-676 | EVQLVESGGGLVQAGGSLRLSCAASGRTFSSYA | | |
| | MGWFRQAPGKEREFVVAINWSSGSTYYADSVKG | | |
| | RFTISRDNAKNTMYLQMSSLKPEDTAVYYCAAG | | |
| | YQINSGNYNFKDYEYDYWGQGTQVTVSSEPKSA | | |
| | DKTHTCPPCPAPELLGGPSVFLFPPKPKDTLMI | | |
| | SRTPEVTCVVVDVSHEDPEVKFNWYVDGVEVHN | | |
| | AKTKPREEQYNSTYRVVSVLTVLHQDWLNGKEY | | |
| | KCKVSNKALPAPIEKTISKAKGQPREPQVYTLP | | |
| | PSREEMTKNQVSLTCLVKGFYPSDIAVEWESNG | | |
| | QPENNYKTTPPVLDSDGSFFLYSKLTVDKSRWQ | | |
| | QGNVFSCSVMHEALHNHYTQKSLSLSPGKGGGG | | |
| | SGGGGSGGGGSGGGGSGGGGSGGGGSAAEPVED | | |
| | NVINFVAMKFIDNTLYFIAEDDENIEQDYFGKL | | |
| | ESKLSVIRNLNDQVLFIDQGNRPLFEDMTDSDV | | |
| | RDNAPRTIFIISKYGDSQPRGLAVTISVKVEKI | | |
| | STLSVENKIISFKEMNPPDNIKDTKSDIIFFQR | | |
| | SVPGHDNKVQFESSSYEGYFLAVEKERDLEKLI | | |
| | LKKEDELGDRSIMFTVQNED (SEQ ID | | |
| | NO: 267) | | |
| FUSE-682 | EVQLVESGGGLVQAGGSLRLSCAASGRTFSSYA | SEQ ID NO: 35 | |
| | MGWFRQAPGKEREFVVAINWSSGSTYYADSVKG | | |
| | RFTISRDNAKNTMYLQMSSLKPEDTAVYYCAAG | | |
| | YQINSGNYNFKDYEYDYWGQGTQVTVSSEPKSA | | |
| | DKTHTCPPCPAPELLGGPSVFLFPPKPKDTLMI | | |
| | SRTPEVTCVVVDVSHEDPEVKFNWYVDGVEVHN | | |
| | AKTKPREEQYNSTYRVVSVLTVLHQDWLNGKEY | | |
| | KCKVSNKALPAPIEKTISKAKGQPREPQVYTLP | | |
| | PSREEMTKNQVSLSCAVKGFYPSDIAVEWESNG | | |
| | QPENNYKTTPPVLDSDGSFFLVSKLTVDKSRWQ | | |
| | QGNVFSCSVMHEALHNRFTQKSLSLSPGK | | |
| | (SEQ ID NO: 258) | | |
| FUSE-683 | EPKSADKTHTCPPCPAPELLGGPSVFLFPPKPK | EVQLVESGGGLVQAGGSLRLSCAASGRTFSSYAMGWFRQAPGKEREFVVAINWSSGSTY | |
| | DTLMISRTPEVTCVVVDVSHEDPEVKFNWYVDG | YADSVKGRFTISRDNAKNTMYLQMSSLKPEDTAVYYCAAGYQINSGNYNEKDYEYDYWG | |
| | VEVHNAKTKPREEQYNSTYRVVSVLTVLHQDWL | QGTQVTVSSEPKSSDKTHTCPPCPAPELLGGPSVFLFPPKPKDTLMISRTPEVTCVVVD | |
| | NGKEYKCKVSNKALPAPIEKTISKAKGQPREPQ | VSHEDPEVKFNWYVDGVEVHNAKTKPREEQYNSTYRVVSVLTVLHQDWLNGKEYKCKVS | |
| | VYTLPPSREEMTKNQVSLSCAVKGFYPSDIAVE | NKALPAPIEKTISKAKGQPREPQVYTLPPSREEMTKNQVSLWCLVKGFYPSDIAVEWES | |
| | WESNGQPENNYKTTPPVLDSDGSFFLVSKLTVD | NGQPENNYKTTPPVLDSDGSFFLYSKLTVDKSRWQQGNVFSCSVMHEALHNHYTQKSLS | |
| | KSRWQQGNVFSCSVMHEALHNRFTQKSLSLSPG | LSPGKGGGGSGGGGSGGGGSGGGGSGGGGSGGGGSAAEPVEDNVINFVAMKFIDNTLYF | |
| | KGGGGSGGGGSGGGGSGGGGSGGGGSGGGGSAA | IAEDDENIEQDYFGKLESKLSVIRNLNDQVLFIDQGNRPLFEDMTDSDVRDNAPRTIFI | |
| | EPVEDNVINFVAMKFIDNTLYFIAEDDENIEQD | ISKYGDSQPRGLAVTISVKVEKISTLSVENKIISFKEMNPPDNIKDTKSDIIFFQRSVP | |
| | YFGKLESKLSVIRNLNDQVLFIDQGNRPLFEDM | GHDNKVQFESSSYEGYFLAVEKERDLFKLILKKEDELGDRSIMFTVQNED (SEQ ID | |
| | TDSDVRDNAPRTIFIISKYGDSQPRGLAVTISV | NO: 272) | |
| | KVEKISTLSVENKIISFKEMNPPDNIKDTKSDI | | |
| | IFFQRSVPGHDNKVQFESSSYEGYFLAVEKERD | | |
| | LFKLILKKEDELGDRSIMFTVQNED (SEQ ID | | |
| | NO: 259) | | |
| FUSE-684 | SEQ ID NO: 259 | QVQLKQSGPGLVQPSQSLSITCTVSGESLTNYGVHWVRQSPGKGLEWLGVIWSGGNTDY | |
| | | NTPFTSRLSINKDNSKSQVFFKMNSLQSNDTAIYYCARALTYYDYEFAYWGQGTLVTVS | |
| | | AASTKGPSVFPLAPSSKSTSGGTAALGCLVKDYFPEPVTVSWNSGALTSGVHTEPAVLQ | |
| | | SSGLYSLSSVVTVPSSSLGTQTYICNVNHKPSNTKVDKKVEPKSCDKTHTCPPCPAPEL | |
| | | LGGPSVFLFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPREPQVYTLP | |
| | | PSREEMTKNQVSLWCLVKGFYPSDIAVEWESNGQPENNYKTTPPVLDSDGSFFLYSKLT | |
| | | VDKSRWQQGNVFSCSVMHEALHNHYTQKSLSLSPGKGGGGSGGGGSGGGGSGGGGSGGG | |
| | | GSGGGGSAAEPVEDNVINFVAMKFIDNTLYFIAEDDENIEQDYFGKLESKLSVIRNLND | |
| | | QVLFIDQGNRPLFEDMTDSDVRDNAPRTIFIISKYGDSQPRGLAVTISVKVEKISTLSV | |
| | | ENKIISFKEMNPPDNIKDTKSDIIFFQRSVPGHDNKVQFESSSYEGYFLAVEKERDLEK | |
| | | LILKKEDELGDRSIMFTVQNED (SEQ ID NO: 273) | |
| FUSE-685 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMNSLKPEDTAVYYCAAGYQINSGNYNEKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNSINFVAMKFIDNTLYFIAEDDENQDIEYFGKLESKLSVIRNLNDQV | |
| | | LFIDQGNRPLFEDMTDSDSRDNAPRTIFIISKYGDSQPRGLAVTISVKSEKISTLSSEN | |
| | | KIISFKEMNPPDNIKDTKSDIIFFQRSVPGHDNKVQFESSSYEGYFLASEKERDLFKLI | |
| | | LKKEDELGDRSIMFTVQNED (SEQ ID NO: 275) | |
| FUSE-686 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMNSLKPEDTAVYYCAAGYQINSGNYNFKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNSINFVAMKFIDNTLYFIAEnDENQDIEYFGKLESKLSVIRNLNDQV | |
| | | LFIDQGNRPLFEDMTDSDSRDNAPRTIFIISKYGDSQPRGLAVTISVKSEKISTLSSEN | |
| | | KIISFKEMNPPDNIKDTKSDIIFFQRSVPGHDNKVQFESSSYEGYFLASEKERDLEKLI | |
| | | LKKEDELGDRSIMETVQNED (SEQ ID NO: 55) | |
| FUSE-691 | QVQLVESGGGVVQPGRSLRLDCKASGITFSNSG | | SEQ |
| | MHWVRQAPGKGLEWVAVIWYDGSKRYYADSVKG | | ID |
| | RFTISRDNSKNTLFLQMNSLRAEDTAVYYCATN | | NO: 75 |
| | DDYWGQGTLVTVSSASTKGPSVFPLAPSSKSTS | | |
| | GGTAALGCLVKDYFPEPVTVSWNSGALTSGVHT | | |
| | FPAVLQSSGLYSLSSVVTVPSSSLGTQTYICNV | | |
| | NHKPSNTKVDKKVEPKSCDKTHTCPPCPAPEaa | | |
| | GGPSVFLFPPKPKDTLMISRTPEVTCVVVDVSH | | |
| | EDPEVKFNWYVDGVEVHNAKTKPREEQYNSTYR | | |
| | VVSVLTVLHQDWLNGKEYKCKVSNKALPAPIEK | | |
| | TISKAKGQPREPQVYTLPPSREEMTKNQVSLTC | | |
| | LVKGFYPSDIAVEWESNGQPENNYKTTPPVLDS | | |
| | DGSFFLYSKLTVDKSRWQQGNVFSCSVMHEALH | | |
| | NHYTQKSLSLSPGK (SEQ ID NO: 16) | | |
| FUSE-694 | QVQLVESGGGVVQPGRSLRLDCKASGITFSNSG | QVQLVESGGGVVQPGRSLRLDCKASGITFSNSGMHWVRQAPGKGLEWVAVIWYDGSKRY | SEQ |
| | MHWVRQAPGKGLEWVAVIWYDGSKRYYADSVKG | YADSVKGRFTISRDNSKNTLFLQMNSLRAEDTAVYYCATNDDYWGQGTLVTVSSASTKG | ID |
| | RFTISRDNSKNTLFLQMNSLRAEDTAVYYCATN | PSVFPLAPSSKSTSGGTAALGCLVKDYFPEPVTVSWNSGALTSGVHTFPAVLQSSGLYS | NO: 75 |
| | DDYWGQGTLVTVSSASTKGPSVFPLAPSSKSTS | LSSVVTVPSSSLGTQTYICNVNHKPSNTKVDKKVEPKSCDKTHTCPPCPAPEaaGGPSV | |
| | GGTAALGCLVKDYFPEPVTVSWNSGALTSGVHT | FLFPPKPKDTLMISRTPEVTCVVVDVSHEDPEVKFNWYVDGVEVHNAKTKPREEQYNST | |
| | FPAVLQSSGLYSLSSVVTVPSSSLGTQTYICNV | YRVVSVLTVLHQDWLNGKEYKCKVSNKALPAPIEKTISKAKGQPREPQVYTLPPSREEM | |
| | NHKPSNTKVDKKVEPKSCDKTHTCPPCPAPEaa | TKNQVSLWCLVKGFYPSDIAVEWESNGQPENNYKTTPPVLDSDGSFFLYSKLTVDKSRW | |
| | GGPSVFLFPPKPKDTLMISRTPEVTCVVVDVSH | QQGNVFSCSVMHEALHNHYTQKSLSLSPGK (SEQ ID NO: 56) | |
| | EDPEVKFNWYVDGVEVHNAKTKPREEQYNSTYR | | |
| | VVSVLTVLHQDWLNGKEYKCKVSNKALPAPIEK | | |
| | TISKAKGQPREPQVYTLPPSREEMTKNQVSLSC | | |
| | AVKGFYPSDIAVEWESNGQPENNYKTTPPVLDS | | |
| | DGSFFLVSKLTVDKSRWQQGNVFSCSVMHEALH | | |
| | NRFTQKSLSLSPGGGGGSGGGGSGGGGSGGGGS | | |
| | GGGGSGGGGSAAEPVEDNsINFVAMKFIDNTLY | | |
| | FIAEDDENIEQDYFGKLESKLSVIRNLNDQVLF | | |
| | IDQGNRPLFEDMTDSDSRDNAPRTIFIISKYGD | | |
| | SQPRGLAVTISVKSEKISTLSSENKIISFKEMN | | |
| | PPDNIKDTKSDIIFFQRSVPGHDNKVQFESSSY | | |
| | EGYFLASEKERDLFKLILKKEDELGDRSIMFTV | | |
| | QNED (SEQ ID NO: 17) | | |
| FUSE-756 | (SEQ ID NO: 1) | GGGGSGGGGSGGGGSGGGGSGGGGSGGGGSGIEQDYFGKLESKLSVIRNLNDQVLFIDQ | |
| | | GNRPLFEDMTDSDVRDNAPRTIFIISMYKDSQPRGMAVTISVKVEKISTLSVENKIISF | |
| | | KEMNPPDNIKDTKSDIIFFQRSVPGHDNKMQFESSSYEGYFLAVEKERDLFKLILKKED | |
| | | ELGDRSIMFTVQNEDEPKSSDKTHTCPPCPAPELLGGPSVFLFPPKPKDTLMISRTPEV | |
| | | TCVVVDVSHEDPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLWCLVKGFYPSDI | |
| | | AVEWESNGQPENNYKTTPPVLDSDGSFFLYSKLTVDKSRWQQGNVFSCSVMHEALHNHY | |
| | | TQKSLSLSPGK (SEQ ID NO: 57) | |
| FUSE-757 | (SEQ ID NO: 1) | GGGGSGGGGSGIEQDYFGKLESKLSVIRNLNDQVLFIDQGNRPLFEDMTDSDVRDNAPR | |
| | | TIFIISMYKDSQPRGMAVTISVKVEKISTLSVENKIISFKEMNPPDNIKDTKSDIIFFQ | |
| | | RSVPGHDNKMQFESSSYEGYFLAVEKERDLFKLILKKEDELGDRSIMFTVQNEDEPKSS | |
| | | DKTHTCPPCPAPELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVSNKALPAPIEKTISK | |
| | | AKGQPREPQVYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK (SEQ ID | |
| | | NO: 58) | |
| FUSE-758 | (SEQ ID NO: 1) | LQARVVGGGGTIEQDYFGKLESKLSVIRNLNDQVLFIDQGNRPLFEDMTDSDVRDNAPR | |
| | | TIFIISMYKDSQPRGMAVTISVKVEKISTLSVENKIISFKEMNPPDNIKDTKSDIIFFQ | |
| | | RSVPGHDNKMQFESSSYEGYFLAVEKERDLFKLILKKEDELGDRSIMFTVQNEDEPKSS | |
| | | DKTHTCPPCPAPELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVSNKALPAPIEKTISK | |
| | | AKGQPREPQVYTLPPSREEMTKNQVSLWCLVKGFYPSDIAVEWESNGQPENNYKTTPPV | |
| | | LDSDGSFFLYSKLTVDKSRWQQGNVFSCSVMHEALHNHYTQKSLSLSPGK (SEQ ID | |
| | | NO: 59) | |
| FUSE-775 | (SEQ ID NO: 1) | EVQLVESGGGLVQAGGSLRLSCAASGRTESSYAMGWFRQAPGKEREFVVAINWSSGSTY | |
| | | YADSVKGRFTISRDNAKNTMYLQMsSLKPEDTAVYYCAAGYQINSGNYNFKDYEYDYWG | |
| | | QGTQVTVSSEPKSSDKTHTCPPCPAPELLGGPSVFLFPPKPKDTLMISRTPEVTCVVVD | |
| | | VSHEDPEVKFNWYVDGVEVHNAKTKPREEQYNSTYRVVSVLTVLHQDWLNGKEYKCKVS | |
| | | NKALPAPIEKTISKAKGQPREPQVYTLPPSREEMTKNQVSLWCLVKGFYPSDIAVEWES | |
| | | NGQPENNYKTTPPVLDSDGSFFLYSKLTVDKSRWQQGNVFSCSVMHEALHNHYTQKSLS | |
| | | LSPGKAAEPVEDNVINFVAMKFIDNTLYFIAEnDENGGGSYFGKLESKLSVIRNLNDQV | |
| | | LFIDQGNRPLFEDMTDSDVRDNAPRTIFIISKYGDSQPRGLAVTISVKVEKISTLSVEN | |
| | | KIISFKEMNPPDNIKDTKSDIIFFQRSVPGHDNKVQFESSSYEGYFLAVEKERDLEKLI | |
| | | LKKEDELGDRSIMETVQNED (SEQ ID NO: 60) | |
| FUSE-782 | QVQLVESGGGVVQPGRSLRLDCKASGITFSNSG | QVQLVESGGGVVQPGRSLRLDCKASGITFSNSGMHWVRQAPGKGLEWVAVIWYDGSKRY | SEQ |
| | MHWVRQAPGKGLEWVAVIWYDGSKRYYADSVKG | YADSVKGRFTISRDNSKNTLFLQMsSLRAEDTAVYYCATNDDYWGQGTLVTVSSASTKG | ID |
| | RFTISRDNSKNTLFLQMsSLRAEDTAVYYCATN | PSVFPLAPSSKSTSGGTAALGCLVKDYFPEPVTVSWNSGALTSGVHTFPAVLQSSGLYS | NO: 75 |
| | DDYWGQGTLVTVSSASTKGPSVFPLAPSSKSTS | LSSVVTVPSSSLGTQTYICNVNHKPSNTKVDKKVEPKSCDKTHTCPPCPAPEaaGGPSV | |
| | GGTAALGCLVKDYFPEPVTVSWNSGALTSGVHT | FLFPPKPKDTLMISRTPEVTCVVVDVSHEDPEVKFNWYVDGVEVHNAKTKPREEQYNST | |
| | FPAVLQSSGLYSLSSVVTVPSSSLGTQTYICNV | YRVVSVLTVLHQDWLNGKEYKCKVSNKALPAPIEKTISKAKGQPREPQVYTLPPSREEM | |
| | NHKPSNTKVDKKVEPKSCDKTHTCPPCPAPEaa | TKNQVSLWCLVKGFYPSDIAVEWESNGQPENNYKTTPPVLDSDGSFFLYSKLTVDKSRW | |
| | GGPSVFLFPPKPKDTLMISRTPEVTCVVVDVSH | QQGNVFSCSVMHEALHNHYTQKSLSLSPGGGGGSGGGGSGGGGSGGGGSAAEPVEDNsI | |
| | EDPEVKFNWYVDGVEVHNAKTKPREEQYNSTYR | NFVAMKFIDNTLYFIAEnDENgggsYFGKLESKLSVIRNLNDQVLFIDQGNRPLFEDMT | |
| | VVSVLTVLHQDWLNGKEYKCKVSNKALPAPIEK | DSDSRDNAPRTIFIISKYGDSQPRGLAVTISVKSEKISTLSSENKIISFKEMNPPDNIK | |
| | TISKAKGQPREPQVYTLPPSREEMTKNQVSLSC | DTKSDIIFFQRSVPGHDNKVQFESSSYEGYFLASEKERDLFKLILKKEDELGDRSIMFT | |
| | AVKGFYPSDIAVEWESNGQPENNYKTTPPVLDS | VQNED (SEQ ID NO: 61) | |
| | DGSFFLVSKLTVDKSRWQQGNVFSCSVMHEALH | | |
| | NRFTQKSLSLSPGK (SEQ ID NO: 18) | | |
| FUSE-783 | QVQLVESGGGVVQPGRSLRLDCKASGITFSNSG | QVQLVESGGGVVQPGRSLRLDCKASGITFSNSGMHWVRQAPGKGLEWVAVIWYDGSKRY | SEQ |
| | MHWVRQAPGKGLEWVAVIWYDGSKRYYADSVKG | YADSVKGRFTISRDNSKNTLFLQMSSLRAEDTAVYYCATNDDYWGQGTLVTVSSASTKG | ID |
| | RFTISRDNSKNTLFLQMsSLRAEDTAVYYCATN | PSVFPLAPSSKSTSGGTAALGCLVKDYFPEPVTVSWNSGALTSGVHTFPAVLQSSGLYS | NO: 75 |
| | DDYWGQGTLVTVSSASTKGPSVFPLAPSSKSTS | LSSVVTVPSSSLGTQTYICNVNHKPSNTKVDKKVEPKSCDKTHTCPPCPAPEaaGGPSV | |
| | GGTAALGCLVKDYFPEPVTVSWNSGALTSGVHT | FLFPPKPKDTLMISRTPEVTCVVVDVSHEDPEVKFNWYVDGVEVHNAKTKPREEQYNST | |
| | FPAVLQSSGLYSLSSVVTVPSSSLGTQTYICNV | YRVVSVLTVLHQDWLNGKEYKCKVSNKALPAPIEKTISKAKGQPREPQVYTLPPSREEM | |
| | NHKPSNTKVDKKVEPKSCDKTHTCPPCPAPEaa | TKNQVSLWCLVKGFYPSDIAVEWESNGQPENNYKTTPPVLDSDGSFFLYSKLTVDKSRW | |
| | GGPSVFLFPPKPKDTLMISRTPEVTCVVVDVSH | QQGNVFSCSVMHEALHNHYTQKSLSLSPGGGGGSGGGGSGGGGSGGGGSAAEPVEDNVI | |
| | EDPEVKFNWYVDGVEVHNAKTKPREEQYNSTYR | NFVAMKFIDNTLYFIAEnDENgggsYFGKLESKLSVIRNLNDQVLFIDQGNRPLFEDMT | |
| | VVSVLTVLHQDWLNGKEYKCKVSNKALPAPIEK | DSDVRDNAPRTIFIISKYGDSQPRGLAVTISVKVEKISTLSVENKIISFKEMNPPDNIK | |
| | TISKAKGQPREPQVYTLPPSREEMTKNQVSLSC | DTKSDIIFFQRSVPGHDNKVQFESSSYEGYFLAVEKERDLFKLILKKEDELGDRSIMFT | |
| | AVKGFYPSDIAVEWESNGQPENNYKTTPPVLDS | VQNED (SEQ ID NO: 62) | |
| | DGSFFLVSKLTVDKSRWQQGNVFSCSVMHEALH | | |
| | NRFTQKSLSLSPGK (SEQ ID NO: 18) | | |
| FUSE-827 | EPKSSDKTHTCPPCPAPEAAGGPSVFLFPPKPK | EPKSSDKTHTCPPCPAPEaaGGPSVFLFPPKPKDTLMISRTPEVTCVVVDVSHEDPEVK | |
| | DTLMISRTPEVTCVVVDVSHEDPEVKFNWYVDG | FNWYVDGVEVHNAKTKPREEQYNSTYRVVSVLTVLHQDWLNGKEYKCKVSNKALPAPIE | |
| | VEVHNAKTKPREEQYNSTYRVVSVLTVLHQDWL | KTISKAKGQPREPQVYTLPPSREEMTKNQVSLWCLVKGFYPSDIAVEWESNGQPENNYK | |
| | NGKEYKCKVSNKALPAPIEKTISKAKGQPREPQ | TTPPVLDSDGSFFLYSKLTVDKSRWQQGNVFSCSVMHEALHNHYTQKSLSLSPGSGGGG | |
| | VYTLPPSREEMTKNQVSLSCAVKGFYPSDIAVE | AAEPVEDNSINFVAMKFIDNTLYFIAEnDENgggsYFGKLESKLSVIRNLNDQVLFIDQ | |
| | WESNGQPENNYKTTPPVLDSDGSFFLVSKLTVD | GNRPLFEDMTDSDSRDNAPRTIFIISKYGDSQPRGLAVTISVKSEKISTLSSENKIISF | |
| | KSRWQQGNVFSCSVMHEALHNRFTQKSLSLSPG | KEMNPPDNIKDTKSDIIFFQRSVPGHDNKVQFESSSYEGYFLASEKERDLFKLILKKED | |
| | K (SEQ ID NO: 19) | ELGDRSIMFTVQNED (SEQ ID NO: 63) | |
| FUSE-832 | EPKSSDKTHTCPPCPAPEAAGGPSVFLFPPKPK | YFGKLESKLSVIRNLNDQVLFIDQGNRPLFEDMTDSDVRDNAPRTIFIISKYGDSQPRG | |
| | DTLMISRTPEVTCVVVDVSHEDPEVKFNWYVDG | LAVTISVKVEKISTLSVENKIISFKEMNPPDNIKDTKSDIIFFQRSVPGHDNKVQFESS | |
| | VEVHNAKTKPREEQYNSTYRVVSVLTVLHQDWL | SYEGYFLAVEKERDLFKLILKKEDELGDRSIMFTVQNEDEPKSSDKTHTCPPCPAPEAA | |
| | NGKEYKCKVSNKALPAPIEKTISKAKGQPREPQ | GGPSVFLFPPKPKDTLMISRTPEVTCVVVDVSHEDPEVKFNWYVDGVEVHNAKTKPREE | |
| | VYTLPPSREEMTKNQVSLSCAVKGFYPSDIAVE | QYNSTYRVVSVLTVLHQDWLNGKEYKCKVSNKALPAPIEKTISKAKGQPREPQVYTLPP | |
| | WESNGQPENNYKTTPPVLDSDGSFFLVSKLTVD | SREEMTKNQVSLWCLVKGFYPSDIAVEWESNGQPENNYKTTPPVLDSDGSFFLYSKLTV | |
| | KSRWQQGNVFSCSVMHEALHNRFTQKSLSLSPG | DKSRWQQGNVFSCSVMHEALHNHYTQKSLSLSPGK (SEQ ID NO: 276) | |
| | K (SEQ ID NO: 260) | | |
| FUSE-874 | (SEQ ID NO: 1) | SYFGKLESKLSVIRNLNDQVLFIDQGNRPLFEDMTDSDVRDNAPRTIFIISMYKDSQPR | |
| | | GMAVTISVKVEKISTLSVENKIISFKEMNPPDNIKDTKSDIIFFQRSVPGHDNKMQFES | |
| | | SSYEGYFLAVEKERDLFKLILKKEDELGDRSIMFTVQNEDEPKSSDKTHTCPPCPAPEL | |
| | | LGGPSVFLFPPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQPREPQVYTLP | |
| | | PSREEMTKNQVSLWCLVKGFYPSDIAVEWESNGQPENNYKTTPPVLDSDGSFFLYSKLT | |
| | | VDKSRWQQGNVFSCSVMHEALHNHYTQKSLSLSPGK (SEQ ID NO: 64) | |
| FUSE-875 | (SEQ ID NO: 1) | ggggsYFGKLESKLSVIRNLNDQVLFIDQGNRPLFEDMTDSDVRDNAPRTIFIISMYKD | |
| | | SQPRGMAVTISVKVEKISTLSVENKIISFKEMNPPDNIKDTKSDIIFFQRSVPGHDNKM | |
| | | QFESSSYEGYFLAVEKERDLFKLILKKEDELGDRSIMETVQNEDEPKSSDKTHTCPPCP | |
| | | APELLGGPSVFLFPPKPKDTLMISRTPEVTCVVVDVSHEDPEVKFNWYVDGVEVHNAKT | |
| | | KPREEQYNSTYRVVSVLTVLHQDWLNGKEYKCKVSNKALPAPIEKTISKAKGQPREPQV | |
| | | YTLPPSREEMTKNQVSLWCLVKGFYPSDIAVEWESNGQPENNYKTTPPVLDSDGSFFLY | |
| | | SKLTVDKSRWQQGNVFSCSVMHEALHNHYTQKSLSLSPGK (SEQ ID NO: 65) | |
| FUSE-876 | (SEQ ID NO: 1) | ggggsggggsYFGKLESKLSVIRNLNDQVLFIDQGNRPLFEDMTDSDVRDNAPRTIFII | |
| | | SMYKDSQPRGMAVTISVKVEKISTLSVENKIISFKEMNPPDNIKDTKSDIIFFQRSVPG | |
| | | HDNKMQFESSSYEGYFLAVEKERDLFKLILKKEDELGDRSIMFTVQNEDEPKSSDKTHT | |
| | | CPPCPAPELLGGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIEKTISKAKGQP | |
| | | REPQVYTLPPSREEMTKNQVSLWCLVKGFYPSDIAVEWESNGQPENNYKTTPPVLDSDG | |
| | | SFFLYSKLTVDKSRWQQGNVFSCSVMHEALHNHYTQKSLSLSPGK (SEQ ID | |
| | | NO: 66) | |
| FUSE-882 | EVQLVESGGGLVQPGGSLRLSCAASGFTFSDSW | AAEPVEDNVINFVAMKFIDNTLYFIAEnDENgggsYFGKLESKLSVIRNLNDQVLFIDQ | SEQ |
| | IHWVRQAPGKGLEWVAWISPYGGSTYYADSVKG | GNRPLFEDMTDSDVRDNAPRTIFIISKYGDSQPRGLAVTISVKVEKISTLSVENKIISF | ID |
| | RFTISADTSKNTAYLQMsSLRAEDTAVYYCARR | KEMNPPDNIKDTKSDIIFFQRSVPGHDNKVQFESSSYEGYFLAVEKERDLFKLILKKED | NO: 280 |
| | HWPGGFDYWGQGTLVTVSSASTKGPSVFPLAPS | ELGDRSIMFTVQNEDGGGGSGGGGSDKTHTCPPCPAPELLGGPSVFLFPPKPKDTLMIS | |
| | SKSTSGGTAALGCLVKDYFPEPVTVSWNSGALT | RTPEVTCVVVDVSHEDPEVKFNWYVDGVEVHNAKTKPREEQYNSTYRVVSVLTVLHQDW | |
| | SGVHTFPAVLQSSGLYSLSSVVTVPSSSLGTQT | LNGKEYKCKVSNKALPAPIEKTISKAKGQPREPQVYTLPPSREEMTKNQVSLWCLVKGF | |
| | YICNVNHKPSNTKVDKKVEPKSCDKTHTCPPCP | YPSDIAVEWESNGQPENNYKTTPPVLDSDGSFFLYSKLTVDKSRWQQGNVFSCSVMHEA | |
| | APELLGGPSVFLFPPKPKDTLMISRTPEVTCVV | LHNHYTQKSLSLSPGK (SEQ ID NO: 277) | |
| | VDVSHEDPEVKFNWYVDGVEVHNAKTKPREEQY | | |
| | NSTYRVVSVLTVLHQDWLNGKEYKCKVSNKALP | | |
| | APIEKTISKAKGQPREPQVYTLPPSREEMTKNQ | | |
| | VSLSCAVKGFYPSDIAVEWESNGQPENNYKTTP | | |
| | PVLDSDGSFFLVSKLTVDKSRWQQGNVFSCSVM | | |
| | HEALHNRFTQKSLSLSPGK (SEQ ID | | |
| | NO: 261) | | |
| FUSE-887 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNtINFVAMKFIDNTLYFIAEDDENLESDYFGKLESKLSVIRNLNDQVLFIDQGNRP | |
| | | LFEDMTDSDSRDNAPRTIFIISMYKDSQPRGMAVTISVKSEKISTLSSENKIISFKEMN | |
| | | PPDNIKDTKSDIIFFQRSVPGHDNKMQFESSSYEGYFLASEKERDLFKLILKKEDELGD | |
| | | RSIMFTVQNED (SEQ ID NO: 67) | |
| FUSE-888 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNqINFVAMKFIDNTLYFIAEDDENLESDYFGKLESKLSVIRNLNDQVLFIDQGNRP | |
| | | LFEDMTDSDSRDNAPRTIFIISMYKDSQPRGMAVTISVKSEKISTLSSENKIISFKEMN | |
| | | PPDNIKDTKSDIIFFQRSVPGHDNKMQFESSSYEGYFLASEKERDLFKLILKKEDELGD | |
| | | RSIMFTVQNED (SEQ ID NO: 68) | |
| FUSE-889 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNdINFVAMKFIDNTLYFIAEDDENLESDYFGKLESKLSVIRNLNDQVLFIDQGNRP | |
| | | LFEDMTDSDaRDNAPRTIFIISMYKDSQPRGMAVTISVKaEKISTLSaENKIISFKEMN | |
| | | PPDNIKDTKSDIIFFQRSVPGHDNKMQFESSSYEGYFLAaEKERDLEKLILKKEDELGD | |
| | | RSIMFTVQNED (SEQ ID NO: 69) | |
| FUSE-890 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNIINFVAMKFIDNTLYFIAEDDENLESDYFGKLESKLSVIRNLNDQVLFIDQGNRP | |
| | | LFEDMTDSDaRDNAPRTIFIISMYKDSQPRGMAVTISVKaEKISTLSaENKIISFKEMN | |
| | | PPDNIKDTKSDIIFFQRSVPGHDNKMQFESSSYEGYFLAaEKERDLEKLILKKEDELGD | |
| | | RSIMFTVQNED (SEQ ID NO: 70) | |
| FUSE-891 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNIINFVAMKFIDNTLYFIAEDDENLESDYGKLESKLSVIRNLNDQVLFIDQGNRP | |
| | | LFEDMTDSDVRDNAPRTIFIISMYKDSQPRGMAVTISVKVEKISTLSVENKIISFKEMN | |
| | | PPDNIKDTKSDIIFFQRSVPGHDNKMQFESSSYEGYFLAVEKERDLEKLILKKEDELGD | |
| | | RSIMFTVQNED (SEQ ID NO: 71) | |
| FUSE-892 | (SEQ ID NO: 1) | EPKSSDKTHTCPPCPAPELLGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVSNKALPAPIE | |
| | | KTISKAKGQPREPQVYTLPPSREEMTKNQVSLWCLVKGFYPSDIAVEWESNGQPENNYK | |
| | | TTPPVLDSDGSFFLYSKLTVDKSRWQQGNVFSCSVMHEALHNHYTQKSLSLSPGKAAEP | |
| | | VEDNhINFVAMKFIDNTLYFIAEDDENLESDYFGKLESKLSVIRNLNDQVLFIDQGNRP | |
| | | LFEDMTDSDVRDNAPRTIFIISMYKDSQPRGMAVTISVKVEKISTLSVENKIISFKEMN | |
| | | PPDNIKDTKSDIIFFQRSVPGHDNKMQFESSSYEGYFLAVEKERDLEKLILKKEDELGD | |
| | | RSIMFTVQNED (SEQ ID NO: 72) | |
| FUSE-895 | (SEQ ID NO: 261) | AAEPVEDNVINFVAMKFIDNTLYFIAEnDENqggsYFGKLESKLSVIRNLNDQVLFIDQ | SEQ |
| | | GNRPLFEDMTDSDVRDNAPRTIFIISKYGDSQPRGLAVTISVKVEKISTLSVENKIISF | ID |
| | | KEMNPPDNIKDTKSDIIFFQRSVPGHDNKVQFESSSYEGYFLAVEKERDLFKLILKKED | NO: 280 |
| | | ELGDRSIMFTVQNEDGGGGSGGGGSDKTHTCPPCPAPELLGGPSVFLFPPKPKDTLMIS | |
| | | RTPEVTCVVVDVSHEDPEVKFNWYVDGVEVHNAKTKPREEQYNSTYRVVSVLTVLHQDW | |
| | | LNGKEYKCKVSNKALPAPIEKTISKAKGQPREPQVYTLPPSREEMTKNQVSLWCLVKGF | |
| | | YPSDIAVEWESNGQPENNYKTTPPVLDSDGSFFLYSKLTVDKSRWQQGNVFSCSVMHEA | |
| | | LHNHYTQKSLSLSPGK (SEQ ID NO: 278) | |
| FUSE-952 | SEQ ID NO: 260 | EVQLQESGPGLVKPSQSLSLTCSVTGYSITSSYRWNWIRKFPGNRLEWMGYINSAGISN | SEQ |
| | | YNPSLKRRISITRDTSKNQFFLQVNSVTTEDAATYYCARSDNMGTTPFTYWGQGTLVTV | ID |
| | | SSASTKGPSVFPLAPSSKSTSGGTAALGCLVKDYFPEPVTVSWNSGALTSGVHTFPAVL | NO: 281 |
| | | QSSGLYSLSSVVTVPSSSLGTQTYICNVNHKPSNTKVDKKVEPKSCDKTHTCPPCPAPE | |
| | | aaGGPSVFLFPPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDSDGSFFLYSKL | |
| | | TVDKSRWQQGNVFSCSVMHEALHNHYTQKSLSLSPGK (SEQ ID NO: 279) | |
| FUSE-1015 | AAEPVEDNVINFVAMKFIDNTLYFIAEnDENgg | SEQ ID NO: 279 | SEQ |
| | DMTDSDVRDNAPRTIFIISKYGDSQPRGLAVTI | | ID |
| | gSYFGKLESKLSVIRNLNDQVLFIDQGNRPLFE | | NO: 281 |
| | SVKVEKISTLSVENKIISFKEMNPPDNIKDTKS | | |
| | DIIFFQRSVPGHDNKVQFESSSYEGYFLAVEKE | | |
| | RDLFKLILKKEDELGDRSIMFTVQNEDEPKSSD | | |
| | KTHTCPPCPAPEaaGGPSVFLFPPKPKDTLMIS | | |
| | RTPEVTCVVVDVSHEDPEVKFNWYVDGVEVHNA | | |
| | KTKPREEQYNSTYRVVSVLTVLHQDWINGKEYK | | |
| | CKVSNKALPAPIEKTISKAKGQPREPQVYTLPP | | |
| | SREEMTKNQVSLSCAVKGFYPSDIAVEWESNGQ | | |
| | PENNYKTTPPVLDSDGSFFLVSKLTVDKSRWQQ | | |
| | GNVFSCSVMHEALHNRFTQKSLSLSPGK (SEQ | | |
| | ID NO: 262) | | |
| FUSE-1017 | SEQ ID NO: 18 | SEQ ID NO: 276 | SEQ |
| | | | ID |
| | | | NO: 75 |
| FUSE-1041 | SEQ ID NO: 260 | SEQ ID NO: 56 | SEQ |
| | | | ID |
| | | | NO: 75 |
| FUSE-1042 | SEQ ID NO: 262 | SEQ ID NO: 56 | SEQ |
| | | | ID |
| | | | NO: 75 |
| FUSE-1044 | AAEPVEDNVINFVAMKFIDNTLYFIAEnDENgg | SEQ ID NO: 56 | SEQ |
| | gSYFGKLKSKLSVIRNLNDQVLFIDQGNRPLFE | | ID |
| | DMTDSDVRDNAPRTIFIIShYgDSQPRGMAVTI | | NO: 75 |
| | SVKVEKISTLSVENKIISFKEMNPPDNIKDTKS | | |
| | DIIFFQRSVPGHDNKMQFESSSYEGYFLAVEKE | | |
| | RDLFKLILKKEDELGDRSIMFTVQNEDEPKSSD | | |
| | KTHTCPPCPAPEaaGGPSVFLFPPKPKDTLMIS | | |
| | RTPEVTCVVVDVSHEDPEVKFNWYVDGVEVHNA | | |
| | KTKPREEQYNSTYRVVSVLTVLHQDWLNGKEYK | | |
| | CKVSNKALPAPIEKTISKAKGQPREPQVYTLPP | | |
| | SREEMTKNQVSLSCAVKGFYPSDIAVEWESNGQ | | |
| | PENNYKTTPPVLDSDGSFFLVSKLTVDKSRWQQ | | |
| | GNVFSCSVMHEALHNRFTQKSLSLSPGK (SEQ | | |
| | ID NO: 263) | | |
| FUSE-1109 | YFGKLQSKLSVIRNLNDQVLFIDQGNRPLFEDM | SEQ ID NO: 56 | SEQ |
| | KVEKISTLSVENKIISFKEMNPPDNIKDTKSDI | | ID |
| | TDSDVRDNAPRTIFIISMYDDSQPRGKAVTISV | | |
| | IFFQRSVPGHDNKMQFESSSYEGYFLAVEKERD | | NO: 75 |
| | LFKLILKKEDELGDRSIMFTVQNEDEPKSSDKT | | |
| | HTCPPCPAPEAAGGPSVFLFPPKPKDTLMISRT | | |
| | PEVTCVVVDVSHEDPEVKFNWYVDGVEVHNAKT | | |
| | KPREEQYNSTYRVVSVLTVLHQDWLNGKEYKCK | | |
| | VSNKALPAPIEKTISKAKGQPREPQVYTLPPSR | | |
| | EEMTKNQVSLSCAVKGFYPSDIAVEWESNGQPE | | |
| | NNYKTTPPVLDSDGSFFLVSKLTVDKSRWQQGN | | |
| | VFSCSVMHEALHNRFTQKSLSLSPG (SEQ ID | | |
| | NO: 264) | | |
| FUSE-1110 | YFGKLQSKLSVIRNLNDQVLKIDQGNRPLFEDM | SEQ ID NO: 56 | SEQ |
| | TDSDVRDNAPRTIFIISMYDDSQPRGKAVTISV | | ID |
| | KVEKISTLSVENKIISFKEMNPPDNIKDTKSDI | | NO: 75 |
| | IFFQRSVPGHDNKMQFESSSYEGYFLAVEKERD | | |
| | LFKLILKKEDELGDRSIMFTVQNEDEPKSSDKT | | |
| | HTCPPCPAPEAAGGPSVFLFPPKPKDTLMISRT | | |
| | PEVTCVVVDVSHEDPEVKFNWYVDGVEVHNAKT | | |
| | KPREEQYNSTYRVVSVLTVLHQDWLNGKEYKCK | | |
| | VSNKALPAPIEKTISKAKGQPREPQVYTLPPSR | | |
| | EEMTKNQVSLSCAVKGFYPSDIAVEWESNGQPE | | |
| | NNYKTTPPVLDSDGSFFLVSKLTVDKSRWQQGN | | |
| | VFSCSVMHEALHNRFTQKSLSLSPG (SEQ ID | | |
| | NO: 265) | | |
| FUSE-1111 | YFGKLQSKLSVIRNLNDQVLFIDQGNRPLFEDM | SEQ ID NO: 56 | SEQ |
| | TDSDVRDNAPRTIFIISMYDDSQPRGKAVTISV | | ID |
| | KVEKISTLSVENKIISFKEMNPPDNIKDTKSDI | | NO: 75 |
| | IFFQRSVPGHDNEMQFESSSYEGYFLAVEKERD | | |
| | LFKLILKKEDELGDRSIMFTVQNEDEPKSSDKT | | |
| | HTCPPCPAPEAAGGPSVFLFPPKPKDTLMISRT | | |
| | PEVTCVVVDVSHEDPEVKFNWYVDGVEVHNAKT | | |
| | KPREEQYNSTYRVVSVLTVLHQDWLNGKEYKCK | | |
| | VSNKALPAPIEKTISKAKGQPREPQVYTLPPSR | | |
| | EEMTKNQVSLSCAVKGFYPSDIAVEWESNGQPE | | |
| | NNYKTTPPVLDSDGSFFLVSKLTVDKSRWQQGN | | |
| | VFSCSVMHEALHNRFTQKSLSLSPG (SEQ ID | | |
| | NO: 266) | | |
| FUSE-1112 | YFGKLKSKLSVIRNLNDQVLFIDQGNRPLFEDM | SEQ ID NO: 56 | SEQ |
| | TDSDVRDNAPRTIFIISMYDDSQPRGRAVTISV | | ID |
| | KVEKISTLSVENKIISFKEMNPPDNIKDTKSDI | | NO: 75 |
| | IFFQRSVPGHDNKMQFESSSYEGYFLAVEKERD | | |
| | LFKLILKKEDELGDRSIMFTVQNEDEPKSSDKT | | |
| | HTCPPCPAPEAAGGPSVFLFPPKPKDTLMISRT | | |
| | PEVTCVVVDVSHEDPEVKFNWYVDGVEVHNAKT | | |
| | KPREEQYNSTYRVVSVLTVLHQDWLNGKEYKCK | | |
| | VSNKALPAPIEKTISKAKGQPREPQVYTLPPSR | | |
| | EEMTKNQVSLSCAVKGFYPSDIAVEWESNGQPE | | |
| | NNYKTTPPVLDSDGSFFLVSKLTVDKSRWQQGN | | |
| | VFSCSVMHEALHNRFTQKSLSLSPG (SEQ ID | | |
| | NO: 274) | | |
| FUSE-1113 | GGGGSYFGKLESKLSVIRNLNDQVLFIDQGNRP | SEQ ID NO: 279 | SEQ |
| | LFEDMTDSDVRDNAPRTIFIISKYGDSQPRGLA | | ID |
| | VTISVKVEKISTLSVENKIISFKEMNPPDNIKD | | NO: 281 |
| | TKSDIIFFQRSVPGHDNKVQFESSSYEGYFLAV | | |
| | EKERDLFKLILKKEDELGDRSIMFTVQNEDEPK | | |
| | SSDKTHTCPPCPAPEAAGGPSVFLFPPKPKDTL | | |
| | MISRTPEVTCVVVDVSHEDPEVKFNWYVDGVEV | | |
| | HNAKTKPREEQYNSTYRVVSVLTVLHQDWLNGK | | |
| | EYKCKVSNKALPAPIEKTISKAKGQPREPQVYT | | |
| | LPPSREEMTKNQVSLSCAVKGFYPSDIAVEWES | | |
| | NGQPENNYKTTPPVLDSDGSFFLVSKLTVDKSR | | |
| | WQQGNVFSCSVMHEALHNRFTQKSLSLSPGK | | |
| | (SEQ ID NO: 268) | | |
| FUSE-1116 | KVEKISTLSVENKIISFKEMNPPDNIKDTKSDI | SEQ ID NO: 56 | SEQ |
| | YFGKLESKLSVIRNLNDQVLFIDQGNRPLFEDM | | ID |
| | TDSDVRDNAPRTIFIISKYGDSQPRGLAVTISV | | NO: 75 |
| | IFFQRSVPGHRNKVQFESSSYEGYFLAVEKERD | | |
| | LFKLILKKEDELGDRSIMFTVQNEDEPKSSDKT | | |
| | HTCPPCPAPEAAGGPSVFLFPPKPKDTLMISRT | | |
| | PEVTCVVVDVSHEDPEVKFNWYVDGVEVHNAKT | | |
| | KPREEQYNSTYRVVSVLTVLHQDWLNGKEYKCK | | |
| | VSNKALPAPIEKTISKAKGQPREPQVYTLPPSR | | |
| | EEMTKNQVSLSCAVKGFYPSDIAVEWESNGQPE | | |
| | NNYKTTPPVLDSDGSFFLVSKLTVDKSRWQQGN | | |
| | VFSCSVMHEALHNRFTQKSLSLSPGK (SEQ | | |
| | ID NO: 269) | | |
| FUSE-1176 | EVQLQESGPGLVKPSQSLSLTCSVTGYSITSSY | — | SEQ |
| | RWNWIRKFPGNRLEWMGYINSAGISNYNPSLKR | | ID |
| | RISITRDTSKNQFFLQVNSVTTEDAATYYCARS | | NO: 282 |
| | DNMGTTPFTYWGQGTLVTVSSASTKGPSVEPLA | | |
| | PSSKSTSGGTAALGCLVKDYFPEPVTVSWNSGA | | |
| | LTSGVHTFPAVLQSSGLYSLSSVVTVPSSSLGT | | |
| | QTYICNVNHKPSNTKVDKKVEPKSCDKTHTCPP | | |
| | CPAPEAAGGPSVFLFPPKPKDTLMISRTPEVTC | | |
| | VVVDVSHEDPEVKFNWYVDGVEVHNAKTKPREE | | |
| | QYNSTYRVVSVLTVLHQDWLNGKEYKCKVSNKA | | |
| | LPAPIEKTISKAKGQPREPQVYTLPPSREEMTK | | |
| | NQVSLTCLVKGFYPSDIAVEWESNGQPENNYKT | | |
| | TPPVLDSDGSFFLYSKLTVDKSRWQQGNVFSCS | | |
| | VMHEALHNHYTQKSLSLSPGK (SEQ ID | | |
| | NO: 270) | | |
| FUSE- | GGGGSYFGKLqSKLSVIRNLNDQVLFIDQGNRP | SEQ ID NO: 56 | SEQ |
| 1177 | LFEDMTDSDVRDNAPRTIFIISMYdDSQPRGKA | | ID |
| | VTISVKVEKISTLSVENKIISFKEMNPPDNIKD | | NO: 75 |
| | TKSDIIFFQRSVPGHDNKMQFESSSYEGYFLAV | | |
| | EKERDLFKLILKKEDELGDRSIMFTVQNEDEPK | | |
| | SSDKTHTCPPCPAPEaaGGPSVFLFPPKPKDTL | | |
| | MISRTPEVTCVVVDVSHEDPEVKFNWYVDGVEV | | |
| | HNAKTKPREEQYNSTYRVVSVLTVLHQDWLNGK | | |
| | EYKCKVSNKALPAPIEKTISKAKGQPREPQVYT | | |
| | LPPSREEMTKNQVSLSCAVKGFYPSDIAVEWES | | |
| | NGQPENNYKTTPPVLDSDGSFFLVSKLTVDKSR | | |
| | WQQGNVFSCSVMHEALHNRFTQKSLSLSPG | | |
| | (SEQ ID NO: 271) | | |

Polypeptide 3 sequences

DILLTQSPVILSVSPGERVSFSCRASQSIGTNIHWYQQRINGSPRLLIKYASESISGIPSRFSGSGSGTDFTLSINSVESEDIADYYCQQNNNW
PTTFGAGTKLELKRTVAAPSVEIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC (SEQ ID NO: 73)
DIQMTQSPSSLSASVGDRVTITCRASQDVNTAVAWYQQKPGKAPKLLIYSASFLYSGVPSRFSGSRSGTDFTLTISSLQPEDFATYYCQQHYTT
PPTFGQGTKVEIKRTVAAPSVFIFPPSDEQLKSGTASVVCLLNNFYPREAKVQWKVDNALQSGNSQESVTE (SEQ ID NO: 74)
EIVLTQSPATLSLSPGERATLSCRASQSVSSYLAWYQQKPGQAPRLLIYDASNRATGIPARFSGSGSGTDFTLTISSLEPEDFAVYYCQQSSNW
PRTFGQGTKVEIKRTVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC (SEQ ID NO: 75)
DIQMTQSPSSLSASVGDRVTITCRASQDVSTAVAWYQQKPGKAPKLLIYSASFLYSGVPSRFSGSGSGTDFTLTISSLQPEDFATYYCQQYLYH
PATFGQGTKVEIKRTVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC (SEQ ID NO: 280)
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQ
QGLEFPTFGGGTKLELKRTVAAPSVFIFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC (SEQ ID NO: 281)
GGGGSYFGKLESKLSVIRNLNDQVLFIDQGNRPLFEDMTDSDVRDNAPRTIFIISKYGDSQPRGLAVTISVKVEKISTLSVENKIISFKEMNPP
DNIKDTKSDIIFFQRSVPGHDNKVQFESSSYEGYFLAVEKERDLFKLILKKEDELGDRSIMFTVQNEDGGGGSGGGGSGGGGSGGGGSDIVMTQ
GTLPNPVPSGESVSITCRSSKSLLYSDGKTYLNWYLQRPGQSPQLLIYWMSTRASGVSDRFSGSGSGTDFTLKISGVEAEDVGIYYCQQGLEFP
TFGGGTKLELKRTVAAPSVFIFPPSDEQLKSGTASVVCLLNNFYPREAKVQWKVDNALQSGNSQESVTEQDSKDSTYSLSSTLTLSKADYEKHK
VYACEVTHQGLSSPVTKSFNRGEC (SEQ ID NO: 282)

TABLE 1B

| | | | Mature IL18 |
|---|---|---|---|
| | Propeptide | Cleavage | variant |
| FUSE-422 | AAEPVEDNSINFVAMKFIDNTLY | DDDDK (SEQ | YFGKLESKLSVIRNLNDQVLFIDQGNRPLFEDMTDSDSRDNAPRTIFIISKYGDSQPRG |
| | FIAEDDEN (SEQ ID NO: 76) | ID NO: 87) | LAVTISVKSEKISTLSSENKIISFKEMNPPDNIKDTKSDIIFFQRSVPGHDNKVQFESS |
| | | | SYEGYFLASEKERDLFKLILKKEDELGDRSIMFTVQNED (SEQ ID NO: 97) |
| FUSE-423 | AAEPVEDNAINFVAMKFIDNTLY | SEQ ID NO: 87 | YFGKLESKLSVIRNLNDQVLFIDQGNRPLFEDMTDSDARDNAPRTIFIISKYGDSQPRG |
| | FIAEDDEN (SEQ ID NO: 77) | | LAVTISVKAEKISTLSAENKIISFKEMNPPDNIKDTKSDIIFFQRSVPGHDNKVQFESS |
| | | | SYEGYFLAAEKERDLFKLILKKEDELGDRSIMFTVQNED (SEQ ID NO: 98) |
| FUSE-424 | AAEPVEDNVINFVAMKFIDNTLY | SEQ ID NO: 87 | YFGKLESKLSVIRNLNDQVLFIDQGNRPLFEDMTDSDVRDNAPRTIFIISKYGDSQPRG |
| | FIAEDDEN (SEQ ID NO: 78) | | LAVTISVKVEKISTLSVENKIISFKEMNPPDNIKDTKSDIIFFQRSVPGHDNKVQFESS |
| | | | SYEGYFLAVEKERDLFKLILKKEDELGDRSIMFTVQNED (SEQ ID NO: 99) |
| FUSE-441 | | SEQ ID NO: 87 | SEQ ID NO: 99 |
| FUSE-442 | SEQ ID NO: 78 | SEQ ID NO: 87 | YFGKLESKLSVIRNLNDQVLFIDQGNRPLFEDMTDSDVRDNAPRTIFIISMYKDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 100) |
| FUSE-462 | SEQ ID NO: 78 | IEQD (SEQ ID | SEQ ID NO: 99 |
| | | NO: 88) | |
| FUSE-480 | SEQ ID NO: 76 | SEQ ID NO: 87 | YFGKLESKLSVIRNLNDQVLFIDQGNRPLFEDMTDSDSRDNAPRTIFIISMYKDSQPRG |
| | | | MAVTISVKSEKISTLSSENKIISFKEMNPPDNIKDTKSDIIFFQRSVPGHDNKMQFESS |
| | | | SYEGYFLASEKERDLFKLILKKEDELGDRSIMFTVQNED (SEQ ID NO: 101) |
| FUSE-481 | SEQ ID NO: 77 | SEQ ID NO: 87 | YFGKLESKLSVIRNLNDQVLFIDQGNRPLFEDMTDSDARDNAPRTIFIISMYKDSQPRG |
| | | | MAVTISVKAEKISTLSAENKIISFKEMNPPDNIKDTKSDIIFFQRSVPGHDNKMQFESS |
| | | | SYEGYFLAAEKERDLFKLILKKEDELGDRSIMFTVQNED (SEQ ID NO: 102) |
| FUSE-484 | SEQ ID NO: 78 | SEQ ID NO: 87 | YFGKLESKLSVIRNLNDQVLFIDQGNRPLFEDMTDSDVRDNAPRTIFIISKYGDSGARG |
| | | | LAVTISVKVEKISTLSVENKIISFKEMNPPDNIKDTKSDIIFFERDVPGHSGKVQFESS |
| | | | SYEGYFLAVEKERDLFKLILKKEDELGDRSIMFTVQNED (SEQ ID NO: 103) |
| FUSE-485 | SEQ ID NO: 78 | SEQ ID NO: 88 | SEQ ID NO: 100 |
| FUSE-486 | SEQ ID NO: 78 | GPLGVR (SEQ | SEQ ID NO: 100 |
| | | ID NO: 89) | |
| FUSE-487 | SEQ ID NO: 78 | VHMPLGELGP | SEQ ID NO: 100 |
| | | (SEQ ID | |
| | | NO: 90) | |
| FUSE-499 | SEQ ID NO: 78 | LESD (SEQ ID | SEQ ID NO: 100 |
| | | NO: 91) | |
| FUSE-500 | | | SEQ ID NO: 100 |
| FUSE-501 | SEQ ID NO: 78 | SEQ ID NO: 87 | SEQ ID NO: 100 |
| FUSE-502 | SEQ ID NO: 78 | SEQ ID NO: 87 | SEQ ID NO: 99 |
| FUSE-503 | | SEQ ID NO: 87 | SEQ ID NO: 100 |
| FUSE-504 | | SEQ ID NO: 87 | SEQ ID NO: 99 |
| FUSE-505 | | SEQ ID NO: 87 | SEQ ID NO: 100 |
| FUSE-507 | SEQ ID NO: 78 | SEQ ID NO: 87 | SEQ ID NO: 100 |
| FUSE-508 | | SEQ ID NO: 87 | SEQ ID NO: 100 |
| FUSE-509 | SEQ ID NO: 78 | SEQ ID NO: 87 | SEQ ID NO: 100 |
| FUSE-510 | | SEQ ID NO: 87 | SEQ ID NO: 100 |
| FUSE-516 | SEQ ID NO: 78 | SEQ ID NO: 88 | SEQ ID NO: 99 |
| FUSE-517 | SEQ ID NO: 78 | SEQ ID NO: 88 | SEQ ID NO: 99 |
| FUSE-545 | | | SEQ ID NO: 99 |
| FUSE-546 | | | YFGKLKSKLSVIRNLNDQVLFIDQGNRPLFEDMTDSDVRDNAPRTIFIISMYgDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 104) |
| FUSE-547 | | | YFGKLrSKLSVIRNLNDQVLFIDQGNRPLFEDMTDSDVRDNAPRTIFIISMYgDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 105) |
| FUSE-555 | SEQ ID NO: 78 | SEQ ID NO: 88 | SEQ ID NO: 99 |
| FUSE-556 | | | |
| FUSE-557 | | | |
| FUSE-583 | | | YFGKLaSKLSVIRNLNDQVLFIDQGNRPLFEDMTDSDVRDNAPRTIFIISMYaDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 106) |
| FUSE-584 | | | YFGKLhSKLSVIRNLNDQVLFIDQGNRPLFEDMTDSDVRDNAPRTIFIISMYgDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 107) |
| FUSE-585 | | | YFGKLrSKLSVIRNLNDQVLFIDQGNRPLFEDMTDSDVRDNAPRTIFIISMYeDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 108) |
| FUSE-586 | | | YFGKLqSKLSVIRNLNDQVLFIDQGNRPLFEDMTDSDVRDNAPRTIFIISMYaDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 109) |
| FUSE-587 | SEQ ID NO: 78 | SEQ ID NO: 88 | SEQ ID NO: 100 |
| FUSE-599 | | | YFGKLkSKLSVIRNLNDQVLFIDQGNRPLFEDMTDSDVRDNAPRTIFIIShYgDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 110) |
| FUSE-600 | | | YFGKLkSKLSVIRNLNDQVLFIDQGNRPLFEDMTDSDVRDNAPRTIFIISkYgDSQPRG |
| | | | MAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 111) |
| FUSE-601 | | | YFGKLESKLSVIRNLNDQVLFIDQGNRPLFEDMTDSDVRDNAPRTIFIIShYaDSQPRG |
| | | | iAVTISVKVEKISTLSVENKIISFKEMNPPDNIKDTKSDIIFFQRSVPGHDNKIQFESS |
| | | | SYEGYFLAVEKERDLFKLILKKEDELGDRSIMFTVQNED (SEQ ID NO: 112) |
| FUSE-602 | | | YFGKLESKLSVIRNLNDQVLFIDQGNRPLFEDMTDSDVRDNAPRTIFIISKYaDSQPRG |
| | | | iAVTISVKVEKISTLSVENKIISFKEMNPPDNIKDTKSDIIFFQRSVPGHDNKIQFESS |
| | | | SYEGYFLAVEKERDLFKLILKKEDELGDRSIMFTVQNED (SEQ ID NO: 113) |
| FUSE-624 | SEQ ID NO: 78 | KPLGLQARVVGG | SEQ ID NO: 99 |
| | | GGTIEQD (SEQ | |
| | | ID NO: 92) | |
| FUSE-625 | SEQ ID NO: 78 | SEQ ID NO: 92 | SEQ ID NO: 99 |
| FUSE-626 | SEQ ID NO: 78 | | SEQ ID NO: 99 |
| FUSE-627 | SEQ ID NO: 78 | SEQ ID NO: 88 | SEQ ID NO: 99 |
| FUSE-645 | SEQ ID NO: 76 | SEQ ID NO: 88 | SEQ ID NO: 97 |
| FUSE-658 | SEQ ID NO: 78 | IGNRS (SEQ | SEQ ID NO: 99 |
| | | ID NO: 93) | |
| FUSE-659 | SEQ ID NO: 78 | KPLGLQARVV | SEQ ID NO: 99 |
| | | (SEQ ID | |
| | | NO: 94) | |
| FUSE-660 | SEQ ID NO: 78 | IEQDGGSIGNRS | SEQ ID NO: 99 |
| | | GGGGKPLGLQAR | |
| | | VVGGGGTIEQD | |
| | | (SEQ ID | |
| | | NO: 95) | |
| FUSE-669 | | | |
| FUSE-670 | SEQ ID NO: 78 | SEQ ID NO: 88 | SEQ ID NO: 99 |
| FUSE-675 | | | |
| FUSE-676 | SEQ ID NO: 78 | SEQ ID NO: 88 | SEQ ID NO: 99 |
| FUSE-682 | SEQ ID NO: 78 | SEQ ID NO: 88 | SEQ ID NO: 99 |
| FUSE-683 | SEQ ID NO: 78 | SEQ ID NO: 88 | SEQ ID NO: 99 |
| FUSE-684 | SEQ ID NO: 78 | SEQ ID NO: 88 | SEQ ID NO: 99 |
| FUSE-685 | SEQ ID NO: 76 | | SEQ ID NO: 97 |
| FUSE-686 | AAEPVEDNSINFVAMKFIDNTLY | QDIE (SEQ ID | SEQ ID NO: 97 |
| | FIAEnDEN (SEQ ID NO: 79) | NO: 96) | |
| FUSE-691 | | | |
| FUSE-694 | SEQ ID NO: 76 | SEQ ID NO: 88 | SEQ ID NO: 97 |
| FUSE-756 | | SEQ ID NO: 88 | SEQ ID NO: 100 |
| FUSE-757 | | SEQ ID NO: 88 | SEQ ID NO: 100 |
| FUSE-758 | | SEQ ID NO: 88 | SEQ ID NO: 100 |
| FUSE-775 | AAEPVEDNVINFVAMKFIDNTLY | | SEQ ID NO: 99 |
| | FIAEnDEN (SEQ ID NO: 80) | | |
| FUSE-782 | SEQ ID NO: 79 | | SEQ ID NO: 97 |
| FUSE-783 | SEQ ID NO: 80 | | SEQ ID NO: 99 |
| FUSE-827 | SEQ ID NO: 79 | | SEQ ID NO: 97 |
| FUSE-832 | | | SEQ ID NO: 99 |
| FUSE-874 | | | SEQ ID NO: 100 |
| FUSE-875 | | | SEQ ID NO: 100 |
| FUSE-876 | | | SEQ ID NO: 100 |
| FUSE-882 | SEQ ID NO: 80 | | SEQ ID NO: 99 |
| FUSE-887 | AAEPVEDNtINFVAMKFIDNTLY | | SEQ ID NO: 101 |
| | FIAEDDENLESD (SEQ ID | | |
| | NO: 81) | | |
| FUSE-888 | AAEPVEDNqINFVAMKFIDNTLY | | SEQ ID NO: 101 |
| | FIAEDDENLESD (SEQ ID | | |
| | NO: 82) | | |
| FUSE-889 | AAEPVEDNdINFVAMKFIDNTLY | | SEQ ID NO: 102 |
| | FIAEDDENLESD (SEQ ID | | |
| | NO: 83) | | |
| FUSE-890 | AAEPVEDNIINFVAMKFIDNTLY | | SEQ ID NO: 102 |
| | FIAEDDENLESD (SEQ ID | | |
| | NO: 84) | | |
| FUSE-891 | AAEPVEDNIINFVAMKFIDNTLY | | SEQ ID NO: 100 |
| | FIAEDDENLESD (SEQ ID | | |
| | NO: 85) | | |
| FUSE-892 | AAEPVEDNhINFVAMKFIDNTLY | | SEQ ID NO: 100 |
| | FIAEDDENLESD (SEQ ID | | |
| | NO: 86) | | |
| FUSE-895 | SEQ ID NO: 80 | | SEQ ID NO: 99 |
| FUSE-952 | | | |
| FUSE- | SEQ ID NO: 80 | | SEQ ID NO: 99 |
| 1015 | | | |
| FUSE- | SEQ ID NO: 131 | | SEQ ID NO: 99 |
| 1017 | | | |
| FUSE- | | | |
| 1041 | | | |
| FUSE- | SEQ ID NO: 80 | | SEQ ID NO: 99 |
| 1042 | | | |
| FUSE- | SEQ ID NO: 80 | | SEQ ID NO: 110 |
| 1044 | | | |
| FUSE- | | | YFGKLqSKLSVIRNLNDQVLFIDQGNRPLFEDMTDSDVRDNAPRTIFIISMYdDSQPRG |
| 1109 | | | kAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 283) |
| FUSE- | | | YFGKLqSKLSVIRNLNDQVLKIDQGNRPLFEDMTDSDVRDNAPRTIFIISMYdDSQPRG |
| 1110 | | | kAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 284) |
| FUSE- | | | YFGKLqSKLSVIRNLNDQVLFIDQGNRPLFEDMTDSDVRDNAPRTIFIISMYdDSQPRG |
| 1111 | | | kAVTISVKVEKISTLSVENKIISFKEMNPPDNIKDTKSDIIFFQRSVPGHDNeMQFESS |
| | | | SYEGYFLAVEKERDLFKLILKKEDELGDRSIMFTVQNED (SEQ ID NO: 285) |
| FUSE- | | | YFGKLKSKLSVIRNLNDQVLFIDQGNRPLFEDMTDSDVRDNAPRTIFIISMYdDSQPRG |
| 1112 | | | rAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 286) |
| FUSE- | | | SEQ ID NO: 99 |
| 1113 | | | |
| FUSE- | | | YFGKLESKLSVIRNLNDQVLFIDQGNRPLFEDMTDSDVRDNAPRTIFIISKYGDSQPRG |
| 1116 | | | LAVTISVKVEKISTLSVENKIISFKEMNPPDNIKDTKSDIIFFQRSVPGHRNKVQFESS |
| | | | SYEGYFLAVEKERDLFKLILKKEDELGDRSIMFTVQNED (SEQ ID NO: 287) |
| FUSE- | | | SEQ ID NO: 99 |
| 1176 | | | |
| FUSE- | | | YFGKLqSKLSVIRNLNDQVLFIDQGNRPLFEDMTDSDVRDNAPRTIFIISMYdDSQPRG |
| 1177 | | | kAVTISVKVEKISTLSVENKIISFKEMNPPDNIKDTKSDIIFFQRSVPGHDNKMQFESS |
| | | | SYEGYFLAVEKERDLFKLILKKEDELGDRSIMFTVQNED (SEQ ID NO: 283) |

TABLE 2
Nucleotide sequences of respective polypeptides of Table IA.

| | Polynucleotide 1 | Polynucleotide 2 | Polynucleotide 3 |
|---|---|---|---|
| FUSE-422 | GAGCCCAAATCTTCTGACAAAACTCACACATGC | GAGCCCAAATCTTCTGACAAAACTCACACATGCCCACCGTGCCCAGCACCTGAACTCCTGGG | |
| | CCACCGTGCCCAGCACCTGAACTCCTGGGGGGA | GGGACCGTCAGTCTTCCTCTTCCCCCCAAAACCCAAGGACACCCTCATGATCTCCCGGACCC | |
| | CCGTCAGTCTTCCTCTTCCCCCCAAAACCCAAG | CCGAGGTCACATGCGTGGTGGTGGACGTGAGCCACGAAGACCCTGAGGTCAAGTTCAACTGG | |
| | GACACCCTCATGATCTCCCGGACCCCCGAGGTC | TACGTGGACGGCGTGGAGGTGCATAATGCCAAGACAAAGCCGCGGGAGGAGCAGTACAACAG | |
| | ACATGCGTGGTGGTGGACGTGAGCCACGAAGAC | CACGTACCGTGTGGTCAGCGTCCTCACCGTCCTGCACCAGGACTGGCTGAATGGCAAGGAGT | |
| | CCTGAGGTCAAGTTCAACTGGTACGTGGACGGC | ACAAGTGCAAGGTCTCCAACAAAGCCCTCCCAGCCCCCATCGAGAAAACCATCTCCAAAGCC | |
| | GTGGAGGTGCATAATGCCAAGACAAAGCCGCGG | AAAGGGCAGCCCCGAGAACCACAGGTGTACACCCTGCCCCCATCCCGGGAGGAGATGACCAA | |
| | GAGGAGCAGTACAACAGCACGTACCGTGTGGTC | GAACCAGGTCAGCCTGTGGTGCCTGGTCAAAGGCTTCTATCCCAGCGACATCGCCGTGGAGT | |
| | AGCGTCCTCACCGTCCTGCACCAGGACTGGCTG | GGGAGAGCAATGGGCAGCCGGAGAACAACTACAAGACCACGCCTCCCGTGCTGGACTCCGAC | |
| | AATGGCAAGGAGTACAAGTGCAAGGTCTCCAAC | GGCTCCTTCTTCCTCTACAGCAAGCTCACCGTGGACAAGAGCAGGTGGCAGCAGGGGAACGT | |
| | AAAGCCCTCCCAGCCCCCATCGAGAAAACCATC | CTTCTCATGCTCCGTGATGCATGAGGCTCTGCACAACCACTACACGCAGAAGAGCCTCTCCC | |
| | TCCAAAGCCAAAGGGCAGCCCCGAGAACCACAG | TGTCTCCGGGTAAAGCTGCTGAACCCGTGGAAGATAATTCTATTAATTTTGTGGCCATGAAG | |
| | GTGTACACCCTGCCCCCATCCCGGGAGGAGATG | TTCATTGATAATACTCTGTATTTCATCGCCGAAGATGATGAGAACGACGACGACGATAAATA | |
| | ACCAAGAACCAGGTCAGCCTGAGCTGCGCCGTC | TTTCGGAAAACTGGAGTCCAAGCTGAGCGTGATCAGGAACCTGAATGATCAGGTGCTGTTCA | |
| | AAAGGCTTCTATCCCAGCGACATCGCCGTGGAG | TTGACCAGGGCAACAGGCCCCTGTTTGAGGATATGACCGACAGCGACAGCAGGGATAACGCT | |
| | TGGGAGAGCAATGGGCAGCCGGAGAACAACTAC | CCCCGGACAATCTTCATCATCTCCAAGTACGGCGATTCCCAGCCCAGGGGACTGGCCGTGAC | |
| | AAGACCACGCCTCCCGTGCTGGACTCCGACGGC | CATCAGCGTGAAATCTGAGAAGATCTCCACCCTGAGCTCCGAAAACAAGATCATCAGCTTCA | |
| | TCCTTCTTCCTCGTGAGCAAGCTCACCGTGGAC | AGGAGATGAACCCCCCCGACAACATTAAGGACACCAAATCCGACATCATCTTCTTCCAGAGA | |
| | AAGAGCAGGTGGCAGCAGGGGAACGTCTTCTCA | TCCGTGCCCGGCCACGACAACAAAGTGCAGTTCGAGTCCTCCTCCTACGAGGGCTACTTTCT | |
| | TGCTCCGTGATGCATGAGGCTCTGCACAACCGG | GGCCTCCGAAAAAGAAAGGGACCTGTTCAAGCTGATCCTGAAGAAGGAGGACGAACTGGGGG | |
| | TTCACGCAGAAGAGCCTCTCCCTGTCTCCGGGT | ACAGGAGCATCATGTTCACCGTGCAGAACGAGGACATCATGTTCACCGTGCAGAACGAGGAC | |
| | AAA (SEQ ID NO: 114) | (SEQ ID NO: 133) | |
| FUSE-423 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGCCATTAATTTTGTGGCCATGAAA | |
| | | TTCATCGATAACACCCTGTACTTTATTGCCGAGGATGATGAAAATGATGATGATGACAAATA | |
| | | CTTCGGCAAACTGGAATCCAAGCTGTCCGTGATCAGGAACCTGAATGATCAGGTGCTGTTCA | |
| | | TTGACCAGGGCAACCGCCCCCTGTTCGAGGACATGACCGATTCCGATGCCAGGGACAACGCA | |
| | | CCCCGGACCATCTTCATCATCTCTAAGTACGGAGACTCCCAGCCAAGGGGCCTGGCCGTGAC | |
| | | CATCTCCGTGAAGGCCGAGAAAATTTCCACCCTGTCCGCCGAAAACAAAATCATCTCCTTCA | |
| | | AAGAGATGAACCCCCCCGACAACATCAAGGACACCAAATCCGACATCATCTTCTTCCAGCGC | |
| | | TCCGTGCCCGGCCACGACAACAAAGTGCAGTTCGAGTCCAGCTCCTACGAGGGCTACTTCCT | |
| | | GGCCGCCGAAAAGGAAAGGGACCTGTTCAAGCTGATCCTGAAGAAGGAGGACGAGCTGGGTG | |
| | | ACAGGAGCATCATGTTCACCGTGCAGAACGAGGAC (SEQ ID NO: 134) | |
| FUSE-424 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTTGTGGCCATGAAA | |
| | | TTTATTGACAACACCCTGTATTTCATCGCCGAAGATGATGAAAACGATGATGATGATAAGTA | |
| | | CTTTGGAAAACTGGAATCAAAGCTGAGCGTGATCCGGAACCTGAATGATCAGGTGCTGTTCA | |
| | | TCGACCAGGGCAACAGGCCCCTGTTTGAGGACATGACCGACTCCGACGTGAGGGACAACGCT | |
| | | CCCCGGACCATTTTCATCATCTCTAAGTACGGCGACAGCCAGCCCAGGGGACTGGCCGTGAC | |
| | | CATTTCCGTGAAGGTGGAGAAGATTTCCACACTGTCCGTGGAGAACAAAATCATCTCCTTCA | |
| | | AAGAGATGAACCCCCCCGACAACATCAAAGACACAAAAAGCGATATTATCTTCTTCCAGCGC | |
| | | TCTGTGCCCGGCCACGACAACAAGGTGCAGTTCGAGAGCTCCAGCTACGAGGGCTACTTTCT | |
| | | GGCCGTGGAGAAGGAAAGGGACCTGTTCAAACTGATTCTGAAGAAGGAGGACGAACTGGGGG | |
| | | ACAGGTCCATCATGTTCACCGTGCAGAATGAAGAC (SEQ ID NO: 135) | |
| FUSE-441 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGGAGATGACGACGACAAGTACTTTGGAAAGCTGGAGAGCAAGCTGAGT | |
| | | GTGATCAGGAACCTGAACGACCAGGTGCTGTTTATCGACCAGGGCAATAGGCCACTGTTTGA | |
| | | GGATATGACAGATTCCGACGTGAGGGACAACGCTCCCCGGACCATCTTCATTATCAGTAAGT | |
| | | ATGGCGACTCCCAGCCAAGGGGCCTGGCTGTGACCATCAGCGTGAAAGTGGAGAAGATTAGC | |
| | | ACACTGTCCGTGGAGAACAAGATCATCTCCTTCAAGGAGATGAACCCCCCCGACAACATCAA | |
| | | AGACACCAAGTCCGACATCATTTTCTTCCAGAGGTCCGTGCCTGGACACGACAACAAAGTGC | |
| | | AGTTCGAGTCCTCCTCCTACGAGGGCTACTTCCTGGCCGTGGAAAAAGAGCGCGACCTGTTC | |
| | | AAGCTGATCCTGAAGAAGGAGGACGAGCTGGGGGACAGGAGCATCATGTTCACCGTGCAGAA | |
| | | CGAGGAC (SEQ ID NO: 136) | |
| FUSE-442 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTTGTGGCCATGAAA | |
| | | TTTATTGACAACACCCTGTATTTCATCGCCGAAGATGATGAAAACGATGATGATGATAAGTA | |
| | | CTTTGGAAAACTGGAATCAAAGCTGAGCGTGATCCGGAACCTGAATGATCAGGTGCTGTTCA | |
| | | TCGACCAGGGCAACAGGCCCCTGTTTGAGGACATGACCGACTCCGACGTGAGGGACAACGCT | |
| | | CCCCGGACCATTTTCATCATCTCTATGTACAAGGACAGCCAGCCCAGGGGTATGGCCGTGAC | |
| | | CATTAGCGTGAAGGTGGAGAAGATTAGCACACTGTCCGTGGAGAACAAAATCATCTCCTTCA | |
| | | AGGAAATGAACCCCCCCGACAACATCAAAGACACCAAGAGCGACATCATCTTCTTCCAGAGG | |
| | | AGCGTGCCCGGGCACGACAACAAAATGCAGTTCGAATCCAGCTCCTACGAGGGCTACTTCCT | |
| | | GGCCGTGGAGAAAGAGAGGGACCTGTTCAAGCTGATCCTGAAGAAGGAGGACGAGCTGGGGG | |
| | | ACAGGAGCATCATGTTCACCGTGCAGAACGAGGAC (SEQ ID NO: 137) | |
| FUSE-462 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATGTGATCAATTTCGTGGCCATGAAG | |
| | | TTCATTGACAACACCCTGTACTTCATCGCTGAAGATGATGAAAACATCGAGCAGGACTATTT | |
| | | CGGAAAACTGGAAAGCAAACTGAGCGTGATCAGGAACCTGAACGACCAGGTGCTGTTTATCG | |
| | | ACCAGGGCAACAGGCCCCTGTTCGAAGATATGACCGACAGCGACGTCAGGGACAACGCTCCT | |
| | | AGAACAATCTTCATCATCTCCAAGTACGGCGACTCCCAGCCAAGGGGACTGGCTGTGACCAT | |
| | | TTCCGTGAAAGTGGAGAAGATCTCCACACTGAGCGTGGAAAACAAAATCATCTCCTTCAAGG | |
| | | AGATGAACCCCCCCGACAACATTAAAGACACCAAAAGCGATATCATCTTCTTCCAGAGGAGT | |
| | | GTGCCCGGCCACGATAACAAAGTGCAGTTCGAGAGCTCCTCCTACGAGGGATACTTTCTGGC | |
| | | CGTCGAAAAGGAGAGGGACCTGTTCAAGCTGATCCTGAAGAAGGAGGACGAGCTGGGGGACA | |
| | | GATCAATTATGTTCACCGTGCAGAATGAGGAC (SEQ ID NO: 138) | |
| FUSE-480 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATTCTATTAACTTCGTGGCCATGAAG | |
| | | TTCATCGATAATACTCTGTACTTCATCGCCGAAGATGATGAAAATGACGACGATGACAAGTA | |
| | | CTTCGGTAAACTGGAGTCCAAGCTGTCCGTGATCAGGAACCTGAATGATCAGGTGCTGTTCA | |
| | | TTGACCAGGGCAACAGGCCCCTGTTTGAGGATATGACCGACTCCGACTCTAGAGATAACGCC | |
| | | CCCCGAACAATCTTCATCATCAGTATGTACAAGGACTCCCAGCCCAGGGGAATGGCTGTGAC | |
| | | CATTAGCGTGAAGTCCGAGAAGATTAGCACCCTGTCCTCCGAGAACAAGATCATCTCCTTCA | |
| | | AAGAGATGAACCCCCCCGACAACATCAAAGACACCAAGAGCGACATCATCTTCTTCCAGAGG | |
| | | AGCGTGCCCGGCCATGATAACAAAATGCAGTTCGAAAGCTCCTCCTACGAGGGCTACTTCCT | |
| | | GGCATCCGAAAAGGAAAGGGACCTGTTCAAACTGATCCTGAAGAAGGAGGATGAGCTGGGGG | |
| | | ACAGGTCCATCATGTTCACCGTCCAGAACGAGGAC (SEQ ID NO: 139) | |
| FUSE-481 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATGCCATTAATTTCGTGGCCATGAAG | |
| | | TTCATTGACAACACCCTGTACTTCATCGCCGAGGATGATGAGAACGACGACGACGATAAATA | |
| | | TTTCGGAAAGCTGGAGTCCAAGCTGAGCGTGATCAGGAACCTGAATGATCAGGTGCTGTTCA | |
| | | TTGACCAGGGCAACAGGCCCCTGTTTGAGGATATGACCGACAGCGACGCCAGGGATAACGCT | |
| | | CCTAGAACAATCTTCATCATCTCTATGTACAAGGACTCCCAGCCCAGGGGAATGGCTGTGAC | |
| | | CATTTCCGTGAAGGCCGAGAAGATTTCCACCCTGTCAGCCGAAAACAAGATCATCTCCTTCA | |
| | | AAGAGATGAACCCCCCCGACAACATCAAAGACACCAAATCCGACATCATCTTCTTCCAGCGC | |
| | | AGCGTGCCCGGACATGATAACAAAATGCAGTTCGAAAGCTCCTCCTACGAGGGCTACTTCCT | |
| | | CGCAGCAGAAAAAGAAAGGGACCTGTTCAAGCTGATCCTGAAGAAGGAGGACGAGCTGGGGG | |
| | | ACAGAAGCATCATGTTCACCGTGCAGAACGAAGAC (SEQ ID NO: 140) | |
| FUSE-484 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATGTGATCAATTTCGTGGCCATGAAG | |
| | | TTCATTGACAACACCCTGTACTTCATCGCTGAAGATGATGAAAACGACGATGATGATAAGTA | |
| | | CTTCGGAAAGCTGGAGTCCAAGCTGAGCGTGATTAGGAACCTGAATGACCAGGTGCTGTTCA | |
| | | TTGACCAGGGCAACAGGCCCCTCTTTGAGGATATGACCGACTCCGATGTGAGGGACAACGCC | |
| | | CCTAGAACAATCTTCATCATCTCCAAGTACGGCGACTCCGGAGCAAGGGGACTGGCTGTGAC | |
| | | CATTTCCGTGAAAGTGGAGAAGATTTCCACCCTGTCCGTGGAGAACAAGATCATTTCCTTCA | |
| | | AAGAGATGAACCCCCCAGACAACATTAAGGACACAAAGAGCGATATCATCTTCTTCGAGCGC | |
| | | GATGTGCCCGGGCATTCTGGAAAAGTGCAGTTCGAGTCCTCCTCCTACGAGGGTTACTTTCT | |
| | | GGCCGTCGAAAAGGAAAGGGACCTGTTCAAGCTGATCCTGAAGAAGGAGGACGAGCTGGGGG | |
| | | ACAGATCCATCATGTTCACCGTGCAGAATGAGGAC (SEQ ID NO: 141) | |
| FUSE-485 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTCGTGGCCATGAAG | |
| | | TTCATTGACAACACCCTGTACTTCATCGCCGAAGATGATGAAAACATCGAACAGGACTACTT | |
| | | CGGAAAACTGGAATCCAAACTGAGCGTGATCAGGAACCTGAACGACCAGGTGCTGTTCATCG | |
| | | ACCAGGGCAATCGCCCCCTCTTTGAGGACATGACCGACTCCGACGTCCGGGACAACGCTCCC | |
| | | AGGACCATCTTCATCATCTCCATGTACAAAGACTCCCAGCCAAGGGGCATGGCCGTGACCAT | |
| | | CTCCGTCAAGGTGGAGAAGATCTCCACCCTGTCCGTGGAAAACAAGATCATCTCCTTCAAAG | |
| | | AGATGAACCCCCCCGACAACATCAAAGACACAAAATCCGACATCATCTTCTTCCAGCGCTCC | |
| | | GTGCCTGGGCACGACAACAAAATGCAGTTCGAATCCTCCTCCTACGAGGGCTACTTCCTCGC | |
| | | CGTCGAAAAAGAAAGAGACTTGTTCAAGCTCATCCTCAAGAAGGAGGACGAGCTCGGGGACA | |
| | | GGTCAATCATGTTCACCGTGCAGAACGAGGAC (SEQ ID NO: 142) | |
| FUSE-486 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTCGTGGCCATGAAG | |
| | | TTCATTGACAACACCCTGTACTTCATCGCCGAAGATGATGAAAACGGACCCCTGGGAGTGAG | |
| | | GGGAGGATACTTCGGAAAGCTGGAGAGCAAGCTGAGCGTGATCAGGAATCTGAACGACCAGG | |
| | | TGCTGTTCATCGACCAGGGCAACAGGCCCCTGTTCGAGGACATGACAGACAGTGACGTGAGG | |
| | | GACAACGCCCCCCGGACCATCTTCATCATCAGTATGTACAAGGACTCCCAGCCAAGGGGCAT | |
| | | GGCCGTGACCATCTCCGTGAAAGTGGAGAAAATCAGCACACTGAGCGTGGAGAACAAGATCA | |
| | | TCTCCTTCAAGGAGATGAACCCCCCCGACAACATCAAAGACACAAAATCCGACATCATCTTC | |
| | | TTCCAGAGGAGCGTGCCCGGCCACGACAACAAAATGCAGTTCGAATCCAGCTCCTACGAGGG | |
| | | CTACTTCCTCGCCGTCGAAAAGGAAAGGGACCTGTTCAAGCTCATCCTGAAGAAGGAGGACG | |
| | | AGCTGGGGGACAGGAGCATCATGTTCACCGTCCAGAACGAGGAC (SEQ ID NO: 143) | |
| FUSE-487 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTCGTGGCCATGAAG | |
| | | TTCATTGACAACACCCTGTACTTCATCGCCGAAGATGATGAAAACGTGCATATGCCTCTGGG | |
| | | CTTTCTGGGACCCGGAGGCTATTTTGGAAAGCTGGAGAGCAAGCTGTCCGTGATCCGCAACC | |
| | | TGAACGACCAGGTGCTGTTCATCGACCAGGGAAACAGGCCCCTGTTCGAGGACATGACCGAC | |
| | | TCTGACGTGAGGGACAACGCCCCCAGGACAATCTTCATCATCTCCATGTACAAGGACTCCCA | |
| | | GCCAAGGGGCATGGCCGTGACCATCTCCGTGAAGGTGGAGAAGATCTCCACCCTGAGCGTGG | |
| | | AGAACAAGATCATCAGCTTCAAGGAGATGAACCCCCCCGACAACATCAAAGACACAAAATCC | |
| | | GACATCATCTTCTTCCAGAGGAGCGTGCCTGGCCACGACAACAAAATGCAGTTTGAGTCCTC | |
| | | CTCCTACGAGGGCTACTTCCTGGCCGTCGAAAAGGAAAGGGACCTGTTCAAGCTGATCCTGA | |
| | | AGAAGGAGGACGAGCTGGGCGACAGGTCAATTATGTTCACCGTGCAGAACGAGGAC (SEQ | |
| | | ID NO: 144) | |
| FUSE-499 | (SEQ ID NO: 114) | GCTGCTGAACCTGTGGAAGATAATGTGATCAATTTCGTGGCCATGAAGTTCATTGACAACAC | |
| | | CCTGTACTTCATCGCTGAAGATGATGAAAACCTGGAGTCCGACTACTTCGGAAAACTGGAAT | |
| | | CCAAACTGAGCGTGATCCGGAACCTGAACGATCAGGTGCTGTTCATCGACCAGGGCAATCGG | |
| | | CCTCTGTTTGAGGACATGACCGACAGCGACGTGAGGGATAACGCTCCTAGGACCATCTTCAT | |
| | | CATCTCCATGTATAAGGACTCCCAGCCAAGGGGCATGGCCGTGACCATCTCCGTGAAAGTGG | |
| | | AGAAGATCTCCACACTGTCCGTGGAAAACAAGATCATCTCCTTCAAAGAGATGAACCCCCCC | |
| | | GACAACATCAAAGACACAAAAAGCGATATTATCTTCTTCCAGCGGAGCGTGCCCGGGCACGA | |
| | | CAACAAGATGCAGTTCGAAAGCTCCTCCTACGAGGGATACTTTCTGGCCGTCGAAAAGGAAA | |
| | | GGGACCTGTTCAAACTGATTCTGAAGAAGGAGGACGAGCTGGGGGACAGAAGCATCATGTTC | |
| | | ACCGTGCAGAACGAGGACGAGCCCAAATCTTCTGACAAAACTCACACATGCCCACCGTGCCC | |
| | | AGCACCTGAACTCCTGGGGGGACCGTCAGTCTTCCTCTTCCCCCCAAAACCCAAGGACACCC | |
| | | TCATGATCTCCCGGACCCCCGAGGTCACATGCGTGGTGGTGGACGTGAGCCACGAAGACCCT | |
| | | GAGGTCAAGTTCAACTGGTACGTGGACGGCGTGGAGGTGCATAATGCCAAGACAAAGCCGCG | |
| | | GGAGGAGCAGTACAACAGCACGTACCGTGTGGTCAGCGTCCTCACCGTCCTGCACCAGGACT | |
| | | GGCTGAATGGCAAGGAGTACAAGTGCAAGGTCTCCAACAAAGCCCTCCCAGCCCCCATCGAG | |
| | | AAAACCATCTCCAAAGCCAAAGGGCAGCCCCGAGAACCACAGGTGTACACCCTGCCCCCATC | |
| | | CCGGGAGGAGATGACCAAGAACCAGGTCAGCCTGTGGTGCCTGGTCAAAGGCTTCTATCCCA | |
| | | GCGACATCGCCGTGGAGTGGGAGAGCAATGGGCAGCCGGAGAACAACTACAAGACCACGCCT | |
| | | CCCGTGCTGGACTCCGACGGCTCCTTCTTCCTCTACAGCAAGCTCACCGTGGACAAGAGCAG | |
| | | GTGGCAGCAGGGGAACGTCTTCTCATGCTCCGTGATGCATGAGGCTCTGCACAACCACTACA | |
| | | CGCAGAAGAGCCTCTCCCTGTCTCCGGGTAAA (SEQ ID NO: 145) | |
| FUSE-500 | (SEQ ID NO: 114) | TACTTCGGCAAGCTGGAGTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGATAACG | |
| | | CTCCAAGGACAATCTTCATCATCTCCATGTACAAGGACTCCCAGCCCAGGGGAATGGCTGTG | |
| | | ACCATTTCCGTGAAAGTGGAGAAGATTAGCACCCTGAGCGTGGAGAACAAGATCATCTCCTT | |
| | | CAAGGAAATGAACCCCCCCGACAACATCAAAGACACCAAATCCGACATCATCTTCTTCCAGA | |
| | | GGTCCGTGCCTGGCCACGATAACAAAATGCAGTTCGAGTCCTCCTCCTACGAGGGCTACTTC | |
| | | CTGGCAGTCGAAAAGGAGAGGGACCTGTTCAAGCTCATCCTGAAGAAGGAGGACGAGCTGGG | |
| | | GGACAGATCCATCATGTTCACCGTGCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 146) | |
| FUSE-501 | GACGCTCACAAATCCGAAGTGGCACACAGGTTT | | |
| | AAGGACCTGGGAGAAGAGAACTTCAAAGCCCTG | | |
| | GTGCTGATTGCATTTGCCCAGTATCTGCAGCAG | | |
| | TGTCCCTTTGAGGACCACGTGAAGCTGGTGAAC | | |
| | GAGGTGACCGAATTTGCCAAGACTTGCGTGGCC | | |
| | GACGAGTCCGCAGAGAACTGCGACAAGTCCCTG | | |
| | CATACCCTGTTTGGCGACAAGCTGTGTACAGTG | | |
| | GCCACCCTGCGGGAGACATACGGTGAGATGGCC | | |
| | GACTGCTGCGCCAAGCAGGAACCCGAGAGAAAC | | |
| | GAATGCTTCCTGCAGCACAAGGACGACAACCCC | | |
| | AACCTGCCCCGGCTGGTGAGGCCCGAAGTGGAC | | |
| | GTGATGTGTACCGCCTTTCACGACAACGAAGAG | | |
| | ACATTTCTGAAGAAGTACCTGTACGAGATCGCC | | |
| | AGAAGGCACCCTTACTTTTACGCCCCCGAGCTG | | |
| | CTGTTCTTTGCCAAGAGGTATAAGGCCGCCTTT | | |
| | ACTGAGTGCTGCCAGGCAGCCGACAAAGCCGCC | | |
| | TGCCTGCTGCCCAAGCTGGACGAGCTGCGCGAC | | |
| | GAGGGCAAGGCCTCCTCCGCTAAGCAGAGGCTG | | |
| | AAGTGCGCATCCCTGCAGAAATTTGGCGAGAGA | | |
| | GCCTTCAAGGCATGGGCCGTGGCTAGGCTGTCC | | |
| | CAGAGGTTTCCAAAGGCCGAATTTGCCGAGGTG | | |
| | TCCAAGCTGGTGACAGACCTGACCAAGGTGCAC | | |
| | ACCGAGTGTTGTCACGGAGACTTGCTGGAGTGC | | |
| | GCCGACGACAGGGCCGACCTGGCTAAGTACATC | | |
| | TGTGAGAACCAGGACTCTATCTCCTCCAAGCTG | | |
| | AAGGAGTGCTGCGAGAAGCCCCTGCTGGAGAAA | | |
| | AGCCACTGTATTGCTGAGGTGGAGAACGACGAG | | |
| | ATGCCCGCCGACCTGCCCTCTCTGGCCGCTGAT | | |
| | TTCGTGGAGTCCAAGGACGTGTGTAAGAACTAC | | |
| | GCCGAGGCCAAAGACGTGTTCCTGGGGATGTTT | | |
| | CTGTATGAGTACGCTAGGAGGCACCCCGATTAC | | |
| | TCCGTGGTGCTGCTGCTGAGGCTGGCAAAAACA | | |
| | TACGAAACCACCCTGGAGAAGTGCTGTGCCGCT | | |
| | GCTGACCCCCACGAGTGCTATGCCAAGGTGTTC | | |
| | GACGAGTTCAAACCCCTGGTGGAGGAACCCCAG | | |
| | AACCTGATCAAGCAGAACTGCGAGCTGTTCGAA | | |
| | CAGCTGGGCGAGTACAAATTCCAGAACGCACTG | | |
| | CTGGTGAGGTACACCAAGAAAGTGCCCCAGGTG | | |
| | AGCACCCCCACCCTGGTGGAGGTGTCCAGGAAC | | |
| | CTGGGCAAAGTGGGCAGCAAATGCTGCAAGCAC | | |
| | CCCGAGGCCAAAAGAATGCCCTGTGCAGAGGAC | | |
| | TACCTGAGTGTGGTGCTGAACCAGCTGTGCGTG | | |
| | CTGCACGAGAAGACACCCGTGAGCGACAGGGTG | | |
| | ACCAAGTGTTGTACCGAGTCCCTGGTGAACAGA | | |
| | AGGCCCTGTTTTAGTGCCCTGGAAGTGGATGAG | | |
| | ACTTACGTGCCTAAGGAGTTCAACGCAGAGACA | | |
| | TTCACCTTCCATGCCGATATTTGCACCCTGTCC | | |
| | GAAAAGGAAAGACAGATCAAGAAACAGACCGCC | | |
| | CTGGTGGAGCTGGTGAAACACAAGCCCAAGGCC | | |
| | ACCAAGGAACAGCTGAAAGCCGTGATGGACGAC | | |
| | TTCGCCGCCTTCGTGGAGAAATGCTGCAAGGCC | | |
| | GACGACAAAGAAACCTGCTTCGCAGAGGAAGGA | | |
| | AAGAAACTGGTGGCAGCCAGCCAGGCCGCCCTG | | |
| | GGACTGGCTGCTGAACCTGTGGAAGATAATGTG | | |
| | ATCAATTTCGTGGCCATGAAGTTCATTGACAAC | | |
| | ACCCTGTACTTCATCGCTGAAGATGATGAAAAC | | |
| | GACGATGATGATAAGTACTTCGGAAAGCTGGAG | | |
| | TCCAAGCTGAGCGTGATTAGGAACCTGAATGAC | | |
| | CAGGTGCTGTTCATTGACCAGGGCAACAGGCCC | | |
| | CTCTTTGAGGATATGACCGACTCCGATGTGAGG | | |
| | GACAACGCCCCTAGAACAATCTTCATCATCTCC | | |
| | ATGTACAAGGACTCCCAGCCAAGGGGAATGGCC | | |
| | GTGACCATTTCCGTGAAAGTCGAGAAAATTTCC | | |
| | ACCCTGTCCGTGGAGAACAAGATCATCTCCTTC | | |
| | AAAGAAATGAACCCCCCCGACAACATCAAGGAC | | |
| | ACCAAATCCGACATCATCTTCTTCCAGCGCTCC | | |
| | GTGCCAGGCCATGATAACAAAATGCAGTTCGAG | | |
| | AGCAGCTCCTACGAGGGCTACTTCCTGGCAGTC | | |
| | GAAAAGGAAAGGGACCTGTTCAAGCTGATCCTG | | |
| | AAGAAGGAGGACGAGCTGGGGGACAGAAGCATC | | |
| | ATGTTCACCGTGCAGAACGAAGAC (SEQ ID | | |
| | NO: 115) | | |
| FUSE-502 | GACGCTCACAAATCCGAAGTGGCACACAGGTTT | | |
| | AAGGACCTGGGAGAAGAGAACTTCAAAGCCCTG | | |
| | GTGCTGATTGCATTTGCCCAGTATCTGCAGCAG | | |
| | TGTCCCTTTGAGGACCACGTGAAGCTGGTGAAC | | |
| | GAGGTGACCGAATTTGCCAAGACTTGCGTGGCC | | |
| | GACGAGTCCGCAGAGAACTGCGACAAGTCCCTG | | |
| | CATACCCTGTTTGGCGACAAGCTGTGTACAGTG | | |
| | GCCACCCTGCGGGAGACATACGGTGAGATGGCC | | |
| | GACTGCTGCGCCAAGCAGGAACCCGAGAGAAAC | | |
| | GAATGCTTCCTGCAGCACAAGGACGACAACCCC | | |
| | AACCTGCCCCGGCTGGTGAGGCCCGAAGTGGAC | | |
| | GTGATGTGTACCGCCTTTCACGACAACGAAGAG | | |
| | ACATTTCTGAAGAAGTACCTGTACGAGATCGCC | | |
| | AGAAGGCACCCTTACTTTTACGCCCCCGAGCTG | | |
| | CTGTTCTTTGCCAAGAGGTATAAGGCCGCCTTT | | |
| | ACTGAGTGCTGCCAGGCAGCCGACAAAGCCGCC | | |
| | TGCCTGCTGCCCAAGCTGGACGAGCTGCGCGAC | | |
| | GAGGGCAAGGCCTCCTCCGCTAAGCAGAGGCTG | | |
| | AAGTGCGCATCCCTGCAGAAATTTGGCGAGAGA | | |
| | GCCTTCAAGGCATGGGCCGTGGCTAGGCTGTCC | | |
| | CAGAGGTTTCCAAAGGCCGAATTTGCCGAGGTG | | |
| | TCCAAGCTGGTGACAGACCTGACCAAGGTGCAC | | |
| | ACCGAGTGTTGTCACGGAGACTTGCTGGAGTGC | | |
| | GCCGACGACAGGGCCGACCTGGCTAAGTACATC | | |
| | TGTGAGAACCAGGACTCTATCTCCTCCAAGCTG | | |
| | AAGGAGTGCTGCGAGAAGCCCCTGCTGGAGAAA | | |
| | AGCCACTGTATTGCTGAGGTGGAGAACGACGAG | | |
| | ATGCCCGCCGACCTGCCCTCTCTGGCCGCTGAT | | |
| | TTCGTGGAGTCCAAGGACGTGTGTAAGAACTAC | | |
| | GCCGAGGCCAAAGACGTGTTCCTGGGGATGTTT | | |
| | CTGTATGAGTACGCTAGGAGGCACCCCGATTAC | | |
| | TCCGTGGTGCTGCTGCTGAGGCTGGCAAAAACA | | |
| | TACGAAACCACCCTGGAGAAGTGCTGTGCCGCT | | |
| | GCTGACCCCCACGAGTGCTATGCCAAGGTGTTC | | |
| | GACGAGTTCAAACCCCTGGTGGAGGAACCCCAG | | |
| | AACCTGATCAAGCAGAACTGCGAGCTGTTCGAA | | |
| | CAGCTGGGCGAGTACAAATTCCAGAACGCACTG | | |
| | CTGGTGAGGTACACCAAGAAAGTGCCCCAGGTG | | |
| | AGCACCCCCACCCTGGTGGAGGTGTCCAGGAAC | | |
| | CTGGGCAAAGTGGGCAGCAAATGCTGCAAGCAC | | |
| | CCCGAGGCCAAAAGAATGCCCTGTGCAGAGGAC | | |
| | TACCTGAGTGTGGTGCTGAACCAGCTGTGCGTG | | |
| | CTGCACGAGAAGACACCCGTGAGCGACAGGGTG | | |
| | ACCAAGTGTTGTACCGAGTCCCTGGTGAACAGA | | |
| | AGGCCCTGTTTTAGTGCCCTGGAAGTGGATGAG | | |
| | ACTTACGTGCCTAAGGAGTTCAACGCAGAGACA | | |
| | TTCACCTTCCATGCCGATATTTGCACCCTGTCC | | |
| | GAAAAGGAAAGACAGATCAAGAAACAGACCGCC | | |
| | CTGGTGGAGCTGGTGAAACACAAGCCCAAGGCC | | |
| | ACCAAGGAACAGCTGAAAGCCGTGATGGACGAC | | |
| | TTCGCCGCCTTCGTGGAGAAATGCTGCAAGGCC | | |
| | GACGACAAAGAAACCTGCTTCGCAGAGGAAGGA | | |
| | AAGAAACTGGTGGCAGCCAGCCAGGCCGCCCTG | | |
| | GGACTGGCTGCTGAACCTGTGGAAGATAATGTG | | |
| | ATCAATTTCGTGGCCATGAAGTTCATTGACAAC | | |
| | ACCCTGTACTTCATCGCTGAAGATGATGAAAAC | | |
| | GACGATGATGATAAGTACTTCGGAAAGCTGGAG | | |
| | TCCAAGCTGAGCGTGATTAGGAACCTGAATGAC | | |
| | CAGGTGCTGTTCATTGACCAGGGCAACAGGCCC | | |
| | CTCTTTGAGGATATGACCGACTCCGATGTGAGG | | |
| | GACAACGCCCCTAGAACAATCTTCATCATCTCC | | |
| | AAGTACGGCGACTCCCAGCCAAGAGGACTGGCT | | |
| | GTGACAATTAGCGTGAAAGTCGAGAAGATTTCC | | |
| | ACCCTGTCCGTGGAGAACAAAATCATCTCCTTC | | |
| | AAGGAGATGAACCCCCCCGACAACATCAAGGAC | | |
| | ACCAAAAGCGATATCATCTTCTTCCAGAGGTCC | | |
| | GTCCCCGGTCACGATAACAAAGTGCAGTTCGAG | | |
| | TCCTCCTCCTACGAGGGTTACTTTCTGGCCGTC | | |
| | GAAAAGGAGCGGGACCTGTTTAAGCTGATCCTG | | |
| | AAGAAGGAGGACGAGCTGGGTGACAGGTCCATC | | |
| | ATGTTCACCGTCCAGAATGAGGAC (SEQ ID | | |
| | NO: 116) | | |
| FUSE-503 | GACGCTCACAAATCCGAAGTGGCACACAGGTTT | | |
| | AAGGACCTGGGAGAAGAGAACTTCAAAGCCCTG | | |
| | GTGCTGATTGCATTTGCCCAGTATCTGCAGCAG | | |
| | TGTCCCTTTGAGGACCACGTGAAGCTGGTGAAC | | |
| | GAGGTGACCGAATTTGCCAAGACTTGCGTGGCC | | |
| | GACGAGTCCGCAGAGAACTGCGACAAGTCCCTG | | |
| | CATACCCTGTTTGGCGACAAGCTGTGTACAGTG | | |
| | GCCACCCTGCGGGAGACATACGGTGAGATGGCC | | |
| | GACTGCTGCGCCAAGCAGGAACCCGAGAGAAAC | | |
| | GAATGCTTCCTGCAGCACAAGGACGACAACCCC | | |
| | AACCTGCCCCGGCTGGTGAGGCCCGAAGTGGAC | | |
| | GTGATGTGTACCGCCTTTCACGACAACGAAGAG | | |
| | ACATTTCTGAAGAAGTACCTGTACGAGATCGCC | | |
| | AGAAGGCACCCTTACTTTTACGCCCCCGAGCTG | | |
| | CTGTTCTTTGCCAAGAGGTATAAGGCCGCCTTT | | |
| | ACTGAGTGCTGCCAGGCAGCCGACAAAGCCGCC | | |
| | TGCCTGCTGCCCAAGCTGGACGAGCTGCGCGAC | | |
| | GAGGGCAAGGCCTCCTCCGCTAAGCAGAGGCTG | | |
| | AAGTGCGCATCCCTGCAGAAATTTGGCGAGAGA | | |
| | GCCTTCAAGGCATGGGCCGTGGCTAGGCTGTCC | | |
| | CAGAGGTTTCCAAAGGCCGAATTTGCCGAGGTG | | |
| | TCCAAGCTGGTGACAGACCTGACCAAGGTGCAC | | |
| | ACCGAGTGTTGTCACGGAGACTTGCTGGAGTGC | | |
| | GCCGACGACAGGGCCGACCTGGCTAAGTACATC | | |
| | TGTGAGAACCAGGACTCTATCTCCTCCAAGCTG | | |
| | AAGGAGTGCTGCGAGAAGCCCCTGCTGGAGAAA | | |
| | AGCCACTGTATTGCTGAGGTGGAGAACGACGAG | | |
| | ATGCCCGCCGACCTGCCCTCTCTGGCCGCTGAT | | |
| | TTCGTGGAGTCCAAGGACGTGTGTAAGAACTAC | | |
| | GCCGAGGCCAAAGACGTGTTCCTGGGGATGTTT | | |
| | CTGTATGAGTACGCTAGGAGGCACCCCGATTAC | | |
| | TCCGTGGTGCTGCTGCTGAGGCTGGCAAAAACA | | |
| | TACGAAACCACCCTGGAGAAGTGCTGTGCCGCT | | |
| | GCTGACCCCCACGAGTGCTATGCCAAGGTGTTC | | |
| | GACGAGTTCAAACCCCTGGTGGAGGAACCCCAG | | |
| | AACCTGATCAAGCAGAACTGCGAGCTGTTCGAA | | |
| | CAGCTGGGCGAGTACAAATTCCAGAACGCACTG | | |
| | CTGGTGAGGTACACCAAGAAAGTGCCCCAGGTG | | |
| | AGCACCCCCACCCTGGTGGAGGTGTCCAGGAAC | | |
| | CTGGGCAAAGTGGGCAGCAAATGCTGCAAGCAC | | |
| | CCCGAGGCCAAAAGAATGCCCTGTGCAGAGGAC | | |
| | TACCTGAGTGTGGTGCTGAACCAGCTGTGCGTG | | |
| | CTGCACGAGAAGACACCCGTGAGCGACAGGGTG | | |
| | ACCAAGTGTTGTACCGAGTCCCTGGTGAACAGA | | |
| | AGGCCCTGTTTTAGTGCCCTGGAAGTGGATGAG | | |
| | ACTTACGTGCCTAAGGAGTTCAACGCAGAGACA | | |
| | TTCACCTTCCATGCCGATATTTGCACCCTGTCC | | |
| | GAAAAGGAAAGACAGATCAAGAAACAGACCGCC | | |
| | CTGGTGGAGCTGGTGAAACACAAGCCCAAGGCC | | |
| | ACCAAGGAACAGCTGAAAGCCGTGATGGACGAC | | |
| | TTCGCCGCCTTCGTGGAGAAATGCTGCAAGGCC | | |
| | GACGACAAAGAAACCTGCTTCGCAGAGGAAGGA | | |
| | AAGAAACTGGTGGCAGCCAGCCAGGCCGCCCTG | | |
| | GGACTGGGAGATGACGACGACAAGTACTTTGGA | | |
| | AAGCTGGAGAGCAAGCTGAGTGTGATCAGGAAC | | |
| | CTGAACGACCAGGTGCTGTTTATCGACCAGGGC | | |
| | AATAGGCCACTGTTTGAGGATATGACAGATTCC | | |
| | GACGTGAGGGACAACGCTCCCAGAACAATTTTC | | |
| | ATCATCTCTATGTACAAGGACTCCCAGCCAAGG | | |
| | GGCATGGCTGTGACCATTTCCGTGAAGGTGGAG | | |
| | AAGATTTCCACCCTGTCTGTGGAGAACAAGATC | | |
| | ATCTCCTTCAAAGAGATGAACCCCCCCGACAAC | | |
| | ATCAAAGACACCAAAAGCGATATCATCTTCTTC | | |
| | CAGAGGTCCGTGCCCGGTCACGATAACAAAATG | | |
| | CAGTTCGAAAGCTCCTCCTACGAGGGATACTTT | | |
| | CTGGCCGTCGAAAAGGAACGCGACCTGTTCAAG | | |
| | CTGATCCTGAAGAAGGAGGACGAGCTGGGGGAC | | |
| | AGAAGCATCATGTTCACCGTGCAGAACGAAGAC | | |
| | (SEQ ID NO: 117) | | |
| FUSE-504 | GACGCTCACAAATCCGAAGTGGCACACAGGTTT | | |
| | AAGGACCTGGGAGAAGAGAACTTCAAAGCCCTG | | |
| | GTGCTGATTGCATTTGCCCAGTATCTGCAGCAG | | |
| | TGTCCCTTTGAGGACCACGTGAAGCTGGTGAAC | | |
| | GAGGTGACCGAATTTGCCAAGACTTGCGTGGCC | | |
| | GACGAGTCCGCAGAGAACTGCGACAAGTCCCTG | | |
| | CATACCCTGTTTGGCGACAAGCTGTGTACAGTG | | |
| | GCCACCCTGCGGGAGACATACGGTGAGATGGCC | | |
| | GACTGCTGCGCCAAGCAGGAACCCGAGAGAAAC | | |
| | GAATGCTTCCTGCAGCACAAGGACGACAACCCC | | |
| | AACCTGCCCCGGCTGGTGAGGCCCGAAGTGGAC | | |
| | GTGATGTGTACCGCCTTTCACGACAACGAAGAG | | |
| | ACATTTCTGAAGAAGTACCTGTACGAGATCGCC | | |
| | AGAAGGCACCCTTACTTTTACGCCCCCGAGCTG | | |
| | CTGTTCTTTGCCAAGAGGTATAAGGCCGCCTTT | | |
| | ACTGAGTGCTGCCAGGCAGCCGACAAAGCCGCC | | |
| | TGCCTGCTGCCCAAGCTGGACGAGCTGCGCGAC | | |
| | GAGGGCAAGGCCTCCTCCGCTAAGCAGAGGCTG | | |
| | AAGTGCGCATCCCTGCAGAAATTTGGCGAGAGA | | |
| | GCCTTCAAGGCATGGGCCGTGGCTAGGCTGTCC | | |
| | CAGAGGTTTCCAAAGGCCGAATTTGCCGAGGTG | | |
| | TCCAAGCTGGTGACAGACCTGACCAAGGTGCAC | | |
| | ACCGAGTGTTGTCACGGAGACTTGCTGGAGTGC | | |
| | GCCGACGACAGGGCCGACCTGGCTAAGTACATC | | |
| | TGTGAGAACCAGGACTCTATCTCCTCCAAGCTG | | |
| | AAGGAGTGCTGCGAGAAGCCCCTGCTGGAGAAA | | |
| | AGCCACTGTATTGCTGAGGTGGAGAACGACGAG | | |
| | ATGCCCGCCGACCTGCCCTCTCTGGCCGCTGAT | | |
| | TTCGTGGAGTCCAAGGACGTGTGTAAGAACTAC | | |
| | GCCGAGGCCAAAGACGTGTTCCTGGGGATGTTT | | |
| | CTGTATGAGTACGCTAGGAGGCACCCCGATTAC | | |
| | TCCGTGGTGCTGCTGCTGAGGCTGGCAAAAACA | | |
| | TACGAAACCACCCTGGAGAAGTGCTGTGCCGCT | | |
| | GCTGACCCCCACGAGTGCTATGCCAAGGTGTTC | | |
| | GACGAGTTCAAACCCCTGGTGGAGGAACCCCAG | | |
| | AACCTGATCAAGCAGAACTGCGAGCTGTTCGAA | | |
| | CAGCTGGGCGAGTACAAATTCCAGAACGCACTG | | |
| | CTGGTGAGGTACACCAAGAAAGTGCCCCAGGTG | | |
| | AGCACCCCCACCCTGGTGGAGGTGTCCAGGAAC | | |
| | CTGGGCAAAGTGGGCAGCAAATGCTGCAAGCAC | | |
| | CCCGAGGCCAAAAGAATGCCCTGTGCAGAGGAC | | |
| | TACCTGAGTGTGGTGCTGAACCAGCTGTGCGTG | | |
| | CTGCACGAGAAGACACCCGTGAGCGACAGGGTG | | |
| | ACCAAGTGTTGTACCGAGTCCCTGGTGAACAGA | | |
| | AGGCCCTGTTTTAGTGCCCTGGAAGTGGATGAG | | |
| | ACTTACGTGCCTAAGGAGTTCAACGCAGAGACA | | |
| | TTCACCTTCCATGCCGATATTTGCACCCTGTCC | | |
| | GAAAAGGAAAGACAGATCAAGAAACAGACCGCC | | |
| | CTGGTGGAGCTGGTGAAACACAAGCCCAAGGCC | | |
| | ACCAAGGAACAGCTGAAAGCCGTGATGGACGAC | | |
| | TTCGCCGCCTTCGTGGAGAAATGCTGCAAGGCC | | |
| | GACGACAAAGAAACCTGCTTCGCAGAGGAAGGA | | |
| | AAGAAACTGGTGGCAGCCAGCCAGGCCGCCCTG | | |
| | GGACTGGGAGATGACGACGACAAGTACTTTGGA | | |
| | AAGCTGGAGAGCAAGCTGAGTGTGATCAGGAAC | | |
| | CTGAACGACCAGGTGCTGTTTATCGACCAGGGC | | |
| | AATAGGCCACTGTTTGAGGATATGACAGATTCC | | |
| | GACGTGAGGGACAACGCTCCCAGAACAATTTTC | | |
| | ATCATCTCTAAGTACGGAGACTCCCAGCCAAGG | | |
| | GGCCTGGCTGTGACCATCTCCGTGAAAGTGGAG | | |
| | AAAATCTCCACACTGTCCGTGGAGAACAAGATC | | |
| | ATCTCCTTCAAGGAAATGAACCCCCCCGACAAC | | |
| | ATCAAGGACACCAAATCCGACATCATCTTCTTC | | |
| | CAGCGCTCCGTGCCCGGACATGATAACAAGGTC | | |
| | CAGTTCGAGTCCAGCAGCTACGAGGGATACTTT | | |
| | CTGGCCGTCGAAAAGGAAAGGGACCTCTTCAAA | | |
| | CTGATCCTGAAGAAGGAGGACGAGCTGGGGGAC | | |
| | AGAAGCATTATGTTCACCGTGCAGAACGAGGAC | | |
| | (SEQ ID NO: 118) | | |
| FUSE-505 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGACGATGACGACAAGTATTTCGGAAAGCTGGAAAGCAAGCTGAGCGTG | |
| | | ATCAGGAACCTGAACGACCAGGTGCTGTTCATCGACCAGGGAAACAGGCCCCTGTTTGAGGA | |
| | | CATGACCGACAGCGACGTGAGGGATAACGCCCCTAGAACAATCTTCATTATCTCCATGTATA | |
| | | AAGACTCCCAGCCAAGGGGAATGGCCGTGACCATTTCCGTGAAGGTCGAGAAAATCTCCACA | |
| | | CTGAGCGTGGAGAACAAGATCATCTCCTTCAAAGAGATGAACCCCCCCGACAACATCAAAGA | |
| | | CACAAAGAGCGACATCATCTTCTTCCAGAGGAGCGTGCCCGGCCATGATAACAAGATGCAGT | |
| | | TCGAAAGCTCCTCCTACGAAGGCTACTTCCTGGCCGTCGAAAAGGAAAGGGACCTGTTCAAA | |
| | | CTGATCCTCAAGAAGGAGGACGAGCTGGGGGACAGAAGCATCATGTTCACCGTGCAGAACGA | |
| | | GGAC (SEQ ID NO: 147) | |
| FUSE-507 | GAGCCTAAATCCGCAGACAAAACACACACATGC | | |
| | CCCCCCTGCCCCGCCCCTGAACTGTTGGGAGGA | | |
| | CCCAGCGTGTTTCTGTTCCCCCCCAAACCCAAG | | |
| | GACACCCTGATGATCTCCAGGACCCCCGAGGTG | | |
| | ACCTGCGTGGTCGTGGACGTCTCCCACGAGGAC | | |
| | CCCGAGGTGAAGTTTAATTGGTACGTGGACGGC | | |
| | GTTGAGGTGCACAACGCCAAGACCAAACCCAGG | | |
| | GAGGAGCAGTACAACAGTACATACAGGGTGGTG | | |
| | TCCGTGCTGACCGTGCTGCACCAGGACTGGCTG | | |
| | AACGGCAAGGAGTACAAGTGCAAGGTGTCCAAC | | |
| | AAAGCACTCCCCGCACCAATTGAAAAAACCATC | | |
| | TCCAAAGCCAAAGGACAGCCCAGGGAACCACAG | | |
| | GTCTACACCCTGCCCCCCTCCAGGGAGGAAATG | | |
| | ACCAAGAACCAGGTCAGCTTAACTTGCCTGGTT | | |
| | AAAGGCTTCTACCCCTCCGACATCGCTGTGGAG | | |
| | TGGGAGTCCAACGGCCAGCCCGAGAACAACTAC | | |
| | AAAACCACACCACCCGTGCTCGACTCCGACGGC | | |
| | TCCTTCTTCCTCTACTCCAAACTGACAGTGGAC | | |
| | AAGTCCAGGTGGCAGCAGGGCAACGTCTTTTCC | | |
| | TGCTCCGTGATGCACGAGGCCCTGCACAACCAC | | |
| | TATACCCAGAAGTCCCTGTCCCTGTCCCCCGGC | | |
| | AAAGCCGCCGAGCCTGTGGAGGACAACGTGATC | | |
| | AACTTTGTGGCCATGAAGTTCATCGACAACACA | | |
| | CTGTACTTCATCGCAGAGGACGACGAGAACGAC | | |
| | GACGACGACAAGTACTTCGGAAAACTGGAGTCC | | |
| | AAGCTGTCCGTGATCAGGAACCTGAATGATCAG | | |
| | GTGCTCTTCATCGACCAGGGCAACAGGCCCCTG | | |
| | TTTGAGGATATGACCGACAGCGACGTGAGGGAC | | |
| | AACGCACCCAGGACAATTTTCATCATTTCTATG | | |
| | TACAAGGACTCCCAGCCCAGGGGCATGGCCGTG | | |
| | ACCATCAGCGTGAAGGTGGAGAAGATCAGCACA | | |
| | CTGAGCGTGGAGAACAAGATCATCTCCTTCAAG | | |
| | GAGATGAACCCCCCCGACAACATCAAAGACACC | | |
| | AAAAGCGATATCATCTTCTTTCAGAGGAGCGTG | | |
| | CCCGGCCACGACAACAAAATGCAGTTCGAGTCC | | |
| | TCCTCCTACGAGGGCTACTTCCTCGCCGTCGAA | | |
| | AAGGAAAGGGACCTGTTCAAGCTGATCCTGAAG | | |
| | AAGGAGGACGAGCTGGGGGACAGAAGCATCATG | | |
| | TTCACCGTGCAGAACGAGGAC (SEQ ID | | |
| | NO: 119) | | |
| FUSE-508 | GAGCCTAAATCCGCAGACAAAACACACACATGC | | |
| | CCCCCCTGCCCCGCCCCTGAACTGTTGGGAGGA | | |
| | CCCAGCGTGTTTCTGTTCCCCCCCAAACCCAAG | | |
| | GACACCCTGATGATCTCCAGGACCCCCGAGGTG | | |
| | ACCTGCGTGGTCGTGGACGTCTCCCACGAGGAC | | |
| | CCCGAGGTGAAGTTTAATTGGTACGTGGACGGC | | |
| | GTTGAGGTGCACAACGCCAAGACCAAACCCAGG | | |
| | GAGGAGCAGTACAACAGTACATACAGGGTGGTG | | |
| | TCCGTGCTGACCGTGCTGCACCAGGACTGGCTG | | |
| | AACGGCAAGGAGTACAAGTGCAAGGTGTCCAAC | | |
| | AAAGCACTCCCCGCACCAATTGAAAAAACCATC | | |
| | TCCAAAGCCAAAGGACAGCCCAGGGAACCACAG | | |
| | GTCTACACCCTGCCCCCCTCCAGGGAGGAAATG | | |
| | ACCAAGAACCAGGTCAGCTTAACTTGCCTGGTT | | |
| | AAAGGCTTCTACCCCTCCGACATCGCTGTGGAG | | |
| | TGGGAGTCCAACGGCCAGCCCGAGAACAACTAC | | |
| | AAAACCACACCACCCGTGCTCGACTCCGACGGC | | |
| | TCCTTCTTCCTCTACTCCAAACTGACAGTGGAC | | |
| | AAGTCCAGGTGGCAGCAGGGCAACGTCTTTTCC | | |
| | TGCTCCGTGATGCACGAGGCCCTGCACAACCAC | | |
| | TATACCCAGAAGTCCCTGTCCCTGTCCCCCGGC | | |
| | AAAGACGACGACGATAAGTACTTCGGCAAACTC | | |
| | GAGAGCAAGTTGTCCGTGATTCGCAACCTGAAC | | |
| | GACCAGGTCCTCTTCATCGACCAGGGCAACCGC | | |
| | CCCCTGTTCGAGGACATGACCGACTCCGACGTG | | |
| | AGGGACAACGCTCCCCGCACCATCTTCATTATC | | |
| | AGTATGTATAAGGATTCCCAGCCCCGCGGCATG | | |
| | GCCGTGACCATCTCCGTGAAGGTGGAGAAGATT | | |
| | TCCACATTGTCCGTGGAGAACAAGATCATCTCC | | |
| | TTCAAAGAGATGAACCCCCCCGACAACATCAAG | | |
| | GACACAAAATCCGACATCATCTTCTTCCAGAGG | | |
| | AGTGTGCCCGGCCACGACAACAAAATGCAGTTC | | |
| | GAATCCAGCTCCTACGAGGGCTACTTCCTCGCC | | |
| | GTCGAAAAGGAAAGGGACCTGTTCAAGCTGATC | | |
| | CTGAAGAAGGAGGATGAGCTGGGGGACAGATCC | | |
| | ATCATGTTCACCGTGCAGAACGAGGAC (SEQ | | |
| | ID NO: 120) | | |
| FUSE-509 | GAGTCTAAGTACGGGCCCCCATGCCCCTCCTGC | | |
| | CCCGCTCCTGAATTTTTGGGCGGCCCCTCCGTT | | |
| | TTTTTGTTTCCCCCCAAGCCCAAGGACACCCTG | | |
| | ATGATTTCCCGCACACCCGAGGTCACCTGCGTC | | |
| | GTCGTCGACGTCTCCCAGGAAGATCCCGAGGTC | | |
| | CAGTTTAATTGGTACGTGGACGGCGTGGAAGTC | | |
| | CACAACGCCAAGACCAAGCCCCGCGAGGAACAG | | |
| | TTCAATTCAACCTACAGGGTGGTGTCCGTCCTC | | |
| | ACCGTGCTGCATCAGGATTGGCTGAACGGCAAG | | |
| | GAATACAAGTGCAAGGTGTCCAACAAAGGCCTC | | |
| | CCCTCCTCTATCGAGAAAACCATCTCCAAAGCC | | |
| | AAAGGCCAGCCCAGGGAACCCCAGGTGTACACC | | |
| | CTGCCCCCCTCCCAGGAAGAAATGACCAAGAAC | | |
| | CAGGTGAGCCTGACATGCCTCGTGAAGGGCTTC | | |
| | TACCCCTCCGACATCGCCGTGGAATGGGAGTCC | | |
| | AACGGCCAGCCCGAAAACAACTACAAAACCACA | | |
| | CCCCCCGTGCTCGACTCCGACGGCTCCTTCTTC | | |
| | CTCTACTCTAGGTTGACCGTGGACAAGTCCAGG | | |
| | TGGCAGGAGGGCAACGTCTTTTCCTGCTCCGTG | | |
| | ATGCACGAGGCCCTCCACAACCACTATACCCAG | | |
| | AAGTCCCTGTCCCTGTCCCTCGGCAAGGCAGCC | | |
| | GAACCTGTGGAGGACAACGTGATCAACTTCGTG | | |
| | GCCATGAAGTTCATCGATAACACCCTGTACTTC | | |
| | ATCGCAGAAGATGATGAAAACGACGACGACGAC | | |
| | AAATACTTCGGCAAACTGGAGTCCAAGCTGAGC | | |
| | GTGATTAGGAACCTGAATGATCAGGTGCTCTTC | | |
| | ATTGACCAGGGCAACAGGCCCCTCTTTGAGGAC | | |
| | ATGACCGACTCCGACGTGAGGGACAACGCCCCC | | |
| | CGCACCATCTTCATCATCTCCATGTACAAGGAC | | |
| | TCCCAGCCCAGGGGCATGGCCGTGACCATTTCC | | |
| | GTGAAGGTGGAGAAAATCTCCACACTGTCTGTG | | |
| | GAGAACAAAATCATCTCCTTCAAAGAGATGAAC | | |
| | CCCCCCGACAACATCAAAGACACAAAAAGCGAT | | |
| | ATCATTTTCTTCCAGAGGTCCGTGCCCGGCCAC | | |
| | GACAACAAAATGCAGTTCGAATCCTCCAGCTAC | | |
| | GAGGGCTACTTCCTGGCCGTCGAAAAGGAAAGG | | |
| | GACCTGTTCAAGCTCATTCTGAAGAAGGAGGAC | | |
| | GAGCTCGGTGACAGGTCCATCATGTTCACCGTC | | |
| | CAGAACGAGGAC (SEQ ID NO: 121) | | |
| FUSE-510 | GAGTCTAAGTACGGGCCCCCATGCCCCTCCTGC | | |
| | CCCGCTCCTGAATTTTTGGGCGGCCCCTCCGTT | | |
| | TTTTTGTTTCCCCCCAAGCCCAAGGACACCCTG | | |
| | ATGATTTCCCGCACACCCGAGGTCACCTGCGTC | | |
| | GTCGTCGACGTCTCCCAGGAAGATCCCGAGGTC | | |
| | CAGTTTAATTGGTACGTGGACGGCGTGGAAGTC | | |
| | CACAACGCCAAGACCAAGCCCCGCGAGGAACAG | | |
| | TTCAATTCAACCTACAGGGTGGTGTCCGTCCTC | | |
| | ACCGTGCTGCATCAGGATTGGCTGAACGGCAAG | | |
| | GAATACAAGTGCAAGGTGTCCAACAAAGGCCTC | | |
| | CCCTCCTCTATCGAGAAAACCATCTCCAAAGCC | | |
| | AAAGGCCAGCCCAGGGAACCCCAGGTGTACACC | | |
| | CTGCCCCCCTCCCAGGAAGAAATGACCAAGAAC | | |
| | CAGGTGAGCCTGACATGCCTCGTGAAGGGCTTC | | |
| | TACCCCTCCGACATCGCCGTGGAATGGGAGTCC | | |
| | AACGGCCAGCCCGAAAACAACTACAAAACCACA | | |
| | CCCCCCGTGCTCGACTCCGACGGCTCCTTCTTC | | |
| | CTCTACTCTAGGTTGACCGTGGACAAGTCCAGG | | |
| | TGGCAGGAGGGCAACGTCTTTTCCTGCTCCGTG | | |
| | ATGCACGAGGCCCTCCACAACCACTATACCCAG | | |
| | AAGTCCCTGTCCCTGTCCCTCGGCAAGGACGAC | | |
| | GACGATAAGTACTTCGGCAAACTGGAGAGCAAG | | |
| | CTGTCCGTGATCCGCAATCTGAACGATCAGGTC | | |
| | CTCTTCATCGACCAGGGCAACCGCCCCCTGTTC | | |
| | GAGGACATGACCGACAGCGACGTGAGGGACAAC | | |
| | GCCCCCAGGACAATCTTCATCATTTCCATGTAC | | |
| | AAGGATAGCCAGCCCAGGGGCATGGCAGTGACC | | |
| | ATCTCCGTGAAAGTGGAGAAGATCAGCACCTTG | | |
| | AGCGTGGAGAACAAGATCATCAGCTTCAAGGAG | | |
| | ATGAACCCCCCCGACAACATCAAGGACACCAAA | | |
| | TCCGACATCATCTTCTTCCAGCGCTCCGTGCCT | | |
| | GGCCACGACAACAAAATGCAGTTCGAGTCCAGC | | |
| | AGCTACGAAGGCTACTTCCTGGCCGTCGAAAAG | | |
| | GAAAGGGACCTGTTCAAGCTGATCCTGAAGAAG | | |
| | GAGGACGAGCTGGGGGACAGGAGCATCATGTTC | | |
| | ACCGTCCAGAACGAAGAC (SEQ ID NO: 122) | | |
| FUSE-516 | (SEQ ID NO: 114) | GAGGTGCAGCTGGTGGAGTCCGGAGGAGGACTGGTGCAGGCTGGAGGAAGCCTGAGGCTGTC | |
| | | CTGCGCTGCTTCCGGAAGGACCTTTAGTTCCTATGCTATGGGTTGGTTTAGGCAGGCCCCTG | |
| | | GAAAGGAGAGGGAGTTTGTGGTGGCCATTAACTGGAGTAGCGGAAGCACCTACTACGCCGAC | |
| | | AGTGTGAAGGGACGGTTCACAATCTCCAGGGACAATGCTAAGAACACAATGTATCTGCAGAT | |
| | | GAACAGCCTGAAGCCTGAGGACACCGCCGTGTATTACTGCGCAGCTGGATACCAGATCAATA | |
| | | GCGGAAACTACAACTTCAAGGACTACGAATACGACTACTGGGGCCAGGGCACACAGGTGACC | |
| | | GTGTCCTCCGAGCCCAAATCTTCTGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAAGCTGCTGAACCTGTGGAAGATAATGTGATCAATTTCGTG | |
| | | GCCATGAAGTTCATTGACAACACCCTGTACTTCATCGCTGAAGATGATGAAAACATCGAGCA | |
| | | GGACTATTTCGGAAAACTGGAAAGCAAACTGAGCGTGATCAGGAACCTGAACGACCAGGTGC | |
| | | TGTTTATCGACCAGGGCAACAGGCCCCTGTTCGAAGATATGACCGACAGCGACGTCAGGGAC | |
| | | AACGCTCCTAGAACAATCTTCATCATCTCCAAGTACGGCGACTCCCAGCCAAGGGGACTGGC | |
| | | TGTGACCATTTCCGTGAAAGTGGAGAAGATCTCCACACTGAGCGTGGAAAAAAAATCATCT | |
| | | CCTTCAAGGAGATGAACCCCCCCGACAACATTAAAGACACCAAAAGCGATATCATCTTCTTC | |
| | | CAGAGGAGTGTGCCCGGCCACGATAACAAAGTGCAGTTCGAGAGCAGCTCCTACGAGGGATA | |
| | | CTTTCTGGCCGTCGAAAAGGAGAGGGACCTGTTCAAGCTGATCCTGAAGAAGGAGGACGAGC | |
| | | TGGGGGACAGATCAATCATGTTCACCGTGCAGAACGAAGAC (SEQ ID NO: 148) | |
| FUSE-517 | (SEQ ID NO: 114) | CAGGTGCAGCTGAAGCAGTCTGGACCAGGACTCGTGCAGCCAAGTCAGTCTCTCTCCATCAC | SEQ |
| | | ATGCACAGTCTCCGGCTTCTCCCTCACAAACTACGGCGTCCACTGGGTGCGACAGTCACCTG | ID |
| | | GAAAAGGCCTCGAATGGCTCGGCGTGATCTGGTCCGGAGGAAATACCGACTACAACACCCCC | NO: 186 |
| | | TTTACCTCCAGGCTGTCCATCAACAAGGATAATTCCAAGTCCCAGGTGTTCTTCAAGATGAA | |
| | | TAGCCTGCAGTCCAACGATACCGCCATCTACTACTGCGCCAGGGCTCTGACATACTACGACT | |
| | | ACGAGTTCGCCTACTGGGGCCAGGGAACACTGGTGACCGTGTCCGCTGCTAGCACCAAGGGC | |
| | | CCATCGGTCTTCCCCCTGGCACCCTCCTCCAAGAGCACCTCTGGGGGCACAGCGGCCCTGGG | |
| | | CTGCCTGGTCAAGGACTACTTCCCCGAACCGGTGACGGTGTCGTGGAACTCAGGCGCCCTGA | |
| | | CCAGCGGCGTGCACACCTTCCCGGCTGTCCTACAGTCCTCAGGACTCTACTCCCTCAGCAGC | |
| | | GTGGTGACCGTGCCCTCCAGCAGCTTGGGCACCCAGACCTACATCTGCAACGTGAATCACAA | |
| | | GCCCAGCAACACCAAGGTGGACAAGAAAGTTGAGCCCAAATCTTGTGACAAAACTCACACAT | |
| | | GCCCACCGTGCCCAGCACCTGAACTCCTGGGGGGACCGTCAGTCTTCCTCTTCCCCCCAAAA | |
| | | CCCAAGGACACCCTCATGATCTCCCGGACCCCCGAGGTCACATGCGTGGTGGTGGACGTGAG | |
| | | CCACGAAGACCCTGAGGTCAAGTTCAACTGGTACGTGGACGGCGTGGAGGTGCATAATGCCA | |
| | | AGACAAAGCCGCGGGAGGAGCAGTACAACAGCACGTACCGTGTGGTCAGCGTCCTCACCGTC | |
| | | CTGCACCAGGACTGGCTGAATGGCAAGGAGTACAAGTGCAAGGTCTCCAACAAAGCCCTCCC | |
| | | AGCCCCCATCGAGAAAACCATCTCCAAAGCCAAAGGGCAGCCCCGAGAACCACAGGTGTACA | |
| | | CCCTGCCCCCATCCCGGGAGGAGATGACCAAGAACCAGGTCAGCCTGTGGTGCCTGGTCAAA | |
| | | GGCTTCTATCCCAGCGACATCGCCGTGGAGTGGGAGAGCAATGGGCAGCCGGAGAACAACTA | |
| | | CAAGACCACGCCTCCCGTGCTGGACTCCGACGGCTCCTTCTTCCTCTACAGCAAGCTCACCG | |
| | | TGGACAAGAGCAGGTGGCAGCAGGGGAACGTCTTCTCATGCTCCGTGATGCATGAGGCTCTG | |
| | | CACAACCACTACACGCAGAAGAGCCTCTCCCTGTCTCCGGGTAAAGCTGCTGAACCTGTGGA | |
| | | AGATAATGTGATCAATTTCGTGGCCATGAAGTTCATTGACAACACCCTGTACTTCATCGCTG | |
| | | AAGATGATGAAAACATCGAGCAGGACTATTTCGGAAAACTGGAAAGCAAACTGAGCGTGATC | |
| | | AGGAACCTGAACGACCAGGTGCTGTTTATCGACCAGGGCAACAGGCCCCTGTTCGAAGATAT | |
| | | GACCGACAGCGACGTCAGGGACAACGCTCCTAGAACAATCTTCATCATCTCCAAGTACGGCG | |
| | | ACTCCCAGCCAAGGGGACTGGCTGTGACCATTTCCGTGAAAGTGGAGAAGATCTCCACACTG | |
| | | AGCGTGGAAAAAAAATCATCTCCTTCAAGGAGATGAACCCCCCCGACAACATTAAAGACAC | |
| | | CAAAAGCGATATCATCTTCTTCCAGAGGAGTGTGCCCGGCCACGATAACAAAGTGCAGTTCG | |
| | | AGAGCAGCTCCTACGAGGGATACTTTCTGGCCGTCGAAAAGGAGAGGGACCTGTTCAAGCTG | |
| | | ATCCTGAAGAAGGAGGACGAGCTGGGGGACAGATCAATCATGTTCACCGTGCAGAACGAAGA | |
| | | C (SEQ ID NO: 149) | |
| FUSE-545 | (SEQ ID NO: 114) | TACTTCGGCAAGCTGGAGTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGATAACG | |
| | | CTCCAAGGACAATCTTCATCATCTCCAAGTATGGAGACTCCCAGCCAAGGGGACTCGCTGTG | |
| | | ACCATTTCCGTGAAGGTGGAGAAGATCAGCACACTCTCCGTGGAAAACAAGATCATCAGCTT | |
| | | CAAAGAGATGAACCCCCCCGACAACATCAAGGACACCAAATCCGACATCATCTTCTTCCAGC | |
| | | GCTCCGTGCCTGGCCATGATAACAAAGTGCAGTTCGAGTCCAGCTCCTACGAGGGATACTTT | |
| | | CTCGCCGTCGAAAAGGAAAGGGACCTGTTCAAGCTCATCCTGAAGAAGGAGGACGAACTGGG | |
| | | CGACAGGTCCATTATGTTCACCGTCCAGAATGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 150) | |
| FUSE-546 | (SEQ ID NO: 114) | TACTTCGGCAAGCTGAAGTCCAAGCTGTCCGTGATCCGGAACCTGAACGACCAGGTGCTGTT | |
| | | CATCGACCAGGGCAACAGGCCACTCTTCGAGGATATGACCGACTCCGACGTGAGGGACAACG | |
| | | CTCCTAGAACCATCTTCATCATCTCCATGTACGGAGACTCCCAGCCAAGGGGAATGGCTGTG | |
| | | ACAATTTCCGTGAAGGTGGAGAAAATTTCCACACTGTCCGTGGAAAACAAGATCATTTCCTT | |
| | | CAAGGAGATGAACCCCCCCGACAACATTAAAGACACCAAGAGCGACATCATCTTCTTCCAGC | |
| | | GCTCTGTGCCCGGCCATGATAACAAGATGCAGTTCGAGTCCAGCTCCTACGAGGGCTACTTT | |
| | | CTGGCCGTCGAAAAGGAAAGGGACCTGTTCAAACTGATCCTGAAGAAGGAGGACGAACTGGG | |
| | | GGACAGGAGCATCATGTTCACCGTGCAGAATGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 151) | |
| FUSE-547 | (SEQ ID NO: 114) | TACTTCGGCAAGCTGCGTTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGACAACG | |
| | | CTCCTAGAACCATCTTCATCATCTCCATGTACGGAGACTCCCAGCCAAGGGGAATGGCTGTG | |
| | | ACAATTTCCGTGAAGGTGGAGAAAATTTCCACACTGTCCGTGGAAAACAAGATCATTTCCTT | |
| | | CAAGGAGATGAACCCCCCCGACAACATTAAAGACACCAAGAGCGACATCATCTTCTTCCAGC | |
| | | GCTCTGTGCCCGGCCATGATAACAAGATGCAGTTCGAGTCCAGCTCCTACGAGGGCTACTTT | |
| | | CTGGCCGTCGAAAAGGAAAGGGACCTGTTCAAACTGATCCTGAAGAAGGAGGACGAACTGGG | |
| | | GGACAGGAGCATCATGTTCACCGTGCAGAATGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 152) | |
| FUSE-555 | CAGGTGCAGCTGAAGCAGTCTGGACCAGGACTC | CAGGTGCAGCTGAAGCAGTCTGGACCAGGACTCGTGCAGCCAAGTCAGTCTCTCTCCATCAC | SEQ |
| | GTGCAGCCAAGTCAGTCTCTCTCCATCACATGC | ATGCACAGTCTCCGGCTTCTCCCTCACAAACTACGGCGTCCACTGGGTGCGACAGTCACCTG | ID |
| | ACAGTCTCCGGCTTCTCCCTCACAAACTACGGC | GAAAAGGCCTCGAATGGCTCGGCGTGATCTGGTCCGGAGGAAATACCGACTACAACACCCCC | NO: 186 |
| | GTCCACTGGGTGCGACAGTCACCTGGAAAAGGC | TTTACCTCCAGGCTGTCCATCAACAAGGATAATTCCAAGTCCCAGGTGTTCTTCAAGATGAA | |
| | CTCGAATGGCTCGGCGTGATCTGGTCCGGAGGA | TAGCCTGCAGTCCAACGATACCGCCATCTACTACTGCGCCAGGGCTCTGACATACTACGACT | |
| | AATACCGACTACAACACCCCCTTTACCTCCAGG | ACGAGTTCGCCTACTGGGGCCAGGGAACACTGGTGACCGTGTCCGCTGCTAGCACCAAGGGC | |
| | CTGTCCATCAACAAGGATAATTCCAAGTCCCAG | CCATCGGTCTTCCCCCTGGCACCCTCCTCCAAGAGCACCTCTGGGGGCACAGCGGCCCTGGG | |
| | GTGTTCTTCAAGATGAATAGCCTGCAGTCCAAC | CTGCCTGGTCAAGGACTACTTCCCCGAACCGGTGACGGTGTCGTGGAACTCAGGCGCCCTGA | |
| | GATACCGCCATCTACTACTGCGCCAGGGCTCTG | CCAGCGGCGTGCACACCTTCCCGGCTGTCCTACAGTCCTCAGGACTCTACTCCCTCAGCAGC | |
| | ACATACTACGACTACGAGTTCGCCTACTGGGGC | GTGGTGACCGTGCCCTCCAGCAGCTTGGGCACCCAGACCTACATCTGCAACGTGAATCACAA | |
| | CAGGGAACACTGGTGACCGTGTCCGCTGCTAGC | GCCCAGCAACACCAAGGTGGACAAGAAAGTTGAGCCCAAATCTTGTGACAAAACTCACACAT | |
| | ACCAAGGGCCCATCGGTCTTCCCCCTGGCACCC | GCCCACCGTGCCCAGCACCTGAACTCCTGGGGGGACCGTCAGTCTTCCTCTTCCCCCCAAAA | |
| | TCCTCCAAGAGCACCTCTGGGGGCACAGCGGCC | CCCAAGGACACCCTCATGATCTCCCGGACCCCCGAGGTCACATGCGTGGTGGTGGACGTGAG | |
| | CTGGGCTGCCTGGTCAAGGACTACTTCCCCGAA | CCACGAAGACCCTGAGGTCAAGTTCAACTGGTACGTGGACGGCGTGGAGGTGCATAATGCCA | |
| | CCGGTGACGGTGTCGTGGAACTCAGGCGCCCTG | AGACAAAGCCGCGGGAGGAGCAGTACAACAGCACGTACCGTGTGGTCAGCGTCCTCACCGTC | |
| | ACCAGCGGCGTGCACACCTTCCCGGCTGTCCTA | CTGCACCAGGACTGGCTGAATGGCAAGGAGTACAAGTGCAAGGTCTCCAACAAAGCCCTCCC | |
| | CAGTCCTCAGGACTCTACTCCCTCAGCAGCGTG | AGCCCCCATCGAGAAAACCATCTCCAAAGCCAAAGGGCAGCCCCGAGAACCACAGGTGTACA | |
| | GTGACCGTGCCCTCCAGCAGCTTGGGCACCCAG | CCCTGCCCCCATCCCGGGAGGAGATGACCAAGAACCAGGTCAGCCTGTGGTGCCTGGTCAAA | |
| | ACCTACATCTGCAACGTGAATCACAAGCCCAGC | GGCTTCTATCCCAGCGACATCGCCGTGGAGTGGGAGAGCAATGGGCAGCCGGAGAACAACTA | |
| | AACACCAAGGTGGACAAGAAAGTTGAGCCCAAA | CAAGACCACGCCTCCCGTGCTGGACTCCGACGGCTCCTTCTTCCTCTACAGCAAGCTCACCG | |
| | TCTTGTGACAAAACTCACACATGCCCACCGTGC | TGGACAAGAGCAGGTGGCAGCAGGGGAACGTCTTCTCATGCTCCGTGATGCATGAGGCTCTG | |
| | CCAGCACCTGAACTCCTGGGGGGACCGTCAGTC | CACAACCACTACACGCAGAAGAGCCTCTCCCTGTCTCCGGGTAAAGGTGGCGGAGGGTCTGG | |
| | TTCCTCTTCCCCCCAAAACCCAAGGACACCCTC | TGGTGGAGGATCCGGGGGAGGAGGTTCAGGAGGCGGGGGAAGTGCTGCTGAACCTGTGGAAG | |
| | ATGATCTCCCGGACCCCCGAGGTCACATGCGTG | ATAATGTGATCAATTTCGTGGCCATGAAGTTCATTGACAACACCCTGTACTTCATCGCTGAA | |
| | GTGGTGGACGTGAGCCACGAAGACCCTGAGGTC | GATGATGAAAACATCGAGCAGGACTATTTCGGAAAACTGGAAAGCAAACTGAGCGTGATCAG | |
| | AAGTTCAACTGGTACGTGGACGGCGTGGAGGTG | GAACCTGAACGACCAGGTGCTGTTTATCGACCAGGGCAACAGGCCCCTGTTCGAAGATATGA | |
| | CATAATGCCAAGACAAAGCCGCGGGAGGAGCAG | CCGACAGCGACGTCAGGGACAACGCTCCTAGAACAATCTTCATCATCTCCAAGTACGGCGAC | |
| | TACAACAGCACGTACCGTGTGGTCAGCGTCCTC | TCCCAGCCAAGGGGACTGGCTGTGACCATTTCCGTGAAAGTGGAGAAGATCTCCACACTGAG | |
| | ACCGTCCTGCACCAGGACTGGCTGAATGGCAAG | CGTGGAAAACAAAATCATCTCCTTCAAGGAGATGAACCCCCCCGACAACATTAAAGACACCA | |
| | GAGTACAAGTGCAAGGTCTCCAACAAAGCCCTC | AAAGCGATATCATCTTCTTCCAGAGGAGTGTGCCCGGCCACGATAACAAAGTGCAGTTCGAG | |
| | CCAGCCCCCATCGAGAAAACCATCTCCAAAGCC | AGCAGCTCCTACGAGGGATACTTTCTGGCCGTCGAAAAGGAGAGGGACCTGTTCAAGCTGAT | |
| | AAAGGGCAGCCCCGAGAACCACAGGTGTACACC | CCTGAAGAAGGAGGACGAGCTGGGGGACAGATCAATCATGTTCACCGTGCAGAACGAAGAC | |
| | CTGCCCCCATCCCGGGAGGAGATGACCAAGAAC | (SEQ ID NO: 153) | |
| | CAGGTCAGCCTGAGCTGCGCCGTCAAAGGCTTC | | |
| | TATCCCAGCGACATCGCCGTGGAGTGGGAGAGC | | |
| | AATGGGCAGCCGGAGAACAACTACAAGACCACG | | |
| | CCTCCCGTGCTGGACTCCGACGGCTCCTTCTTC | | |
| | CTCGTGAGCAAGCTCACCGTGGACAAGAGCAGG | | |
| | TGGCAGCAGGGGAACGTCTTCTCATGCTCCGTG | | |
| | ATGCATGAGGCTCTGCACAACCGCTTCACGCAG | | |
| | AAGAGCCTCTCCCTGTCTCCGGGTAAA (SEQ | | |
| | ID NO: 123) | | |
| FUSE-556 | (SEQ ID NO: 114) | GAAGTGCAGCTGGTGGAAAGCGGAGGAGGATTGGTGCAGGCAGGAGGAAGTCTGAGGCTGAG | |
| | | TTGTGCCGCTAGCGGTAGAACATTTAGTAGTTATGCTATGGGGTGGTTTAGGCAGGCTCCTG | |
| | | GAAAGGAGAGGGAGTTCGTGGTGGCTATCAACTGGAGCAGCGGCTCTACATACTACGCTGAT | |
| | | TCCGTGAAGGGAAGGTTCACAATCTCCAGGGACAATGCTAAGAACACAATGTATCTGCAGAT | |
| | | GAACAGCCTCAAGCCTGAGGACACCGCCGTTTACTATTGCGCCGCTGGATACCAAATCAACA | |
| | | GCGGCAACTACAACTTCAAGGACTATGAGTATGACTACTGGGGCCAGGGCACACAGGTGACA | |
| | | GTTTCATCCGAGCCCAAATCTTCTGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAA (SEQ ID NO: 154) | |
| FUSE-557 | (SEQ ID NO: 114) | GAGCCCAAATCTTCTGACAAAACTCACACATGCCCACCGTGCCCAGCACCTGAACTCCTGGG | SEQ |
| | | GGGACCGTCAGTCTTCCTCTTCCCCCCAAAACCCAAGGACACCCTCATGATCTCCCGGACCC | ID |
| | | CCGAGGTCACATGCGTGGTGGTGGACGTGAGCCACGAAGACCCTGAGGTCAAGTTCAACTGG | NO: 186 |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGAGCTGCGCCGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCGTGAGCAAGCTCACCGTGGACAAGAGCAGGTGGCAGCAGGGGAACGT | |
| | | CTTCTCATGCTCCGTGATGCATGAGGCTCTGCACAACCGGTTCACGCAGAAGAGCCTCTCCC | |
| | | TGTCTCCGGGTAAA (SEQ ID NO: 288) | |
| FUSE-583 | (SEQ ID NO: 114) | TACTTCGGCAAGCTGGCTTCCAAGCTGTCCGTGATCAGGAACCTGAACGATCAGGTGCTGTT | |
| | | CATCGATCAGGGCAACAGGCCCCTGTTCGAGGATATGACCGACTCCGATGTGCGCGACAACG | |
| | | CACCTAGAACCATCTTCATCATCTCTATGTACGCCGATTCCCAGCCAAGGGGCATGGCTGTG | |
| | | ACAATTTCCGTGAAGGTGGAGAAGATCTCCACCCTGTCCGTGGAAAACAAAATCATTTCCTT | |
| | | CAAGGAGATGAACCCCCCCGACAACATCAAGGACACTAAAAGCGACATCATCTTCTTCCAGA | |
| | | GGAGCGTGCCCGGCCATGATAACAAAATGCAGTTCGAATCCAGCTCCTACGAAGGCTACTTC | |
| | | CTCGCCGTCGAAAAGGAAAGGGACCTGTTCAAACTGATCCTCAAGAAGGAGGACGAGTTGGG | |
| | | GGACAGATCCATTATGTTCACCGTTCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 155) | |
| FUSE-584 | (SEQ ID NO: 114) | TACTTCGGCAAGCTGCACTCCAAGCTGTCCGTGATCCGGAACCTGAACGACCAGGTGCTCTT | |
| | | CATCGACCAGGGCAACAGGCCTCTCTTCGAGGATATGACCGACTCCGACGTGCGCGATAACG | |
| | | CTCCTAGAACCATCTTCATCATCTCCATGTACGGCGACTCCCAGCCCAGAGGAATGGCTGTG | |
| | | ACAATTTCCGTGAAGGTGGAGAAAATCTCCACCCTGTCCGTGGAGAACAAGATCATCTCCTT | |
| | | CAAAGAGATGAACCCCCCCGACAACATCAAAGACACCAAGAGCGACATCATCTTTTTCCAGC | |
| | | GCTCTGTCCCCGGCCACGATAATAAAATGCAGTTCGAATCCAGCTCCTACGAAGGCTACTTC | |
| | | CTCGCCGTCGAAAAGGAAAGGGACCTCTTCAAGTTGATCCTGAAGAAGGAGGACGAACTGGG | |
| | | GGACAGGAGCATCATGTTCACCGTGCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 156) | |
| FUSE-585 | (SEQ ID NO: 114) | TACTTCGGCAAGCTGCGTTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGACAACG | |
| | | CTCCTAGAACCATCTTCATCATCTCCATGTACGAAGATTCTCAACCCAGGGGCATGGCCGTG | |
| | | ACAATTTCCGTTAAGGTGGAGAAAATCTCCACACTGTCCGTGGAAAACAAGATCATCTCCTT | |
| | | CAAAGAGATGAACCCCCCCGACAACATTAAAGACACAAAAAGCGATATCATCTTCTTCCAGA | |
| | | GGAGCGTGCCCGGGCATGATAACAAGATGCAGTTCGAAAGCTCCTCCTACGAAGGCTACTTC | |
| | | CTGGCCGTCGAAAAGGAAAGGGACCTGTTCAAACTGATCCTGAAGAAGGAGGATGAATTGGG | |
| | | CGACAGGTCCATTATGTTCACAGTGCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 157) | |
| FUSE-586 | (SEQ ID NO: 114) | TACTTCGGCAAGCTGCAGTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGATAACG | |
| | | CTCCAAGGACCATCTTCATCATCTCCATGTACGCCGACTCCCAGCCAAGGGGAATGGCTGTG | |
| | | ACAATCTCCGTGAAAGTGGAGAAGATTAGCACCCTGTCCGTGGAAAACAAGATCATCTCCTT | |
| | | CAAAGAGATGAACCCCCCCGACAACATCAAAGACACCAAATCCGACATCATCTTCTTCCAGC | |
| | | GCTCTGTGCCTGGGCACGATAACAAAATGCAGTTCGAAAGCTCCTCCTACGAGGGATACTTC | |
| | | CTCGCCGTCGAAAAGGAAAGGGACCTGTTCAAGCTGATCCTGAAGAAGGAGGACGAGCTGGG | |
| | | GGACAGAAGCATCATGTTCACCGTTCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 158) | |
| FUSE-587 | (SEQ ID NO: 114) | GAGCCCAAATCTTCTGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCCGTGGAAGATAATGTGATCAATTTCGTGGCCATGAAG | |
| | | TTCATTGACAACACCCTGTACTTCATCGCCGAAGATGATGAAAACGGAGGAGGAGGAAAGCC | |
| | | ACTGGGATTGCAGGCCAGGGTGGTGGGAGGAGGAGGCACAATCGAACAGGACTACTTCGGAA | |
| | | AACTGGAATCCAAACTGAGCGTGATCAGGAACCTGAACGACCAGGTGCTGTTCATCGACCAG | |
| | | GGCAATCGCCCCCTCTTTGAGGACATGACCGACTCCGACGTCCGGGACAACGCTCCCAGGAC | |
| | | CATCTTCATCATCTCCATGTACAAAGACTCCCAGCCAAGGGGCATGGCCGTGACCATCTCCG | |
| | | TCAAGGTGGAGAAGATCTCCACCCTGTCCGTGGAAAACAAGATCATCTCCTTCAAAGAGATG | |
| | | AACCCCCCCGACAACATCAAAGACACAAAATCCGACATCATCTTCTTCCAGCGCTCCGTGCC | |
| | | TGGGCACGACAACAAAATGCAGTTCGAATCCTCCTCCTACGAGGGCTACTTCCTCGCCGTCG | |
| | | AAAAAGAAAGAGACTTGTTCAAGCTCATCCTCAAGAAGGAGGACGAGCTCGGGGACAGGTCA | |
| | | ATCATGTTCACCGTGCAGAACGAGGAC (SEQ ID NO: 159) | |
| FUSE-599 | (SEQ ID NO: 114) | TACTTCGGCAAGCTGAAGTCCAAGCTGTCCGTGATCCGGAACCTGAACGACCAGGTGCTGTT | |
| | | CATCGACCAGGGCAACAGGCCACTCTTCGAGGATATGACCGACTCCGACGTGAGGGACAACG | |
| | | CTCCTAGAACCATCTTCATCATCTCCCACTACGGCGACTCCCAGCCAAGAGGAATGGCTGTT | |
| | | ACAATTTCCGTGAAAGTGGAGAAAATCTCCACCCTGTCCGTTGAGAACAAGATCATCTCCTT | |
| | | CAAGGAGATGAACCCCCCCGACAACATCAAGGACACCAAGAGCGACATCATCTTCTTCCAGC | |
| | | GCTCTGTGCCCGGCCATGATAACAAAATGCAGTTCGAAAGCTCCTCCTACGAGGGCTACTTC | |
| | | CTCGCAGTCGAAAAAGAAAGGGACCTGTTCAAGTTGATCCTGAAGAAGGAGGACGAACTGGG | |
| | | GGACAGGAGCATCATGTTCACCGTGCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 160) | |
| FUSE-600 | (SEQ ID NO: 114) | TACTTCGGCAAGCTGAAGTCCAAGCTGTCCGTGATCCGGAACCTGAACGACCAGGTGCTGTT | |
| | | CATCGACCAGGGCAACAGGCCACTCTTCGAGGATATGACCGACTCCGACGTGAGGGACAACG | |
| | | CTCCTAGAACCATCTTCATCATCTCCAAGTACGGAGACTCCCAGCCAAGGGGAATGGCTGTT | |
| | | ACAATTTCCGTGAAGGTGGAGAAAATCTCCACCCTGTCCGTGGAGAACAAGATCATCAGCTT | |
| | | CAAAGAGATGAACCCCCCCGACAACATCAAAGACACCAAGAGCGACATCATCTTCTTCCAGC | |
| | | GCTCTGTGCCCGGTCACGATAACAAAATGCAGTTCGAATCCAGCTCCTACGAGGGCTACTTC | |
| | | CTCGCAGTCGAAAAGGAAAGGGACCTCTTCAAGTTGATCCTGAAGAAGGAGGACGAGCTGGG | |
| | | GGACAGATCCATCATGTTCACCGTTCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 161) | |
| FUSE-601 | (SEQ ID NO: 114) | TACTTCGGCAAGCTGGAGTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGATAACG | |
| | | CTCCAAGGACAATCTTCATCATCTCCCACTACGCCGACTCCCAGCCAAGAGGAATCGCTGTT | |
| | | ACCATCTCCGTGAAGGTGGAGAAAATTAGCACACTGAGCGTGGAGAACAAGATCATCTCCTT | |
| | | CAAGGAGATGAACCCCCCCGACAACATCAAGGACACCAAAAGCGACATCATCTTCTTCCAGC | |
| | | GCAGCGTGCCCGGACATGATAACAAAATCCAGTTCGAATCCTCCAGCTACGAGGGCTACTTT | |
| | | CTGGCCGTCGAAAAGGAAAGAGACTTGTTCAAGTTGATCCTGAAGAAGGAGGACGAGCTGGG | |
| | | GGACAGAAGCATCATGTTCACCGTTCAGAACGAAGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 162) | |
| FUSE-602 | (SEQ ID NO: 114) | TACTTCGGCAAGCTGGAGTCCAAGCTGTCCGTGATCCGGAACCTGAACGACCAGGTGCTGTT | |
| | | CATCGACCAGGGCAACAGGCCTCTCTTCGAGGATATGACCGACTCCGACGTGAGGGATAACG | |
| | | CTCCAAGGACAATCTTCATCATCTCCAAGTATGCCGACTCCCAGCCAAGGGGAATCGCTGTG | |
| | | ACAATTTCCGTGAAAGTGGAGAAGATCTCCACCCTGTCCGTGGAAAACAAGATCATCTCCTT | |
| | | CAAAGAGATGAACCCCCCCGACAACATCAAGGACACCAAATCCGACATCATCTTCTTCCAGC | |
| | | GCTCTGTGCCTGGGCACGATAACAAAATCCAGTTCGAATCCAGCTCCTACGAGGGCTACTTT | |
| | | CTCGCCGTCGAAAAGGAAAGGGACCTGTTCAAACTCATCCTGAAGAAGGAGGACGAACTGGG | |
| | | AGACAGATCCATCATGTTCACAGTGCAGAACGAGGACGAGCCCAAATCTTCTGACAAAACTC | |
| | | ACACATGCCCACCGTGCCCAGCACCTGAACTCCTGGGGGGACCGTCAGTCTTCCTCTTCCCC | |
| | | CCAAAACCCAAGGACACCCTCATGATCTCCCGGACCCCCGAGGTCACATGCGTGGTGGTGGA | |
| | | CGTGAGCCACGAAGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAAGC | |
| | | CCTCCCAGCCCCCATCGAGAAAACCATCTCCAAAGCCAAAGGGCAGCCCCGAGAACCACAGG | |
| | | TGTACACCCTGCCCCCATCCCGGGAGGAGATGACCAAGAACCAGGTCAGCCTGTGGTGCCTG | |
| | | GTCAAAGGCTTCTATCCCAGCGACATCGCCGTGGAGTGGGAGAGCAATGGGCAGCCGGAGAA | |
| | | CAACTACAAGACCACGCCTCCCGTGCTGGACTCCGACGGCTCCTTCTTCCTCTACAGCAAGC | |
| | | TCACCGTGGACAAGAGCAGGTGGCAGCAGGGGAACGTCTTCTCATGCTCCGTGATGCATGAG | |
| | | GCTCTGCACAACCACTACACGCAGAAGAGCCTCTCCCTGTCTCCGGGTAAA (SEQ ID | |
| | | NO: 163) | |
| FUSE-624 | (SEQ ID NO: 123) | CAGGTGCAGCTGAAGCAGTCTGGACCAGGACTCGTGCAGCCAAGTCAGTCTCTCTCCATCAC | SEQ |
| | | ATGCACAGTCTCCGGCTTCTCCCTCACAAACTACGGCGTCCACTGGGTGCGACAGTCACCTG | ID |
| | | GAAAAGGCCTCGAATGGCTCGGCGTGATCTGGTCCGGAGGAAATACCGACTACAACACCCCC | NO: 186 |
| | | TTTACCTCCAGGCTGTCCATCAACAAGGATAATTCCAAGTCCCAGGTGTTCTTCAAGATGAA | |
| | | TAGCCTGCAGTCCAACGATACCGCCATCTACTACTGCGCCAGGGCTCTGACATACTACGACT | |
| | | ACGAGTTCGCCTACTGGGGCCAGGGAACACTGGTGACCGTGTCCGCTGCTAGCACCAAGGGC | |
| | | CCATCGGTCTTCCCCCTGGCACCCTCCTCCAAGAGCACCTCTGGGGGCACAGCGGCCCTGGG | |
| | | CTGCCTGGTCAAGGACTACTTCCCCGAACCGGTGACGGTGTCGTGGAACTCAGGCGCCCTGA | |
| | | CCAGCGGCGTGCACACCTTCCCGGCTGTCCTACAGTCCTCAGGACTCTACTCCCTCAGCAGC | |
| | | GTGGTGACCGTGCCCTCCAGCAGCTTGGGCACCCAGACCTACATCTGCAACGTGAATCACAA | |
| | | GCCCAGCAACACCAAGGTGGACAAGAAAGTTGAGCCCAAATCTTGTGACAAAACTCACACAT | |
| | | GCCCACCGTGCCCAGCACCTGAACTCCTGGGGGGACCGTCAGTCTTCCTCTTCCCCCCAAAA | |
| | | CCCAAGGACACCCTCATGATCTCCCGGACCCCCGAGGTCACATGCGTGGTGGTGGACGTGAG | |
| | | CCACGAAGACCCTGAGGTCAAGTTCAACTGGTACGTGGACGGCGTGGAGGTGCATAATGCCA | |
| | | AGACAAAGCCGCGGGAGGAGCAGTACAACAGCACGTACCGTGTGGTCAGCGTCCTCACCGTC | |
| | | CTGCACCAGGACTGGCTGAATGGCAAGGAGTACAAGTGCAAGGTCTCCAACAAAGCCCTCCC | |
| | | AGCCCCCATCGAGAAAACCATCTCCAAAGCCAAAGGGCAGCCCCGAGAACCACAGGTGTACA | |
| | | CCCTGCCCCCATCCCGGGAGGAGATGACCAAGAACCAGGTCAGCCTGTGGTGCCTGGTCAAA | |
| | | GGCTTCTATCCCAGCGACATCGCCGTGGAGTGGGAGAGCAATGGGCAGCCGGAGAACAACTA | |
| | | CAAGACCACGCCTCCCGTGCTGGACTCCGACGGCTCCTTCTTCCTCTACAGCAAGCTCACCG | |
| | | TGGACAAGAGCAGGTGGCAGCAGGGGAACGTCTTCTCATGCTCCGTGATGCATGAGGCTCTG | |
| | | CACAACCACTACACGCAGAAGAGCCTCTCCCTGTCTCCGGGTAAAGGTGGCGGAGGGTCTGG | |
| | | TGGTGGAGGATCCGGGGGAGGAGGTTCAGGAGGCGGGGGAAGTGCTGCTGAACCTGTGGAAG | |
| | | ATAATGTGATCAATTTTGTGGCCATGAAATTTATTGACAACACCCTGTATTTCATCGCCGAA | |
| | | GATGATGAAAACGGCGGAGGAGGAAAACCCCTGGGACTGCAGGCTAGAGTGGTGGGAGGAGG | |
| | | AGGAACCATCGAGCAGGACTACTTCGGGAAACTGGAATCTAAGCTGTCAGTTATCAGGAACC | |
| | | TGAACGATCAGGTGCTGTTCATTGACCAGGGCAACAGGCCCTTGTTTGAGGATATGACAGAC | |
| | | AGCGACGTGAGGGACAACGCTCCCAGAACCATCTTCATTATCTCCAAGTACGGAGACAGCCA | |
| | | GCCCAGGGGATTGGCTGTTACCATCTCCGTGAAAGTGGAGAAGATTTCCACCTTGTCAGTGG | |
| | | AGAACAAAATCATCTCCTTCAAAGAGATGAACCCCCCCGACAACATCAAGGACACCAAGTCC | |
| | | GACATCATCTTCTTCCAGCGCTCTGTGCCTGGGCACGACAACAAAGTCCAGTTCGAGAGCTC | |
| | | CAGCTACGAGGGCTACTTTCTGGCCGTCGAGAAGGAACGCGACCTGTTCAAACTCATCCTGA | |
| | | AAAAGGAGGACGAACTGGGAGACAGGTCCATCATGTTCACCGTGCAGAATGAAGAC (SEQ | |
| | | ID NO: 164) | |
| FUSE-625 | GAAGTGCAGCTGGTGGAGAGCGGAGGAGGATTG | GAAGTGCAGCTGGTGGAGAGCGGAGGAGGATTGGTGCAGCCTGGAGGAAGCCTGAGGCTGAG | SEQ |
| | GTGCAGCCTGGAGGAAGCCTGAGGCTGAGTTGT | TTGTGCCGCTAGTGGGTTTAATATCAAGGACACATACATCCACTGGGTGAGGCAGGCCCCCG | ID |
| | GCCGCTAGTGGGTTTAATATCAAGGACACATAC | GAAAAGGATTGGAGTGGGTGGCACGGATTTACCCCACCAATGGATACACAAGGTACGCCGAC | NO: 187 |
| | ATCCACTGGGTGAGGCAGGCCCCCGGAAAAGGA | TCCGTGAAGGGCAGGTTCACCATCTCCGCTGACACAAGCAAGAACACAGCCTACCTGCAGAT | |
| | TTGGAGTGGGTGGCACGGATTTACCCCACCAAT | GAACTCCCTGAGGGCAGAAGATACTGCTGTGTACTATTGCTCCAGGTGGGGAGGAGACGGAT | |
| | GGATACACAAGGTACGCCGACTCCGTGAAGGGC | TCTATGCAATGGACTACTGGGGCCAGGGAACACTGGTGACCGTGTCTAGCGCTAGCACCAAG | |
| | AGGTTCACCATCTCCGCTGACACAAGCAAGAAC | GGCCCATCGGTCTTCCCCCTGGCACCCTCCTCCAAGAGCACCTCTGGGGGCACAGCGGCCCT | |
| | ACAGCCTACCTGCAGATGAACTCCCTGAGGGCA | GGGCTGCCTGGTCAAGGACTACTTCCCCGAACCGGTGACGGTGTCGTGGAACTCAGGCGCCC | |
| | GAAGATACTGCTGTGTACTATTGCTCCAGGTGG | TGACCAGCGGCGTGCACACCTTCCCGGCTGTCCTACAGTCCTCAGGACTCTACTCCCTCAGC | |
| | GGAGGAGACGGATTCTATGCAATGGACTACTGG | AGCGTGGTGACCGTGCCCTCCAGCAGCTTGGGCACCCAGACCTACATCTGCAACGTGAATCA | |
| | GGCCAGGGAACACTGGTGACCGTGTCTAGCGCT | CAAGCCCAGCAACACCAAGGTGGACAAGAAAGTTGAGCCCAAATCTTGTGACAAAACTCACA | |
| | AGCACCAAGGGCCCATCGGTCTTCCCCCTGGCA | CATGCCCACCGTGCCCAGCACCTGAACTCCTGGGGGGACCGTCAGTCTTCCTCTTCCCCCCA | |
| | CCCTCCTCCAAGAGCACCTCTGGGGGCACAGCG | AAACCCAAGGACACCCTCATGATCTCCCGGACCCCCGAGGTCACATGCGTGGTGGTGGACGT | |
| | GCCCTGGGCTGCCTGGTCAAGGACTACTTCCCC | GAGCCACGAAGACCCTGAGGTCAAGTTCAACTGGTACGTGGACGGCGTGGAGGTGCATAATG | |
| | GAACCGGTGACGGTGTCGTGGAACTCAGGCGCC | CCAAGACAAAGCCGCGGGAGGAGCAGTACAACAGCACGTACCGTGTGGTCAGCGTCCTCACC | |
| | CTGACCAGCGGCGTGCACACCTTCCCGGCTGTC | GTCCTGCACCAGGACTGGCTGAATGGCAAGGAGTACAAGTGCAAGGTCTCCAACAAAGCCCT | |
| | CTACAGTCCTCAGGACTCTACTCCCTCAGCAGC | CCCAGCCCCCATCGAGAAAACCATCTCCAAAGCCAAAGGGCAGCCCCGAGAACCACAGGTGT | |
| | GTGGTGACCGTGCCCTCCAGCAGCTTGGGCACC | ACACCCTGCCCCCATCCCGGGAGGAGATGACCAAGAACCAGGTCAGCCTGTGGTGCCTGGTC | |
| | CAGACCTACATCTGCAACGTGAATCACAAGCCC | AAAGGCTTCTATCCCAGCGACATCGCCGTGGAGTGGGAGAGCAATGGGCAGCCGGAGAACAA | |
| | AGCAACACCAAGGTGGACAAGAAAGTTGAGCCC | CTACAAGACCACGCCTCCCGTGCTGGACTCCGACGGCTCCTTCTTCCTCTACAGCAAGCTCA | |
| | AAATCTTGTGACAAAACTCACACATGCCCACCG | CCGTGGACAAGAGCAGGTGGCAGCAGGGGAACGTCTTCTCATGCTCCGTGATGCATGAGGCT | |
| | TGCCCAGCACCTGAACTCCTGGGGGGACCGTCA | CTGCACAACCACTACACGCAGAAGAGCCTCTCCCTGTCTCCGGGTAAAGGTGGCGGAGGGTC | |
| | GTCTTCCTCTTCCCCCCAAAACCCAAGGACACC | TGGTGGTGGAGGATCCGGGGGAGGAGGTTCAGGAGGCGGGGGAAGTGCTGCTGAACCTGTGG | |
| | CTCATGATCTCCCGGACCCCCGAGGTCACATGC | AAGATAATGTGATCAATTTTGTGGCCATGAAATTTATTGACAACACCCTGTATTTCATCGCC | |
| | GTGGTGGTGGACGTGAGCCACGAAGACCCTGAG | GAAGATGATGAAAACGGCGGAGGAGGAAAACCCCTGGGACTGCAGGCTAGAGTGGTGGGAGG | |
| | GTCAAGTTCAACTGGTACGTGGACGGCGTGGAG | AGGAGGAACCATCGAGCAGGACTACTTCGGGAAACTGGAATCTAAGCTGTCAGTTATCAGGA | |
| | GTGCATAATGCCAAGACAAAGCCGCGGGAGGAG | ACCTGAACGATCAGGTGCTGTTCATTGACCAGGGCAACAGGCCCTTGTTTGAGGATATGACA | |
| | CAGTACAACAGCACGTACCGTGTGGTCAGCGTC | GACAGCGACGTGAGGGACAACGCTCCCAGAACCATCTTCATTATCTCCAAGTACGGAGACAG | |
| | CTCACCGTCCTGCACCAGGACTGGCTGAATGGC | CCAGCCCAGGGGATTGGCTGTTACCATCTCCGTGAAAGTGGAGAAGATTTCCACCTTGTCAG | |
| | AAGGAGTACAAGTGCAAGGTCTCCAACAAAGCC | TGGAGAACAAAATCATCTCCTTCAAAGAGATGAACCCCCCCGACAACATCAAGGACACCAAG | |
| | CTCCCAGCCCCCATCGAGAAAACCATCTCCAAA | TCCGACATCATCTTCTTCCAGCGCTCTGTGCCTGGGCACGACAACAAAGTCCAGTTCGAGAG | |
| | GCCAAAGGGCAGCCCCGAGAACCACAGGTGTAC | CTCCAGCTACGAGGGCTACTTTCTGGCCGTCGAGAAGGAACGCGACCTGTTCAAACTCATCC | |
| | ACCCTGCCCCCATCCCGGGAGGAGATGACCAAG | TGAAAAAGGAGGACGAACTGGGAGACAGGTCCATCATGTTCACCGTGCAGAATGAAGAC | |
| | AACCAGGTCAGCCTGAGCTGCGCCGTCAAAGGC | (SEQ ID NO: 165) | |
| | TTCTATCCCAGCGACATCGCCGTGGAGTGGGAG | | |
| | AGCAATGGGCAGCCGGAGAACAACTACAAGACC | | |
| | ACGCCTCCCGTGCTGGACTCCGACGGCTCCTTC | | |
| | TTCCTCGTGAGCAAGCTCACCGTGGACAAGAGC | | |
| | AGGTGGCAGCAGGGGAACGTCTTCTCATGCTCC | | |
| | GTGATGCATGAGGCTCTGCACAACCGCTTCACG | | |
| | CAGAAGAGCCTCTCCCTGTCTCCGGGTAAA | | |
| | (SEQ ID NO: 124) | | |
| FUSE-626 | (SEQ ID NO: 114) | GAAGTGCAGCTGGTGGAGAGCGGAGGAGGATTGGTGCAGGCAGGAGGAAGCCTGAGGCTGAG | |
| | | TTGTGCCGCTAGTGGAAGGACATTTAGTTCCTATGCAATGGGGTGGTTCAGGCAGGCCCCAG | |
| | | GAAAGGAAAGGGAGTTTGTGGTGGCCATCAACTGGAGTAGCGGATCTACATACTACGCTGAT | |
| | | TCTGTGAAGGGCAGGTTCACCATCAGCCGGGACAATGCTAAGAACACCATGTACCTGCAGAT | |
| | | GAACAGCCTGAAGCCTGAGGACACCGCCGTGTACTATTGCGCCGCTGGATATCAGATCAATT | |
| | | CCGGCAACTACAACTTCAAGGACTACGAGTATGACTACTGGGGCCAGGGCACACAGGTGACA | |
| | | GTGTCATCCGAACCTAAATCCTCCGACAAAACCCACACCTGCCCCCCTTGCCCTGCTCCTGA | |
| | | ATTGCTGGGAGGACCCTCCGTGTTTCTGTTTCCCCCCAAACCCAAGGACACCCTGATGATCT | |
| | | CCAGGACCCCCGAGGTGACCTGCGTTGTTGTGGACGTGTCCCACGAGGACCCTGAGGTTAAA | |
| | | TTTAACTGGTACGTCGACGGCGTCGAGGTCCACAACGCAAAAACCAAACCCCGCGAGGAACA | |
| | | GTATAACAGTACCTACAGAGTGGTGTCCGTTCTCACCGTGCTGCACCAGGATTGGCTGAACG | |
| | | GCAAGGAGTATAAGTGTAAAGTGTCCAACAAAGCCCTGCCCGCCCCCATTGAGAAAACTATT | |
| | | TCTAAAGCCAAAGGCCAGCCCAGGGAACCACAGGTTTACACACTGCCCCCCTCCAGAGAAGA | |
| | | AATGACCAAGAACCAGGTGAGCCTGTGGTGCCTCGTCAAAGGATTCTACCCCTCCGACATCG | |
| | | CCGTGGAGTGGGAATCAAATGGCCAGCCCGAGAACAACTACAAAACCACCCCCCCCGTGCTC | |
| | | GACTCCGATGGATCTTTCTTCCTGTACTCCAAGTTGACCGTGGACAAGTCCAGGTGGCAGCA | |
| | | GGGAAACGTGTTCAGCTGCTCCGTGATGCACGAGGCCCTGCATAATCACTATACACAGAAGA | |
| | | GTTTGTCCCTGTCTCCTGGCAAGGCTGCTGAACCTGTGGAAGATAATGTGATCAATTTTGTG | |
| | | GCCATGAAATTTATTGACAACACCCTGTATTTCATCGCCGAAGATGATGAAAACGGCGGAGG | |
| | | ATCTTATTTTGGAAAGCTGGAATCCAAATTGAGCGTGATCAGGAACCTGAACGATCAGGTGC | |
| | | TGTTTATCGACCAGGGCAACAGGCCCCTCTTTGAGGACATGACCGACAGTGACGTGAGGGAC | |
| | | AACGCCCCTAGGACCATCTTCATCATCTCCAAGTACGGCGATTCCCAGCCCAGGGGACTGGC | |
| | | TGTTACAATTTCCGTGAAGGTGGAGAAGATTTCCACCCTGAGCGTGGAGAACAAGATCATTT | |
| | | CCTTCAAAGAGATGAACCCCCCCGACAACATCAAGGACACCAAAAGCGATATCATTTTCTTC | |
| | | CAGCGTAGCGTGCCCGGCCACGATAACAAAGTGCAGTTCGAATCCTCCTCCTACGAGGGCTA | |
| | | CTTCCTGGCCGTCGAAAAGGAAAGGGACCTGTTCAAGTTGATCCTGAAGAAGGAGGACGAAT | |
| | | TGGGGGACAGAAGCATCATGTTCACCGTGCAGAACGAGGAC (SEQ ID NO: 289) | |
| FUSE-627 | GAACCTAAATCCTCCGACAAAACCCACACCTGC | GAAGTGCAGCTGGTGGAGAGCGGAGGAGGATTGGTGCAGGCAGGAGGAAGCCTGAGGCTGAG | |
| | CCCCCTTGCCCTGCTCCTGAAGCTGCTGGAGGA | TTGTGCCGCTAGTGGAAGGACATTTAGTTCCTATGCAATGGGGTGGTTCAGGCAGGCCCCAG | |
| | CCTTCCGTGTTTCTGTTTCCCCCCAAGCCCAAG | GAAAGGAAAGGGAGTTTGTGGTGGCCATCAACTGGAGTAGCGGATCTACATACTACGCTGAT | |
| | GACACCCTCATGATCTCCAGGACCCCCGAGGTC | TCTGTGAAGGGCAGGTTCACCATCAGCCGGGACAATGCTAAGAACACCATGTACCTGCAGAT | |
| | ACCTGCGTTGTTGTGGACGTGTCCCACGAAGAT | GAACAGCCTGAAGCCTGAGGACACCGCCGTGTACTATTGCGCCGCTGGATATCAGATCAATT | |
| | CCTGAAGTTAAATTTAACTGGTACGTGGACGGC | CCGGCAACTACAACTTCAAGGACTACGAGTATGACTACTGGGGCCAGGGCACACAGGTGACA | |
| | GTGGAAGTGCACAACGCCAAGACAAAACCCCGC | GTGTCATCCGAACCTAAATCCTCCGACAAAACCCACACCTGCCCCCCTTGCCCTGCTCCTGA | |
| | GAGGAACAGTATAACTCCACCTACAGGGTGGTG | AGCTGCTGGAGGACCTTCCGTGTTTCTGTTTCCCCCCAAGCCCAAGGACACCCTCATGATCT | |
| | AGCGTGCTGACAGTGCTGCACCAGGACTGGCTG | CCAGGACCCCCGAGGTCACCTGCGTTGTTGTGGACGTGTCCCACGAAGATCCTGAAGTTAAA | |
| | AACGGCAAGGAATACAAGTGCAAGGTGTCCAAC | TTTAACTGGTACGTGGACGGCGTGGAAGTGCACAACGCCAAGACAAAACCCCGCGAGGAACA | |
| | AAGGCCCTGGGCGCTCCTATTGAGAAAACTATT | GTATAACTCCACCTACAGGGTGGTGAGCGTGCTGACAGTGCTGCACCAGGACTGGCTGAACG | |
| | TCTAAAGCCAAGGGACAGCCCCGGGAGCCACAG | GCAAGGAATACAAGTGCAAGGTGTCCAACAAGGCCCTGGGCGCTCCTATTGAGAAAACTATT | |
| | GTTTACACACTGCCTCCCTCCAGGGAAGAGATG | TCTAAAGCCAAGGGACAGCCCCGGGAGCCACAGGTTTACACACTGCCTCCCTCCAGGGAAGA | |
| | ACCAAGAACCAGGTCAGCCTGTCCTGCGCTGTT | GATGACCAAGAACCAGGTCAGCCTGTGGTGCCTGGTGAAGGGATTCTACCCCTCCGACATCG | |
| | AAGGGCTTCTACCCCTCCGACATCGCCGTGGAA | CCGTGGAATGGGAAAGCAACGGCCAGCCTGAGAACAACTACAAAACCACCCCCCCAGTGCTG | |
| | TGGGAATCCAATGGCCAGCCCGAGAACAACTAC | GACTCCGACGGATCTTTCTTCCTGTACTCTAAGTTGACCGTGGACAAGTCCAGGTGGCAGCA | |
| | AAAACCACCCCCCCCGTGCTGGACTCCGATGGA | GGGAAACGTGTTCTCCTGCTCTGTGATGCACGAGGCCCTGCACAACCACTATACCCAGAAGA | |
| | TCTTTCTTCCTGGTGTCCAAGCTGACCGTGGAC | GTTTGTCCCTGTCCCCCGGCAAGGCTGCTGAACCTGTGGAAGATAATGTGATCAATTTTGTG | |
| | AAGTCCAGATGGCAGCAGGGCAATGTCTTTTCC | GCCATGAAATTTATTGACAACACCCTGTATTTCATCGCCGAAGATGATGAAAACATTGAACA | |
| | TGCTCCGTGATGCACGAGGCCCTCCATAACAGA | GGACTATTTCGGAAAGCTGGAATCCAAATTGAGCGTGATCCGGAACCTGAACGATCAGGTGC | |
| | TTCACCCAGAAGTCCCTGTCCCTGTCTCCCGGA | TGTTCATCGACCAGGGCAACAGGCCTCTGTTCGAGGACATGACCGACTCCGACGTGAGGGAC | |
| | AAG (SEQ ID NO: 125) | AACGCCCCTAGAACAATCTTCATCATCTCCAAGTACGGAGACTCCCAGCCAAGGGGCCTGGC | |
| | | TGTTACAATCTCCGTGAAGGTGGAGAAGATCTCCACCCTGTCCGTGGAGAACAAGATCATCT | |
| | | CCTTCAAGGAGATGAACCCCCCCGACAACATCAAGGACACCAAATCCGACATCATCTTCTTC | |
| | | CAGCGCTCCGTGCCTGGGCACGATAACAAAGTCCAGTTCGAGTCCAGCTCCTACGAGGGCTA | |
| | | CTTCCTGGCCGTCGAAAAGGAAAGGGACCTGTTCAAGCTGATCCTGAAGAAGGAGGACGAGC | |
| | | TGGGAGACAGGAGCATCATGTTCACCGTCCAGAACGAGGAC (SEQ ID NO: 166) | |
| FUSE-645 | (SEQ ID NO: 114) | GAAGTGCAGCTGGTGGAGAGCGGAGGAGGATTGGTGCAGGCAGGAGGAAGCCTGAGGCTGAG | |
| | | TTGTGCCGCTAGTGGAAGGACATTTAGTTCCTATGCAATGGGGTGGTTCAGGCAGGCCCCAG | |
| | | GAAAGGAAAGGGAGTTTGTGGTGGCCATCAACTGGAGTAGCGGATCTACATACTACGCTGAT | |
| | | TCTGTGAAGGGCAGGTTCACCATCAGCCGGGACAATGCTAAGAACACCATGTACCTGCAGAT | |
| | | GAACAGCCTGAAGCCTGAGGACACCGCCGTGTACTATTGCGCCGCTGGATATCAGATCAATT | |
| | | CCGGCAACTACAACTTCAAGGACTACGAGTATGACTACTGGGGCCAGGGCACACAGGTGACA | |
| | | GTGTCATCCGAACCTAAATCCTCCGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAAGCTGCTGAACCTGTGGAAGATAATTCTATTAATTTCGTG | |
| | | GCCATGAAATTCATCGATAATACTCTGTACTTCATCGCCGAAGATGATGAAAATATTGAACA | |
| | | GGACTATTTCGGAAAGCTGGAAAGCAAACTGAGCGTGATCAGGAACCTGAACGATCAGGTGC | |
| | | TGTTCATCGACCAGGGCAACCGCCCTCTGTTCGAGGATATGACCGACTCCGACAGCAGGGAC | |
| | | AACGCACCAAGGACCATCTTCATCATCTCCAAGTACGGCGACTCCCAGCCAAGGGGACTGGC | |
| | | TGTTACCATCTCCGTGAAGTCCGAGAAGATTTCCACCCTGAGCTCCGAGAACAAGATCATTT | |
| | | CCTTCAAGGAGATGAACCCCCCCGACAACATCAAAGACACCAAATCCGACATCATCTTCTTC | |
| | | CAGCGCAGCGTGCCCGGACACGATAACAAAGTGCAGTTTGAGTCCAGCTCCTACGAGGGCTA | |
| | | CTTCCTGGCCTCCGAAAAGGAAAGGGACCTGTTCAAGCTGATCCTGAAGAAGGAGGACGAGT | |
| | | TGGGGGACAGGTCAATTATGTTCACCGTTCAGAACGAGGAC (SEQ ID NO: 167) | |
| FUSE-658 | GAGCCCAAATCTTCTGACAAAACTCACACATGC | (SEQ ID NO: 154) | |
| | CCACCGTGCCCAGCACCTGAACTCCTGGGGGGA | | |
| | CCGTCAGTCTTCCTCTTCCCCCCAAAACCCAAG | | |
| | GACACCCTCATGATCTCCCGGACCCCCGAGGTC | | |
| | ACATGCGTGGTGGTGGACGTGAGCCACGAAGAC | | |
| | CCTGAGGTCAAGTTCAACTGGTACGTGGACGGC | | |
| | GTGGAGGTGCATAATGCCAAGACAAAGCCGCGG | | |
| | GAGGAGCAGTACAACAGCACGTACCGTGTGGTC | | |
| | AGCGTCCTCACCGTCCTGCACCAGGACTGGCTG | | |
| | AATGGCAAGGAGTACAAGTGCAAGGTCTCCAAC | | |
| | AAAGCCCTCCCAGCCCCCATCGAGAAAACCATC | | |
| | TCCAAAGCCAAAGGGCAGCCCCGAGAACCACAG | | |
| | GTGTACACCCTGCCCCCATCCCGGGAGGAGATG | | |
| | ACCAAGAACCAGGTCAGCCTGAGCTGCGCCGTC | | |
| | AAAGGCTTCTATCCCAGCGACATCGCCGTGGAG | | |
| | TGGGAGAGCAATGGGCAGCCGGAGAACAACTAC | | |
| | AAGACCACGCCTCCCGTGCTGGACTCCGACGGC | | |
| | TCCTTCTTCCTCGTGAGCAAGCTCACCGTGGAC | | |
| | AAGAGCAGGTGGCAGCAGGGGAACGTCTTCTCA | | |
| | TGCTCCGTGATGCATGAGGCTCTGCACAACCGG | | |
| | TTCACGCAGAAGAGCCTCTCCCTGTCTCCGGGT | | |
| | AAAGCCGCCGAGCCCGTCGAGGACAACGTGATT | | |
| | AATTTCGTGGCTATGAAGTTCATCGATAACACA | | |
| | CTCTATTTTATCGCTGAGGACGATGAGAATATT | | |
| | GGCAACAGGTCCTATTTCGGCAAGTTGGAAAGC | | |
| | AAGCTGTCAGTGATCCGCAATCTCAATGATCAG | | |
| | GTGCTGTTTATTGATCAGGGCAATAGGCCACTC | | |
| | TTTGAAGATATGACCGATTCTGATGTCCGCGAC | | |
| | AATGCACCTAGAACTATCTTTATTATTTCCAAG | | |
| | TATGGGGACAGTCAGCCTCGGGGACTGGCAGTC | | |
| | ACTATTTCCGTCAAGGTGGAGAAGATTAGCACT | | |
| | CTCAGCGTTGAGAACAAAATTATCTCTTTCAAG | | |
| | GAGATGAACCCACCAGATAACATTAAGGACACA | | |
| | AAGTCCGATATCATCTTCTTCCAGCGAAGCGTT | | |
| | CCAGGCCATGATAATAAGGTGCAGTTCGAGTCT | | |
| | AGCAGCTATGAGGGCTATTTTCTGGCCGTTGAG | | |
| | AAGGAGCGAGATTTGTTTAAGCTCATCCTGAAG | | |
| | AAGGAGGATGAGTTGGGGGATAGAAGTATCATG | | |
| | TTTACTGTCCAGAACGAGGAT (SEQ ID | | |
| | NO: 126) | | |
| FUSE-659 | GAGCCCAAATCTTCTGACAAAACTCACACATGC | (SEQ ID NO: 154) | |
| | CCACCGTGCCCAGCACCTGAACTCCTGGGGGGA | | |
| | CCGTCAGTCTTCCTCTTCCCCCCAAAACCCAAG | | |
| | GACACCCTCATGATCTCCCGGACCCCCGAGGTC | | |
| | ACATGCGTGGTGGTGGACGTGAGCCACGAAGAC | | |
| | CCTGAGGTCAAGTTCAACTGGTACGTGGACGGC | | |
| | GTGGAGGTGCATAATGCCAAGACAAAGCCGCGG | | |
| | GAGGAGCAGTACAACAGCACGTACCGTGTGGTC | | |
| | AGCGTCCTCACCGTCCTGCACCAGGACTGGCTG | | |
| | AATGGCAAGGAGTACAAGTGCAAGGTCTCCAAC | | |
| | AAAGCCCTCCCAGCCCCCATCGAGAAAACCATC | | |
| | TCCAAAGCCAAAGGGCAGCCCCGAGAACCACAG | | |
| | GTGTACACCCTGCCCCCATCCCGGGAGGAGATG | | |
| | ACCAAGAACCAGGTCAGCCTGAGCTGCGCCGTC | | |
| | AAAGGCTTCTATCCCAGCGACATCGCCGTGGAG | | |
| | TGGGAGAGCAATGGGCAGCCGGAGAACAACTAC | | |
| | AAGACCACGCCTCCCGTGCTGGACTCCGACGGC | | |
| | TCCTTCTTCCTCGTGAGCAAGCTCACCGTGGAC | | |
| | AAGAGCAGGTGGCAGCAGGGGAACGTCTTCTCA | | |
| | TGCTCCGTGATGCATGAGGCTCTGCACAACCGG | | |
| | TTCACGCAGAAGAGCCTCTCCCTGTCTCCGGGT | | |
| | AAAGCTGCCGAACCCGTGGAGGACAATGTGATC | | |
| | AATTTTGTGGCTATGAAGTTTATTGATAACACC | | |
| | CTCTACTTTATCGCCGAAGATGACGAAAATGGA | | |
| | GGCGGCGGCAAGCCTCTGGGCCTGCAGGCTAGA | | |
| | GTCGTTGGCGGAGGAGGTACATACTTTGGCAAA | | |
| | CTGGAGTCCAAGCTCTCTGTGATTCGGAACCTG | | |
| | AATGATCAGGTCTTGTTCATCGATCAGGGAAAT | | |
| | CGGCCTTTGTTTGAAGATATGACCGACTCCGAC | | |
| | GTGCGAGATAACGCCCCAAGGACCATTTTCATC | | |
| | ATCTCCAAATATGGTGACTCCCAGCCTCGGGGA | | |
| | CTCGCAGTCACCATTTCAGTGAAGGTGGAGAAG | | |
| | ATTTCTACCTTGTCTGTTGAGAATAAAATTATC | | |
| | TCATTCAAAGAGATGAATCCTCCAGATAATATT | | |
| | AAAGACACAAAATCAGACATCATTTTCTTCCAG | | |
| | CGGTCTGTGCCTGGCCACGACAATAAAGTGCAG | | |
| | TTCGAATCCTCTTCTTATGAGGGGTACTTCCTG | | |
| | GCTGTCGAGAAGGAAAGGGACCTGTTTAAGCTG | | |
| | ATTCTGAAGAAAGAGGATGAGTTGGGTGACCGA | | |
| | AGCATTATGTTTACCGTCCAGAATGAAGAT | | |
| | (SEQ ID NO: 127) | | |
| FUSE-660 | GAGCCCAAATCTTCTGACAAAACTCACACATGC | (SEQ ID NO: 154) | |
| | CCACCGTGCCCAGCACCTGAACTCCTGGGGGGA | | |
| | CCGTCAGTCTTCCTCTTCCCCCCAAAACCCAAG | | |
| | GACACCCTCATGATCTCCCGGACCCCCGAGGTC | | |
| | ACATGCGTGGTGGTGGACGTGAGCCACGAAGAC | | |
| | CCTGAGGTCAAGTTCAACTGGTACGTGGACGGC | | |
| | GTGGAGGTGCATAATGCCAAGACAAAGCCGCGG | | |
| | GAGGAGCAGTACAACAGCACGTACCGTGTGGTC | | |
| | AGCGTCCTCACCGTCCTGCACCAGGACTGGCTG | | |
| | AATGGCAAGGAGTACAAGTGCAAGGTCTCCAAC | | |
| | AAAGCCCTCCCAGCCCCCATCGAGAAAACCATC | | |
| | TCCAAAGCCAAAGGGCAGCCCCGAGAACCACAG | | |
| | GTGTACACCCTGCCCCCATCCCGGGAGGAGATG | | |
| | ACCAAGAACCAGGTCAGCCTGAGCTGCGCCGTC | | |
| | AAAGGCTTCTATCCCAGCGACATCGCCGTGGAG | | |
| | TGGGAGAGCAATGGGCAGCCGGAGAACAACTAC | | |
| | AAGACCACGCCTCCCGTGCTGGACTCCGACGGC | | |
| | TCCTTCTTCCTCGTGAGCAAGCTCACCGTGGAC | | |
| | AAGAGCAGGTGGCAGCAGGGGAACGTCTTCTCA | | |
| | TGCTCCGTGATGCATGAGGCTCTGCACAACCGG | | |
| | TTCACGCAGAAGAGCCTCTCCCTGTCTCCGGGT | | |
| | AAAGCCGCCGAGCCCGTCGAAGATAACGTGATC | | |
| | AACTTCGTGGCCATGAAATTCATCGATAATACA | | |
| | CTCTACTTTATTGCCGAGGATGACGAGAACATC | | |
| | GAGCAGGATGGCGGCTCCATCGGCAACAGAAGT | | |
| | GGCGGCGGAGGCAAGCCCTTGGGACTGCAGGCC | | |
| | AGGGTTGTCGGAGGTGGGGGTACTATTGAGCAG | | |
| | GATTACTTTGGGAAGTTGGAATCCAAGCTCAGT | | |
| | GTGATCCGCAATCTGAATGATCAGGTGCTCTTC | | |
| | ATCGACCAGGGGAATCGGCCCCTGTTCGAAGAT | | |
| | ATGACCGACTCCGATGTTAGGGATAACGCTCCT | | |
| | AGAACTATCTTTATCATTAGCAAGTATGGGGAC | | |
| | TCTCAGCCAAGAGGGCTGGCCGTGACCATCTCC | | |
| | GTCAAAGTCGAAAAGATTTCCACTCTCTCTGTG | | |
| | GAAAATAAGATCATCAGCTTTAAAGAGATGAAT | | |
| | CCCCCTGACAACATCAAGGATACAAAATCTGAT | | |
| | ATCATCTTTTTCCAGCGCTCTGTTCCCGGACAT | | |
| | GACAACAAGGTGCAGTTTGAGTCTAGCAGCTAC | | |
| | GAGGGGTATTTCCTCGCTGTTGAAAAGGAACGA | | |
| | GATCTCTTTAAATTGATTTTGAAGAAGGAAGAT | | |
| | GAGCTGGGAGACAGGTCAATCATGTTCACCGTT | | |
| | CAGAACGAAGAT (SEQ ID NO: 128) | | |
| FUSE-669 | (SEQ ID NO: 114) | SEQ ID NO: 288 | SEQ |
| | | | ID |
| | | | NO: 186 |
| FUSE-670 | CAGGTTCAGCTCAAACAGAGCGGCCCCGGACTC | — | SEQ |
| | GTTCAGCCATCTCAGTCTCTCTCCATCACATGC | | ID |
| | ACAGTCTCCGGCTTCTCCCTCACAAACTACGGC | | NO: 186 |
| | GTCCACTGGGTCAGGCAGTCCCCTGGAAAGGGA | | |
| | CTCGAATGGCTGGGAGTGATTTGGTCCGGCGGC | | |
| | AACACCGACTACAACACCCCTTTCACCTCTCGG | | |
| | CTGTCCATCAACAAGGACAATTCCAAGTCCCAG | | |
| | GTCTTTTTCAAGATGAATAGCCTGCAGTCCAAC | | |
| | GACACCGCCATCTACTACTGCGCCCGGGCTTTG | | |
| | ACATACTACGACTACGAGTTCGCCTACTGGGGC | | |
| | CAGGGAACCTTGGTGACAGTGTCCGCTGCTAGC | | |
| | ACCAAGGGCCCATCGGTCTTCCCCCTGGCACCC | | |
| | TCCTCCAAGAGCACCTCTGGGGGCACAGCGGCC | | |
| | CTGGGCTGCCTGGTCAAGGACTACTTCCCCGAA | | |
| | CCGGTGACGGTGTCGTGGAACTCAGGCGCCCTG | | |
| | ACCAGCGGCGTGCACACCTTCCCGGCTGTCCTA | | |
| | CAGTCCTCAGGACTCTACTCCCTCAGCAGCGTG | | |
| | GTGACCGTGCCCTCCAGCAGCTTGGGCACCCAG | | |
| | ACCTACATCTGCAACGTGAATCACAAGCCCAGC | | |
| | AACACCAAGGTGGACAAGAAAGTTGAGCCCAAA | | |
| | TCTTGTGACAAAACTCACACATGCCCACCGTGC | | |
| | CCAGCACCTGAACTCCTGGGGGGACCGTCAGTC | | |
| | TTCCTCTTCCCCCCAAAACCCAAGGACACCCTC | | |
| | ATGATCTCCCGGACCCCCGAGGTCACATGCGTG | | |
| | GTGGTGGACGTGAGCCACGAAGACCCTGAGGTC | | |
| | AAGTTCAACTGGTACGTGGACGGCGTGGAGGTG | | |
| | CATAATGCCAAGACAAAGCCGCGGGAGGAGCAG | | |
| | TACAACAGCACGTACCGTGTGGTCAGCGTCCTC | | |
| | ACCGTCCTGCACCAGGACTGGCTGAATGGCAAG | | |
| | GAGTACAAGTGCAAGGTCTCCAACAAAGCCCTC | | |
| | CCAGCCCCCATCGAGAAAACCATCTCCAAAGCC | | |
| | AAAGGGCAGCCCCGAGAACCACAGGTGTACACC | | |
| | CTGCCCCCATCCCGGGACGAGCTGACCAAGAAC | | |
| | CAGGTCAGCCTGACCTGCCTGGTCAAAGGCTTC | | |
| | TATCCCAGCGACATCGCCGTGGAGTGGGAGAGC | | |
| | AATGGGCAGCCGGAGAACAACTACAAGACCACG | | |
| | CCTCCCGTGCTGGACTCCGACGGCTCCTTCTTC | | |
| | CTCTACAGCAAGCTCACCGTGGACAAGAGCAGG | | |
| | TGGCAGCAGGGGAACGTCTTCTCATGCTCCGTG | | |
| | ATGCATGAGGCTCTGCACAACCACTACACGCAG | | |
| | AAGAGCCTCTCCCTGTCTCCGGGTAAAGGAGGC | | |
| | GGGGGATCTGGGGGAGGAGGTAGTGGAGGCGGA | | |
| | GGATCCGGCGGTGGAGGTTCTGGTGGTGGGGGC | | |
| | AGTGGGGGTGGCGGATCAGCTGCTGAACCTGTG | | |
| | GAAGATAATGTGATCAATTTTGTGGCCATGAAA | | |
| | TTTATTGACAACACCCTGTATTTCATCGCCGAA | | |
| | GATGATGAAAACATTGAACAGGACTATTTCGGA | | |
| | AAGCTGGAATCCAAATTGAGCGTGATCCGGAAC | | |
| | CTGAACGATCAGGTGCTGTTCATCGACCAGGGC | | |
| | AACAGGCCTCTGTTCGAGGACATGACCGACTCC | | |
| | GACGTGAGGGACAACGCCCCTAGAACAATCTTC | | |
| | ATCATCTCCAAGTACGGAGACTCCCAGCCAAGG | | |
| | GGCCTGGCTGTTACAATCTCCGTGAAGGTGGAG | | |
| | AAGATCTCCACCCTGTCCGTGGAGAACAAGATC | | |
| | ATCTCCTTCAAGGAGATGAACCCCCCCGACAAC | | |
| | ATCAAGGACACCAAATCCGACATCATCTTCTTC | | |
| | CAGCGCTCCGTGCCTGGGCACGATAACAAAGTC | | |
| | CAGTTCGAGTCCAGCTCCTACGAGGGCTACTTC | | |
| | CTGGCCGTCGAAAAGGAAAGGGACCTGTTCAAG | | |
| | CTGATCCTGAAGAAGGAGGACGAGCTGGGAGAC | | |
| | AGGAGCATCATGTTCACCGTCCAGAACGAGGAC | | |
| | (SEQ ID NO: 290) | | |
| FUSE-675 | GAAGTGCAGCTGGTGGAGAGCGGAGGAGGATTG | — | — |
| | GTGCAGGCAGGAGGAAGCCTGAGGCTGAGTTGT | | |
| | GCCGCTAGTGGAAGGACATTTAGTTCCTATGCA | | |
| | ATGGGGTGGTTCAGGCAGGCCCCAGGAAAGGAA | | |
| | AGGGAGTTTGTGGTGGCCATCAACTGGAGTAGC | | |
| | GGATCTACATACTACGCTGATTCTGTGAAGGGC | | |
| | AGGTTCACCATCAGCCGGGACAATGCTAAGAAC | | |
| | ACCATGTACCTGCAGATGTCCTCCCTGAAGCCT | | |
| | GAAGATACTGCTGTGTACTACTGCGCCGCCGGC | | |
| | TATCAGATCAATTCCGGAAACTACAACTTCAAA | | |
| | GATTACGAGTATGACTACTGGGGCCAGGGTACC | | |
| | CAGGTGACAGTGAGTTCCGAGCCCAAATCTGCT | | |
| | GACAAAACTCACACATGCCCACCGTGCCCAGCA | | |
| | CCTGAACTCCTGGGGGGACCGTCAGTCTTCCTC | | |
| | TTCCCCCCAAAACCCAAGGACACCCTCATGATC | | |
| | TCCCGGACCCCCGAGGTCACATGCGTGGTGGTG | | |
| | GACGTGAGCCACGAAGACCCTGAGGTCAAGTTC | | |
| | AACTGGTACGTGGACGGCGTGGAGGTGCATAAT | | |
| | GCCAAGACAAAGCCGCGGGAGGAGCAGTACAAC | | |
| | AGCACGTACCGTGTGGTCAGCGTCCTCACCGTC | | |
| | CTGCACCAGGACTGGCTGAATGGCAAGGAGTAC | | |
| | AAGTGCAAGGTCTCCAACAAAGCCCTCCCAGCC | | |
| | CCCATCGAGAAAACCATCTCCAAAGCCAAAGGG | | |
| | CAGCCCCGAGAACCACAGGTGTACACCCTGCCC | | |
| | CCATCCCGGGAGGAGATGACCAAGAACCAGGTC | | |
| | AGCCTGACCTGCCTGGTCAAAGGCTTCTATCCC | | |
| | AGCGACATCGCCGTGGAGTGGGAGAGCAATGGG | | |
| | CAGCCGGAGAACAACTACAAGACCACGCCTCCC | | |
| | GTGCTGGACTCCGACGGCTCCTTCTTCCTCTAC | | |
| | AGCAAGCTCACCGTGGACAAGAGCAGGTGGCAG | | |
| | CAGGGGAACGTCTTCTCATGCTCCGTGATGCAT | | |
| | GAGGCTCTGCACAACCACTACACGCAGAAGAGC | | |
| | CTCTCCCTGTCTCCGGGTAAA (SEQ ID | | |
| | NO: 291) | | |
| FUSE-676 | GAAGTGCAGCTGGTGGAGAGCGGAGGAGGATTG | — | — |
| | GTGCAGGCAGGAGGAAGCCTGAGGCTGAGTTGT | | |
| | GCCGCTAGTGGAAGGACATTTAGTTCCTATGCA | | |
| | ATGGGGTGGTTCAGGCAGGCCCCAGGAAAGGAA | | |
| | AGGGAGTTTGTGGTGGCCATCAACTGGAGTAGC | | |
| | GGATCTACATACTACGCTGATTCTGTGAAGGGC | | |
| | AGGTTCACCATCAGCCGGGACAATGCTAAGAAC | | |
| | ACCATGTACCTGCAGATGTCCTCCCTGAAGCCT | | |
| | GAAGATACTGCTGTGTACTACTGCGCCGCCGGC | | |
| | TATCAGATCAATTCCGGAAACTACAACTTCAAA | | |
| | GATTACGAGTATGACTACTGGGGCCAGGGTACC | | |
| | CAGGTGACAGTGAGTTCCGAGCCCAAATCTGCT | | |
| | GACAAAACTCACACATGCCCACCGTGCCCAGCA | | |
| | CCTGAACTCCTGGGGGGACCGTCAGTCTTCCTC | | |
| | TTCCCCCCAAAACCCAAGGACACCCTCATGATC | | |
| | TCCCGGACCCCCGAGGTCACATGCGTGGTGGTG | | |
| | GACGTGAGCCACGAAGACCCTGAGGTCAAGTTC | | |
| | AACTGGTACGTGGACGGCGTGGAGGTGCATAAT | | |
| | GCCAAGACAAAGCCGCGGGAGGAGCAGTACAAC | | |
| | AGCACGTACCGTGTGGTCAGCGTCCTCACCGTC | | |
| | CTGCACCAGGACTGGCTGAATGGCAAGGAGTAC | | |
| | AAGTGCAAGGTCTCCAACAAAGCCCTCCCAGCC | | |
| | CCCATCGAGAAAACCATCTCCAAAGCCAAAGGG | | |
| | CAGCCCCGAGAACCACAGGTGTACACCCTGCCC | | |
| | CCATCCCGGGAGGAGATGACCAAGAACCAGGTC | | |
| | AGCCTGACCTGCCTGGTCAAAGGCTTCTATCCC | | |
| | AGCGACATCGCCGTGGAGTGGGAGAGCAATGGG | | |
| | CAGCCGGAGAACAACTACAAGACCACGCCTCCC | | |
| | GTGCTGGACTCCGACGGCTCCTTCTTCCTCTAC | | |
| | AGCAAGCTCACCGTGGACAAGAGCAGGTGGCAG | | |
| | CAGGGGAACGTCTTCTCATGCTCCGTGATGCAT | | |
| | GAGGCTCTGCACAACCACTACACGCAGAAGAGC | | |
| | CTCTCCCTGTCTCCGGGTAAAGGAGGCGGGGGA | | |
| | TCTGGGGGAGGAGGTAGTGGAGGCGGAGGATCC | | |
| | GGCGGTGGAGGTTCTGGTGGTGGGGGCAGTGGG | | |
| | GGTGGCGGATCAGCTGCTGAACCTGTGGAAGAT | | |
| | AATGTGATCAATTTTGTGGCCATGAAATTTATT | | |
| | GACAACACCCTGTATTTCATCGCCGAAGATGAT | | |
| | GAAAACATTGAACAGGACTATTTCGGAAAGCTG | | |
| | GAATCCAAATTGAGCGTGATCCGGAACCTGAAC | | |
| | GATCAGGTGCTGTTCATCGACCAGGGCAACAGG | | |
| | CCTCTGTTCGAGGACATGACCGACTCCGACGTG | | |
| | AGGGACAACGCCCCTAGAACAATCTTCATCATC | | |
| | TCCAAGTACGGAGACTCCCAGCCAAGGGGCCTG | | |
| | GCTGTTACAATCTCCGTGAAGGTGGAGAAGATC | | |
| | TCCACCCTGTCCGTGGAGAACAAGATCATCTCC | | |
| | TTCAAGGAGATGAACCCCCCCGACAACATCAAG | | |
| | GACACCAAATCCGACATCATCTTCTTCCAGCGC | | |
| | TCCGTGCCTGGGCACGATAACAAAGTCCAGTTC | | |
| | GAGTCCAGCTCCTACGAGGGCTACTTCCTGGCC | | |
| | GTCGAAAAGGAAAGGGACCTGTTCAAGCTGATC | | |
| | CTGAAGAAGGAGGACGAGCTGGGAGACAGGAGC | | |
| | ATCATGTTCACCGTCCAGAACGAGGAC (SEQ | | |
| | ID NO: 292) | | |
| FUSE-682 | GAAGTGCAGCTGGTGGAGAGCGGCGGCGGACTG | (SEQ ID NO: 148) | — |
| | GTCCAGGCTGGAGGCAGCCTGCGGCTGTCCTGT | | |
| | GCCGCTTCTGGCAGGACCTTCTCCAGCTATGCC | | |
| | ATGGGATGGTTTCGGCAGGCCCCTGGAAAAGAG | | |
| | CGCGAGTTCGTGGTGGCCATCAACTGGAGTAGC | | |
| | GGTTCTACATATTACGCAGATAGCGTCAAGGGT | | |
| | CGTTTTACTATTTCCCGAGATAACGCCAAGAAC | | |
| | ACCATGTACCTGCAGATGTCATCTCTGAAGCCA | | |
| | GAGGATACTGCTGTGTACTATTGTGCCGCCGGC | | |
| | TATCAGATCAACTCCGGCAACTACAACTTTAAG | | |
| | GATTACGAGTACGATTACTGGGGACAGGGTACA | | |
| | CAGGTCACCGTGTCAAGTGAGCCCAAATCTGCT | | |
| | GACAAAACTCACACATGCCCACCGTGCCCAGCA | | |
| | CCTGAACTCCTGGGGGGACCGTCAGTCTTCCTC | | |
| | TTCCCCCCAAAACCCAAGGACACCCTCATGATC | | |
| | TCCCGGACCCCCGAGGTCACATGCGTGGTGGTG | | |
| | GACGTGAGCCACGAAGACCCTGAGGTCAAGTTC | | |
| | AACTGGTACGTGGACGGCGTGGAGGTGCATAAT | | |
| | GCCAAGACAAAGCCGCGGGAGGAGCAGTACAAC | | |
| | AGCACGTACCGTGTGGTCAGCGTCCTCACCGTC | | |
| | CTGCACCAGGACTGGCTGAATGGCAAGGAGTAC | | |
| | AAGTGCAAGGTCTCCAACAAAGCCCTCCCAGCC | | |
| | CCCATCGAGAAAACCATCTCCAAAGCCAAAGGG | | |
| | CAGCCCCGAGAACCACAGGTGTACACCCTGCCC | | |
| | CCATCCCGGGAGGAGATGACCAAGAACCAGGTC | | |
| | AGCCTGAGCTGCGCCGTCAAAGGCTTCTATCCC | | |
| | AGCGACATCGCCGTGGAGTGGGAGAGCAATGGG | | |
| | CAGCCGGAGAACAACTACAAGACCACGCCTCCC | | |
| | GTGCTGGACTCCGACGGCTCCTTCTTCCTCGTG | | |
| | AGCAAGCTCACCGTGGACAAGAGCAGGTGGCAG | | |
| | CAGGGGAACGTCTTCTCATGCTCCGTGATGCAT | | |
| | GAGGCTCTGCACAACCGGTTCACGCAGAAGAGC | | |
| | CTCTCCCTGTCTCCGGGTAAA (SEQ ID | | |
| | NO: 293) | | |
| FUSE-683 | GAGCCCAAATCTGCTGACAAAACTCACACATGC | GAAGTGCAGCTGGTGGAGAGCGGCGGCGGACTGGTCCAGGCTGGAGGCAGCCTGCGGCTGTC | — |
| | CCACCGTGCCCAGCACCTGAACTCCTGGGGGGA | CTGTGCCGCTTCTGGCAGGACCTTCTCCAGCTATGCCATGGGATGGTTTCGGCAGGCCCCTG | |
| | CCGTCAGTCTTCCTCTTCCCCCCAAAACCCAAG | GAAAAGAGCGCGAGTTCGTGGTGGCCATCAACTGGAGTAGCGGTTCTACATATTACGCAGAT | |
| | GACACCCTCATGATCTCCCGGACCCCCGAGGTC | AGCGTCAAGGGTCGTTTTACTATTTCCCGAGATAACGCCAAGAACACCATGTACCTGCAGAT | |
| | ACATGCGTGGTGGTGGACGTGAGCCACGAAGAC | GTCATCTCTGAAGCCAGAGGATACTGCTGTGTACTATTGTGCCGCCGGCTATCAGATCAACT | |
| | CCTGAGGTCAAGTTCAACTGGTACGTGGACGGC | CCGGCAACTACAACTTTAAGGATTACGAGTACGATTACTGGGGACAGGGTACACAGGTCACC | |
| | GTGGAGGTGCATAATGCCAAGACAAAGCCGCGG | GTGTCAAGTGAGCCCAAATCTTCTGACAAAACTCACACATGCCCACCGTGCCCAGCACCTGA | |
| | GAGGAGCAGTACAACAGCACGTACCGTGTGGTC | ACTCCTGGGGGGACCGTCAGTCTTCCTCTTCCCCCCAAAACCCAAGGACACCCTCATGATCT | |
| | AGCGTCCTCACCGTCCTGCACCAGGACTGGCTG | CCCGGACCCCCGAGGTCACATGCGTGGTGGTGGACGTGAGCCACGAAGACCCTGAGGTCAAG | |
| | AATGGCAAGGAGTACAAGTGCAAGGTCTCCAAC | TTCAACTGGTACGTGGACGGCGTGGAGGTGCATAATGCCAAGACAAAGCCGCGGGAGGAGCA | |
| | AAAGCCCTCCCAGCCCCCATCGAGAAAACCATC | GTACAACAGCACGTACCGTGTGGTCAGCGTCCTCACCGTCCTGCACCAGGACTGGCTGAATG | |
| | TCCAAAGCCAAAGGGCAGCCCCGAGAACCACAG | GCAAGGAGTACAAGTGCAAGGTCTCCAACAAAGCCCTCCCAGCCCCCATCGAGAAAACCATC | |
| | GTGTACACCCTGCCCCCATCCCGGGAGGAGATG | TCCAAAGCCAAAGGGCAGCCCCGAGAACCACAGGTGTACACCCTGCCCCCATCCCGGGAGGA | |
| | ACCAAGAACCAGGTCAGCCTGAGCTGCGCCGTC | GATGACCAAGAACCAGGTCAGCCTGTGGTGCCTGGTCAAAGGCTTCTATCCCAGCGACATCG | |
| | AAAGGCTTCTATCCCAGCGACATCGCCGTGGAG | CCGTGGAGTGGGAGAGCAATGGGCAGCCGGAGAACAACTACAAGACCACGCCTCCCGTGCTG | |
| | TGGGAGAGCAATGGGCAGCCGGAGAACAACTAC | GACTCCGACGGCTCCTTCTTCCTCTACAGCAAGCTCACCGTGGACAAGAGCAGGTGGCAGCA | |
| | AAGACCACGCCTCCCGTGCTGGACTCCGACGGC | GGGGAACGTCTTCTCATGCTCCGTGATGCATGAGGCTCTGCACAACCACTACACGCAGAAGA | |
| | TCCTTCTTCCTCGTGAGCAAGCTCACCGTGGAC | GCCTCTCCCTGTCTCCGGGTAAAGGAGGCGGGGGATCTGGGGGAGGAGGTAGTGGAGGCGGA | |
| | AAGAGCAGGTGGCAGCAGGGGAACGTCTTCTCA | GGATCCGGCGGTGGAGGTTCTGGTGGTGGGGGCAGTGGGGGTGGCGGATCAGCTGCTGAACC | |
| | TGCTCCGTGATGCATGAGGCTCTGCACAACCGG | TGTGGAAGATAATGTGATCAATTTCGTGGCCATGAAGTTCATTGACAACACCCTGTACTTCA | |
| | TTCACGCAGAAGAGCCTCTCCCTGTCTCCGGGT | TCGCTGAAGATGATGAAAACATCGAGCAGGACTATTTCGGAAAACTGGAAAGCAAACTGAGC | |
| | AAAGGAGGCGGGGGATCTGGGGGAGGAGGTAGT | GTGATCAGGAACCTGAACGACCAGGTGCTGTTTATCGACCAGGGCAACAGGCCCCTGTTCGA | |
| | GGAGGCGGAGGATCCGGCGGTGGAGGTTCTGGT | AGATATGACCGACAGCGACGTCAGGGACAACGCTCCTAGAACAATCTTCATCATCTCCAAGT | |
| | GGTGGGGGCAGTGGGGGTGGCGGATCAGCTGCT | ACGGCGACTCCCAGCCAAGGGGACTGGCTGTGACCATTTCCGTGAAAGTGGAGAAGATCTCC | |
| | GAACCTGTGGAAGATAATGTGATCAATTTCGTG | ACACTGAGCGTGGAAAACAAAATCATCTCCTTCAAGGAGATGAACCCCCCCGACAACATTAA | |
| | GCCATGAAGTTCATTGACAACACCCTGTACTTC | AGACACCAAAAGCGATATCATCTTCTTCCAGAGGAGTGTGCCCGGCCACGATAACAAAGTGC | |
| | ATCGCTGAAGATGATGAAAACATCGAGCAGGAC | AGTTCGAGAGCAGCTCCTACGAGGGATACTTTCTGGCCGTCGAAAAGGAGAGGGACCTGTTC | |
| | TATTTCGGAAAACTGGAAAGCAAACTGAGCGTG | AAGCTGATCCTGAAGAAGGAGGACGAGCTGGGGGACAGATCAATCATGTTCACCGTGCAGAA | |
| | ATCAGGAACCTGAACGACCAGGTGCTGTTTATC | CGAAGAC (SEQ ID NO: 307) | |
| | GACCAGGGCAACAGGCCCCTGTTCGAAGATATG | | |
| | ACCGACAGCGACGTCAGGGACAACGCTCCTAGA | | |
| | ACAATCTTCATCATCTCCAAGTACGGCGACTCC | | |
| | CAGCCAAGGGGACTGGCTGTGACCATTTCCGTG | | |
| | AAAGTGGAGAAGATCTCCACACTGAGCGTGGAA | | |
| | AACAAAATCATCTCCTTCAAGGAGATGAACCCC | | |
| | CCCGACAACATTAAAGACACCAAAAGCGATATC | | |
| | ATCTTCTTCCAGAGGAGTGTGCCCGGCCACGAT | | |
| | AACAAAGTGCAGTTCGAGAGCAGCTCCTACGAG | | |
| | GGATACTTTCTGGCCGTCGAAAAGGAGAGGGAC | | |
| | CTGTTCAAGCTGATCCTGAAGAAGGAGGACGAG | | |
| | CTGGGGGACAGATCAATCATGTTCACCGTGCAG | | |
| | AACGAAGAC (SEQ ID NO: 294) | | |
| FUSE-684 | (SEQ ID NO: 294) | CAGGTGCAGCTGAAGCAGTCTGGACCAGGACTCGTGCAGCCAAGTCAGTCTCTCTCCATCAC | SEQ |
| | | ATGCACAGTCTCCGGCTTCTCCCTCACAAACTACGGCGTCCACTGGGTGCGACAGTCACCTG | ID |
| | | GAAAAGGCCTCGAATGGCTCGGCGTGATCTGGTCCGGAGGAAATACCGACTACAACACCCCC | NO: 186 |
| | | TTTACCTCCAGGCTGTCCATCAACAAGGATAATTCCAAGTCCCAGGTGTTCTTCAAGATGAA | |
| | | TAGCCTGCAGTCCAACGATACCGCCATCTACTACTGCGCCAGGGCTCTGACATACTACGACT | |
| | | ACGAGTTCGCCTACTGGGGCCAGGGAACACTGGTGACCGTGTCCGCTGCTAGCACCAAGGGC | |
| | | CCATCGGTCTTCCCCCTGGCACCCTCCTCCAAGAGCACCTCTGGGGGCACAGCGGCCCTGGG | |
| | | CTGCCTGGTCAAGGACTACTTCCCCGAACCGGTGACGGTGTCGTGGAACTCAGGCGCCCTGA | |
| | | CCAGCGGCGTGCACACCTTCCCGGCTGTCCTACAGTCCTCAGGACTCTACTCCCTCAGCAGC | |
| | | GTGGTGACCGTGCCCTCCAGCAGCTTGGGCACCCAGACCTACATCTGCAACGTGAATCACAA | |
| | | GCCCAGCAACACCAAGGTGGACAAGAAAGTTGAGCCCAAATCTTGTGACAAAACTCACACAT | |
| | | GCCCACCGTGCCCAGCACCTGAACTCCTGGGGGGACCGTCAGTCTTCCTCTTCCCCCCAAAA | |
| | | CCCAAGGACACCCTCATGATCTCCCGGACCCCCGAGGTCACATGCGTGGTGGTGGACGTGAG | |
| | | CCACGAAGACCCTGAGGTCAAGTTCAACTGGTACGTGGACGGCGTGGAGGTGCATAATGCCA | |
| | | AGACAAAGCCGCGGGAGGAGCAGTACAACAGCACGTACCGTGTGGTCAGCGTCCTCACCGTC | |
| | | CTGCACCAGGACTGGCTGAATGGCAAGGAGTACAAGTGCAAGGTCTCCAACAAAGCCCTCCC | |
| | | AGCCCCCATCGAGAAAACCATCTCCAAAGCCAAAGGGCAGCCCCGAGAACCACAGGTGTACA | |
| | | CCCTGCCCCCATCCCGGGAGGAGATGACCAAGAACCAGGTCAGCCTGTGGTGCCTGGTCAAA | |
| | | GGCTTCTATCCCAGCGACATCGCCGTGGAGTGGGAGAGCAATGGGCAGCCGGAGAACAACTA | |
| | | CAAGACCACGCCTCCCGTGCTGGACTCCGACGGCTCCTTCTTCCTCTACAGCAAGCTCACCG | |
| | | TGGACAAGAGCAGGTGGCAGCAGGGGAACGTCTTCTCATGCTCCGTGATGCATGAGGCTCTG | |
| | | CACAACCACTACACGCAGAAGAGCCTCTCCCTGTCTCCGGGTAAAGGAGGCGGGGGATCTGG | |
| | | GGGAGGAGGTAGTGGAGGCGGAGGATCCGGCGGTGGAGGTTCTGGTGGTGGGGGCAGTGGGG | |
| | | GTGGCGGATCAGCTGCTGAACCTGTGGAAGATAATGTGATCAATTTCGTGGCCATGAAGTTC | |
| | | ATTGACAACACCCTGTACTTCATCGCTGAAGATGATGAAAACATCGAGCAGGACTATTTCGG | |
| | | AAAACTGGAAAGCAAACTGAGCGTGATCAGGAACCTGAACGACCAGGTGCTGTTTATCGACC | |
| | | AGGGCAACAGGCCCCTGTTCGAAGATATGACCGACAGCGACGTCAGGGACAACGCTCCTAGA | |
| | | ACAATCTTCATCATCTCCAAGTACGGCGACTCCCAGCCAAGGGGACTGGCTGTGACCATTTC | |
| | | CGTGAAAGTGGAGAAGATCTCCACACTGAGCGTGGAAAACAAAATCATCTCCTTCAAGGAGA | |
| | | TGAACCCCCCCGACAACATTAAAGACACCAAAAGCGATATCATCTTCTTCCAGAGGAGTGTG | |
| | | CCCGGCCACGATAACAAAGTGCAGTTCGAGAGCAGCTCCTACGAGGGATACTTTCTGGCCGT | |
| | | CGAAAAGGAGAGGGACCTGTTCAAGCTGATCCTGAAGAAGGAGGACGAGCTGGGGGACAGAT | |
| | | CAATCATGTTCACCGTGCAGAACGAAGAC (SEQ ID NO: 308) | |
| FUSE-685 | (SEQ ID NO: 114) | GAAGTGCAGCTGGTGGAAAGCGGAGGAGGATTGGTGCAGGCAGGAGGAAGTCTGAGGCTGAG | — |
| | | TTGTGCCGCTAGCGGTAGAACATTTAGTAGTTATGCTATGGGGTGGTTTAGGCAGGCTCCTG | |
| | | GAAAGGAGAGGGAGTTCGTGGTGGCTATCAACTGGAGCAGCGGCTCTACATACTACGCTGAT | |
| | | TCCGTGAAGGGAAGGTTCACAATCTCCAGGGACAATGCTAAGAACACAATGTATCTGCAGAT | |
| | | GAACAGCCTCAAGCCTGAGGACACCGCCGTTTACTATTGCGCCGCTGGATACCAAATCAACA | |
| | | GCGGCAACTACAACTTCAAGGACTATGAGTATGACTACTGGGGCCAGGGCACACAGGTGACA | |
| | | GTTTCATCCGAGCCCAAATCTTCTGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAAGCTGCTGAACCTGTGGAAGATAATTCTATTAATTTCGTG | |
| | | GCCATGAAATTCATCGATAATACTCTGTACTTCATCGCCGAAGATGATGAAAATCAGGATAT | |
| | | TGAATACTTCGGAAAACTGGAATCCAAATTGAGCGTGATCCGGAACCTGAACGATCAGGTGC | |
| | | TGTTCATCGACCAGGGCAATCGGCCACTCTTCGAGGACATGACCGACTCAGACTCCAGGGAC | |
| | | AACGCTCCCCGAACCATCTTCATCATCTCCAAGTACGGCGACTCCCAGCCCAGGGGATTGGC | |
| | | TGTTACCATTTCTGTGAAGTCTGAGAAGATCTCCACCCTGTCCTCCGAGAACAAAATCATCT | |
| | | CCTTCAAGGAGATGAACCCCCCCGACAACATCAAGGACACCAAGAGCGACATCATTTTTTTC | |
| | | CAGCGCTCCGTGCCCGGTCACGACAATAAGGTCCAGTTCGAATCCTCCAGCTACGAGGGCTA | |
| | | CTTTCTGGCCTCCGAAAAGGAAAGAGACTTGTTCAAGCTGATTCTGAAAAAGGAGGACGAGC | |
| | | TGGGCGACAGGTCCATCATGTTCACCGTGCAGAACGAGGAC (SEQ ID NO: 309) | |
| FUSE—686 | (SEQ ID NO: 114) | GAAGTGCAGCTGGTGGAAAGCGGAGGAGGATTGGTGCAGGCAGGAGGAAGTCTGAGGCTGAG | — |
| | | TTGTGCCGCTAGCGGTAGAACATTTAGTAGTTATGCTATGGGGTGGTTTAGGCAGGCTCCTG | |
| | | GAAAGGAGAGGGAGTTCGTGGTGGCTATCAACTGGAGCAGCGGCTCTACATACTACGCTGAT | |
| | | TCCGTGAAGGGAAGGTTCACAATCTCCAGGGACAATGCTAAGAACACAATGTATCTGCAGAT | |
| | | GAACAGCCTCAAGCCTGAGGACACCGCCGTTTACTATTGCGCCGCTGGATACCAAATCAACA | |
| | | GCGGCAACTACAACTTCAAGGACTATGAGTATGACTACTGGGGCCAGGGCACACAGGTGACA | |
| | | GTTTCATCCGAGCCCAAATCTTCTGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAAGCTGCTGAACCTGTGGAAGATAATTCTATTAATTTCGTG | |
| | | GCCATGAAATTCATCGATAATACTCTGTACTTCATCGCCGAAAATGATGAAAACCAGGACAT | |
| | | TGAATACTTTGGAAAACTGGAATCCAAATTGAGTGTGATCCGCAACCTGAACGATCAGGTGC | |
| | | TGTTCATCGACCAGGGCAATCGCCCCCTGTTCGAGGATATGACCGACTCCGACTCCAGGGAC | |
| | | AACGCACCTAGGACCATCTTCATCATCTCTAAGTACGGCGATTCCCAGCCCAGGGGCTTGGC | |
| | | TGTTACCATCTCCGTTAAGTCCGAGAAGATTTCCACCCTGTCCTCCGAGAACAAAATCATCT | |
| | | CCTTCAAAGAAATGAACCCCCCCGACAACATCAAGGACACCAAAAGCGATATCATCTTCTTC | |
| | | CAGAGGTCCGTGCCCGGCCACGATAACAAAGTGCAGTTCGAGTCCAGCTCCTACGAGGGCTA | |
| | | CTTCCTGGCCTCCGAAAAGGAACGCGACCTGTTCAAGCTGATCCTGAAGAAGGAGGACGAGC | |
| | | TGGGGGACAGATCCATTATGTTCACCGTTCAGAACGAGGAC (SEQ ID NO: 168) | |
| FUSE-691 | CAGGTTCAGCTCGTCGAGTCAGGCGGAGGAGTC | | SEQ |
| | GTTCAGCCAGGAAGGAGTCTCAGGCTGGACTGC | | ID |
| | AAGGCATCAGGCATCACATTCTCCAACTCCGGC | | NO: 188 |
| | ATGCACTGGGTGAGGCAGGCACCTGGAAAAGGA | | |
| | TTGGAGTGGGTGGCAGTGATCTGGTACGATGGA | | |
| | AGCAAAAGATACTACGCCGACAGCGTCAAGGGC | | |
| | AGGTTCACCATCAGCAGGGACAATAGCAAGAAC | | |
| | ACCCTGTTCTTGCAGATGAATAGCTTGAGGGCC | | |
| | GAGGACACCGCCGTGTATTACTGCGCTACCAAC | | |
| | GACGACTACTGGGGCCAGGGAACCCTGGTTACC | | |
| | GTGAGTAGCGCTAGCACCAAGGGCCCATCGGTC | | |
| | TTCCCCCTGGCACCCTCCTCCAAGAGCACCTCT | | |
| | GGGGGCACAGCGGCCCTGGGCTGCCTGGTCAAG | | |
| | GACTACTTCCCCGAACCGGTGACGGTGTCGTGG | | |
| | AACTCAGGCGCCCTGACCAGCGGCGTGCACACC | | |
| | TTCCCGGCTGTCCTACAGTCCTCAGGACTCTAC | | |
| | TCCCTCAGCAGCGTGGTGACCGTGCCCTCCAGC | | |
| | AGCTTGGGCACCCAGACCTACATCTGCAACGTG | | |
| | AATCACAAGCCCAGCAACACCAAGGTGGACAAG | | |
| | AAAGTTGAGCCCAAATCTTGTGACAAAACTCAC | | |
| | ACATGCCCACCGTGCCCAGCACCTGAAGCCGCA | | |
| | GGGGGACCGTCAGTCTTCCTCTTCCCCCCAAAA | | |
| | CCCAAGGACACCCTCATGATCTCCCGGACCCCC | | |
| | GAGGTCACATGCGTGGTGGTGGACGTGAGCCAC | | |
| | GAAGACCCTGAGGTCAAGTTCAACTGGTACGTG | | |
| | GACGGCGTGGAGGTGCATAATGCCAAGACAAAG | | |
| | CCGCGGGAGGAGCAGTACAACAGCACGTACCGT | | |
| | GTGGTCAGCGTCCTCACCGTCCTGCACCAGGAC | | |
| | TGGCTGAATGGCAAGGAGTACAAGTGCAAGGTC | | |
| | TCCAACAAAGCCCTCCCAGCCCCCATCGAGAAA | | |
| | ACCATCTCCAAAGCCAAAGGGCAGCCCCGAGAA | | |
| | CCACAGGTGTACACCCTGCCCCCATCCCGGGAG | | |
| | GAGATGACCAAGAACCAGGTCAGCCTGACCTGC | | |
| | CTGGTCAAAGGCTTCTATCCCAGCGACATCGCC | | |
| | GTGGAGTGGGAGAGCAATGGGCAGCCGGAGAAC | | |
| | AACTACAAGACCACGCCTCCCGTGCTGGACTCC | | |
| | GACGGCTCCTTCTTCCTCTACAGCAAGCTCACC | | |
| | GTGGACAAGAGCAGGTGGCAGCAGGGGAACGTC | | |
| | TTCTCATGCTCCGTGATGCATGAGGCTCTGCAC | | |
| | AACCACTACACGCAGAAGAGCCTCTCCCTGTCT | | |
| | CCGGGTAAA (SEQ ID NO: 129) | | |
| FUSE-694 | CAGGTTCAGCTCGTCGAAAGCGGAGGAGGAGTC | CAGGTTCAGCTCGTCGAAAGCGGAGGAGGAGTCGTTCAGCCAGGAAGATCCCTCAGGCTCGA | SEQ |
| | GTTCAGCCAGGAAGATCCCTCAGGCTCGACTGT | CTGTAAGGCCTCCGGAATCACCTTCTCCAACTCCGGAATGCACTGGGTCAGGCAGGCTCCTG | ID |
| | AAGGCCTCCGGAATCACCTTCTCCAACTCCGGA | GAAAAGGACTGGAGTGGGTGGCTGTGATTTGGTACGACGGCTCCAAGAGATACTACGCCGAC | NO: 188 |
| | ATGCACTGGGTCAGGCAGGCTCCTGGAAAAGGA | TCTGTGAAGGGCCGGTTCACTATCTCCAGGGACAACTCCAAGAACACACTGTTCCTGCAGAT | |
| | CTGGAGTGGGTGGCTGTGATTTGGTACGACGGC | GAACTCCCTGCGGGCTGAGGATACTGCCGTTTATTACTGCGCCACCAACGATGACTACTGGG | |
| | TCCAAGAGATACTACGCCGACTCTGTGAAGGGC | GACAGGGAACCTTGGTGACCGTGAGTAGCGCTAGCACCAAGGGCCCATCGGTCTTCCCCCTG | |
| | CGGTTCACTATCTCCAGGGACAACTCCAAGAAC | GCACCCTCCTCCAAGAGCACCTCTGGGGGCACAGCGGCCCTGGGCTGCCTGGTCAAGGACTA | |
| | ACACTGTTCCTGCAGATGAACTCCCTGCGGGCT | CTTCCCCGAACCGGTGACGGTGTCGTGGAACTCAGGCGCCCTGACCAGCGGCGTGCACACCT | |
| | GAGGATACTGCCGTTTATTACTGCGCCACCAAC | TCCCGGCTGTCCTACAGTCCTCAGGACTCTACTCCCTCAGCAGCGTGGTGACCGTGCCCTCC | |
| | GATGACTACTGGGGACAGGGAACCTTGGTGACC | AGCAGCTTGGGCACCCAGACCTACATCTGCAACGTGAATCACAAGCCCAGCAACACCAAGGT | |
| | GTGAGTAGCGCTAGCACCAAGGGCCCATCGGTC | GGACAAGAAAGTTGAGCCCAAATCTTGTGACAAAACTCACACATGCCCACCGTGCCCAGCAC | |
| | TTCCCCCTGGCACCCTCCTCCAAGAGCACCTCT | CTGAAGCCGCAGGGGGACCGTCAGTCTTCCTCTTCCCCCCAAAACCCAAGGACACCCTCATG | |
| | GGGGGCACAGCGGCCCTGGGCTGCCTGGTCAAG | ATCTCCCGGACCCCCGAGGTCACATGCGTGGTGGTGGACGTGAGCCACGAAGACCCTGAGGT | |
| | GACTACTTCCCCGAACCGGTGACGGTGTCGTGG | CAAGTTCAACTGGTACGTGGACGGCGTGGAGGTGCATAATGCCAAGACAAAGCCGCGGGAGG | |
| | AACTCAGGCGCCCTGACCAGCGGCGTGCACACC | AGCAGTACAACAGCACGTACCGTGTGGTCAGCGTCCTCACCGTCCTGCACCAGGACTGGCTG | |
| | TTCCCGGCTGTCCTACAGTCCTCAGGACTCTAC | AATGGCAAGGAGTACAAGTGCAAGGTCTCCAACAAAGCCCTCCCAGCCCCCATCGAGAAAAC | |
| | TCCCTCAGCAGCGTGGTGACCGTGCCCTCCAGC | CATCTCCAAAGCCAAAGGGCAGCCCCGAGAACCACAGGTGTACACCCTGCCCCCATCCCGGG | |
| | AGCTTGGGCACCCAGACCTACATCTGCAACGTG | AGGAGATGACCAAGAACCAGGTCAGCCTGTGGTGCCTGGTCAAAGGCTTCTATCCCAGCGAC | |
| | AATCACAAGCCCAGCAACACCAAGGTGGACAAG | ATCGCCGTGGAGTGGGAGAGCAATGGGCAGCCGGAGAACAACTACAAGACCACGCCTCCCGT | |
| | AAAGTTGAGCCCAAATCTTGTGACAAAACTCAC | GCTGGACTCCGACGGCTCCTTCTTCCTCTACAGCAAGCTCACCGTGGACAAGAGCAGGTGGC | |
| | ACATGCCCACCGTGCCCAGCACCTGAAGCCGCA | AGCAGGGGAACGTCTTCTCATGCTCCGTGATGCATGAGGCTCTGCACAACCACTACACGCAG | |
| | GGGGGACCGTCAGTCTTCCTCTTCCCCCCAAAA | AAGAGCCTCTCCCTGTCTCCGGGTAAA (SEQ ID NO: 169) | |
| | CCCAAGGACACCCTCATGATCTCCCGGACCCCC | | |
| | GAGGTCACATGCGTGGTGGTGGACGTGAGCCAC | | |
| | GAAGACCCTGAGGTCAAGTTCAACTGGTACGTG | | |
| | GACGGCGTGGAGGTGCATAATGCCAAGACAAAG | | |
| | CCGCGGGAGGAGCAGTACAACAGCACGTACCGT | | |
| | GTGGTCAGCGTCCTCACCGTCCTGCACCAGGAC | | |
| | TGGCTGAATGGCAAGGAGTACAAGTGCAAGGTC | | |
| | TCCAACAAAGCCCTCCCAGCCCCCATCGAGAAA | | |
| | ACCATCTCCAAAGCCAAAGGGCAGCCCCGAGAA | | |
| | CCACAGGTGTACACCCTGCCCCCATCCCGGGAG | | |
| | GAGATGACCAAGAACCAGGTCAGCCTGAGCTGC | | |
| | GCCGTCAAAGGCTTCTATCCCAGCGACATCGCC | | |
| | GTGGAGTGGGAGAGCAATGGGCAGCCGGAGAAC | | |
| | AACTACAAGACCACGCCTCCCGTGCTGGACTCC | | |
| | GACGGCTCCTTCTTCCTCGTGAGCAAGCTCACC | | |
| | GTGGACAAGAGCAGGTGGCAGCAGGGGAACGTC | | |
| | TTCTCATGCTCCGTGATGCATGAGGCTCTGCAC | | |
| | AACAGGTTCACACAGAAGAGCTTGAGCTTGAGC | | |
| | CCCGGCGGAGGCGGGGGATCTGGGGGAGGAGGT | | |
| | AGTGGAGGCGGAGGATCCGGCGGTGGAGGTTCT | | |
| | GGTGGTGGGGGCAGTGGGGGTGGCGGATCAGCT | | |
| | GCTGAACCTGTGGAAGATAATTCTATTAACTTC | | |
| | GTGGCCATGAAGTTCATCGATAATACTCTGTAC | | |
| | TTCATCGCCGAAGATGATGAAAATATCGAGCAG | | |
| | GATTACTTCGGCAAACTGGAAAGCAAACTGAGC | | |
| | GTGATCAGGAACCTCAACGACCAGGTGCTGTTC | | |
| | ATCGATCAGGGCAACAGGCCCCTCTTTGAGGAC | | |
| | ATGACCGACAGCGATAGCAGGGACAACGCACCT | | |
| | AGGACAATCTTCATCATCTCCAAGTACGGCGAC | | |
| | TCCCAGCCCAGAGGATTGGCTGTGACAATTTCC | | |
| | GTGAAATCCGAGAAGATCTCCACCCTGTCCAGC | | |
| | GAGAACAAGATCATCTCCTTCAAGGAGATGAAC | | |
| | CCCCCCGACAACATCAAGGACACAAAGTCCGAC | | |
| | ATCATCTTCTTCCAGCGCTCCGTGCCCGGTCAT | | |
| | GATAACAAGGTCCAGTTCGAGTCCAGCTCCTAC | | |
| | GAGGGATACTTTCTGGCCTCAGAGAAGGAAAGG | | |
| | GACCTGTTCAAACTCATCCTGAAGAAGGAGGAC | | |
| | GAGCTGGGGGACAGAAGCATCATGTTCACCGTC | | |
| | CAGAATGAGGAC (SEQ ID NO: 130) | | |
| FUSE-756 | (SEQ ID NO: 114) | GGAGGCGGGGGATCTGGGGGAGGAGGTAGTGGAGGCGGAGGATCCGGCGGTGGAGGTTCTGG | |
| | | TGGTGGGGGCAGTGGGGGTGGCGGATCAGGAATTGAGCAGGACTACTTTGGAAAGCTGGAGA | |
| | | GCAAGCTGAGCGTGATCAGGAACCTGAACGATCAGGTGCTGTTCATCGATCAGGGAAACAGG | |
| | | CCTCTGTTTGAGGACATGACAGACAGCGACGTCAGGGACAACGCACCCAGAACCATCTTCAT | |
| | | CATCTCCATGTACAAGGACTCCCAGCCCAGGGGAATGGCCGTTACAATTTCCGTGAAAGTGG | |
| | | AGAAGATCTCCACACTGAGCGTGGAGAACAAGATCATCTCCTTCAAGGAGATGAACCCCCCC | |
| | | GACAACATCAAGGACACCAAATCCGACATCATCTTCTTCCAGCGCTCCGTGCCCGGGCATGA | |
| | | TAACAAGATGCAGTTCGAAAGCTCCTCCTACGAAGGCTACTTCCTCGCCGTCGAAAAGGAAA | |
| | | GGGACCTGTTCAAGCTCATCCTCAAGAAGGAGGACGAGCTGGGGGACAGAAGCATCATGTTC | |
| | | ACCGTGCAGAACGAAGACGAACCTAAATCCTCCGACAAAACTCACACATGCCCACCGTGCCC | |
| | | AGCACCTGAACTCCTGGGGGGACCGTCAGTCTTCCTCTTCCCCCCAAAACCCAAGGACACCC | |
| | | TCATGATCTCCCGGACCCCCGAGGTCACATGCGTGGTGGTGGACGTGAGCCACGAAGACCCT | |
| | | GAGGTCAAGTTCAACTGGTACGTGGACGGCGTGGAGGTGCATAATGCCAAGACAAAGCCGCG | |
| | | GGAGGAGCAGTACAACAGCACGTACCGTGTGGTCAGCGTCCTCACCGTCCTGCACCAGGACT | |
| | | GGCTGAATGGCAAGGAGTACAAGTGCAAGGTCTCCAACAAAGCCCTCCCAGCCCCCATCGAG | |
| | | AAAACCATCTCCAAAGCCAAAGGGCAGCCCCGAGAACCACAGGTGTACACCCTGCCCCCATC | |
| | | CCGGGAGGAGATGACCAAGAACCAGGTCAGCCTGTGGTGCCTGGTCAAAGGCTTCTATCCCA | |
| | | GCGACATCGCCGTGGAGTGGGAGAGCAATGGGCAGCCGGAGAACAACTACAAGACCACGCCT | |
| | | CCCGTGCTGGACTCCGACGGCTCCTTCTTCCTCTACAGCAAGCTCACCGTGGACAAGAGCAG | |
| | | GTGGCAGCAGGGGAACGTCTTCTCATGCTCCGTGATGCATGAGGCTCTGCACAACCACTACA | |
| | | CGCAGAAGAGCCTCTCCCTGTCTCCGGGTAAA (SEQ ID NO: 170) | |
| FUSE-757 | (SEQ ID NO: 114) | GGTGGAGGAGGTTCTGGAGGTGGTGGATCAGGAATTGAGCAGGACTACTTTGGAAAGCTGGA | |
| | | GAGCAAGCTGAGCGTGATCAGGAACCTGAACGATCAGGTGCTGTTCATCGATCAGGGAAACA | |
| | | GGCCTCTGTTTGAGGACATGACAGACAGCGACGTCAGGGACAACGCACCCAGAACCATCTTC | |
| | | ATCATCTCCATGTACAAGGACTCCCAGCCCAGGGGAATGGCCGTTACAATTTCCGTGAAAGT | |
| | | GGAGAAGATCTCCACACTGAGCGTGGAGAACAAGATCATCTCCTTCAAGGAGATGAACCCCC | |
| | | CCGACAACATCAAGGACACCAAATCCGACATCATCTTCTTCCAGCGCTCCGTGCCCGGGCAT | |
| | | GATAACAAGATGCAGTTCGAAAGCTCCTCCTACGAAGGCTACTTCCTCGCCGTCGAAAAGGA | |
| | | AAGGGACCTGTTCAAGCTCATCCTCAAGAAGGAGGACGAGCTGGGGGACAGAAGCATCATGT | |
| | | TCACCGTGCAGAACGAAGACGAACCTAAATCCTCCGACAAAACTCACACATGCCCACCGTGC | |
| | | CCAGCACCTGAACTCCTGGGGGGACCGTCAGTCTTCCTCTTCCCCCCAAAACCCAAGGACAC | |
| | | CCTCATGATCTCCCGGACCCCCGAGGTCACATGCGTGGTGGTGGACGTGAGCCACGAAGACC | |
| | | CTGAGGTCAAGTTCAACTGGTACGTGGACGGCGTGGAGGTGCATAATGCCAAGACAAAGCCG | |
| | | CGGGAGGAGCAGTACAACAGCACGTACCGTGTGGTCAGCGTCCTCACCGTCCTGCACCAGGA | |
| | | CTGGCTGAATGGCAAGGAGTACAAGTGCAAGGTCTCCAACAAAGCCCTCCCAGCCCCCATCG | |
| | | AGAAAACCATCTCCAAAGCCAAAGGGCAGCCCCGAGAACCACAGGTGTACACCCTGCCCCCA | |
| | | TCCCGGGAGGAGATGACCAAGAACCAGGTCAGCCTGTGGTGCCTGGTCAAAGGCTTCTATCC | |
| | | CAGCGACATCGCCGTGGAGTGGGAGAGCAATGGGCAGCCGGAGAACAACTACAAGACCACGC | |
| | | CTCCCGTGCTGGACTCCGACGGCTCCTTCTTCCTCTACAGCAAGCTCACCGTGGACAAGAGC | |
| | | AGGTGGCAGCAGGGGAACGTCTTCTCATGCTCCGTGATGCATGAGGCTCTGCACAACCACTA | |
| | | CACGCAGAAGAGCCTCTCCCTGTCTCCGGGTAAA (SEQ ID NO: 171) | |
| FUSE-758 | (SEQ ID NO: 114) | TTGCAGGCTCGTGTTGTGGGGGGTGGTGGTACTATTGAGCAGGACTACTTTGGGAAGCTGGA | |
| | | GAGCAAGTTGAGCGTTATTAGGAATCTGAACGACCAGGTTTTGTTTATCGACCAGGGGAACC | |
| | | GTCCTTTGTTTGAGGACATGACAGACAGCGACGTCAGGGACAACGCTCCAAGGACAATCTTC | |
| | | ATCATCTCCATGTACAAGGACTCCCAGCCCAGGGGAATGGCCGTTACAATCAGCGTGAAGGT | |
| | | GGAGAAAATCAGCACACTGAGCGTGGAGAACAAGATCATCAGCTTCAAGGAAATGAACCCCC | |
| | | CCGACAACATCAAGGACACCAAATCCGACATCATCTTTTTCCAGAGGAGTGTGCCCGGACAC | |
| | | GACAACAAAATGCAGTTCGAATCCAGCTCCTACGAGGGCTACTTCCTGGCCGTCGAAAAGGA | |
| | | AAGGGACCTGTTCAAGCTGATCCTGAAGAAGGAGGATGAGCTGGGGGACAGGTCCATCATGT | |
| | | TCACCGTGCAGAACGAGGACGAACCTAAATCCTCCGACAAAACTCACACATGCCCACCGTGC | |
| | | CCAGCACCTGAACTCCTGGGGGGACCGTCAGTCTTCCTCTTCCCCCCAAAACCCAAGGACAC | |
| | | CCTCATGATCTCCCGGACCCCCGAGGTCACATGCGTGGTGGTGGACGTGAGCCACGAAGACC | |
| | | CTGAGGTCAAGTTCAACTGGTACGTGGACGGCGTGGAGGTGCATAATGCCAAGACAAAGCCG | |
| | | CGGGAGGAGCAGTACAACAGCACGTACCGTGTGGTCAGCGTCCTCACCGTCCTGCACCAGGA | |
| | | CTGGCTGAATGGCAAGGAGTACAAGTGCAAGGTCTCCAACAAAGCCCTCCCAGCCCCCATCG | |
| | | AGAAAACCATCTCCAAAGCCAAAGGGCAGCCCCGAGAACCACAGGTGTACACCCTGCCCCCA | |
| | | TCCCGGGAGGAGATGACCAAGAACCAGGTCAGCCTGTGGTGCCTGGTCAAAGGCTTCTATCC | |
| | | CAGCGACATCGCCGTGGAGTGGGAGAGCAATGGGCAGCCGGAGAACAACTACAAGACCACGC | |
| | | CTCCCGTGCTGGACTCCGACGGCTCCTTCTTCCTCTACAGCAAGCTCACCGTGGACAAGAGC | |
| | | AGGTGGCAGCAGGGGAACGTCTTCTCATGCTCCGTGATGCATGAGGCTCTGCACAACCACTA | |
| | | CACGCAGAAGAGCCTCTCCCTGTCTCCGGGTAAA (SEQ ID NO: 172) | |
| FUSE-775 | (SEQ ID NO: 114) | GAAGTGCAGCTGGTGGAGAGCGGAGGAGGATTGGTGCAGGCAGGAGGAAGCCTGAGGCTGAG | |
| | | TTGTGCCGCTAGTGGAAGGACATTTAGTTCCTATGCAATGGGGTGGTTCAGGCAGGCCCCAG | |
| | | GAAAGGAAAGGGAGTTTGTGGTGGCCATCAACTGGAGTAGCGGATCTACATACTACGCTGAT | |
| | | TCTGTGAAGGGCAGGTTCACCATCAGCCGGGACAATGCTAAGAACACCATGTACCTGCAGAT | |
| | | GTCCTCCCTGAAGCCTGAAGATACTGCTGTGTACTACTGCGCCGCCGGCTATCAGATCAATT | |
| | | CCGGAAACTACAACTTCAAAGATTACGAGTATGACTACTGGGGCCAGGGTACCCAGGTGACA | |
| | | GTGAGTTCCGAACCTAAATCCTCCGACAAAACTCACACATGCCCACCGTGCCCAGCACCTGA | |
| | | ACTCCTGGGGGGACCGTCAGTCTTCCTCTTCCCCCCAAAACCCAAGGACACCCTCATGATCT | |
| | | CCCGGACCCCCGAGGTCACATGCGTGGTGGTGGACGTGAGCCACGAAGACCCTGAGGTCAAG | |
| | | TTCAACTGGTACGTGGACGGCGTGGAGGTGCATAATGCCAAGACAAAGCCGCGGGAGGAGCA | |
| | | GTACAACAGCACGTACCGTGTGGTCAGCGTCCTCACCGTCCTGCACCAGGACTGGCTGAATG | |
| | | GCAAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATCCCGGGAGGA | |
| | | GATGACCAAGAACCAGGTCAGCCTGTGGTGCCTGGTCAAAGGCTTCTATCCCAGCGACATCG | |
| | | CCGTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACTACACGCAGAAGA | |
| | | GCCTCTCCCTGTCTCCGGGTAAAGCTGCTGAACCTGTGGAAGATAATGTGATCAATTTTGTG | |
| | | GCCATGAAATTTATTGACAACACCCTGTATTTCATCGCCGAAAACGATGAAAACGGCGGAGG | |
| | | ATCTTATTTTGGAAAGCTGGAATCCAAATTGAGCGTGATCAGGAACCTGAACGATCAGGTGC | |
| | | TGTTTATCGACCAGGGCAACAGGCCCCTGTTCGAGGATATGACCGACAGTGACGTGAGGGAC | |
| | | AACGCTCCCCGAACCATCTTCATCATCAGTAAGTATGGAGACTCCCAGCCAAGGGGACTGGC | |
| | | CGTTACAATTAGCGTGAAAGTCGAGAAGATTTCCACATTGAGCGTGGAGAACAAGATCATCT | |
| | | CCTTCAAGGAGATGAACCCCCCCGACAACATTAAGGACACCAAAAGCGATATCATCTTCTTC | |
| | | CAGAGGTCCGTGCCTGGGCACGACAATAAAGTGCAGTTCGAGTCCAGCTCCTACGAGGGCTA | |
| | | CTTTCTGGCAGTGGAGAAGGAAAGGGACCTGTTCAAGCTGATCCTGAAGAAGGAGGACGAGC | |
| | | TGGGGGACAGGTCCATCATGTTCACCGTCCAGAATGAGGAC (SEQ ID NO: 173) | |
| FUSE-782 | CAGGTTCAGCTCGTCGAAAGCGGAGGAGGAGTC | CAGGTTCAACTCGTCGAGTCAGGCGGAGGAGTCGTTCAACCAGGAAGAAGCCTCAGACTCGA | SEQ |
| | GTTCAGCCAGGAAGATCCCTCAGGCTCGACTGT | CTGCAAAGCAAGCGGCATCACCTTCAGCAACAGCGGAATGCACTGGGTGAGACAAGCCCCTG | ID |
| | AAGGCCTCCGGAATCACCTTCTCCAACTCCGGA | GAAAAGGCCTGGAGTGGGTTGCTGTGATTTGGTACGACGGCAGCAAAAGATATTACGCCGAC | NO: 188 |
| | ATGCACTGGGTCAGGCAGGCTCCTGGAAAAGGA | AGTGTGAAAGGCAGATTTACAATCTCAAGGGACAACAGCAAGAACACACTGTTCCTGCAGAT | |
| | CTGGAGTGGGTGGCTGTGATTTGGTACGACGGC | GAGCAGCCTGAGAGCCGAGGATACTGCAGTGTATTACTGCGCCACCAACGACGACTACTGGG | |
| | TCCAAGAGATACTACGCCGACTCTGTGAAGGGC | GACAAGGAACCCTGGTGACCGTGAGTAGCGCTAGCACCAAGGGCCCATCGGTCTTCCCCCTG | |
| | CGGTTCACTATCTCCAGGGACAACTCCAAGAAC | GCACCCTCCTCCAAGAGCACCTCTGGGGGCACAGCGGCCCTGGGCTGCCTGGTCAAGGACTA | |
| | ACACTGTTCCTGCAGATGAGCAGTTTGAGGGCC | CTTCCCCGAACCGGTGACGGTGTCGTGGAACTCAGGCGCCCTGACCAGCGGCGTGCACACCT | |
| | GAGGACACCGCTGTTTACTACTGCGCAACCAAC | TCCCGGCTGTCCTACAGTCCTCAGGACTCTACTCCCTCAGCAGCGTGGTGACCGTGCCCTCC | |
| | GATGACTACTGGGGGCAGGGAACCCTGGTTACA | AGCAGCTTGGGCACCCAGACCTACATCTGCAACGTGAATCACAAGCCCAGCAACACCAAGGT | |
| | GTGAGTAGCGCTAGCACCAAGGGCCCATCGGTC | GGACAAGAAAGTTGAGCCCAAATCTTGTGACAAAACTCACACATGCCCACCGTGCCCAGCAC | |
| | TTCCCCCTGGCACCCTCCTCCAAGAGCACCTCT | CTGAAGCCGCAGGGGGACCGTCAGTCTTCCTCTTCCCCCCAAAACCCAAGGACACCCTCATG | |
| | GGGGGCACAGCGGCCCTGGGCTGCCTGGTCAAG | ATCTCCCGGACCCCCGAGGTCACATGCGTGGTGGTGGACGTGAGCCACGAAGACCCTGAGGT | |
| | GACTACTTCCCCGAACCGGTGACGGTGTCGTGG | CAAGTTCAACTGGTACGTGGACGGCGTGGAGGTGCATAATGCCAAGACAAAGCCGCGGGAGG | |
| | AACTCAGGCGCCCTGACCAGCGGCGTGCACACC | AGCAGTACAACAGCACGTACCGTGTGGTCAGCGTCCTCACCGTCCTGCACCAGGACTGGCTG | |
| | TTCCCGGCTGTCCTACAGTCCTCAGGACTCTAC | AATGGCAAGGAGTACAAGTGCAAGGTCTCCAACAAAGCCCTCCCAGCCCCCATCGAGAAAAC | |
| | TCCCTCAGCAGCGTGGTGACCGTGCCCTCCAGC | CATCTCCAAAGCCAAAGGGCAGCCCCGAGAACCACAGGTGTACACCCTGCCCCCATCCCGGG | |
| | AGCTTGGGCACCCAGACCTACATCTGCAACGTG | AGGAGATGACCAAGAACCAGGTCAGCCTGTGGTGCCTGGTCAAAGGCTTCTATCCCAGCGAC | |
| | AATCACAAGCCCAGCAACACCAAGGTGGACAAG | ATCGCCGTGGAGTGGGAGAGCAATGGGCAGCCGGAGAACAACTACAAGACCACGCCTCCCGT | |
| | AAAGTTGAGCCCAAATCTTGTGACAAAACTCAC | GCTGGACTCCGACGGCTCCTTCTTCCTCTACAGCAAGCTCACCGTGGACAAGAGCAGGTGGC | |
| | ACATGCCCACCGTGCCCAGCACCTGAAGCCGCA | AGCAGGGGAACGTCTTCTCATGCTCCGTGATGCATGAGGCTCTGCACAACCACTACACGCAG | |
| | GGGGGACCGTCAGTCTTCCTCTTCCCCCCAAAA | AAGAGCCTCTCCCTGTCTCCGGGTGGTGGCGGAGGGTCTGGTGGTGGAGGATCCGGGGGAGG | |
| | CCCAAGGACACCCTCATGATCTCCCGGACCCCC | AGGTTCAGGAGGCGGGGGAAGTGCTGCTGAACCTGTGGAAGATAATTCTATTAACTTCGTGG | |
| | GAGGTCACATGCGTGGTGGTGGACGTGAGCCAC | CCATGAAGTTCATCGATAATACTCTGTACTTCATCGCCGAAAACGATGAGAACGGAGGAGGA | |
| | GAAGACCCTGAGGTCAAGTTCAACTGGTACGTG | TCTTACTTTGGAAAACTGGAAAGCAAGCTGAGCGTGATCCGGAACCTGAACGATCAAGTGCT | |
| | GACGGCGTGGAGGTGCATAATGCCAAGACAAAG | GTTTATCGACCAAGGCAACAGGCCCCTGTTCGAGGATATGACCGACAGCGACAGCAGGGACA | |
| | CCGCGGGAGGAGCAGTACAACAGCACGTACCGT | ACGCTCCTAGAACAATCTTCATCATCTCCAAGTACGGCGACAGCCAGCCCAGAGGACTGGCT | |
| | GTGGTCAGCGTCCTCACCGTCCTGCACCAGGAC | GTGACCATAAGCGTGAAAAGCGAGAAAATTAGCACCCTGAGCAGCGAGAACAAGATCATATC | |
| | TGGCTGAATGGCAAGGAGTACAAGTGCAAGGTC | CTTCAAAGAGATGAACCCCCCCGACAACATTAAAGACACCAAAAGCGATATCATCTTCTTCC | |
| | TCCAACAAAGCCCTCCCAGCCCCCATCGAGAAA | AGAGAAGCGTGCCCGGCCACGATAACAAAGTGCAATTCGAAAGCAGCAGCTACGAGGGCTAC | |
| | ACCATCTCCAAAGCCAAAGGGCAGCCCCGAGAA | TTCCTGGCTTCCGAAAAGGAGAGGGACCTGTTCAAGCTGATCCTGAAGAAGGAGGACGAGCT | |
| | CCACAGGTGTACACCCTGCCCCCATCCCGGGAG | GGGGGACAGATCAATAATGTTCACCGTGCAGAACGAGGAC (SEQ ID NO: 174) | |
| | GAGATGACCAAGAACCAGGTCAGCCTGAGCTGC | | |
| | GCCGTCAAAGGCTTCTATCCCAGCGACATCGCC | | |
| | GTGGAGTGGGAGAGCAATGGGCAGCCGGAGAAC | | |
| | AACTACAAGACCACGCCTCCCGTGCTGGACTCC | | |
| | GACGGCTCCTTCTTCCTCGTGAGCAAGCTCACC | | |
| | GTGGACAAGAGCAGGTGGCAGCAGGGGAACGTC | | |
| | TTCTCATGCTCCGTGATGCATGAGGCTCTGCAC | | |
| | AACAGGTTCACGCAGAAGAGCCTCTCCCTGTCT | | |
| | CCGGGTAAA (SEQ ID NO: 131) | | |
| FUSE-783 | (SEQ ID NO: 131) | CAGGTTCAACTCGTCGAGTCAGGCGGAGGAGTCGTTCAACCAGGAAGAAGCCTCAGACTCGA | SEQ |
| | | CTGCAAAGCAAGCGGCATCACCTTCAGCAACAGCGGAATGCACTGGGTGAGACAAGCCCCTG | ID |
| | | GAAAAGGCCTGGAGTGGGTTGCTGTGATTTGGTACGACGGCAGCAAAAGATATTACGCCGAC | NO: 188 |
| | | AGTGTGAAAGGCAGATTTACAATCTCAAGGGACAACAGCAAGAACACACTGTTCCTGCAGAT | |
| | | GAGCAGCCTGAGAGCCGAGGATACTGCAGTGTATTACTGCGCCACCAACGACGACTACTGGG | |
| | | GACAAGGAACCCTGGTGACCGTGAGTAGCGCTAGCACCAAGGGCCCATCGGTCTTCCCCCTG | |
| | | GCACCCTCCTCCAAGAGCACCTCTGGGGGCACAGCGGCCCTGGGCTGCCTGGTCAAGGACTA | |
| | | CTTCCCCGAACCGGTGACGGTGTCGTGGAACTCAGGCGCCCTGACCAGCGGCGTGCACACCT | |
| | | TCCCGGCTGTCCTACAGTCCTCAGGACTCTACTCCCTCAGCAGCGTGGTGACCGTGCCCTCC | |
| | | AGCAGCTTGGGCACCCAGACCTACATCTGCAACGTGAATCACAAGCCCAGCAACACCAAGGT | |
| | | GGACAAGAAAGTTGAGCCCAAATCTTGTGACAAAACTCACACATGCCCACCGTGCCCAGCAC | |
| | | CTGAAGCCGCAGGGGGACCGTCAGTCTTCCTCTTCCCCCCAAAACCCAAGGACACCCTCATG | |
| | | ATCTCCCGGACCCCCGAGGTCACATGCGTGGTGGTGGACGTGAGCCACGAAGACCCTGAGGT | |
| | | CAAGTTCAACTGGTACGTGGACGGCGTGGAGGTGCATAATGCCAAGACAAAGCCGCGGGAGG | |
| | | AGCAGTACAACAGCACGTACCGTGTGGTCAGCGTCCTCACCGTCCTGCACCAGGACTGGCTG | |
| | | AATGGCAAGGAGTACAAGTGCAAGGTCTCCAACAAAGCCCTCCCAGCCCCCATCGAGAAAAC | |
| | | CATCTCCAAAGCCAAAGGGCAGCCCCGAGAACCACAGGTGTACACCCTGCCCCCATCCCGGG | |
| | | AGGAGATGACCAAGAACCAGGTCAGCCTGTGGTGCCTGGTCAAAGGCTTCTATCCCAGCGAC | |
| | | ATCGCCGTGGAGTGGGAGAGCAATGGGCAGCCGGAGAACAACTACAAGACCACGCCTCCCGT | |
| | | GCTGGACTCCGACGGCTCCTTCTTCCTCTACAGCAAGCTCACCGTGGACAAGAGCAGGTGGC | |
| | | AGCAGGGGAACGTCTTCTCATGCTCCGTGATGCATGAGGCTCTGCACAACCACTACACGCAG | |
| | | AAGAGCCTCTCCCTGTCTCCGGGTGGTGGCGGAGGGTCTGGTGGTGGAGGATCCGGGGGAGG | |
| | | AGGTTCAGGAGGCGGGGGAAGTGCTGCTGAACCTGTGGAAGATAATGTGATAAACTTCGTGG | |
| | | CCATGAAGTTCATTGACAATACCCTGTATTTCATCGCCGAGAATGATGAAAACGGAGGAGGA | |
| | | TCTTACTTCGGAAAACTGGAAAGCAAACTGAGCGTGATAAGAAACCTGAACGATCAAGTGCT | |
| | | GTTCATCGACCAGGGCAACCGCCCTCTGTTCGAAGATATGACCGACAGCGACGTGAGGGACA | |
| | | ACGCTCCTAGAACAATCTTCATCATCAGCAAATACGGCGACTCCCAGCCCAGGGGACTGGCT | |
| | | GTGACCATCAGCGTGAAAGTGGAGAAGATAAGCACACTGAGCGTGGAGAACAAGATCATATC | |
| | | CTTTAAGGAGATGAACCCCCCCGACAACATCAAAGACACCAAAAGCGATATCATCTTCTTCC | |
| | | AGAGAAGCGTGCCTGGGCACGACAACAAAGTGCAGTTCGAGTCAAGCAGCTACGAGGGCTAC | |
| | | TTCCTCGCCGTTGAAAAGGAAAGAGACTTGTTCAAGCTGATCCTGAAGAAGGAGGACGAGCT | |
| | | GGGGGACAGAAGCATAATGTTCACCGTTCAGAACGAGGAC (SEQ ID NO: 175) | |
| FUSE-827 | GAGCCCAAATCTAGCGACAAAACTCACACATGC | GAGCCTAAAAGCAGCGACAAGACCCACACCTGCCCCCCTTGCCCCGCTCCTGAAGCTGCTGG | |
| | CCACCGTGCCCAGCACCTGAAGCCGCAGGGGGA | AGGCCCTTCAGTGTTTCTGTTTCCTCCCAAGCCCAAGGACACACTGATGATTTCCAGAACCC | |
| | CCGTCAGTCTTCCTCTTCCCCCCAAAACCCAAG | CCGAGGTGACATGCGTGGTGGTGGACGTGAGCCACGAAGATCCCGAGGTGAAGTTTAATTGG | |
| | GACACCCTCATGATCTCCCGGACCCCCGAGGTC | TACGTGGACGGCGTGGAAGTGCACAACGCCAAGACCAAACCCCGCGAGGAGCAGTATAATAG | |
| | ACATGCGTGGTGGTGGACGTGAGCCACGAAGAC | CACCTACAGAGTGGTGAGCGTGCTGACCGTGCTGCATCAGGACTGGCTGAACGGCAAGGAAT | |
| | CCTGAGGTCAAGTTCAACTGGTACGTGGACGGC | ACAAATGCAAGGTGAGCAACAAGGCCCTGCCTGCCCCTATCGAGAAGACCATCAGCAAAGCC | |
| | GTGGAGGTGCATAATGCCAAGACAAAGCCGCGG | AAGGGACAGCCAAGAGAACCCCAGGTGTACACCCTGCCCCCCAGCAGAGAAGAAATGACCAA | |
| | GAGGAGCAGTACAACAGCACGTACCGTGTGGTC | GAACCAGGTGAGCCTGTGGTGCCTGGTGAAAGGCTTCTACCCCAGCGACATCGCCGTGGAGT | |
| | AGCGTCCTCACCGTCCTGCACCAGGACTGGCTG | GGGAGAGCAACGGCCAGCCAGAGAACAACTACAAGACAACACCCCCCGTGCTGGACAGCGAC | |
| | AATGGCAAGGAGTACAAGTGCAAGGTCTCCAAC | GGCTCTTTCTTCCTGTACAGTAAACTGACCGTGGACAAATCAAGGTGGCAGCAGGGCAACGT | |
| | AAAGCCCTCCCAGCCCCCATCGAGAAAACCATC | GTTCAGCTGCTCCGTGATGCACGAGGCCCTGCACAACCACTATACCCAGAAGAGCCTGTCCC | |
| | TCCAAAGCCAAAGGGCAGCCCCGAGAACCACAG | TGAGCCCCGGCAGCGGAGGAGGAGGAGCTGCTGAACCTGTGGAAGATAATTCTATTAACTTC | |
| | GTGTACACCCTGCCCCCATCCCGGGAGGAGATG | GTGGCCATGAAGTTCATCGATAATACTCTGTACTTCATCGCCGAAAACGATGAGAACGGAGG | |
| | ACCAAGAACCAGGTCAGCCTGAGCTGCGCCGTC | AGGATCTTACTTTGGAAAACTGGAAAGCAAGCTGAGCGTGATCCGGAACCTGAACGATCAAG | |
| | AAAGGCTTCTATCCCAGCGACATCGCCGTGGAG | TGCTGTTTATCGACCAAGGCAACAGGCCCCTGTTCGAGGATATGACCGACAGCGACAGCAGG | |
| | TGGGAGAGCAATGGGCAGCCGGAGAACAACTAC | GACAACGCTCCTAGAACAATCTTCATCATCTCCAAGTACGGCGACAGCCAGCCCAGAGGACT | |
| | AAGACCACGCCTCCCGTGCTGGACTCCGACGGC | GGCTGTGACCATAAGCGTGAAAAGCGAGAAAATTAGCACCCTGAGCAGCGAGAACAAGATCA | |
| | TCCTTCTTCCTCGTGAGCAAGCTCACCGTGGAC | TATCCTTCAAAGAGATGAACCCCCCCGACAACATTAAAGACACCAAAAGCGATATCATCTTC | |
| | AAGAGCAGGTGGCAGCAGGGGAACGTCTTCTCA | TTCCAGAGAAGCGTGCCCGGCCACGATAACAAAGTGCAATTCGAAAGCAGCAGCTACGAGGG | |
| | TGCTCCGTGATGCATGAGGCTCTGCACAACCGG | CTACTTCCTGGCTTCCGAAAAGGAGAGGGACCTGTTCAAGCTGATCCTGAAGAAGGAGGACG | |
| | TTCACGCAGAAGAGCCTCTCCCTGTCTCCGGGT | AGCTGGGGGACAGATCAATAATGTTCACCGTGCAGAACGAGGAC (SEQ ID NO: 176) | |
| | AAA (SEQ ID NO: 132) | | |
| FUSE-832 | GAGCCCAAATCTAGCGACAAAACTCACACATGC | TACTTCGGCAAGCTGGAGTCCAAGCTGTCCGTGATCAGGAATCTCAACGACCAGGTGCTGTT | — |
| | CCACCGTGCCCAGCACCTGAAGCCGCAGGGGGA | CATCGACCAGGGGAACAGGCCCCTCTTCGAGGATATGACCGACTCCGACGTGAGGGATAACG | |
| | CCGTCAGTCTTCCTCTTCCCCCCAAAACCCAAG | CTCCCAGGACAATCTTCATCATTTCCAAGTACGGGGATTCCCAGCCAAGGGGACTGGCTGTT | |
| | GACACCCTCATGATCTCCCGGACCCCCGAGGTC | ACAATTTCCGTGAAGGTGGAGAAGATCAGCACACTCTCCGTGGAGAACAAGATCATCAGCTT | |
| | ACATGCGTGGTGGTGGACGTGAGCCACGAAGAC | CAAAGAGATGAACCCCCCCGACAACATTAAGGACACCAAAAGCGATATCATCTTCTTCCAGC | |
| | CCTGAGGTCAAGTTCAACTGGTACGTGGACGGC | GCTCCGTGCCTGGCCACGATAACAAGGTGCAGTTCGAGAGCTCCTCCTACGAAGGCTACTTT | |
| | GTGGAGGTGCATAATGCCAAGACAAAGCCGCGG | CTGGCCGTCGAAAAGGAGAGGGACCTGTTCAAGTTGATCCTGAAGAAGGAGGACGAGCTGGG | |
| | GAGGAGCAGTACAACAGCACGTACCGTGTGGTC | CGACAGGAGCATCATGTTCACCGTCCAGAATGAGGACGAACCTAAATCCTCCGACAAGACCC | |
| | AGCGTCCTCACCGTCCTGCACCAGGACTGGCTG | ACACCTGCCCCCCTTGCCCCGCTCCTGAAGCTGCTGGAGGCCCTTCAGTGTTTCTGTTTCCT | |
| | AATGGCAAGGAGTACAAGTGCAAGGTCTCCAAC | CCCAAGCCCAAGGACACACTGATGATTTCCAGAACCCCCGAGGTGACATGCGTGGTGGTGGA | |
| | AAAGCCCTCCCAGCCCCCATCGAGAAAACCATC | CGTGAGCCACGAAGATCCCGAGGTGAAGTTTAATTGGTACGTGGACGGCGTGGAAGTGCACA | |
| | TCCAAAGCCAAAGGGCAGCCCCGAGAACCACAG | ACGCCAAGACCAAACCCCGCGAGGAGCAGTATAATAGCACCTACAGAGTGGTGAGCGTGCTG | |
| | GTGTACACCCTGCCCCCATCCCGGGAGGAGATG | ACCGTGCTGCATCAGGACTGGCTGAACGGCAAGGAATACAAATGCAAGGTGAGCAACAAGGC | |
| | ACCAAGAACCAGGTCAGCCTGAGCTGCGCCGTC | CCTGCCTGCCCCTATCGAGAAGACCATCAGCAAAGCCAAGGGACAGCCAAGAGAACCCCAGG | |
| | AAAGGCTTCTATCCCAGCGACATCGCCGTGGAG | TGTACACCCTGCCCCCCAGCAGAGAAGAAATGACCAAGAACCAGGTGAGCCTGTGGTGCCTG | |
| | TGGGAGAGCAATGGGCAGCCGGAGAACAACTAC | GTGAAAGGCTTCTACCCCAGCGACATCGCCGTGGAGTGGGAGAGCAACGGCCAGCCAGAGAA | |
| | AAGACCACGCCTCCCGTGCTGGACTCCGACGGC | CAACTACAAGACAACACCCCCCGTGCTGGACAGCGACGGCTCTTTCTTCCTGTACAGTAAAC | |
| | TCCTTCTTCCTCGTGAGCAAGCTCACCGTGGAC | TGACCGTGGACAAATCAAGGTGGCAGCAGGGCAACGTGTTCAGCTGCTCCGTGATGCACGAG | |
| | AAGAGCAGGTGGCAGCAGGGGAACGTCTTCTCA | GCCCTGCACAACCACTATACCCAGAAGAGCCTGTCCCTGAGCCCCGGCAAG (SEQ ID | |
| | TGCTCCGTGATGCATGAGGCTCTGCACAACCGG | NO: 314) | |
| | TTCACGCAGAAGAGCCTCTCCCTGTCTCCGGGT | | |
| | AAA (SEQ ID NO: 295) | | |
| FUSE-874 | (SEQ ID NO: 114) | TCCTACTTCGGCAAGCTCGAATCCAAGCTCTCCGTCATCCGCAACCTCAACGACCAGGTCCT | |
| | | CTTTATCGACCAGGGCAACCGCCCTCTCTTCGAAGATATGACCGACTCCGACGTCCGCGACA | |
| | | ACGCTCCTAGGACCATTTTCATCATCTCCATGTACAAGGACTCCCAGCCCCGTGGAATGGCT | |
| | | GTTACAATTTCCGTCAAGGTGGAGAAAATCTCCACCCTGTCCGTGGAGAACAAGATCATTTC | |
| | | TTTCAAGGAGATGAACCCCCCCGACAACATCAAGGACACCAAGAGCGACATCATCTTCTTCC | |
| | | AGCGCTCCGTGCCCGGACATGATAACAAAATGCAGTTCGAGTCCTCCTCCTACGAGGGCTAC | |
| | | TTCCTCGCAGTCGAAAAGGAAAGGGACCTGTTCAAGTTGATCCTGAAGAAGGAGGACGAGCT | |
| | | CGGTGACAGGTCCATCATGTTCACCGTCCAGAACGAGGACGAGCCTAAATCCTCCGACAAAA | |
| | | CTCACACATGCCCACCGTGCCCAGCACCTGAACTCCTGGGGGGACCGTCAGTCTTCCTCTTC | |
| | | CCCCCAAAACCCAAGGACACCCTCATGATCTCCCGGACCCCCGAGGTCACATGCGTGGTGGT | |
| | | GGACGTGAGCCACGAAGACCCTGAGGTCAAGTTCAACTGGTACGTGGACGGCGTGGAGGTGC | |
| | | ATAATGCCAAGACAAAGCCGCGGGAGGAGCAGTACAACAGCACGTACCGTGTGGTCAGCGTC | |
| | | CTCACCGTCCTGCACCAGGACTGGCTGAATGGCAAGGAGTACAAGTGCAAGGTCTCCAACAA | |
| | | AGCCCTCCCAGCCCCCATCGAGAAAACCATCTCCAAAGCCAAAGGGCAGCCCCGAGAACCAC | |
| | | AGGTGTACACCCTGCCCCCATCCCGGGAGGAGATGACCAAGAACCAGGTCAGCCTGTGGTGC | |
| | | CTGGTCAAAGGCTTCTATCCCAGCGACATCGCCGTGGAGTGGGAGAGCAATGGGCAGCCGGA | |
| | | GAACAACTACAAGACCACGCCTCCCGTGCTGGACTCCGACGGCTCCTTCTTCCTCTACAGCA | |
| | | AGCTCACCGTGGACAAGAGCAGGTGGCAGCAGGGGAACGTCTTCTCATGCTCCGTGATGCAT | |
| | | GAGGCTCTGCACAACCACTACACGCAGAAGAGCCTCTCCCTGTCTCCGGGTAAA (SEQ ID | |
| | | NO: 177) | |
| FUSE-875 | (SEQ ID NO: 114) | GGAGGAGGAGGAAGTTACTTCGGCAAGCTGGAGTCCAAGCTGTCCGTGATCCGGAACCTGAA | |
| | | CGACCAGGTGCTGTTCATCGACCAGGGCAACAGGCCTCTCTTCGAGGATATGACCGACTCCG | |
| | | ACGTGAGGGATAACGCTCCAAGGACAATCTTCATCATCTCCATGTACAAGGACTCCCAGCCC | |
| | | AGGGGAATGGCTGTTACAATTTCCGTGAAAGTGGAGAAAATCTCCACCCTGTCCGTGGAAAA | |
| | | CAAGATCATCAGCTTCAAAGAGATGAACCCCCCCGACAACATCAAAGACACCAAATCCGACA | |
| | | TCATCTTCTTCCAGCGCTCTGTGCCCGGCCATGATAACAAAATGCAGTTCGAAAGCTCCTCC | |
| | | TACGAAGGCTACTTCCTGGCCGTCGAAAAGGAACGCGACCTCTTCAAGCTCATCCTGAAGAA | |
| | | GGAGGACGAACTGGGGGACAGGAGCATCATGTTCACCGTTCAGAACGAAGACGAGCCTAAAT | |
| | | CCTCCGACAAAACTCACACATGCCCACCGTGCCCAGCACCTGAACTCCTGGGGGGACCGTCA | |
| | | GTCTTCCTCTTCCCCCCAAAACCCAAGGACACCCTCATGATCTCCCGGACCCCCGAGGTCAC | |
| | | ATGCGTGGTGGTGGACGTGAGCCACGAAGACCCTGAGGTCAAGTTCAACTGGTACGTGGACG | |
| | | GCGTGGAGGTGCATAATGCCAAGACAAAGCCGCGGGAGGAGCAGTACAACAGCACGTACCGT | |
| | | GTGGTCAGCGTCCTCACCGTCCTGCACCAGGACTGGCTGAATGGCAAGGAGTACAAGTGCAA | |
| | | GGTCTCCAACAAAGCCCTCCCAGCCCCCATCGAGAAAACCATCTCCAAAGCCAAAGGGCAGC | |
| | | CCCGAGAACCACAGGTGTACACCCTGCCCCCATCCCGGGAGGAGATGACCAAGAACCAGGTC | |
| | | AGCCTGTGGTGCCTGGTCAAAGGCTTCTATCCCAGCGACATCGCCGTGGAGTGGGAGAGCAA | |
| | | TGGGCAGCCGGAGAACAACTACAAGACCACGCCTCCCGTGCTGGACTCCGACGGCTCCTTCT | |
| | | TCCTCTACAGCAAGCTCACCGTGGACAAGAGCAGGTGGCAGCAGGGGAACGTCTTCTCATGC | |
| | | TCCGTGATGCATGAGGCTCTGCACAACCACTACACGCAGAAGAGCCTCTCCCTGTCTCCGGG | |
| | | TAAA (SEQ ID NO: 178) | |
| FUSE-876 | (SEQ ID NO: 114) | GGTGGAGGAGGTTCTGGAGGTGGTGGATCATACTTCGGCAAGCTGGAGTCCAAGCTGTCCGT | |
| | | GATCCGGAACCTGAACGACCAGGTGCTGTTCATCGACCAGGGCAACAGGCCTCTCTTCGAGG | |
| | | ATATGACCGACTCCGACGTGAGGGATAACGCTCCAAGGACAATCTTCATCATCTCCATGTAC | |
| | | AAGGACTCCCAGCCCAGGGGAATGGCTGTTACAATTTCCGTGAAAGTGGAGAAAATCTCCAC | |
| | | CCTGTCCGTGGAAAACAAGATCATCAGCTTCAAAGAGATGAACCCCCCCGACAACATCAAAG | |
| | | ACACCAAATCCGACATCATCTTCTTCCAGCGCTCTGTGCCCGGCCATGATAACAAAATGCAG | |
| | | TTCGAAAGCTCCTCCTACGAAGGCTACTTCCTGGCCGTCGAAAAGGAACGCGACCTCTTCAA | |
| | | GCTCATCCTGAAGAAGGAGGACGAACTGGGGGACAGGAGCATCATGTTCACCGTTCAGAACG | |
| | | AAGACGAGCCTAAATCCTCCGACAAAACTCACACATGCCCACCGTGCCCAGCACCTGAACTC | |
| | | CTGGGGGGACCGTCAGTCTTCCTCTTCCCCCCAAAACCCAAGGACACCCTCATGATCTCCCG | |
| | | GACCCCCGAGGTCACATGCGTGGTGGTGGACGTGAGCCACGAAGACCCTGAGGTCAAGTTCA | |
| | | ACTGGTACGTGGACGGCGTGGAGGTGCATAATGCCAAGACAAAGCCGCGGGAGGAGCAGTAC | |
| | | AACAGCACGTACCGTGTGGTCAGCGTCCTCACCGTCCTGCACCAGGACTGGCTGAATGGCAA | |
| | | GGAGTACAAGTGCAAGGTCTCCAACAAAGCCCTCCCAGCCCCCATCGAGAAAACCATCTCCA | |
| | | AAGCCAAAGGGCAGCCCCGAGAACCACAGGTGTACACCCTGCCCCCATCCCGGGAGGAGATG | |
| | | ACCAAGAACCAGGTCAGCCTGTGGTGCCTGGTCAAAGGCTTCTATCCCAGCGACATCGCCGT | |
| | | GGAGTGGGAGAGCAATGGGCAGCCGGAGAACAACTACAAGACCACGCCTCCCGTGCTGGACT | |
| | | CCGACGGCTCCTTCTTCCTCTACAGCAAGCTCACCGTGGACAAGAGCAGGTGGCAGCAGGGG | |
| | | AACGTCTTCTCATGCTCCGTGATGCATGAGGCTCTGCACAACCACTACACGCAGAAGAGCCT | |
| | | CTCCCTGTCTCCGGGTAAA (SEQ ID NO: 179) | |
| FUSE-882 | GAAGTGCAGCTGGTGGAGAGCGGAGGAGGATTG | GAAGTGCAGCTGGTGGAGAGCGGAGGAGGATTGGTGCAGCCTGGAGGAAGCCTGAGGCTGAG | SEQ |
| | GTGCAGCCTGGAGGAAGCCTGAGGCTGAGTTGT | TTGTGCCGCTAGTGGGTTTACCTTTTCCGACTCCTGGATTCATTGGGTGAGGCAGGCCCCCG | ID |
| | GCCGCTAGTGGGTTTACCTTTTCCGACTCCTGG | GAAAGGGATTGGAATGGGTGGCATGGATTTCCCCCTATGGGGGAAGCACCTACTACGCTGAT | NO: 311 |
| | ATTCATTGGGTGAGGCAGGCCCCCGGAAAGGGA | TCCGTGAAGGGCAGGTTTACAATCTCCGCCGACACCTCCAAGAACACCGCCTACCTGCAGAT | |
| | TTGGAATGGGTGGCATGGATTTCCCCCTATGGG | GAGCTCCCTGAGGGCTGAGGACACCGCTGTTTACTACTGCGCCCGCCGACATTGGCCCGGAG | |
| | GGAAGCACCTACTACGCTGATTCCGTGAAGGGC | GATTCGATTACTGGGGCCAGGGAACCCTCGTGACCGTTTCTTCCGCTAGCACCAAGGGCCCA | |
| | AGGTTTACAATCTCCGCCGACACCTCCAAGAAC | TCGGTCTTCCCCCTGGCACCCTCCTCCAAGAGCACCTCTGGGGGCACAGCGGCCCTGGGCTG | |
| | ACCGCCTACCTGCAGATGAGCTCCCTGAGGGCT | CCTGGTCAAGGACTACTTCCCCGAACCGGTGACGGTGTCGTGGAACTCAGGCGCCCTGACCA | |
| | GAGGACACCGCTGTTTACTACTGCGCCCGCCGA | GCGGCGTGCACACCTTCCCGGCTGTCCTACAGTCCTCAGGACTCTACTCCCTCAGCAGCGTG | |
| | CATTGGCCCGGAGGATTCGATTACTGGGGCCAG | GTGACCGTGCCCTCCAGCAGCTTGGGCACCCAGACCTACATCTGCAACGTGAATCACAAGCC | |
| | GGAACCCTCGTGACCGTTTCTTCCGCTAGCACC | CAGCAACACCAAGGTGGACAAGAAAGTTGAGCCCAAATCTTGTGACAAAACTCACACATGCC | |
| | AAGGGCCCATCGGTCTTCCCCCTGGCACCCTCC | CACCGTGCCCAGCACCTGAACTCCTGGGGGGACCGTCAGTCTTCCTCTTCCCCCCAAAACCC | |
| | TCCAAGAGCACCTCTGGGGGCACAGCGGCCCTG | AAGGACACCCTCATGATCTCCCGGACCCCCGAGGTCACATGCGTGGTGGTGGACGTGAGCCA | |
| | GGCTGCCTGGTCAAGGACTACTTCCCCGAACCG | CGAAGACCCTGAGGTCAAGTTCAACTGGTACGTGGACGGCGTGGAGGTGCATAATGCCAAGA | |
| | GTGACGGTGTCGTGGAACTCAGGCGCCCTGACC | CAAAGCCGCGGGAGGAGCAGTACAACAGCACGTACCGTGTGGTCAGCGTCCTCACCGTCCTG | |
| | AGCGGCGTGCACACCTTCCCGGCTGTCCTACAG | CACCAGGACTGGCTGAATGGCAAGGAGTACAAGTGCAAGGTCTCCAACAAAGCCCTCCCAGC | |
| | TCCTCAGGACTCTACTCCCTCAGCAGCGTGGTG | CCCCATCGAGAAAACCATCTCCAAAGCCAAAGGGCAGCCCCGAGAACCACAGGTGTACACCC | |
| | ACCGTGCCCTCCAGCAGCTTGGGCACCCAGACC | TGCCCCCATCCCGGGAGGAGATGACCAAGAACCAGGTCAGCCTGTGGTGCCTGGTCAAAGGC | |
| | TACATCTGCAACGTGAATCACAAGCCCAGCAAC | TTCTATCCCAGCGACATCGCCGTGGAGTGGGAGAGCAATGGGCAGCCGGAGAACAACTACAA | |
| | ACCAAGGTGGACAAGAAAGTTGAGCCCAAATCT | GACCACGCCTCCCGTGCTGGACTCCGACGGCTCCTTCTTCCTCTACAGCAAGCTCACCGTGG | |
| | TGTGACAAAACTCACACATGCCCACCGTGCCCA | ACAAGAGCAGGTGGCAGCAGGGGAACGTCTTCTCATGCTCCGTGATGCATGAGGCTCTGCAC | |
| | GCACCTGAACTCCTGGGGGGACCGTCAGTCTTC | AACCACTACACGCAGAAGAGCCTCTCCCTGTCTCCGGGTGGTGGAGGAGGTTCTGGAGGTGG | |
| | CTCTTCCCCCCAAAACCCAAGGACACCCTCATG | TGGATCAGCTGCTGAACCTGTGGAAGATAATGTGATCAATTTTGTGGCCATGAAATTTATTG | |
| | ATCTCCCGGACCCCCGAGGTCACATGCGTGGTG | ACAACACCCTGTATTTCATCGCCGAAAACGATGAAAACGGCGGAGGATCTTATTTTGGAAAG | |
| | GTGGACGTGAGCCACGAAGACCCTGAGGTCAAG | CTGGAATCCAAATTGAGCGTGATCAGGAACCTGAACGATCAGGTGCTGTTTATCGACCAGGG | |
| | TTCAACTGGTACGTGGACGGCGTGGAGGTGCAT | CAACAGGCCCCTGTTCGAGGATATGACCGACAGTGACGTGAGGGACAACGCTCCCCGAACCA | |
| | AATGCCAAGACAAAGCCGCGGGAGGAGCAGTAC | TCTTCATCATCAGTAAGTATGGAGACTCCCAGCCAAGGGGACTGGCCGTTACAATTAGCGTG | |
| | AACAGCACGTACCGTGTGGTCAGCGTCCTCACC | AAAGTCGAGAAGATTTCCACATTGAGCGTGGAGAACAAGATCATCTCCTTCAAGGAGATGAA | |
| | GTCCTGCACCAGGACTGGCTGAATGGCAAGGAG | CCCCCCCGACAACATTAAGGACACCAAAAGCGATATCATCTTCTTCCAGAGGTCCGTGCCTG | |
| | TACAAGTGCAAGGTCTCCAACAAAGCCCTCCCA | GGCACGACAATAAAGTGCAGTTCGAGTCCAGCTCCTACGAGGGCTACTTTCTGGCAGTGGAG | |
| | GCCCCCATCGAGAAAACCATCTCCAAAGCCAAA | AAGGAAAGGGACCTGTTCAAGCTGATCCTGAAGAAGGAGGACGAGCTGGGGGACAGGTCCAT | |
| | GGGCAGCCCCGAGAACCACAGGTGTACACCCTG | CATGTTCACCGTCCAGAATGAGGAC (SEQ ID NO: 310) | |
| | CCCCCATCCCGGGAGGAGATGACCAAGAACCAG | | |
| | GTCAGCCTGAGCTGCGCCGTCAAAGGCTTCTAT | | |
| | CCCAGCGACATCGCCGTGGAGTGGGAGAGCAAT | | |
| | GGGCAGCCGGAGAACAACTACAAGACCACGCCT | | |
| | CCCGTGCTGGACTCCGACGGCTCCTTCTTCCTC | | |
| | GTGAGCAAGCTCACCGTGGACAAGAGCAGGTGG | | |
| | CAGCAGGGGAACGTCTTCTCATGCTCCGTGATG | | |
| | CATGAGGCTCTGCACAACAGATTCACGCAGAAG | | |
| | AGCCTCTCCCTGTCTCCGGGTAAA (SEQ ID | | |
| | NO: 296) | | |
| FUSE-887 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATACTATTAATTTCGTGGCCATGAAA | |
| | | TTCATCGATAATACTCTGTACTTCATCGCCGAAGATGATGAAAATCTGGAAAGTGATTACTT | |
| | | TGGAAAGCTGGAAAGCAAACTGAGCGTGATCCGCAACCTCAACGATCAGGTGCTGTTCATCG | |
| | | ACCAGGGCAATAGGCCCCTGTTCGAGGACATGACCGACTCTGACTCCAGGGACAACGCCCCT | |
| | | CGAACCATTTTCATCATCTCCATGTACAAGGACTCCCAGCCAAGGGGCATGGCCGTTACAAT | |
| | | TTCCGTGAAGTCCGAGAAAATCTCCACCCTGAGCAGCGAAAACAAAATCATCTCCTTCAAAG | |
| | | AGATGAACCCCCCCGACAACATCAAGGACACCAAATCCGACATCATCTTCTTCCAGAGGTCC | |
| | | GTGCCCGGCCACGATAACAAAATGCAGTTCGAGAGCAGCTCCTACGAGGGCTACTTCCTGGC | |
| | | CTCCGAAAAGGAAAGGGACCTGTTCAAGCTGATCCTGAAGAAGGAGGACGAGCTGGGGGACA | |
| | | GAAGCATCATGTTCACCGTGCAGAACGAGGAC (SEQ ID NO: 180) | |
| FUSE-888 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATCAGATTAATTTTGTGGCCATGAAA | |
| | | TTCATTGACAATACCCTGTATTTCATCGCCGAAGATGATGAAAACCTGGAATCTGATTACTT | |
| | | TGGAAAACTGGAAAGCAAACTGAGCGTGATCAGGAACCTGAACGATCAGGTGCTGTTTATCG | |
| | | ACCAGGGCAACAGGCCCCTCTTTGAGGACATGACCGACTCTGACTCTAGGGACAACGCCCCC | |
| | | AGGACCATCTTCATCATCTCCATGTACAAGGACTCCCAGCCAAGGGGAATGGCCGTGACAAT | |
| | | TTCCGTGAAATCCGAGAAGATCTCCACCCTGTCCTCCGAGAACAAAATCATCTCCTTCAAAG | |
| | | AGATGAACCCCCCCGACAACATCAAAGACACAAAATCCGACATCATCTTCTTCCAGAGGTCC | |
| | | GTGCCCGGTCACGACAACAAAATGCAGTTCGAGAGCAGCAGCTACGAGGGCTACTTCCTCGC | |
| | | CTCCGAAAAGGAAAGGGACCTGTTCAAGCTGATCCTGAAGAAGGAGGACGAGCTGGGGGACA | |
| | | GGTCTATCATGTTCACCGTCCAGAACGAGGAC (SEQ ID NO: 181) | |
| FUSE-889 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATGATATTAATTTCGTGGCCATGAAA | |
| | | TTCATCGATAATACTCTGTACTTCATCGCCGAAGATGATGAAAACCTGGAATCCGATTATTT | |
| | | TGGAAAACTGGAATCCAAACTGAGCGTGATCAGGAACCTCAACGACCAGGTGCTGTTCATTG | |
| | | ACCAGGGCAACAGGCCCCTGTTCGAGGATATGACAGACTCCGACGCCCGCGACAACGCTCCT | |
| | | AGAACAATCTTCATCATCTCTATGTACAAGGACTCCCAGCCCAGGGGAATGGCCGTTACAAT | |
| | | TTCCGTGAAGGCCGAGAAGATCTCCACCCTGTCCGCCGAAAACAAGATCATCTCCTTCAAAG | |
| | | AGATGAACCCCCCCGACAACATCAAGGACACCAAATCCGACATCATCTTCTTCCAGCGCTCC | |
| | | GTGCCCGGCCATGATAACAAAATGCAGTTCGAAAGCTCCTCCTACGAGGGATACTTTCTGGC | |
| | | CGCCGAAAAGGAAAGGGACCTGTTCAAGCTGATCCTGAAGAAGGAGGACGAGCTGGGGGACA | |
| | | GATCCATCATGTTCACCGTTCAGAACGAGGAC (SEQ ID NO: 182) | |
| FUSE-890 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATTTCATCAATTTTGTGGCCATGAAA | |
| | | TTCATTGATAACACCCTGTATTTCATCGCCGAAGATGATGAAAACCTGGAGTCTGATTACTT | |
| | | TGGAAAGCTGGAAAGCAAACTGAGCGTGATCCGCAACCTCAACGATCAGGTGCTGTTTATCG | |
| | | ACCAGGGCAACAGGCCACTGTTCGAGGACATGACCGACAGCGACGCCAGGGATAACGCCCCT | |
| | | AGAACAATCTTCATTATCTCCATGTACAAGGACTCCCAGCCCAGGGGAATGGCAGTGACAAT | |
| | | CTCCGTGAAGGCCGAGAAGATCAGCACCCTGAGCGCCGAAAACAAGATCATCTCCTTCAAGG | |
| | | AGATGAACCCCCCCGACAACATCAAGGACACCAAATCCGACATCATCTTCTTCCAGAGGTCC | |
| | | GTGCCCGGCCACGATAACAAAATGCAGTTTGAATCCTCCTCCTACGAAGGCTACTTTCTGGC | |
| | | CGCCGAAAAGGAAAGAGACTTGTTCAAGCTGATCCTGAAGAAGGAGGACGAGCTGGGGGACA | |
| | | GATCCATCATGTTCACCGTTCAGAACGAGGAC (SEQ ID NO: 183) | |
| FUSE-891 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATCTGATTAATTTTGTGGCCATGAAA | |
| | | TTCATCGATAACACCTTGTACTTTATCGCTGAAGATGATGAAAATCTGGAAAGTGACTACTT | |
| | | CGGAAAACTGGAAAGCAAATTGAGCGTGATCAGGAATCTGAACGATCAGGTGCTGTTCATCG | |
| | | ACCAGGGCAACCGCCCTCTGTTCGAGGATATGACCGACTCTGACGTGAGGGACAACGCCCCA | |
| | | AGGACAATCTTCATCATCTCTATGTACAAGGATTCCCAGCCCAGGGGCATGGCCGTTACAAT | |
| | | TTCCGTGAAGGTGGAGAAGATCTCCACACTGAGCGTGGAGAACAAGATCATCTCCTTCAAGG | |
| | | AGATGAACCCCCCCGACAACATCAAGGACACCAAATCCGACATCATCTTTTTCCAGAGGAGT | |
| | | GTGCCCGGTCACGACAACAAGATGCAGTTCGAATCCAGCTCCTACGAAGGCTACTTCCTCGC | |
| | | CGTCGAAAAGGAAAGGGACCTGTTCAAGCTGATCCTGAAGAAGGAGGACGAGTTGGGGGACA | |
| | | GGAGCATCATGTTCACCGTCCAGAACGAGGAC (SEQ ID NO: 184) | |
| FUSE-892 | (SEQ ID NO: 114) | GAACCTAAATCCTCCGACAAAACTCACACATGCCCACCGTGCCCAGCACCTGAACTCCTGGG | |
| | | GGGACCGTCAGTCTTCCTCTTCCCCCCAAAACCCAAGGACACCCTCATGATCTCCCGGACCC | |
| | | CCGAGGTCACATGCGTGGTGGTGGACGTGAGCCACGAAGACCCTGAGGTCAAGTTCAACTGG | |
| | | TACGTGGACGGCGTGGAGGTGCATAATGCCAAGACAAAGCCGCGGGAGGAGCAGTACAACAG | |
| | | CACGTACCGTGTGGTCAGCGTCCTCACCGTCCTGCACCAGGACTGGCTGAATGGCAAGGAGT | |
| | | ACAAGTGCAAGGTCTCCAACAAAGCCCTCCCAGCCCCCATCGAGAAAACCATCTCCAAAGCC | |
| | | AAAGGGCAGCCCCGAGAACCACAGGTGTACACCCTGCCCCCATCCCGGGAGGAGATGACCAA | |
| | | GAACCAGGTCAGCCTGTGGTGCCTGGTCAAAGGCTTCTATCCCAGCGACATCGCCGTGGAGT | |
| | | GGGAGAGCAATGGGCAGCCGGAGAACAACTACAAGACCACGCCTCCCGTGCTGGACTCCGAC | |
| | | GGCTCCTTCTTCCTCTACAGCAAGCTCACCGTGGACAAGAGCAGGTGGCAGCAGGGGAACGT | |
| | | CTTCTCATGCTCCGTGATGCATGAGGCTCTGCACAACCACTACACGCAGAAGAGCCTCTCCC | |
| | | TGTCTCCGGGTAAAGCTGCTGAACCTGTGGAAGATAATCATATTAATTTCGTGGCCATGAAA | |
| | | TTCATCGATAATACTCTGTACTTCATCGCCGAAGATGATGAAAATCTGGAAAGTGATTACTT | |
| | | TGGAAAGCTGGAAAGCAAACTGAGCGTGATCAGGAATCTCAACGATCAGGTGCTGTTCATCG | |
| | | ACCAGGGCAATAGGCCCCTGTTCGAGGACATGACCGACTCCGACGTGCGGGATAACGCCCCT | |
| | | AGAACAATCTTCATCATCTCCATGTACAAGGACTCCCAGCCAAGGGGCATGGCAGTGACAAT | |
| | | CAGCGTGAAAGTGGAGAAGATCTCCACACTGTCCGTGGAGAACAAGATCATCTCCTTCAAGG | |
| | | AGATGAACCCCCCCGACAACATCAAGGACACAAAGAGCGATATCATTTTCTTCCAGAGGAGC | |
| | | GTGCCCGGGCACGATAACAAAATGCAGTTCGAATCCAGCTCCTACGAAGGCTACTTCCTCGC | |
| | | CGTCGAAAAGGAAAGGGACCTGTTCAAGTTGATCCTGAAGAAGGAGGACGAATTGGGGGACA | |
| | | GGTCCATCATGTTCACCGTCCAGAACGAGGAC (SEQ ID NO: 185) | |
| FUSE-895 | (SEQ ID NO: 296) | GCTGCTGAACCTGTGGAAGATAATGTGATCAATTTTGTGGCCATGAAATTTATTGACAACAC | SEQ |
| | | CCTGTATTTCATCGCCGAAAACGATGAAAACGGCGGAGGATCTTATTTTGGAAAGCTGGAAT | ID |
| | | CCAAATTGAGCGTGATCAGGAACCTGAACGATCAGGTGCTGTTTATCGACCAGGGCAACAGG | NO: 311 |
| | | CCCCTGTTCGAGGATATGACCGACAGTGACGTGAGGGACAACGCTCCCCGAACCATCTTCAT | |
| | | CATCAGTAAGTATGGAGACTCCCAGCCAAGGGGACTGGCCGTTACAATTAGCGTGAAAGTCG | |
| | | AGAAGATTTCCACATTGAGCGTGGAGAACAAGATCATCTCCTTCAAGGAGATGAACCCCCCC | |
| | | GACAACATTAAGGACACCAAAAGCGATATCATCTTCTTCCAGAGGTCCGTGCCTGGGCACGA | |
| | | CAATAAAGTGCAGTTCGAGTCCAGCTCCTACGAGGGCTACTTTCTGGCAGTGGAGAAGGAAA | |
| | | GGGACCTGTTCAAGCTGATCCTGAAGAAGGAGGACGAGCTGGGGGACAGGTCCATCATGTTC | |
| | | ACCGTCCAGAATGAGGACGGTGGAGGAGGTTCTGGAGGTGGTGGATCAGACAAAACTCACAC | |
| | | ATGCCCACCGTGCCCAGCACCTGAACTCCTGGGGGGACCGTCAGTCTTCCTCTTCCCCCCAA | |
| | | AACCCAAGGACACCCTCATGATCTCCCGGACCCCCGAGGTCACATGCGTGGTGGTGGACGTG | |
| | | AGCCACGAAGACCCTGAGGTCAAGTTCAACTGGTACGTGGACGGCGTGGAGGTGCATAATGC | |
| | | CAAGACAAAGCCGCGGGAGGAGCAGTACAACAGCACGTACCGTGTGGTCAGCGTCCTCACCG | |
| | | TCCTGCACCAGGACTGGCTGAATGGCAAGGAGTACAAGTGCAAGGTCTCCAACAAAGCCCTC | |
| | | CCAGCCCCCATCGAGAAAACCATCTCCAAAGCCAAAGGGCAGCCCCGAGAACCACAGGTGTA | |
| | | CACCCTGCCCCCATCCCGGGAGGAGATGACCAAGAACCAGGTCAGCCTGTGGTGCCTGGTCA | |
| | | AAGGCTTCTATCCCAGCGACATCGCCGTGGAGTGGGAGAGCAATGGGCAGCCGGAGAACAAC | |
| | | TACAAGACCACGCCTCCCGTGCTGGACTCCGACGGCTCCTTCTTCCTCTACAGCAAGCTCAC | |
| | | CGTGGACAAGAGCAGGTGGCAGCAGGGGAACGTCTTCTCATGCTCCGTGATGCATGAGGCTC | |
| | | TGCACAACCACTACACGCAGAAGAGCCTCTCCCTGTCTCCGGGTAAA (SEQ ID | |
| | | NO: 315) | |
| FUSE-952 | (SEQ ID NO: 295) | GAGGTTCAGTTGCAGGAGAGCGGACCAGGACTGGTTAAGCCAAGCCAGAGCCTGAGCTTGAC | SEQ |
| | | ATGTAGCGTGACAGGATACAGCATTACAAGCAGCTATAGGTGGAACTGGATTAGGAAGTTCC | ID |
| | | CCGGGAACAGGTTGGAGTGGATGGGATATATCAACAGCGCAGGAATTTCCAACTACAACCCC | NO: 312 |
| | | AGCCTCAAGAGGAGGATCAGCATCACCCGGGACACCTCAAAGAACCAGTTCTTCCTGCAGGT | |
| | | CAACAGCGTGACCACCGAGGATGCTGCAACCTACTACTGCGCCCGATCCGATAACATGGGCA | |
| | | CCACACCATTCACCTACTGGGGGCAGGGAACCTTGGTGACAGTCTCTTCCGCTAGCACCAAG | |
| | | GGCCCATCGGTCTTCCCCCTGGCACCCTCCTCCAAGAGCACCTCTGGGGGCACAGCGGCCCT | |
| | | GGGCTGCCTGGTCAAGGACTACTTCCCCGAACCGGTGACGGTGTCGTGGAACTCAGGCGCCC | |
| | | TGACCAGCGGCGTGCACACCTTCCCGGCTGTCCTACAGTCCTCAGGACTCTACTCCCTCAGC | |
| | | AGCGTGGTGACCGTGCCCTCCAGCAGCTTGGGCACCCAGACCTACATCTGCAACGTGAATCA | |
| | | CAAGCCCAGCAACACCAAGGTGGACAAGAAAGTTGAGCCCAAATCTTGTGACAAAACTCACA | |
| | | CATGCCCACCGTGCCCAGCACCTGAAGCCGCAGGGGGACCGTCAGTCTTCCTCTTCCCCCCA | |
| | | AAACCCAAGGACACCCTCATGATCTCCCGGACCCCCGAGGTCACATGCGTGGTGGTGGACGT | |
| | | GAGCCACGAAGACCCTGAGGTCAAGTTCAACTGGTACGTGGACGGCGTGGAGGTGCATAATG | |
| | | CCAAGACAAAGCCGCGGGAGGAGCAGTACAACAGCACGTACCGTGTGGTCAGCGTCCTCACC | |
| | | GTCCTGCACCAGGACTGGCTGAATGGCAAGGAGTACAAGTGCAAGGTCTCCAACAAAGCCCT | |
| | | CCCAGCCCCCATCGAGAAAACCATCTCCAAAGCCAAAGGGCAGCCCCGAGAACCACAGGTGT | |
| | | ACACCCTGCCCCCATCCCGGGAGGAGATGACCAAGAACCAGGTCAGCCTGTGGTGCCTGGTC | |
| | | AAAGGCTTCTATCCCAGCGACATCGCCGTGGAGTGGGAGAGCAATGGGCAGCCGGAGAACAA | |
| | | CTACAAGACCACGCCTCCCGTGCTGGACTCCGACGGCTCCTTCTTCCTCTACAGCAAGCTCA | |
| | | CCGTGGACAAGAGCAGGTGGCAGCAGGGGAACGTCTTCTCATGCTCCGTGATGCATGAGGCT | |
| | | CTGCACAACCACTACACGCAGAAGAGCCTCTCCCTGTCTCCGGGTAAA (SEQ ID | |
| | | NO: 316) | |
| FUSE-1015 | GCTGCTGAACCTGTGGAAGATAATGTGATCAAT | (SEQ ID NO: 316) | SEQ |
| | TTCGTGGCCATGAAATTCATCGATAACACCCTG | | ID |
| | TACTTCATCGCTGAAAATGATGAAAACGGAGGA | | NO: 312 |
| | GGATCTTACTTTGGAAAGCTGGAATCCAAACTG | | |
| | AGCGTGATCAGGAACCTCAACGACCAGGTGCTG | | |
| | TTCATCGACCAGGGCAACAGGCCACTCTTCGAG | | |
| | GATATGACAGACAGCGACGTGAGGGACAATGCA | | |
| | CCCAGAACCATCTTCATCATCTCCAAGTACGGC | | |
| | GACAGCCAGCCCAGAGGACTGGCTGTGACAATC | | |
| | TCCGTGAAAGTGGAGAAGATTTCCACACTGAGC | | |
| | GTGGAGAACAAGATCATCTCCTTCAAGGAGATG | | |
| | AACCCCCCCGACAACATTAAAGACACCAAATCC | | |
| | GACATCATCTTCTTCCAGAGGTCCGTGCCCGGC | | |
| | CATGATAACAAAGTGCAGTTCGAGTCCAGCTCC | | |
| | TACGAGGGATACTTTCTGGCCGTCGAAAAGGAG | | |
| | AGGGACCTGTTCAAGCTCATTCTCAAGAAGGAG | | |
| | GACGAGCTGGGGGACAGATCCATTATGTTCACC | | |
| | GTGCAGAATGAGGACGAACCTAAGAGCTCCGAC | | |
| | AAGACACACACCTGCCCCCCCTGCCCCGCACCT | | |
| | GAGGCTGCTGGAGGACCTAGTGTGTTCCTGTTC | | |
| | CCCCCCAAACCCAAGGACACCCTGATGATTAGC | | |
| | AGAACCCCCGAGGTGACCTGCGTGGTCGTGGAC | | |
| | GTCTCCCACGAAGACCCCGAAGTGAAATTTAAC | | |
| | TGGTACGTGGACGGCGTCGAGGTGCACAACGCA | | |
| | AAGACCAAGCCCAGAGAAGAGCAATATAATAGC | | |
| | ACCTATAGAGTGGTGAGCGTGCTCACCGTGCTG | | |
| | CACCAGGACTGGCTGAACGGGAAGGAGTATAAG | | |
| | TGCAAGGTGAGCAACAAGGCACTGCCTGCCCCC | | |
| | ATAGAAAAAACCATCAGCAAGGCCAAGGGCCAG | | |
| | CCCAGAGAGCCCCAGGTGTACACCCTGCCCCCC | | |
| | AGCAGAGAGGAGATGACCAAGAACCAGGTGAGC | | |
| | CTGAGCTGCGCCGTGAAGGGCTTCTACCCCAGC | | |
| | GACATCGCCGTGGAGTGGGAGAGCAACGGCCAG | | |
| | CCCGAGAACAACTACAAGACCACCCCCCCCGTG | | |
| | CTGGACAGCGACGGCAGCTTCTTCCTGGTGAGC | | |
| | AAGCTGACCGTGGACAAGAGCAGATGGCAGCAG | | |
| | GGCAACGTGTTCAGCTGCAGCGTGATGCACGAG | | |
| | GCCCTGCACAACAGATTCACCCAGAAGAGCCTG | | |
| | AGCCTGAGCCCCGGCAAG (SEQ ID | | |
| | NO: 297) | | |
| FUSE-1017 | (SEQ ID NO: 131) | SEQ ID NO: 314 | SEQ |
| | | | ID |
| | | | NO: 188 |
| FUSE-1041 | (SEQ ID NO: 295) | SEQ ID NO: 169 | SEQ |
| | | | ID |
| | | | NO: 188 |
| FUSE-1042 | (SEQ ID NO: 297) | SEQ ID NO: 169 | SEQ |
| | | | ID |
| | | | NO: 188 |
| FUSE-1044 | GCTGCTGAACCTGTGGAAGATAATGTGATCAAT | SEQ ID NO: 169 | SEQ |
| | TTCGTGGCCATGAAGTTCATTGACAACACCCTG | | ID |
| | TACTTCATCGCTGAAAACGATGAGAACGGCGGA | | NO: 188 |
| | GGATCTTACTTCGGAAAACTGAAATCCAAGCTG | | |
| | AGCGTGATCAGGAACCTGAACGATCAGGTGCTG | | |
| | TTCATCGACCAGGGCAATAGGCCCCTGTTCGAG | | |
| | GATATGACAGACTCCGACGTGAGGGACAACGCT | | |
| | CCAAGAACCATCTTCATCATCTCTCACTACGGC | | |
| | GACTCCCAGCCAAGGGGAATGGCTGTGACAATC | | |
| | TCCGTGAAGGTGGAGAAGATTTCCACCCTGTCC | | |
| | GTTGAGAACAAGATCATCTCCTTCAAAGAGATG | | |
| | AACCCCCCCGACAACATCAAAGACACCAAATCC | | |
| | GACATCATCTTCTTCCAGCGCAGCGTGCCTGGG | | |
| | CATGATAACAAAATGCAGTTCGAAAGCTCCTCC | | |
| | TACGAGGGATACTTCCTCGCCGTCGAAAAGGAA | | |
| | AGGGACCTGTTCAAGCTGATCCTGAAGAAGGAG | | |
| | GATGAATTGGGGGATAGGAGCATCATGTTCACC | | |
| | GTCCAGAACGAAGACGAGCCTAAATCCTCCGAC | | |
| | AAGACACACACCTGCCCCCCCTGCCCCGCACCT | | |
| | GAGGCTGCTGGAGGACCTAGTGTGTTCCTGTTC | | |
| | CCCCCCAAACCCAAGGACACCCTGATGATTAGC | | |
| | AGAACCCCCGAGGTGACCTGCGTGGTCGTGGAC | | |
| | GTCTCCCACGAAGACCCCGAAGTGAAATTTAAC | | |
| | TGGTACGTGGACGGCGTCGAGGTGCACAACGCA | | |
| | AAGACCAAGCCCAGAGAAGAGCAATATAATAGC | | |
| | ACCTATAGAGTGGTGAGCGTGCTCACCGTGCTG | | |
| | CACCAGGACTGGCTGAACGGGAAGGAGTATAAG | | |
| | TGCAAGGTGAGCAACAAGGCACTGCCTGCCCCC | | |
| | ATAGAAAAAACCATCAGCAAGGCCAAGGGCCAG | | |
| | CCCAGAGAGCCCCAGGTGTACACCCTGCCCCCC | | |
| | AGCAGAGAGGAGATGACCAAGAACCAGGTGAGC | | |
| | CTGAGCTGCGCCGTGAAGGGCTTCTACCCCAGC | | |
| | GACATCGCCGTGGAGTGGGAGAGCAACGGCCAG | | |
| | CCCGAGAACAACTACAAGACCACCCCCCCCGTG | | |
| | CTGGACAGCGACGGCAGCTTCTTCCTGGTGAGC | | |
| | AAGCTGACCGTGGACAAGAGCAGATGGCAGCAG | | |
| | GGCAACGTGTTCAGCTGCAGCGTGATGCACGAG | | |
| | GCCCTGCACAACAGGTTCACCCAGAAGAGCCTG | | |
| | AGCCTGAGCCCCGGCAAG (SEQ ID | | |
| | NO: 298) | | |
| FUSE-1109 | TACTTCGGCAAGCTGCAGTCCAAGCTGTCCGTG | SEQ ID NO: 169 | SEQ |
| | ATCAGGAATCTCAACGACCAGGTGTTGTTCATC | | ID |
| | GACCAGGGGAACAGGCCCCTCTTCGAGGATATG | | NO: 188 |
| | ACCGACTCCGACGTGAGGGATAACGCTCCCCGT | | |
| | ACAATCTTCATCATCTCCATGTACGACGACTCC | | |
| | CAGCCCAGGGGTAAGGCTGTTACCATTAGCGTG | | |
| | AAAGTGGAGAAAATTTCCACACTGAGCGTGGAG | | |
| | AACAAGATCATCTCCTTTAAGGAGATGAACCCC | | |
| | CCCGACAACATTAAAGACACAAAGAGCGACATC | | |
| | ATCTTTTTCCAGAGGAGTGTGCCCGGCCACGAC | | |
| | AACAAGATGCAGTTCGAGTCCAGCTCCTACGAA | | |
| | GGATACTTCCTGGCCGTCGAAAAGGAAAGGGAC | | |
| | CTGTTCAAGTTGATCCTGAAGAAGGAGGATGAA | | |
| | TTGGGGGACAGGAGCATCATGTTCACCGTCCAG | | |
| | AACGAGGACGAACCTAAATCCTCCGACAAGACA | | |
| | CACACCTGCCCCCCCTGCCCCGCACCTGAGGCT | | |
| | GCTGGAGGACCTAGTGTGTTCCTGTTCCCCCCC | | |
| | AAACCCAAGGACACCCTGATGATTAGCAGAACC | | |
| | CCCGAGGTGACCTGCGTGGTCGTGGACGTCTCC | | |
| | CACGAAGACCCCGAAGTGAAATTTAACTGGTAC | | |
| | GTGGACGGCGTCGAGGTGCACAACGCAAAGACC | | |
| | AAGCCCAGAGAAGAGCAATATAATAGCACCTAT | | |
| | AGAGTGGTGAGCGTGCTCACCGTGCTGCACCAG | | |
| | GACTGGCTGAACGGGAAGGAGTATAAGTGCAAG | | |
| | GTGAGCAACAAGGCACTGCCTGCCCCCATAGAA | | |
| | AAAACCATCAGCAAGGCCAAGGGCCAGCCCAGA | | |
| | GAGCCCCAGGTGTACACCCTGCCCCCCAGCAGA | | |
| | GAGGAGATGACCAAGAACCAGGTGAGCCTGAGC | | |
| | TGCGCCGTGAAGGGCTTCTACCCCAGCGACATC | | |
| | GCCGTGGAGTGGGAGAGCAACGGCCAGCCCGAG | | |
| | AACAACTACAAGACCACCCCCCCCGTGCTGGAC | | |
| | AGCGACGGCAGCTTCTTCCTGGTGAGCAAGCTG | | |
| | ACCGTGGACAAGAGCAGATGGCAGCAGGGCAAC | | |
| | GTGTTCAGCTGCAGCGTGATGCACGAGGCCCTG | | |
| | CACAACAGATTCACCCAGAAGAGCCTGTCCCTG | | |
| | TCCCCCGGA (SEQ ID NO: 299) | | |
| FUSE-1110 | TACTTCGGCAAGCTGCAGTCCAAGCTGTCCGTG | SEQ ID NO: 169 | SEQ |
| | ATCAGGAATCTCAACGACCAGGTGTTGAAGATC | | ID |
| | GACCAGGGGAACAGGCCCCTCTTCGAGGATATG | | NO: 188 |
| | ACCGACTCCGACGTGAGGGATAACGCTCCCCGT | | |
| | ACAATCTTCATTATCTCCATGTACGACGACTCC | | |
| | CAGCCCAGGGGTAAAGCCGTTACAATTTCCGTG | | |
| | AAGGTGGAGAAGATTTCCACATTGTCCGTGGAG | | |
| | AACAAGATCATCTCCTTCAAAGAGATGAACCCC | | |
| | CCCGACAACATCAAGGACACCAAAAGCGACATC | | |
| | ATCTTCTTCCAGCGCAGTGTGCCTGGGCACGAC | | |
| | AACAAAATGCAGTTCGAATCCAGCTCCTACGAG | | |
| | GGCTACTTTCTGGCCGTCGAAAAGGAAAGGGAC | | |
| | CTGTTCAAACTGATCCTGAAGAAGGAGGACGAG | | |
| | CTGGGCGACAGGTCCATCATGTTCACCGTCCAG | | |
| | AACGAGGACGAACCTAAATCCTCCGACAAGACA | | |
| | CACACCTGCCCCCCCTGCCCCGCACCTGAGGCT | | |
| | GCTGGAGGACCTAGTGTGTTCCTGTTCCCCCCC | | |
| | AAACCCAAGGACACCCTGATGATTAGCAGAACC | | |
| | CCCGAGGTGACCTGCGTGGTCGTGGACGTCTCC | | |
| | CACGAAGACCCCGAAGTGAAATTTAACTGGTAC | | |
| | GTGGACGGCGTCGAGGTGCACAACGCAAAGACC | | |
| | AAGCCCAGAGAAGAGCAATATAATAGCACCTAT | | |
| | AGAGTGGTGAGCGTGCTCACCGTGCTGCACCAG | | |
| | GACTGGCTGAACGGGAAGGAGTATAAGTGCAAG | | |
| | GTGAGCAACAAGGCACTGCCTGCCCCCATAGAA | | |
| | AAAACCATCAGCAAGGCCAAGGGCCAGCCCAGA | | |
| | GAGCCCCAGGTGTACACCCTGCCCCCCAGCAGA | | |
| | GAGGAGATGACCAAGAACCAGGTGAGCCTGAGC | | |
| | TGCGCCGTGAAGGGCTTCTACCCCAGCGACATC | | |
| | GCCGTGGAGTGGGAGAGCAACGGCCAGCCCGAG | | |
| | AACAACTACAAGACCACCCCCCCCGTGCTGGAC | | |
| | AGCGACGGCAGCTTCTTCCTGGTGAGCAAGCTG | | |
| | ACCGTGGACAAGAGCAGATGGCAGCAGGGCAAC | | |
| | GTGTTCAGCTGCAGCGTGATGCACGAGGCCCTG | | |
| | CACAACAGATTCACCCAGAAGAGCCTGTCCCTG | | |
| | TCCCCCGGA (SEQ ID NO: 300) | | |
| FUSE-1111 | TACTTCGGCAAGCTGCAGTCCAAGCTGTCCGTG | SEQ ID NO: 169 | SEQ |
| | ATCAGGAATCTCAACGACCAGGTGTTGTTCATC | | ID |
| | GACCAGGGGAACAGGCCCCTCTTCGAGGATATG | | NO: 188 |
| | ACCGACTCCGACGTGAGGGATAACGCTCCCCGT | | |
| | ACAATCTTCATCATCTCCATGTACGACGACTCC | | |
| | CAGCCCAGGGGTAAGGCTGTTACCATTAGCGTG | | |
| | AAAGTGGAGAAAATTTCCACACTGAGCGTGGAG | | |
| | AACAAGATCATCTCCTTTAAGGAGATGAACCCC | | |
| | CCCGACAACATTAAAGACACAAAGAGCGACATC | | |
| | ATCTTTTTCCAGAGGAGTGTGCCCGGCCACGAC | | |
| | AACGAAATGCAGTTCGAGTCCAGCTCCTACGAA | | |
| | GGATACTTCCTGGCCGTCGAAAAGGAAAGGGAC | | |
| | CTCTTCAAGCTGATCCTGAAGAAGGAGGACGAG | | |
| | TTGGGGGACAGAAGCATCATGTTTACCGTGCAG | | |
| | AACGAGGACGAACCTAAATCCTCCGACAAGACA | | |
| | CACACCTGCCCCCCCTGCCCCGCACCTGAGGCT | | |
| | GCTGGAGGACCTAGTGTGTTCCTGTTCCCCCCC | | |
| | AAACCCAAGGACACCCTGATGATTAGCAGAACC | | |
| | CCCGAGGTGACCTGCGTGGTCGTGGACGTCTCC | | |
| | CACGAAGACCCCGAAGTGAAATTTAACTGGTAC | | |
| | GTGGACGGCGTCGAGGTGCACAACGCAAAGACC | | |
| | AAGCCCAGAGAAGAGCAATATAATAGCACCTAT | | |
| | AGAGTGGTGAGCGTGCTCACCGTGCTGCACCAG | | |
| | GACTGGCTGAACGGGAAGGAGTATAAGTGCAAG | | |
| | GTGAGCAACAAGGCACTGCCTGCCCCCATAGAA | | |
| | AAAACCATCAGCAAGGCCAAGGGCCAGCCCAGA | | |
| | GAGCCCCAGGTGTACACCCTGCCCCCCAGCAGA | | |
| | GAGGAGATGACCAAGAACCAGGTGAGCCTGAGC | | |
| | TGCGCCGTGAAGGGCTTCTACCCCAGCGACATC | | |
| | GCCGTGGAGTGGGAGAGCAACGGCCAGCCCGAG | | |
| | AACAACTACAAGACCACCCCCCCCGTGCTGGAC | | |
| | AGCGACGGCAGCTTCTTCCTGGTGAGCAAGCTG | | |
| | ACCGTGGACAAGAGCAGATGGCAGCAGGGCAAC | | |
| | GTGTTCAGCTGCAGCGTGATGCACGAGGCCCTG | | |
| | CACAACAGATTCACCCAGAAGAGCCTGTCCCTG | | |
| | TCCCCCGGA (SEQ ID NO: 301) | | |
| FUSE-1112 | TACTTCGGCAAGCTGAAGTCCAAGCTGTCCGTG | SEQ ID NO: 169 | SEQ |
| | ATCAGGAATCTGAACGACCAGGTGCTGTTTATC | | ID |
| | GACCAGGGCAACAGGCCCCTGTTCGAGGATATG | | NO: 188 |
| | ACCGACTCCGACGTGAGGGACAACGCACCAAGG | | |
| | ACCATCTTCATCATCTCCATGTACGACGACTCC | | |
| | CAGCCAAGGGGCAGGGCTGTTACAATCTCCGTG | | |
| | AAGGTGGAGAAGATTTCCACACTGAGCGTGGAA | | |
| | AACAAGATCATCTCCTTCAAAGAGATGAACCCC | | |
| | CCCGACAACATCAAGGACACCAAATCCGACATC | | |
| | ATCTTCTTCCAGCGCAGCGTGCCCGGCCATGAT | | |
| | AACAAGATGCAGTTCGAAAGCTCCTCCTACGAG | | |
| | GGATACTTTCTGGCCGTCGAAAAGGAAAGGGAC | | |
| | CTGTTCAAGCTGATCCTGAAGAAGGAGGACGAG | | |
| | TTGGGGGACAGAAGCATCATGTTCACCGTCCAG | | |
| | AACGAAGACGAACCTAAATCCTCCGACAAGACA | | |
| | CACACCTGCCCCCCCTGCCCCGCACCTGAGGCT | | |
| | GCTGGAGGACCTAGTGTGTTCCTGTTCCCCCCC | | |
| | AAACCCAAGGACACCCTGATGATTAGCAGAACC | | |
| | CCCGAGGTGACCTGCGTGGTCGTGGACGTCTCC | | |
| | CACGAAGACCCCGAAGTGAAATTTAACTGGTAC | | |
| | GTGGACGGCGTCGAGGTGCACAACGCAAAGACC | | |
| | AAGCCCAGAGAAGAGCAATATAATAGCACCTAT | | |
| | AGAGTGGTGAGCGTGCTCACCGTGCTGCACCAG | | |
| | GACTGGCTGAACGGGAAGGAGTATAAGTGCAAG | | |
| | GTGAGCAACAAGGCACTGCCTGCCCCCATAGAA | | |
| | AAAACCATCAGCAAGGCCAAGGGCCAGCCCAGA | | |
| | GAGCCCCAGGTGTACACCCTGCCCCCCAGCAGA | | |
| | GAGGAGATGACCAAGAACCAGGTGAGCCTGAGC | | |
| | TGCGCCGTGAAGGGCTTCTACCCCAGCGACATC | | |
| | GCCGTGGAGTGGGAGAGCAACGGCCAGCCCGAG | | |
| | AACAACTACAAGACCACCCCCCCCGTGCTGGAC | | |
| | AGCGACGGCAGCTTCTTCCTGGTGAGCAAGCTG | | |
| | ACCGTGGACAAGAGCAGATGGCAGCAGGGCAAC | | |
| | GTGTTCAGCTGCAGCGTGATGCACGAGGCCCTG | | |
| | CACAACAGATTCACCCAGAAGAGCCTGTCCCTG | | |
| | TCCCCCGGA (SEQ ID NO: 302) | | |
| FUSE-1113 | GGAGGAGGAGGAAGTTACTTTGGAAAGCTGGAG | SEQ ID NO: 316 | SEQ |
| | AGCAAGCTGAGTGTGATCAGGAACCTGAATGAC | | ID |
| | CAGGTGTTGTTTATTGATCAGGGGAATAGGCCC | | NO: 312 |
| | TTGTTTGAGGATATGACCGACAGCGATGTGAGG | | |
| | GACAACGCCCCAAGGACAATCTTCATCATCTCC | | |
| | AAGTACGGCGATAGCCAGCCCAGGGGTTTGGCA | | |
| | GTTACCATCAGCGTGAAGGTGGAGAAGATTAGC | | |
| | ACACTGAGCGTGGAGAACAAGATCATCTCCTTC | | |
| | AAGGAGATGAACCCCCCTGACAACATCAAGGAC | | |
| | ACCAAGAGCGATATTATCTTTTTCCAGAGGAGT | | |
| | GTGCCCGGCCACGACAACAAAGTGCAGTTCGAA | | |
| | AGCTCCTCCTACGAGGGCTACTTCCTGGCCGTC | | |
| | GAGAAGGAGAGGGACCTGTTCAAGCTGATCCTG | | |
| | AAGAAGGAGGACGAGTTGGGGGACAGGAGCATC | | |
| | ATGTTCACCGTCCAGAACGAAGACGAACCTAAA | | |
| | TCCTCCGACAAGACACACACCTGCCCCCCCTGC | | |
| | CCCGCACCTGAGGCTGCTGGAGGACCTAGTGTG | | |
| | TTCCTGTTCCCCCCCAAACCCAAGGACACCCTG | | |
| | ATGATTAGCAGAACCCCCGAGGTGACCTGCGTG | | |
| | GTCGTGGACGTCTCCCACGAAGACCCCGAAGTG | | |
| | AAATTTAACTGGTACGTGGACGGCGTCGAGGTG | | |
| | CACAACGCAAAGACCAAGCCCAGAGAAGAGCAA | | |
| | TATAATAGCACCTATAGAGTGGTGAGCGTGCTC | | |
| | ACCGTGCTGCACCAGGACTGGCTGAACGGGAAG | | |
| | GAGTATAAGTGCAAGGTGAGCAACAAGGCACTG | | |
| | CCTGCCCCCATAGAAAAAACCATCAGCAAGGCC | | |
| | AAGGGCCAGCCCAGAGAGCCCCAGGTGTACACC | | |
| | CTGCCCCCCAGCAGAGAGGAGATGACCAAGAAC | | |
| | CAGGTGAGCCTGAGCTGCGCCGTGAAGGGCTTC | | |
| | TACCCCAGCGACATCGCCGTGGAGTGGGAGAGC | | |
| | AACGGCCAGCCCGAGAACAACTACAAGACCACC | | |
| | CCCCCCGTGCTGGACAGCGACGGCAGCTTCTTC | | |
| | CTGGTGAGCAAGCTGACCGTGGACAAGAGCAGA | | |
| | TGGCAGCAGGGCAACGTGTTCAGCTGCAGCGTG | | |
| | ATGCACGAGGCCCTGCACAACAGATTCACCCAG | | |
| | AAGAGCCTGAGCCTGAGCCCCGGCAAG (SEQ | | |
| | ID NO: 303) | | |
| FUSE-1116 | TACTTTGGAAAGCTGGAGAGCAAGCTGAGTGTG | SEQ ID NO: 169 | SEQ |
| | ATCAGGAACCTGAATGACCAGGTGTTGTTTATT | | ID |
| | GATCAGGGGAATAGGCCCTTGTTTGAGGATATG | | NO: 188 |
| | ACCGACAGCGATGTGAGGGACAACGCCCCAAGG | | |
| | ACAATCTTCATCATCTCCAAGTACGGCGATAGC | | |
| | CAGCCCAGGGGTTTGGCAGTTACCATCAGCGTG | | |
| | AAGGTGGAGAAGATTAGCACACTGAGCGTGGAG | | |
| | AACAAGATCATCTCCTTCAAGGAGATGAACCCC | | |
| | CCTGACAACATCAAGGACACCAAGAGCGATATT | | |
| | ATCTTTTTCCAGAGGAGTGTGCCCGGCCACCGT | | |
| | AACAAAGTGCAGTTCGAAAGCTCCTCCTACGAG | | |
| | GGCTACTTCCTGGCCGTCGAGAAGGAGAGGGAC | | |
| | CTGTTCAAGCTGATCCTGAAGAAGGAGGACGAG | | |
| | TTGGGGGACAGGAGCATCATGTTCACCGTCCAG | | |
| | AACGAAGACGAACCTAAATCCTCCGACAAGACA | | |
| | CACACCTGCCCCCCCTGCCCCGCACCTGAGGCT | | |
| | GCTGGAGGACCTAGTGTGTTCCTGTTCCCCCCC | | |
| | AAACCCAAGGACACCCTGATGATTAGCAGAACC | | |
| | CCCGAGGTGACCTGCGTGGTCGTGGACGTCTCC | | |
| | CACGAAGACCCCGAAGTGAAATTTAACTGGTAC | | |
| | GTGGACGGCGTCGAGGTGCACAACGCAAAGACC | | |
| | AAGCCCAGAGAAGAGCAATATAATAGCACCTAT | | |
| | AGAGTGGTGAGCGTGCTCACCGTGCTGCACCAG | | |
| | GACTGGCTGAACGGGAAGGAGTATAAGTGCAAG | | |
| | GTGAGCAACAAGGCACTGCCTGCCCCCATAGAA | | |
| | AAAACCATCAGCAAGGCCAAGGGCCAGCCCAGA | | |
| | GAGCCCCAGGTGTACACCCTGCCCCCCAGCAGA | | |
| | GAGGAGATGACCAAGAACCAGGTGAGCCTGAGC | | |
| | TGCGCCGTGAAGGGCTTCTACCCCAGCGACATC | | |
| | GCCGTGGAGTGGGAGAGCAACGGCCAGCCCGAG | | |
| | AACAACTACAAGACCACCCCCCCCGTGCTGGAC | | |
| | AGCGACGGCAGCTTCTTCCTGGTGAGCAAGCTG | | |
| | ACCGTGGACAAGAGCAGATGGCAGCAGGGCAAC | | |
| | GTGTTCAGCTGCAGCGTGATGCACGAGGCCCTG | | |
| | CACAACAGATTCACCCAGAAGAGCCTGAGCCTG | | |
| | AGCCCCGGCAAG (SEQ ID NO: 304) | | |
| FUSE- | GAAGTGCAGCTGCAGGAGTCCGGTCCTGGATTG | | SEQ |
| | GTGAAGCCCAGCCAGAGCCTGTCCCTGACATGC | | ID |
| | TCTGTGACAGGATACTCCATCACAAGCTCCTAC | | NO: 313 |
| | AGGTGGAACTGGATTAGGAAGTTCCCCGGCAAC | | |
| | AGGCTGGAGTGGATGGGATACATCAACAGCGCC | | |
| | GGAATCTCCAACTACAACCCCTCCCTGAAAAGG | | |
| | CGCATCAGCATCACTAGGGACACCTCCAAGAAC | | |
| | CAGTTCTTCCTCCAGGTGAACAGCGTGACCACC | | |
| | GAGGACGCAGCAACATACTACTGCGCCAGGTCC | | |
| | GACAACATGGGCACAACCCCTTTCACATACTGG | | |
| | GGACAGGGCACCCTCGTGACCGTTTCTTCCGCT | | |
| | AGCACCAAGGGCCCATCGGTCTTCCCCCTGGCA | | |
| | CCCTCCTCCAAGAGCACCTCTGGGGGCACAGCG | | |
| | GCCCTGGGCTGCCTGGTCAAGGACTACTTCCCC | | |
| | GAACCGGTGACGGTGTCGTGGAACTCAGGCGCC | | |
| | CTGACCAGCGGCGTGCACACCTTCCCGGCTGTC | | |
| | CTACAGTCCTCAGGACTCTACTCCCTCAGCAGC | | |
| | GTGGTGACCGTGCCCTCCAGCAGCTTGGGCACC | | |
| | CAGACCTACATCTGCAACGTGAATCACAAGCCC | | |
| | AGCAACACCAAGGTGGACAAGAAAGTTGAGCCC | | |
| | AAATCTTGTGACAAAACTCACACATGCCCACCG | | |
| | TGCCCAGCACCTGAAGCCGCAGGGGGACCGTCA | | |
| | GTCTTCCTCTTCCCCCCAAAACCCAAGGACACC | | |
| | CTCATGATCTCCCGGACCCCCGAGGTCACATGC | | |
| | GTGGTGGTGGACGTGAGCCACGAAGACCCTGAG | | |
| | GTCAAGTTCAACTGGTACGTGGACGGCGTGGAG | | |
| | GTGCATAATGCCAAGACAAAGCCGCGGGAGGAG | | |
| | CAGTACAACAGCACGTACCGTGTGGTCAGCGTC | | |
| | CTCACCGTCCTGCACCAGGACTGGCTGAATGGC | | |
| | AAGGAGTACAAGTGCAAGGTCTCCAACAAAGCC | | |
| | CTCCCAGCCCCCATCGAGAAAACCATCTCCAAA | | |
| | GCCAAAGGGCAGCCCCGAGAACCACAGGTGTAC | | |
| | ACCCTGCCCCCATCCCGGGAGGAGATGACCAAG | | |
| | AACCAGGTCAGCCTGACCTGCCTGGTCAAAGGC | | |
| | TTCTATCCCAGCGACATCGCCGTGGAGTGGGAG | | |
| | AGCAATGGGCAGCCGGAGAACAACTACAAGACC | | |
| | ACGCCTCCCGTGCTGGACTCCGACGGCTCCTTC | | |
| | TTCCTCTACAGCAAGCTCACCGTGGACAAGAGC | | |
| | AGGTGGCAGCAGGGGAACGTCTTCTCATGCTCC | | |
| | GTGATGCATGAGGCTCTGCACAACCACTACACG | | |
| | CAGAAGAGCCTCTCCCTGTCTCCGGGTAAA | | |
| | (SEQ ID NO: 305) | | |
| FUSE-1177 | GGAGGAGGAGGAAGTTACTTCGGCAAGCTGCAG | SEQ ID NO: 169 | SEQ |
| | TCCAAGCTGTCCGTGATCAGGAATCTCAACGAC | | ID |
| | CAGGTGTTGTTCATCGACCAGGGGAACAGGCCC | | NO: 188 |
| | CTCTTCGAGGATATGACCGACTCCGACGTGAGG | | |
| | GATAACGCTCCCCGTACAATCTTCATCATCTCC | | |
| | ATGTACGACGACTCCCAGCCCAGGGGTAAGGCT | | |
| | GTTACCATTAGCGTGAAAGTGGAGAAAATTTCC | | |
| | ACACTGAGCGTGGAGAACAAGATCATCTCCTTT | | |
| | AAGGAGATGAACCCCCCCGACAACATTAAAGAC | | |
| | ACAAAGAGCGACATCATCTTTTTCCAGAGGAGT | | |
| | GTGCCCGGCCACGACAACAAGATGCAGTTCGAG | | |
| | TCCAGCTCCTACGAAGGATACTTCCTGGCCGTC | | |
| | GAAAAGGAAAGGGACCTGTTCAAGTTGATCCTG | | |
| | AAGAAGGAGGATGAATTGGGGGACAGGAGCATC | | |
| | ATGTTCACCGTCCAGAACGAGGACGAACCTAAA | | |
| | TCCTCCGACAAGACACACACCTGCCCCCCCTGC | | |
| | CCCGCACCTGAGGCTGCTGGAGGACCTAGTGTG | | |
| | TTCCTGTTCCCCCCCAAACCCAAGGACACCCTG | | |
| | ATGATTAGCAGAACCCCCGAGGTGACCTGCGTG | | |
| | GTCGTGGACGTCTCCCACGAAGACCCCGAAGTG | | |
| | AAATTTAACTGGTACGTGGACGGCGTCGAGGTG | | |
| | CACAACGCAAAGACCAAGCCCAGAGAAGAGCAA | | |
| | TATAATAGCACCTATAGAGTGGTGAGCGTGCTC | | |
| | ACCGTGCTGCACCAGGACTGGCTGAACGGGAAG | | |
| | GAGTATAAGTGCAAGGTGAGCAACAAGGCACTG | | |
| | CCTGCCCCCATAGAAAAAACCATCAGCAAGGCC | | |
| | AAGGGCCAGCCCAGAGAGCCCCAGGTGTACACC | | |
| | CTGCCCCCCAGCAGAGAGGAGATGACCAAGAAC | | |
| | CAGGTGAGCCTGAGCTGCGCCGTGAAGGGCTTC | | |
| | TACCCCAGCGACATCGCCGTGGAGTGGGAGAGC | | |
| | AACGGCCAGCCCGAGAACAACTACAAGACCACC | | |
| | CCCCCCGTGCTGGACAGCGACGGCAGCTTCTTC | | |
| | CTGGTGAGCAAGCTGACCGTGGACAAGAGCAGA | | |
| | TGGCAGCAGGGCAACGTGTTCAGCTGCAGCGTG | | |
| | ATGCACGAGGCCCTGCACAACAGATTCACCCAG | | |
| | AAGAGCCTGTCCCTGTCCCCCGGA (SEQ ID | | |
| | NO: 306) | | |

Polynucleotide 3 sequences
(SEQ ID NO: 186)
GACATTCTGCTGACCCAGAGCCCCGTGATCCTGAGCGTGAGCCCCGGAGAGAGAGTGAGCTTTAGCTGCAGAGC
CAGCCAGAGCATCGGCACCAACATCCACTGGTATCAGCAGCGGACCAACGGCAGCCCCAGACTGCTGATCAAGT
ACGCCAGTGAGAGCATCAGCGGAATCCCTTCTAGGTTCAGCGGATCAGGCAGCGGCACCGACTTCACCCTGAGT
ATCAACAGCGTGGAAAGCGAAGACATCGCCGACTATTACTGCCAGCAGAACAATAACTGGCCCACCACCTTCGG
CGCCGGCACCAAGCTGGAACTGAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGTTCGCCCGTCACAAAGAGCTTCAACAGGGGAGAGTGT
(SEQ ID NO: 187)
GACATCCAGATGACCCAGTCCCCATCCTCCCTGTCTGCTTCTGTGGGAGACAGGGTGACCATCACCTGCAGAGC
TTCCCAGGACGTGAACACAGCCGTGGCTTGGTACCAGCAGAAGCCTGGAAAAGCCCCCAAGCTGCTGATCTACT
CCGCCTCCTTTCTGTATTCCGGCGTGCCTTCCCGGTTTTCCGGAAGCAGGTCTGGTACAGACTTCACCCTGACC
ATCTCCTCCCTGCAGCCTGAAGATTTTGCTACCTACTACTGCCAGCAGCACTACACCACCCCTCCTACATTCGG
CCAGGGTACAAAGGTGGAAATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGTTCGCCCGTCACAAAGAGCTTCAACAGGGGAGAGTGT
(SEQ ID NO: 188)
GAAATCGTGCTGACCCAGTCCCCCGCTACCTTGAGTCTGTCCCCTGGAGAGAGAGCCACCCTGTCTTGTAGGGC
TAGTCAGTCCGTGAGCAGCTATCTGGCCTGGTATCAGCAGAAGCCCGGCCAGGCTCCTAGGTTGTTGATTTACG
ATGCCTCCAACAGAGCCACCGGAATCCCTGCACGGTTCTCCGGTTCCGGTTCTGGTACAGACTTCACCCTGACT
ATCAGCAGTCTGGAGCCCGAGGATTTCGCCGTTTACTACTGCCAGCAGTCCTCCAACTGGCCCAGGACATTCGG
TCAGGGCACCAAGGTGGAGATCAAGCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGTTCGCCCGTCACAAAGAGCTTCAACAGGGGAGAGTGT
(SEQ ID NO: 311)
GATATCCAGATGACCCAGTCCCCCTCCTCCCTGTCTGCTTCTGTGGGAGACAGAGTGACCATTACCTGTAGAGC
CTCCCAGGACGTGTCCACCGCTGTTGCTTGGTACCAGCAGAAGCCCGGCAAGGCTCCAAAACTGCTCATCTACT
CCGCCTCCTTTCTGTACTCCGGCGTGCCATCCCGCTTCTCAGGATCTGGATCTGGAACCGACTTCACCTTGACC
ATCTCCTCCCTGCAGCCCGAGGACTTCGCTACTTACTACTGCCAGCAGTACCTCTACCACCCCGCTACCTTCGG
CCAGGGAACAAAGGTTGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGTTCGCCCGTCACAAAGAGCTTCAACAGGGGAGAGTGT
(SEQ ID NO: 312)
GACATCGTGATGACCCAGGGAACCCTCCCAAACCCAGTGCCTTCTGGAGAATCCGTGAGCATTACCTGCAGGTC
CTCCAAATCCCTGCTGTACTCCGACGGAAAAACCTACCTGAACTGGTACCTGCAGAGGCCCGGCCAGTCCCCCC
AGCTGCTGATCTACTGGATGTCCACCAGGGCCTCCGGCGTGTCCGACAGGTTCTCCGGCTCCGGCTCCGGCACC
GACTTCACCCTGAAGATCTCCGGCGTGGAGGCCGAGGACGTGGGCATCTACTACTGCCAGCAGGGCCTGGAGTT
CCCCACCTTCGGCGGCGGCACCAAGCTGGAGCTGAAGCGTACGGTGGCTGCACCATCTGTCTTCATCTTCCCGC
CATCTGATGAGCAGTTGAAATCTGGAACTGCCTCTGTTGTGTGCCTGCTGAATAACTTCTATCCCAGAGAGGCC
AAAGTACAGTGGAAGGTGGATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGAAACACAAAGTCTACGCCT
GCGAAGTCACCCATCAGGGCCTGAGTTCGCCCGTCACAAAGAGCTTCAACAGGGGAGAGTGT
(SEQ ID NO: 313)
GGAGGAGGAGGAAGTTACTTTGGAAAGCTGGAGAGCAAGCTGAGTGTGATCAGGAACCTGAATGACCAGGTGTT
GTTTATTGATCAGGGGAATAGGCCCTTGTTTGAGGATATGACCGACAGCGATGTGAGGGACAACGCCCCAAGGA
CAATCTTCATCATCTCCAAGTACGGCGATAGCCAGCCCAGGGGTTTGGCAGTTACCATCAGCGTGAAGGTGGAG
AAGATTAGCACACTGAGCGTGGAGAACAAGATCATCTCCTTCAAGGAGATGAACCCCCCTGACAACATCAAGGA
CACCAAGAGCGATATTATCTTTTTCCAGAGGAGTGTGCCCGGCCACGACAACAAAGTGCAGTTCGAAAGCTCCT
CCTACGAGGGCTACTTCCTGGCCGTCGAGAAGGAGAGGGACCTGTTCAAGCTGATCCTGAAGAAGGAGGACGAG
TTGGGGGACAGGAGCATCATGTTCACCGTCCAGAACGAAGACGGTGGCGGAGGGTCTGGTGGTGGAGGATCCGG
GGGAGGAGGTTCAGGAGGCGGGGGAAGTGACATCGTGATGACCCAGGGAACCCTCCCAAACCCAGTGCCTTCTG
GAGAATCCGTGAGCATTACCTGCAGGTCCTCCAAATCCCTGCTGTACTCCGACGGAAAAACCTACCTGAACTGG
TACCTGCAGAGGCCCGGCCAGTCCCCCCAGCTGCTGATCTACTGGATGTCCACCAGGGCCTCCGGCGTGTCCGA
CAGGTTCTCCGGCTCCGGCTCCGGCACCGACTTCACCCTGAAGATCTCCGGCGTGGAGGCCGAGGACGTGGGCA
TCTACTACTGCCAGCAGGGCCTGGAGTTCCCCACCTTCGGCGGCGGCACCAAGCTGGAGCTGAAGCGTACGGTG
GCTGCACCATCTGTCTTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTCCAATCGGGTAACTCCC
AGGAGAGTGTCACAGAGCAGGACAGCAAGGACAGCACCTACAGCCTCAGCAGCACCCTGACGCTGAGCAAAGCA
GACTACGAGAAACACAAAGTCTACGCCTGCGAAGTCACCCATCAGGGCCTGAGTTCGCCCGTCACAAAGAGCTT
CAACAGGGGAGAGTGT

TABLE 3
Yields of exemplary fusion proteins.

| | Yield |
|---|---|
| FUSE-422 | 191 mg/L |
| FUSE-423 | 214 mg/L |
| FUSE-424 | 198 mg/L |
| FUSE-441 | 386 mg/L |
| FUSE-442 | 197 mg/L |
| FUSE-462 | 304 mg/L |
| FUSE-480 | 130 mg/L |
| FUSE-481 | 199 mg/L |
| FUSE-484 | 164 mg/L |
| FUSE-485 | 348 mg/L |
| FUSE-486 | 400 mg/L |
| FUSE-487 | 205 mg/L |
| FUSE-499 | 410 mg/L |
| FUSE-500 | 253 mg/L |
| FUSE-501 | 147 mg/L |
| FUSE-502 | 159 mg/L |
| FUSE-503 | 44 mg/L |
| FUSE-504 | 135 mg/L |
| FUSE-505 | 162 mg/L |
| FUSE-507 | 557 mg/L |
| FUSE-508 | 34 mg/L |
| FUSE-509 | 537 mg/L |
| FUSE-510 | 32 mg/L |
| FUSE-516 | 215 mg/L |
| FUSE-517 | 150 mg/L |
| FUSE-545 | 144 mg/L |
| FUSE-546 | 143 mg/L |
| FUSE-547 | 135 mg/L |
| FUSE-583 | 86 mg/L |
| FUSE-584 | 124 mg/L |
| FUSE-585 | 136 mg/L |
| FUSE-586 | 107 mg/L |
| FUSE-587 | 228 mg/L |
| FUSE-599 | 76 mg/L |
| FUSE-600 | 79 mg/L |
| FUSE-601 | 86 mg/L |
| FUSE-602 | 64 mg/L |
| FUSE-624 | 14 mg/L |
| FUSE-625 | 65 mg/L |
| FUSE-626 | 72 mg/L |
| FUSE-627 | 96 mg/L |
| FUSE-645 | 48 mg/L |
| FUSE-658 | 76 mg/L |
| FUSE-659 | 107 mg/L |
| FUSE-660 | 63 mg/L |
| FUSE-669 | 103 mg/L |
| FUSE-670 | 56 mg/L |
| FUSE-675 | 234 mg/L |
| FUSE-676 | 44 mg/L |
| FUSE-682 | 14 mg/L |
| FUSE-683 | 12 mg/L |
| FUSE-684 | 8 mg/L |
| FUSE-685 | 56 mg/L |
| FUSE-686 | 57 mg/L |
| FUSE-691 | 69 mg/L |
| FUSE-694 | 67 mg/L |
| FUSE-756 | 46 mg/L |
| FUSE-757 | 96 mg/L |
| FUSE-758 | 64 mg/L |
| FUSE-775 | 79 mg/L |
| FUSE-782 | 41 mg/L |
| FUSE-783 | 99 mg/L |
| FUSE-827 | 207 mg/L |
| FUSE-832 | 206 mg/L |
| FUSE-874 | 246 mg/L |
| FUSE-875 | 137 mg/L |
| FUSE-876 | 216 mg/L |
| FUSE-882 | 107 mg/L |
| FUSE-887 | 155 mg/L |
| FUSE-888 | 149 mg/L |
| FUSE-889 | 223 mg/L |
| FUSE-890 | 190 mg/L |
| FUSE-891 | 181 mg/L |
| FUSE-892 | 371 mg/L |
| FUSE-895 | 187 mg/L |
| FUSE-952 | 308 mg/L |
| FUSE-1015 | 395 mg/L |
| FUSE-1017 | 58 mg/L |
| FUSE-1041 | 234 mg/L |
| FUSE-1042 | 68 mg/L |
| FUSE-1044 | 121 mg/L |
| FUSE-1109 | 76 mg/L |
| FUSE-1110 | 54 mg/L |
| FUSE-1111 | 52 mg/L |
| FUSE-1112 | 76 mg/L |
| FUSE-1113 | 147 mg/L |
| FUSE-1116 | 62 mg/L |
| FUSE-1176 | 202 mg/L |
| FUSE-1177 | 133 mg/L |

TABLE 4
Amino acid sequences for Fc heterodimerization including different versions of
knobs-into-holes of respective polypeptide arms, forming (part of) the
protein capable of translocating to the ER, for exemplary fusion proteins.

| | First Polypeptide Arm | Second Polypeptide Arm |
|---|---|---|
| 7.8.60 | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTDNQVSLTCLVKGFYPS | APIEKTISKAKGQPRRPRVYTLPPSRDELTKNQVSLVCLVKGFYPS |
| | DIAVEWESNGQPENNYKTTPPVLMSDGSFFLASKLTVDKSRWQQGN | DIAVEWESNGQPENNYKTTPPVLDSDGSFFLYSVLTVDKSRWQQGN |
| | VFSCSVMHEALHNHYTQKSLSLSPGK (SEQ ID NO: 189) | VFSCSVMHEALHNHYTQKSLSLSPGK (SEQ ID NO: 190) |
| 7.8.60 | GQPREPQVYTLPPSRDELTDNQVSLTCLVKGFYPSDIAVEWESNGQ | GQPRRPRVYTLPPSRDELTKNQVSLVCLVKGFYPSDIAVEWESNGQ |
| CH3 | PENNYKTTPPVLMSDGSFFLASKLTVDKSRWQQGNVFSCSVMHEAL | PENNYKTTPPVLDSDGSFFLYSVLTVDKSRWQQGNVFSCSVMHEAL |
| | HNHYTQKSLSLSPGK (SEQ ID NO: 191) | HNHYTQKSLSLSPGK (SEQ ID NO: 192) |
| A107 | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTENQVSLTCLVKGFYPS | APIEKTISKAKGQPREPQVYTLPPSRDNLTKNQVSLTCLVKGFYPS |
| | DIAVEWESNGQPENNYKTTPPVLDSDGSFFLYSWLTVDKSRWQQGN | DIAVEWESNGQPENNYKTTPPVLVSDGSFTLYSKLTVDKSRWQQGN |
| | VFSCSVMHEALHNHYTQKSLSLSPGK (SEQ ID NO: 193) | VFSCSVMHEALHNHYTQKSLSLSPGK (SEQ ID NO: 194) |
| A107 | GQPREPQVYTLPPSRDELTENQVSLTCLVKGFYPSDIAVEWESNGQ | GQPREPQVYTLPPSRDNLTKNQVSLTCLVKGFYPSDIAVEWESNGQ |
| CH3 | PENNYKTTPPVLDSDGSFFLYSWLTVDKSRWQQGNVFSCSVMHEAL | PENNYKTTPPVLVSDGSFTLYSKLTVDKSRWQQGNVFSCSVMHEAL |
| | HNHYTQKSLSLSPGK (SEQ ID NO: 195) | HNHYTQKSLSLSPGK (SEQ ID NO: 196) |
| DD-KK | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTKNQVSLTCLVKGFYPS | APIEKTISKAKGQPREPQVYTLPPSRDKLTKNQVSLTCLVKGFYPS |
| | DIAVEWESNGQPENNYDTTPPVLDSDGSFFLYSDLTVDKSRWQQGN | DIAVEWESNGQPENNYKTTPPVLKSDGSFFLYSKLTVDKSRWQQGN |
| | VFSCSVMHEALHNHYTQKSLSLSPGK (SEQ ID NO: 197) | VFSCSVMHEALHNHYTQKSLSLSPGK (SEQ ID NO: 198) |
| DD-KK | GQPREPQVYTLPPSRDELTKNQVSLTCLVKGFYPSDIAVEWESNGQ | GQPREPQVYTLPPSRDKLTKNQVSLTCLVKGFYPSDIAVEWESNGQ |
| CH3 | PENNYDTTPPVLDSDGSFFLYSDLTVDKSRWQQGNVFSCSVMHEAL | PENNYKTTPPVLKSDGSFFLYSKLTVDKSRWQQGNVFSCSVMHEAL |
| | HNHYTQKSLSLSPGK (SEQ ID NO: 199) | HNHYTQKSLSLSPGK (SEQ ID NO: 200) |
| Duobody | PSVELFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTKNQVSLTCLVKGFYPS | APIEKTISKAKGQPREPQVYTLPPSRDELTKNQVSLTCLVKGFYPS |
| | DIAVEWESNGQPENNYKTTPPVLDSDGSFFLYSRLTVDKSRWQQGN | DIAVEWESNGQPENNYKTTPPVLDSDGSFLLYSKLTVDKSRWQQGN |
| | VESCSVMHEALHNHYTQKSLSLSPGK (SEQ ID NO: 201) | VESCSVMHEALHNHYTQKSLSLSPGK (SEQ ID NO: 202) |
| Duobody | GQPREPQVYTLPPSRDELTKNQVSLTCLVKGFYPSDIAVEWESNGQ | GQPREPQVYTLPPSRDELTKNQVSLTCLVKGFYPSDIAVEWESNGQ |
| CH3 | PENNYKTTPPVLDSDGSFFLYSRLTVDKSRWQQGNVFSCSVMHEAL | PENNYKTTPPVLDSDGSFLLYSKLTVDKSRWQQGNVFSCSVMHEAL |
| | HNHYTQKSLSLSPGK (SEQ ID NO: 203) | HNHYTQKSLSLSPGK (SEQ ID NO: 204) |
| Electro- | GQPREPQVYTLPPSRDELTKNQVSLTCLVKGFYPSDIAVEWESNGQ | GQPREPQVYTLPPSRDELTKNQVSLTCLVKGFYPSDIAVEWESNGQ |
| static | PENNYKTTPPVLDSDGSFFLYSNLTVDKSRWQQGNVFSCSVMHEAL | PENNYKTTPPVLKSDGSFFLYSKLTVDKSRWQQGNVESCSVMHEAL |
| Steering | HNHYTQKSLSLSPGK (SEQ ID NO: 205) | HNHYTQKSLSLSPGK (SEQ ID NO: 206) |
| CH3 | | |
| Electro- | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| static | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| Steering | APIEKTISKAKGQPREPQVYTLPPSRDELTKNQVSLTCLVKGFYPS | APIEKTISKAKGQPREPQVYTLPPSRDELTKNQVSLTCLVKGFYPS |
| | DIAVEWESNGQPENNYKTTPPVLDSDGSFFLYSNLTVDKSRWQQGN | DIAVEWESNGQPENNYKTTPPVLKSDGSFFLYSKLTVDKSRWQQGN |
| | VFSCSVMHEALHNHYTQKSLSLSPGK (SEQ ID NO: 207) | VFSCSVMHEALHNHYTQKSLSLSPGK (SEQ ID NO: 208) |
| EW-RVT | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTENQVSLTCLVKGFYPS | APIEKTISKAKGQPREPRVYTLPPSRDELTKNQVSLTCLVKGFYPS |
| | DIAVEWESNGQPENNYKTTPPVLDSDGSFFLYSWLTVDKSRWQQGN | DIAVEWESNGQPENNYKTTPPVLVSDGSFTLYSKLTVDKSRWQQGN |
| | VFSCSVMHEALHNHYTQKSLSLSPGK (SEQ ID NO: 193) | VFSCSVMHEALHNHYTQKSLSLSPGK (SEQ ID NO: 209) |
| EW-RVT | PSVELFPPKPKDTLMISRTPEVTCVVVDVSHEDPEVKENWYVDGVE | PSVFLFPPKPKDTLMISRTPEVTCVVVDVSHEDPEVKENWYVDGVE |
| (2) | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVCTLPPSRDELTENQVSLTCLVKGFYPS | APIEKTISKAKGQPREPRVYTLPPCRDELTKNQVSLTCLVKGFYPS |
| | DIAVEWESNGQPENNYKTTPPVLDSDGSFFLYSWLTVDKSRWQQGN | DIAVEWESNGQPENNYKTTPPVLVSDGSFTLYSKLTVDKSRWQQGN |
| | VFSCSVMHEALHNHYTQKSLSLSPGK (SEQ ID NO: 210) | VFSCSVMHEALHNHYTQKSLSLSPGK (SEQ ID NO: 211) |
| EW-RVT | GQPREPQVCTLPPSRDELTENQVSLTCLVKGFYPSDIAVEWESNGQ | GQPREPRVYTLPPCRDELTKNQVSLTCLVKGFYPSDIAVEWESNGQ |
| (2) CH3 | PENNYKTTPPVLDSDGSFFLYSWLTVDKSRWQQGNVFSCSVMHEAL | PENNYKTTPPVLVSDGSFTLYSKLTVDKSRWQQGNVFSCSVMHEAL |
| | HNHYTQKSLSLSPGK (SEQ ID NO: 212) | HNHYTQKSLSLSPGK (SEQ ID NO: 213) |
| EW-RVT | GQPREPQVYTLPPSRDELTENQVSLTCLVKGFYPSDIAVEWESNGQ | GQPREPRVYTLPPSRDELTKNQVSLTCLVKGFYPSDIAVEWESNGQ |
| CH3 | PENNYKTTPPVLDSDGSFFLYSWLTVDKSRWQQGNVFSCSVMHEAL | PENNYKTTPPVLVSDGSFTLYSKLTVDKSRWQQGNVFSCSVMHEAL |
| | HNHYTQKSLSLSPGK (SEQ ID NO: 195) | HNHYTQKSLSLSPGK (SEQ ID NO: 214) |
| HA-TF | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| (1) | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTKNQVHLTCLVKGFYPS | APIEKTISKAKGQPREPQVTTLPPSRDELTKNQVSLTCLVKGFYPS |
| | DIAVEWESNGQPENNYKTTPPVLDSDGSFALYSKLTVDKSRWQQGN | DIAVEWESNGQPENNYKTFPPVLDSDGSFFLYSKLTVDKSRWQQGN |
| | VFSCSVMHEALHNHYTQKSLSLSPGK (SEQ ID NO: 215) | VFSCSVMHEALHNHYTQKSLSLSPGK (SEQ ID NO: 216) |
| HA-TF | GQPREPQVYTLPPSRDELTKNQVHLTCLVKGFYPSDIAVEWESNGQ | GQPREPQVTTLPPSRDELTKNQVSLTCLVKGFYPSDIAVEWESNGQ |
| (1) CH3 | PENNYKTTPPVLDSDGSFALYSKLTVDKSRWQQGNVFSCSVMHEAL | PENNYKTTPPVLDSDGSFFLVSKLTVDKSRWQQGNVFSCSVMHEAL |
| | HNHYTQKSLSLSPGK (SEQ ID NO: 217) | HNHYTQKSLSLSPGK (SEQ ID NO: 218) |
| Knob- | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| in-hole | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| (1) | APIEKTISKAKGQPREPQVYTLPPSRDELTKNQVSLWCLVKGFYPS | APIEKTISKAKGQPREPQVYTLPPSRDELTKNQVSLSCAVKGFYPS |
| | DIAVEWESNGQPENNYKTTPPVLDSDGSFFLYSKLTVDKSRWQQGN | DIAVEWESNGQPENNYKTTPPVLDSDGSFFLVSKLTVDKSRWQQGN |
| | VFSCSVMHEALHNHYTQKSLSLSPGK (SEQ ID NO: 219) | VFSCSVMHEALHNHYTQKSLSLSPGK (SEQ ID NO: 220) |
| Knob- | GQPREPQVYTLPPSRDELTKNQVSLWCLVKGFYPSDIAVEWESNGQ | GQPREPQVYTLPPSRDELTKNQVSLSCAVKGFYPSDIAVEWESNGQ |
| in-hole | PENNYKTTPPVLDSDGSFFLYSKLTVDKSRWQQGNVFSCSVMHEAL | PENNYKTTPPVLDSDGSFFLVSKLTVDKSRWQQGNVFSCSVMHEAL |
| (1) CH3 | HNHYTQKSLSLSPGK (SEQ ID NO: 221) | HNHYTQKSLSLSPGK (SEQ ID NO: 222) |
| Knob- | PSVELFPPKPKDTLMISRTPEVTCVVVDVSHEDPEVKENWYVDGVE | PSVFLFPPKPKDTLMISRTPEVTCVVVDVSHEDPEVKENWYVDGVE |
| in-hole | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| (2) | APIEKTISKAKGQPREPQVYTLPPCRDELTKNQVSLWCLVKGFYPS | APIEKTISKAKGQPREPQVCTLPPSRDELTKNQVSLSCAVKGFYPS |
| | DIAVEWESNGQPENNYKTTPPVLDSDGSFFLYSKLTVDKSRWQQGN | DIAVEWESNGQPENNYKTTPPVLDSDGSFFLVSKLTVDKSRWQQGN |
| | VFSCSVMHEALHNHYTQKSLSLSPGK (SEQ ID NO: 223) | VFSCSVMHEALHNHYTQKSLSLSPGK (SEQ ID NO: 224) |
| Knob- | GQPREPQVYTLPPCRDELTKNQVSLWCLVKGFYPSDIAVEWESNGQ | GQPREPQVCTLPPSRDELTKNQVSLSCAVKGFYPSDIAVEWESNGQ |
| in-hole | PENNYKTTPPVLDSDGSFFLYSKLTVDKSRWQQGNVFSCSVMHEAL | PENNYKTTPPVLDSDGSFFLVSKLTVDKSRWQQGNVFSCSVMHEAL |
| (2) CH3 | HNHYTQKSLSLSPGK (SEQ ID NO: 225) | HNHYTQKSLSLSPGK (SEQ ID NO: 226) |
| XmAb | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYTLPPSRDELTKNQVSLTCDVSGFYPS | APIEKTISKAKGQPREPQVYTLPPSRDQLTKNQVKLTCLVKGFYPS |
| | DIAVEWESNGQPENNYKTTPPVLDSDGSFFLYSKLTVDKSRWQQGN | DIAVEWESNGQPENNYKTTPPVLDSDGSFFLYSKLTVDKSRWQQGN |
| | VESCSVMHEALHNHYTQKSLSLSPGK (SEQ ID NO: 227) | VFSCSVMHEALHNHYTQKSLSLSPGK (SEQ ID NO: 228) |
| XmAb | GQPREPQVYTLPPSRDELTKNQVSLTCDVSGFYPSDIAVEWESNGQ | GQPREPQVYTLPPSRDQLTKNQVKLTCLVKGFYPSDIAVEWESNGQ |
| CH3 | PENNYKTTPPVLDSDGSFFLYSKLTVDKSRWQQGNVFSCSVMHEAL | PENNYKTTPPVLDSDGSFFLYSKLTVDKSRWQQGNVFSCSVMHEAL |
| | HNHYTQKSLSLSPGK (SEQ ID NO: 229) | HNHYTQKSLSLSPGK (SEQ ID NO: 230) |
| ZW1 | PSVFLFPPKPKDTLMISRTPEVTCVVVDVSHEDPEVKENWYVDGVE | PSVFLFPPKPKDTLMISRTPEVTCVVVDVSHEDPEVKENWYVDGVE |
| | VHNAKTKPREEQYNSTYRVVSVLTVLHQDWLNGKEYKCKVSNKALP | VHNAKTKPREEQYNSTYRVVSVLTVLHQDWLNGKEYKCKVSNKALP |
| | APIEKTISKAKGQPREPQVYVYPPSRDELTKNQVSLTCLVKGFYPS | APIEKTISKAKGQPREPQVYVLPPSRDELTKNQVSLLCLVKGFYPS |
| | DIAVEWESNGQPENNYKTTPPVLDSDGSFALVSKLTVDKSRWQQGN | DIAVEWESNGQPENNYLTWPPVLDSDGSFFLYSKLTVDKSRWQQGN |
| | VFSCSVMHEALHNHYTQKSLSLSPGK (SEQ ID NO: 231) | VFSCSVMHEALHNHYTQKSLSLSPGK (SEQ ID NO: 232) |
| ZW1 CH3 | GQPREPQVYVYPPSRDELTKNQVSLTCLVKGFYPSDIAVEWESNGQ | GQPREPQVYVLPPSRDELTKNQVSLLCLVKGFYPSDIAVEWESNGQ |
| | PENNYKTTPPVLDSDGSFALVSKLTVDKSRWQQGNVFSCSVMHEAL | PENNYLTWPPVLDSDGSFFLYSKLTVDKSRWQQGNVFSCSVMHEAL |
| | HNHYTQKSLSLSPGK (SEQ ID NO: 233) | HNHYTQKSLSLSPGK (SEQ ID NO: 234) |

TABLE 5
The sequences, antibodies and fragments thereof (e.g., VHH, Fab regions, or scFv) in the references, publications, patent documents,
webpages contained in this table are hereby incorporated by reference as though fully set forth.

| Name | Alternative names | Description |
|---|---|---|
| Anti-CD3 | | |
| muromonab | OKT3 (Ortho | First monoclonal murine antibody to become available for therapy in humans-mouse IgG2a is directed against |
| | Kung T3) | the CD3 epsilon chain of the CD3/TCR complex |
| Teplizumab | hOKT3γ1 | CDRs granted onto human IgG backbone with two mutations (L234/A, L235A) |
| | (Ala-Ala) | |
| | and MGA031 | |
| Otelixizumab | ChAglyCD3, | chimeric and humanized IgG1 bears a single mutation in the γl Fc portion (N297/A) to avoid glycosylation and |
| | TRX4, | thus inhibit FcR binding |
| | GSK2136525 | |
| Visilizumab | Nuvion, HuM291 | humanized IgG2 antibody rendered non mitogenic by two point mutations in its Fc region (V234/A, V237/A) |
| Foralumab | 28F11-AE; | entirely human anti-CD3 mAb whose Fc portion of this human IgG1 was mutated (L234/A, V235E) such that |
| | NI-0401 | the mAb is non FcR binding in vitro and exhibits only minor cytokine release |
| Bispecific | | |
| Blinatumomab | AMG 103 | linked scFv(s) againt CD19 on tumor cells and CD3 on T cells; no Fc domain; Anti-CD3 comes from murine |
| | | mAb: L2K-07; published sequences encoding scFv addressing CD3ε (diL2K, the de-immunized version of the |
| | | mouse monoclonal antibody L2K) |
| flotetuzumab | MGD006 | DART targeting CD3 and CD123 |
| Cibisatamab | | 1 binding site for CD3e chaim and 2 CEA binding sties for cancer cells |
| Catumaxomab | | consists of mouse IgG2 and rat IgG2b and targets CD3 on T cells and EpCAM on tumor cells |
| Mosunetuzumab | | CD20 × CD3 T-cell engaging bsAb |

All candidates shared the same CD3-specific singlechain antibody derived from mouse monoclonal antibody L2K, which is distinct from monoclonal
antibodies TR66 and OKT3. L2K was subjected to a procedure that identifies human T helper cell epitopes and removes by point mutations MHC
class II anchor amino acids within potential epitopes (Jones et al., 2004). This resulted in “de-immunized” L2K (diL2K)
(sequence encoding scFv addressing CD3e is disclosed in K. Brischwein et al./Molecular Immunology 43 (2006) 1129-1143)

| TR66 | monoclonal anti-human CD3 epsilon antibody |
|---|---|
| PIT565 | Trispecific: CD19 × CD3 × CD2 |
| Four CD3 mAb-based binders: | |
| OKT3, L2K, h38E4.v1 and TRX4 | |
| OKT3 | |
| TRX4 | |
| h38E4.v1 | Binds N-terminal portion of CD3e extracellular domain |
| L2K | L2K, for instance shares over 90% sequence identity with OKT3 and recognizes the same epitope on CD3ε but |
| | differs in affinity by about 100-fold |
| Anti-CD16 | |
| 3G8 | monoclonal anti-human CD16 |
| U.S. Pat. No. | |
| 9,035,026 | |
| HRS-3/A9 | A9 is anti-CD16 mouse IgG1/λ Mab |
| Humanization of anti-CD16 Fab | Protein Engineering, Design and Selection, Volume 22, Issue 3, March 2009, Pages 175-188. the CDRs of the |
| | anti-CD16 mouse IgG1/λ MAb A9 were grafted onto human Ig sequences (CDRs were transplanted onto a |
| | human κ light chain + Affinity maturation via radon mutation in the humanized VH and VK domains) |
| AFM13 | Tetravalent bispecific CD30/CD16 (binds CD16A on NK cell surface, thus activating and recruiting NK cells to |
| | CD30+ tumor cell) |
| GTB-3550 | Trispecific CD16/IL-15/CD33 (anti-CD16 nanobody, or Ist generation uses scFv anti-CD16) |
| IPH6501 | Antibody-based tetraspecific against NKp46, CD16a, CD122 or IL-2R, and CD20 |

AFM13: anti-CD16A binder is variable domains from human scFv clone LSIV21
Other human scFv anti-CD16A clones: LSIV14, LSIII49, LBIII6, LBIII5, LBIII3, 50NI

| Cite: Reusch et al., mAbs 6:3, | |
|---|---|
| 727-738, May/June 2014 | |
| Binders for V gamma 9, | |
| V delta 2 gene, V delta 1 | |
| gene segments in TCR | |
| LAVA-051 | Lava Therapeutics. bispecific gamma delta T cell engagers (bsTCEs): Vγ9Vδ2 T cell receptor occupancy of |
| | LAVA-051 increased with LAVA-051 dose increases and peripheral blood Vγ9Vδ2 T cells also expressed |
| | higher levels of activation markers after LAVA-051 dosing |
| | LAVA-051 consists of two humanized single domain antibodies (VHH) linked via a short five a.a. G-S linker, |
| | wherein one doamin antibody recognizes the Vδ2 chain of the Vγ9Vδ2 T cell receptor, and the other domain |
| | antibody is specific for CD1d. |
| LAVA-1207 | Binds: the Vδ2 chain of Vγ9Vδ2-T cells and prostate specific membrane antigen (PSMA) |

| | |
|---|---|
| Bruin et al., Clinical Immunology, | set of Vγ9Vδ2-T cell specific VHH (or nanobody) |
| v. 169. 128-138, August 2016 | |

Clones: 5C1, 5D3, 5E3, 5G3, 5F5, 5C7, 5D7, 5E7, 5C8, 5B11, 6A1, 6C1, 6H1, 6E3, 6G3, 6H3, 6C4, 6E4, 6H4, 6F6

| U.S. Pat. Nos. 10,501,540 & | | |
|---|---|---|
| 11,384,145 | | |
| clone 7A5: the variable | | Construct: Vg9 TCR × Her2/neu; that is, (Her2)₂ × Vγ9 bispecific antibody |
| regions of the Vg9gd | | |
| T-cell-specific antibody ( | | |
| GDX012 | GammaDelta | allogeneic, non-engineered, variable delta 1 (V81) gamma-delta (γδ) T cell therapy |
| | Therapeutics | |
| See Pat. Appl. pub.: | | |
| US20220403025; | | |
| US20220290101; | | |
| Anti-CD137 | | |
| (4-1BB/ | | |
| TNFRSF9) | | |
| AGEN2373 | | by Agenus Inc; mAb IgG1; U.S. Pat. Nos. 11,098,117 & 11,242,385 |
| utomilumab | PF-05082566 | Humanized IgG2 mAb; binding site: cysteine-rich domain (CRD)s III and IV |
| Urelumab | BMS-663513 | Human IgG4 mAb; binding site: CRD I; trial by BMS |
| CTX-471 | by Compass | fully human IgG4 mAb |
| | Therapeutics | |
| ADG106 | by Adagene | fully human ligand-blocking, agonistic IgG4 |
| | Suzhou | |
| LVGN6051 | by Lyvgen | IgG1, improved FcγRIIB binding |
| | Biopharma/Merck | |
| | Sharp & Dohme | |
| ATOR-1017 | by Alligator | IgG4 |
| | Bioscience AB | |
| HOT-1030 | by Shanghai | IgG1, abrogated FcγRIIB binding |
| | Huabo | |
| | Biopharmaceutical | |
| | (Huaota)/Eutilex | |
| EU101 | by Eutilex/ | IgG |
| | Zhejiang | |
| | Huahai | |
| | Pharmaceutical | |
| YH004 | by Eucure | IgG1 |
| | (Beijing) | |
| | Biopharma | |
| ADG206 | by Adagene | IgG1, enhanced IgG1 binding, 4-1BB binding sites are masked |
| | Suzhou | |
| PE0116 | by Shanghai | IgG4 |
| | Hyamab Biotech | |
| FS120 | by F-Star | OX40/CD137 mAb² dual agonist bispecific antibody |
| | Therapeutics Inc. | |
| cinrebafusp alfa | PRS-343 | bispecific 4-1BB (Anticalin ®) × HER2 |
| RG7827 | RO 7122290 | FAP × 4-1BBL antibody fusion protein |
| INBRX-105 | ES101 | humanized, bispecific IgG1 targeting PD-L1 × 4-1BB |
| Acasunlimab | GEN 1046, | Fc-silenced IgG1 bispecific PD-L1 × 4-1BB |
| | BNT 311 | |
| MCLA-145 | | bispecific CD137 × PD-L1 |
| Englumafusp | RG6076, | CD19 × CD137 |
| alfa | RO7227166 | |
| Additional bi/multispecific in MAbs. | | |
| 2023; 15(1): 2167189. | | |
| Anti-CD28 | | |
| Theralizumab | TGN1412, | CYTOKINE storm failed human safety trial; humanised monoclonal antibody directed against the human CD28 |
| | CD28- | |
| | SuperMAB, | |
| | >Renamed as | Trial for rheumatoid arthritis |
| | TAB08 | |
| Anti-DNAM-1 | | |
| (CD226 | | |
| or PTA1) | | |
| LY3435151 | | agonistic anti-CD226; NCT04099277 by Eli Lilly; U.S. Pat. No. 11,440,959 |
| Anti-NKp46 | | |
| (NCR1, CD335) | | |
| IPH6501 | | NKp46-Targeting Tetraspecific Antibody-Based Natural Killer Cell Engager Therapeutic Armed with a Non- |
| | | Alpha IL-2 Variant (by Innate Pharma) |
| Trifunctional | | Targeting NKp46, CD16, and a tumor antigen (Cell 177, 1701-1713, Jun. 13, 2019); U.S. Pat No. 10,519,234 |
| NKCE | | (by Innate Pharma) |
| | | Other Innate Pharma owned NKp46 binder patents: U.S. Pat. Nos. 11,001,629, 11,377,492, and 11,267,897 |
| Anti-NKp30 | | |
| | Compass | US20200109195, US20200079867, particularly SEQ ID NOs: 14 and 18 |
| | Therapeutics | |
| Anti-NKG2D | | |
| | NCT05213195 by | US20200360437 by Asko (Suzhou) |
| | Zhejiang Univ. | |
| | | NCT05213195, NCT05247957, NCT04324996, NCT04623944, NCT03415100 |
| Anti-OX40 | | |
| (CD134; | | |
| TNFRSF4) | | |
| INCAGN1949 | | human IgG1 |
| | | Anti-OX40 patents: U.S. Pat. Nos. 11,472,883, 11,447,557, 11,359,028, 11,332,536, 11,136,404, 10,626,181, |
| | | 10,259,882 |
| HFB301001 | | 2nd gen. fullly human IgG1 OX40 agonist antibody by HiFiBio |
| GSK3174998 | | anti-OX40 mAb |
| INBRX 106 | | hexavalent OX40 agonist, active ingredient: hexavalent IgG antibody that targets the human OX40 receptor |
| | | (TNFRSF4) |
| FS120 | by F-Star | OX40/CD137 mAb² dual agonist bispecific antibody |
| | Therapeutics Inc. | |
| Ivuxolimab | PF-04518600 or | humanized agonistic IgG2 monoclonal antibodies against OX40 |
| | PF 8600 | |
| BGB-A445 | | does not block the OX40-OX40L interaction; |
| MOXR0916 | by Genentech | humanized effector-competent agonist IgG1 mAb; completely blocked OX40 binding to OX40L |
| BMS-986178 | | fully human IgG1 agonist mAb |
| Anti-ICOS | | |
| (CD278, | | |
| AILIM, | | |
| CVID1) | | |
| Izuralimab | XmAb23104 by | bispecific PD1 × ICOS |
| | Xencor | |
| MEDI-570 | by Astrazeneca | |
| BMS-986226 | | |
| KY1044 | by Kymab | fully human anti-ICOS IgG1 |
| feladilimab | GSK3359609, | humanized IgG4 anti-ICOS monoclonal |
| | INDUCE-1 | |
| vopratelimab | JTX-2011 | |
| | by Jounce | |
| | Therapeutics | |
| Acazicolcept | | ICOS × CD28 |
| Anti-CD2 | | |
| Siplizumab | TCD601, MEDI- | humanized monoclonal anti-CD2 IgG1 (Humanized form of BTI-322 below) |
| | 507, Hu-BTI-322 | |
| PIT565 | by Novartis | Trispecific CD19 × CD3 × CD2 (NCT05397496) |
| Alefacept | | fusion protein composed of the first extracellular domain of LFA-3 (natural ligand of CD2) fused to the human |
| | | IgG1 hinge, CH2, and CH3 domains |
| BTI-322 | Lo-CD2a | rat anti-human CD2 IgG2b mAb |
| Anti-CD40L | | |
| (CD154) | | |
| ruplizumab | BG 9588, hu5c8 | humanized IgG1k |
| Dazodalibep | HZN-4920 by | antagonistic |
| Dapirolizumab | CDP7657 | anti-CD40L antibody fragment with the Fc moiety substituted by polyethylene glycol |
| pegol | | |
| Tegoprubart | AT-1501 | humanized mAb |
| Frexalimab | SAR441344 | 2nd gen. mAb engineered to lack the domain that activated FcgRIIA |
| Lu AG22515 | APB-A1; by | differentiated anti-CD40L antibody |
| | Lundbeck | |
| Letolizumab | BMS 986004 | humanized mAb (a dimeric fusion protein composed of the C-terminus of the domain antibody (dAb) BMS2h- |
| | | 572-633 targeting CD40L linked to a modified Fc fragment of IgG1) |
| BIIB063 | | high affinity MAb, engineered to reduce Fc effector function and avoid half antibody formation in vivo (IgG4P |
| Anti-CD40 | | |
| (TNFRSF5) | | |
| CDX-1140 | | fully human IgG2 agonistic mAb |
| Sotigalimab | APX005M | humanized IgG1 agonistic mAb |
| 2141-V11 | | human anti-CD40 antibody with modified Fc domain (IgG1), which may induce durable anti-tumor immunity |
| selicrelumab | RO7009789 by | human IgG2 agonistic mAb; CP-870,893 |
| | Roche, or | |
| | CP-870893 | |
| dalnicastobart | LVGN7409 | humanized IgG1 mAb |
| Iscalimab | CFZ533 | fully human IGg1 mAb |
| Dacetuzumab | SGN-40 | humanized IgG1 form of S2C6 (a murine anti-human CD40) |
| BI 655064 | by Boehringer | humanised, nondepleting, antagonistic anti-CD40 |
| | Ingelheim | |
| Bleselumab | ASKP1240 | fully human IgG4 mAb |
| YH003 | | humanized agonistic CD40 IgG2 mAb |
| SL-172154 | SIRPa-Fc-CD40L | Bifunctional CD47 × CD40 |
| Giloralimab | ABBV-927 | agonistic mAb |
| Lucatumumab | CHIR 12.12 or | fully human mAb |
| | HCD122 | |
| Mitazalimab | ADC-1013 | agonistic human IgG1 mAb |
| GEN1042 | | agonistic bispecific CD40 × 4-1BB |
| FFP104 | PG102 | humanised |
| KPL-404 | | humanized IgG4 antibody engineered to bind CD40 without triggering Fc effector functions |
| Cifurtilimab | SEA-CD40 | humanized IgG1 mAb |
| Ravagalimab | ABBV-323 | antagonistic |
| ABBV-428 | | Bispecific CD40 × MSLN |
| BMS-986090 | INX-189 | a dimeric anti-human CD40 VH antagonist domain antibody formatted with a human IgG4 Fc |
| BSI-038 | | agonistic mAb |
| MEDI5083 | | by MedImmune, now Astrazeneca; hexameric CD40L-Fc fusion |
| RG6189 | | a FAP-targeted CD40 agonistic bispecific antibody |
| Inezetamab | AMG 994 | an immunoglobulin G1-scFv_L-lambda2 dimer |

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

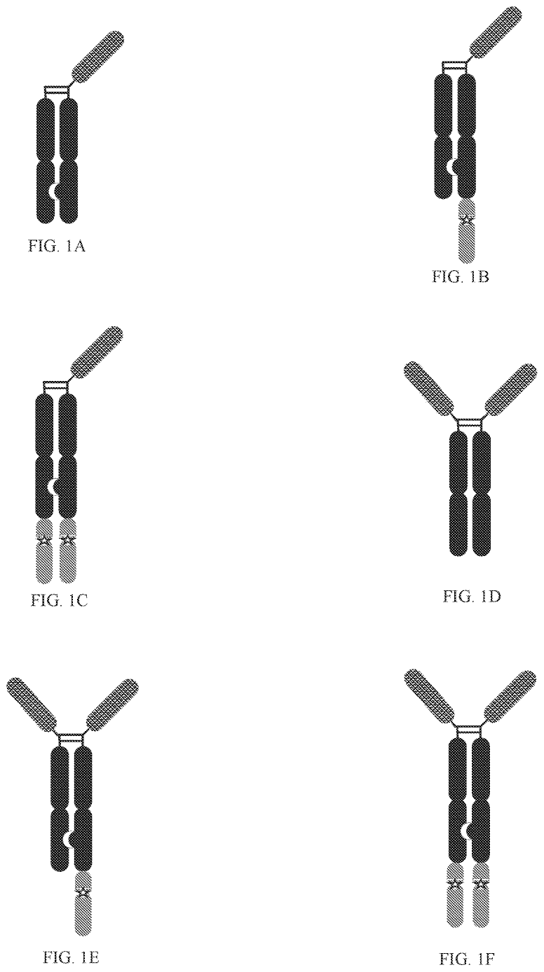

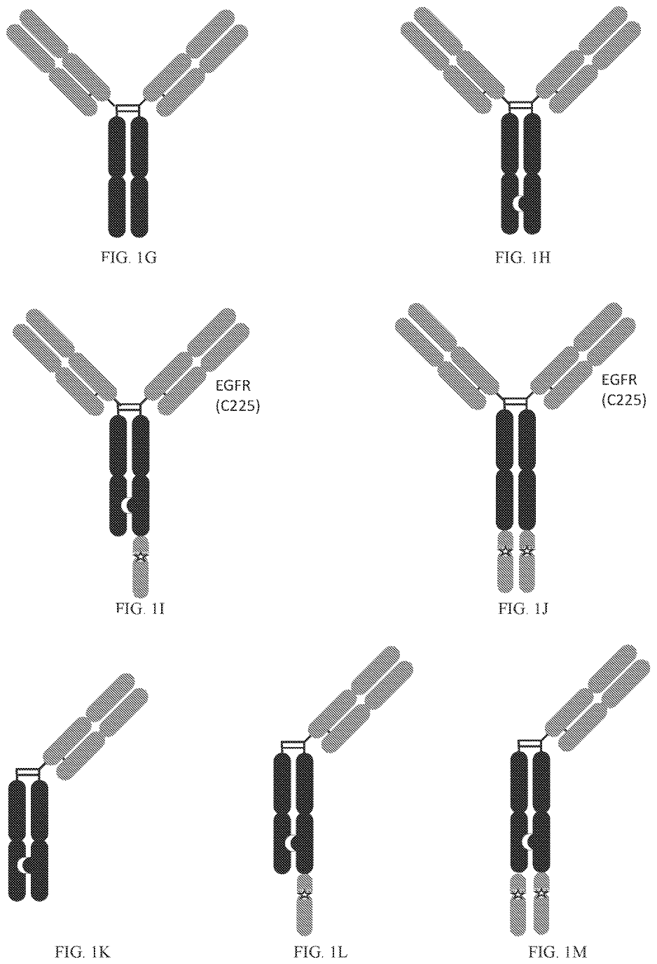

FIG. 1 shows exemplary illustrations of pro-IL-18 fusion proteins (FIGS. 1B-1C, 1E-1F, 1I-1J, 1L-1M) and control fusion proteins (without pro-IL-18; FIGS. 1A, 1D, 1G-H, 1K). All proteins incorporate a human IgG scaffold that is either wild type homodimeric IgG1 as in FIGS. 1D, 1G and 1J or comprising a knob-in-hole (KIH) heterodimeric IgG1 Fc (FIGS. 1A-1C, 1E-1F, 1H-1I, 1J-1M). Unless otherwise noted, all IgG1 Fc domains retain wild type capacity to bind Fc γ Rs and can therefore induce effector mediated Antibody Dependent Cellular Cytotoxicity (ADCC) and Antibody Dependent Cellular Phagocytosis (ADCP). Pro-IL-18, when included in the fusion protein is fused as (a) a singular cassette to either the C-terminal end of the knob (or hole) chain (FIGS. 1B, 1E, 1I and 1L) or (b) as singular cassettes to both the C-terminal ends of the knob and hole chains (FIGS. 1C, 1F, 1J and 1M). Cleavage sites, when present in the pro-IL-18 cassette, are indicated as a star shape. A tumor associated antigen binding domain (TAA), when present, is fused as a single cassette to the N-terminal end of a knob (or hole) chain (FIGS. 1A, 1B, 1C, 1K-1M), or both chains (FIGS. 1D-1J) of the KIH. The TAA binding domain may be a VHH (e.g. EGFR specific clone 9G8; FIGS. 1A-1F) or a fab (e.g. EGFR specific clone C225 from cetuximab; FIGS. 1G-1M).

In some embodiments, the N-terminal end of the CH1 domain of a knobs into holes (KII) heterodimeric IgG1 protein was fused to the C-terminal end of an anti-EGFR VHH or fab. The N-terminal of the KIH protein consisted of (a) a single a VHH on the N-terminal HC knob (FIG. 1A), (b) a single fab on the N-terminal HC knob (FIG. 1K) or (c) a fab on the N-terminal HC knob and HC hole (FIG. 1H). In some embodiments, the N-terminal end of the CHT domain of a homodimeric IgG1 protein was fused to the C-terminal end of a VHH or fab. In some embodiments, the N-terminal of the IgG1 protein consisted of (a) a VHH on both N-terminal HCs (FIG. 1D) and (b) a fab on both N-terminal HCs (FIG. 1G). In some embodiments, the N-terminal of one pro-IL-T8mut2AV was fused to the C-terminal of the CH3 domain of the knob-heavy chain (HC) of a knobs into holes (KIH) heterodimeric IgG1 protein. The N-terminal of the Fc fissions consisted of (a) a VHH clone on the N-terminal HC knob (FIG. 1B), (b) a single fab on the N-terminal HC knob (FIG. 1L) or (c) a VHH on the N-terminal HC knob and on the HC hole (FIG. 1E) or (d) a fab on the N-terminal HC knob and HC hole (FIG. 1I). In all cases, a cleavage site, when present (e.g. the Granzyme B cleavage site, IEQD), was inserted within the IL-18 pro-peptide in place of the endogenous Caspase 1 cleavage site. In some embodiments, the N-terminal of pro-IL-18mut2AV was fused to the C-terminal of the CH3 domain of the HC knob and HC hole of a knobs into holes (KIH) heterodimeric IgG1 protein. The N-terminal of the Fc fusions consisted of (a) a single VHH on the N-terminal HC knob (FIG. 1C) and (b) a single fab on the N-terminal HC knob (FIG. 1M). In all cases, a cleavage site, when present, was inserted within the IL-18 pro-peptide. In some embodiments, the N-terminal of pro-IL-18mut2AV was fused to the C-terminal of the CH3 domain of the homodimeric IgG1 protein. The N-terminal of the Fc fusions consisted of (a) a VHH on both N-terminal HCs or (b) a fab on both N-terminal HCs (FIG. 1M). In all cases, a cleavage site, when present, was inserted within the IL-18 pro-peptide.

### Example 2

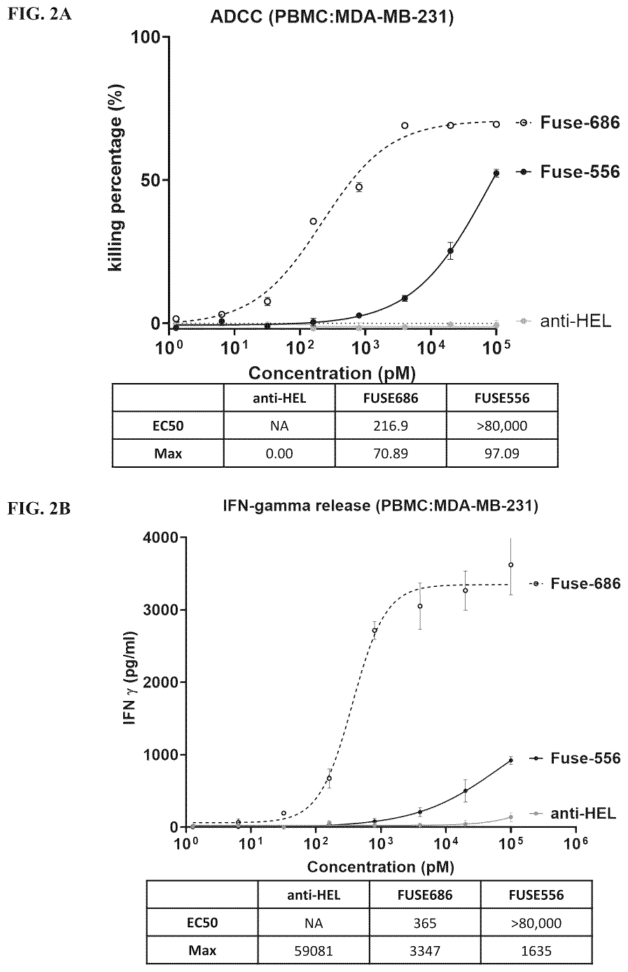

As shown in FIG. 2, we assessed the capacity of FUSE556 (single armed anti-EGFR VHH 9G8-IgG1), and FUSE686 (single armed anti-EGFR VHH 9G8-IgG1-pro-IL-18) to induce (1) PBMC mediated Antibody Dependent Cellular Cytotoxicity (ADCC) of the EGFR+ cell line, MDA-MB-231 (FIG. 2A) and (2) PBMC mediated IFNγ release (FIG. 2B). For FUSE686, pro-IL-18 was fused to the C-terminus FUSE556. Pro-IL-18 incorporates a mutant propeptide upstream of IL-18 to mask/attenuate IL-18 activity. We demonstrated herein that such a propeptide variant masking motif vastly reduced the trans activity of the protein, in some cases by >100,000 fold, compared to wild type IL-18.

To determine the capacity of pro-IL-18 to augment EGFR dependent ADCC/IFNγ release when delivered to both EGFR (tumor cells) and CD16+ cells (NK cells) as a fusion protein, we mixed freshly isolated PBMC with EGFR+ MDA-MB-231 engineered to express firefly luciferase for 48 hours in the presence of either FUSE556 (black closed circles), FUSE686 (black open circles, dashed line), or the negative control Hen Egg Lysozyme specific antibody (anti-HEL; grey closed circles). The PBMC: tumor target ratio was 10:1. However, since NK cells mediate ADCC and are found at a mean frequency of about 10%, the effective NK: tumor target ratio was approximately 1:1. FUSE686 was more potent (EC50-killing) than FUSE556 (>350 fold) and induced a higher maximum killing (~40% higher). Summary tables of potency and maximum activity for the test articles are shown below each graph. With relation to IFNγ release, FUSE686 was more potent (EC50-IFNγ release) than FUSE556 (>300 fold) and induced a higher maximum IFNγ release (~3.5 fold higher). Given that pro-IL-18 was previously found to have little to no activity when non-targeted (trans-activity), we interpret the results as indicating that pro-IL-18, when targeted to CD16 via wild type IgG1, can bind to the IL-18R complex when bound in close proximity to it (i.e. CD16 on the same cell). Such targeted/proximity related activity has been previously terms as occurring in cis.

### Example 3

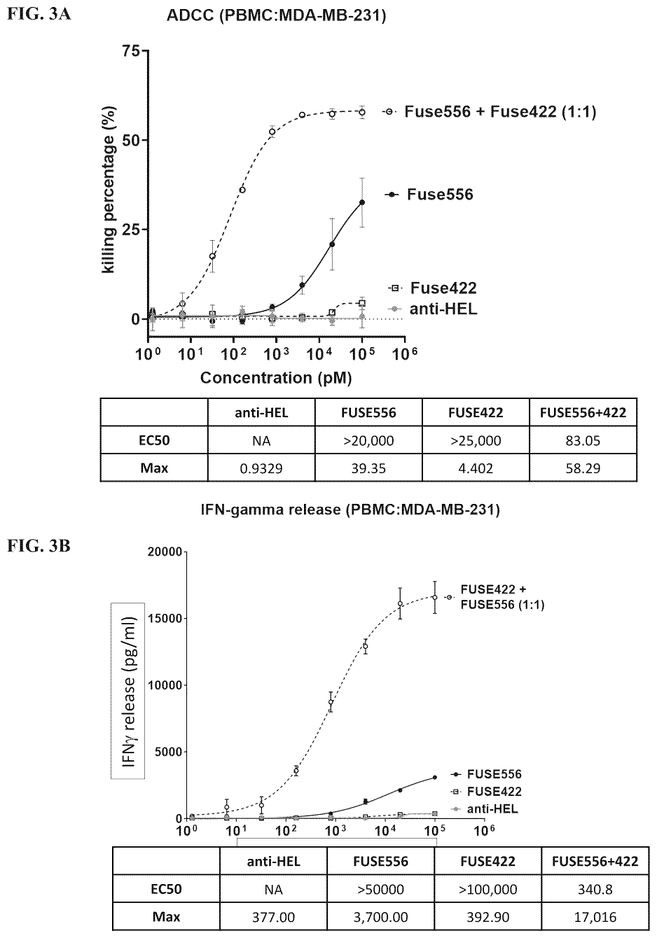

As shown in FIG. 3, we assessed the capacity of FUSE556 (single armed anti-EGFR VHH 9G8-IgG1; targets both EGFR and FcγR), and the combination (1:1, molar equivalence) of FUSE566 and FUSE422 (IgG1-pro-IL-18; only targets FcγR) to induce (1) PBMC mediated Antibody Dependent Cellular Cytotoxicity (ADCC) of the EGFR+ cell line, MDA-MB-231 (FIG. 3A) and (2) PBMC mediated IFNγ release (FIG. 3B). For FUSE422, pro-IL-18 was fused to the C-terminus of the Fc domain of IgG1. FUSE422 is shown herein to be >100,000 fold attenuated in trans compared to wild type IL-18.

To assess the capacity of pro-IL-18 to augment EGFR dependent ADCC/IFNγ release induced by FUSE566 when co-delivered to CD16+ cells (NK cells) as Fc-pro-IL-18 (FUSE422), we mixed freshly isolated PBMC with EGFR+ MDA-MB-231 engineered to express firefly luciferase for 48 hours in the presence of either FUSE556 (black closed circles), the 1:1 molar combination of FUSE556 and FUSE422 (black open circles, dashed line), FUSE422 alone (black open boxes; dashed line) or the negative control Hen Egg Lysozyme specific antibody (anti-HEL: grey closed circles). The PBMC: tumor target ratio was 30:1. However, since NK cells mediate ADCC and are found at a mean frequency of about 10%, the effective NK: tumor target ratio was approximately 3:1.

The combination of FUSE556 and FUSE422 was more potent (EC50-killing) than FUSE556 (>250 fold) and induced a higher maximum killing (~100% higher). Summary tables of potency and maximum activity for the test articles are shown below each graph. Neither FUSE422 nor anti-HEL induced appreciable PBMC (NK) mediated killing of MDA-MB-231. FUSE422 induced a small degree of killing (<5% maximum killing relative to the combination of FUSE556 and FUSE422). This was likely due to targeting of pro-IL-18 to “allo-reactive” NK cells such that the small degree of killing was dependent on “lack of self” and/or expression of ligands for activation receptors such as NKG2D. Indeed, MDA-MB-231 has been reported to express MIC-B, a ligand for NKG2D (see e.g., onlinelibrary.wiley.com/doi/full/10.1002/ijc.28174; www.nature.com/articles/bjc201779; link.springer.com/article/10.1007/s12192-014-0532-5)

Results related to IFNγ release were similar to cytotoxicity except that FUSE422 induced no cytokine release over background or the negative control anti-HEL antibody. The combination of FUSE556 and FUSE422 was more potent (EC50-killing) than FUSE556 (>150 fold) and induced a higher maximum killing (~700% higher). Summary tables of potency and maximum activity for the test articles are shown below each graph.

Taken together with the results from FIG. 1, our data indicates that pro-IL-18, when targeted to CD16 via wild type IgG1, either as an Fc fusion alone (no intrinsic ADCC capacity) or an EGFR targeted Fc fusion (binds both CD16 and EGFR to induce ADCC), can bind to and activate the IL-18R complex in cis.

### Example 4

As shown in FIG. 4, we assessed the impact of targeting pro-IL-18 to PD1 on the activation of purified T cells from a healthy human donor when mixed with DC from a second healthy human donor at a T:DC ratio of 10:1. IFNγ release at 72 hours of co-culture was used as the readout of activation. DC were differentiated in-vitro from PBMC using GM-CSF and IL-4 for 7 days. The following proteins were tested: Human recombinant IL-18 (positive control; black closed stars), FUSE691 (nivolumab; black closed circles), FUSE645 (EGFR targeted IgG1-pro-IL-18; non-targeted in this system; gray closed circles-dashed line) and FUSE694 (nivolumab-pro-IL-18: black open circles-dashed line). Consistent with our finding using the HEK-Blue-IL-18 reporter cell line as described herein, trans activating pro-IL-18 used in the non targeted FUSE645 was vastly attenuated relative to human recombinant IL-18 both as a degree of potency (~1000-fold difference in EC50-IFNγ release) and maximum IFNγ release (~10 fold). Targeting pro-IL-18 to PD1 via fusion of pro-IL-18 to the C-terminus of nivolumab (FUSE694) resulted in about a 2-fold and 20-fold increase in potency relative to FUSE645 and FUSE691 (nivolumab), respectively. Importantly, maximum T cell mediated IFNγ release induced by FUSE694 increased by 7-fold and 3.5-fold relative to FUSE645 and FUSE691 (nivolumab), respectively. Summary tables of potency and maximum activity for the test articles are shown below each graph. This data indicates that although pro-IL-18 is largely non-functional when non-targeted (i.e., acting in trans), when delivered to PD1 and hence placed in close proximity to the IL-18R complex expressed by PD1+ T cells augments IFNγ release, our measure of T cell activation in this system. As described previously for targeting of CD16, the data is supportive of pro-IL-18 acting is cis when targeted to T cells co-expressing PD1 and the IL-18R complex. Given that PD1+ T cells within TIL have been reported to constitute a major fraction of the tumor specific T cells within tumors. PD1 dependent cis activity related to pro-IL-18 may promote the cytotoxic potential, proliferation, survival and memory formation of tumor specific T cells without the indiscriminate activation of other cells that may contribute to systemic toxicity.

### Example 5

As shown in FIG. 5, we assessed the capacity of FUSE556 (single aimed anti-EGFR VHH 9G8-IgG1), and FUSE516 (single armed anti-EGFR VHH 9G8-IgG1-pro-IL-18) to induce PBMC mediated Antibody Dependent Cellular Cytotoxicity (ADCC) of the EGFR+ cell line, MDA-MB-231. For FUSE516, pro-IL-18 included the Granzyme B cleavage site, IEQD, within the pro-peptide such that in addition to the CD16 targeted cis related activity of pro-IL-18, exposure to Granzyme B (released by NK cells mediating ADCC) would release the active mature form (18 kDa) of IL-18. Freshly isolated PBMC were mixed together with MDA-MB-231 engineered to express firefly luciferase for 48 hours in the presence of either FUSE556 (black closed circles), FUSE516 (black open circles, dashed line), FUSE442 (non targeted IgG1-pro-IL-18 control; grey closed circles) or the negative control Hen Egg Lysozyme specific antibody (anti-HEL: grey closed circles). The assay was repeated with PBMC from four different normal human donors as indicated in FIGS. 5A-5D. The PBMC: tumor target ratio was 30:1. However, since NK cells mediate ADCC and are found at a mean frequency of about 10%, the effective NK: tumor target ratio was approximately 3:1. For all donors, FUSE516 was more potent (EC50-killing) than FUSE556 (ranging from ~100 fold to >1000 fold) and induced a higher maximum killing (ranging from about 30% to 800% higher). Summary tables of potency and maximum activity for the test articles are shown below each graph.

### Example 6

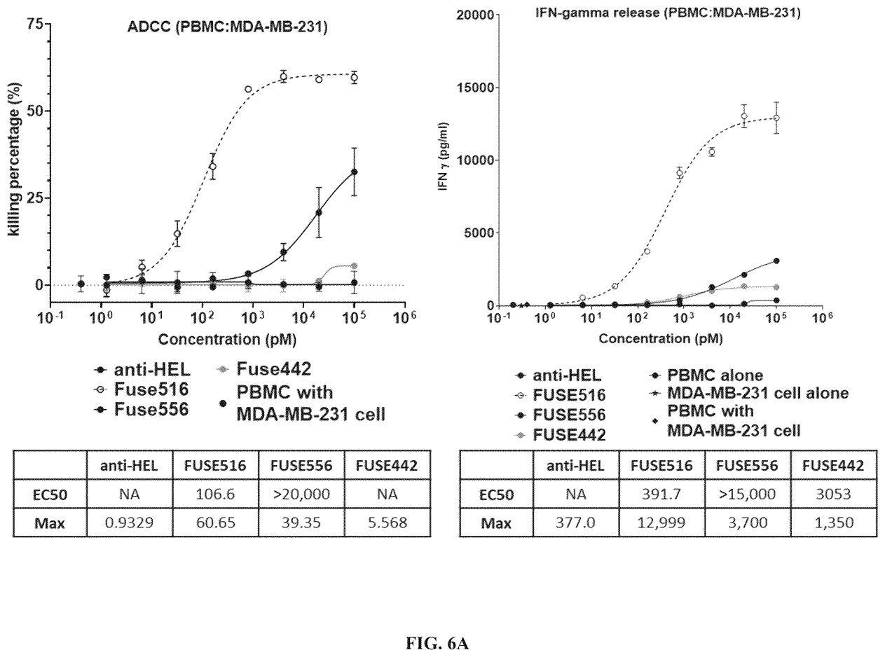

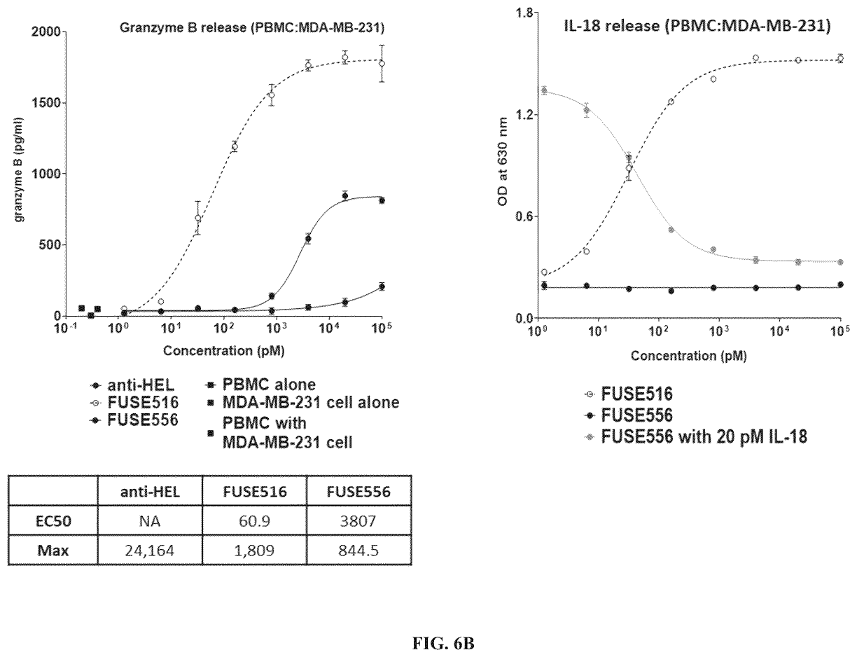

As shown in FIG. 6, we assessed the capacity of FUSE556 (single armed anti-EGFR VHH 9G8-IgG1; black closed circles), and FUSE516 (single armed anti-EGFR VHH 9G8-IgG1-pro-IL-18; black open circles, dashed line) to induce several readouts mediated by freshly isolated PBMC in co-culture with the EGFR+ cell line, MDA-MB-231 after 48 hours of co-culture at an E:T of 30:1. This corresponds to an effective NK: tumor target ratio of approximately 3:1. FUSE442 (non targeted IgG1-pro-IL-18 control; grey closed circles) and the anti-HEL (grey closed circles) were used as negative controls. The functional readouts were as follows: (a) ADCC (FIG. 6A), IFNγ release (FIG. 6B), Granzyme B release (FIG. 6C), and IL-18 release/consumption (FIG. 6D). We observed potent PBMC mediated killing and IFNγ release induced by FUSE516. For ADCC, IFNγ release and Granzyme B release, the potency (EC50-killing, EC50-IFNγ release and EC50-Granzyme B release, respectively) associated with FUSE516 was >200-fold, >50-fold, ~50-fold greater than FUSE556. Maximum induction for each readout was about 100% (ADCC), 500% (IFNγ) and 250% (Granzyme B) for FUSE516 versus FUSE556. Summary tables of potency and maximum activity for the test articles are shown below each graph. The non-targeted pro-IL-18 control, FUSE442, induced very minimal killing of target cells (EC50-killing>25 nM and max killing<6%), which was independent of ADCC and likely related to the recognition of ligands for NK activation receptor(s) known to be expressed on MDA-MB-231 such as MICA/B (Shen et al., Cell Death and Disease. 2017) or minor degree of T cell mediated alloreactivity.

We also measured the magnitude of IL-18 released into the supernatant of the assay described above. Here, the IL-18 reporter cell line, HEK-Blue-IL-18, was exposed to assay supernatants and SEAP activity measured as a surrogate for IL-18 activity. FIG. 6D illustrates the results such that FUSE516 (black open circles, dashed line) that incorporates a Granzyme B cleavage site between its Fc domain and IL-18 but not FUSE556 (black closed circles) released IL-18 is a dose dependent manner in co-culture with PBMC and MDA-MB-231. To estimate the degree of IL-18 consumed by NK cells during the ADCC process, we added rhIL-18 to the PBMC/tumor cell/FUSE556 co-culture (grey closed circles). Here we observed a FUSE556 dose dependent decrease in IL-18 in the assay supernatant. Given that FUSE516 induced strong tumor cell killing and IFNγ release, we interpret that data as indicating that NK cells exposed to FUSE516 consume more IL-18 than those exposed to FUSE556. As such, the magnitude of IL-18 estimated to be released by FUSE516 in FIG. 5D is likely underestimated.

### Example 7

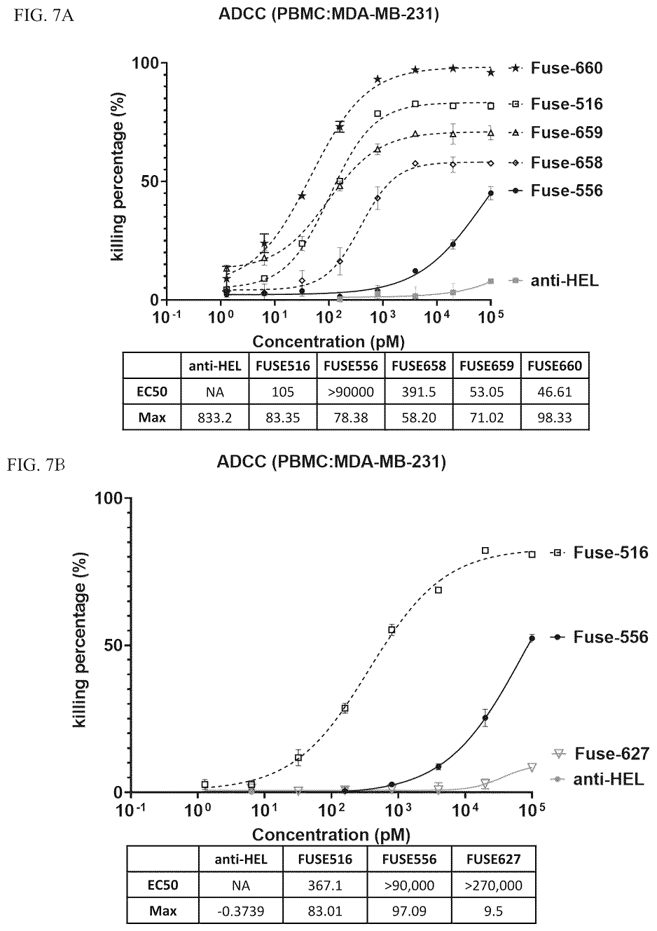

As shown in FIG. 7, we assessed the capacity of FUSE556 (single armed anti-EGFR VHH 9G8-IgG1), and variants of pro-IL-18 fused to the C-terminus of FUSE566 with different protease cleavage sites between the Fc domain and pro-IL-18 to induce PBMC mediated Antibody Dependent Cellular Cytotoxicity (ADCC) of the EGFR+ cell line, MDA-MB-231. The different protease sites tested in FIG. 7A were Granzyme A (FUSE658), Granzyme B (FUSE516). MMP2/9 (FUSE659), and the combination of Granzyme A, Granzyme B and MMP2/9 (FUSE660). Granzyme A and B would be expected to be released by NK cells mediating ADCC whereas MMP2 has been reported as expressed/secreted by MDA-MB-231 (www.ncbi.nhm.nih.gov/pmc/articles/PMC5661385). Also tested was FUSE627 that contains the PGLALA mutation in the Fc domain to abolish targeting to FcγRs (FIG. 7B) and hence abrogate bridging between FcγR+NK cells and EGFR+ tumor cells, which is the driver of ADCC in the reported system.

To assess the impact of the aforementioned protease cleavage sites on the capacity of pro-IL-18 to augment EGFR dependent ADCC, we mixed freshly isolated PBMC with EGFR+ MDA-MB-231 cells engineered to express firefly luciferase for 48 hours in the presence of either FUSE556 (black closed circles; FIGS. 7A-7B), FUSE658 (black open diamonds, dashed line; FIG. 7A), FUSE516 (black open boxes; dashed line; FIGS. 7A-7B), FUSE659 (black open triangles: dashed lines; FIG. 7A), FUSE660 (black closed stars, dashed lines; FIG. 7A), or the negative control Hen Egg Lysozyme specific antibody (anti-HEL; grey squares). The PBMC: tumor target ratio was 30:1 (FIG. 7A) or 10:1 (FIG. 7B). Since NK cells mediate ADCC and are found at a mean frequency of about 10%, the effective NK: tumor target ratio was approximately 3:1 (FIG. 7A) and 1:1 (FIG. 7B).

As seen in FIG. 7A, we found that the rank order of killing potency as it relates to cleavage site(s) was (1) the combination of Granzyme A, Granzyme B and MMP2/9 (FUSE660), which was about 2-fold greater than both (2) Granzyme B (FUSE516) and (3) MMP2/9 (FUSE659), while it was about 3-fold greater than (4) Granzyme A (FUSE658). The rank order as it relates to maximum killing was the same except that Granzyme B (FUSE516) was greater than MMP2/9 (FUSE659). In each case, the difference in maximum killing between each test article was about 10%. That the combination of cleavage sites for all three of Granzyme A, Granzyme B and MMP2/9 was superior to any single cleavage site alone is consistent with the inducible trans activity of pro-IL-18. To semi-quantitate the degree of IL-18 dependent tumor cell killing mediated in trans in the absence of effector to tumor bridging, we compared FUSE516 to FUSE627 (FIG. 7B). FUSE627 is identical to FUSE516 except it contains the PGLALA mutation reported to abrogate binding of IgG1 to FcγRs pubmed.ncbi.nlm.nih.gov/27578889. As such, the pro-IL-18 domain in FUSE627 is not targeted to FcγRs expressed on NK cells and does not act in cis. Further, FUSE627 cannot bridge FcγRs expressed on NK cells to EGFR expressed on MDA-MB-231. Any activity associated with FUSE627 (gray open inverted triangles) would be interpreted as stemming from the trans activity of either pro-IL-18 or Granzyme B released mature IL-18. Such release of Granzyme B would likely result from the interaction of “allo-reactive” NK cells with MDA-MB-231 tumor cells that (a) do not express HLA molecules to which the NK cells were educated and/or (b) express MIC-B, a ligand for activation receptor, NKG2D; www.nature.com/articles/bjc201779; link.springer.com/article/10.1007/s12192-014-0532-5). Alloreactivity dependent release of Granzyme B would then be expected to release mature IL-18 and in turn augments NK mediated killing of the allogeneic target. MDA-MB-231

Unless otherwise specified, in the context of an IL-18 fusion protein disclosed in this invention, any interaction between IL-18 and its ligand, the IL-18 receptor complex, that is not dependent on a targeting moiety to facilitate the interaction between IL-18 and its receptor at close proximity, is considered a trans interaction; and in contrast, any interaction between IL-18 within an IL-18 fusion protein and its receptor that requires the targeting moiety also within the fusion protein to facilitate an interaction between IL-18 and its receptor is considered as occurring in cis. In such a cis based interaction, the targeted molecule (e.g., PD-1) is expressed on the same cell as the IL-18R complex (e.g. an NK cell). Generally speaking, in cellular biology, the term “cis” refers to the interaction between two molecules such as proteins expressed on the same cell. For example the interaction between B7-1 and PD-L1 on T cells is a cis interaction. For multispecific proteins, those that bind two proteins on the same cell simultaneously are also referred to as having a cis interaction. In cellular biology, the term “trans” refers to the interaction between two molecules such as proteins on different cells. For example, the interaction between the prototypical interaction of the T cell Receptor (TCR) and its ligand, an MHC-I/peptide complex, is a trans interaction. For multispecific proteins, those that bind one protein on one cell and a second protein on a separate cell. i.e., forming a “bridge” between the two cells, would be considered as occurring in trans. Further, interaction between a soluble protein and a cell surface protein, although not forming a cellular bridge, is accepted to fall within the definition of a trans interaction. In the therapeutics landscape, soluble proteins that naturally interact with their ligand in trans are often engineered to be of such low affinity that they no longer bind their ligand unless brought into close proximity of it via a targeting moiety that binds another protein on the same cell. This scenario allows for cell targeted activity of the soluble protein and is categorized as a cis interaction.

As previously observed, FUSE516 was more potent (EC50-killing) than FUSE556 (~10 fold) and induced a higher maximum killing (~15% higher). In contrast, FUSE627 was observed to induce very weak killing consistent with augmentation of “background” alloreactive cytotoxicity that results in the of small amounts of Granzyme B into the media, which can then release fully active IL-18 from FUSE627. Summary tables of potency and maximum activity for the test articles are shown below each graph.

### Example 8

As shown in FIG. 8, we assessed the capacity of FUSE556 (single armed anti-EGFR VHH 9G8-IgG1; black closed circles), and FUSE516 (single armed anti-EGFR VHH 9G8-IgG1-pro-IL-18; black open circles, dashed line) to induce two readouts (ADCC and IFNγ release) mediated by expanded NK cells in co-culture with the EGFR+ cell line, MDA-MB-231 after 48 hours of co-culture at E:T ratios of 1:1 and 1:5. An E:T ratio of 5:1 was also tested, and yielded similar results to 1:1 and is not shown. The anti-HEL antibody (grey closed circles) was used as the negative control and no functional activity was observed with this protein. The functional readouts were as follows: (a) ADCC (FIGS. 8A and 8B) and IFNγ release (FIGS. 8C and 8D). Effector populations of NK cells were expanded from PBMC isolated from two distinct healthy donors. The data shown is calculated based on the average of the two donors.

At both E:T ratios, FUSE516 was more potent than FUSE556. For ADCC, (a) the difference in potency between FUSE516 and FUSE556 was approximately 60-fold and (b) the difference in maximum killing between FUSE516 and FUSE556 ranged between 30-50%.

For IFNγ release, the difference in (a) potency between FUSE516 and FUSE556 ranged between 40-fold and >130-fold and may be modesty inversely related to E:T and (b) maximum IFNγ release between FUSE516 and FUSE556 ranged between 250% and 380%. Summary tables of potency and maximum activity for the test articles are shown below each graph. FUSE516 is depicted as open circles and FUSE556 as closed squares. The EC50 values for killing and IFNγ release obtained using an E:T ratio of 5:1 are included in this analysis. This data generated using expanded NK cells of >95% purity (data not shown) is highly similar to that observed with PBMC as effector cells (see FIG. 6), thus providing support that the effector cells within PBMC mediating killing of tumor targets and releasing IFNγ are NK cells. The difference in the magnitude of IFNγ release between FUSE516 and FUSE566 is impressive and suggests that FUSE516 may promote enhanced anti-tumor activity not only via ADCC but also via the promotion of innate and adaptive immunity including tumor antigen presentation and bystander killing.

### Example 9

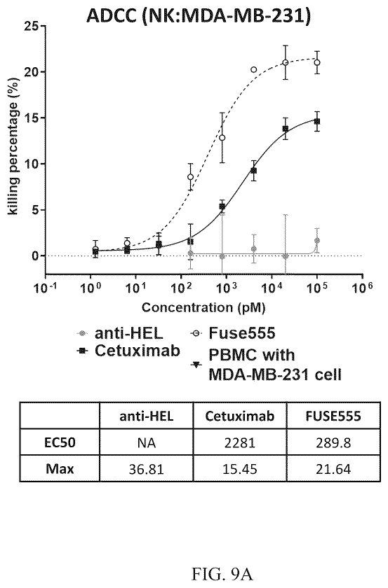

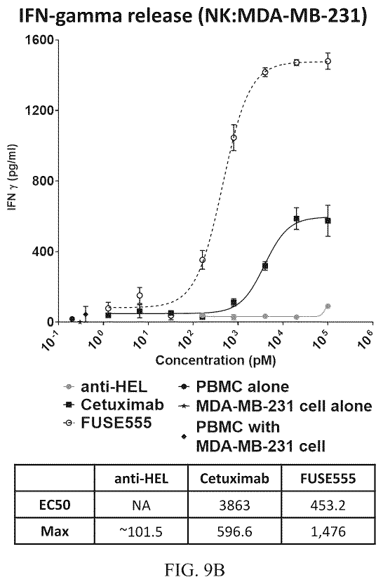

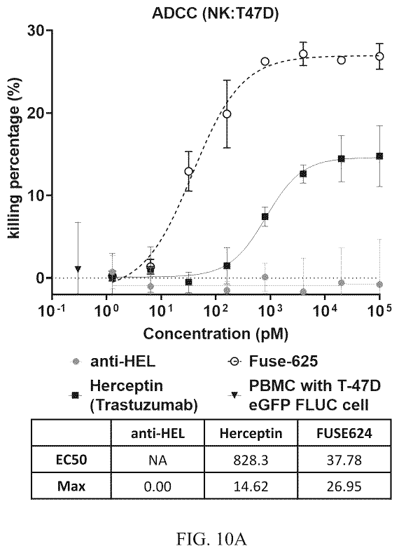

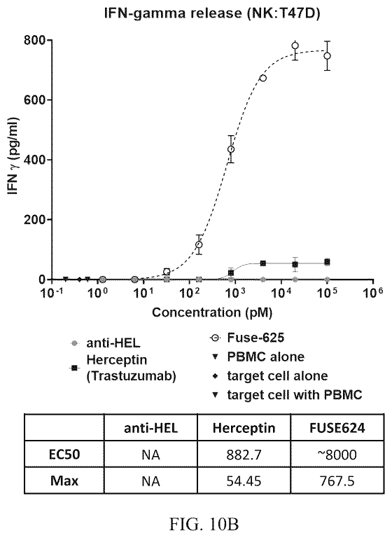

As shown in FIG. 9, we assessed the capacity of FUSE555 (dual armed anti-EGFR Fab C225-IgG1-pro-IL-18; black open circles, dashed line), and cetuximab (black closed squares) to induce two readouts (ADCC and IFNγ release) mediated by freshly isolated PBMC in co-culture with the EGFR+ cell line, MDA-MB-231 after 40 hours of co-culture at an E:T ratio of 2:1. The anti-HEL antibody (grey closed circles) was used as the negative control and no functional activity was observed with this protein. The functional readouts were as follows: (a) ADCC (FIG. 9A) and IFNγ release (FIG. 9B). Effector populations of NK cells were expanded from PBMC isolated from two distinct healthy donors. The data shown is calculated based on the average of the two donors.

For ADCC, (a) the difference in potency between FUSE555 was approximately 3-fold more potent than Cetuximab and (b) maximum killing induced by FUSE555 was about 40% greater than Cetuximab.

For IFNγ release (see FIG. 9B), the difference in (a) potency between FUSE555 and Cetuximab was about 3-fold and (b) maximum IFNγ release associated with FUSE555 was about 200% higher relative to cetuximab. FUSE555 is depicted as open circles and Cetuximab as closed squares. Summary tables of potency and maximum activity for the test articles are shown below each graph. The difference in the magnitude of IFNγ release between FUSE555 and Cetuximab is highly appreciable. Since IFNγ can impact anti-tumor activity by promoting innate and adaptive immune responses, we potentially consider FUSE555 a far superior therapeutic compared to Cetuximab. FUSE555 may promote enhanced anti-tumor activity not only via ADCC but also via the promotion of innate and adaptive immunity including tumor antigen presentation and bystander killing.

### Example 10

As shown in FIG. 10, we assessed the capacity of FUSE624 (dual armed anti-HER2 Fab IgG1-pro-IL-18 (black open circles, dashed line), and trastuzumab (black closed squares) to induce two readouts (ADCC and IFNγ release) mediated by freshly isolated PBMC in co-culture with the HER2+ cell line, T47D after 24 hours of co-culture at an E:T ratio of 5:1. The anti-HEL antibody (grey closed circles) was used as the negative control and no functional activity was observed with this protein. The functional readouts were as follows: (a) ADCC (FIG. 10A) and IFNγ release (FIG. 10B).

For ADCC, FUSE624 was (a) approximately 30-fold more potent than trastuzumab and (b) maximum killing was about 2-fold greater versus trastuzumab.

For IFNγ release, (a) FUSE624 was about 10-fold more potent than trastuzumab and (b) maximum IFNγ release induced by FUSE624 was approximately 16-fold higher than trastuzumab. FUSE624 is depicted as open circles and trastuzumab as closed squares. Summary tables of potency and maximum activity for the test articles are shown below each graph. The difference in the magnitude of IFNγ release between FUSE624 and trastuzumab is very impressive. Since IFNγ can impact anti-tumor activity by promoting innate and adaptive immune responses, we potentially consider FUSE624 a far superior therapeutic compared to trastuzumab. FUSE624 may promote enhanced anti-tumor activity not only via ADCC but also via the promotion of innate and adaptive immunity including tumor antigen presentation and bystander killing.

### Example 11

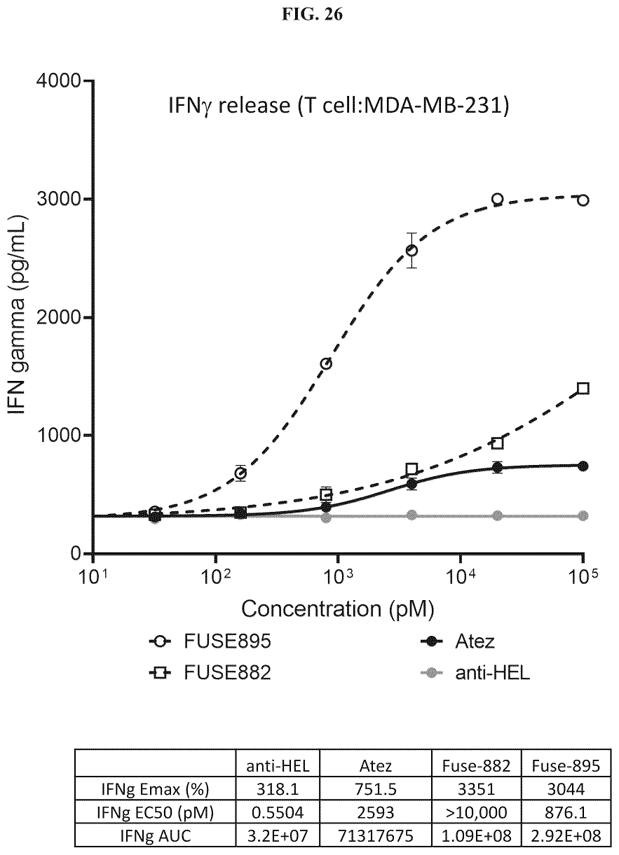

As shown in FIG. 26, we assessed the capacity of an anti-PDL1 antibody incorporating a pro-IL-18amut2 variant, designed so that it cannot be cleaved by Granzyme B, to induce T cell mediated IFNγ release. In this context, any enhancement of IFNγ release relative to the impact of anti-PDL1 alone (atezolizumab) is interpreted as the result of (1) a cis based interaction between pro-IL-18amut2 and the IL-18R complex expressed on PD-L1+ activated T cells and/or (2) pro-IL-18amut2 decorated on PD-L1+ activated T cells interacting with IL-18R complexes on neighboring IL-18R+ T cells. The assay system used consisted of human T cells preactivated for 72 hours with CD3/CD28 coated beads to upregulate PDL1 (and PD1) expression mixed with MDA-MB-231 (ROR1+) stably expressing firefly luciferase and eGFP at an E:T ratio of 10:1 and 10 pM of a ROR1 x CD3 bsAb (Fuse608). Fuse608 served to create an artificial synapse between the T cells and ROR1+ tumor cells and deliver a suboptimal signal via the TCR/CD3 complex that resulted in the background IFNγ release. 10 pM was calculated as the concentration of Fuse608 to induce 20% maximal IFNγ release. A titration of the following test articles was added to the aforementioned T cell/tumor cell co-culture system and IFNγ release measured after 48 hours: (1) atezolizumab (black closed circles), (2) Fuse882 (open square, dashed line) consisting of a single non-cleavable pro-IL-18amut2 fused to the C-terminal knob of a knobs into holes human IgG1 version of atezolizumab, (3) Fuse895 consisting of a single non-cleavable pro-IL-18amut2 fused to the N-terminal knob of a knobs into holes human IgG1 version of monovalent atezolizumab whereby the anti-PDL1 fab was fused to the N-terminal hole (open circles, dashed line) or (4) an anti-HEL antibody (grey circles) which served as the isotype matched negative control. As shown in the non-linear x-y plot depicting concentration of test article versus the magnitude of IFNγ release, a highly appreciable enhancement of IFNγ release was observed with Fuse895 relative to atezolizumab at the orders of about 4-fold and 3-fold for maximum IFNγ release and the potency (EC50) of IFNγ release, respectively. A summary table of EC50, Emax and AUC (area under the curve) is shown beneath of x-y plot.

### Example 12

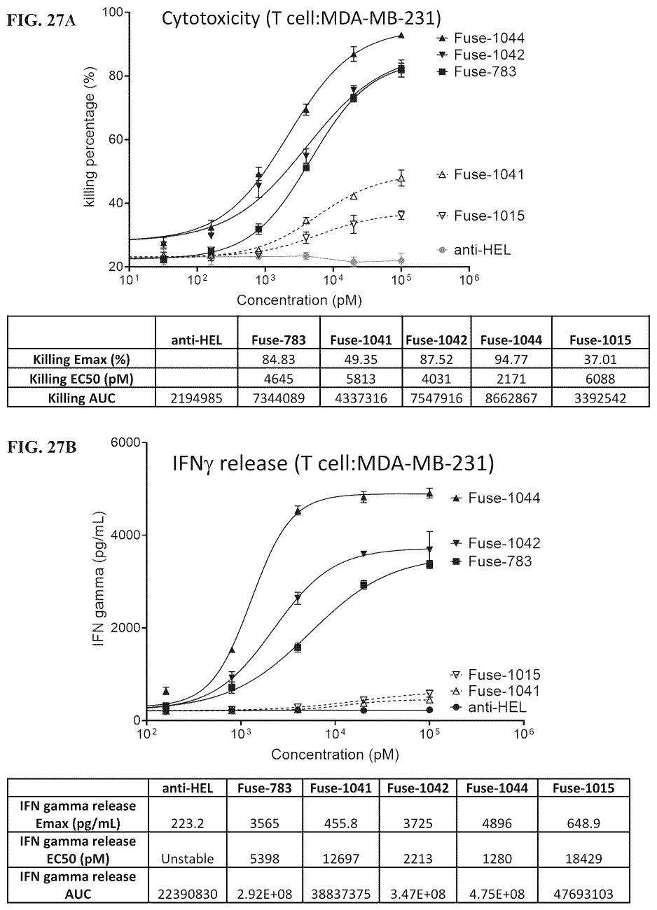

As shown in FIG. 27A-27B, we assessed the capacity of an anti-PD1 antibody, incorporating one of two pro-IL-18 variants (pro-IL18amut2 or pro-IL18amut9), designed so that it cannot be cleaved by Granzyme B, to induce T cell mediated tumor cell killing (FIG. 27A) and IFNγ release (FIG. 27B). In this context, any enhancement of tumor cell killing and IFNγ release relative to the impact of anti-PD1 alone is interpreted as the result of a cis based interaction between pro-IL-18 and the IL-18R complex expressed on PD-1+ activated T cells. The assay system used consisted of purified human T cells mixed with MDA-MB-231 (ROR1+) stably expressing firefly luciferase and eGFP at an E:T ratio of 10:1 and 10 pM of a ROR1 x CD3 bsAb (Fuse608). As described above. Fuse608 served to create an artificial synapse between the T cells and ROR1+ tumor cells and deliver a suboptimal signal via the TCR/CD3 complex.

A titration of test articles was added to the aforementioned T cell/tumor cell co-culture system. Tumor cell killing and IFNγ release were measured after 72 hours. The test articles assessed were: (1) anti-PD-1 with pro-IL-18amut2 incorporated on the C-terminal knob (Fuse783; closed square), (2) anti-PD-1 with pro-IL-18amut2 incorporated on the N-terminal hole (Fuse1042; closed reverse triangle), (3) anti-PD-1 with pro-IL-18amut9 incorporated on the N-terminal hole (Fuse1044; closed triangle), (4) anti-PD1 (Fuse1041; open triangle, dashed line) and (5) Fuse1015 (open reverse triangle, dashed line), which was equivalent to Fuse1042 except specific for mouse PD-1 and hence served as the none-targeted pro-IL-18amut2 control. An anti-HEL antibody (grey closed circle) was used as the isotype matched negative control. FIG. 27A is the non-linear x-v plot depicting concentration of test article versus the magnitude of tumor cell killing. Relative to anti-PD-1 alone, the three test articles incorporating pro-IL-18 were observed to induce between 60-80% more killing (based on Emax), with pro-IL-18amut9 being the most potent. FIG. 27B is a non-linear x-y plot depicting concentration of test article versus the magnitude of IFNγ release. In this setting, the three test articles incorporating pro-IL-18 induced stronger IFNγ release relative to anti-PD-1 alone that ranged in enhanced activity between 8-11 fold (based on Emax) and at least 3-10 fold (based on EC50). As observed for tumor cell killing, pro-IL-18amut9 was the most potent.

### Example 13

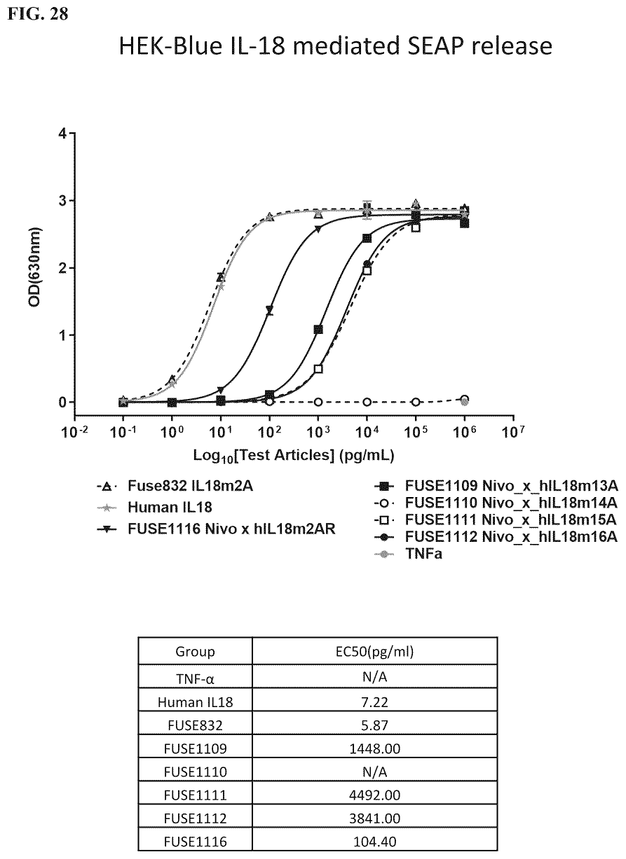

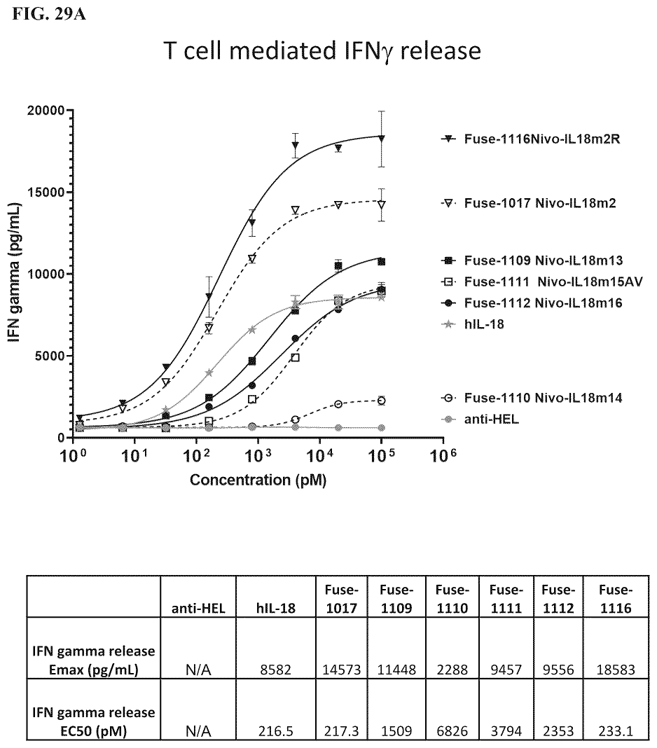

As shown in FIG. 28, we assessed the capacity of several IL-18 variants to induce non-targeted trans based release of SEAP from HEK-Blue-IL-18. As previously indicated, HEK-Blue-IL-18 expresses the human IL-18 receptor complex and responds to exposure to IL-18 via the release of SEAP due to the integration of a transgene encoding SEAP downstream of an AP1/NFκB minimal promoter. HEK-Blue-IL-18 were culture with a titration of test articles for 24 hours. The test articles examined were the following IL-18 variants fused to an IgG1-LALA backbone targeted to PD-1 using the paratope from nivolumab: IL18mut2 (Fuse832; open triangle, dashed line), IL18mut2R (Fuse1116: reverse closed triangle), IL18mut13 (Fuse1109; closed square). IL18mut14 (Fuse1110; open circle, dashed line), IL18mut15 (Fuse1111; open square, dashed line) and IL18mut16 (Fuse1112; closed circle). TNFα (closed gray circle) was used as the negative control and recombinant human IL-18 was used as the reference compound (gray star). Based on the non-linear x-y plot of shown, the rank order of IL-18 induced SEAP release was Fuse832>Fuse1116>Fuse1109>Fuse1111/Fuse1112>Fuse1110. The human recombinant IL-18 reference exhibited similar activity to Fuse832. A summary table of EC50s for the test articles are shown below the graph.

### Example 14

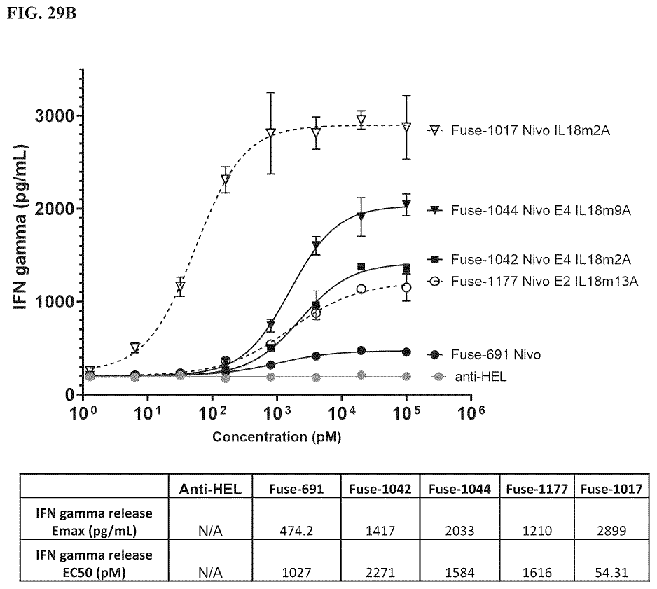

As shown in FIGS. 29A and 29B, we assessed the capacity of several IL-18 variants targeted to human PD-1 to induce cis based release of IFNγ from human PBMC derived from a healthy human donor (FIG. 29A) or human T cells (FIG. 29B). For FIG. 29A, PBMC were treated activated with the CD3 specific antibody, pelicluster CD3, for 48 hours to induce upregulation of PD-1 expression. After washing, these PBMC were culture in a titration of test articles in the presence of a suboptimal concentration of pelicluster CD3 and IFNγ released into the supernatant assessed after 72 hours. The test articles examined were the following IL-18 variants fused to an IgG1-LALA backbone targeted to PD-1 using the paratope from nivolumab: IL18mut2 (Fuse1017; reverse open triangle, dashed line), IL18mut2R (Fuse 1116: closed reverse triangle), IL18mut13 (Fuse1109; closed square). IL18mut14 (Fuse1110; open circle, dashed line), IL18mut15 (Fuse 1111; open square, dashed line) and IL18mut16 (Fuse112; closed circle). The anti-HEL specific antibody (gray closed circle) was used as the negative isotype matched control and recombinant human IL-18 was added as a non-targeted reference (gray star). Based on the non-linear x-y plot of shown, the rank order of IL-18 induced IFNγ release was Fuse1116>Fuse1017>Fuse 1109>Fuse1111/Fuse1112>Fuse 1110. The PD-1 targeted activity of Fuse1116 and Fuse1017 were both greater than the human recombinant IL-18 reference, whereas Fuse1109. Fuse1112 and Fuse1112 were similar in activity to the human recombinant IL-18 reference and Fuse1110, based on the EC50_{IFNγ-release} was >30× weaker than the human recombinant IL-18 reference. A summary table of EC50s and maximum activity for the test articles are shown below the graph. For FIG. 29B, T cells were mixed with MDA-MB-231 engineered to express high cell surface levels of human ROR1 at an E:T of 5:1 in a titration of test articles in the presence of a suboptimal concentration of a ROR1 x CD3 bispecific antibody equivalent to 20% maximal activity (10 pM of Fuse608) and IFNγ released into the supernatant assessed after 72 hours. The test articles examined were the following IL-18 variants fused to an IgG1-LALA backbone targeted to PD-1 using the paratope from nivolumab: IL118mut2 (Fuse1017; reverse open triangle, dashed line), IL118mut9 containing a 10,000 fold attenuating mutant propeptide on the N-terminal of the mature cytokine (Fuse1044; closed reverse triangle), IL18mut2 containing a 10,000 fold attenuating mutant propeptide on the N-terminal of the mature cytokine (Fuse1042; closed square), IL18mut13 containing a 100 fold attenuating polypeptide on the N-terminal of the mature cytokine (Fuse1177; open circle, dashed line), and Fuse691 (nivolumab biosimilar; black closed circle). The anti-HEL specific antibody (gray closed circle) was used as the negative isotype matched control. Based on the non-linear x-y plot of shown, the rank order of IL-18 induced IFNγ release was Fuse1017>Fuse1044>Fuse1042/Fuse1177>>Fuse691. A summary table of EC50s and maximum activity for the test articles are shown below the graph.

### Example 15

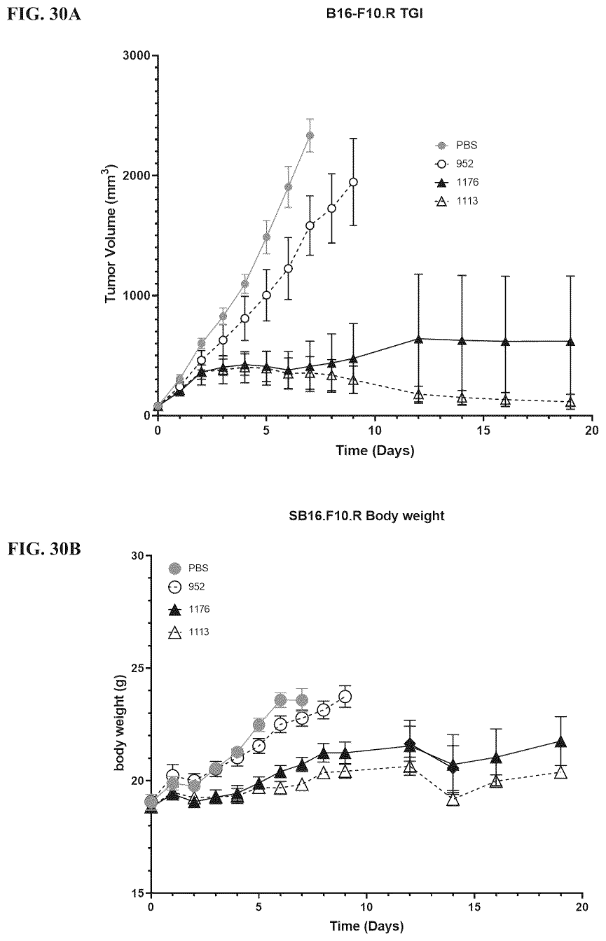

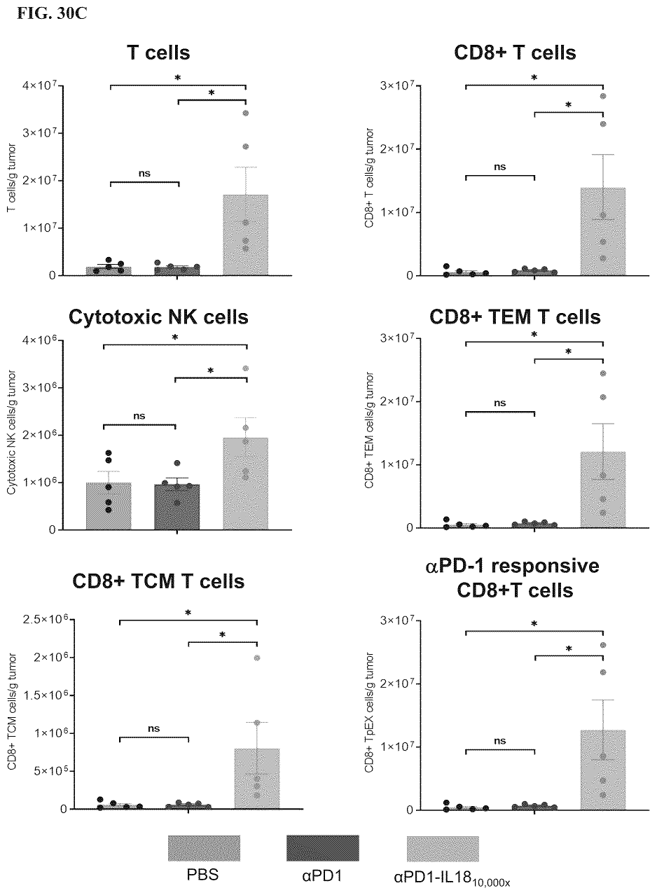

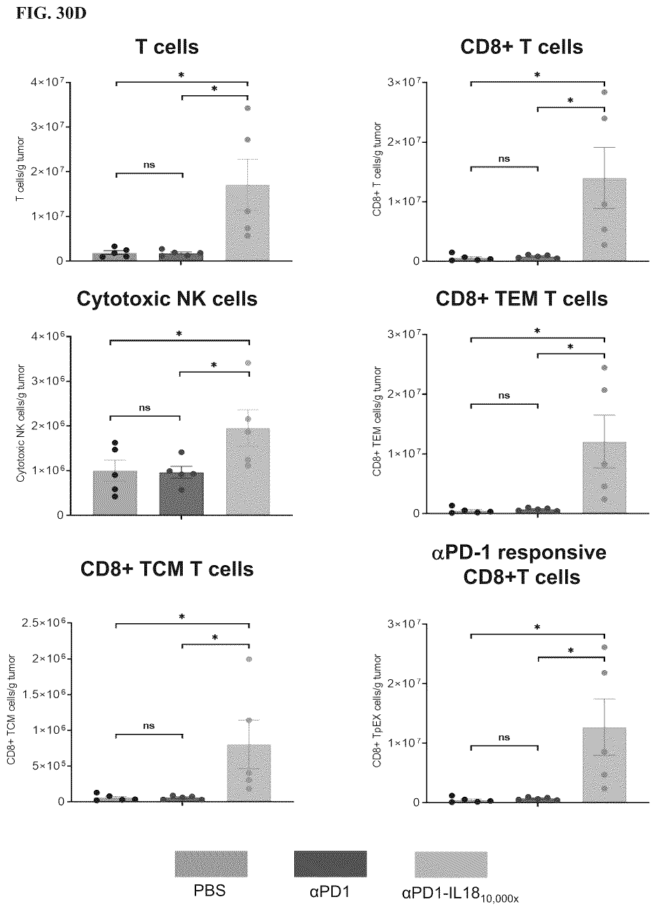

As shown in FIG. 30A-30B, we assessed the capacity of an exemplary IL-18 variant targeted to mouse PD-1 to mediate tumor growth inhibition of an aggressive syngeneic melanoma tumor in a fully immunocompetent mouse tumor model. C57BL/6 mice were subcutaneously injected with 1×10\hat{ }6 B16-F10 tumor cells into which we engineered human ROR1 (B16-F10-R) into the right flank. When the tumors reached 75 mm\hat{ }3, the mice were randomized into 4 groups of 5 mice each and treatment administered intraperitoneally on days 0, 3 and 6 at a dose of 15 mg/kg. The test articles examined were Fuse952 (open circle; recombinant anti-mouse-PD1 clone RMP1.14 human IgG1 LALA), Fuse 1113 (open triangle, dashed line; single arm RMP1.14 Fab x IL-18mut2A human IgG1 LALA in a knobs into holes configuration whereby the mouse PD1 specific Fab and IL-18 variant may be on the N-terminal knob and hole, respectively, or vice versa) and Fuse1176 (closed triangle; recombinant anti-mouse-PD1 clone RMP1.14 human IgG1 LALA in which IL-18mut2A has been fused to the N-terminus of the RMP1.14 light chain). PBS was used as the negative control treatment. FIG. 30A is x-y plot of tumor volume in mm\hat{ }3 versus time in days. FIG. 30B is x-y plot of body weight in grams versus time in days. Fuse1113 induced about 100% tumor growth inhibition (TGI) and a complete response rate of 60%. Fuse1176 induced about a 75% TGI and a 40% complete response rate. Transient weight loss was observed between days 10 and 15 in the groups receiving Fuse1113 and 1176. This was attributed to an ADA (anti-drug antibody) response, which is not unexpected for a fully human test article in a fully immunocompetent mouse model, which is xenogeneic relative to the test article.

On day 5 following the initiation of treatment, tumors were excised (n=5 mice per group), single cell suspensions generated and flow cytometry performed using the protocol described by Xu et al. (pubmed.ncbi.nlm.nih.gov/38091375/). As shown in FIG. 30C, the number of different lymphocyte subsets per grain of tumor was assessed from tumor bearing mice treated with PBS (light grey), anti-PD1 (black), and Fuse1113 (horizontal lines). TIL were defined as live mouse CD45+ cells. Within the TIL subset, T cells were defined as CD3+ cells, CD8+ T cells were defined as CD3+CD8+ cells, CD8+T effector memory cells (TEM; and effector CD8+ T cells) were defined as CD3+CD8+CD62L− CD44+ cells, CD8+T central memory cells (TCM) were defined as CD3+CD8+CD62L+CD44+ cells, and anti-PD-1 responsive CD8+ T cells (progenitor exhausted CD8+ T cells) were defined as CD3+CD8+PD−1+ TIM3− TCF-1+ cells. FIG. 30D depicts the frequency of total myeloid cells (CD11b+ cells), M2 macrophages (CD11b+F4−80+CD11c−CD206+ cells) and the ratios of CD8+ T cells to total Myeloid Derived Suppressor Cells (MDSC; CD11b+Ly6C+Ly6G− [M-MDSC] and CD11b+Ly6C-mid Ly6G+[G-MDSC]), CD8+ T cells to M2 macrophages, anti-PD-1 responsive CD8+ T cells to MDSC, and CD8+ T cells to CD4+ T cells. Relative to treatment with anti-PD-1 alone, treatment with Fuse1113 results in about a 10 fold increase in T cells, about a 20 fold increase in CD8+ T cells, about a 12 fold increase in CD8+ TEM cells, about a 13 fold increase in CD8+ TCM cells, and about a 20 fold increase in anti-PD-1 responsive CD8+ T cells. Total myeloid cells were decreased by about 50%, the ratio of CD8+ T cells to MDSC was increased by about 7 fold, CD8+ T cells to M2 macrophages was increased by about 14 fold, CD8+ T cells to CD4+ T cells was increased by about 6 fold and anti-PD-1 responsive CD8+ T cells to MDSC was increased by about 6 fold.

### Example 16

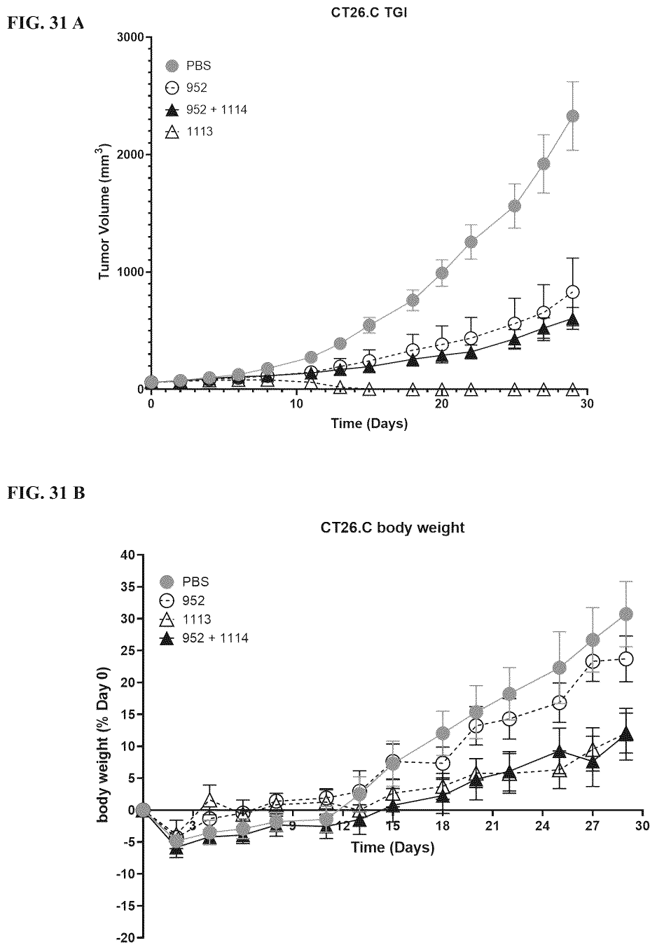

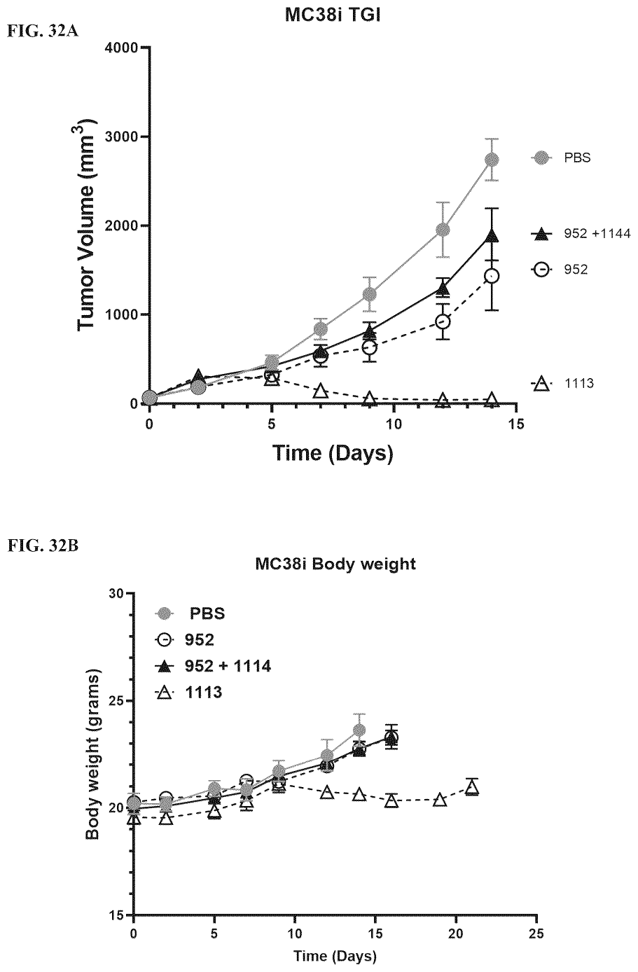

As shown in FIG. 31, we assessed the capacity of an exemplary IL-18 variant targeted to mouse PD-1 to mediate tumor growth inhibition in a syngeneic colorectal cancer tumor in a fully immunocompetent mouse tumor model. Balb/c mice were subcutaneously injected with 3×10\hat{ }6 CT26 tumor cells into which human Cadherin-17 was engineered (CT26-C) into the right flank. When the tumors reached 75-100 mm\hat{ }3, the mice were randomized into 4 groups of 5 mice each and treatment administered intraperitoneally on days 0, 4 and 8 at a dose of 15 mg/kg. The test articles examined were Fuse952 (open circle, dashed line; recombinant anti-mouse-PD1 clone RMP1.14 human IgG1 LALA), Fuse1113 (open triangle, dashed line; single arm RMP1.14 Fab x IL-18mut2A human IgG1 LALA in a knobs into holes configuration whereby the mouse PD1 specific Fab and IL-18 variant may be on the N-terminal knob and hole, respectively, or vice versa) and a combination of Fuse1114 and Fuse952 (closed triangle). Fuse 1114 is identical to Fuse1113 expect that it is targeted to human PD-1, which is not present in Balb/c mice and therefore served as the non-targeted Il-18mut2A control. PBS was used as the negative control treatment. FIG. 31A is x-y plot of tumor volume in mmA3 versus time in days. FIG. 31B is x-y plot of body weight in gramins versus time in days. Fuse 1113 induced about a complete response rate of 100%. The combination of Fuse1114 and Fuse952 was not appreciably different from Fuse952 alone. No appreciable weight loss was observed across the four groups of mice.

### Example 17

As shown in FIG. 32A-32B, we assessed the capacity of an exemplary IL-18 variant targeted to mouse PD-1 to mediate tumor growth inhibition in a syngeneic rapidly growing colorectal cancer tumor in a fully immunocompetent mouse tumor model. C57BL/6 mice were subcutaneously injected into the right flank with 1×10\hat{ }6 tumor cells of a rapidly growing clone of MC38 termed “MC38i”. This growth rate of MC38i is about double that of the widely used MC38 tumor model. It was not engineered. When the tumors reached 75-100 mm\hat{ }3, the mice were randomized into 4 groups of 5 mice each and treatment administered intraperitoneally on days 0, 4 and 8 at a dose of 15 mg/kg. The test articles examined were Fuse952 (open circle, dashed line; recombinant anti-mouse-PD1 clone RMP1.14 human IgG1 LALA), Fuse1113 (open triangle, dashed line: single arm RMP1.14 Fab x IL-18mut2A human IgG1 LALA in a knobs into holes configuration whereby the mouse PD1 specific Fab and IL-18 variant may be on the N-terminal knob and hole, respectively, or vice versa) and a combination of Fuse1114 and Fuse952 (closed triangle). Fuse1114 is identical to Fuse 1113 except that it is targeted to human PD-1, which is not present in Balb/c mice and therefore served as the non-targeted Il-18mut2A control. PBS was used as the negative control treatment. FIG. 32A is x-y plot of tumor volume in mm\hat{ }3 versus time in days. FIG. 32B is x-y plot of body weight in grams versus time in days. Fuse1113 induced about a complete response rate of 60%. The combination of Fuse1114 and Fuse952 was not appreciably different from Fuse952 alone. Transient weight loss was observed between days 7 and 15 in the group receiving Fuse1113. This was attributed to an ADA (anti-drug antibody) response, which is not unexpected for a fully human test article in a fully immunocompetent mouse model, which is xenogeneic relative to the test article.

### Example 18

We developed a first-in-class variant of IL-18 termed “HT18” fused to an anti-PD-1 antibody (αPD1-HT18cis). HT18 is 10,000-fold attenuated but retains nearly full activity of native cytokine when delivered to PD-1+ cells.

HT18 did not bind IL-18BP nor IL-18Rα in trans but can bind strongly to the IL-18Rα/β complex. Targeting HT18 to PD-1 augmented PD-1 induced T cell mediated proinflammatory cytokine release in-vitro.

Strong efficacy was observed with αPD1-HT18cis in multiple aggressive mouse tumor models including a complete response (CR) rate ranging from 60%-100% without weight loss or other signs/symptoms of toxicity

Immune memory and evidence supporting epitope spreading was observed in both mouse tumor models that induce both weak and strong emergency myelopoiesis

TGI was associated with about a 6-fold increase in TIL numbers relative to the PD-1 antibody benchmark with significant increases in CD8+ T cells, effector and central memory T cells, reduced tumor associated macrophages (TAM) and skewing towards a proinflammatory anti-tumor cellularity.

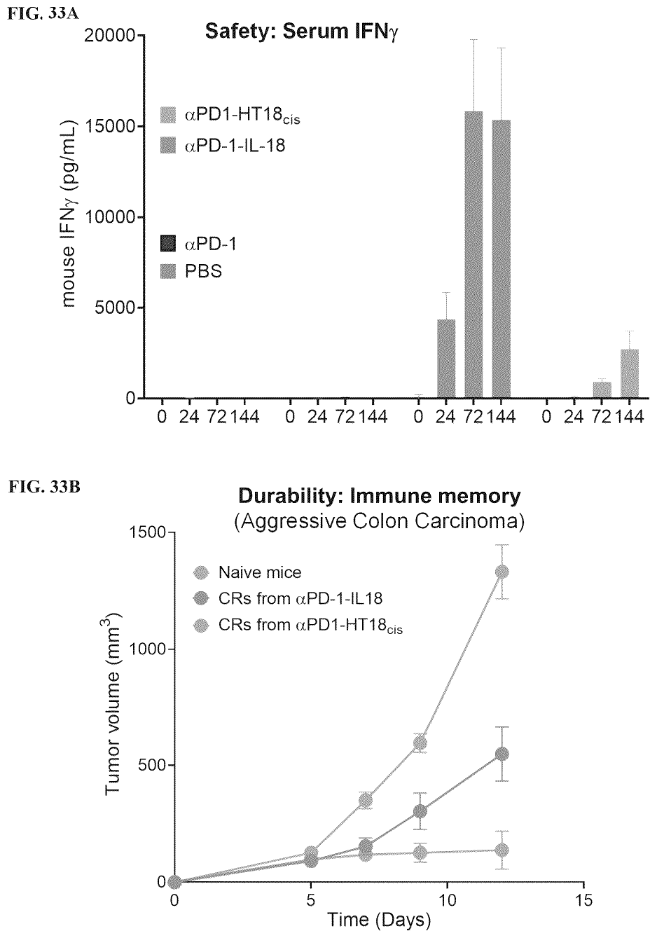

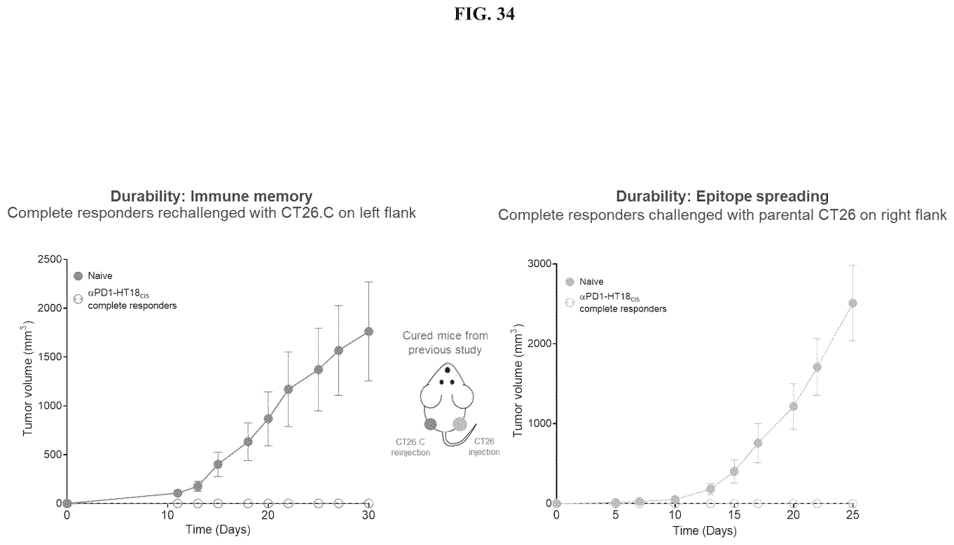

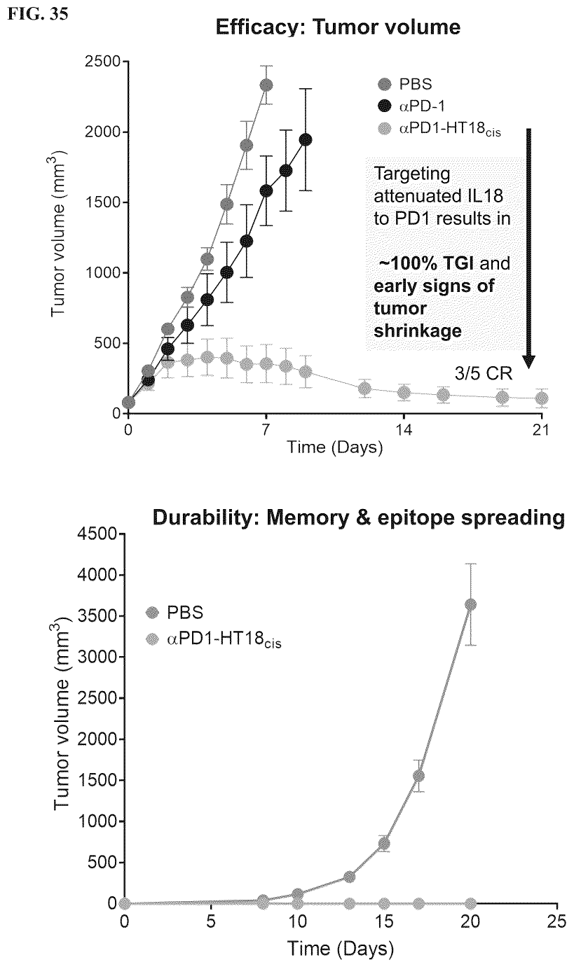

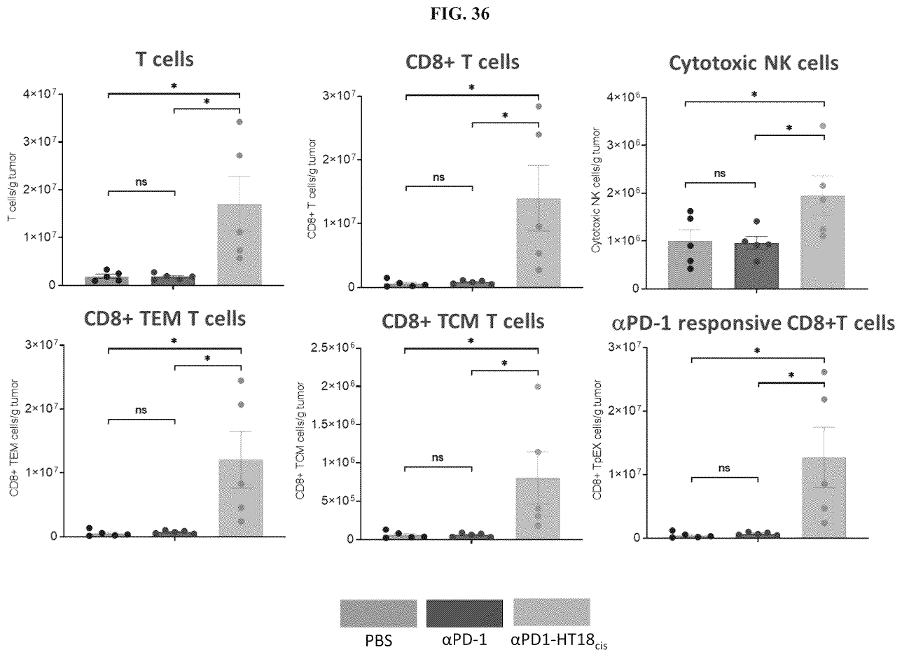

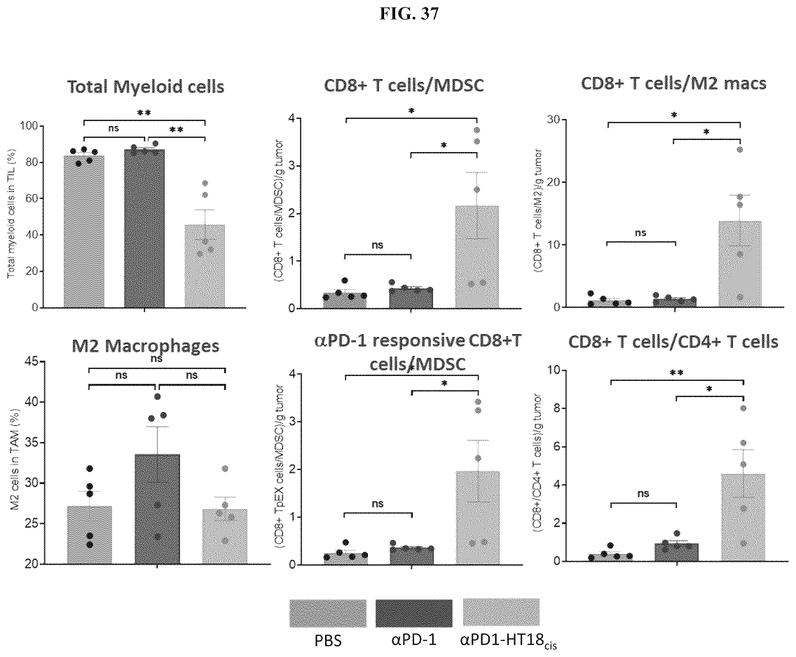

Some of these findings are shown in FIGS. 33-37.

### Example 19

### Human Peripheral Blood Mononuclear Cell (PBMC) Isolation

PBMCs were isolated from whole blood from healthy donors using Ficoll-Paque Plus medium. In brief, 35 mL of diluted whole blood (1 volume of whole blood vs 1 volume of PBS) was gently overlayed on top of 15 mL Ficoll-Paque Plus medium without disturbing the interface in a 50-mL conical tube. After centrifuging for 40 minutes at 400×g at room temperature without brake, the buffy coat (interface layer between Ficoll and serum) was collected and diluted in 5 volumes of PBS. After centrifuging for 5 minutes at 500×g at room temperature, PBMCs were resuspended in PBS and washed once in PBS by centrifuging for 5 minutes at 500×g at room temperature. PBMCs were then resuspended in 5 mL of ACK lysis buffer and incubated for 5 minutes at room temperature to remove red blood cell residues. After the 5-minute incubation period, 45 mL PBS was added to PBMCs and centrifuged for 15 minutes at 100×g at room temperature. At last, PBMCs were resuspended in culture medium (RPMI1640 with 10% heat-inactivated FBS and 1% penicillin/streptomycin) for cytotoxicity and IFN gamma release assay set-up. To store PBMCs for further usage, PBMCs were pelleted down by centrifuging for 5 minutes at 500×g at room temperature and then resuspended in freezing medium (90% heat-inactivated FBS and 10% dimethyl sulfoxide (DMSO)) for storage in liquid nitrogen.

### NK Cell Isolation and Expansion

Fresh PBMCs were washed twice in PBS and resuspended in EasySep Buffer from StemCell at a density of 50 million cells per mL in 50-mL conical tube. 50 μL of isolation cocktail from the NK cell isolation kit from StemCell was added to 1 mL of cell suspension and incubated at room temperature for 5 minutes. After the 5-minute incubation period, 50 μL of RapidSpheres from NK cell isolation kit was added to 1 mL of the cell/antibody cocktail mixture. The total volume of the cell/cocktail/bead mixture was brought up to 25 or 50 mL with EasySep Buffer and incubated at room temperature for 10 minutes on EASYSEP magnet. After the 10-minute incubation period, the solution was transferred to a new 50-mL tube by keeping the tube on the magnet and incubated at room temperature for 5 minutes on EASYSEP magnet. The solution with purified NK cell was collected. Freshly purified NK cells were used for expansion or frozen down and stored in liquid nitrogen for further usage.

To expand NK cells, 1×500 μL of ImmunoCult™ NK cell expansion coating material from ImmunoCult NK cell expansion kit from StemCell was added to a non-tissue culture-treated 24-well plate and incubated at room temperature for 2 hours. During the 2-hour incubation, freshly isolated NK cells were resuspended in ImmunoCult NK cell expansion medium from ImmunoCult NK cell expansion kit from StemCell at the density of 1 million per mL. After the 2-hour incubation, the plate was washed twice with PBS and 500 μL of NK cell suspension (0.5 million NK cells) was added to one well of the coated 24-well plate. The plate was incubated at 37° C. with 5% CO2 for 3 days and 500 μL of Immunocult NK cell expansion medium was added to the well with NK cells. The plate was further incubated for another 4 days. At day 7, NK cells were collected by centrifuge at 300×g for 10 minutes and resuspended in ImmunoCult NK cell expansion medium from ImmunoCult NK cell expansion kit from StemCell at the density of 0.2 million per mL. 1 mL of collected NK cells (0.2 million NK cells) was added to a well of new coated 24-well plate and incubated for 4 days at 37° C. with 5% CO2. At day 11, NK cells were collected again by centrifuge at 300×g for 10 minutes and resuspended in ImmunoCult NK cell expansion medium from ImmunoCult NK cell expansion kit from StemCell at the density of 0.2 million per mL. 1 mL of collected NK cells (0.2 million NK cells) was added to a well of new coated 24-well plate and incubated for another 3 days at 37° C. with 5% CO2. NK cells after 14-days' expansion were collected and frozen down and stored in liquid nitrogen for further usage.

### Cytotoxicity Assay

On the day before assay setting up, selective antibiotics were removed from target cell (MDA-MB-231 eGFP FLUC cells or T-47D eGFP FLUC cells). On the day of assay setting up, target cells were collected by brief TrypLE treatment and then washed with culture medium by centrifuging at 500×g for 5 minutes at room temperature. Target cell lines were then resuspended in culture medium to determine the cell viability by trypan blue exclusion on Cellometer. The viable cell density was adjusted to 50,000 cells/mL in culture media. 100 uL target cell suspension (5,000 target cell) was carefully dispensed to each well of a 96-well black clear flat-bottom tissue culture plate using multichannel pipettor. The plate was then incubated for 4-5 hours in tissue culture incubator to ensure that the target cells have attached to the bottom of the 96-well plate.

For the cytotoxicity assay using NK cells or PBMCs as effector cells. NK cells or PBMCs were pelleted down by centrifuge for 5 minutes at 500×g at room temperature and resuspended in culture medium. The viability of cells was also determined via trypan blue exclusion and the viable NK or PBMC density was adjusted to 3 million cells/mL in RPMI1640 medium with 10% FBS and 1% penicillin/streptomycin. After 4-5 hours of incubation, culture medium was carefully removed from 96-well plates with target cells. 50 μL of 3 million cells/mL NKs or PBMCs suspension (150,000 cells) was added to the designated well in the 96-well plate with target cell, which would result in the E:T ratio of 30:1. To result in the E:T ratio of 10:1, 5:1, 2:1 or 1:1, 3 million cells/mL of NKs or PBMCs were further diluted with RPMI1640 medium with 10% FBS and 1% penicillin/streptomycin by 3, 6, 15, or 30 times. 50 μL of diluted NK cells or PBMCs suspension was then added to the designated wells in the 96-well plate with target cell.

For the cytotoxicity assay using activated T cells as effector cells, isolated T cells were pretreated with human CD3/CD28 dynabeads (2 uL per million T cells) for 72 hours. After 72-hour activation. CD3/CD28 dynabeads were removed from activated T cells on magnet. Activated T cells were then pelleted down by centrifuge for 5 minutes at 500×g at room temperature and resuspended in culture medium. The viability of T cells was also determined via trypan blue exclusion and the viable T cell density was adjusted to 2 million cells/mL in RPMI1640 medium with 10% FBS and 1% penicillin/streptomycin. After 4-5 hours of incubation, culture medium was carefully removed from 96-well plates with target cells. 25 μL of 2 million cells/mL activated T cell suspension (50,000 cells) was added to the designated well in the 96-well plate with target cell, which would result in the E:T ratio of 10:1. Meanwhile, Fuse-608 was prepared in RPMI1640 medium with 10% FBS and 1% penicillin/streptomycin at the concentration of 40 pM. 25 μL of 40 pM prepared Fuse-608 was added to designated well in the 96-well plate with target cell and activated T cells.

FUSE proteins were prepared and serially diluted (5-fold serial dilution) in RPMI1640 medium with 10% FBS and 1% penicillin/streptomycin ranging from 200 nM to 2.56 pM. 50 μL of prepared FUSE proteins at different concentrations were then added to the designated wells in the 96-well plate with PBMC and target cells and incubated for the dedicated time period (24, 48 or 72 hours) at 37° C. with 5% CO2. At the end of the incubation period, the 96-well plates were centrifuged for 1 minute at 500×g to transfer 50 μL of supernatant to V-bottom storage plate using a multichannel pipettor for IFN gamma release, granzyme B release or HEK-BLUE-IL-18 cell activation assay. ONE-Glo luciferase Assay solution was brought to room temperature. 50 μL of One-Glo solution was then added to the designated well with cells and incubated for 2 minutes at room temperature. The bioluminescence (IFU) was measured on a plate reader with preset Bio-luminance protocol. Killing of target cell was calculated by using the formula [Killing percentage of testing sample=(IFU of sample with PBMC and target cell−IFU of testing sample)/(IFU of sample with PBMC and target cell−IFU of sample with PBMC alone)*100].

### T Cell Isolation

Fresh PBMCs were washed twice in PBS and resuspended in EasySep Buffer from StemCell at the density of 50 million cells per mL in 50-mL conical tube. 50 μL of isolation cocktail from T cell isolation kit from StemCell was added to 1 mL of cell suspension and incubated at room temperature for 5 minutes. After the 5-minutes' incubation, 50 μL of RapidSpheres from T cell isolation kit was added to 1 mL of the cell/antibody cocktail mixture. The total volume of the cell/cocktail/bead mixture was brought up to 25 or 50 mL with EasySep Buffer and incubated at room temperature for 10 minutes on EASYSEP magnet. After the 10-minutes' incubation, the solution was transferred to anew 50-mL tube by keeping the tube on the magnet and incubated at room temperature for 5 minutes on EASYSEP magnet. The solution with purified T cell was collected. Freshly purified T cells were frozen down and stored in liquid nitrogen for further usage.

### Dendritic Cell Generation

Fresh PBMCs were resuspended in AIM V medium at the density of 5 million per ml and 40 mL of PBMC suspension was added to a T-174 sterile cell culture flask. The flasks were incubated in a humidified incubator maintained at 37° C. and 5% CO2 for 2 hours. After 2 hours' incubation, the non-adherent cells were removed by firmly tapping the flask and completely aspirating off media in the flask. 40 mL of AIM-V medium was immediately added to the adherent cells with 800 U/ml GM-CSF and 500 U/ml IL-4 to each T-150 cell culture flask containing the adherent monocytes. The flasks were placed in a humidified incubator at 37° C. and 5% CO2 for 7 days. After the 7-day culture period, DCs were harvested, frozen down and stored in liquid nitrogen for further usage.

HEK-Blue-IL-18 Cell Activation Assay with Human Granzyme B-Cleaved FUSE Proteins or Supernatant from Cytotoxicity Assay

HEK-BLUE-IL-18 cells from Invivogen were maintained in culture medium (DMEM medium with 4.5 g/L glucose, 2 mM L-Glutamine, 10% (v/v) heat-inactivated fetal bovine serum (FBS), 100 U/mL penicillin, 100 μg/mL streptomycin, 100 μg/mL Normocin and 1×HEK-blue selection reagent). HEK-BLUE™ IL-18 cells are engineered from the human embryonic kidney 293 (HEK293) cell line to stably express genes encoding the IL-18 receptor (IL-18R) and IL-18 receptor accessory protein (IL-18RAP) and express an NF-κB/AP-1-inducible secreted embryonic alkaline phosphatase (SEAP) reporter gene, therefore being useful for detection of bioactive IL-18 by monitoring the activation of the NF-κB and AP-1 pathways via quantification in the supernatant of the SEAP level (which is produced upon activation of NF-κB) with a solution such as QUANTI-BLUE™ Solution. In addition, the responses to human TNF-α and IL-1β have been blocked in HEK-BLUE™ IL-18 cells, and so the HEK-BLUE™ IL-18 cells are responsive specifically to IL-18.

On the day of experiment setup. HEK-BLUE-IL-18 cells were gently rinsed twice with pre-wanned phosphate buffered saline (PBS) and then detached in PBS by tapping the flask. Detached HEK-Blue-IL-18 cells were resuspended in pre-warmed testing medium (DMEM with 4.5 g/L glucose, 2 mM L-Glutamine, 10% (v/v) heat-inactivated FBS, 100 U/mL penicillin, and 100 μg/mL streptomycin) at the density of 3×10⁵ cell per mL. To seed the cells (50,000 cells per well), 180 μL of resuspended HEK-Blue-IL-18 cells were added to designated wells in 96-well plate.

For FUSE protein cleavage with human granzyme B, human pro-granzyme B was activated with enterokinase (EK) by incubating 2 μg of human pro-granzyme B with 20 ng of EK in 1×PBS for 90 minutes at 37° C. To cleave FUSE protein, 20 μg of FUSE protein was incubated with 2 μg of EK-activated human granzyme B in assay buffer (50 mM HEPES (pH 7.4), 100 mM NaCl, 0.1% CHAPS, 1 mM EDTA, 10% Glycerol) for 10 minutes at 37° C. After the 10-minute incubation period, cleaved FUSE proteins were diluted in testing medium (DMEM with 4.5 g/L glucose, 2 mM L-Glutamine, 10% (v/v) heat-inactivated FBS, 100 U/mL penicillin, and 100 μg/mL streptomycin) from 12,500.000 μg/mL to 160 μg/mL (10× of final concentration) by 5-fold serial dilution. IL-18 was also diluted in testing medium from 4,000 μg/mL to 1.28 pg/mL (10× of final concentration) by 5-fold serial dilution. To treat HEK-Blue-IL-18 cells, 20 μL of prepared IL-18 or cleaved FUSE proteins were added to the designated wells with 50,000 cells in the 96-well plate and then gently mixed the cells with proteins. The 96-well plate was then incubated at 37° C. with 5% CO₂ for 24 hours.

To check the activity of the supernatant from cytotoxicity study in inducing the activation of HEK-Blue-IL-18 cell, 20 μL of supernatant from the cytotoxicity study was added to designated wells containing 50,000 cells in the 96-well plate and then gently mixed the cells with supernatant. The 96-well plate was then incubated at 37° C. with 5% CO2 for 24 hours.

After the 24-hour activation incubation period, 20 μL of HEK-Blue-IL-18 cell culture supernatant containing the secreted alkaline phosphatase was transferred to a new 96-well plate. Meanwhile, QUANTI-Blue solution was prepared by adding 1 mL of QB reagent and 1 mL of QB buffer to 98 mL of sterile water. 80 μL of the prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the absorbance at 630 nm using a spectrophotometer.

### T Cell and Dendritic Cell Co-Culture (MLR) Assay

Dendritic cells were generated from monocytes in PBMCs with 800 U/ml GM-CSF and 500 U/ml IL-4. Allogeneic T cells from PBMCs were mixed with dendritic cells at the ratio of 10:1 in RPMI1640 medium with 10% FBS and 1% penicillin/streptomycin. 100K T cells with 10K dendritic cells in 150 μL of medium were added to the wells in 96-well plate. Fuse proteins were prepared and serially diluted (5-fold serial dilution) in RPMI1640 medium with 10% FBS and 1% penicillin/streptomycin ranging from 200 nM to 2.56 pM. 150 μL of prepared FUSE proteins at different concentrations were then added to the designated wells in the 96-well plate with T cell and dendritic cells and incubated for the dedicated time period (48, 72, 96 or 144 hours) at 37° C. with 5% CO2. At the end of the incubation period, the 96-well plates were centrifuged for 1 minute at 500×g to transfer 250 μL of supernatant to V-bottom storage plate using a multichannel pipettor for IFN gamma release detection.

### IFN Gamma Release Detection

IFN gamma release from cytotoxicity assay was measured using human IFN gamma ELISA detection kit. In brief, coating antibody provided in the kit was diluted to suggested concentration by following the protocol provided by the kit manufacturer. 100 ul of diluted coating antibody was added to the Nunc MaxiSorp flat-bottom 96-well plate. Plates were sealed and incubated overnight at 4° C. On the following day, the plates were then washed 4 times with the Wash Buffer. To block non-specific binding and reduce background, 200 μL 1× Assay Diluent A was added, and the plates were incubated at room temperature for 1 hour on a plate shaker (400 rpm). After blocking, the plates were washed 4 times with Wash Buffer. 100 μL/well of standards (prepared with culture medium) or samples were then added to the appropriate wells and incubated at room temperature for 2 hours on a plate shaker (400 rpm). After the 2-hour incubation with samples or standards, the plates were washed 4 times with Wash Buffer and 100 μL of diluted Detection Antibody solution was added to incubate at room temperature for 1 hour on a plate shaker (400 rpm). After the 1-hour incubation period with detection antibody, the plates were washed 4 times with Wash Buffer and 100 μL of diluted Avidin-HRP solution was added to incubate at room temperature for 30 minutes on a plate shaker (400 rpm). After 30-minute incubation with Avidin-HRP, the plates were washed 5 times with Wash Buffer and 100 μL of freshly mixed TMB Substrate Solution was added to incubate at room temperature for 20 minutes in the dark. 100 μL of Stop Solution was then added to each well to stop the reaction. The absorbance at 450 nm was measured on a plate reader and the concentration of IFN gamma in each sample was back-calculated using the standard curve generated with the absorbance of different concentration of standards.

### Granzyme B Release Detection

Granzyme B release from cytotoxicity assay was measured using human granzyme B ELISA detection kit. In brief, coating antibody provided in the kit was diluted to suggested concentration by following the protocol provided by the kit manufacturer. 100 ul of diluted coating antibody was added to the Nunc MaxiSorp flat-bottom 96-well plate. The plates were sealed and incubated overnight at 4° C. The plates were then washed 3 times with the Wash Buffer. To block non-specific binding and reduce background, 200 μL 1× Assay Diluent A was added, and the plates were incubated at room temperature for 1 hour on a plate shaker (400 rpm). After blocking, the plates were washed 3 times with the Wash Buffer. 100 μL/well of standards (prepared with culture medium) or samples were then added to the appropriate wells and incubated at room temperature for 2 hours on a plate shaker (400 rpm). After the 2-hour incubation period with samples or standards, the plates were washed 3 times with Wash Buffer and 100 L of diluted Detection Antibody solution was added to incubate at room temperature for 1 hour on a plate shaker (400 rpm). After the 1-hour incubation period with detection antibody, the plates were washed 3 times with Wash Buffer and 100 μL of diluted Avidin-HRP solution was added to incubate at room temperature for 30 minutes on a plate shaker (400 rpm). After 30-minute incubation with Avidin-HRP, the plates were washed 4 times with Wash Buffer and 100 μL of freshly mixed TMB Substrate Solution was added to incubate at room temperature for 20 minutes in the dark. 100 μL of Stop Solution was then added to each well to stop the reaction. The absorbance at 450 nm was measured on a plate reader and the concentration of granzyme B in each sample was back-calculated by using the standard curve generated with the absorbance of different concentration of standards.

### Animal Models

C57BL/6 J and Balb/c mice (6-8 weeks old females) were housed under specific pathogen-free conditions and supplied with irradiated standard rodent chow and 0.2 μm filtered, autoclaved reverse osmosis water ad libitum. All the protocols and amendment(s) or procedures involving the care and use of animals were reviewed and approved by the Institutional Animal Care and Use Committee (IACUC) prior to conducting the studies. All animal experimental procedures were under sterile conditions at SPF (specific pathogen-free) facilities. C57BL/6 J were inoculated subcutaneously with either 1 million MC38i (a fast growing variant of MC38) or 1 million B16-F10-R (B16-F10 engineered to express hunan ROR1) into the flank of the mice. Balb/c mice were inoculated subcutaneously with 3 million CT-26c (CT-26 engineered to express human Cadherin-17) into the flank of the mice. Mice were randomly divided into treatment groups when tumor size reached 75-100. Mice were stratified based on the size of implanted tumor to ensure that tumor sizes were roughly equivalent between groups before the therapy. Each mouse was treated intraperitoneally PBS or test articles at the doses indicated in the examples section (generally 15 mg/kg) on days 0, 4 and 8 (MC38i and CT-26c) or days 0, 3 and 6 (B16-F10-R). Tumor volume and body weight was measured 2-3 times per week (MC38i and CT-26c) or daily (B16-F10-R) as required based on the growth rate of the tumor. Blood collection for pharmacokinetic and/or pharmacodynamic markers was collected and stored as serum for possible downstream evaluation on days 0, 1, 3, 6, and 10.

### Example 20

The N-terminal of pro-IL-18 was fused to the C-terminal of an IgG1 CH3 domain so as to engineer a variant of IL-18 expressible in mammalian cells. Pro-IL-18 was fused to the knob of a knob into hole heterodimeric IgG1 protein. Further modification was made to pro-IL-18 to reduce aggregation of the molecule such that each cysteine residue in both the pro-peptide and mature IL-18 was substituted with serine (“IL-18AS”, as in FUSE-480), alanine (“IL-18AA”, as in FUSE-481), or valine (“IL-18AV”, as in FUSE-442). Illustrations of the three variants are shown in panel A of FIG. 11.

Transient transfection in the ExpiCHO system resulted in titers of 191, 214, and 198 mg/L, for FUSE-480. FUSE-481, and FUSE-442, respectively. These Fc-pro-IL-18 fusion proteins were hypothesized as harboring a “masked” version of IL-18, where the biological activity of the fused IL-18 would be reduced until the propeptide is cleaved off. To evaluate cleavage of the propeptide, a cleavage site specific for the enterokinase (EK) was inserted within the propeptide (“pp”) upstream of mature IL-18 sequence in the location of the endogenous Caspase 1 site. EK was chosen because of its robust protease activity and activity in phosphate buffered saline. To evaluate the biological activity of the Fc-pro-IL-18 fusion proteins before and after treatment with EK, a reporter system was used. Here, HEK-Blue-IL-18 cells were used to quantify IL-18 activity. HEK-Blue IL-18 cells are made from HEK-293 engineered to express the human IL-18 receptor complex (IL-18Rα/β) and an NF-κb/AP-1-inducible secreted embryonic alkaline phosphatase (SEAP) reporter gene. The cells are also engineered not to respond to human TNF-α and IL-1β. Upon exposure to IL-18, HEK-Blue IL-18 produce SEAP in a dose dependent manner, which can be quantified via a colorimetric assay. We observed that compared to recombinant human mature IL-18, all three mutants induced lower biological activity greater than 1000 fold and up to 100,000 fold. Upon demasking (enzymatic cleavage) by treatment with EK, the biological activity of the released protein from the alanine-substituted mutant (FUSE-480) and the valine-substituted mutant (FUSE-442) was not substantially different from recombinant human mature IL-18. Following release of the serine mutant with EK (FIG. 11B), however, although biological activity was restored by >30,000 fold, it was ~50 fold less potent than recombinant human mature IL-18. Potency was measured as the concentration of test article that induced have maximal SEAP production (EC50-SEAP).

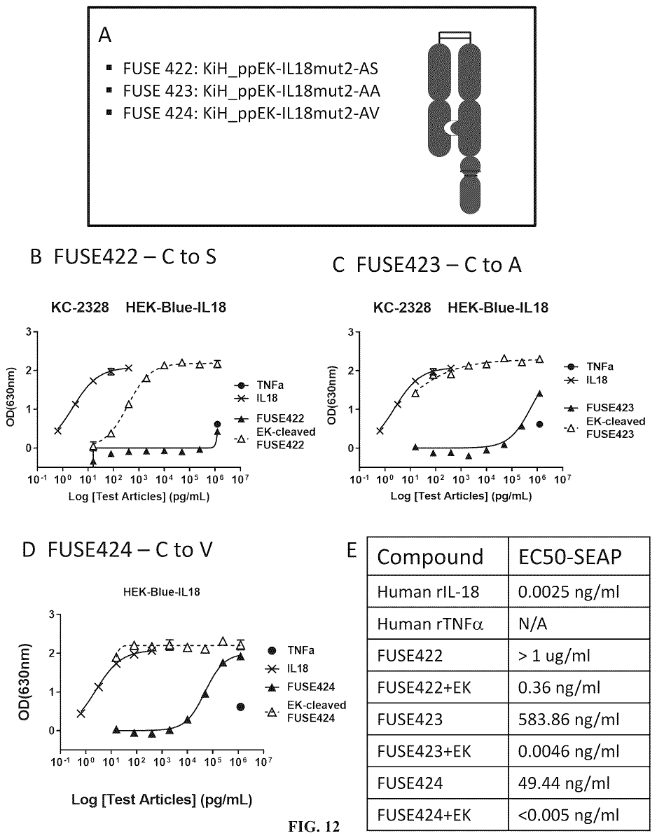

As shown in FIG. 12, pro-IL-18mut2 was fused to the knob of a knob into hole heterodimeric IgG1 protein. IL-18mut2 incorporates four amino acid substitutions hypothesized to reduce binding to IL-18BP while maintaining wild type binding to the IL-18 receptor complex. Further modification was made to pro-IL-18mut2 to reduce aggregation of the molecule such that each cysteine residue in both the pro-peptide and mature IL-18mut2 was substituted with serine (IL-18mut2AS), alanine (IL-18mut2AA), or valine (IL-18mut2AV). Illustrations of the three variants are shown in panel A of FIG. 12. As in FIG. 11, the biological activity was assessed using HEK-Blue. Compared to recombinant human mature IL-18, all three mutants induced lower biological activity of at least 100,000 fold. Upon demasking (enzymatic cleavage) by treatment with EK, the biological activity of the released protein from the alanine-substituted mutant (FUSE-423) and the valine-substituted mutant (FUSE-424) was not substantially different from recombinant human mature IL-18. Following release of the serine mutant with (FUSE422), however, although biological activity was restored by about 100,000 fold, it was about 100 fold less potent than recombinant human mature IL-18. Potency was measured as the concentration of test article that induced have maximal SEAP production (EC50-SEAP).

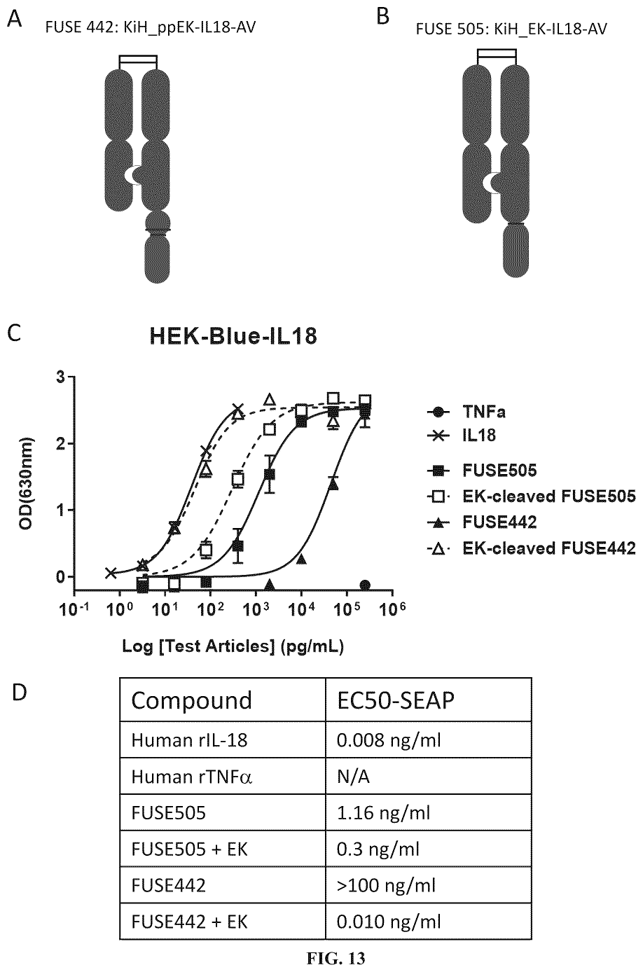

As shown in FIG. 13, we examined the impact of a propeptide on masking of IL-18AV biological activity in the context of Fc fusions variants incorporating IL-18AV on the C-terminal of the Fc with (panel A, FUSE-442; Fc-EKpp-IL-18AV) or without (panel B, FUSE-505; Fc-EK-IL-18AV) the propeptide. For the fusion in which the propeptide was removed, the EK cleavage site that replaced the Caspase 1 site was moved to a position directly in between the CH2 domain of the knob and mature IL-18AV without the addition of a flexible linker. Using the HEK-Blue IL-18AV reporter cell assay as a readout, we exposed these cells to a titration of FUSE-442 or FUSE-505 with or without treatment with EK. The biological activity, measured as the EC50-SEAP, of FUSE-442 and -505 were attenuated compared to human recombinant IL-18AV at the orders of ~150 fold and ~15,000 fold, respectively (panel C) such that incorporation of the propeptide contributed to ~100-fold additional attenuation compared to the Fc fusion alone. Treatment with EK, designed to cleave off the IL-18AV fragment from FUSE-505 and FUSE-442 led to restoration of biological activity. In the case of FUSE-442, treatment with EK resulted in biological activity that was not substantially different from recombinant human mature IL-18AV. For FUSE-505, however, although biological activity was restored following treatment with EK, potency remained reduced by ~30 fold compared to recombinant human mature IL-18AV. The EC50-SEAP for each compound is shown in panel D. Given that FUSE-505 and FUSE-442 both harbor the same IL-18AV, the difference in activity after demasking with EK could not be explained by any difference in IL-18AV variant. Rather, we observed that EK was far less efficient at releasing IL-18AV from FUSE-505 (<10% cleavage efficiency) than FUSE-4442 (>95% cleavage efficiency). Hence, the reduced biological activity observed from EK cleaved FUSE-505 versus EK cleaved 442 was the result of less IL-18AV being released by the former compared to the latter. We hypothesize that the lack of a flexible linker between the CH3 domain of the knob and IL-18AV in FUSE-505 resulted in a mostly inaccessible EK cleavage site. In contrast, the propeptide incorporated into FUSE-442 allowed for sufficient access of EK to its cleavage site for efficient release of IL-18AV. The data indicates strongly that the in the context of an IL-18AV Fc fusion protein, the propeptide is not required but contributes to masking of IL-18AV biological activity. In the absence of the propeptide, masking is likely the consequence of steric hindrance mediated by the protein fused to the N-terminal of IL-18AV

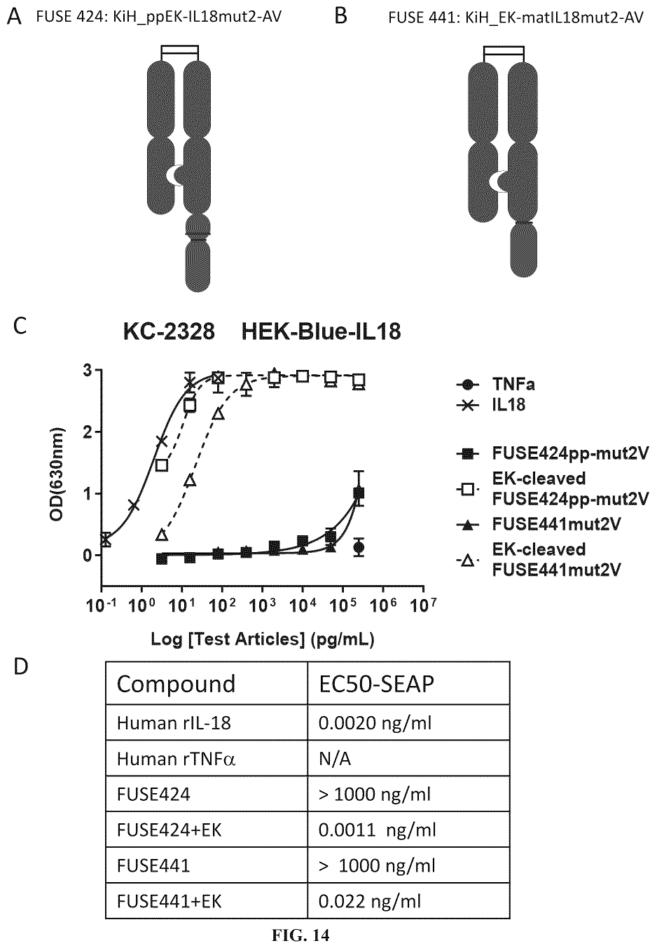

As shown in FIG. 14, as in FIG. 13 with IL-18AV, we examined the impact of a propeptide on masking of IL-18mut2AV biological activity in the context of Fc fusions variants incorporating IL-18AVmut2 on the C-terminal of the Fc with (panel A of FIG. 14, FUSE-424; Fc-EKpp-IL-18mut2AV) or without (panel B, FUSE-441; Fc-EK-IL-18mut2AV) the propeptide. The biological activity of FUSE-441 and FUSE424 were highly attenuated compared to human recombinant IL-18 at the orders of >100,000 fold (panel C). Treatment with EK led to restoration of biological activity. In the case of FUSE-424, treatment with EK resulted in biological activity that was not substantially different from recombinant human mature IL-18. For FUSE-441, however, although the vast majority of biological activity was restored following treatment with EK, potency remained reduced by ~10 fold compared to recombinant human mature IL-18. The EC50-SEAP for each compound is shown in panel D. Given that FUSE-441 and FUSE-442 both harbor the same IL-18mut2AV, the difference in activity after demasking with EK could not be explained by any difference in IL-18 variant. Rather, we observed that EK was far less efficient at releasing IL-18mut2AV from FUSE-441 (<20% cleavage efficiency) than FUSE-424 (>95% cleavage efficiency). Hence, the reduced biological activity observed from EK cleaved FUSE-441 versus EK cleaved FUSE-424 was likely the result of less IL-18mut2AV being released by the former compared to the latter. We hypothesize that the lack of a flexible linker between the CH3 domain of the knob and IL-18mut2AV in FUSE-441 resulted in a mostly inaccessible EK cleavage site. For example, a flexible linker can be the propeptide; or the propeptide behaves as a flexible linker. In contrast, the propeptide incorporated into FUSE-424 allowed for sufficient access of EK to its cleavage site for efficient release of IL-18mut2AV. The data indicates strongly that the in the context of an IL-18mut2AV Fc fusion protein, the propeptide is not required for masking of IL-18mut2AV biological activity but is rather the consequence of steric hindrance mediated by the protein fused to the N-terminal of IL-18mut2AV. As such, we believe that any N-terminal protein or polypeptide of sufficient size (e.g., about 4 kDa or larger, e.g., the propeptide is about 4 kDa, Fc is about 28 kDa as a monomer, HSA is about 66 kDa, VHH is about 14 kDa) would be capable of masking the biological activity of IL-18mut2AV; and that an EK cleavage site containing linker of sufficient size (e.g., 25 amino acid or longer) to allow access to EK, when incorporated between the Fc or other N-terminal protein mask and IL-18mut2AV, would substitute for the EK containing propeptide. Further, should such N-terminal masking protein or fragment thereof be engineered or naturally be transported through the Endoplasmic Reticulum (ER), this would result in expression yields from transient transfection of mammalian cells such as Expi-CHO acceptable for therapeutic development.

Additional examples of N-terminal protein or polypeptide of sufficient size to mask the activity of IL-18 variants include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A, E or S and n=1-5 or in some embodiments, an integer larger than 5, (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG)ₙ wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5. As further examples, N-terminal protein or polypeptide of sufficient size to mask the activity of IL-18 variants include but are not limited to a short polypeptide of 2, 3, or 4 amino acids in length, or flexible linkers or amino acid chain of 2-5, 6-10, 11-15, 16-20, 21-25, 26-30 amino acids. As further examples, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids.

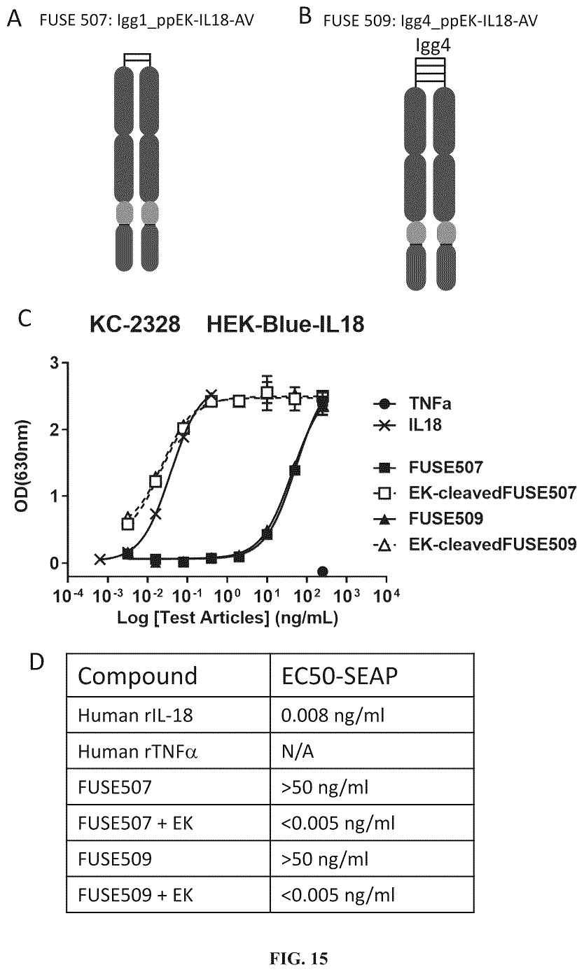

As shown in FIG. 15, we examined the impact of fusing the propeptide-IL-18AV fusion to the C-terminus of wild type IgG1 (FUSE-507, panel A) and wild type IgG4 (FUSE-509, panel B). In both formats, one molecule of the propeptide-IL-18AV fusion are linked to each Fc-CH3 domain, resulting in two molecules of the propeptide-IL-18AV fusion per IgG1 or IgG4 homodimer. As previously described, a cleavage site specific for EK was inserted between the propeptide and mature IL-18 sequence in the location of the endogenous Caspase-I site. To evaluate the biological activity of the Fc-pro-IL-18 fusion proteins before and after treatment with EK, the HEK-Blue-IL-18 cell reporter system was used. Using this systems as a readout, we exposed HEK-Blue-IL-18 cells to a titration of FUSE-507 (IgG1Fc-EKpp-IL-18AV) or FUSE-509 (IgG4Fc-EKpp-IL-18AV) with or without treatment with EK. The biological activity, measured as the EC50-SEAP, of FUSE-507 and -509 were highly attenuated compared to human recombinant IL-18 at the orders of >10,000 fold (panel C). Treatment with EK, designed to cleave off the IL-18AV fragment from FUSE-507 and FUSE-509 led to restoration of biological activity that was approximately 2 fold greater than hrIL-18. This difference was likely the result of two molecules of IL-18AV being release per IgG1 or IgG4 fusion resulting in about a 2:1 molar ratio of IL-18AV to hrIL-18 when the IgG1 or IgG4 fusion proteins were fully cleaved by EK. The EC50-SEAP for each compound is shown in panel D. We also generated versions of FUSE-507 and FUSE-509 without the propeptide; IgG1Fc-EK-IL-18AV and IgG4Fc-EK-IL-18AV, respectively. These did not express well, likely due to the propensity of IL-18 to form homodimers and the absence of a flexible linker between the CH3 domain of IgG1 or IgG4 and mature IL-18AV (data not shown). The data indicate that the in the context of a propeptide-IL-182AV fusion, it is possible to obtain both expression exceeding 135 mg/L and masking/attenuation of IL-18AV biological activity as high or greater than 100,000 fold (compare FIG. 15 to FIGS. 11 and 13) whether IL-18AV is fused to the C-terminus of wild type IgG1 or IgG4 (two molecules of IL-18AV) or an IgG1 knob into hole heterodimer (one molecule of IL-18AV). The formats utilizing wild type IgG1 or IgG4 indicate allowance for straightforward plug and play fusion of propeptide-IL-18 fusions, their variants and fragments thereof to an array of monoclonal antibodies including commercialized ones such as Avelumab (anti-PDL1), Cetuximab (anti-EGFR), Trastuzumab (anti-HER2/neu), etc.

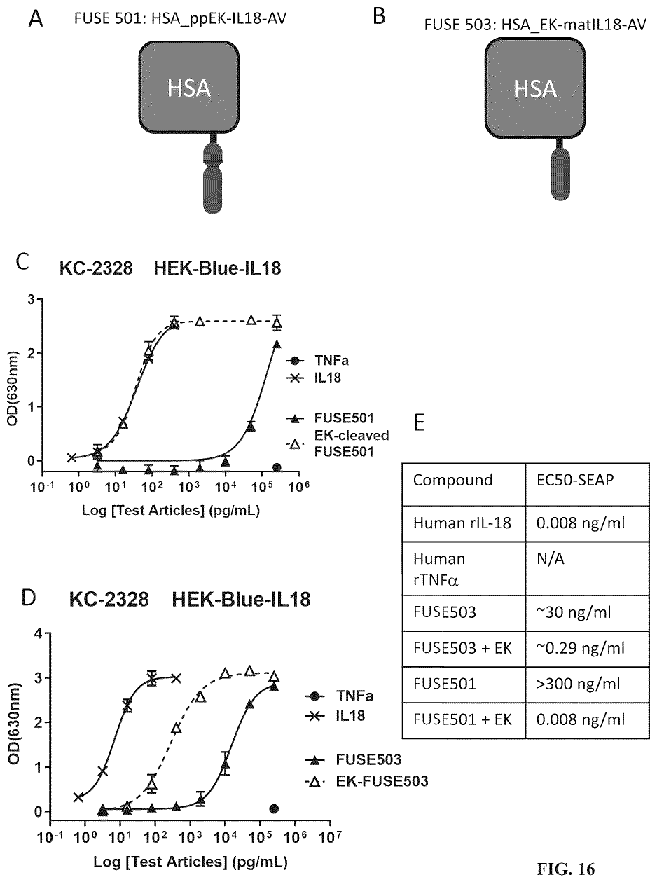

As shown in FIG. 16, to address the universality of masking of IL-18 and its variants by a polypeptide fused to the N-terminus of IL-18's mature form, we examined the capacity of an N-terminal protein (mask) distinct from the Fc domain of IgG to attenuate the biological activity of IL-18-AV with or without the propeptide. The N-terminus of IL-18AV with (panel A, FUSE-501) or without (panel B, FUSE-503) the propeptide was fused to the C-terminus of Human Serum Albumin (HSA). For the fusion in which the propeptide was removed, the EK cleavage site that replaced the Caspase 1 site was moved to a position directly in between the C-terminus of HSA and mature IL-18AV without the addition of a flexible linker. Using the HEK-Blue IL-18 reporter cell assay as a readout, we exposed these cells to a titration of FUSE-501 (HSA-EKpp-IL-18AV) or FUSE-503 (HSA-EK-IL-18AV) with or without treatment with EK. The biological activity, measured as the EC50-SEAP of FUSE-501 (panel C) was highly attenuated compared to human recombinant IL-18 at the order of ~35,000 fold. FUSE-503 (panel D), which does not contain the propeptide, was also attenuated at the order of ~3,500 fold. Treatment with EK, designed to cleave off the IL-18AV fragment from FUSE-501 and FUSE-503 led to restoration of biological activity. In the case of FUSE-501, treatment with EK resulted in biological activity that was not substantially different from recombinant human mature IL-18. For FUSE-503, however, although biological activity was increased by treatment with EK it remained ~40 fold weaker than human recombinant IL-18. The EC50-SEAP for each compound is shown in panel E. Given that FUSE-501 and FUSE-503 both harbor the same IL-18AV, the difference in activity after demasking with EK could not be explained by any difference in IL-18 variant. Rather, we observed that EK was far less efficient at releasing IL-18AV from FUSE-503 (<20% cleavage efficiency) than FUSE-501 (>95% cleavage efficiency). Hence, the reduced biological activity observed from EK cleaved FUSE-503 versus EK cleaved -501 was the result of less IL-18AV being released by the former compared to the latter. We hypothesize that the lack of a flexible linker between the C-terminus of HSA and IL-18AV in FUSE-503 resulted in a mostly inaccessible EK cleavage site. In contrast, the propeptide incorporated into FUSE-501 allowed for sufficient access of EK to its cleavage site for efficient release of IL-18AV. Taken together with data obtained from the Fc fusions, the results indicate strongly that the in the context of an HSA-IL-18AV fusion protein, the propeptide is not required for masking of IL-18AV biological activity but may contribute to greater attenuation when present in HSA-IL-18 fusion proteins. For both FUSE-501 and -503, masking appears to be the consequence of steric hindrance mediated by the protein fused to the N-terminal of IL-18AV. As such, this data provides further support that any N-terminal protein of sufficient size would be capable of masking the biological activity of IL-18AV and that an EK cleavage site containing linker of sufficient size to allow access to EK, when incorporated between the Fc, HSA or other N-terminal protein mask and IL-18AV, would substitute for the EK containing propeptide. Further, should such N-terminal masking protein or fragment thereof be engineered or naturally be transported through the Endoplasmic Reticulum (ER), this would result in expression yields from transient transfection of mammalian cells such as Expi-CHO acceptable for therapeutic development.

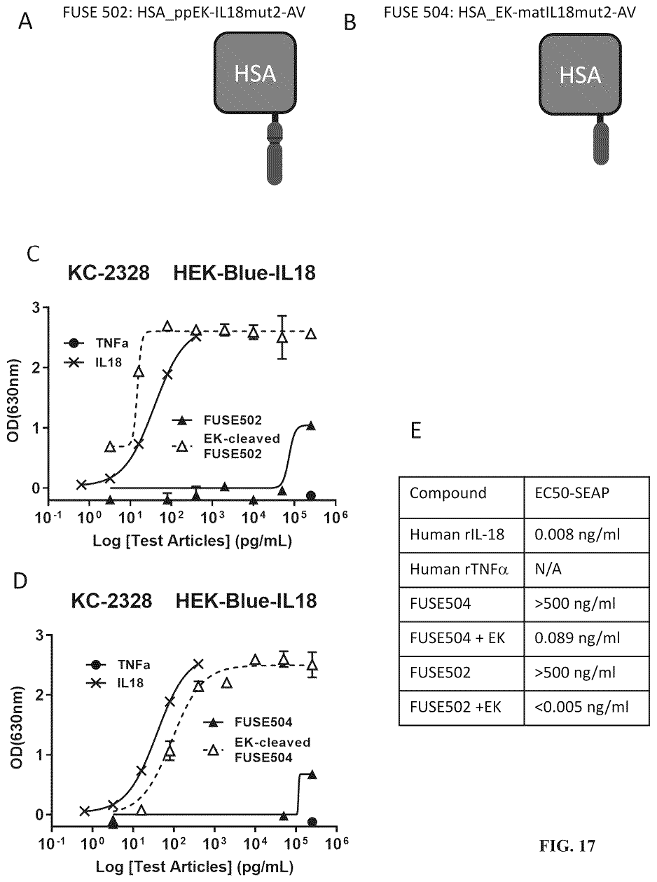

As shown in FIG. 17, to address the universality of masking of IL-18 and its variants by a polypeptide fused to the N-terminus of IL-18's mature form, we examined the capacity of an N-terminal protein (mask) distinct from the Fc domain of IgG to attenuate the biological activity of IL-18-mut2AV with or without the propeptide. The N-terminus of IL-18mut2AV with (panel A, FUSE-502) or without (panel B, FUSE-504) the propeptide was fused to the C-terminus of Human Serum Albumin (HSA). For the fusion in which the propeptide was removed, the EK cleavage site that replaced the Caspase 1 site was moved to a position directly in between the C-terminus of HSA and mature IL-18AV without the addition of a flexible linker. Using the HEK-Blue IL-18 reporter cell assay as a readout, we exposed these cells to a titration of FUSE-502 (HSA-EKpp-IL-18mu2AV) or FUSE-504 (HSA-EK-IL-18mut2AV) with or without treatment with EK. The biological activity, measured as the EC50-SEAP, of FUSE-502 (panel C) and FUSE-504 (panel D) were highly attenuated compared to human recombinant IL-18 at the orders of at least ~30,000 fold and ~50,000 fold, respectively. Treatment with EK, designed to cleave off the IL-18mut2AV fragment from FUSE-504 and FUSE-502 led to restoration of biological activity. In the case of FUSE-502, treatment with EK resulted in biological activity that was not substantially different from recombinant human mature IL-18 (panel C). For FUSE-504, although the vast majority of biological activity was restored following treatment with EK, potency remained reduced by ~3 fold compared to recombinant human mature IL-18 (panel D). The EC50-SEAP for each compound is shown in panel E. Given that FUSE-504 and FUSE-502 both harbor the same IL-18mut2AV, the difference in activity after demasking with EK could not be explained by any difference in IL-18 variant. Rather, we observed that EK was far less efficient at releasing IL-18mut2AV from FUSE-504 (<20% cleavage efficiency) than FUSE-502 (>95% cleavage efficiency). Hence, the reduced biological activity observed from EK cleaved FUSE-504 versus EK cleaved -502 was the result of less IL-18mut2AV being released by the former compared to the latter. We hypothesize that the lack of a flexible linker between the C-terminus of HAS and IL-18mut2AV in FUSE-504 resulted in a mostly inaccessible EK cleavage site. In contrast, the propeptide incorporated into FUSE-502 allowed for sufficient access of EK to its cleavage site for efficient release of IL-18mut2AV. Taken together with data obtained from the Fc fusions, the results indicate strongly that in the context of an HSA-IL-18mut2AV fusion protein, the propeptide is not required for masking of IL-18mut2AV biological activity but is rather the consequence of steric hindrance mediated by the protein fused to the N-terminal of IL-18mut2AV. As such, this data provides farther support that any N-terminal protein of sufficient size would be capable of masking the biological activity of IL-18mut2AV and that an EK cleavage site containing linker of sufficient size to allow access to EK, when incorporated between the Fc, HAS or other N-terminal protein mask and IL-18mut2AV, would substitute for the EK containing propeptide. Further, should such N-terminal masking protein or fragment thereof be engineered or naturally be transported through the Endoplasmic Reticulum (ER), this would result in expression yields from transient transfection of mammalian cells such as Expi-CHO acceptable for therapeutic development.

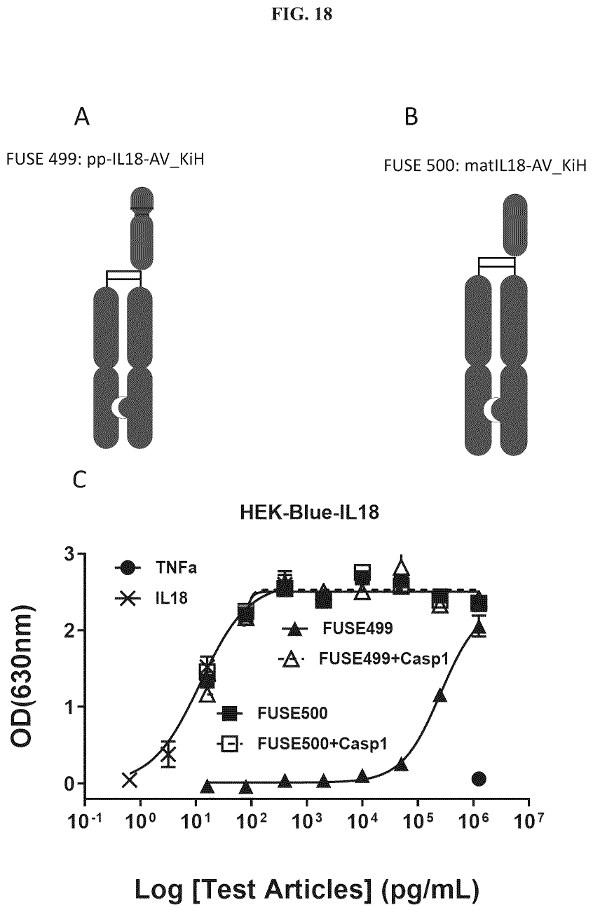

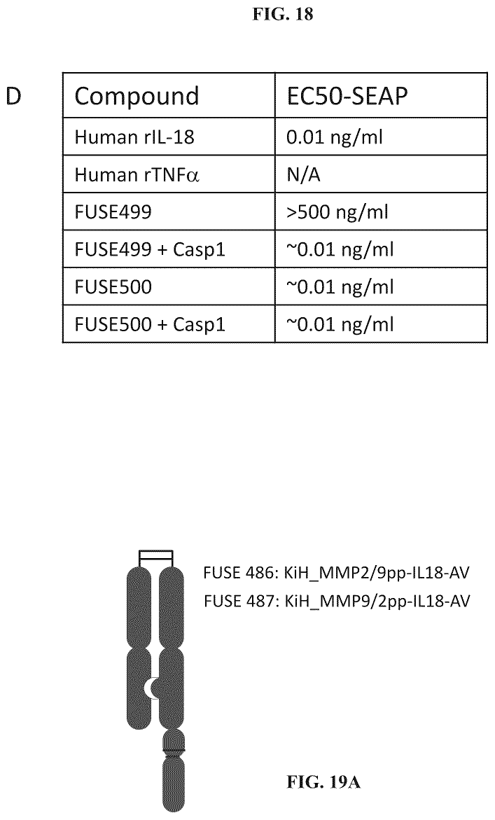

As shown in FIG. 18, we examined the impact of the propeptide on the biological activity of a single IL-18AV fused to the N-terminus of IgG1 Fc. Panels A and B are exemplary illustrations of IL-18AV fused on the knob of a knob into hole IgG1-Fc domain with the propeptide (FUSE-499: Fc-pp-IL-18AV; panel A) or without the propeptide (FUSE-500; Fc-IL-18AV; panel B). In this case, we used a wild type propeptide in which all cysteine residues were substituted for valine but the Caspase 1 site was maintained. To allow for translocation of both constructs into the Endoplasmic Reticulum (ER) of mammalian cells and hence expression/secretion, the signal peptide from the Ig Kappa chain (IgK leader) was encoded upstream of either the propeptide of FUSE-499 or the IL-18AV of FUSE-500. Using the HEK-Blue IL-18 reporter cell assay as a readout, we examined the capacity of our C→V (AV) propeptide to mask IL-18AV (FUSE-499) and the ability of Caspase 1 to demask/restore biological activity. The biological activity, measured as the EC50-SEAP, of FUSE499 was highly attenuated compared to human recombinant IL-18 at the order of >50,000 fold (panel C). In contrast, in the absence of the propeptide in FUSE-500, there was no reduction in biological activity compared to human recombinant IL-18, indicating that in the absence of a the propeptide (or another polypeptide) attached to the N-terminus of mature IL-18, the IL-18-Fc fusion protein is fully functional. Importantly, de-masking of FUSE-499 with Caspase I resulted in restoration of biological activity that was not appreciably different from to human recombinant IL-18. Treatment of FUSE-500, which did not contain a masking domain nor Caspase 1 cleavage site, with Caspase 1 served as a negative control and indeed had no effect on biological activity. This data indicates that in the configuration whereby IL-18AV is linked to the N-terminus of an IgG, the propeptide is required for attenuation. Taken together with our previous data that a CH3 domain or HSA attenuated the biological activity of IL-18AV and IL-18mut2AV when fused to the N-terminus of each IL-18 variant without the presence of a propeptide, we believe that any polypeptide of sufficient size fused to the N-terminal of mature IL-18 and/or its variants and fragments thereof would be capable of masking the biological activity of IL-18. While the smallest polypeptide tested was the propeptide (~6 kDa), polypeptides as small as 2 kDa would also be of sufficient size. In various embodiments, the short polypeptide or protein being about 2 kDa or no greater than 250 kDa. As additional examples, the short polypeptide or protein is about 2-5 kDa, about 6-10 kDa, about 11-20 kDa, about 21-30 kDa, about 31-40 kDa, about 41-50 kDa, about 51-75 kDa, about 76-100 kDa, about 101-125 kDa, about 126-150 kDa, about 151-175 kDa, about 176-200 kDa, about 201-225 kDa, or about 256-250 kDa. In various embodiments, the short polypeptide is 1, 2, 3, or 4 amino acids, or flexible linkers or amino acid chain of 5, 6, 7, 8, 9, 10, 11, 12, 13, 14, 15, 16, 17, 18, 19, 20, 25, 30, or 35 amino acids. Additional examples small polypeptides capable of masking the biological activity of IL-18 and IL-18 variants include but are not limited to a dimer of two amino acids, a tri-mer of three amino acids, a 4-mer of four amino acids, a 5-mer of five amino acids, or a peptide selected from the group consisting of T. PT. MPT. S, GS, GGS, GGGS (SEQ ID NO:235), and (GGGGX_{λ} (SEQ ID NO:236))ₙ wherein X_{λ} is Q, A, E or S and n=1-5 or an integer larger than 5. In some embodiments, the amino acid linker has the amino acid sequence of (GGGGS (SEQ ID NO:237))n where n is an integer between 1 and 5, thereby an amino acid linker of 25 amino acids or shorter in length. Additional examples include (X_{λ}GGGG (SEQ ID NO:317))ₙ wherein X_{λ} is Q, A, E or S and n=1-5 or in some embodiments, an integer larger than 5, (GX_{λ}GGG (SEQ ID NO:318))ₙ wherein X_{λ} is Q, A, E or S and n=1-5 or in some embodiments, an integer larger than 5, (GGX_{λ}GG (SEQ ID NO:319))ₙ wherein X_{λ} is Q, A. E or S and n=1-5 or in some embodiments, an integer larger than 5. (GGGX_{λ}G (SEQ ID NO:320))ₙ wherein X_{λ} is Q, A, E or S and n=1-5 or in some embodiments, an integer larger than 5. Still additional examples include (X_{λ}GGG)ₙ wherein X_{λ} is Q, A, E or S and n=1-5 or in some embodiments, an integer larger than 5, (GX_{λ}GG)ₙ wherein X_{λ} is Q, A, E or S and n=1-5 or in some embodiments, an integer larger than 5, (GGX_{λ}G)ₙ wherein X_{λ} is Q, A, E or S and n=1-5 or in some embodiments, an integer larger than 5, (GGGX_{λ})ₙ wherein X_{λ} is Q, A, E or S and n=1-5 or in some embodiments, an integer larger than 5. Still additional examples include (X_{λ}GG)ₙ wherein X_{λ} is Q, A, E or S and n=1-5 or in some embodiments, an integer larger than 5, (GX_{λ}G)ₙ wherein X_{λ} is Q, A, E or S and n=1-5 or in some embodiments, an integer larger than 5, (GGX_{λ})ₙ wherein X_{λ} is Q, A, E or S and n=1-5 or in some embodiments. Still additional examples include (X_{λ}G)ₙ wherein X_{λ} is Q, A, E or S and n=1-5 or in some embodiments, an integer larger than 5, (GX_{λ})ₙ wherein X_{λ} is Q, A, E or S and n=1-5 or in some embodiments, an integer larger than 5.

Further, the propeptide in FUSE-499 is not naturally transported through the ER but was engineered to do so via addition of an IgK leader sequence upstream to it. This indicates that any polypeptide of sufficient size fused to the N-terminus of mature IL-18 and/or its variants and fragments thereof would be expected to attenuate the biological activity of IL-18. That is, the N-terminal polypeptide might naturally translocate into the Endoplasmic Reticulum (ER) or it may be engineered to do so. In both cases, transport through the ER is important for obtaining expression yields from transient transfection of mammalian cells such as Expi-CHO acceptable for therapeutic development.

As shown in FIGS. 19A-19I, we examined whether proteases other than the proof of concept EK could be used to demask/activate pro-IL-18. As such, we chose (a) the matrix metalloproteinase (MMP), MMP2, reported to be preferentially over-expressed in the tumor microenviromnent and (b) Granzyme B, which is released by cytotoxic lymphocytes including NK cells and CD8⁺ T cells and may therefore accumulate in inflamed tumors. For MMP2, we replaced the EK cleavage site within the propeptide (pp) of FUSE-442 (Fc-EKpp-IL-18AV) with the (a) the MMP2/9 cleavage sequence (GPLGVR) to generate FUSE-486 (Fc-MMP2pp-IL-18AV) and (b) the MMP9/2 cleavage sequence (VHMPLGFLGP) to generate FUSE-487 (Fc-MMP2/9pp-IL-18AV). (Desnoyers et al., Sci Transl Med. 2013 Oct. 16; 5(207):207ra144.) In each case, the MMP to the left of the forward slash preferentially cleaves the aforementioned peptide sequence. For Granzyme B, we substituted the EK cleavage site within the propeptide (pp) of both FUSE-442 (Fc-Ekpp-IL-18AV) and FUSE-424 (Fc-Ekpp-IL-18mut2AV) with a prototypical cleavage site for Granzyme B (IEQD (SEQ ID NO:88)); thus, generating FUSE-485 (Fc-GzmBpp-IL-18AV) and FUSE-462 (Fc-GzmBpp-IL-18mut2AV). FUSE-486, FUSE-487, FUSE-485 and FUSE-462 are illustrated in FIGS. 19A and 19D. Using the HEK-Blue IL-18 reporter cell assay as a readout, we exposed these cells to a titration of (a) FUSE-486 or FUSE-487 (19B) with or without treatment with recombinant human MMP2 or (b) FUSE-485 or FUSE-462 with or without recombinant human Granzyme B (FUSE-485 and FUSE-462) (see FIG. 19D). The biological activity, measured as the EC50-SEAP, of the MMP prodrug fusions, FUSE486 and FUSE487, were greatly attenuated at the orders of up to approximately 5000-fold compared to rhIL8. Following treatment of FUSE-486 with MMP2, the biological activity of the demasked IL-18AV fragment was restored to within 3-fold that of rhIL-18. Next, we added a Granzyme B cleavage site immediately C-terminal to the MMP site in FUSE486 to create FUSE587 (black triangle). As seen in FIG. 19C, FUSE587 was about 3,000-fold attenuated relative to recombinant human IL-18.

Interestingly, we observed that cleavage of FUSE587 with MMP2 released and IL-18AV variant that was still about 100-fold attenuated relative to recombinant IL-18. In contrast, cleavage with Granzyme B released an IL-18AV variant with activity similar activity as recombinant IL-18. Cleavage with Granzyme B results in release of mature IL-18AV without any N-terminal residues constituting an overhang, whereas 11 and 15 amino acid N-terminal polypeptide overhangs remain after cleavage of FUSE486 and FUSE587, respectively, with MMP2. We speculated that these overhangs might be attenuating IL-18AV activity, albeit to a lesser degree than the full size variant propeptide. This phenomenon was further investigated in FIG. 11 and FIG. 13.

FUSE-485 and FUSE-462 were also highly attenuated compared to human recombinant IL-18 at the orders of >15,000-fold (19E and 19F). Treatment with recombinant human Granzyme B (rhGb), designed to cleave off the IL-18AV fragment and the IL-18mut2AV fragment from FUSE-485 and FUSE-462, respectively, led to restoration of biological activity. In both cases, the masked IL-18 variants displayed nearly identical attenuation and the restored biological activity was about 2-fold weaker than recombinant human mature IL-18. Further, we did not observe an appreciable difference between the activity of the demasked IL-18AV and IL-18mut2AV.

Summary tables of the potencies (EC50-SEAP) for each of the test articles is shown below FIGS. 19A-19B and in FIG. 19F.

We next examined whether our finding could form the basis for a plug and play masked IL-18 platform for targeting pro-IL-18 variants to cell surface proteins including but not limited to tumor associated antigens (TAA). As such, we used Cetuximab as our proof-of-concept TAA targeting protein for pro-IL-18 fusion. Cetuximab is an EGFR targeting monoclonal antibody that is commercially used for the treatment of multiple cancer indications including colorectal and head and neck cancer. First, we fused a Granzyme B cleavable pp-IL-18AV onto the C-terminus of Cetuximab's Fc domain. We found that fusion of pp-Gb-IL-18AV onto the CH3 domain of Cetuximab, that forms a natural homodimer, expressed poorly. This was presumably due to the presence of two fabs because a knob into hole IgG1 format in which ppGb-IL-18AV was fused to the C-terminus knob (or hole) and the EGR specific fab was fused to the N-terminal knob (or hole) expressed well. FUSE-517 (Cetuximab_KiH_ppGb-IL-18-AV), as illustrated in panel G consists of the fab from Cetuximab fused to the N-terminus of the Fc-knob and ppGb-IL-18AV fused to the C-terminus of the Fc-knob. Using the HEK-Blue IL-18 reporter cell assay as a readout, we exposed these cells to a titration of FUSE-517 (19H) with or without treatment with rhGb. Relative to rhIL-18, FUSE-517 was highly attenuated at the order of >250,000-fold. Treatment of FUSE-517 with rhGb, designed to cleave off IL-18AV, led to the restoration of biological activity within 2-fold that of rhIL-18.

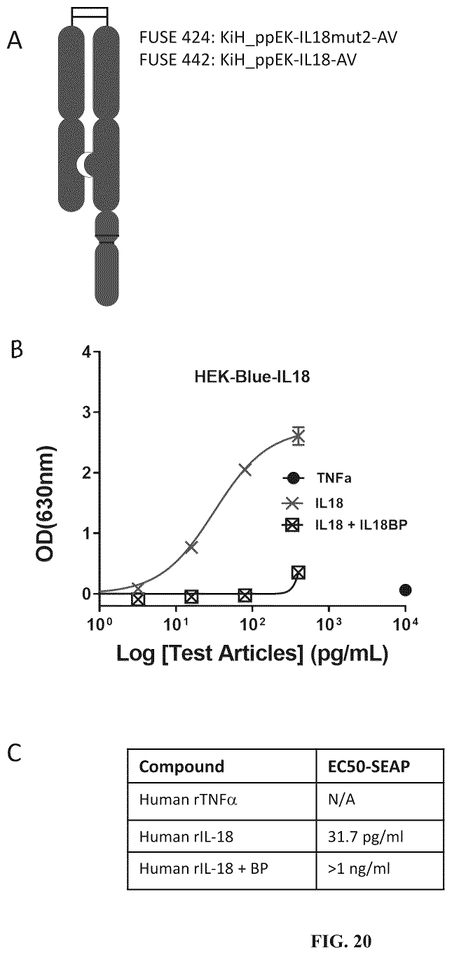

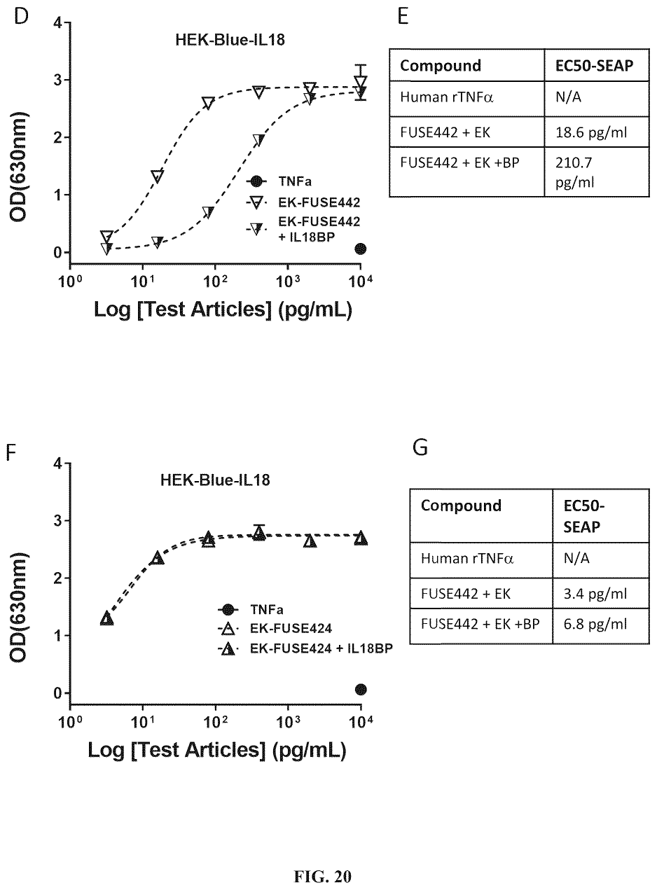

FIG. 19I is summary tables of potencies (EC50-SEAP) related to the data shown in FIG. 9H. Overall, the data indicates strongly that both MMP2 and granzyme B are capable of demasking/activating IL-18AV (and IL-18mut2AV with Granzyme B) in the context of a pp mask fused in-between an IgG CH3 domain and the mature IL-18AV fragment. Given our ability to successfully use other polypeptides that translocate through the ER as masks when fused to the N-terminus of multiple variants of IL-18, our observation suggests strongly that masked fusion proteins of IL-18, its variants or fragments thereof can be engineered to be selectively activated by proteases found in tumors/inflamed tumors including MMPs and Granzymes. As shown in FIG. 20, we examined the susceptibility of select IL-18 variants to attenuation of biological activity by its natural antagonist, IL-18 BP. Panel A of FIG. 20 is an illustration of the IL-18 fusion proteins examined. That is. FUSE-442 (Fc-Ekpp-IL-18AV) and FUSE-424 (Fc-Ekpp-IL-18mut2AV). Both variants contain the same cysteine to valine substitutions, however, FUSE-424 harbors a mature IL-18AV (termed IL-18mut2AV) downstream of the polypeptide (pp) that includes the following mutations: M87K, K89G, M696L and M149V. These four residues within the mature 18 kDa fragment of IL-18 were previously described as important to binding/masking of wild type IL-18. We therefore hypothesized that substitution of these residues would reduce binding of IL-18 BP such that IL-18Avmut2 would maintain biological activity in the presence of IL-18BP, which is often over-represented relative to IL-18 in the tumor microenviromnent (TME). As in previous examples, HEK Blue IL-18 was used to assess biological activity via MYD88 driven SEAP and potency reported as the EC50-SEAP. FUSE-442 (Fc-Ekpp-IL-18AV) and FUSE-424 (Fc-Ekpp-IL-18mut2AV) were treated with EK to release mature IL-18AV or IL-18mut2AV, respectively. Each cleavage product was then titrated from 1 μg/ml to 1 μg/ml in media alone or media containing 1.25 μg/ml human recombinant IL-18-BP (hrIL-18-BP). Human recombinant IL-18 (hrIL-18) was used as the reference molecule for wild type inhibition of HEK Blue IL-18 reporter activity mediated by rhIL-18BP. Panels B (rhIL-18). D (FUSE-442), and F (FUSE-424) are non-linear x-y plots of SEAP release (IL-18R reporter activity) on the y-axis versus the concentration of test article on the x-axis in the presence or absence of IL-18BP. A summary of biological potencies (EC50-SEAP) relevant to each graph is shown as panels C, E, and G (beneath each x-y plot). As is well documented, we observed strong attenuation of rhIL-18 by rhIL-18BP in the order of at least 300-1000-fold. For IL-18AV released from FUSE442, we observed about 100 to 150-fold attenuation of biological activity (i.e., ~3-6 fold less than rhIL-18) suggesting that apparent affinity of IL-18AV for IL-18BP is weaker than that of rhIL-18 for IL-18BP. Importantly, the cleavage product of FUSE424. IL-18mut2AV, appeared resistant to biological attenuation by rhIL-18BP. As seen in panel F, we observed no appreciable difference in biological activity between IL-18mut2AV released from FUSE424 and the same molecule exposed to rhIL-18BP. This data suggests strongly that IL-18mut2AV binds with far weaker apparent affinity to rhIL-18BP compared to IL-18AV or rhIL-18. As such, a masked version of IL-18mut2AV designed to be released by proteases present in the TME including but not limited to MMP2, 9 and 14 and/or proteases released in inflamed tumors including but not limited to Granzymes A, B and M may be predicted to function in the presence of IL-18BP to promote anti-tumor activity via multiple pathways including but not limited to IFNγ mediated Th1 and Tc1 activity.

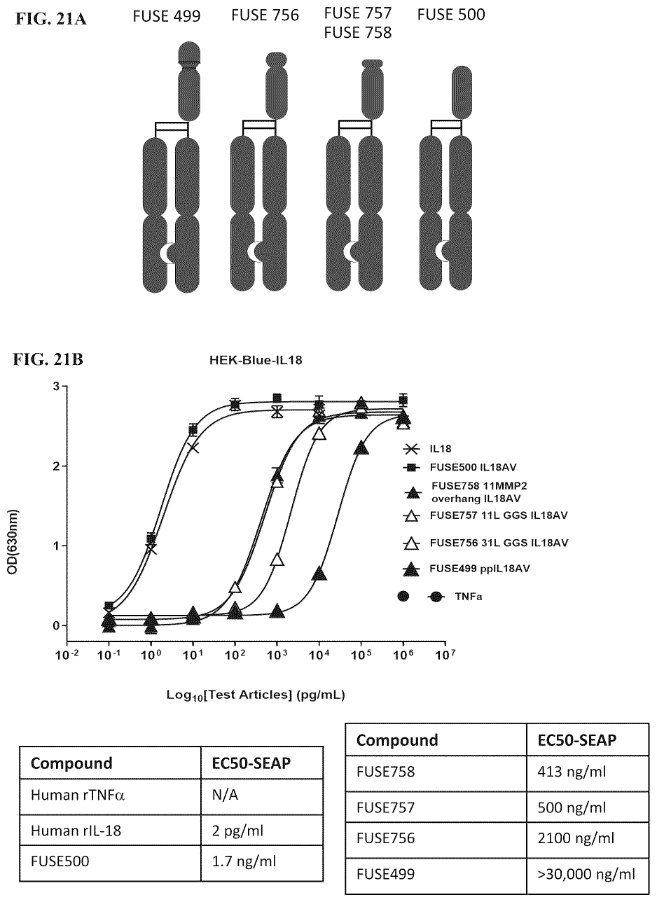

As shown in FIG. 21, we examined the impact of the size of polypeptides fused to the N-terminus of mature IL-18 on the biological activity of a single IL-18AV fused to the N-terminus of IgG1 Fc. Panels A are exemplary illustrations of IL-18AV fused on the knob of a knob into hole IgG1-Fc domain with different size polypeptides ranging from a propeptide variant (FUSE-499; described in FIG. 18) to 35 amino acids (FUSE756) to 15 amino acids (FUSE757 and FUSE758). To allow for translocation of all constructs into the Endoplasmic Reticulum (ER) of mammalian cells and hence expression/secretion, the signal peptide from the Ig Kappa chain (IgK leader) was encoded upstream of the polypeptides (and cleaved of the signal peptidase in the ER). The polypeptides of FUSE756 and FUSE757 are made up of a series of glycine and serine residues whereas that of FUSE758 is the sequence of the overhang generated by MMP2 mediated cleavage of the MMP cleavage site. KPLGLQARVVGGGG. In these three cases, the C-terminus of the N-terminal polypeptides also incorporated the Granzyme B site, IEQD. Using the HEK-Blue IL-18 reporter cell assay as a readout, we examined the capacity of the different size polypeptides to attenuate/mask IL-18AV-Fc (FUSE500; closed squares) or rhIL-18 (black cross hatches “X”). All polypeptides fused to the N-terminus of mature IL-18AV reduced biological activity, measured as EC50-SEAP, by at least 100-fold. FUSE499 (closed triangles) was the most attenuated (>10,000-fold). FUSE757 (closed diamonds) and 758 (open reverse triangle), both of which included 11 amino acid polypeptides fused to the N-terminus of mature IL-18AV, were about 250-fold less biologically active relative to FUSE500 or rhIL-18. FUSE756 (open triangles), which contained a polypeptide of 31 amino acids polypeptides fused to the N-terminus of mature IL-18AV was approximately 1000-fold less biologically active relative to FUSE500 or rhIL-18. Thus, all polypeptides tested and larger proteins including HSA and fragments of IgG from previous examples, fused to the N-terminus of mature IL-18AV, regardless of size or amino acid constitution, attenuated the biological activity of mature IL-18. The degree of attenuation appears to correlate positively with the size of the polypeptide/protein fused to the N-terminus of IL-18.

### Example 21

### Techniques and Procedures

HEK-Blue-IL-18 Cell Activation Assay with IL-18 or FUSE Proteins

HEK-BLUE™-IL-18 cells from Invivogen were maintained in culture medium (DMEM medium with 4.5 g/L glucose. 2 mM L-Glutamine. 10% (v/v) heat-inactivated fetal bovine serum, 100 U/mL penicillin, 100 μg/mL streptomycin, 100 μg/mL Normocin and 1×HEK-blue selection reagent). HEK-BLUE™ IL-18 cells are engineered from the human embryonic kidney 293 (HEK293) cell line to stably express genes encoding the IL-18 receptor (IL-18R) and IL-18 receptor accessory protein (IL-18RAP) and express an NF-Kb/AP-1-inducible secreted embryonic alkaline phosphatase (SEAP) reporter gene, therefore being useful for detection of bioactive IL-18 by monitoring the activation of the NF-Kb and AP-1 pathways via quantification in the supernatant of the SEAP level (which is produced upon activation of NF-Kb) with a solution such as QUANTI-BLUE™ Solution. In addition, the responses to human TNF-α and IL-1β have been blocked in HEK-BLUE™ IL-18 cells, and so the HEK-BLUE™ IL-18 cells are responsive specifically to IL-18.

On the day of experiment setup, HEK-BLUE-IL-18 cells were gently rinsed twice with pre-warmed phosphate buffered saline (PBS) and then detached in PBS by tapping the flask. Detached HEK-Blue-IL-18 cells were resuspended in pre-wanned testing medium (DMEM with 4.5 g/L glucose, 2 mM L-Glutamine, 10% (v/v) heat-inactivated FBS, 100 U/mL penicillin, and 100 μg/mL streptomycin) at the density of 5×10⁵ cell per mL. To seed the cells (50000 cells per well), 100 μL of resuspended HEK-Blue-IL-18 cells were added to designated wells in 96-well plate.

For protein preparation, FUSE proteins were diluted hi testing medium from 500000 μg/mL to 6.4 pg/mL (2× of final concentration) by 5-fold serial dilution. IL-18 was also diluted in testing medium from 800 μg/mL to 1.28 pg/mL (2× of final concentration) by 5-fold serial dilution. To treat HEK-Blue-IL-18 cells, 100 μL of prepared IL-18 or FUSE proteins were added to the designated wells with 50000 cells in the 96-well plate, and then the proteins were gently mixed the cells. The 96-well plate was then incubated at 37° C. with 5% CO₂ for 24 hours.

After 24-hour activation, HEK-Blue-IL-18 cells released secreted alkaline phosphatase in the supernatant. 20 μL of HEK-Blue-IL-18 cell culture supernatant was transferred to a new 96-well plate. Meanwhile, QUANTI-Blue solution from Invivogen was prepared by adding 1 mL of QB reagent and 1 mL of QB buffer to 98 mL of sterile water. 180 μL of prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the OD at 630 nm using a spectrophotometer.

HEK-Blue-IL-18 Cell Activation Assay with Enterokinase (EK)-Cleaved FUSE Proteins

HEK-Blue-IL-18 cells from Invivogen were maintained in culture medium. On the day of experiment setup, HEK-Blue-IL-18 cells were gently rinsed twice with pre-warmed phosphate buffered saline (PBS) and then detached in PBS by tapping the flask. Detached HEK-Blue-IL-18 cells were resuspended in pre-warmed testing medium at the density of 5×10⁶ cell per mL. To seed the cells (50000 cells per well), 100 μL of resuspended HEK-Blue-IL-18 cells were added to designated wells in 96-well plate.

For FUSE protein cleavage. 4 μg of FUSE protein was mixed with 80 ng of EK enzyme and 2 μL of 10×PBS in a tube. Sterile water was added to the tube to bring the total reaction volume to 20 μL. The tube was then incubated at 25° C. for 40 minutes. After 40-minute incubation, cleaved FUSE proteins were diluted in testing medium from 500000 μg/mL to 6.4 pg/mL (2× of final concentration) by 5-fold serial dilution. IL-18 was also diluted in testing medium from 800 μg/mL to 1.28 pg/mL (2× of final concentration) by 5-fold serial dilution. To treat HEK-Blue-IL-18 cells, 100 μL of prepared IL-18 or cleaved FUSE proteins were added to the designated wells with 50000 cells in the 96-well plate and then gently mixed the cells with proteins. The 96-well plate was then incubated at 37° C. with 5% CO2 for 24 hours.

After 24-hour activation, 20 μL of HEK-Blue-IL-18 cell culture supernatant with secreted alkaline phosphatase was transferred to a new 96-well plate. Meanwhile, QUANTI-Blue solution was prepared by adding 1 mL of QB reagent and 1 mL of QB buffer to 98 mL of sterile water. 180 μL of prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the OD at 630 nm using a spectrophotometer.

HEK-Blue-IL-18 Cell Activation Assay with Matrix Metalloproteinase (MMP)-Cleaved FUSE Proteins

HEK-Blue-IL-18 cells from Invivogen were maintained in culture medium. On the day of experiment setup, HEK-Blue-IL-18 cells were gently rinsed twice with pre-warmed phosphate buffered saline (PBS) and then detached in PBS by tapping the flask. Detached HEK-Blue-IL-18 cells were resuspended in pre-warmed testing medium at the density of 5×10⁶ cell per mL. To seed the cells (50000 cells per well), 100 μL of resuspended HEK-Blue-IL-18 cells were added to designated wells in 96-well plate.

For FUSE protein cleavage, I pg of FUSE protein was mixed with 280 ng of MMP2 or MMP9 enzyme and 2.8 μL of 10× assay buffer (500 mM Tris, 100 mM CaCl₂), 1500 mM NaCl, 0.5% (w/v) Brij-35, pH 7.5),) in a tube. Sterile water was added to the tube to bring the total reaction volume to 28 μL. The tube was then incubated at 37° C. for 2 hours. After 2-hour incubation, cleaved FUSE proteins were diluted in testing medium from 500000 μg/mL to 6.4 pg/mL (2× of final concentration) by 5-fold serial dilution. IL-18 was also diluted in testing medium from 800 μg/mL to 1.28 pg/mL (2× of final concentration) by 5-fold serial dilution. To treat HEK-Blue-IL-18 cells, 100 μL of prepared IL-18 or cleaved FUSE proteins were added to the designated wells with 50000 cells in the 96-well plate and then gently mixed the cells with proteins. The 96-well plate was then incubated at 37° C. with 5% CO2 for 24 hours.

After 24-hour activation, 20 μL of HEK-Blue-IL-18 cell culture supernatant with secreted alkaline phosphatase was transferred to a new 96-well plate. Meanwhile, QUANTI-Blue solution was prepared by adding 1 mL of QB reagent and 1 mL of QB buffer to 98 mL of sterile water. 180 μL of prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the OD at 630 nm using a spectrophotometer.

HEK-Blue-IL-18 Cell Activation Assay with Caspase I-Cleaved FUSE Proteins

HEK-Blue-IL-18 cells from Invivogen were maintained in culture medium. On the day of experiment setup, HEK-Blue-IL-18 cells were gently rinsed twice with pre-warmed phosphate buffered saline (PBS) and then detached in PBS by tapping the flask. Detached HEK-Blue-IL-18 cells were resuspended in pre-warmed testing medium at the density of 5×10⁵ cell per mL. To seed the cells (50000 cells per well), 100 μL of resuspended HEK-Blue-IL-18 cells were added to designated wells in 96-well plate.

For FUSE protein cleavage, 14 μg of FUSE protein was mixed with 0.5 unit of Caspase I enzyme and 2.8 μL of 10× assay buffer (500 mM Hepes, pH 7.2, 500 mM NaCl. 1% Chaps, 100 mM EDTA, 50% Glycerol, and 100 mM DTT) in a tube. Sterile water was added to the tube to bring the total reaction volume to 28 μL. The tube was then incubated at 37° C. for 2 hours. After 2-hour incubation, cleaved FUSE proteins were diluted in testing medium from 500000 μg/mL to 6.4 pg/mL (2× of final concentration) by 5-fold serial dilution. IL-18 was also diluted in testing medium from 800 μg/mL to 1.28 pg/mL (2× of final concentration) by 5-fold serial dilution. To treat HEK-Blue-IL-18 cells, 100 μL of prepared IL-18 or cleaved FUSE proteins were added to the designated wells with 50000 cells in the 96-well plate and then gently mixed the cells with proteins. The 96-well plate was then incubated at 37° C. with 5% CO₂ for 24 hours.

After 24-hour activation, 20 μL of HEK-Blue-IL-18 cell culture supernatant with secreted alkaline phosphatase was transferred to a new 96-well plate. Meanwhile, QUANTI-Blue solution was prepared by adding 1 mL of QB reagent and 1 mL of QB buffer to 98 mL of sterile water. 180 μL of prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the OD at 630 nm using a spectrophotometer.

HEK-Blue-IL-18 Cell Activation Assay with Granzyme B-Cleaved FUSE Proteins

HEK-Blue-IL-18 cells from Invivogen were maintained in culture medium. On the day of experiment setup, HEK-Blue-IL-18 cells were gently rinsed twice with pre-warmed phosphate buffered saline (PBS) and then detached in PBS by tapping the flask. Detached HEK-Blue-IL-18 cells were resuspended in pre-wanned testing medium at the density of 5×10⁵ cell per mL. To seed the cells (50000 cells per well), 100 μL of resuspended HEK-Blue-IL-18 cells were added to designated wells in 96-well plate.

Mature active human granzyme B was generated by cleaving human pro-granzyme B using EK enzyme. In brief, 4 μg of human pro-granzyme B was mixed with 40 ng of EK enzyme and 2 μL of 10×PBS in a tube. Sterile water was added to the tube to bring the total reaction volume to 20 μL. The tube was then incubated at 25° C. for 40 minutes. After 40-minute incubation, activated human granzyme B was used to cleave FUSE proteins. In brief, 10 g of fuse proteins were mixed with 1 μg of activated human granzyme B in assay buffer (50 mM HEPES (pH 7.4), 100 mM NaCl, 0.1% CHAPS, 1 mM EDTA, 10% Glycerol) and incubated for certain time indicated in each experiment at 37° C.

Granzyme B-cleaved fuse proteins were then diluted in testing medium from 500000 μg/mL to 6.4 pg/mL (2× of final concentration) by 5-fold serial dilution. IL-18 was also diluted in testing medium from 800 μg/mL to 1.28 pg/mL (2× of final concentration) by 5-fold serial dilution. To treat HEK-Blue-IL-18 cells, 100 μL of prepared IL-18 or cleaved FUSE proteins were added to the designated wells with 50000 cells in the 96-well plate and then gently mixed the cells with proteins. The 96-well plate was then incubated at 37° C. with 5% CO₂ for 24 hours.

Non-cleaved or cleaved FUSE proteins were prepared in testing medium from 1000000 μg/ml to 12.8 pg/ml (4× of final concentration) by 5-fold serial dilution. IL-18BP was diluted in testing medium at the concentration of 5,000,000 μg/ml (4× of final concentration) by. To treat HEK-Blue-IL-18 cells, 50 μL of prepared non-cleaved or cleaved FUSE proteins and 50 μL of prepared IL-18BP were mixed, incubated for 1 hour, and then added to the designated wells with 50000 cells in the 96-well plate and then gently mixed the cells with proteins. The 96-well plate was then incubated at 37° C. with 5% CO2 for 24 hours. Since our fusion proteins have attenuated binding to the IL-18R complex, we conceive that they will also have reduced binding to IL-18BP, compared to cleaved FUSE proteins, because both IL-18R and IL-18BP compete for binding to IL-18. The recent crystal structures of binary and ternary complexes of hIL-18 with its receptors have shown that IL-18BP competes directly with the hIL-18Rα D3 domain for binding hIL-18, overlapping the previously identified hIL-18 binding site II (Krumm, et al., Acta Crystallogr F Struct Biol Commun. 2015 Jun. 1: 71(Pt 6): 710-717).

After 24-hour activation, 20 μL of HEK-Blue-IL-18 cell culture supernatant with secreted alkaline phosphatase was transferred to a new 96-well plate. Meanwhile, QUANTI-Blue solution was prepared by adding 1 mL of QB reagent and 1 mL of QB buffer to 98 mL of sterile water. 180 μL of prepared QUANTI-Blue solution was then added to the 96-well plate with 20 μL of HEK-Blue-IL-18 cell culture supernatant and incubated at 37° C. for 4 hours. The level of secreted alkaline phosphatase related to HEK-Blue-IL-18 cell activation was detected by measuring the OD at 630 nm using a spectrophotometer.

### Binding Assay by Biolayer Interferometry

Coating proteins were prepared in 1×PBS with 0.02% Tween-20 with the final concentration of 15 μg/ml. Capturing protein were also prepared, and serial diluted (4-fold dilution ranging from 400 nM to 1.6 nM) in 1×PBS with 0.02% Tween-20. The biosensors were pre-moistened in 200 μL of 1×PBS with 0.02% Tween-20 for 10 minutes. Meanwhile, Octet BLI system (ForteBio) was prewarmed for 30 minutes and the flow rate was set to 1000 rpm. The biosensors (Capture biosensor) were soaked in 250 μL of 1×PBS with 0.02% Tween-20 for 60 seconds at 30° C. to get an initial baseline reading. After the 60-second baseline reading, the biosensors were exposed to coating proteins for 300 seconds at 30° C. for the association between antibody and the biosensors (coupling coating proteins with biosensor). The biosensors with coating proteins were then exposed to capturing proteins in 250 μL of 1×PBS with 0.02% Tween-20 at 30° C. for 300 seconds for the association reaction between the coating proteins and capturing proteins (association curve). After 300-second association reaction between coating proteins and capturing proteins, the biosensors with coating proteins and capturing proteins were then exposed to 250 μL of 1×PBS with 0.02% Tween-20 at 30° C. for 300 seconds for the dissociation reaction between coating proteins and capturing proteins (dissociation curve). For binding to IL18BP, each IL18 mutant was coated onto AHC biosensor tips and probed with recombinant His tagged IL-18BP at concentrations ranging from 400 nM to 1.6 nM. For binding to IL18Rα, His tagged IL18Ra was coated into nickel biosensor tips and probed with recombinant each IL18 mutant at concentrations ranging from 400 nM to 1.6 nM. The binding affinity was calculated by the built-in data fitting algorithm.

### Example 22

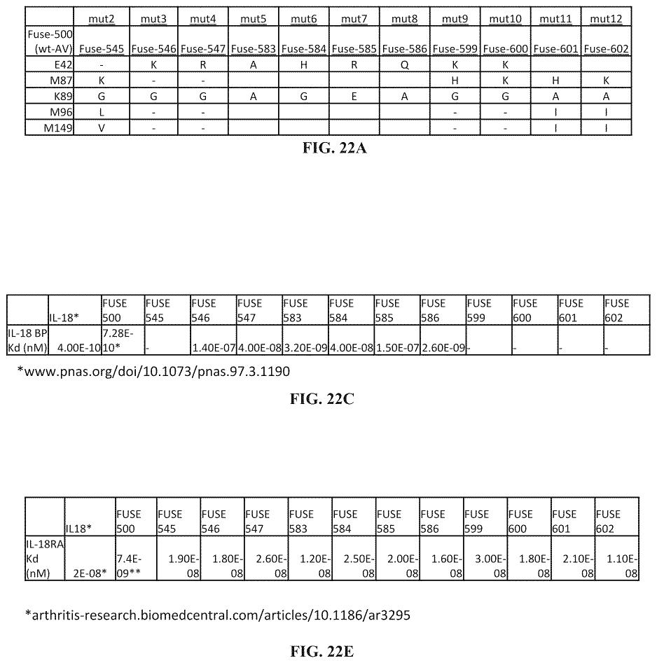

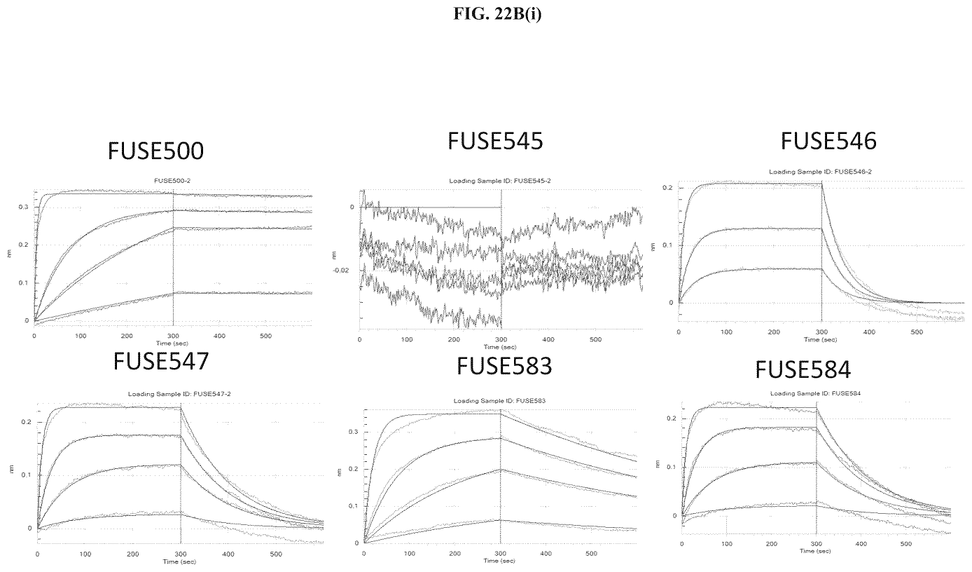

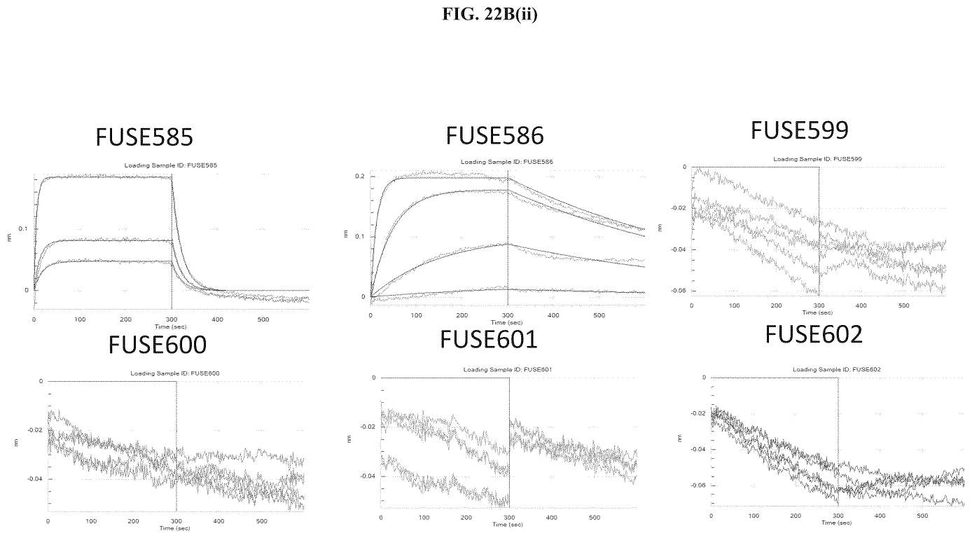

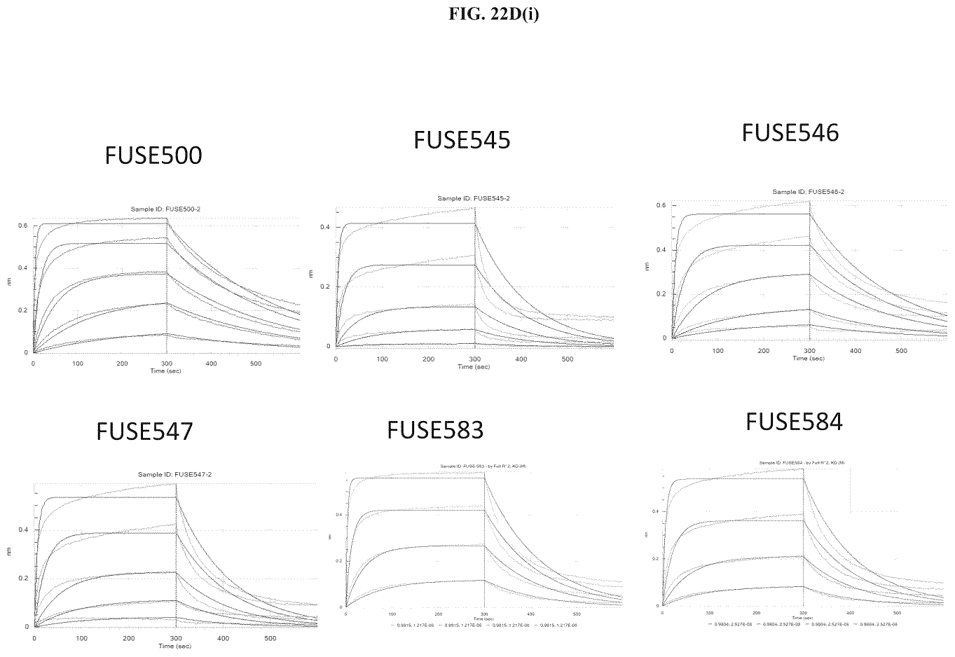

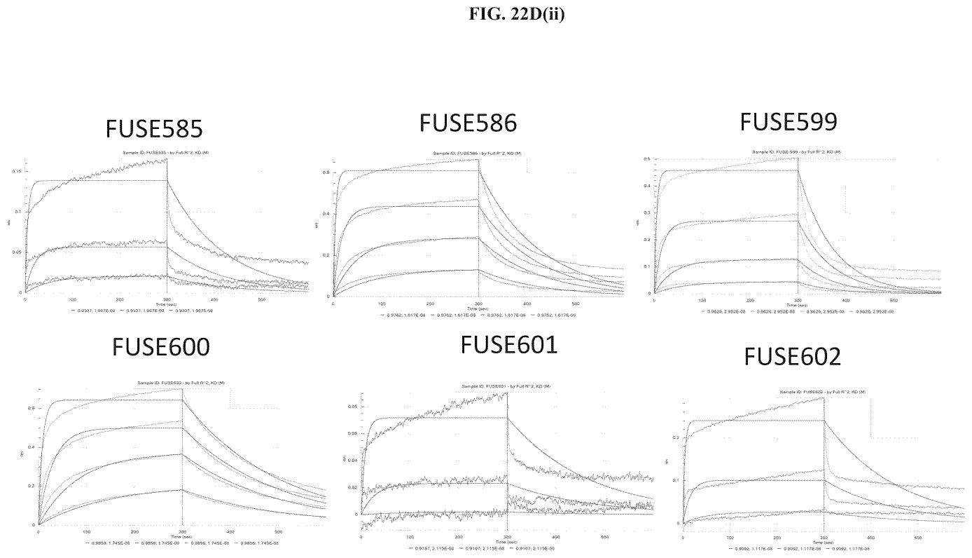

As shown in FIG. 22, we engineered eleven mutants of human IL-18 (IL-18AV shown) and measured their ability to bind recombinant human IL-18BP and recombinant human IL-18RA (also termed IL-18Rα). All test articles were generated as Fc fusion proteins with the IL-18 variant fused to the N-terminus of the Fc (see FIG. 11A). The locations and amino acid substations associated with each variant are depicted in FIG. 22A.

To assess binding to human IL18BP or human IL18Rα, kinetic binding graphs were generated via Bio-Layer Interferometry (BLI) using an Octet system (ForteBio). For binding to IL18BP, each IL18 mutant was coated onto AHC biosensor tips and probed with recombinant His tagged IL-18BP at concentrations ranging from 400 nM to 1.6 nM (FIG. 22B). For binding to IL18Rα, His tagged IL18Ra was coated into nickel biosensor tips and probed with recombinant each IL18 mutant at concentrations ranging from 400 nM to 1.6 nM (FIG. 22D). Summary tables for binding to IL-18BP and IL18-RA (binding affinity (KD), on rate (k-on) and off rate (k-dis)) are shown in FIGS. 22C and 22E, respectively.

All eleven IL-18 mutants bound to IL18BP with weaker affinity than FUSE500 (wild type human IL-18-AV-Fc). Five mutants were associated with no appreciable binding. These are FUSE545, FUSE599, FUSE600, FUSE601 and FUSE602.

In contrast, all eleven mutants maintained appreciable binding to IL18RA. The affinities ranged from 11 nM to 30 nM, which was only between 1.5-fold and 4-fold weaker than the affinity of FUSE500 for IL-18RA (7.4 nM).

### Example 23

As shown in the table below, we tabulated the binding affinities of eleven mutant variants of human IL-18 (IL-18AV shown) to human IL18BP and human IL18Rα. Each IL-18 protein was generated as an Fc fusion proteins whereby the IL-18 variant was fused to the N-terminus of the Fc (see FIG. 21A).

| | | IL-BP Kd | IL-18Ra Kd |
|---|---|---|---|
| Protein | Mutant | (nM) | (nM) |
| IL18 | | 4E−10*** | 2E−08**** |
| FUSE 500 | Wild type AV | 7.28E−10* | 7.4E−09** |
| FUSE 545 | mut2 | — | 1.9E−08 |
| FUSE 546 | mut3 | 1.4E−07 | 1.8E−08 |
| FUSE 547 | mut4 | 4.0E−08 | 2.6E−08 |
| FUSE 583 | mut5 | 3.2E−09 | 1.2E−08 |
| FUSE 584 | mut6 | 4.0E−08 | 2.5E−08 |
| FUSE 585 | mut7 | 1.5E−07 | 2.0E−08 |
| FUSE 586 | mut8 | 2.6E−09 | 1.6E−08 |
| FUSE 599 | mut9 | — | 3.0E−08 |
| FUSE 600 | mut10 | — | 1.8E−08 |
| FUSE 601 | mut11 | — | 2.1E−08 |
| FUSE 602 | mut12 | — | 1.1E−08 |

*mean of 11 measurements,
**mean of 9 measurements
***www.pnas.org/doi/10.1073/pnas.97.3.1190
****arthritis-research.biomedcentral.com/articles/10.1186/ar3295

### Example 24

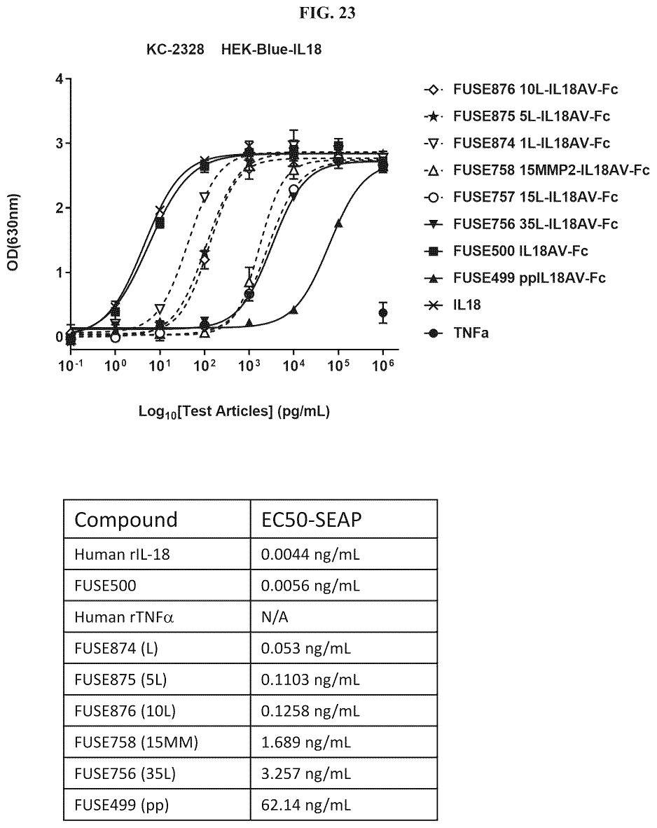

As shown in FIG. 23, we examined the impact of the size of polypeptides fused to the N-terminus of mature IL-18 on the biological activity of a single IL-18AV fused to the N-terminus of IgG1 Fc.

A single N-terminal amino acid (FUSE874: downward open triangles) and different size polypeptides ranging from five amino acids (FUSE875) to the propeptide variant of FUSE-499 (upward closed triangles; see FIG. 21 for description) were investigated. FUSE500 was used as the fully active control without a N-terminal polypeptide.

To allow for translocation of all constructs into the Endoplasmic Reticulum (ER) of mammalian cells and hence expression/secretion, the signal peptide from the Ig Kappa chain (IgK leader) was encoded upstream of the polypeptides (and cleaved off the signal peptidase in the ER).

The polypeptides of FUSE875 (5 residues; closed stars), FUSE876 (10 residues; open diamonds), FUSE757 (15 residues; open circles), FUSE756 (35 residues; downward closed triangles) are made up of a series of glycine and serine residues. The N-terminal polypeptide associated with FUSE758 (open upward triangles) is the sequence of the overhang generated by MMP2 cleavage of FUSE486 (see FIG. 19). For FUSE756, FUSE757 and FUSE758, the final four amino acids of the N-terminal peptide consisted of the Granzyme B cleavage site, IEQD (SEQ ID NO:88) (see FIG. 21).

Using the HEK-Blue IL-18 reporter cell assay as a readout, we examined the capacity of the different size polypeptides to attenuate/mask IL-18AV-Fc (FUSE500; closed squares) or rhIL-18 (black crosses “X”). All polypeptides and the single amino acid (serine) fused to the N-terminus of mature IL-18AV reduced biological activity, measured as EC50-SEAP, by at least 10-fold.

The degree of attenuation increased as the size of the polypeptide increased, such that the rank order of attenuation was observed as FUSE499>FUSE756, FUSE757, FUSE758>FUSE876, FUSE875>FUSE874. A summary table of potencies (EC50-SEAP) is shown beneath the non-linear x-y graph.

### Example 24

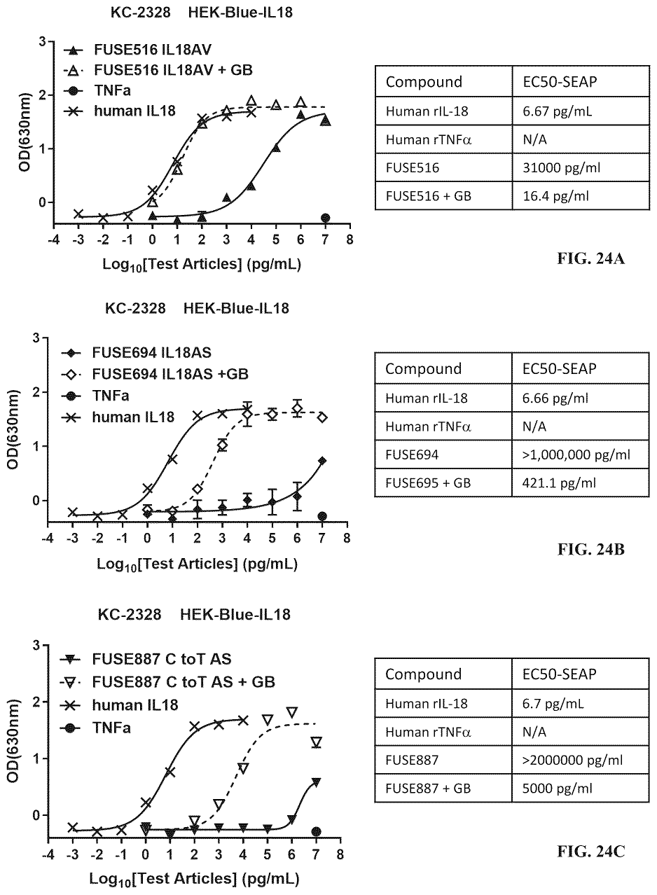

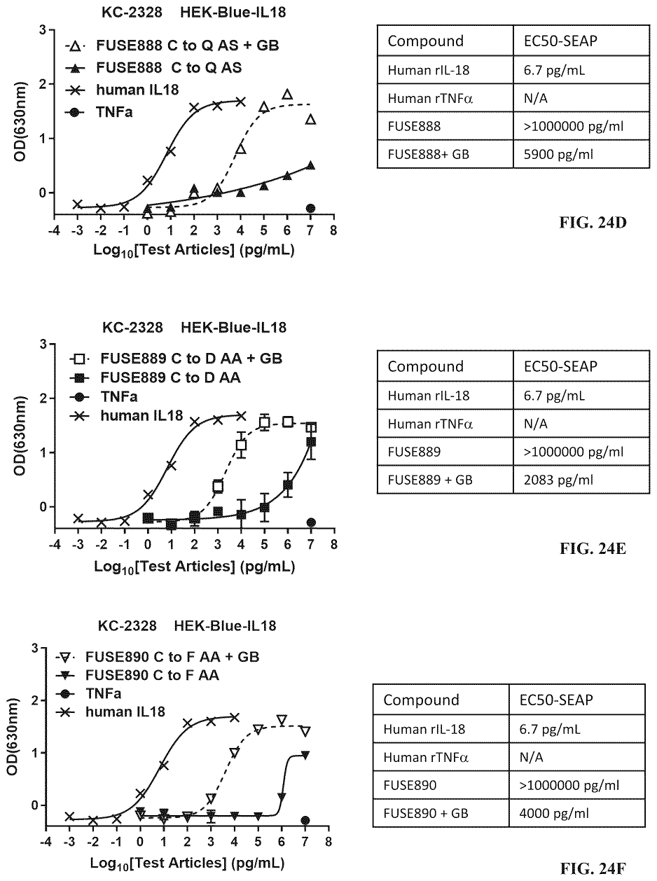

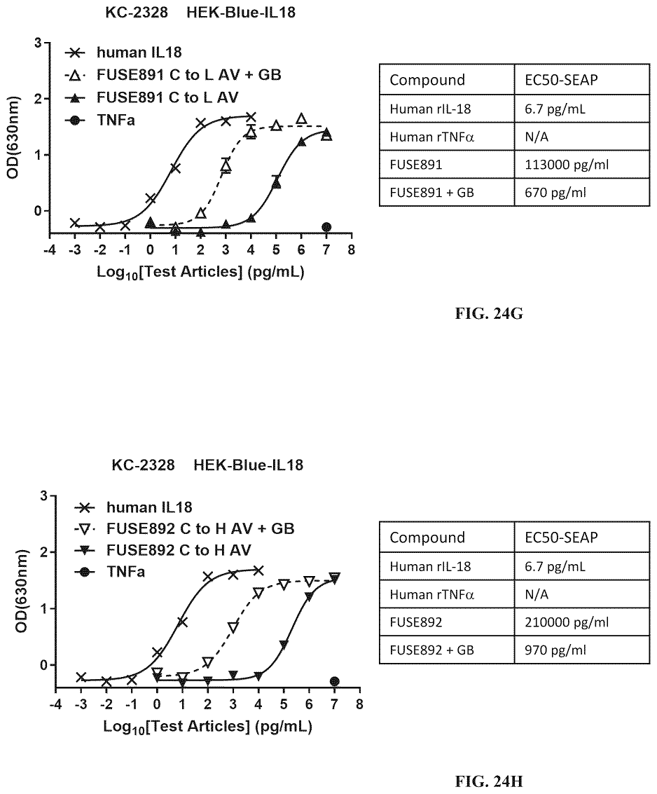

As shown in FIG. 24A-24H, we examined the impact of the substituting the cysteine residue in the pro-peptide and cysteine residues in the mature IL18, which were fused together to form the pro-IL-18 variant cassette, on the biological activity of each variant using the HEK Blue IL18 assay system. Unless otherwise stated, the proteins were generated such that the propeptide-IL-18 variant was fused to the C-terminal knob or hole of a knobs into holes human IgG1 Fc as previously depicted in FIG. 1A with a Granzyme B cleavage site added between the pro-peptide variant and the mature IL18 variant. The three variants assessed in FIG. 1 contained serine substitutions (FUSE480), alanine substitutions (FUSE481) or valine substitutions (FUSE442) at all the cysteine residues in pro-IL-18. Mature IL-18 variants released from FUSE442 and FUSE481 were about as active as recombinant human IL-18 whereas mature IL18 released from FUSE480 (serine substituted) was approximately 100-fold attenuated versus recombinant human IL-18 (see FIGS. 1B-1D).

For this example, the variant of pro-IL-18 in which all the cysteines were substituted for valine included an N-terminus EGFR specific VHH (FUSE516; closed triangle) and the variant of pro-IL-18 in which all the cysteines were substituted for serine included an N-terminus PD-1 specific Fab (FUSE694; closed diamond). As in FIG. 1, biological activity of the pro-IL18 test articles were assessed using HEK Blue IL18. Each was tested as an intact untreated protein or following exposure to recombinant human Granzyme B.

FIG. 24A shows the results with FUSE516 (closed triangle), in which the cysteine residue in its pro-peptide variant is replaced with valine and all the cysteines in its mature IL18 variant are replaced by valine. Intact FUSE516 is about 1000-fold attenuated relative to recombinant human IL18 and that the mature IL18 variant released from FUSE516 (open triangle) by Granzyme B is approximately as active as recombinant human IL18.

FIG. 24B shows the results with FUSE694 (closed diamond), in which the cysteine residue in its pro-peptide variant is replaced with serine and all of the cysteines in its mature IL18 variant are replaced by serines. Intact FUSE694 is greater than 10,000-fold attenuated relative to recombinant human IL18 and that the mature IL18 variant released from FUSE694 (open diamond) by Granzyme B is approximately 60-fold less active than recombinant human IL18.

FIG. 24C shows the results with FUSE887 (closed reverse triangle), in which the cysteine residue in its pro-peptide variant is replaced with threonine and all the cysteines in its mature IL18 variant are replaced by serines. Intact FUSE887 is greater than 10,000-fold attenuated relative to recombinant human IL18 and that the mature IL18 variant released from FUSE887 by Granzyme B (open reverse triangle) is approximately 100-fold less active than recombinant human IL18.

FIG. 24D shows the results with FUSE888 (closed triangle), in which the cysteine residue in its pro-peptide variant is replaced with glutamine and all the cysteines in its mature IL18 variant are replaced by serines. Intact FUSE888 is greater than 10,000-fold attenuated relative to recombinant human IL18 and that the mature IL18 variant released from FUSE888 (open triangle) by Granzyme B is approximately 100-fold less active than recombinant human IL18.

FIG. 24E shows the results with FUSE889 (closed square), in which the cysteine residue in its pro-peptide variant is replaced with aspartic acid and all the cysteines in its mature IL18 variant are replaced by alanines. Intact FUSE889 is greater about 10,000-fold attenuated relative to recombinant human IL18 and that the mature IL18 variant released from FUSE889 by Granzyme B (open square) is approximately 100-fold less active than recombinant human IL18.

FIG. 24F shows the results with FUSE890 (closed reverse triangle), in which the cysteine residue in its pro-peptide variant is replaced with phenylalanine and all of the cysteines in its mature IL18 variant are replaced by alanines. Intact FUSE890 is about than 10,000-fold attenuated relative to recombinant human IL18 and that the mature IL18 variant released from FUSE890 by Granzyme B (open reverse triangle) is approximately 100-fold less active than recombinant human IL18.

FIG. 24G shows the results with FUSE891 (closed triangle), in which the cysteine residue in its pro-peptide variant is replaced with isoleucine and all the cysteines in its mature IL18 variant are replaced by valines. Intact FUSE891 is about 3,000-fold attenuated relative to recombinant human IL18 and that the mature IL18 variant released from FUSE891 (open triangle) by Granzyme B is approximately 100-fold less active than recombinant human IL18.

FIG. 24H shows the results with FUSE892 (closed reverse triangle), in which the cysteine residue in its pro-peptide variant is replaced with histidine and all the cysteines in its mature IL18 variant are replaced by valines. Intact FUSE892 is greater about 3,000-fold attenuated relative to recombinant human IL18 and that the mature IL18 variant released from FUSE892 (open reverse triangle) by Granzyme B is approximately 100-fold less active than recombinant human IL18.

Summary tables of potency (EC50-SEAP) are show to the right of each non-linear x-y plot.

### Example 25

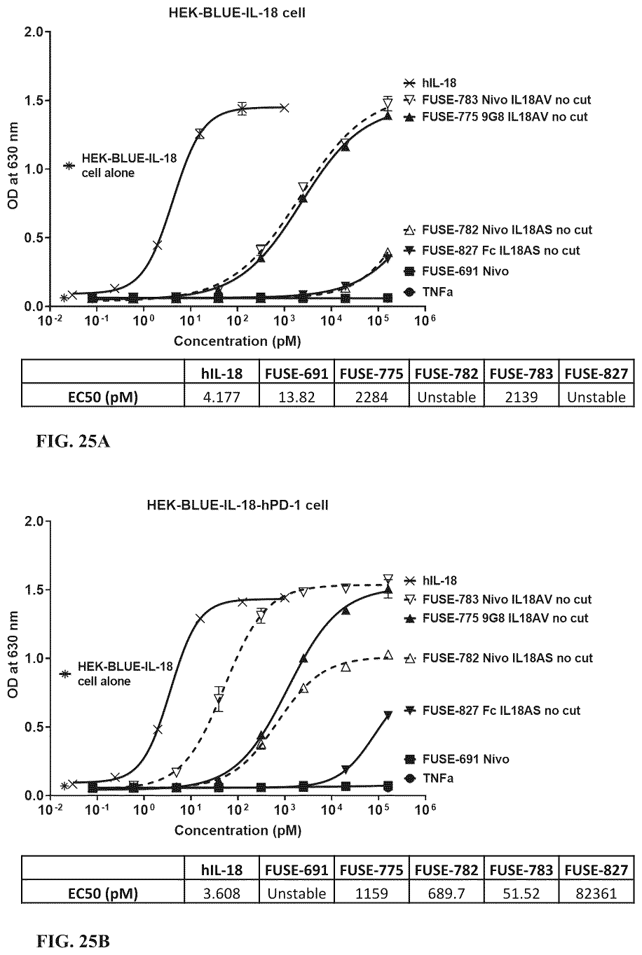

As shown in FIG. 25A-25B, we examined the impact of targeting pro-IL18 to within close proximity of its receptor complex (i.e., “cis activity”). Pro-IL-18 variants tested were (a) that in which all the cysteine residues were substituted with serines (pro-IL18AS, FUSE782 and FUSE827) and (b) that in which all the cysteines were substituted with valines (pro-IL18AV, FUSE783 and FUSE785). Pro-IL18AS or pro-IL18AV were fused to the N-terminal knob or hole of a PD1 specific knobs into holes antibody (FUSE782 and FUSE783). The fab for these fusion proteins was derived from nivolumab. To test whether targeting the pro-IL18 variants to PD1 decorated on the same cell that expressed the IL-18R complex (cis effect) enhanced biological activity compared to cells that were not decorated with PD-1 (trans effect), (1) we used HEK Blue IL18 as our IL18R complex positive reporter system, and (2) either used the cell without modification or decorated the cell line with the ectodomain of PD-1 using a bispecific antibody that contains a CD46 specific fab (clone YS5) on the N-terminal hole and the PD-1 ectodomain on the N-terminal knob (FUSE986). CD46 was chosen because of its reported expression the parental HEK 293 cell line (jitc.bmj.comn/content/6/1/55), which we confirmed on HEK Blue IL18 (not shown). We also generated pro-IL18AS (FUSE827) and pro-IL18AV (FUSE775) that were functional non targeted in this and could only function in trans. FUSE827 contained no targeting domain (i.e., Fc only) and FUSE775 replaced the PD-1 specific fab with an EGFR specific VHH, 9G8, the ligand for which (EGFR) was not expressed on HEK Blue IL18 (data not shown). FIG. 25A and FIG. 25B are nonlinear x-y plots of IL18 biological activity versus the concentration of each test article using HEK Blue IL18 (FIG. 25A) and PD-1 decorated HEK Blue IL18 (FIG. 25B). As previously observed for pro-IL18AS and pro-IL18AV, serine substitutions results in greater attenuation than valine substitutions. As such, when HEK Blue IL18 (FIG. 25A; trans activity only), was treated with (a) the valine mutants FUSE783 (open reverse triangle) or FUSE775 (closed triangle), we observed about a 1000-fold attenuation relative to recombinant human IL18 and (b) the serine mutants FUSE782 (open triangle) and FUSE827 (closed reverse triangle), we observed about greater than a 100.000-fold attenuation relative to recombinant human IL18.

With regards to cis activity (FIG. 25B using PD-1 decorated HEK Blue IL18), we observed increased biological activity from the PD-1 targeted versions of pro-IL18AS (FUSE782; about 100-fold relative to non targeted FUSE827) and pro-IL18AV (FUSE783; approximately 30-fold relative to non targeted FUSE775).

FUSE691 (nivolumab) was used as the negative control antibody in both FIG. 25A and FIG. 25B. No biological activity was observed from this test article. No appreciable difference was observed in the biological activities of recombinant human IL18 or the non targeted test articles (FUSE775 and FUSE827), allowing for comparison across FIG. 25A and FIG. 25B. In this context, the PD-1 targeted versions of pro-IL18AS (FUSE782) and pro-IL18AV (FUSE783) were about 100 and 30-fold more active when exposed to PD-1 decorated HEK Blue IL-18 than non-decorated HEK Blue L18.

Summary tables of the potency of each pro-IL18 variant are shown below each graph.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used to describe and claim the invention, the present invention, or embodiments thereof, may alternatively be described using other terms such as “consisting of” or “consisting essentially of”.

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application. “Optional” or “optionally” means that the subsequently described circumstance may or may not occur, so that the description includes instances where the circumstance occurs and instances where it does not. Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. (canceled)

2. (canceled)

3. (canceled)

4. A method of treating cancer in a subject in need thereof, comprising:
administering a fusion protein to a subject in need thereof,
wherein the fusion protein comprises a first polypeptide or protein capable of translocating into an endoplasmic reticulum (ER) or a fragment thereof; and an interleukin 18 (IL-18), a fragment of IL-18, an IL-18 variant, or a fragment of the IL-18 variant,
wherein the IL-18, the fragment of IL-18, the IL-18 variant, or the fragment of the IL-18 variant is on the C-terminus end of the fusion protein relative to the first polypeptide or protein capable of translocating into the ER.

5. (canceled)

6. (canceled)

7. (canceled)

8. The method of claim 4, wherein the fusion protein further comprises a targeting polypeptide.

9. The method of claim 8, wherein the targeting polypeptide targets a protein on a cell surface.

10. (canceled)

11. The method of claim 4, wherein the fusion protein binds to a cell having an IL-18 RC or capable of expressing the IL-18 RC upon activation of the cell, and activates the IL-18 RC signal.

12. The method of claim 8, wherein the targeting polypeptide comprises a tumor associated antigen binding domain.

13. The method of claim 4, wherein the fusion protein further comprises an antibody or antibody fragment.

14. The method of claim 13, wherein antibody fragment is an Fc fragment.

15. The method of claim 4, wherein the fusion protein further comprises a masking domain.

16. The method of claim 4, wherein a mature IL-18 or mature IL-18 variant is released from a masking domain by a protease.

17. (canceled)

18. (canceled)

19. (canceled)

20. (canceled)

21. (canceled)

22. The method of claim 4, wherein the fusion protein further comprises half-life extending molecule.

23. (canceled)

24. (canceled)

25. (canceled)

26. (canceled)

27. (canceled)

28. (canceled)

29. (canceled)

30. (canceled)

31. The method of claim 4, wherein the fusion protein, the IL-18, the fragment of IL-18, the IL-18 variant, or the fragment of the IL-18 variant is fused to a terminus of a knob of a knob-into-hole heterodimeric IgG1 protein.

32. The method of claim 31, wherein the fusion protein, the IL-18, a fragment of IL-18, an IL-18 variant, or a fragment of the IL-18 variant further comprises a propeptide (PP) or a PP variant.

33. The method of any claim 32, wherein the IL-18 propeptide variant comprises a polypeptide having AAEPVEDNX₁INFVAMKFIDNTLYFIAEDDEN (SEQ ID NO:238), wherein X₁ is not cysteine.

34. (canceled)

35. (canceled)

36. (canceled)

37. (canceled)

38. (canceled)

39. (canceled)

40. (canceled)

41. The method of claim 4, wherein the fusion protein further comprises one or more cleavage sites and the fusion protein is cleaved at the one or more cleavage sites by one or more proteases.

42. The method of claim 41, wherein the one or more cleavage sites is
between the IL-18, a fragment of IL-18, an IL-18 variant, or a fragment of the IL-18 variant and the first protein capable of translocating into an endoplasmic reticulum (ER) or fragment thereof, or
within the PP, between PP or the PP variant and the IL-18, a fragment of IL-18, an IL-18 variant, or a fragment of the IL-18 variant, or
within the PP, between the PP or the PP variant and the first protein capable of translocating into an endoplasmic reticulum (ER) or fragment thereof, or
within the IL-18, a fragment of IL-18, an IL-18 variant, or a fragment of the IL-18 variant, or within the PP, or
a combination thereof.

43. The method of claim 4, wherein the fusion protein further comprises a second protein capable of translocating into the ER or a fragment thereof, wherein the second protein capable of translocating into the ER is on the C-terminus end relative to the interleukin 18 (IL-18), the fragment of the IL-18, the IL-18 variant, or the fragment of the IL-18 variant.

44. (canceled)

45. (canceled)

46. The method of claim 4, wherein the fusion protein comprises the second protein capable of translocating into the ER or a fragment thereof fused to the C-terminus of the interleukin 18 (IL-18), the fragment of the IL-18, the IL-18 variant, or the fragment of the IL-18 variant.

47. The method of claim 4, wherein the fusion protein comprises the IL-18 variant having diminished binding to IL-18 binding protein (IL-18BP), as compared to a wild type (wt) IL-18.

48. (canceled)

49. (canceled)

50. The method of claim 4, wherein the first protein capable of translocating into the ER or a fragment thereof of the fusion protein is a globular protein, immunoglobular protein, or a fragment thereof, or is a short polypeptide or protein engineered with a signal peptide for translocating into the ER, optionally the short polypeptide or protein being about 2 kDa or no greater than 250 kDa, or optionally, the short polypeptide being 1, 2, 3, or 4 amino acids, or being a flexible linker of amino acid chain of 5, 6, 7, 8, 9, 10, 11, 12, 13, 14, 15, 16, 17, 18, 19, 20, 25, 30, or 35 amino acids, optionally the short polypeptide being selected from the group consisting of T, PT, MPT, S, GS, GGS, GGGS (SEQ ID NO:235), (GGGGX_{λ} (SEQ ID NO:236))ₙ wherein X_{λ} is Q, A, E or S and n=1-5, (X_{λ}GGGG (SEQ ID NO:317))n wherein X_{λ} is Q, A, E or S and n=1-5, (GX_{λ}GGG (SEQ ID NO:318))ₙ wherein X_{λ} is Q, A, E or S and n=1-5, (GGX_{λ}GG (SEQ ID NO:319))ₙ wherein X_{λ} is Q, A, E or S and n=1-5, (GGGX_{λ}G (SEQ ID NO:320))ₙ wherein X_{λ} is Q, A, E or S and n=1-5, (X_{λ}GGG)ₙ wherein X_{λ} is Q, A, E or S and n=1-5, (GX_{λ}GG)ₙ wherein X_{λ} is Q, A, E or S and n=1-5, (GGX_{λ}G)ₙ wherein X_{λ} is Q, A, E or S and n=1-5, (GGGX_{λ})ₙ wherein X_{λ} is Q, A, E or S and n=1-5, (X_{λ}GG)ₙ wherein X_{λ} is Q, A, E or S and n=1-5, (GX_{λ}G)ₙ wherein X_{λ} is Q, A, E or S and n=1-5, (GGX_{λ})ₙ wherein X_{λ} is Q, A, E or S and n=1-5, (X G)ₙ wherein X is Q, A, E or S and n=1-5, and (GX_{λ})ₙ wherein X_{λ} is Q, A, E or S and n=1-5.

51. (canceled)

52. (canceled)

53. (canceled)

54. (canceled)

55. The method of claim 46, wherein the second protein capable of translocating into the ER or a fragment thereof of the fusion protein is selected from the group consisting of a fragment crystallizable (Fc) region, human serum albumin (HSA), beta2microglobulin, transferrin, fragment antigen-binding region (Fab region), VHH antibody, single-chain variable fragment (scFv), anticalin, designed ankyrin repeat protein (DARPin), a binding domain thereof, and a fragment thereof.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The method of claim 4, wherein the fusion protein further comprises a tumor associated antigen binding domain.

74. (canceled)

75. (canceled)

76. The method of claim 4, wherein the fusion protein comprises one or more sequences as set forth in any one in Tables 1A, 1B, and 4.

77. A fusion protein comprising a mature IL-18 variant selected from Table 1B; and a short polypeptide.

78. The fusion protein of claim 78, wherein the IL-18 variant is from FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, or FUSE-1177.

79. An IL-18 variant selected from Table 1B corresponding to FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177.

80. A fusion protein comprising an IL-18 variant of claim 79, wherein the fusion protein is selected from the group consisting of FUSE-557, FUSE-626, FUSE-669, FUSE-670, FUSE-675, FUSE-676, FUSE-682, FUSE-683, FUSE-684, FUSE 685, FUSE 832, FUSE 882, FUSE 895, FUSE 952, FUSE-1015, FUSE-1016, FUSE-1017, FUSE-1041, FUSE-1042, FUSE-1044, FUSE-1109, FUSE-1110, FUSE-1111, FUSE-1112, FUSE-1113, FUSE 1116, FUSE-1176, and FUSE-1177.
